(19) United States
(12) Patent Application Publication
(10) Pub. No.: US 2024/0174722 A1
(43) Pub. Date: May 30, 2024

(54) SYNTHETIC SIGNAL PEPTIDES FOR DIRECTING SECRETION OF HETEROLOGOUS PROTEINS IN YEAST

(71) Applicant: TENZA, INC., Bostion, MA (US)

(72) Inventors: Anik Debnath, Somerville, MA (US); Ameet Shetty, Natick, MA (US)

(21) Appl. No.: 18/281,117

(22) PCT Filed: Mar. 11, 2022
(86) PCT No.: PCT/US2022/019962

§ 371 Date: Sep. 8, 2023

Related U.S. Application Data
(60) Provisional application No. 63/159,843, filed on Mar. 11, 2021.
(60) Provisional application No. 63/221,041, filed on Jul. 13, 2021.

(51) Int. Cl. C07K 14/395; A61K 36/064; A61K 38/00; C12N 1/18; C12R 1/685; C12R 1/84; C12R 1/87; C12R 1/885

(52) U.S. Cl. CPC C07K 14/395; A61K 36/064; C12N 1/185; A61K 38/00; C07K 2319/02; C12R 2001/685; C12R 2001/84; C12R 2001/87; C12R 2001/885

(57) ABSTRACT

Provided herein are signal peptides that direct secretion of expressed payload proteins in yeast. Methods of using the signal peptides for therapeutic and non-therapeutic utilities are also provided. Compositions comprising yeast comprising the signal peptides and methods of using said yeast comprising the signal peptides for therapeutic and non-therapeutic utilities are also provided. Methods to design and generate the disclosed signal peptides are also provided.

### SEQUENCE LISTING

The instant application contains a Sequence Listing which has been submitted electronically in ASCII format and is hereby incorporated by reference in its entirety. Said ASCII copy, created on Apr. 15, 2022 is named 257723_000402_SL.txt and is 77,218 bytes in size.

### FIELD

The present disclosure relates generally to signal peptides and more particularly to synthetic signal peptides that increase secretion of a recombinant protein.

### BACKGROUND

Yeasts are routinely used as hosts to produce proteins for research, therapeutic and industrial purposes. Once produced, a protein is usually translocated into the endoplasmic reticulum (ER), then transported to the Golgi, then secreted into the extracellular space. Movement along this secretory pathway is facilitated by a signal peptide which usually comprises about 16-30 amino acids and is fused to the N-terminus of the protein. However, despite considerable efforts to genetically optimize the synthesis of recombinant proteins by yeast, optimization of the chaperone pathways used by a synthesized protein to reach the extracellular space are comparatively fewer and have rarely been successful. The generation capacity of a yeast, therefore, remains too small to be viable for industrial-type applications and is thus limited to smaller scale processes.

The most common signal peptide used currently is the α-mating factor pro-protein signal peptide α-MF, from Saccharomyces cerevisiae. Its performance varies greatly depending on the payload protein. Only direct experimental assessment, with consequent expenditure of time and resources, provides assessment of its performance with any particular payload protein. Therefore, α-MF is usually implemented as is, not only in S. cerevisiae, but also in orthologous yeast strains, therefore compounding the unpredictability and challenge to effectively produce a recombinant protein in yeast. Some efforts to optimize secretion have been made but most, if not all have relied on either empirical design or directed evolution which are laborious and small scale method and require a native signal peptide as a starting template. A need therefore exists for engineering a system that not only increases the secretion of a recombinant protein produced in yeast, but has application across numerous yeast species.

### SUMMARY

In some embodiments, a pre-protein signal peptide is provided. In some embodiments, the pre-protein signal peptide comprises an amino acid sequence selected from the group consisting of Formula I, Formula II, Formula III, Formula IV, Formula V, Formula IX, and Formula XIII.

In certain embodiments, Formula I is represented by: A₁-(A₂)_{w}-A₃-(A₄)ₓ-(A₅)_{y}-A₆-A₇-A₈-A₉-A₁₀-(A₁₁)_{z} (Formula I) as described herein.

In certain embodiments, Formula II is represented by: B₁-(B₂)ᵤ-(B₃)ᵥ-(B₄)_{w}-(B₅)ₓ-(B₆)_{y}-B₇-B₈-B₉-B₁₀-(B₁₁)_{z} (Formula II) as described herein.

In certain embodiments, Formula III is represented by: C₁-(C₂)ᵣ-(C₃)ₜ-(C₄)ᵤ-[(C₅)ᵥ-(C₆)_{w}]ₓ-(C₇)_{y}-(C₈)_{z}-C₉-C₁₀-C₁₁-[C₁₂-C₁₃]ₐ (Formula III) as described herein.

In certain embodiments, Formula IV is represented by: D₁-(D₂)_{q}-(D₃)ᵣ-(D₄)ₜ-(D₅)ᵤ-[(D₆)ᵥ-(D₇)ₓ-(D₈)_{w}-(D₉)_{y}]_{z}-D₁₀-D₁₁-D₁₂-[D₁₃-D₁₄]ₐ (Formula IV) as described herein.

In certain embodiments, Formula V is represented by: E₁-[(E₂)ᵢ-(E₃)ⱼ-(E₄)_{q}]ᵣ-(E₅)ₜ-(E₆)ᵤ-(E₇)ᵥ-[(E₈)_{w}-(E₉)ₓ]_{y}-(E₁₀)_{z}-E₁₁-E₁₂-E₁₃-[E₁₄-E₁₅]ₐ (Formula V) as described herein.

In certain embodiments, Formula IX is represented by: F₁-(F₂)ᵥ-(F₃)_{w}-[(F₄)ₓ-(F₅)_{y}]_{z}-F₆-F₇-F₈-[F₉-F₁₀]ₐ (Formula IX) as described herein.

In certain embodiments, Formula XIII is represented by: L₁-(L₂)ₓ-[(L₃)ₐ-(L₄)ₐ]_{y}-[(L₅)ₐ-(L₆)ₐ-(L₇)ₐ]_{z}-(L₈)ₐ-(L₉)ₐ-(L₁₀)ₐ-(L₁₁)ₐ-(L₁₂)ₐ (Formula XIII) as described herein.

In some embodiments, a pre-protein signal peptide is provided. In some embodiments, the pre-protein signal peptide comprises an amino acid sequence having at least 90%, 91%, 92%, 93%, 94%, 95%, 96%, 97%, 98%, 99% or 100% identity to SEQ ID NO. 1, 2, 3, 4, 5, 6, 7, 8, 9, 10, 11, 12, 13, 14, 15, 16, 28, 31, 32, 33, 55, 70, 71, 72, or 73.

In some embodiments, a pro-protein signal peptide is provided. In some embodiments, the pro-protein signal peptide comprises an amino acid sequence selected from the group consisting of Formula VI, Formula VII, Formula VIII, Formula X, Formula XI, Formula XIV, and Formula XV.

In certain embodiments, Formula VI is represented by: G₁-G₂-G₃-G₄-G₅-G₆-G₇-G₈-G₉-G₁₀-G₁₁-G₁₂-G₁₃-G₁₄-G₁₅-G₁₆-G₁₇-G₁₈-G₁₉-G₂₀-G₂₁-G₂₂-G₂₃-G₂₄-G₂₅ (Formula VI) as described herein.

In certain embodiments, Formula VII is represented by: (H₁)ₘ-(H₂)ₘ-(H₃)ₘ-(H₄)ₘ-(H₅)ₘ-(H₆)ₘ-(H₇)ₘ-(H₈)ₘ-(H₉)ₘ-(H₁₀)ₘ-(H₁₁)ₘ-(H₁₂)ₘ-(H₁₃)ₘ-(H₁₄)ₘ- (H₁₅)ₘ-(H₁₆)ₘ-(H₁₇)ₘ-(H₁₈)ₘ-(H₁₉)ₘ-(H₂₀)ₘ-(H₂₁)ₘ-(H₂₂)ₘ-(H₂₃)ₘ-(H₂₄)ₘ-(H₂₅)ₘ-(H₂₆)ₘ-(H₂₇)ₘ-(H₂₈)ₘ-(H₂₉)ₘ-(H₃₀)ₘ-(H₃₁)ₘ-(H₃₂)ₘ-(H₃₃)ₘ-(H₃₄)ₘ-(H₃₅)ₘ-(H₃₆)ₘ-H₃₇-H₃₈-H₃₉-H₄₀ (Formula VII) as described herein.

In certain embodiments, Formula VIII is represented by: (I₁)ₘ-(I₂)ₘ-(I₃)ₘ-(I₄)ₘ-(I₅)ₘ-(I₆)ₘ-(I₇)ₓ-(I₈)ₘ-(I₉)ₘ-(I₁₀)ₘ-(I₁₁)ₓ-(I₁₂)ₘ-(I₁₃)ₓ-(I₁₄)ₓ- (I₁₅)ₘ-(I₁₆)ₓ-(I₁₇)ₘ-I₁₈-I₁₉-I₂₀-I₂₁-I₂₂-I₂₃ (Formula VIII) as described herein.

In certain embodiments, Formula X is represented by: (J₁)_{z}-(J₂)_{z}-(J₃)_{z}-(J₄)_{z}-(J₅)_{z}-(J₆)_{z}-(J₇)_{z}-(J₈)_{z}-(J₉)_{z}-(J₁₀)_{z}-(J₁₁)_{z}-(J₁₂)_{z}-(J₁₃)_{z}-(J₁₄)_{z}-(J₁₅)_{z}- (J₁₆)_{z}-(J₁₇)_{z}-(J₁₈)_{z}-(J₁₉)_{z}-(J₂₀)_{z}-(J₂₁)_{z}-J₂₂-J₂₃-J₂₄-J₂₅ (Formula X) as described herein.

In certain embodiments, Formula XI is represented by: (K₁)_{b}-(K₂)_{b}-(K₃)_{b}-(K₄)_{b}-(K₅)_{b}-(K₆)_{b}-(K₇)_{b}-(K₈)_{b}-(K₉)_{b}-(K₁₀)_{b}-(K₁₁)_{b}-(K₁₂)_{b}-(K₁₃)_{b}-(K₁₄)_{b}- (K₁₅)_{b}-(K₁₆)_{b}-(K₁₇)_{b}-(K₁₈)_{b}-(K₁₉)_{b}-(K₂₀)_{b}-(K₂₁)_{b}-(K₂₂)_{b}-(K₂₃)_{b}-(K₂₄)_{b}-(K₂₅)_{b}-(K₂₆)_{b}-(K₂₇)_{b}-(K₂₈)_{b}-(K₂₉)_{b}-(K₃₀)_{b}-(K₃₁)_{b}-(K₃₂)_{b}-(K₃₃)_{b}-(K₃₄)_{b}-(K₃₅)_{b}-(K₃₆)_{b}-(K₃₇)_{b}-(K₃₈)_{b}-(K₃₉)_{b}-(K₄₀)_{b}-(K₄₁)_{b}-(K₄₂)_{b}-(K₄₃)_{b}-(K₄₄)_{b}-(K₄₅)_{b}-(K₄₆)_{b}-(K₄₇)_{b}-(K₄₈)_{b}-(K₄₉)_{b}-(K₅₀)_{b}- (K₅₁)_{b}-(K₅₂)_{b}-(K₅₃)_{b}-(K₅₄)_{b}-(K₅₅)_{b}-(K₅₆)_{b}-(K₅₇)_{b}-(K₅₈)_{b}-(K₅₉)_{b}-(K₆₀)_{b}-(K₆₁)_{b}-(K₆₂)_{b}-(K₆₃)_{b}-(K₆₄)_{b}-(K₆₅)_{b}-(K₆₆)_{b}-(K₆₇)_{b}-(K₆₈)_{b}-(K₆₉)_{b}-(K₇₀)_{b}-(K₇₁)_{b}-(K₇₂)_{b}-(K₇₃)_{b}-(K₇₄)_{b}-(K₇₅)_{b}-(K₇₆)_{b}-(K₇₇)_{b}-(K₇₈)_{b}-(K₇₉)_{b}-(K₈₀)_{b}-(K₈₁)_{b}-(K₈₂)_{b}-(K₈₃)_{b}-(K₈₄)_{b}-(K₈₅)_{b}-(K₈₆)_{b}-(K₈₇)_{b}-(K₈₈)_{b}-K₈₉-K₈₉-K₈₉-K₈₉-K₈₉ (Formula XI) as described herein.

In certain embodiments, Formula XIV is represented by: (M₁)_{b}-(M₂)_{b}-(M₃)_{b}-(M₄)_{b}-(M₅)_{b}-(M₆)_{b}-(M₇)_{b}-(M₈)_{b}-(M₉)_{b}-(M₁₀)_{b}-(M₁₁)_{b}-(M₁₂)_{b}-(M₁₃)_{b}-(M₁₄)_{b}- (M₁₅)_{b}-(M₁₆)_{b}-(M₁₇)_{b}-(M₁₈)_{b}-(M₁₉)_{b}-(M₂₀)_{b}-(M₂₁)_{b}-(M₂₂)_{b}-(M₂₃)_{b}-(M₂₄)_{b}-(M₂₅)_{b}-(M₂₆)_{b}-(M₂₆)_{b}-(M₂₇)_{b}-(M₂₈)_{b}-(M₂₉)_{b}-(M₃₀)_{b}-(M₃₁)_{b}-(M₃₂)_{b}-(M₃₃)_{b}-(M₃₄)_{b}-(M₃₅)_{b}-(M₃₆)_{b}-(M₃₇)_{b}-(M₃₈)_{b}-(M₃₉)_{b}-(M₄₀)_{b}-(M₄₁)_{b}-(M₄₂)_{b}-(M₄₃)_{b}-(M₄₄)_{b}-(M₄₅)_{b}-(M₄₆)_{b}-(M₄₇)_{b}-(M₄₈)_{b}-(M₄₉)_{b}-(M₅₀)_{b}-(M₅₁)_{b}-(M₅₂)_{b}-(M₅₃)_{b}-(M₅₄)_{b}-(M₅₅)_{b}-(M₅₆)_{b}-(M₅₇)_{b}-(M₅₈)_{b}-(M₅₉)_{b}-(M₆₀)_{b}-(M₆₁)_{b}-(M₆₂)_{b}-(M₆₃)_{b}-(M₆₄)_{b} (M₆₅)_{b}-(M₆₆)_{b}-(M₆₇)_{c}-(M₆₈)_{c}-(M₆₉)_{c}-(M₇₀)_{c} (Formula XIV) as described herein

In certain embodiments, Formula XV is represented by: (N₁)_{b}-(N₂)_{b}-(N₃)_{b}-(N₄)_{b}-(N₅)_{b}-(N₆)_{b}-(N₇)_{b}-(N₈)_{b}-(N₉)_{b}-(N₁₀)_{b}-(N₁₁)_{b}-(N₁₂)_{b}-(N₁₃)_{b}-(N₁₄)_{b}- (N₁₅)_{b}-(N₁₆)_{b}-(N₁₇)_{b}-(N₁₈)_{b}-(N₁₉)_{b}-(N₂₀)_{b}-(N₂₁)_{b}-(N₂₂)_{b}-(N₂₃)_{b}-(N₂₄)_{b}-(N₂₅)_{b}-(N₂₆)_{b}-(N₂₇)_{b}-(N₂₈)_{b}-(N₂₉)_{b}-(N₃₀)_{b}-(N₃₁)_{b}-(N₃₂)_{b}-(N₃₃)_{b}-(N₃₄)_{b}-(N₃₅)_{b}-(N₃₆)_{b}-(N₃₇)_{b}-(N₃₈)_{b}-(N₃₉)_{b}-(N₄₀)_{b}-(N₄₁)_{b}-(N₄₂)_{b}-(N₄₃)_{b}-(N₄₄)_{b}-(N₄₅)_{b}-(N₄₆)_{b}-(N₄₇)_{b}-(N₄₈)_{b}-(N₄₉)_{b}-(N₅₀)_{b}-(N₅₁)_{b}-(N₅₂)_{b}-(N₅₃)_{b}-(N₅₄)_{b}-(N₅₅)_{b}-(N₅₆)_{b}-(N₅₇)_{b}-(N₅₈)_{b}-(N₅₉)_{b}-(N₆₀)_{b}-(N₆₁)_{b}-(N₆₂)_{b}-(N₆₃)_{b}-(N₆₄)_{b}-(N₆₅)_{b}-(N₆₆)_{b}-(N₆₇)_{c}-(N₆₈)_{c}-(N₆₉)_{c}-(N₇₀)_{c}-(N₇₁)_{c} (Formula XV) as described herein.

In some embodiments, a pro-protein signal peptide is provided. In some embodiments, the pro-protein signal peptide comprises an amino acid sequence having at least 90%, 91%, 92%, 93%, 94%, 95%, 96%, 97%, 98%, 99% or 100% identity to an amino acid sequence selected from the group consisting of SEQ ID NO. 17, 18, 19, 20, 21, 22, 23, 24, 25, 27, 29, 34, 35, 36, 37, 38, 56, 57, 58, 74, or 75.

In some embodiments, a pre-protein plus a pro-protein signal peptide is provided. In some embodiments, the pre-protein plus a pro-protein signal peptide comprises an amino acid sequence having at least 90%, 91%, 92%, 93%, 94%, 95%, 96%, 97%, 98%, 99% or 100% identity to an amino acid sequence of SEQ ID NO: 30.

In some embodiments, a polypeptide is provided. In some embodiments, the recombinant polypeptide comprises a formula of (X₁)ₙ-(Y₁)ₘ-Z₁, wherein X₁ is a pre-protein signal peptide, Y₁ is a pro-protein signal peptide, and Z₁ is a payload protein, wherein n is 0 or 1 and m is 0 or 1, and wherein n and m cannot concurrently be 0.

In some embodiments, a yeast is provided. In some embodiments, the yeast comprises a heterologous nucleic acid molecule encoding a polypeptide having a formula of (X1)ₙ-(Y1)m-Z1, wherein X1 is a pre-protein signal peptide as provided for herein, Y1 is a pro-protein signal peptide as provided for herein, and Z1 is a payload protein, wherein n is 0 or 1 and m is 0 or 1, and wherein n and m cannot concurrently be 0.

In some embodiments, a method for producing a payload protein is provided. In some embodiments, the method comprises transfecting a yeast with a nucleic acid encoding a recombinant polypeptide as provided for herein, producing an engineered yeast, culturing the engineered yeast in an environment effective to grow the engineered yeast, and inducing secretion of the payload protein by the engineered yeast.

In some embodiments, a method for treating a disease or condition in a subject in need thereof is provided. In some embodiments, the method comprises administering to the subject a therapeutically effective amount of a yeast as provided for herein.

### DESCRIPTION OF THE DRAWINGS

The foregoing and other features of the disclosure will become more apparent from the following detailed description of several embodiments, which proceeds with reference to the accompanying figures.

FIG. 1 provides four recombinant polypeptide constructs representing combinations of synthetic pre-protein signal (sPre), synthetic pro-protein signal (sPro), and native pre-protein signal (nPre) peptides that may be utilized according to methods disclosed herein to increase secretion of a payload protein.

FIG. 2 provides western blots that depict the amount of maltose binding protein (MBP) in cell-free supernatant that were secreted by wild type and engineered K. lactis yeast.

FIG. 3A graphically depicts accumulation of MBP by engineered K. lactis yeast (expressing synthetic signal peptide synKlac-v1) versus wild-type K. lactis yeast over time.

FIG. 3B graphically depicts accumulation of MBP by wild type K. lactis yeast versus engineered K. lactis yeast (expressing synthetic signal peptide synKlac-v1) as a function of yeast growth (optical density).

FIG. 4 is a graph of MBP RNA expression in wild type K. lactis yeast versus engineered K. lactis yeast (expressing synthetic signal peptide synKlac-v1).

FIG. 5 is a graph of normalized TNF-α levels produced by wild type K. lactis yeast versus engineered K. lactis yeast (expressing synthetic signal peptide synKlac-v1).

FIG. 6 is a graph of normalized phytase levels generated by wild type P. pastoris (expressing native signal peptide (PHO1, α-MF) versus engineered P. pastoris yeast (expressing synthetic signal peptide synPichia-v1 or synPichia-v4).

FIG. 7 reports normalized insulin production by wild type S. cerevisiae yeast versus engineered S. cerevisiae yeast (expressing synthetic signal peptide synScer-v5). Insulin was quantified using ELISA and data were normalized to insulin mRNA levels for each variant tested. FIG. 7A reports the comparison between yeast utilizing the synScer-v5 signal peptide and yeast utilizing the α-MF signal peptide. FIG. 7B reports the comparison between yeast utilizing the synScer-v5 signal peptide and yeast expressing optYAP.

FIG. 8 reports normalized enzyme activity of purified invertase extracts generated by wild type S. boulardii yeast versus enzyme activity of purified invertase extracts generated by engineered S. boulardii yeast (expressing synthetic signal peptide synScer-v1). FIG. 8A reports invertase activity from invertase purified from the culture media. FIG. 8B reports invertase activity from invertase purified from periplasmic extracts.

FIG. 9 reports the activity of invertase generated by engineered S. boulardii yeast compared to the activity of commercially-available invertase at different pH levels. FIG. 9A reports the data from engineered S. boulardii. FIG. 9B reports the data from commercially available invertase.

FIG. 10 graphically depicts the change in glucose levels as an indirect measure of invertase activity over time as produced in wild type versus S. boulardii engineered to express invertase with the synthetic signal peptide synScer-v1.

FIG. 11 graphically depicts the amount of yeast in various GI tissues of mice orally administered engineered S. boulardii yeast.

FIG. 12 graphically depicts the activity of invertase generated by wild type S. boulardii versus enzyme activity of invertase generated by engineered S. boulardii yeast (expressing synthetic signal peptide synScer-v1).

FIG. 13 graphically depicts normalized IGF-1 production by wild type S. boulardii versus engineered S. boulardii yeast (expressing synthetic signal peptide synScer-v1, synScer-v3, or synScer-v5).

FIG. 14 graphically depicts normalized lysozyme production by wild type S. boulardii versus engineered S. boulardii yeast (expressing synthetic signal peptide synScer-v4 or synScer-v5).

FIG. 15 pictorially depicts survival of S. boulardii engineered to express payload protein (mCherry) deployment through the upper GI tract of mice over time.

FIG. 16 graphically depicts sucrase activity per CFU in lyophilized S. boulardii yeast engineered to express sucrase fused to synthetic signal peptide synScer-v1.

FIG. 17 graphically depicts the activity of sucrase expressed by S. boulardii yeast engineered to express sucrase fused to synthetic signal peptide synScer-v1 as a function of pH.

FIG. 18 graphically depicts the loss of sucrase activity in the presence of glucose of S. boulardii yeast engineered to express sucrase fused to synthetic signal peptide synScer-v1 in compared to sucrase expressed in wild type S. boulardii.

FIG. 19 graphically depicts the persistence of by S. boulardii yeast engineered to express sucrase fused to synthetic signal peptide synScer-v1 in the GI tissue over time.

FIG. 20 graphically depicts glucose excursion time curves of sucrose-challenged mice are administered boulardii yeast engineered to express sucrase fused to synthetic signal peptide synScer-v1.

FIG. 21 is AUC data from FIG. 20, represented in bar graph format.

FIG. 22 provides various recombinant polypeptide constructs representing various combinations of synthetic and native pre- and pro-protein signal peptides that may be utilized according to methods disclosed herein to improve secretion efficiency of invertase protein.

FIG. 23 reports a comparison between normalized invertase production by S. boulardii modified to express a recombinant polypeptide comprising of a native or S. cerevisiae signal (SBsyn-Scerv1) versus S. boulardii modified to express a recombinant polypeptide comprising various synthetic signal peptides from S. boulardii (SBsyn-Sbouv2, SBsyn-Sbouv3, SBsyn-Sbouv4).

FIG. 24 provides various recombinant polypeptide constructs representing various combinations of synthetic and native pre- and pro-protein signal peptides that may be utilized according to methods disclosed herein to improve secretion efficiency of lysozyme protein.

FIG. 25 reports a comparison between normalized lysozyme production by S. boulardii modified to express a recombinant polypeptide comprising of a chicken lysozyme signal sequence versus S. boulardii modified to express a recombinant polypeptide comprising various synthetic signal peptides from S. boulardii (SBsyn-Sbouv)

FIG. 26 provides the recombinant polypeptide construct representing a combination of synthetic pre- and pro-protein signal peptides that may be utilized according to methods disclosed herein to improve secretion efficiency of beta-galactosidase protein.

FIG. 27 graphically depicts normalized beta-galactosidase production by S. boulardii modified to express a recombinant polypeptide comprising a synthetic signal peptide from S. boulardii (SBsyn-Sbouv2)

FIG. 28 provides various recombinant polypeptide constructs representing various combinations of synthetic and native pre- and pro-protein signal peptides that may be utilized according to methods disclosed herein to improve secretion efficiency of anti-TNFα protein.

FIG. 29 graphically depicts normalized anti TNFα activity production by S. boulardii modified to express a recombinant polypeptide comprising a synthetic signal peptide from S. boulardii (SBsyn-Sbouv1 and SBsyn-Sbouv2).

FIG. 30 graphically depicts the use of S. boulardii cells to secrete anti-TNFα antibody fragments. FIG. 30A reports the secretion of monovalent anti-TNFα antibody fragments. FIG. 30B reports the secretion of bivalent anti-TNFα antibody fragments.

FIG. 31 compares the secretion of invertase by S. boulardii cells that transiently express a Sbouv2-invertase polypeptide and S. boulardii cells that were engineered for stable and reliable expression of invertase by integrating copies of constructs containing the Sbouv2 synthetic signal peptide fused to the invertase into the S. boulardii genome.

FIG. 32 provides various recombinant polypeptide constructs representing various combinations of synthetic and native pre- and pro-protein signal peptides that may be utilized according to methods disclosed herein to improve secretion efficiency of the LCRF protein.

FIG. 33 graphically depicts normalized LCRF production by S. boulardii modified to express a recombinant fusion protein comprising a synthetic signal peptide from S. boulardii.

### DETAILED DESCRIPTION

The present disclosure presents a solution to the aforementioned challenges by providing new, synthetic signal peptides that direct secretion of expressed proteins or peptides in yeast. The disclosed signal peptides overcome performance variability challenges posed by previously characterized and native signal peptides and may be used to generate and facilitate secretion of any protein or peptide from a yeast.

The disclosed synthetic pre-protein (sPre) signal peptides and synthetic pro-protein (sPro) signal peptides increase secretion of any recombinant protein in yeast. Increased secretion can be advantageously achieved with a synthetic pre-protein signal peptide alone, with a synthetic pro-protein signal peptide alone, or with both. In any embodiment, a synthetic pre-protein signal peptide may be used in combination with a native pro-protein (nPro) signal peptide or sPro signal peptide. Likewise, in any embodiment, a synthetic pro-protein signal peptide may be used in combination with a native pre-protein (nPre) signal peptide or an sPre signal peptide. The use of synthetic pro-protein signal peptide together with a synthetic pre-protein signal peptide may further improve secretion of a payload protein, for example, through facilitating Golgi-trafficking. Advantageously, the signal peptides disclosed herein have been generated and optimized to promote secretion of any payload protein from a yeast. Use of the disclosed synthetic pre-protein signal peptides and synthetic pro-protein signal peptides may be used to achieve increased secretion of any desired payload to any yeast-compatible environment, such as in therapeutics, agriculture, or food products.

Before the present compositions and methods are described, it is to be understood that the scope of the invention is not limited to the particular processes, compositions, or methodologies described herein, as these may vary. It is also to be understood that the terminology used in the description is for the purpose of describing the particular versions or embodiments only, and is not intended to limit the scope of the present invention. Unless defined otherwise, all technical and scientific terms used herein have the same meanings as commonly understood by one of ordinary skill in the art. Although any methods and materials similar or equivalent to those described herein can be used in the practice or testing of embodiments of the methods and systems disclosed herein, the preferred methods, devices, and materials are now described.

### Definitions

The following explanations of terms and methods are provided to better describe the present disclosure and to guide those of ordinary skill in the art in the practice of the present disclosure.

As used herein, “comprising” means “including” and the singular forms “a” or “an” or “the” include plural references unless the context clearly dictates otherwise. For example, reference to “comprising a therapeutic agent” includes one or a plurality of such therapeutic agents. The term “or” refers to a single element of stated alternative elements, unless the context clearly indicates otherwise. For example, the phrase “A or B” refers to A alone or B alone. The phrase “A, B, or a combination thereof” refers to A alone, B alone, or a combination of A and B. Similarly, “one or more of A and B” refers to A, B, or a combination of both A and B. The phrase “A and B” refers to a combination of A and B. Furthermore, the various elements, features and steps discussed herein, as well as other known equivalents for each such element, feature or step, can be mixed and matched by one of ordinary skill in this art to perform methods in accordance with principles described herein. Among the various elements, features, and steps some will be specifically included and others specifically excluded in particular examples.

Unless otherwise defined, all technical and scientific terms used herein have the same meaning as commonly understood to one of ordinary skill in the art. Although methods and materials similar or equivalent to those described herein can be used in the practice or testing of the present disclosure, suitable methods and materials are described below. The materials, methods, and examples are illustrative only and not intended to be limiting. All references cited herein are incorporated by reference in their entirety.

In some examples, the numbers expressing quantities of ingredients, properties such as molecular weight, reaction conditions, and so forth, used to describe and claim certain embodiments are to be understood as being modified in some instances by the term “about” or “approximately.” For example, “about” or “approximately” can indicate +/−5% variation of the value it describes. Accordingly, in some embodiments, the numerical parameters set forth herein are approximations that can vary depending upon the desired properties for a particular embodiment. Notwithstanding that the numerical ranges and parameters setting forth the broad scope of some examples are approximations, the numerical values set forth in the specific examples are reported as precisely as practicable. The recitation of ranges of values herein is merely intended to serve as a shorthand method of referring individually to each separate value falling within the range.

To facilitate review of the various embodiments of this disclosure, the following explanations of specific terms are provided:

As used herein, “yeast” refers to a microscopic fungus consisting of cells that reproduce by budding and are capable of converting sugar into alcohol and carbon dioxide. The yeast, as disclosed herein may be genetically modified to induce expression of a heterologous payload protein. As used herein, “genetically modified” or any grammatical variation thereof, refers to a practice of introducing a nucleic acid or a nucleic acid molecule into a yeast cell that encodes and promotes the expression of a recombinant protein. The nucleic acid may be introduced transiently, or the nucleic acid may be incorporated into the genome of the yeast for stable expression. As used herein, the terms “nucleic acid” and “nucleic acid molecule” can be used interchangeably. The nucleic acid or nucleic acid molecule can be of any length. A nucleic acid may be DNA, mRNA, tRNA, or rRNA. A nucleic acid or nucleic acid molecule is composed of nucleotide monomers, each triplet of monomers (a codon) encoding for either a triplet of RNA nucleotide monomers (if the nucleic acid is DNA) or an amino acid (if the nucleic acid is RNA). DNA also comprises one or more promoter regions, which indicate where transcription of the DNA should start. mRNA also comprises a ribosome binding site, which indicates where translation of the mRNA should start as well as one or more stop codons, which indicates where mRNA translation should end. The introduction of a nucleic acid or nucleic acid molecule into a yeast cell can be accomplished by any method known in the art. Such methods are described in greater detail below.

In any embodiment or aspect disclosed herein, a nucleic acid encoding for a recombinant polypeptide, as disclosed herein, may be introduced into a yeast cell using any method known to those skilled in the art for such introduction. Such methods include transfection, transformation, transduction, infection (e.g., viral transduction), injection, microinjection, gene gun, nucleofection, nanoparticle bombardment, transformation, conjugation, by application of the nucleic acid in a gel, oil, or cream, by electroporation, using lipid-based transfection reagents, or by any other suitable transfection method. One of skill in the art will readily understand and adapt such methods using readily identifiable literature sources.

As used herein, the terms “transformation” and “transfection” are intended to refer to a variety of art-recognized techniques for introducing foreign nucleic acid into a host cell, including calcium phosphate or calcium chloride co-precipitation, DEAE-dextran-mediated transfection, lipofection (e.g., using commercially available reagents such as, for example, LIPOFECTIN® (Invitrogen Corp., San Diego, CA), LIPOFECTAMINE® (Invitrogen), FUGENE® (Roche Applied Science, Basel, Switzerland), JETPEI™ (Polyplus-transfection Inc., New York, NY), EFFECTENE® (Qiagen, Valencia, CA), DREAMFECT™ (OZ Biosciences, France) and the like), or electroporation (e.g., in vivo electroporation). Suitable methods for transforming or transfecting host cells can be found in Sambrook, et al. (Molecular Cloning: A Laboratory Manual. 2nd, ed., Cold Spring Harbor Laboratory, Cold Spring Harbor Laboratory Press, Cold Spring Harbor, N.Y., 1989), and other laboratory manuals.

Methods and materials of non-viral delivery of nucleic acids to cells further include biolistics, virosomes, liposomes, immunoliposomes, polycation or lipid-nucleic acid conjugates, naked DNA, artificial virions, and agent-enhanced uptake of DNA. Lipofection is described in U.S. Pat. Nos. 5,049,386, 4,946,787; and 4,897,355 and lipofection reagents are sold commercially (e.g., TRANSFECTAM™ and LIPOFECTIN™). Cationic and neutral lipids that are suitable for efficient receptor-recognition lipofection of polynucleotides include those disclosed in WO91/17424 and WO 91/16024.

The methods described herein comprise generating a recombinant polypeptide within a yeast host. As used herein, heterologous or recombinant describes a protein or nucleic acid that is not naturally found in or produced by the host yeast. As used herein, a “recombinant polypeptide” comprises a payload protein and a synthetic signal peptide fused directly or indirectly thereto. As used herein, “recombinant polypeptide” and “recombinant fusion protein” may be used interchangeably in the context of polypeptides comprising at least a first and second component (e.g. a synthetic signal peptide and a payload protein). As used herein, a signal peptide is any protein or peptide fused directly or indirectly to the N-terminus of a payload protein that facilitates the extracellular secretion of the payload protein after it is generated. A signal peptide may comprise one or more of a pre-protein signal peptide and pro-protein signal peptide.

While not wishing to be bound by theory, it is thought that the synthetic pre-protein signal peptides disclosed herein facilitate efficient translocation of the protein from a ribosome to the endoplasmic reticulum, and that the synthetic pro-protein signal peptides disclosed herein facilitate trafficking of the protein from the ER to the Golgi apparatus for eventual secretion. Pro-protein signal peptides are known to regulate a different types of cellular processes, such as transport and localization, hierarchical organization and oligomerization, including facilitation of proper protein folding, and regulation of protein activity-function. Further, inclusion of a pro-protein signal peptide can enrich for the amount of protein in certain cellular localizations. For example, inclusion of a pro-protein sequence peptide on a protein of interest can enrich for the amount of the protein of interest in the paraplasm of yeast. In the context of facilitating translocation, the effect of the pre-protein signal peptide, pro-protein signal peptide, or combination thereof as described herein is target dependent. While not wishing to be bound by theory, in some embodiments a pre-protein signal peptide without the pro-protein signal peptide will facilitate more efficient translocation and secretion. In some embodiments, a pro-protein signal peptide without the pre-protein signal peptide will facilitate more efficient translocation and secretion. In some embodiments, inclusion of both the pre and pro-protein signal peptides will facilitate more efficient secretion.

The chemical makeup of a peptide will be described herein by a series of amino acid single letter abbreviations or an “amino acid sequence/s” or “sequence/s,” which are conventional and known to those in the art. While reference sequences will be explicitly disclosed, in any aspect and embodiment, a reference sequence may be modified to include conservative amino acid substitutions, as well as variants and fragments, while maintaining the characteristics and functionality of the reference sequence.

The methods disclosed herein utilize a synthetic signal peptide to increase extracellular secretion of a payload protein by a yeast. As used herein, a “synthetic signal peptide” refers to a signal peptide whose sequence is generated as provided for herein and that is made recombinantly. The recombinantly produced signal peptide can be referred to as a “synthetic signal peptide” or simply as a “signal peptide”. The signal peptide comprising one or more of a synthetic pre-protein (sPre) signal peptide and a synthetic pro-protein (sPro) signal peptide. As highlighted previously, the term synthetic in this context refers to a recombinantly produced pre-protein signal peptide or pro-protein signal peptide whose sequence is generated as provided for herein. Hereafter, the pre- and pro-signal peptides may be referred to as “synthetic” pre or pro-protein signal peptides, or simply as pre or pro-protein signal peptides. In embodiments where a native pre or pre-protein signal peptide is utilized or referred to, the peptide will be denoted as such. In the context of this application, the term “native” refers to a pre or pro signal peptide the sequence of which is adopted, in whole or in part, from a known pre or pro signal peptide sequence at the time of this application. In other words, the “native” signal peptides are not generated using the formulas or methods as provided for herein. However, it is to be understood that a synthetic signal peptide may comprise a synthetic pre-protein signal peptide fused with a native pro-protein signal peptide (sPre-nPro signal peptide). In another example, a synthetic signal peptide may comprise a native pre-protein signal peptide fused to a synthetic pro-protein signal peptide (nPre-sPro signal peptide). In yet another example, a synthetic signal peptide comprises a synthetic pre-protein signal peptide and no pro-protein signal peptide. Similarly, a synthetic signal peptide may comprise a synthetic pro-protein signal peptide but no pre-protein signal peptide.

A pre-protein signal peptide (synthetic or native) comprises 10 to 50 amino acids, which are appended either directly to the N-terminus of a payload protein or indirectly to the N-terminus of a payload protein, with one or more of a Kex protease (KR) site, Ste13 cleavage site, and spacer there between.

A pro-protein signal peptide comprises 10 to 200 amino acids that are appended either directly to the N-terminus of a payload protein or indirectly to the N-terminus of a payload protein, with one or more of a KR site, Ste13 cleavage site, and spacer there between. Many proteins are natively expressed comprising a pro-protein signal peptide, though, as will be described, these native pro-protein signal peptides often lack the activity to generate sufficient secretion of a payload protein. The various synthetic signal peptides described herein may be used as a replacement of all or part of a native signal peptides.

A pre- and/or pro-protein signal peptide, whether synthetic or native, may be appended to an adjacent amino acid via a bond to the N-terminal amino acid of the adjacent amino acid, for example, by a peptide bond, a dipeptide spacer, or a membrane-associating/lipidophilic alpha-helical peptide signal peptide (e.g., MISTIC, represented by the amino acid sequence

FCTFFEKHHRKWDILLEKSTGVMEA or SEQ ID NO. 26).

As used herein, “hydropathy index” or “HP index” refers to the “intrinsic” hydrophobicity/hydrophilicity of amino acid side chains in peptides/proteins as defined in Kovacs J M, Mant C T, Hodges R S. Determination of intrinsic hydrophilicity/hydrophobicity of amino acid side chains in peptides in the absence of nearest-neighbor or conformational effects. Biopolymers. 2006; 84(3):283-97. doi: 10.1002/bip.20417. PMID: 16315143; PMCID: PMC2744689, which is hereby incorporated by reference in its entirety. Hydrophobicity/hydrophilicity values were determined via a synthetic peptide wherein the HP index value is calculated as the difference in RP-HPLC retention time between amino acid X at the i position and amino acid Gly at the i+1 position. Thus, amino acids that are more hydrophobic than glycine have a positive HP index value and amino acids that are more hydrophilic than glycine have a negative HP index value, wherein glycine would have a 0 value. See Table 1 below, values which correspond to the values utilized for the present application.

TABLE 1

| Amino Acid | pH 5, 10 mM PO₄ |
|---|---|
| Substitution | Buffer Δt_{R(Gly)} |
| Trp (W) | 33.2 |
| Phe (F) | 30.1 |
| Leu (L) | 24.1 |
| Ile (I) | 22.2 |
| Met (M) | 16.4 |
| Tyr (Y) | 15.2 |
| Val (V) | 14.0 |
| Pro (P) | 9.4 |
| Cys (C) | 7.9 |
| Ala (A) | 3.3 |
| Glu (E) | −0.5 |
| Thr (T) | 2.8 |
| Asp (D) | −1.0 |
| Gln (Q) | 0.6 |
| Ser (S) | 0.0 |
| Asn (N) | 0.0 |
| Gly (G) | 0.0 |
| Arg (R) | −3.7 |
| His (H) | −5.1 |
| Lys (K) | −3.7 |

As used herein “helicity” refers to the nonpolar phase helical propensity of each guest “X” residue in an experimental KKAAAXAAAAAXAAWAAXAAAKKKK (SEQ ID NO. 84)—amide peptide, as outlined in Deber C M, Wang C, Liu L P, Prior A S, Agrawal S, Muskat B L, Cuticchia A J. TM Finder: a prediction program for transmembrane protein segments using a combination of hydrophobicity and nonpolar phase helicity scales. Protein Sci. 2001 January; 10(1):212-9. doi: 10.1110/ps.30301. PMID: 11266608; PMCID: PMC2249854, which is hereby incorporated by reference in its entirety. Helicity values for each amino acid are in Table 2 below.

TABLE 2

| Amino Acid | Helicity |
|---|---|
| F | 1.26 |
| W | 1.07 |
| L | 1.28 |
| I | 1.29 |
| M | 1.22 |
| V | 1.27 |
| C | 0.79 |
| Y | 1.11 |
| A | 1.24 |
| T | 1.09 |
| E | 0.85 |
| D | 0.89 |
| Q | 0.96 |
| R | 0.95 |
| S | 1.00 |
| G | 1.15 |
| N | 0.94 |
| H | 0.97 |
| P | 0.57 |
| K | 0.88 |

As used herein, “payload protein” or “protein of interest” refers to the protein that will be generated by the host and chaperoned through the secretory pathway into the extracellular space, facilitated by the presence of a synthetic signal peptide. Upon secretion into the extracellular space, all, some, or none of the synthetic signal peptide may be fused to the payload protein. Optionally, a payload protein still being attached partially or fully to the synthetic signal peptide may be further processed, for example, to remove the remaining signal peptide. A payload protein may be any protein known or yet to be known, for example, an enzyme, enzyme inhibitor, growth factor, hormone, antibody, antigen, vaccine, a therapeutic agent, or any combination thereof. More specific examples follow herein below.

The compositions disclosed herein may be provided to a subject in a variety of ways through administration of the composition to the subject. As used herein, administer or administration means to provide or the providing of a composition to a subject. Oral administration, as used herein, refers to delivery of an active agent through the mouth. Topical administration, as used herein, refers to the delivery of an active agent to a body surface, such as the skin, a mucosal membrane (e.g., nasal membrane, vaginal membrane, buccal membrane, or the like).

A payload protein secreted by the various genetically modified yeast disclosed herein, which are interchangeably referred to as “engineered yeast”, may be provided to a subject in a pharmaceutical composition. Additionally or alternatively, the engineered yeast itself may be provided to a subject in a pharmaceutical composition.

The various compositions disclosed herein may be useful in treating a number of diseases, for example, cancer. As used herein, cancer refers to a condition characterized by unregulated cell growth. Examples of cancer include, but are not limited to, squamous cell cancer, small-cell lung cancer, non-small cell lung cancer, lung adenocarcinoma, lung squamous cell carcinoma, gastrointestinal cancer, Hodgkin's and non-Hodgkin's lymphoma, pancreatic cancer, glioblastoma, cervical cancer, colon cancer, colorectal cancer, endometrial or uterine carcinoma, kidney cancer such as renal cell carcinoma and Wilms' tumors, basal cell carcinoma, melanoma, prostate cancer, and esophageal cancer. In some embodiments, the diseases or conditions may include, but is not limited to, an infection, an autoimmune disease, enzymatic deficiencies (including primary (congenital) enzymatic deficiency and enzymatic deficiencies secondary to functional gut disorders), diabetes, obesity, metabolic disorders, intestinal bacterial overgrowth, enteric infection, bacterial vaginosis, short bowel syndrome, inflammatory bowel disease, irritable bowel syndrome, small bowel syndrome, Celiac disease, gluten intolerance, colitis, peptic ulcer, gastritis, polyps, hemorrhoids, cirrhosis, or a cancer

The various compositions disclosed herein may comprise one or more drugs, biologics, or active agents, which are used interchangeably herein and refer to a chemical substance or compound that induces a desired pharmacological or physiological effect, and includes agents that are therapeutically effective, prophylactically effective, or cosmetically effective. “Drug,” “biologic,” and “active agent” include any pharmaceutically acceptable, pharmacologically active derivatives and analogs of those drugs, biologics, and active agents specifically mentioned herein, including, but not limited to, salts, esters, amides, prodrugs, active metabolites, inclusion complexes, analogs, and the like. Suitable drugs, biologics, and active agents may include, but are not limited to, alcohol deterrents; amino acids; ammonia detoxicants; anabolic agents; analeptic agents; analgesic agents; androgenic agents; anesthetic agents; anorectic compounds; anorexic agents; antagonists; anti-allergic agents; anti-amebic agents; anti-anemic agents; anti-anginal agents; anti-anxiety agents; anti-arthritic agents; anti-atherosclerotic agents; anti-bacterial agents; anti-cancer agents, including antineoplastic drugs, and anti-cancer supplementary potentiating agents; anticholinergics; anticholelithogenic agents; anti-coagulants; anti-coccidal agents; anti-convulsants; anti-depressants; anti-diabetic agents; anti-diarrheals; anti-diuretics; antidotes; anti-dyskinetics agents; anti-emetic agents; anti-epileptic agents; anti-estrogen agents; anti-fibrinolytic agents; anti-fungal agents; anti-glaucoma agents; anti-hemophilic agents; anti-hemorrhagic agents; antihistamines; anti-hyperlipidemic agents; anti-hyperlipoproteinemic agents; antihypertensive agents; anti-hypotensives; anti-infective agents such as antibiotics and antiviral agents; anti-inflammatory agents, both steroidal and non-steroidal; anti-keratinizing agents; anti-malarial agents; antimicrobial agents; anti-migraine agents; anti-mitotic agents; anti-mycotic agents; antinauseants; antineoplastic agents; anti-neutropenic agents; anti-obsessional agents; anti-parasitic agents; antiparkinsonism drugs; anti-pneumocystic agents; anti-proliferative agents; anti-prostatic hypertrophy drugs; anti-protozoal agents; antipruritics; anti-psoriatic agents; antipsychotics; antipyretics; antispasmodics; anti-rheumatic agents; anti-schistosomal agents; anti-seborrheic agents; anti-spasmodic agents; anti-thrombotic agents; anti-tubercular agents; antitussive agents; anti-ulcerative agents; anti-urolithic agents; antiviral agents; GERD medications, anxiolytics; appetite suppressants; attention deficit disorder (ADD) and attention deficit hyperactivity disorder (ADHD) drugs; bacteriostatic and bactericidal agents; benign prostatic hyperplasia therapy agents; blood glucose regulators; bone resorption inhibitors; bronchodilators; carbonic anhydrase inhibitors; cardiovascular preparations including anti-anginal agents, anti-arrhythmic agents, beta-blockers, calcium channel blockers, cardiac depressants, cardiovascular agents, cardioprotectants, and cardiotonic agents; central nervous system (CNS) agents; central nervous system stimulants; choleretic agents; cholinergic agents; cholinergic agonists; cholinesterase deactivators; coccidiostat agents; cognition adjuvants and cognition enhancers; cough and cold preparations, including decongestants; depressants; diagnostic aids; diuretics; dopaminergic agents; ectoparasiticides; emetic agents; enzymes which inhibit the formation of plaque, calculus or dental caries; enzyme inhibitors; estrogens; fibrinolytic agents; fluoride anticavity/antidecay agents; free oxygen radical scavengers; gastrointestinal motility agents; genetic materials; glucocorticoids; gonad-stimulating principles; hemostatic agents; herbal remedies; histamine H2 receptor antagonists; hormones; hormonolytics; hypnotics; hypocholesterolemic agents; hypoglycemic agents; hypolipidemic agents; hypotensive agents; immunizing agents; immunomodulators; immunoregulators; immunostimulants; immunosuppressants; impotence therapy adjuncts; inhibitors; keratolytic agents; leukotriene inhibitors; liver disorder treatments; metal chelators such as ethylenediaminetetraacetic acid, tetrasodium salt; mitotic inhibitors; mood regulators; mucolytics; mucosal protective agents; muscle relaxants; mydriatic agents; narcotic antagonists; neuroleptic agents; neuromuscular blocking agents; neuroprotective agents; nicotine; NMDA antagonists; non-hormonal sterol derivatives; nutritional agents, such as vitamins, essential amino acids and fatty acids; ophthalmic drugs such as antiglaucoma agents; oxytocic agents; pain relieving agents; parasympatholytics; peptide drugs; plasminogen activators; platelet activating factor antagonists; platelet aggregation inhibitors; post-stroke and post-head trauma treatments; potentiators; progestins; prostaglandins; prostate growth inhibitors; proteolytic enzymes as wound cleansing agents; prothyrotropin agents; psychostimulants; psychotropic agents; radioactive agents; regulators; relaxants; repartitioning agents; scabicides; sclerosing agents; sedatives; sedative-hypnotic agents; selective adenosine A1 antagonists; serotonin antagonists; serotonin inhibitors; serotonin receptor antagonists; steroids, including progestogens, estrogens, corticosteroids, androgens and anabolic agents; smoking cessation agents; stimulants; suppressants; sympathomimetics; synergists; thyroid hormones; thyroid inhibitors; thyromimetic agents; tranquilizers; tooth desensitizing agents; tooth whitening agents such as peroxides, metal chlorites, perborates, percarbonates, peroxyacids, and combinations thereof; unstable angina agents; uricosuric agents; vasoconstrictors; vasodilators including general coronary, peripheral and cerebral; vulnerary agents; wound healing agents; xanthine oxidase inhibitors; and the like.

Antibiotic refers to a chemical substance capable of treating bacterial infections by inhibiting the growth of, or by destroying existing colonies of bacteria and other microorganisms.

Anti-inflammatory refers to an active agent that reduces inflammation and swelling.

Chemotherapeutic agent refers to a chemical agent with therapeutic usefulness in the treatment of diseases characterized by abnormal cell growth. Such diseases include tumors, neoplasms, and cancer. In one example, a chemotherapeutic agent is a radioactive compound. In one example, a chemotherapeutic agent is a biologic, such as a monoclonal antibody. Chemotherapy refers to use of a chemotherapeutic agent.

Radiation therapy refers to use of directed gamma rays or beta rays to induce sufficient damage to a cell so as to limit its ability to function normally or to destroy the cell altogether.

The various compositions disclosed herein may comprise an effective amount of a drug, biologic, or active agent. Effective amount refers to an amount of a drug, biologic, or active agent (alone or with one or more other active agents) sufficient to induce a desired response, such as to prevent, treat, reduce and/or ameliorate a condition. An effective amount of an active agent, alone or with one or more other active agents, can be determined in many different ways, such as assaying for a reduction in of one or more signs or symptoms associated with the condition in the subject or measuring the level of one or more molecules associated with the condition to be treated.

The various compositions disclosed herein may comprise various pharmaceutically acceptable excipients. As used herein, a pH adjuster or modifier refers to a compound or buffer used to achieve desired pH control in a formulation. Exemplary pH modifiers include acids (e.g., acetic acid, adipic acid, carbonic acid, citric acid, fumaric acid, phosphoric acid, sorbic acid, succinic acid, tartaric acid), bases (e.g., magnesium oxide, tribasic potassium phosphate), and pharmaceutically acceptable salts thereof.

Pharmaceutically acceptable carriers useful in this disclosure are those conventionally known in the art. The nature of the carrier can depend on the particular mode of administration being employed. For instance, oral applications usually include pharmaceutically and physiologically acceptable fluids such as water, physiological saline, balanced salt solutions, aqueous dextrose, glycerol, or the like, as a vehicle. In addition to biologically-neutral carriers, oral compositions may also contain auxiliary substances, such as wetting or emulsifying agents, preservatives, and pH buffering agents, and the like.

Antioxidant refers to a compound that inhibits oxidation or reactions promoted by oxygen or peroxides.

Mucoadhesive refers to a substance that strongly attaches to mucosa upon hydration without any additional adhesive material, and remains adhered to the tissue in vivo.

### Synthetic Signal Peptides

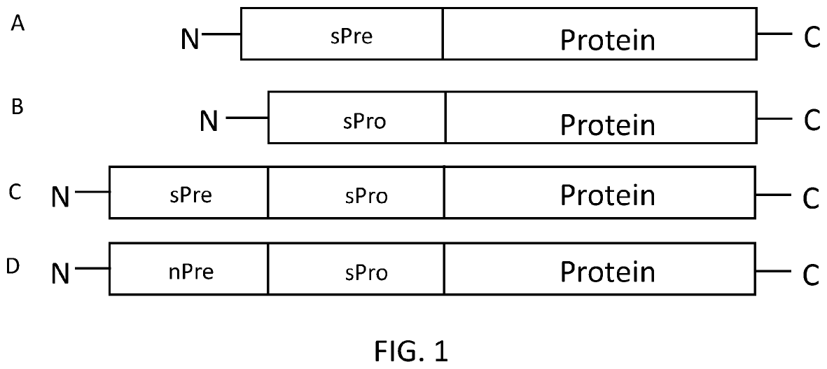

In some embodiments, synthetic signal peptides that increase secretion of a payload protein from yeast are provided. In some embodiments, the synthetic signal peptide, as described above, comprises one or more of a synthetic pre-protein signal peptide and pro-protein signal peptide. In any embodiment, a native pre- or pro-protein signal peptide may be combined with a synthetic signal peptide, provided at least one of the pre- and pro-protein signal peptide is synthetic. In some embodiments, recombinant polypeptides are provided comprising a synthetic signal peptide and a payload protein, wherein the synthetic signal peptide is fused, either directly or indirectly, to the payload protein. In some embodiments, the synthetic signal peptide is fused directly to the protein of interest. In some embodiments, the synthetic signal peptide and protein of interest are connected via a peptide linker. Suitable peptide linkers are known in the art and any such linker may be utilized. In some embodiments, the linker is a flexible peptide linker. In some embodiments, the linker is a non-cleavable peptide linker. In some embodiments the linker is a cleavable peptide linker. In some embodiments, the recombinant polypeptide comprises a synthetic pre-protein signal peptide and a payload protein. For example, FIG. 1 depicts a construct that represents a recombinant polypeptide comprising a synthetic signal peptide appended to the N-terminus of a payload protein wherein the synthetic signal peptide comprises only a synthetic pre-protein signal peptide (sPre signal peptide, labeled A). In some embodiments, the recombinant polypeptide comprises a synthetic pro-protein signal peptide and a payload protein. For example, FIG. 1 depicts a construct that represents a recombinant polypeptide comprising a synthetic signal peptide appended to the N-terminus of a payload protein wherein the synthetic signal peptide comprises a synthetic pro-protein signal peptide only (sPro signal peptide, labeled B). In some embodiments, the recombinant polypeptide comprises a synthetic pre-protein signal peptide, a synthetic pro-protein signal peptide, and a payload protein. For example, FIG. 1 depicts a construct that represents a recombinant polypeptide comprising a synthetic signal peptide appended to the N-terminus of a payload protein wherein the synthetic signal peptide comprises both of a synthetic pre-protein signal peptide and a synthetic pro-protein signal peptide (sPre-sPro signal peptide, labeled C). The pre-protein signal peptide is appended to the N-terminus of the pro-protein signal peptide, which is appended to the N-terminus of the payload protein. In some embodiments, the recombinant polypeptide comprises a native pre-protein signal peptide, a synthetic pro-protein signal peptide, and a payload protein. For example, FIG. 1 depicts a construct that represents a recombinant polypeptide comprising a synthetic signal peptide comprising a native pre-protein signal peptide fused to a synthetic pro-protein signal peptide (nPre-sPro signal peptide, labeled D). In some embodiments, the recombinant polypeptide comprises a synthetic pre-protein signal peptide, a native pro-protein signal peptide, and a payload protein.

Table 3 below lists various amino acid sequences that will be referred to herein. In Table 3, amino acids contained within parentheses are optional. It is to be understood that when multiple amino acids are contained within parentheses, any one of the amino acids can be added or excluded without the addition of the other. The sequences EEGEPK (SEQ ID NO. 78) and DVVYPK (SEQ ID NO. 79) are spacers and DKREEGPK (SEQ ID NO. 80), KREEGPK (SEQ ID NO. 81), DKREKRE (SEQ ID NO. 82), and DKR (SEQ ID NO. 83) are Kex protease sites.

TABLE 3

| SEQ | | Pre- or |
|---|---|---|
| ID | | Pro- |
| NO. | Amino Acid Sequence | Protein |
| 1 | MKLSSLLLLLLLLLSSLVLAA | Pre |
| 2 | MRFPSIFTAVLFAASSALA | Pre |
| | | (∝- |
| | | MF, S. |
| | | cerevisiae) |
| 3 | MFSPILSLEIILALATLQSVFAR | Pre |
| 4 | MKLSTLLLTLLLLLLALVLA(AS) | Pre |
| 5 | MLKLLLLILLLLLLVSLVLAAS | Pre |
| 6 | MKLLLLLLLLLLLLLLLALVLA(AS) | Pre |
| 7 | MKLLLLLLSLVLAAS | Pre |
| 8 | MKLSSLLLALLLALA | Pre |
| 9 | MKLSSLLLALLLALASLALA(AP) | Pre |
| 10 | MKLSSLLLALLLALASLALAAP(K) | Pre |
| 11 | MKLKTVRSAVLSSLFASQVLG | Pre |
| 12 | MKFLSLLLALVAALALALALAAP | Pre |
| 13 | MLLQAFLFLLAGFAAKISA | Pre |
| 14 | MKFKLTLLAALLALAALVLAAS | Pre |
| 15 | MKLSSILLLLALLALVLAAS | Pre |
| 16 | MKLLSLLALLLLLASLVLAAS | Pre |
| 17 | APVNTTTEDETAQIPAEAVIGYSDLEGDFDVAV | Pro |
| | LPFSNSTNNGLLFINTTIASIAAKEEGVSLEKRE | |
| 18 | SLALAAPVNTTTEDETAQIPAEAVIGYSDLEGDEDVA | Pro (∝- |
| | VLPFSNSTNNGLLFINTTIASIAAKEEGVSL(DKREEGEPK) | MF) |
| 19 | QPIDDTESQTTSVNLMADDTESAFATQ | Pro |
| | TNSGGLDVVGLISMA(KR)(EEGEPK) | (TA57) |
| 20 | IPLVANVSFNSDNGSQWLY(KREEGEPK) | Pro |
| 21 | IPLVANVSFNSDNGSQWLY(KRDVVYPK) | Pro |
| 22 | EPWSTLTVTRSTYDEITDTDYNSTGIAVNPYTVSASRHKRDV | Pro |
| 23 | STLTPSVVFIGGGLTEETTFGIRHKRDV | Pro |
| 24 | DPWSTTTSIYSLGGTTSYVSEFGLSISDETVTEMKSRHKRDV | Pro |
| 25 | APVNTTTEDETAQIPAEAVIGYSDLEGDFDVAVLPFS | Pro |
| | NSTNNGLLFINTTIASIAAKEEGVSL(DKREEGEPK) | |
| 26 | FCTFFEKHHRKWDILLEKSTGVMEA | MISTIC |
| 27 | APVNTTTEDETAQIPAEAVIGYSDLEGDFDVAVL | Pro (∝- |
| | PFSNSTNNGLLFINTTIASIAAKEEGVSL(DKR) or (DEKRE) | MF, S. |
| | | cerevisiae) |
| 28 | MKFSTILAASTALISVVMA | Pre (∝- |
| | | MF, K. |
| | | lactis) |
| 29 | APVSTETDIDDLPISVPEEALIGFIDLTGDEVSL | Pro (∝- |
| | LPVNNGTHTGILFLNTTIAEAAFADKDDLE | MF, K. |
| | | lactis) |
| 30 | MFSPILSLEIILALATLQSVFAR | pre- |
| | | pro-PHO1 |
| 31 | MKSSLLLLALLALAALASA(AP) | Pre |
| 32 | MKSSLLLLLLALASLALA(AP) | Pre |
| 33 | MKSSSLLLLALLALLAALASA(AP) | Pre |
| 34 | EARDSWASPGGQSTRADDAINVAG TGGSILRPAKFSTLDSRRSKRSSE | Pro |
| 35 | PSVRPPVASRDYSHSARWSTKKRSRR | Pro |
| 36 | LHVFSTDYLLIYIVGDLPRRVKRSV | Pro |
| 37 | SLSPVDNILLLQLLGTAAAVLIGASKNDGGVTRVED | Pro |
| | RSYSEILFSLNPLSDTRLRLKHVVAVAGGKASSVTD | |
| | YYLSLSRNVPEGVWYMATSIDGEIAWLGLIADVLRG | |
| | VFLLGEAARESV | |
| 38 | SYSRVSNAAGKSRGDCRSRLFPDGTKSPPSASPRREARR | Pro |
| | DSLHPVLTSSAESGSGSYMSSNSARDLLRSGAKNHL | |
| | GAPNNDQVKFGLATRTRKRAE | |
| 55 | MRSLLILVLCFLPLAALG | Pre |
| 56 | APVSTETDIDDLPISVPEEALIGFIDLTGDEVSLLPVNNGTHTGILFLNTTI | Pro |
| | AEAAFADKDDLEKR | |
| 57 | APIPLVANVSFNSDNGSQWLYKRDVVYPK | Pro |
| 58 | APIPLVANVSFNSDNGSQWLYKREEGEPK | Pro |
| 70 | MRSLSLALLLLLALLASLALAAP | Pre |
| 71 | MRLSLSLLLLLLALLASLALAAP | Pre |
| 72 | MRLSSLLLGLLLALAASLALAAP | Pre |
| 73 | MRLSLLLALLALLALASLALAAP | Pre |
| 74 | ASSGRSPTITGQVSTLSSTDGTLPTSFTSGSAAGTISSTLPSNVTSTLGTID | Pro |
| | LSPNGSADSSSKRST | |
| 75 | SPTTSPSTTASLVSTSVTSSVTLTSTDVTTSEDTTGFVLPDSGTSSGTAD | Pro |
| | ALEAYSIGITSSSAVVDSKKRDA | |

In addition to the Kex protease sites recited in the above examples, the pre-protein signal peptides and pro-protein signal peptides of the present disclosure may also optionally contain a KEX2 cleavage site, as given by the amino acid sequence NVISKR (SEQ ID NO. 68), or the amino acid sequence SDVTKR (SEQ ID NO. 69). In any embodiment, the sequence of SEQ ID NO. 68 can be appended to the C-terminus or N-terminus of any pre- or pro-protein signal peptide as provided for herein. Accordingly, in some embodiments, the pre-protein signal peptide is as provided. In some embodiments, the pro-protein signal peptide is as provided. In any embodiment, the sequence of SEQ ID NO. 69 can be appended to the C-terminus or N-terminus of any pre- or pro-protein signal peptide as provided for herein. Accordingly, in some embodiments, the pre-protein signal peptide is as provided. In some embodiments, the pro-protein signal peptide is as provided.

In some embodiments, the KEX2 cleavage site can be represented by the following formula:

X₄X₃X₂X₁B₁B₂ (Formula XII)

wherein i) X₁, X₂, and X₃ are not G, ii) X₁ is not 5, if X₂ and X₃ are G, X₄ is A, or X₅ is 5, iii) X₄ is not T, if X₃ is A and X₂ is 5; or iv) X₁ is not D; and wherein B₁ and B₂ are each, independently, basic amino acids. The details of Formula XII are described in U.S. Pat. No. 8,936,917, which is hereby incorporated by reference in its entirety. Accordingly, in any embodiment, the sequence of Formula XII can be appended to the C-terminus or N-terminus of any pre- or pro-protein signal peptide as provided for herein. In some embodiments, the pre-protein signal peptide is as provided. In some embodiments, the pro-protein signal peptide is as provided.

Any synthetic pre-protein or pro-protein signal peptide may be combined with some or all of a known signal peptide. Examples of known signal peptides that may be combined with any of SEQ ID Nos 1-25, 31-38, 55-58, and 70-75 in Table 3 to generate a synthetic signal peptide include, but are not limited to, HSp150, PH05, SUC2, KILM1, GGP1, SUN, PLB, CRH, EXG, AGA2, HAS pre-pro, PIR1, XPR2 pre, XPR2 pre-pro, pGKL, SCW, and DSE.

One who is skilled in the art will be able to develop a nucleic acid that encodes for the expression of any one of SEQ ID NOs. 1-38, 55-58, and 70-75. Table 4 below provides example nucleotide sequences that may be used to generate the synthetic peptides described in Table 3. It is to be understood that the nucleic acid sequences provided in Table 4 are exemplary and are not meant to be limiting in any way. Due to the degenerate nature of codons, other nucleic acid molecules can be used. In some embodiments, the nucleic acid molecule is codon optimized for expression in a bacterial system. In some embodiments, the nucleic acid molecule is codon optimized for expression in a eukaryotic system or cell.

TABLE 4

| SEQ | Amino | |
|---|---|---|
| ID | Acid SEQ | |
| NO. | ID NO. | Nucleotide Sequence |
| 39 | 1 | ATGAA ATTGT CTTCT TTGTT GTTGT TGTTG TTGTT GTTGT |
| | | TGTCT TCTTT GGTTT TGCT |
| 40 | 2 | ATGTT CTCTC CAATT TTGTC CTTGG AAATT ATTTT |
| | | AGCTT TGGCT ACTTT GCAAT CTGTC TTCGC TCGA |
| 41 | 3 | ATGAA ATTGT CTACT CTGTT GTTGA CTTTG TTGTT GTTGT |
| | | TGTTG GCTTT GGTTT TGGCT GCTTC T |
| 42 | 4 | ATGTT GAAAT TGTTG CTGTT GATTT TGTTG TTGTT GCTTT |
| | | TGGTT TCTTT GGTTT TGGCT GCTTC T |
| 43 | 5 | ATGAA ATTGT TACTG CTTTT ACTTC TTTTG CTGTT ATTGT |
| | | TGCTT TTGCT GGCTT TGGTT TTGGC TGCTT CT |
| 44 | 6 | ATGAA ATTGT TGTTG TTGTT GTTGT CTTTG GTTTT GGCTG |
| | | CTTCT |
| 45 | 7 | ATGTT CTCTC CAATT TTGTC CTTGG AAATT ATTTT |
| | | AGCTT TGGCT ACTTT GCAAT CTGTC TTCGC TCGA |
| 46 | 8 | ATGAA GTTAT CTTCT TTATT GCTGG CTCTG CTTCT AGCCT |
| | | TGGCG |
| 47 | 9 | ATGAA GTTAT CTTCT TTATT GCTGG CTCTG CTTCT AGCCT |
| | | TGGCG TCTCT AGCGC TGGCC |
| 48 | 10 | ATGAA GTTAT CTTCT TTATT GCTGG CTCTG CTTCT AGCCT |
| | | TGGCG TCTCT AGCGC TGGCC GCACC AAAG |
| 49 | 11 | ATGAA ATTGA AAACT GTTAG ATCTG CTGTT TTGTC TTCTT |
| | | TGTTT GCTTC TCAAG TTTTG GGT |
| 50 | 17 | GCTCC AGTCA ACACT ACAAC AGAAG ATGAA ACGGC |
| | | ACAAA TTCCG GCTGA AGCTG TCATC GGTTA CTCAG ATTTA |
| | | GAAGG GGATT TCGAT GTTGC TGTTT TGCCA TTTTC CAACA |
| | | GCACA AATAA CGGGT TATTG TTTAT AAATA CTACT ATTGC |
| | | CAGCA TTGCT GCTAA AGAAG AAGGG GTATC TCTCG AGAAA |
| | | AGAGA G |
| 51 | 18 | GCTCC AGTTA ATACT ACTAC TGAAG ATGAA ACTGC TCAAA |
| | | TTCCA GCTGA AGCTG TTATT GGTTA TTCTG ATTTG GAGGG |
| | | TGACT TTGAT GTTGC TGTTT TGCCA TTTTC TAACT CTACT |
| | | AACAA CGGTT TGCTA TTCAT CAACA CTACT |
| 52 | 19 | CAACC AATTG ATGAT ACTGA ATCTC AAACT ACTTC TGTTA |
| | | ATTTG ATGGC TGATG ATACT GAATC TGCTT TTGCT ACTCA |
| | | AACTA ATTCT GGTGG TTTGG ATGTT GTTGG TTTGA TTTCT |
| | | ATGGC TAAAA GAGAA GAAGG TGAAC CAAAA |
| 53 | 20 | GCTCC AATTC CATTG GTTGC TAATG TTTCT TTTAA TTCTG |
| | | ATAAT GGTTC TCAAT GGTTG TATAA AAGAG AAGAA GGTGA |
| | | ACCAA AA |
| 54 | 21 | GCTCC AATTC CATTG GTTGC TAATG TTTCT TTTAA TTCTG |
| | | ATAAT GGTTC TCAAT GGTTG TATAA AAGAG ATGTT GTTTA |
| | | TCCAA AA |

The synthetic signal peptides disclosed herein are optimized for use in yeast and can be used to induce expression of any protein. Particular examples of suitable yeast species are provided herein below to exemplify the particular synthetic signal peptides that have been developed.

As noted above, Table 3 discloses amino acid sequences, however, in any aspect and embodiment, any of the sequences in Table 3 may be modified with conservative amino acid substitutions to produce active variants that maintain the characteristics and functionality of the primary sequence. These conservative amino acid substitutions can be generally described by the Formulas below, which encapsulate the consensus sequence as well as the variant sequences. The various Formulas detailing the variant sequences will now be described.

In some embodiments, a pre-protein signal peptide is provided. In some embodiments, the pre-protein signal peptide comprises an amino acid sequence selected from the group consisting of Formula I, Formula II, Formula III, Formula IV, Formula V, Formula IX, and Formula XIII.

### Variants of SEQ ID NO. 1 (Formula I)

In some embodiments, the pre-protein signal peptide comprises an amino acid sequence represented by:

A₁-(A₂)_{w}-A₃-(A₄)ₓ-(A₅)_{y}-(A₆)-(A₇)-(A₈)-(A₉)-(A₁₀)-(A₁₁)_{z} (Formula I)

wherein:
w and x are each, independently, 1, 2, 3, 4, or 5;
y is 2, 3, 4, 5, 6, 7, 8, 9, 10, 11, 12, 13, 14, 15, 16, 17, 18, 19, or 20; and
z is 1, 2, or 3;
   wherein
A₁ is methionine
   each A₂ is, independently, a neutral or positively-charged amino acid with a hydropathy index less than about 1;
each A₃, A₅, A₈, and A₁₀ is, independently, an amino acid with a hydropathy index greater than −1, excluding W and C;
each A₄ is, independently, a basic or neutral amino acid, excluding P, W, M, and C;
each A₆ is, independently, an amino acid with a hydropathy index greater than −1, excluding W, M, and C;
each A₇ is, independently, a non-aromatic amino acid with a hydropathy index less than about 1.9 and an isoelectric point of about 5.4 to 7.5 (inclusive), excluding P;
each A₉ is, independently, an amino acid with a hydropathy index greater than about −1.3; and
each A₁₁ is, independently, a neutral amino acid with a molecular weight less than about 133 g/mol.

In some embodiments, w is 1. In some embodiments w is 2. In some embodiments, w is 3. In some embodiments, w is 4. In some embodiments, w is 5. In some embodiments, x is 1. In some embodiments, x is 2. In some embodiments, x is 3. In some embodiments, x is 4. In some embodiments, x is 5. In some embodiments, y may be an integer selected from 2-18, 4-16, 6-14, 8-12, 7-11, and 8-10. In some embodiments, y is 2. In some embodiments, y is 3. In some embodiments, y is 4, 5, 6, 7, 8, 9, 10, 11, 12, 13, 14, 15, 16, 17, 18, 19, or 20. In some embodiments, z is 1. In some embodiments, z is 2. In some embodiments, z is 3. It is to be understood that the values of w, x, y, and z are each independently selected, and the value of any variable w, x, y, or z is independent of the values selected for the other variables. In some embodiments, each A₃, A₅, A₈, and A₁₀ is each, independently, an amino acid selected from the group consisting of A, G, I, L, M, F, S, T, V, P, E, Y, Q, and N. In some embodiments, each A₃, A₅, A₈, and A₁₀ is each, independently, an amino acid selected from the group consisting of L, V, A, and I. In some embodiments A₃ is each an amino acid selected from the group consisting of A, G, I, L, M, F, S, T, V, P, E, Y, Q, and N. In some embodiments, A₃ is an amino acid selected from the group consisting of L, V, A, and I. In some embodiments A₅ is each an amino acid selected from the group consisting of A, G, I, L, M, F, S, T, V, P, E, Y, Q, and N. In some embodiments, A₅ is an amino acid selected from the group consisting of L, V, A, and I. In some embodiments A₈ is each an amino acid selected from the group consisting of A, G, I, L, M, F, S, T, V, P, E, Y, Q, and N. In some embodiments A₈ is an amino acid selected from the group consisting of L, V, A, and I. In some embodiments A₁₀ is each an amino acid selected from the group consisting of A, G, I, L, M, F, S, T, V, P, E, Y, Q, and N. In some embodiments A₁₀ is an amino acid selected from the group consisting of L, V, A, and I. In some embodiments, each A₁₁ is, independently, an amino acid selected from the group consisting of N, S, T, C, A, V, G, I, L, and P. In some embodiments, each A₁₁ is, independently, an amino acid selected from the group consisting of A, L, and G. In some embodiments, each A₂ is, independently, an amino acid selected from the group consisting of K, R, H and Q. In embodiments where any one of w, x, y, and z are an integer greater than 1, each amino acid in the group described by the w, x, y, and z are independently chosen from the disclosed group of amino acids and therefore may be the same or different. For example, for (A₂)_{w} wherein w is 3, this grouping expands to A₂A₂A₂ where each A₂ is, independently, a neutral or positively-charged amino acid with a hydropathy index less than about 1. This meaning, unless explicitly indicated otherwise, expands to all further formulas disclosed herein and below.

In some embodiments, the sequence of SEQ ID NO. 1 can be derived from Formula I as follows: w is 1, x is 2, y is 9, and z is 2; A₁ is methionine; A₂ is K; A₃ is L; both the first and second instances of A₄ are S; all 9 instances of A₅ are L; A₆ is S; A₇ is S; A₈ is L; A₉ is V; A₁₀ is L; and both instances of A₁₁ are A.

### Variants of SEQ ID NOs. 4-7 (Formula II)

In certain embodiments, the pre-protein signal peptide comprises an amino acid sequence represented by:

B₁-(B₂)ᵤ-(B₃)ᵥ-(B₄)_{w}-(B₅)ₓ-(B₆)_{y}-(B₇)-(B₈)-(B₉)-(B₁₀)-(B₁₁)_{z} (Formula II)

wherein:
u and w are each, independently, 0, 1, 2, or 3;
v and z are each, independently, 1, 2, or 3;
x is 0, 1, or 2; and
y is 2, 3, 4, 5, 6, 7, 8, 9, 10, 11, 12, 13, 14, 15, 16, 17, 18, 19, or 20;
   wherein:
B₁ is methionine;
each B₂, B₄, B₆, B₈ and B₁₀ is each, independently, an amino acid with a hydropathy index of greater than about −1, excluding W and C;
each B₃ is, independently, a positively-charged or polar amino acid with a hydropathy index less than about 1;
each B₅ is, independently, a polar amino acid with a hydropathy index greater than about −5 and less than −0.5, or an amino acid with an isoelectric point between about 5 and 11 excluding P, W, M, and C;
each B₇ and B₁₁ is each, independently, a neutral amino acid with a molecular weight less than about 133 g/mol; and
B₉ is an amino acid with a hydropathy index greater than about −1.3.

In some embodiments, u is 0. In some embodiments, u is 1. In some embodiments, u is 2. In some embodiments, u is 3. In some embodiments, w is 0. In some embodiments, w is 1. In some embodiments, w is 2. In some embodiments, w is 3. In some embodiments, v is 1. In some embodiments, v is 2. In some embodiments, v is 3. In some embodiments, z is 1. In some embodiments, z is 2. In some embodiments, z is 3. In some embodiments x is 0. In some embodiments, x is 1. In some embodiments, x is 2. In some embodiments, y may be an integer selected from 2-18, 4-16, 6-14, 8-12, 7-11, and 8-10. In some embodiments, y is 2. In some embodiments, y is 3. In some embodiments, y is 4, 5, 6, 7, 8, 9, 10, 11, 12, 13, 14, 15, 16, 17, 18, 19, or 20. It is to be understood that the values of u, w, v, z, x, and y are each independently selected, and the value of any variable u, w, v, z, x, or y is independent of the values selected for the other variables. In some embodiments, each B₂, B₄, B₆, B₈, and B₁₀ is each, independently, an amino acid selected from the group consisting of A, G, I, L, M, F, S, T, V, N, Q, E, P, and Y. In some embodiments, each B₂, B₄, B₆, B₈ and B₁₀ is each, independently, an amino acid selected from the group consisting of L, V, A, F, and I. In some embodiments, each B₂ is, independently, an amino acid selected from the group consisting of A, G, I, L, M, F, S, T, V, N, Q, E, P, and Y. In some embodiments, each B₂ is, independently, an amino acid selected from the group consisting of L, V, A, F, and I. In some embodiments, each B₄ is, independently, an amino acid selected from the group consisting of A, G, I, L, M, F, S, T, V, N, Q, E, P, and Y. In some embodiments, each B₄ is, independently, an amino acid selected from the group consisting of L, V, A, F, and I. In some embodiments, each B₆ is, independently, an amino acid selected from the group consisting of A, G, I, L, M, F, S, T, V, N, Q, E, P, and Y. In some embodiments, each B₆ is, independently, an amino acid selected from the group consisting of L, V, A, F, and I. In some embodiments, B₈ is an amino acid selected from the group consisting of A, G, I, L, M, F, S, T, V, N, Q, E, P, and Y. In some embodiments, B₈ is an amino acid selected from the group consisting of L, V, A, F, and I. In some embodiments, B₁₀ is an amino acid selected from the group consisting of A, G, I, L, M, F, S, T, V, N, Q, E, P, and Y. In some embodiments, B₁₀ is an amino acid selected from the group consisting of L, V, A, F, and I. In some embodiments, each B₅ is, independently, an amino acid selected from the group consisting of K, R, E, D, G, A, V, L, I, F, S, T, Y, N, and H. In some embodiments, each B₅ is, independently, an amino acid selected from the group consisting of K, R, E, and D. In some embodiments, each B₅ is, independently, an amino acid selected from the group consisting of G, A, V, L, I, F, S, T, Y, N, K, R, and H. In some embodiments, each B₇ and B₁₁ is each, independently, an amino acid selected from the group consisting of A, S, G, and P. In some embodiments, B₇ is an amino acid selected from the group consisting of A, S, G, and P. In some embodiments, each B₁₁ is, independently, an amino acid selected from the group consisting of A, S, G, and P. In some embodiments, B₉ is an amino acid selected from the group consisting of A, C, G, I, L, M, F, S, T, W, Y, V, N, Q, D, E, and P. In some embodiments, each B₃ is each, independently, an amino acid selected from the group consisting of K, R, H and Q. In embodiments where any one of u, w, v, z, x and y are an integer greater than 1, each amino acid in the group described by the u, w, v, z, x and y are independently chosen from the disclosed group of amino acids and therefore may be the same or different, as described for herein.

In some embodiments, the sequence of SEQ ID NO. 4 can be derived from Formula II as follows: u is 0, v is 1, w is 1, x is 1, y is 11, and z is 3; B₁ is methionine; B₂ is absent; B₃ is K; B₄ is L; B₅ is S; the string of eleven (11) B₆ residues is as follows: T-L-L-L-T-L-L-L-L-L-L (SEQ ID NO: 87); B₇ is A; B₈ is L; B₉ is V; B₁₀ is L; and the string of three (3) B₁₁ residues is as follows: A-A-S.

In some embodiments, the sequence of SEQ ID NO. 5 can be derived from Formula II as follows: u is 1, v is 1, w is 1, x is 0, y is 11, and z is 3; B₁ is methionine; B₂ is L; B₃ is K; B₄ is L; B₅ is absent; the string of eleven (11) B₆ residues is as follows: L-L-L-I-L-L-L-L-L-L-V (SEQ ID NO: 88); B₇ is S; B₈ is L; B₉ is V; B₁₀ is L; and the string of three (3) B₁₁ residues is as follows: A-A-S.

In some embodiments, the sequence of SEQ ID NO. 6 can be derived from Formula II as follows: u is 0, v is 1, w is 0, x is 0, y is 15, and z is 3; B₁ is methionine; B₂ is absent; B₃ is K; B₄ is absent; B₅ is absent; all fifteen (15) B₆ residues are L; B₇ is A; B₈ is L; B₉ is V; B₁₀ is L; and the string of three (3) B₁₁ residues is as follows: A-A-S.

In some embodiments, the sequence of SEQ ID NO. 7 can be derived from Formula II as follows: u is 0, v is 1, w is 0, x is 0, y is 6, and z is 3; B₁ is methionine; B₂ is absent; B₃ is K; B₄ is absent; B₅ is absent; all six (6) B₆ residues are L; B₇ is S; B₈ is L; B₉ is V; B₁₀ is L; and the string of three (3) B₁₁ residues is as follows: A-A-S.

### Variants of SEQ ID NO. 9 (Formula III)

In some embodiments, the pre-protein signal peptide comprises an amino acid sequence represented by:

C₁-(C₂)ᵣ-(C₃)ₜ-(C₄)ᵤ-[(C₅)ᵥ-(C₆)_{w}]ₓ-(C₇)_{y}-(C₈)_{z}-(C₉)-(C₁₀)-(C₁₁)-[(C₁₂)-(C₁₃)]ₐ (Formula III)

wherein C₂-C₁₃ have the properties described in Table 5 below:

TABLE 5

| | Isoelectric | Molecular | HP | |
|---|---|---|---|---|
| AA Label | point | Weight | Index | Helicity |
| C₂ | 5.6-10.8 | 105-175 | −5.1-0.6 | 0.8-1 |
| C₃, C₅, C₈, | 2.75-10.8 | 75-205 | −5.1-34 | 0.5-1.3 |
| and C₁₀ | | | | |
| C₄ and C₇ | 5-10.8 | 75-205 | −5.1-34 | 0.5-1.3 |
| C₆, C₉, C₁₁, | 2.75-9.8 | 75-205 | −4-34 | 0.5-1.3 |
| and C₁₂ | | | | |
| C₁₃ | 5.6-6.3 | 105-120 | 0-9.4 | 0.5-1.1 |

wherein r is an integer selected from 1-3;
t, u, y, and z are independently integers selected from 0-3 (inclusive);
each v and w are independently integers selected from 0-2 (inclusive);
x is an integer selected from 2-10 (inclusive); and
a is 0 or 1.

In some embodiments, C₁ is methionine. In some embodiments, each C₂ is, independently, an amino acid having an isoelectric point of about 5.6 to about 10.8, a molecular weight of about 105 g/mol to about 175 g/mol, a hydropathy index of about −5.1 to about 0.6, and a helicity of about 0.8 to about 1. In some embodiments, each C₃, C₅, C₈, and C₁₀ is each, independently, an amino acid having an isoelectric point of about 2.75 to about 10.8, a molecular weight of about 75 g/mol to about 205 g/mol, a hydropathy index of about −5.1 to about 34, and a helicity of about 0.5 to about 1.3. In some embodiments, each C₃ is, independently, an amino acid having an isoelectric point of about 2.75 to about 10.8, a molecular weight of about 75 g/mol to about 205 g/mol, a hydropathy index of about −5.1 to about 34, and a helicity of about 0.5 to about 1.3. In some embodiments, each C₅ is, independently, an amino acid having an isoelectric point of about 2.75 to about 10.8, a molecular weight of about 75 g/mol to about 205 g/mol, a hydropathy index of about −5.1 to about 34, and a helicity of about 0.5 to about 1.3. In some embodiments, each C₈ is, independently, an amino acid having an isoelectric point of about 2.75 to about 10.8, a molecular weight of about 75 g/mol to about 205 g/mol, a hydropathy index of about −5.1 to about 34, and a helicity of about 0.5 to about 1.3. In some embodiments, C₁₀ is an amino acid having an isoelectric point of about 2.75 to about 10.8, a molecular weight of about 75 g/mol to about 205 g/mol, a hydropathy index of about −5.1 to about 34, and a helicity of about 0.5 to about 1.3. In some embodiments, each C₄ and C₇ is each, independently, an amino acid having an isoelectric point of about 5 to about 10.8, a molecular weight of about 75 g/mol to about 205 g/mol, a hydropathy index of about −5.1 to about 34, and a helicity of about 0.5 to about 1.3. In some embodiments, each C₄ is, independently, an amino acid having an isoelectric point of about 5 to about 10.8, a molecular weight of about 75 g/mol to about 205 g/mol, a hydropathy index of about −5.1 to about 34, and a helicity of about 0.5 to about 1.3. In some embodiments, each C₇ is, independently, an amino acid having an isoelectric point of about 5 to about 10.8, a molecular weight of about 75 g/mol to about 205 g/mol, a hydropathy index of about −5.1 to about 34, and a helicity of about 0.5 to about 1.3. In some embodiments, each C₆, C₉, C₁₁, and C₁₂ is each, independently, an amino acid having an isoelectric point of about 2.75 to about 9.8, a molecular weight of about 75 g/mol to about 205 g/mol; a hydropathy index of about −4 to about 34, and a helicity of about 0.5 to about 1.3. In some embodiments, each C₆ is each, independently, an amino acid having an isoelectric point of about 2.75 to about 9.8, a molecular weight of about 75 g/mol to about 205 g/mol; a hydropathy index of about −4 to about 34, and a helicity of about 0.5 to about 1.3. In some embodiments, C₉ is an amino acid having an isoelectric point of about 2.75 to about 9.8, a molecular weight of about 75 g/mol to about 205 g/mol; a hydropathy index of about −4 to about 34, and a helicity of about 0.5 to about 1.3. In some embodiments, C₁₁ is an amino acid having an isoelectric point of about 2.75 to about 9.8, a molecular weight of about 75 g/mol to about 205 g/mol; a hydropathy index of about −4 to about 34, and a helicity of about 0.5 to about 1.3. In some embodiments, C₁₂ is an amino acid having an isoelectric point of about 2.75 to about 9.8, a molecular weight of about 75 g/mol to about 205 g/mol; a hydropathy index of about −4 to about 34, and a helicity of about 0.5 to about 1.3. In some embodiments, C₁₃ is an amino acid having an isoelectric point of about 5.6 to about 6.3, a molecular weight of about 105 g/mol to about 120 g/mol, a hydropathy index of about 0 to about 9.4, and a helicity of about 0.5 to about 1.1.

In some embodiments, r is 1. In some embodiments, r is 2, in some embodiments, r is 3. In some embodiments, t is 0. In some embodiments, t is 1. In some embodiments, t is 2. In some embodiments, t is 3. In some embodiments, u is 0. In some embodiments, u is 1. In some embodiments, u is 2. In some embodiments, u is 3. In some embodiments, y is 0. In some embodiments, y is 1. In some embodiments, y is 2. In some embodiments, y is 3. In some embodiments, z is 0. In some embodiments, z is 1. In some embodiments, z is 2. In some embodiments, z is 3. In some embodiments, v is 0. In some embodiments, v is 1. In some embodiments, v is 2. In some embodiments, w is 0. In some embodiments, w is 1. In some embodiments, w is 2. In some embodiments, x may be an integer selected from 3-9, 4-8, 6-10, 8-10, 2-5, and 3-6. In some embodiments, x is 2. In some embodiments, x is 3. In some embodiments, x is 4. In some embodiments, x is 5. In some embodiments, x is 6. In some embodiments, x is 7. In some embodiments, x is 8. In some embodiments, x is 9. In some embodiments, x is 10. In some embodiments a is 0 and the residues given by [(C₁₂)-(C₁₃)]ₐ are absent. In some embodiments, a is 1 and the residues given by [(C₁₂)-(C₁₃)]ₐ are present. It is to be understood that the values of r, t, u, y, z, v, w, and x are each independently selected, and the value of any variable r, t, u, y, z, v, w, or x is independent of the values selected for the other variables. In some embodiments, each C₃, C₅, C₈, and C₁₀ is each independently, an amino acid selected from the group consisting of L, F, I, V, A, W, Y, T, Q, S, H, C, N, D, R, P, K, G, E, and M. In some embodiments, each C₃, C₅, C₈, and C₁₀ is each, independently, an amino acid selected from the group consisting of L, F, I, V, and A. In some embodiments, each C₃ is, independently, an amino acid selected from the group consisting of L, F, I, V, A, W, Y, T, Q, S, H, C, N, D, R, P, K, G, E, and M. In some embodiments, each C₃ is, independently, an amino acid selected from the group consisting of L, F, I, V, and A. In some embodiments, each C₅ is, independently, an amino acid selected from the group consisting of L, F, I, V, A, W, Y, T, Q, S, H, C, N, D, R, P, K, G, E, and M. In some embodiments, each C₅ is, independently, an amino acid selected from the group consisting of L, F, I, V, and A. In some embodiments, each C₈ is, independently, an amino acid selected from the group consisting of L, F, I, V, A, W, Y, T, Q, S, H, C, N, D, R, P, K, G, E, and M. In some embodiments, each C₈ is, independently, an amino acid selected from the group consisting of L, F, I, V, and A. In some embodiments, C₁₀ is an amino acid selected from the group consisting of L, F, I, V, A, W, Y, T, Q, S, H, C, N, D, R, P, K, G, E, and M. In some embodiments, C₁₀ is an amino acid selected from the group consisting of L, F, I, V, and A. In some embodiments, each C₆, C₉, C₁₁, and C₁₂ is each, independently, an amino acid selected from the group consisting of A, S, V, G, I, L, F, C, T, K, P, Q, N, Y, E, D, M, and W. In some embodiments, each C₆, C₉, C₁₁, and C₁₂ is each, independently, an amino acid selected from the group consisting of A and S. In some embodiments, each C₆ is, independently, an amino acid selected from the group consisting of A, S, V, G, I, L, F, C, T, K, P, Q, N, Y, E, D, M, and W. In some embodiments, each C₆ is, independently, an amino acid selected from the group consisting of A and S. In some embodiments, C₉ is an amino acid selected from the group consisting of A, S, V, G, I, L, F, C, T, K, P, Q, N, Y, E, D, M, and W. In some embodiments, C₉ is an amino acid selected from the group consisting of A and S. In some embodiments, C₁₁ is an amino acid selected from the group consisting of A, S, V, G, I, L, F, C, T, K, P, Q, N, Y, E, D, M, and W. In some embodiments, C₁₁ is an amino acid selected from the group consisting of A and S. In some embodiments, C₁₂ is an amino acid selected from the group consisting of A, S, V, G, I, L, F, C, T, K, P, Q, N, Y, E, D, M, and W. In some embodiments, C₁₂ is an amino acid selected from the group consisting of A and S. In some embodiments, each C₂ is, independently, an amino acid selected from the group consisting of K, R, H, S, and Q. In some embodiments, C₁₃ is an amino acid selected from the group consisting of P, T, and S. In some embodiments, each C₄ and C₇ is each, independently, an amino acid selected from the group consisting of S, N, Q, R, T, K, A, Y, H, V, I, F, G, W, C, P, and L. In some embodiments, each C₄ and C₇ is each, independently, an amino acid selected from the group consisting of S, N, Q, R, T, K, A, and Y. In some embodiments, each C₄ is, independently, an amino acid selected from the group consisting of S, N, Q, R, T, K, A, Y, H, V, I, F, G, W, C, P, and L. In some embodiments, each C₄ is, independently, an amino acid selected from the group consisting of S, N, Q, R, T, K, A, and Y. In some embodiments, each C₇ is, independently, an amino acid selected from the group consisting of S, N, Q, R, T, K, A, Y, H, V, I, F, G, W, C, P, and L. In some embodiments, each C₇ is, independently, an amino acid selected from the group consisting of S, N, Q, R, T, K, A, and Y. In embodiments where any one of r, t, u, y, z, v, w, and x are an integer greater than 1, each amino acid in the group described by the r, t, u, y, z, v, w, and x are independently chosen from the disclosed group of amino acids and therefore may be the same or different, as described for herein.

Further, in consideration of [(C₅)ᵥ-(C₆)_{w}]ₓ, it is to be understood that in embodiments where x is an integer greater than 1, the formula [(C₅)ᵥ-(C₆)_{w}]ₓ does not indicate that [(C₅)ᵥ-(C₆)_{w}] is repeated x number of times. Rather, when (C₅)ᵥ-(C₆)_{w} is expanded due to an x greater than 1, each instance of C₅ can independently be selected from an appropriate amino acid as detailed above and likewise each instance of C₆ can independently be selected from an appropriate amino acid as detailed above. For example, in considering a hypothetical of [(C₅)₁-(C₆)₁]₂, the formula could produce the sequence L-A-L-A (SEQ ID NO: 101) wherein the first and second C₅ are both L and the first and second C₆ are both A, and could likewise produce L-A-V-C(SEQ ID NO: 102), wherein the first C₅ is L, the first C₆ is A, the second C₅ is V, and the second C₆ is C. In a further example, in considering a hypothetical of [(C₅)₁-(C₆)₁]₃, the formula could produce the sequence L-A-L-A-L-A (SEQ ID NO: 103) wherein the first, second, and third C₅ are all L and the first, second, and third C₆ are all A, and could likewise produce L-A-V-C-H-P (SEQ ID NO: 104), wherein the first C₅ is L, the first C₆ is A, the second C₅ is V, the second C₆ is C, the third C₅ is H, and the third C₆ is P. The same functionality of x holds true for the values of v and w. For example, in considering a hypothetical of [(C₅)ᵥ-(C₆)_{w}]₂, each instance of v and w may be an integer from 0 to 2 as described above. Thus, the first instance of v and the second instance of v may each be 1, or the first instance of v may be 1 and the second instance of v may be 2.

Thus, for example, when considering the formula of Formula III C₁-(C₂)ᵣ-(C₃)ₜ-(C₄)ᵤ-[(C₅)ᵥ-(C₆)_{w}]ₓ-(C₇)_{y}-(C₈)_{z}-(C₉)-(C₁₀)-(C₁₁)-[(C₁₂)-(C₁₃)]ₐ wherein x is 3, one can also envision the formula of Formula III to be written as:

C₁-(C₂)ᵣ-(C₃)ₜ-(C₄)ᵤ-(C₅)ᵥ-(C₆)_{w}-(C₅)ᵥ-(C₆)_{w}-(C₅)ᵥ-(C₆)_{w}-(C₇)_{y}-(C₈)_{z}-(C₉)—(C₁₀)-(C₁₁)-[(C₁₂)-(C₁₃)]ₐ

wherein each v and w are selected, independently, from 0, 1 or 2, and each C₅ and C₆ are selected, independently, from an appropriate amino acid as outlined above. This meaning, unless explicitly indicated otherwise, expands to all further formulas disclosed herein and below.

In some embodiments, the sequence of SEQ ID NO. 9 can be derived from Formula III as follows: r is 1, t is 2, u is 2, v is 2, w is 2, x is 2, y is 2, z is 1, and a is 1; C₁ is methionine, C₂ is K, the string of two (2) C₃ residues is as follows: L-S, the string of two (2) C₄ residues is as follows: S-L, the string of eight (8) residues given by [(C₅)₂-(C₆)₂]₂ is as follows: L-L-A-L-L-L-A-L (SEQ ID NO: 89), the string of two (2) C₇ residues is as follows: A-S, C₈ is L, C₉ is A, C₁₀ is L, C₁₁ is A, C₁₂ is present and is A, and C₁₃ is present and is P.

### Variants of SEQ ID NO. 12 (Formula IV)

In some embodiments, the pre-protein signal peptide comprises an amino acid sequence represented by:

D₁-(D₂)_{q}-(D₃)ᵣ-(D₄)ₜ-(D₅)ᵤ-[(D₆)ᵥ-(D₇)ₓ-(D₈)_{w}-(D₉)_{y}]_{z}-(D₁₀)-(D₁₁)-(D₁₂)-[(D₁₃)-(D₁₄)]ₐ (Formula IV)

wherein D₂-D₁₄ have the properties described in Table 6 below:

TABLE 6

| | Isoelectric | Molecular | HP | |
|---|---|---|---|---|
| AA Label | Point | Weight | Index | Helicity |
| D₂ | 2.7-10.8 | 75-205 | −5.1-34 | 0.5-1.3 |
| D₃ | 5-10.8 | 89-205 | −4-34 | 0.5-1.3 |
| D₄, D₉ and | 2.7-10.8 | 75-205 | −5.1-34 | 0.5-1.3 |
| D₁₁ | | | | |
| D₅ | 3.2-10.8 | 75-205 | −5.1-34 | 0.75-1.3 |
| D₈, D₁₀, | 2.7-10.8 | 75-182 | −5.1-32 | 0.75-1.3 |
| D₁₂, and | | | | |
| D₁₃ | | | | |
| D₇ | 5.4-6.1 | 117-205 | 2.5-34 | 1-1.3 |
| D₆ | 5-10.8 | 75-205 | −5.1-34 | 0.5-1.3 |
| D₁₄ | 2.7 -10.8 | 75-182 | −5.1-32 | 0.5-1.3 |

and q is an integer selected from 1, 2, or 3 (inclusive);
r, t and u are independently integers selected from 0, 1, 2, or 3 (inclusive);
each v, w, x, and y are independently integers selected from 0, 1, or 2 (inclusive);
z is an integer selected from 2, 3, 4, 5, 6, 7, 8, 9, or 10 (inclusive); and
a is 0 or 1.

In some embodiments, D₁ is methionine. In some embodiments, each D₂ is, independently, an amino acid having an isoelectric point of about 2.7 to about 10.8, a molecular weight of about 75 g/mol to about 205 g/mol, a hydropathy index of about −5.1 to about 34, and a helicity of about 0.5 to about 1.3. In some embodiments, each D₃ is, independently, an amino acid having an isoelectric point of about 5 to about 10.8, a molecular weight of about 89 g/mol to about 205 g/mol, a hydropathy index of about −4 to about 34, and a helicity of about 0.5 to about 1.3. In some embodiments, each D₄, D₉ and D₁₁ is each, independently, an amino acid having an isoelectric point of about 2.7 to about 10.8, a molecular weight of about 75 g/mol to about 205 g/mol, a hydropathy index of about −5.1 to about 34, and a helicity of about 0.5 to about 1.3. In some embodiments, each D₄ is, independently, an amino acid having an isoelectric point of about 2.7 to about 10.8, a molecular weight of about 75 g/mol to about 205 g/mol, a hydropathy index of about −5.1 to about 34, and a helicity of about 0.5 to about 1.3. In some embodiments, each D₉ is, independently, an amino acid having an isoelectric point of about 2.7 to about 10.8, a molecular weight of about 75 g/mol to about 205 g/mol, a hydropathy index of about −5.1 to about 34, and a helicity of about 0.5 to about 1.3. In some embodiments, D₁₁ is an amino acid having an isoelectric point of about 2.7 to about 10.8, a molecular weight of about 75 g/mol to about 205 g/mol, a hydropathy index of about −5.1 to about 34, and a helicity of about 0.5 to about 1.3. In some embodiments, each D₅ is, independently, an amino acid having an isoelectric point of about 3.2 to about 10.8, a molecular weight of about 75 g/mol to about 205 g/mol, a hydropathy index of about −5.1 to about 34, and a helicity of about 0.75 to about 1.3. In some embodiments, each D₆ is, independently, an amino acid having an isoelectric point from about 5 to about 10.8, a molecular weight of about 75 g/mol to about 205 g/mol, a hydropathy index of about −5.1 to about 34, and a helicity of about 0.5 to about 1.3. In some embodiments, each D₇ is, independently, an amino acid having an isoelectric point of about 5.4 to about 6.1, a molecular weight of about 117 g/mol to about 205 g/mol, a hydropathy index of about 2.5 to about 34, and a helicity of about 1 to about 1.3. In some embodiments, each D₈, D₁₀, D₁₂, and D₁₃ is each, independently, an amino acid having an isoelectric point of about 2.7 to about 10.8, a molecular weight of about 75 g/mol to about 182 g/mol, a hydropathy index of about −5.1 to about 32, and a helicity of about 0.75 to about 1.3. In some embodiments, each D₈ is, independently, an amino acid having an isoelectric point of about 2.7 to about 10.8, a molecular weight of about 75 g/mol to about 182 g/mol, a hydropathy index of about −5.1 to about 32, and a helicity of about 0.75 to about 1.3. In some embodiments, D₁₀ is an amino acid having an isoelectric point of about 2.7 to about 10.8, a molecular weight of about 75 g/mol to about 182 g/mol, a hydropathy index of about −5.1 to about 32, and a helicity of about 0.75 to about 1.3. In some embodiments, D₁₂ is an amino acid having an isoelectric point of about 2.7 to about 10.8, a molecular weight of about 75 g/mol to about 182 g/mol, a hydropathy index of about −5.1 to about 32, and a helicity of about 0.75 to about 1.3. In some embodiments, D₁₃ is an amino acid having an isoelectric point of about 2.7 to about 10.8, a molecular weight of about 75 g/mol to about 182 g/mol, a hydropathy index of about −5.1 to about 32, and a helicity of about 0.75 to about 1.3. In some embodiments, D₁₄ is an amino acid with an isoelectric point of about 2.7 to about 10.8, a molecular weight of about 75 g/mol to about 182 g/mol, a hydropathy index of about −5.1 to about 32, and a helicity of about 0.5 to about 1.3.

In some embodiments, q is 1. In some embodiments, q is 2. In some embodiments, q is 3. In some embodiments, r is 0. In some embodiments, r is 1. In some embodiments, r is 2. In some embodiments, r is 3. In some embodiments, t is 0. In some embodiments, t is 1. In some embodiments, t is 2. In some embodiments, t is 3. In some embodiments, u is 0. In some embodiments, u is 1. In some embodiments, u is 2. In some embodiments, u is 3. In some embodiments, v is 0. In some embodiments, v is 1. In some embodiments, v is 2. In some embodiments, w is 0. In some embodiments, w is 1. In some embodiments, w is 2. In some embodiments, x is 0. In some embodiments, x is 1. In some embodiments, x is 2. In some embodiments, y is 0. In some embodiments, y is 1. In some embodiments, y is 2. In some embodiments, z may be an integer selected from 3-9, 4-8, 6-10, 8-10, 2-5, or 3-6 (all inclusive). In some embodiments, z is 2. In some embodiments, z is 3. In some embodiments, z is 4. In some embodiments, z is 5. In some embodiments, z is 6. In some embodiments, z is 7. In some embodiments, z is 8. In some embodiments, z is 9. In some embodiments, z is 10. In some embodiments a is 0 and the residues given by [(D₁₃)-(D₁₄)]ₐ are absent. In some embodiments, a is 1 and the residues given by [(D₁₃)-(D₁₄)]ₐ are present. It is to be understood that the values of r, t, u, v, w, x, y, and z are each independently selected, and the value of any variable r, t, u, v, w, x, y, or z is independent of the values selected for the other variables. In some embodiments, each D₂ is, independently, an amino acid selected from the group consisting of K and R. In some embodiments, each D₃ is, independently, an amino acid selected from the group consisting of F, L, I, W, V, M, Y, P, C, A, Q, and S. In some embodiments, each D₄, D₉ and D₁₁ is each, independently, an amino acid selected from the group consisting of L, I, F, W, V, M, Y, A, T, N, S, G, E, D, C, Q, R, H, P, and K. In some embodiments, each D₄, D₉ and D₁₁ is each, independently, an amino acid selected from the group consisting of L and I. In some embodiments, each D₄ is, independently, an amino acid selected from the group consisting of L, I, F, W, V, M, Y, A, T, N, S, G, E, D, C, Q, R, H, P, and K. In some embodiments, each D₄ is, independently, an amino acid selected from the group consisting of L or I. In some embodiments, each D₉ is, independently, an amino acid selected from the group consisting of L, I, F, W, V, M, Y, A, T, N, S, G, E, D, C, Q, R, H, P, and K. In some embodiments, each D₉ is, independently, an amino acid selected from the group consisting of L and I. In some embodiments, D₉ is an amino acid selected from the group consisting of L, I, F, W, V, M, Y, A, T, N, S, G, E, D, C, Q, R, H, P, and K. In some embodiments, D₉ is an amino acid selected from the group consisting of L and I. In some embodiments, D₁₁ is an amino acid selected from the group consisting of L, I, F, W, V, M, Y, A, T, N, S, G, E, D, C, Q, R, H, P, and K. In some embodiments, D₁₁ is an amino acid selected from the group consisting of L and I. In some embodiments, each D₅ is, independently, an amino acid selected from the group consisting of S, N, Q, R, T, G, K, E, H, A, C, Y, V, W, I, F, and L. In some embodiments, each D₈, D₁₀, D₁₂, and D₁₃ is each, independently, an amino acid selected from the group consisting of A, S, T, G, V, L, C, Y, K, I, F, Q, N, H, R, E, D, and M. In some embodiments, each D₈, D₁₀, D₁₂, and D₁₃ is each, independently, an amino acid selected from the group consisting of A and S. In some embodiments, each D₈ is, independently, an amino acid selected from the group consisting of A, S, T, G, V, L, C, Y, K, I, F, Q, N, H, R, E, D, and M. In some embodiments, each D₈ is, independently, an amino acid selected from the group consisting of A and S. In some embodiments, D₁₀ is an amino acid selected from the group consisting of A, S, T, G, V, L, C, Y, K, I, F, Q, N, H, R, E, D, and M. In some embodiments, D₁₀ is an amino acid selected from the group consisting of A and S. In some embodiments, D₁₂ is an amino acid selected from the group consisting of A, S, T, G, V, L, C, Y, K, I, F, Q, N, H, R, E, D, and M. In some embodiments, D₁₂ is an amino acid selected from the group consisting of A and S. In some embodiments, D₁₃ is an amino acid selected from the group consisting of A, S, T, G, V, L, C, Y, K, I, F, Q, N, H, R, E, D, and M. In some embodiments, D₁₃ is an amino acid selected from the group consisting of A and S. In some embodiments, each D₇ is, independently, an amino acid selected from the group consisting of V, W, I, L, F, and T. In some embodiments, each D₆ is, independently, an amino acid selected from the group consisting of L, I, A, T, S, G, N, R K, Y, Q, C, H, W, and M. In some embodiments, each D₆ is, independently, an amino acid selected from the group consisting of L and I. In some embodiments, D₁₄ is an amino acid selected from the group consisting of P, Y, M, V, A, T, Q, S, N, G, I, E, D, L, F, R, K, and H. In embodiments where any one of r, t, u, v, w, x, y, and z are an integer greater than 1, each amino acid in the group described by the r, t, u, v, w, x, y, and z are independently chosen from the disclosed group of amino acids and therefore may be the same or different, as described for herein.

As outlined pertaining to Formula III, the portion of Formula IV given by -[(D₆)ᵥ-(D₇)ₓ-(D₈)_{w}-(D₉)_{y}]_{z} is not to be interpreted as “z” repeats of [(D₆)ᵥ-(D₇)ₓ-(D₈)_{w}-(D₉)_{y}], but rather, when expanded “z” times, each v, x, w, and y may be independently selected from an integer as provided for above, and each D₆, D₇, D₈, and D₉ may be independently selected from an appropriate amino acid as provided for above.

Thus, for example, when considering the formula of Formula IV D₁-(D₂)_{q}-(D₃)ᵣ-(D₄)ₜ-(D₅)ᵤ-[(D₆)ᵥ-(D₇)ₓ-(D₈)_{w}(D₉)_{y}]_{z}-(D₁₀)-(D₁₁)-(D₁₂)-[(D₁₃)-(D₁₄)]ₐ wherein z is 3, one can also envision the formula of Formula IV to be written as: D₁-(D₂)_{q}-(D₃)ᵣ-(D₄)ₜ-(D₅)ᵤ-(D₆)ᵥ-(D₇)ₓ-(D₈)_{w}-(D₉)_{y}-(D₆)ᵥ-(D₇)ₓ-(D₈)_{w}-(D₉)_{y}-(D₆)ᵥ-(D₇)ₓ-(D₈)_{w}-(D₉)_{y}-(D₁₀)-(D₁₁)-(D₁₂)-[(D₁₃)-(D₁₄)]ₐ wherein each v, x, w, and y are selected, independently, from 0, 1 or 2, and each D₆, D₇, D₈, and D₉ are selected, independently, from an appropriate amino acid as outlined above.

In some embodiments, the sequence of SEQ ID NO. 12 can be derived from Formula IV as follows: q is 1, r is 1, t is 1, u is 2, for every instance of z v is 0, for every instance of z x is 0, w is 1, y is 1, z is 6, and a is 1; D₁ is methionine; D₂ is K; D₃ is F; D₄ is L; the string of two (2) D₅ residues is as follows: S-L; for every instance of z D₆ is absent; for every instance of z D₇ is absent; the string of twelve (12) residues given by [(D₈)₁-(D₉)₁]₆ is as follows: L-L-A-L-V-A-A-L-A-L-A-L (SEQ ID NO: 90); D₁₀ is A; D₁₁ is L; D₁₂ is A; D₁₃ is present and is A; and D₁₄ is present and is P.

### Variants of SEQ ID NOs. 14, 15, and 16 (Formula V)

In some embodiments, the pre-protein signal peptide comprises an amino acid sequence represented by:

E₁-[(E₂)ᵢ(E₃)ⱼ-(E₄)_{q}]ᵣ-(E₅)ₜ-(E₆)ᵤ-(E₇)ᵥ-[(E₈)_{w}-(E₉)ₓ]_{y}-(E₁₀)_{z}-(E₁₁)-(E₁₂)-(E₁₃)-[(E₁₄)-(E₁₅)]ₐ Formula V

wherein E₂-E₁₅ have the properties described in Table 7 below:

TABLE 7

| | Isoelectric | Molecular | HP | |
|---|---|---|---|---|
| AA Label | Point | Weight | Index | Helicity |
| E₂ | 3.2-10.8 | 105-175 | −4-1 | 0.85-1 |
| E₃ | 2.7-10.8 | 75-205 | −5.1-33.5 | 0.57-1.3 |
| E₄ | 5-10.8 | 105-205 | −5.1-33.5 | 0.57-1.3 |
| E₅ and E₈ | 2.7-10.8 | 75-205 | −5.1-33.5 | 0.57-1.3 |
| E₆ | 5-10.8 | 89-205 | −5.1-33.5 | 0.57-1.3 |
| E₇ | 5-9.75 | 75-205 | −4-33.5 | 0.79-1.3 |
| E₉, E₁₃, | 2.7-10.8 | 75-205 | −5.1-33.5 | 0.57-1.3 |
| and E₁₄ | | | | |
| E₁₀ and E₁₂ | 5-10.8 | 75-205 | −5.1-34 | 0.5-1.3 |
| E₁₁ | 5-9.75 | 89-205 | −4-33.5 | 0.79-1.3 |
| E₁₅ | 2.7-10.8 | 75-205 | −4-15.5 | 0.57-1.2 |

wherein each i, j, q, w, x, and a are independently 0 or 1;
r is an integer selected from 1, 2, or 3 (inclusive);
t, u, v, and z are independently integers selected from 0, 1, 2, or 3 (inclusive); and
y is an integer selected from 2, 3, 4, 5, 6, 7, 8, 9, or 10 (inclusive).

In some embodiments, E₁ is methionine. In some embodiments, each E₂ is, independently, an amino acid having an isoelectric point of about 3.2 to about 10.8, a molecular weight of about 105 g/mol to about 175 g/mol, a hydropathy index of about −4 to about 1, and a helicity of about 0.85 to about 1. In some embodiments, each E₃ is, independently, an amino acid having an isoelectric point of about 2.7 to about 10.8, a molecular weight of about 75.1 g/mol to about 205 g/mol, a hydropathy index of about −5.1 to about 33.5, and a helicity of about 0.57 to about 1.3. In some embodiments, each E₄ is, independently, an amino acid having an isoelectric point of about 5 to about 10.8, a molecular weight of about 105 g/mol to about 205 g/mol, a hydropathy index of about −5.1 to about 33.5, and a helicity of about 0.57 to about 1.3. In some embodiments, each E₅ and E₈ is each, independently, an amino acid having an isoelectric point of about 2.7 to about 10.8, a molecular weight of about 75 g/mol to about 205 g/mol, a hydropathy index of about −5.1 to about 33.5, and a helicity of about 0.57 to about 1.3. In some embodiments, each E₅ is, independently, an amino acid having an isoelectric point of about 2.7 to about 10.8, a molecular weight of about 75 g/mol to about 205 g/mol, a hydropathy index of about −5.1 to about 33.5, and a helicity of about 0.57 to about 1.3. In some embodiments, each E₈ is, independently, an amino acid having an isoelectric point of about 2.7 to about 10.8, a molecular weight of about 75 g/mol to about 205 g/mol, a hydropathy index of about −5.1 to about 33.5, and a helicity of about 0.57 to about 1.3. In some embodiments, each E₆ is, independently, an amino acid having an isoelectric point of about 5 to about 10.8, a molecular weight of about 89 g/mol to about 205 g/mol, a hydropathy index of about −5.1 to about 33.5, and a helicity of about 0.57 to about 1.3. In some embodiments, each E₇ is, independently, an amino acid having an isoelectric point of about 5 to about 9.75, a molecular weight of about 75 g/mol to about 205 g/mol, a hydropathy index of about −4 to about 33.5, and a helicity of about 0.79 to about 1.3. In some embodiments, each E₉, E₁₃, and E₁₄ is each, independently, an amino acid having an isoelectric point of about 2.7 to about 10.8, a molecular weight of about 75 g/mol to about 205 g/mol, a hydropathy index of about −5.1 to about 33.5, and a helicity of about 0.57 to about 1.3. In some embodiments, each E₉ is, independently, an amino acid having an isoelectric point of about 2.7 to about 10.8, a molecular weight of about 75 g/mol to about 205 g/mol, a hydropathy index of about −5.1 to about 33.5, and a helicity of about 0.57 to about 1.3. In some embodiments, E₁₃ is an amino acid having an isoelectric point of about 2.7 to about 10.8, a molecular weight of about 75 g/mol to about 205 g/mol, a hydropathy index of about −5.1 to about 33.5, and a helicity of about 0.57 to about 1.3. In some embodiments, E₁₄ is an amino acid having an isoelectric point of about 2.7 to about 10.8, a molecular weight of about 75 g/mol to about 205 g/mol, a hydropathy index of about −5.1 to about 33.5, and a helicity of about 0.57 to about 1.3. In some embodiments, each E₁₀ and E₁₂ is, independently, an amino acid having an isoelectric point of about 5 to about 10.8, a molecular weight of about 75 g/mol to about 205 g/mol, a hydropathy index of about −5.1 to about 34, and a helicity of about 0.5 to about 1.3. In some embodiments, each E₁₀ is, independently, an amino acid having an isoelectric point of about 5 to about 10.8, a molecular weight of about 75 g/mol to about 205 g/mol, a hydropathy index of about −5.1 to about 34, and a helicity of about 0.5 to about 1.3. In some embodiments, E₁₂ is an amino acid having an isoelectric point of about 5 to about 10.8, a molecular weight of about 75 g/mol to about 205 g/mol, a hydropathy index of about −5.1 to about 34, and a helicity of about 0.5 to about 1.3. In some embodiments, E₁₁ is an amino acid having an isoelectric point of about 5 to about 9.75, a molecular weight of about 89 g/mol to about 205 g/mol, a hydropathy index of about −4 to about 33.5, and a helicity of about 0.79 to about 1.3. In some embodiments, E₁₅ is an amino acid having an isoelectric point of about 2.7 to about 10.8, a molecular weight of about 75 g/mol to about 205 g/mol, a hydropathy index of about −4 to about 15.5, and a helicity of about 0.57 to about 1.2.

In some embodiments, i is 0. In some embodiments, i is 1. In some embodiments, j is 0. In some embodiments, j is 1. In some embodiments, q is 0. In some embodiments, q is 1. In some embodiments, w is 0. In some embodiments, w is 1. In some embodiments, x is 0. In some embodiments, x is 1. In some embodiments, r is 1. In some embodiments, r is 2. In some embodiments, r is 3. In some embodiments, t is 0. In some embodiments, t is 1. In some embodiments, t is 2. In some embodiments, t is 3. In some embodiments, u is 0. In some embodiments, u is 1. In some embodiments, u is 2. In some embodiments, u is 3. In some embodiments, v is 0. In some embodiments, v is 1. In some embodiments, v is 2. In some embodiments, v is 3. In some embodiments, z is 0. In some embodiments, z is 1. In some embodiments, z is 2. In some embodiments, z is 3. In some embodiments, y may be an integer selected from 3-9, 4-8, 6-10, 8-10, 2-5, or 3-6 (all inclusive). In some embodiments, y is 2. In some embodiments, y is 3. In some embodiments, y is 4. In some embodiments, y is 5. In some embodiments, y is 6. In some embodiments, y is 7. In some embodiments, y is 8. In some embodiments, y is 9. In some embodiments, y is 10. In some embodiments a is 0 and the residues given by [(E₁₄)-(E₁₅)]ₐ are absent. In some embodiments, a is 1 and the residues given by [(E₁₄)-(E₁₅)]ₐ are present. It is to be understood that the values of i, j, q, w, x, r, t, u, v, z, and y are each independently selected, and the value of any variable i, j, q, w, x, r, t, u, v, z, or y is independent of the values selected for the other variables. In some embodiments, each E₂ is, independently, an amino acid selected from the group consisting of K, R, S, Q, and E. In some embodiments, each E₃ is, independently, an amino acid selected from the group consisting of F, L, I, W, V, Y, P, A, T, Q, N, S, G, D, R, K, and H. In some embodiments, each E₃ is, independently, an amino acid selected from the group consisting of F, L, I, W, V, and Y. In some embodiments, each E₄ is, independently, an amino acid selected from the group consisting of K, R, H, S, C, P, Y, M, V, W, I, L, and F. In some embodiments, each E₄ may independently be K, R, H, and S. In some embodiments, each E₅ and E₈ is each, independently, an amino acid selected from the group consisting of L, I, F, V, C, A, Y, T, Q, N, S, K, H, W, G, D, M, P, E, and R. In some embodiments, each E₅ and E₈ is each, independently, an amino acid selected from the group consisting of L, I, F, V, and C. In some embodiments, each E₅ is, independently, an amino acid selected from the group consisting of L, I, F, V, C, A, Y, T, Q, N, S, K, H, W, G, D, M, P, E, and R. In some embodiments, each E₅ is, independently, an amino acid selected from the group consisting of L, I, F, V, and C. In some embodiments, each E₈ is, independently, an amino acid selected from the group consisting of L, I, F, V, C, A, Y, T, Q, N, S, K, H, W, G, D, M, P, E, and R. In some embodiments, each E₈ is, independently, an amino acid selected from the group consisting of L, I, F, V, and C. In some embodiments, each E₆ is, independently, an amino acid selected from the group consisting of T, Q, S, A, C, R, K, H, P, V, W, I, F, and L. In some embodiments, each E₇ is, independently, an amino acid selected from the group consisting of S, G, K, A, C, Y, V, and W. In some embodiments, each E₉, E₁₃, and E₁₄ is each, independently, an amino acid selected from the group consisting of A, T, G, S, V, I, L, Y, W, F, C, Q, N, P, E, M, R, K, D, and H. In some embodiments, each E₉, E₁₃, and E₁₄ is each, independently, an amino acid selected from the group consisting of A, T, G, S, V, I, and L. In some embodiments, each E₉ is, independently, an amino acid selected from the group consisting of A, T, G, S, V, I, L, Y, W, F, C, Q, N, P, E, M, R, K, D, and H. In some embodiments, each E₉ is, independently, an amino acid selected from the group consisting of A, T, G, S, V, I, and L. In some embodiments, each E₁₀ and E₁₂ is, independently, an amino acid selected from the group consisting of L, F, I, V, C, Y, T, Q, N, S, K, H, M, G, A, W, D, P, E, and R. In some embodiments, each E₁₀ and E₁₂ is, independently, an amino acid selected from the group consisting of L, F, I, V, and C. In some embodiments, each E₁₀ is, independently, an amino acid selected from the group consisting of L, F, I, V, C, Y, T, Q, N, S, K, H, M, G, A, W, D, P, E, and R. In some embodiments, each E₁₀ is, independently, an amino acid selected from the group consisting of L, F, I, V, and C. In some embodiments, E₁₂ is an amino acid selected from the group consisting of L, F, I, V, C, Y, T, Q, N, S, K, H, M, G, A, W, D, P, E, and R. In some embodiments, E₁₂ is an amino acid selected from the group consisting of L, F, I, V, and C. In some embodiments, E₁₃ is an amino acid selected from the group consisting of A, T, G, S, V, I, L, Y, W, F, C, Q, N, P, E, M, R, K, D, and H. In some embodiments, E₁₃ is an amino acid selected from the group consisting of A, T, G, S, V, I, and L. In some embodiments, E₁₄ is an amino acid selected from the group consisting of A, T, G, S, V, I, L, Y, W, F, C, Q, N, P, E, M, R, K, D, and H. In some embodiments, E₁₄ is an amino acid selected from the group consisting of A, T, G, S, V, I, and L. In some embodiments, each E₁₁ is, independently, an amino acid selected from the group consisting of V, W, I, C, L, A, T, S, and K. In some embodiments, each E₁₅ is, independently, an amino acid selected from the group consisting of S, N, R, T, G, K, E, D, P, and Y. In embodiments where any one of r, t, u, v, z, and y are an integer greater than 1, each amino acid in the group described by the r, t, u, v, z, and y are independently chosen from the disclosed group of amino acids and therefore may be the same or different, as described for herein.

As outlined pertaining to Formula III, the portion of Formula V given by [(E₈)_{w}-(E₉)ₓ]_{y} is not to be interpreted as “y” repeats of [(E₈)_{w}-(E₉)ₓ], but rather, when expanded “y” times, each w and x may be independently selected from an integer as provided for above, and each E₈ and E₉ may be independently selected from an appropriate amino acid as provided for above. The same is to be understood for the portion of Formula V given by [(E₂)ᵢ-(E₃)ⱼ-(E₄)_{q}]ᵣ.

Thus, for example, when considering the formula of Formula V, E₁-[(E₂)ᵢ-(E₃)ⱼ-(E₄)_{q}]ᵣ-(E₅)ₜ-(E₆)ᵤ-(E₇)ᵥ-[(E₈)_{w}(E₉)ₓ]_{y}-(E₁₀)_{z}-(E₁₁)-(E₁₂)-(E₁₃)-[(E₁₄)-(E₁₅)]ₐ, wherein r is 2 and y is 2, one can also envision the formula of Formula V to be written as: E₁-(E₂)ᵢ-(E₃)ⱼ-(E₄)_{q}-(E₂)ᵢ-(E₃)ⱼ-(E₄)_{q}-(E₅)ₜ-(E₆)ᵤ-(E₇)ᵥ-(E₈)_{w}-(E₉)ₓ-(E₈)_{w}(E₉)ₓ-(E₁₀)_{z}-(E₁₁)-(E₁₂)-(E₁₃)-[(E₁₄)-(E₁₅)]ₐ wherein each i, j, q, w and x are selected, independently, from 0 or 1, and each E₂, E₃, E₄, E₅, and E₉ are selected, independently, from an appropriate amino acid as outlined above.

In some embodiments, the sequence of SEQ ID NO. 14 can be derived from Formula V as follows: i is 1, j is 1, q is 1, r is 1, t is 1, u is 2, v is 0, w is 1, x is 1, y is 5, z is 0, and a is 1; E₁ is methionine; E₂ is K; E₃ is F; E₄ is K; E₅ is L; the string of two (2) E₆ residues is as follows: T-L; E₇ is absent; the string of ten (10) residues given by [(E₈)₁-(E₉)₁]₅ is as follows: L-A-A-L-L-A-L-A-A-L (SEQ ID NO: 91); E₁₀ is absent; E₁₁ is V; E₁₂ is L; E₁₃ is A; E₁₄ is present and is A; and E₁₅ is present and is S.

In some embodiments, the sequence of SEQ ID NO. 15 can be derived from Formula V as follows: i is 1, j is 1, q is 1, r is 1, t is 1, u is 2, v is 0, w is 1, x is 1, y is 4, z is 0, and a is 1; E₁ is methionine; E₂ is K; E₃ is L; E₄ is S; E₅ is S; the string of two (2) E₆ residues is as follows: I-L; E₇ is absent; the string of eight (8) residues given by [(E₈)₁-(E₉)₁]₄ is as follows: L-L-L-A-L-L-A-L (SEQ ID NO: 92); E₁₀ is absent; En is V; E₁₂ is L; E₁₃ is A; E₁₄ is present and is A; and E₁₅ is present and is S.

In some embodiments, the sequence of SEQ ID NO. 16 can be derived from Formula V as follows: i is 1, j is 1, q is 1, r is 2, t is 1, u is 2, v is 0, w is 1, x is 1, y is 3, z is 0, and a is 1; E₁ is methionine; the string of six (6) residues given by [(E₂)₁-(E₃)₁-(E₄)₁]₂ is as follows: K-L-L-S-L-L (SEQ ID NO: 106); E₅ is A; the string of two (2) E₆ residues is as follows: L-L; E₇ is absent; the string of six (6) residues given by [(E₈)₁-(E₉)₁]₃ is as follows: L-L-L-A-S-L (SEQ ID NO: 93); E₁₀ is absent; E₁₁ is V; E₁₂ is L; E₁₃ is A; E₁₄ is present and is A; and E₁₅ is present and is S.

### Variants of SEQ ID NOs. 31, 32, and 33 (Formula IX)

In some embodiments, the pre-protein signal peptide comprises an amino acid sequence represented by:

F₁-(F₂)ᵥ-(F₃)_{w}-[(F₄)ₓ-(F₅)_{y}]_{z}-(F₆)-(F₇)-(F₈)-[(F₉)-(F₁₀)]ₐ (Formula IX)

wherein F₁-F₁₀ have the properties described in Table 8 below:

TABLE 8

| | Isoelectric | Molecular | HP | Helicity |
|---|---|---|---|---|
| Label | Point | Weight | Index | Min |
| F₁ | 5.4-11 | 89-175 | −4-31 | 0.9-1.3 |
| F₂ | 3-11 | 75-205 | −5.1-34 | 0.5-1.3 |
| F₃ and F₇ | 3-11 | 75-205 | −5.1-34 | 0.5-1.3 |
| F₄ | 3-11 | 75-205 | −5.1-34 | 0.5-1.3 |
| F₅, F₆, F₈, | 3-11 | 75-205 | −5.1-34 | 0.5-1.3 |
| and F₉ | | | | |
| F₁₀ | 3-11 | 75-205 | −5.1-34 | 0.5-1.3 |

wherein v and w are independently integers selected from 0, 1, 2, or 3 (inclusive); and
x and y are independently selected from 0, 1, 2, 3, or 4;
z is an integer selected from 1, 2, 3, 4, 5, 6, 7, or 8 (inclusive); and
a is 0 or 1.

In some embodiments, F₁ is an amino acid having an isoelectric point of about 5.4 to about 11, a molecular weight of about 89 g/mol to about 175 g/mol; a hydropathy index of about −4 to about 31, and a helicity of about 0.9 to about 1.3. In some embodiments, each F₂ is, independently, an amino acid having an isoelectric point of about 3 to about 11, a molecular weight of about 89 g/mol to about 205 g/mol; a hydropathy index of about −5.1 to about 34, and a helicity of about 0.5 to about 1.3. In some embodiments, each F₃ and F₇ is each, independently, an amino acid having an isoelectric point of about 3 to about 11, a molecular weight of about 89 g/mol to about 205 g/mol; a hydropathy index of about −5.1 to about 34, and a helicity of about 0.5 to about 1.3. In some embodiments, each F₃ is, independently, an amino acid having an isoelectric point of about 3 to about 11, a molecular weight of about 89 g/mol to about 205 g/mol; a hydropathy index of about −5.1 to about 34, and a helicity of about 0.5 to about 1.3. In some embodiments, F₇ is an amino acid having an isoelectric point of about 3 to about 11, a molecular weight of about 89 g/mol to about 205 g/mol; a hydropathy index of about −5.1 to about 34, and a helicity of about 0.5 to about 1.3. In some embodiments, each F₄ is, independently, an amino acid having an isoelectric point of about 3 to about 11, a molecular weight of about 89 g/mol to about 205 g/mol; a hydropathy index of about −5.1 to about 34, and a helicity of about 0.5 to about 1.3. In some embodiments, each F₅, F₆, F₈, and F₉ is each, independently, an amino acid having an isoelectric point of about 3 to about 11, a molecular weight of about 89 g/mol to about 205 g/mol; a hydropathy index of about −5.1 to about 34, and a helicity of about 0.5 to about 1.3. In some embodiments, each F₅ is, independently, an amino acid having an isoelectric point of about 3 to about 11, a molecular weight of about 89 g/mol to about 205 g/mol; a hydropathy index of about −5.1 to about 34, and a helicity of about 0.5 to about 1.3. In some embodiments, F₆ is an amino acid having an isoelectric point of about 3 to about 11, a molecular weight of about 89 g/mol to about 205 g/mol; a hydropathy index of about −5.1 to about 34, and a helicity of about 0.5 to about 1.3. In some embodiments, F₈ is an amino acid having an isoelectric point of about 3 to about 11, a molecular weight of about 89 g/mol to about 205 g/mol; a hydropathy index of about −5.1 to about 34, and a helicity of about 0.5 to about 1.3. In some embodiments, F₉ is an amino acid having an isoelectric point of about 3 to about 11, a molecular weight of about 89 g/mol to about 205 g/mol; a hydropathy index of about −5.1 to about 34, and a helicity of about 0.5 to about 1.3. In some embodiments, F₁₀ is an amino acid having an isoelectric point of about 3 to about 11, a molecular weight of about 89 g/mol to about 205 g/mol; a hydropathy index of about −5.1 to about 34, and a helicity of about 0.5 to about 1.3.

In some embodiments, v is 0. In some embodiments, v is 1. In some embodiments, v is 2. In some embodiments, v is 3. In some embodiments, w is 0. In some embodiments, w is 1. In some embodiments, w is 2. In some embodiments, w is 3. In some embodiments, x is 0. In some embodiments, x is 1. In some embodiments, x is 2. In some embodiments, x is 3. In some embodiments, x is 4. In some embodiments, y is 0. In some embodiments, y is 1. In some embodiments, y is 2. In some embodiments, y is 3. In some embodiments, y is 4. In some embodiments, z may be an integer selected from 3-8, 4-8, 6-8, 2-5, or 3-6 (all inclusive). In some embodiments, z is 1. In some embodiments, z is 2. In some embodiments, z is 3. In some embodiments, z is 4. In some embodiments, z is 5. In some embodiments, z is 6. In some embodiments, z is 7. In some embodiments, z is 8. In some embodiments a is 0 and the residues given by [(F₉)-(F₁₀)]ₐ are absent. In some embodiments, a is 1 and the residues given by [(F₉)-(F₁₀)]ₐ are present. It is to be understood that the values of v, w, x, y, and z are each independently selected, and the value of any variable v, w, x, y, or z is independent of the values selected for the other variables. In some embodiments, F₁ is an amino acid selected from the group consisting of M, F, L, A, S, or R. In some embodiments, each F₂ is, independently, an amino acid selected from the group consisting of K, R, H, S, G, N, Q, E, T, A, C, P, Y, V, W, I, L, and F. In some embodiments, each F₂ is, independently, an amino acid selected from the group consisting of K, R, H, S, G, N, Q, E, T, and A. In some embodiments, each F₃ and F₇ is, independently, an amino acid selected from the group consisting of S, Q, R, T, K, H, I, F, L, P, N, G, E, D, A, Y, M, V, W, and C. In some embodiments, each F₃ and F₇ is, independently an amino acid selected from the group consisting of S, Q, R, T, K, H, I, F, and L. In some embodiments, each F₄ is, independently, an amino acid selected from the group consisting of L, I, V, M, A, F, W, Y, P, C, T, Q, N, S, G, E, R, K, and H. In some embodiments, each F₄ is, independently, an amino acid selected from the group consisting of L, I, V, M, and A. In some embodiments, each F₅, F₆, F₈, and F₉ is each, independently, an amino acid selected from the group consisting of A, C, G, S, V, L, T, F, Q, N, P, Y, E, K, H, W, I, M, and R. In some embodiments, each F₅, F₆, F₈, and F₉ is each, independently, an amino acid selected from the group consisting of A, C, G, S, V, and L. In some embodiments, F₁₀ is an amino acid selected from the group consisting of P, C, Y, M, V, A, T, Q, S, N, W, G, I, E, L, F, R, K, and H. In embodiments where any one of v, w, x, y, and z are an integer greater than 1, each amino acid in the group described by the v, w, x, y, and z are independently chosen from the disclosed group of amino acids and therefore may be the same or different, as described for herein.

As outlined pertaining to Formula III, the portion of Formula IX given by [(F₄)ₓ-(F₅)_{y}]_{z} is not to be interpreted as “z” repeats of [(F₄)ₓ-(F₅)_{y}], but rather, when expanded “z” times, each x and y may be independently selected from an integer as provided for above, and each F₄ and F₅ may be independently selected from an appropriate amino acid as provided for above.

Thus, for example, when considering the formula of Formula IX F₁-(F₂)ᵥ-(F₃)_{w}-[(F₄)ₓ-(F₅)_{y}]_{z}-(F₆)—(F₇)-(F₈)-[(F₉)—(F₁₀)]ₐ wherein z is 3, one can also envision the formula of Formula IX to be written as: F₁-(F₂)ᵥ-(F₃)_{w}-(F₄)ₓ-(F₅)_{y}-(F₄)ₓ-(F₅)_{y}-(F₄)ₓ-(F₅)_{y}-(F₆)-(F₇)-(F₅)-[(F₉)-(F₁₀)]ₐ wherein each x and y are selected, independently, from 0, 1, 2, 3, or 4, and each F₄ and F₅ are selected, independently, from an appropriate amino acid as outlined above.

In some embodiments, the sequence of SEQ ID NO. 31 can be derived from Formula IX as follows: v is 3, w is 0, x is 1, y is 1, z is 6, and a is 1; F₁ is methionine; the string of three (3) F₂ residues is as follows: K-S-S; F₃ is absent; the string of twelve (12) residues given by [(F₄)₁-(F₅)₁]₆ is as follows: L-L-L-L-A-L-L-A-L-A-A-L (SEQ ID NO: 94); F₆ is A; F₇ is S; F₈ is A; F₉ is present and is A; and F₁₀ is present and is P.

In some embodiments, the sequence of SEQ ID NO. 32 can be derived from Formula IX as follows: v is 2, w is 0, x is 1, y is 1, z is 6, and a is 1; F₁ is methionine; the string of two (2) F₂ residues is as follows: K-S; F₃ is absent; the string of twelve (12) residues given by [(F₄)₁-(F₅)₁]₆ is as follows: S-L-L-L-L-L-L-A-L-A-S-L (SEQ ID NO: 95); F₆ is A; F₇ is L; F₈ is A; F₉ is present and is A; and F₁₀ is present and is P.

In some embodiments, the sequence of SEQ ID NO. 33 can be derived from Formula IX as follows: v is 3, w is 0, x is 1, y is 1, z is 7, and a is 1; F₁ is methionine; the string of three (3) F₂ residues is as follows: K-S-S; F₃ is absent; the string of fourteen (14) residues given by [(F₄)₁-(F₅)₁]₇ is as follows: S-L-L-L-L-A-L-L-A-L-L-A-A-L (SEQ ID NO: 96); F₆ is A; F₇ is S; F₈ is A; F₉ is present and is A; and F₁₀ is present and is P.

### Variant of SEQ ID NO.s 70, 71, 72, and 73 (Formula XIII)

In some embodiments, the pre-protein signal peptide comprises an amino acid sequence represented by:

L₁-(L₂)ₓ-[(L₃)ₐ-(L₄)ₐ]_{y}-[(L₅)ₐ-(L₆)ₐ-(L₇)ₐ]_{z}-(L₈)ₐ-(L₉)ₐ-(L₁₀)ₐ-(L₁₁)ₐ-(L₁₂)ₐ (Formula XIII)

wherein L₂-L₁₂ have the properties described in Table 9 below:

TABLE 9

| | Isoelectric | Molecular | HP | |
|---|---|---|---|---|
| AA Label | Point | Weight | Index | Helicity |
| L₂ | 2.7-10.8 | 75-205 | −5.1-34 | 0.5-1.3 |
| L₃ and L₆ | 2.7-10.8 | 75-205 | −5.1-34 | 0.5-1.3 |
| L₄, L₇, | 2.7-10.8 | 75-205 | −5.1-34 | 0.5-1.3 |
| and L₉ | | | | |
| L₅, L₈, L₁₀, | 2.7-10.8 | 75-205 | −5.1-34 | 0.5-1.3 |
| and L₁₁ | | | | |
| L₁₂ | 2.7-10.8 | 75-205 | −5.1-34 | 0.5-1.3 |

wherein:
x is 1, 2, or 3;
y is 1, 2, 3, or 4;
z is 5, 6, 7, 8, 9, or 10; and
each a is, independently, 0 or 1.

In some embodiments, L₁ is methionine. In some embodiments, each L₂ is, independently, an amino acid having an isoelectric point of about 2.7 to about 10.8, a molecular weight of about 75 g/mol to about 205 g/mol; a hydropathy index of about −5.1 to about 34, and a helicity or about 0.5 to about 1.3. In some embodiments, each L₃ and L₆ is each, independently, an amino acid having an isoelectric point of about 2.7 to about 10.8, a molecular weight of about 75 g/mol to about 205 g/mol; a hydropathy index of about −5.1 to about 34, and a helicity or about 0.5 to about 1.3. In some embodiments, each L₃ is each, independently, an amino acid having an isoelectric point of about 2.7 to about 10.8, a molecular weight of about 75 g/mol to about 205 g/mol; a hydropathy index of about −5.1 to about 34, and a helicity or about 0.5 to about 1.3. In some embodiments, each L₆ is each, independently, an amino acid having an isoelectric point of about 2.7 to about 10.8, a molecular weight of about 75 g/mol to about 205 g/mol; a hydropathy index of about −5.1 to about 34, and a helicity or about 0.5 to about 1.3. In some embodiments, each L₄, L₇ and L₉ is each, independently, an amino acid having an isoelectric point of about 2.7 to about 10.8, a molecular weight of about 75 g/mol to about 205 g/mol; a hydropathy index of about −5.1 to about 34, and a helicity or about 0.5 to about 1.3. In some embodiments, each L₄ is each, independently, an amino acid having an isoelectric point of about 2.7 to about 10.8, a molecular weight of about 75 g/mol to about 205 g/mol; a hydropathy index of about −5.1 to about 34, and a helicity or about 0.5 to about 1.3. In some embodiments, each L₇ is each, independently, an amino acid having an isoelectric point of about 2.7 to about 10.8, a molecular weight of about 75 g/mol to about 205 g/mol; a hydropathy index of about −5.1 to about 34, and a helicity or about 0.5 to about 1.3. In some embodiments, L₉ is an amino acid having an isoelectric point of about 2.7 to about 10.8, a molecular weight of about 75 g/mol to about 205 g/mol; a hydropathy index of about −5.1 to about 34, and a helicity or about 0.5 to about 1.3. In some embodiments, each L₅, L₈, L₁₀ and L₁₁ is each, independently, an amino acid having an isoelectric point of about 2.7 to about 10.8, a molecular weight of about 75 g/mol to about 205 g/mol; a hydropathy index of about −5.1 to about 34, and a helicity or about 0.5 to about 1.3. In some embodiments, each L₅ is each, independently, an amino acid having an isoelectric point of about 2.7 to about 10.8, a molecular weight of about 75 g/mol to about 205 g/mol; a hydropathy index of about −5.1 to about 34, and a helicity or about 0.5 to about 1.3. In some embodiments, L₈ is an amino acid having an isoelectric point of about 2.7 to about 10.8, a molecular weight of about 75 g/mol to about 205 g/mol; a hydropathy index of about −5.1 to about 34, and a helicity or about 0.5 to about 1.3. In some embodiments, L₁₀ is an amino acid having an isoelectric point of about 2.7 to about 10.8, a molecular weight of about 75 g/mol to about 205 g/mol; a hydropathy index of about −5.1 to about 34, and a helicity or about 0.5 to about 1.3. In some embodiments, L₁₁ is an amino acid having an isoelectric point of about 2.7 to about 10.8, a molecular weight of about 75 g/mol to about 205 g/mol; a hydropathy index of about −5.1 to about 34, and a helicity or about 0.5 to about 1.3. In some embodiments, L₁₂ is an amino acid having an isoelectric point of about 2.7 to about 10.8, a molecular weight of about 75 g/mol to about 205 g/mol; a hydropathy index of about −5.1 to about 34, and a helicity or about 0.5 to about 1.3.

In some embodiments, x is 1. In some embodiments, x is 2. In some embodiments, x is 3. In some embodiments, y is 1. In some embodiments, y is 2. In some embodiments, y is 3. In some embodiments, y is 4. In some embodiments, z is 5. In some embodiments, z is 6. In some embodiments, z is 7. In some embodiments, z is 8. In some embodiments, z is 9. In some embodiments, z is 10. In some embodiments, a is 0. In some embodiments, a is 1. It is to be understood that the values of any variable x, y, z, and a are each independently selected, and the value of any variable x, y, z, or a is independent of the value selected for the other variables. In some embodiments, L₁ is methionine. In some embodiments, each L₂ is, independently, an amino acid selected from the group consisting of R, K, H, S, G, N, Q, D, T, A, C, P, Y, M, V, W, I, F, and L. In some embodiments, each L₂ is, independently, an amino acid selected from the group consisting of R, K, and H. In some embodiments, L₃ is absent. In some embodiments, L₃ is present. In some embodiments, each L₃ is, independently, an amino acid selected from the group consisting of S, N, Q, R, T, K, P, G, E, H, D, A, C, Y, M, V, W, I, F, and L. In some embodiments, each L₃ is, independently, an amino acid selected from the group consisting of S, N, Q, R, T, K, and P. In some embodiments, L₄ is absent. In some embodiments, L₄ is present. In some embodiments, each L₄ is, independently, an amino acid selected from the group consisting of L, F, I, W, V, T, M, Y, P, C, A, Q, N, S, G, E, D, R, K, and H. In some embodiments, each L₄ is, independently, an amino acid selected from the group consisting of L, F, I, W, V, and T. In some embodiments, L₅ is absent. In some embodiments, L₅ is present. In some embodiments, each L₅ is, independently, an amino acid selected from the group consisting of A, T, G, S, C, P, I, L, F, R, V, Q, Y, K, N, E, D, H, M, and W. In some embodiments, each L₅ is, independently, an amino acid selected from the group consisting of A, T, G, and S. In some embodiments, L₆ is absent. In some embodiments, L₆ is present. In some embodiments, each L₆ is, independently, an amino acid selected from the group consisting of S, N, Q, R, T, K, P, G, E, H, D, A, C, Y, M, V, W, I, F, and L. In some embodiments, each L₆ is, independently, an amino acid selected from the group consisting of S, N, Q, R, T, K, and P. In some embodiments, L₇ is absent. In some embodiments, L₇ is present. In some embodiments, each L₇ is, independently, an amino acid selected from the group consisting of L, F, I, W, V, T, M, Y, P, C, A, Q, N, S, G, E, D, R, K, and H. In some embodiments, each L₇ is, independently, an amino acid selected from the group consisting of L, F, I, W, V, and T. In some embodiments, L₈ is absent. In some embodiments, L₈ is present. In some embodiments, L₈ is an amino acid selected from the group consisting of A, T, G, S, C, P, I, L, F, R, V, Q, Y, K, N, E, D, H, M, and W. In some embodiments L₈ is an amino acid selected from the group consisting of A, T, G, and S. In some embodiments, L₉ is absent. In some embodiments, L₉ is present. In some embodiments, L₉ is an amino acid selected from the group consisting of L, F, I, W, V, T, M, Y, P, C, A, Q, N, S, G, E, D, R, K, and H. In some embodiments, L₉ is an amino acid selected from the group consisting of L, F, I, W, V, and T. In some embodiments, L₁₀ is absent. In some embodiments, L₁₀ is present. In some embodiments, L₁₀ is an amino acid selected from the group consisting of A, T, G, S, C, P, I, L, F, R, V, Q, Y, K, N, E, D, H, M, and W. In some embodiments, L₁₀ is an amino acid selected from the group consisting of A, T, G, and S. In some embodiments, L₁₁ is absent. In some embodiments, L₁ is present. In some embodiments, Ln is an amino acid selected from the group consisting of A, T, G, S, C, P, I, L, F, R, V, Q, Y, K, N, E, D, H, M, and W. In some embodiments Ln is an amino acid selected from the group consisting of A, T, G, and S. In some embodiments, L₁₂ is absent. In some embodiments, L₁₂ is present. In some embodiments, L₁₂ is an amino acid selected from the group consisting of P, T, S, D, C, Y, M, V, A, Q, N, W, G, I, E, L, F, R, K, and H. In some embodiments L₁₂ is an amino acid selected from the group consisting of P, T, S, and D. In embodiments where any one of x, y, and z are an integer greater than 1, each amino acid in the group described by the x, y, and z are independently chosen from the disclosed group of amino acids and therefore may be the same or different, as described for herein.

As outlined pertaining to Formula III, the portion of Formula XIII given by [(L₅)ₐ-(L₆)ₐ-(L₇)ₐ]_{z} is not to be interpreted as “z” repeats of [(L₅)ₐ-(L₆)ₐ-(L₇)ₐ], but rather, when expanded “z” times, each a may be independently selected from an integer as provided for above, and each L₅, L₆, and L₇ may be independently selected from an appropriate amino acid as provided for above.

Thus, for example, when considering the formula of Formula XIII L₁-(L₂)ₓ-[(L₃)ₐ-(L₄)ₐ]_{y}-[(L₅)ₐ-(L₆)ₐ-(L₇)ₐ]_{z}-(L₈)ₐ-(L₉)ₐ-(L₁₀)ₐ-(L₁₁)ₐ-(L₁₂)ₐ wherein z is 5, one can also envision the formula of Formula XIII to be written as: L₁-(L₂)ₓ-[(L₃)ₐ-(L₄)ₐ]_{y}-(L₅)ₐ-(L₆)ₐ-(L₇)ₐ-(L₅)ₐ-(L₆)ₐ-(L₇)ₐ-(L₅)ₐ-(L₆)ₐ-(L₇)ₐ-(L₅)ₐ-(L₆)ₐ-(L₇)ₐ-(L₅)ₐ (L₆)ₐ-(L₇)ₐ-(L₈)ₐ-(L₉)ₐ-(L₁₀)ₐ-(L₁₁)ₐ-(L₁₂)ₐ wherein each a is, independently, 0 or 1 and each L₅, L₆, and L₇ are selected, independently, from an appropriate amino acid as outline above.

In some embodiments, the sequence of SEQ ID NO. 70 can be derived from Formula XIII as follows: x is 1, y is 2, and z is 6; L₁ is methionine; L₂ is R; all four instances of “a” within [(L₃)ₐ-(L₄)ₐ]₂ are 1 and the string of four (4) residues given by [(L₃)₁-(L₄)₁]₂ is as follows: S-L-S-L; for every (L₅)ₐ, “a” is 1; for every (L₆)ₐ, “a” is 0; for every (L₇)ₐ, “a” is 1; the string of twelve (12) residues given by [(L₅)₁-(L₇)₁]₆ is as follows: A-L-L-L-L-L-A-L-L-A-S-L (SEQ ID NO: 97); L₆ is absent; L₈ is present and is A; L₉ is present and is L; L₁₀ is present and is A; L₁₁ is present and is A; L₁₂ is present and is P.

In some embodiments, the sequence of SEQ ID NO. 71 can be derived from Formula XIII as follows: x is 1, y is 2, and z is 6; L₁ is methionine; L₂ is R; all four instances of “a” within [(L₃)ₐ-(L₄)ₐ]₂ are 1 and the string of four (4) residues given by [(L₃)₁-(L₄)₁]₂ is as follows: L-S-L-S; for every (L₅)ₐ, “a” is 1; for every (L₆)ₐ, “a” is 0; for every (L₇)ₐ, “a” is 1; the string of twelve (12) residues given by [(L₅)₁-(L₇)₁]₆ is as follows: L-L-L-L-L-L-A-L-L-A-S-L (SEQ ID NO: 98); L₆ is absent; L₈ is present and is A; L₉ is present and is L; L₁₀ is present and is A; L₁₁ is present and is A; L₁₂ is present and is P.

In some embodiments, the sequence of SEQ ID NO. 72 can be derived from Formula XIII as follows: x is 1, y is 2, and z is 6; L₁ is methionine; L₂ is R; all four instances of “a” within [(L₃)ₐ-(L₄)ₐ]₂ are 1 and the string of four (4) residues given by [(L₃)₁-(L₄)₁]₂ is as follows: L-S-S-L; for every (L₅)ₐ, “a” is 1; for every (L₆)ₐ, “a” is 0; for every (L₇)ₐ, “a” is 1; the string of twelve (12) residues given by [(L₅)₁-(L₇)₁]₆ is as follows: L-L-G-L-L-L-A-L-A-A-S-L (SEQ ID NO: 99); L₆ is absent; L₈ is present and is A; L₉ is present and is L; L₁₀ is present and is A; L₁₁ is present and is A; L₁₂ is present and is P.

In some embodiments, the sequence of SEQ ID NO. 73 can be derived from Formula XIII as follows: x is 1, y is 1, and z is 7; L₁ is methionine; L₂ is R; both instances of “a” within [(L₃)ₐ-(L₄)ₐ]₂ are 1 and the string of two (2) residues given by [(L₃)₁-(L₄)₁]₁ is as follows: L-S; for every (L₅)ₐ, “a” is 1; for every (L₆)ₐ, “a” is 0; for every (L₇)ₐ, “a” is 1; the string of fourteen (14) residues given by [(L₅)₁-(L₇)₁]₇ is as follows: L-L-L-A-L-L-A-L-L-A-L-A-S-L (SEQ ID NO: 100); L₆ is absent; L₈ is present and is A; L₉ is present and is L; L₁₀ is present and is A; L₁₁ is present and is A; L₁₂ is present and is P.

### Variants of SEQ ID NO. 21 (Formula VI)

In some embodiments, the pro-protein signal peptide comprises an amino acid sequence represented by:

G₁-G₂-G₃-G₄-G₅-G₆-G₇-G₈-G₉-G₁₀-G₁₁-G₁₂-G₁₃-G₁₄-G₁₅-G₁₆-G₁₇-G₁₈-G₁₉-G₂₀-G₂₁-G₂₂-G₂₃-G₂₄-G₂₅ (Formula VI)

wherein Table 10 below describes the various substitutions that may be made, with preferable amino acids underlined.

TABLE 10

| | Suitable | Isoelectric | Molecular | HP | |
|---|---|---|---|---|---|
| Position | Amino Acids | Point | Weight | Index | Helicity |
| G₁ | I, L, F, V, A, N, S, D, R, K | 2.7-10.8 | 89-175 | −3.7-31 | 0.8-1.3 |
| G₂ | P, S, N, G, E | 3.2-6.3 | 75-148 | −0.5-10 | 0.5-1.2 |
| G₃ | L, F, I, V, Y, A, S, R, H | 5.4-10.8 | 89-182 | −5.1-31 | 0.9-1.3 |
| G₄ | V, M, P, Y, A, T, S, N, K, H | 5.4-9.8 | 89-182 | −5.1-17 | 0.5-1.3 |
| G₅ | A, G, R, Y, K, D, M, V, W, I, L | 2.7-10.8 | 75-205 | −4-34 | 0.8-1.3 |
| G₆ | N, R, K | 5.4-10.8 | 132-175 | −4-0 | 0.8-1 |
| G₇ | V, P, A, T, Q, G, E, D, R, K | 2.7-10.8 | 75-175 | −4-14 | 0.5-1.3 |
| G₈ | P, Y, T, Q, S, N, W, F, R, K, H | 5.4-10.8 | 105-205 | −5.1-34 | 0.5-1.3 |
| G₉ | F, L, A, Q, N, S, E, G, D, H | 2.7-8.6 | 75-166 | −5.1-31 | 0.8-1.3 |
| G₁₀ | H, S, N, D, Q, E, T, Y, | 2.7-8.6 | 105-182 | −5.1-25 | 0.8-1.3 |
| | M, V, I, L | | | | |
| G₁₁ | S, R, T, G, K, E, D, P | 2.7-10.8 | 75-175 | −4-10 | 0.5-1.15 |
| G₁₂ | D, E, Q, N, A, V | 2.7-6 | 89-148 | −1-14 | 0.8-1.3 |
| G₁₃ | N, S, E, D, T, H, K, A, P | 2.7-9.8 | 89-156 | −5.1-10 | 0.5-1.25 |
| G₁₄ | G, S, N, H, E, C, Y, L, F | 3.2-8.6 | 75-182 | −5.1-31 | 0.7-1.3 |
| G₁₅ | S, T, H | 5.6-8.6 | 105-156 | −5.1-3 | 0.9-1.1 |
| G₁₆ | E, D, Q, N, S, T, K, A | 2.7-9.8 | 89-148 | −4-4 | 0.8-1.25 |
| G₁₇ | W, N, D, R | 2.7-10.8 | 132-205 | −4-34 | 0.8-1.1 |
| G₁₈ | L, F | 5.4-6 | 131-166 | 24-31 | 1.2-1.3 |
| G₁₉ | Y, V, A, Q, N, S, E, D, | 2.7-10.8 | 89-182 | −5.1-25 | 0.8-1.3 |
| | L, R, K, H | | | | |
| G₂₀ | K, R, S, I | 5.6-10.8 | 105-175 | −4-23 | 0.8-1.3 |
| G₂₁ | R | — | — | — | — |
| G₂₂ | D, E, N, S, T, G, A, Y, L | 2.7-6 | 75-182 | −1-25 | 0.8-1.3 |
| G₂₃, G₂₄ | V, P, Y, I, A, E, K, F, T, | 2.7-9.8 | 75-182 | −4-31 | 0.5-1.3 |
| | S, G, D, M, N | | | | |
| G₂₅ | Y, P, A, T, Q, S, E, F, H | 3.22-8.6 | 89-182 | −5-31 | 0.5-1.3 |

In some embodiments, G₁ is an amino acid selected from the group consisting of I, L, F, V, A, N, S, D, R, and K. In some embodiments, G₂ is an amino acid selected from the group consisting of P, S, N, G, and E. In some embodiments, G₃ is an amino acid selected from the group consisting of L, F, I, V, Y, A, S, R, and H. In some embodiments, G₄ is an amino acid selected from the group consisting of V, M, P, Y, A, T, S, N, K, and H. In some embodiments, G₅ is an amino acid selected from the group consisting of A, G, R, Y, K, D, M, V, W, I, and L. In some embodiments, G₆ is an amino acid selected from the group consisting of N, R, and K. In some embodiments, G₇ is an amino acid selected from the group consisting of V, P, A, T, Q, G, E, D, R, and K. In some embodiments, G₈ is an amino acid selected from the group consisting of P, Y, T, Q, S, N, W, F, R, K, and H. In some embodiments, G₉ is an amino acid selected from the group consisting of F, L, A, Q, N, S, E, G, D, and H. In some embodiments, G₁₀ is an amino acid selected from the group consisting of H, S, N, D, Q, E, T, Y, M, V, I, and L. In some embodiments, G₁₁ is an amino acid selected from the group consisting of S, R, T, G, K, E, D, and P. In some embodiments, G₁₂ is an amino acid selected from the group consisting of D, E, Q, N, A, and V. In some embodiments, G₁₃ is an amino acid selected from the group consisting of N, S, E, D, T, H, K, A, and P. In some embodiments, G₁₄ is an amino acid selected from the group consisting of G, S, N, H, E, C, Y, L, and F. In some embodiments, G₁₅ is an amino acid selected from the group consisting of S, T, and H. In some embodiments, G₁₆ is an amino acid selected from the group consisting of E, D, Q, N, S, T, K, and A. In some embodiments, G₁₇ is an amino acid selected from the group consisting of W, N, D, and R. In some embodiments, G₁₈ is an amino acid selected from the group consisting of L and F. In some embodiments, G₁₉ is an amino acid selected from the group consisting of Y, V, A, Q, N, S, E, D, L, R, K, and H. In some embodiments, G₂₀ is an amino acid selected from the group consisting of K, R, S, and I. In some embodiments, G₂₁ is R. In some embodiments, G₂₂ is an amino acid selected from the group consisting of D, E, N, S, T, G, A, Y, and L. In some embodiments, G₂₃ is an amino acid selected from the group consisting of V, P, Y, I, A, E, K, F, T, S, G, D, M, and N. In some embodiments, G₂₃ is an amino acid selected from the group consisting of V, P, Y, I, A, E, and K. In some embodiments, G₂₄ is an amino acid selected from the group consisting of V, P, Y, I, A, E, K, F, T, S, G, D, M, and N. In some embodiments, G₂₄ is an amino acid selected from the group consisting of V, P, Y, I, A, E, and K. In some embodiments, G₂₅ is an amino acid selected from the group consisting of Y, P, A, T, Q, S, E, F, and H.

In some embodiments, the pro-protein signal peptide comprises an amino acid sequence of SEQ ID NO. 86 (IPLVANVSFNSDNGSQWLYKRDVVY).

### Variants of SEQ ID NOs. 22, 23, and 24 (Formula VII and Formula VIII)

In some embodiments, the pro-protein signal peptide comprises an amino acid sequence represented by:

(H₁)ₘ-(H₂)ₘ-(H₃)ₘ-(H₄)ₘ-(H₅)ₘ-(H₆)ₘ-(H₇)ₘ-(H₈)ₘ-(H₉)ₘ-(H₁₀)ₘ-(H₁₁)ₘ- (H₁₂)ₘ-(H₁₃)ₘ-(H₁₄)ₘ-(H₁₅)ₘ-(H₁₆)ₘ-(H₁₇)ₘ-(H₁₈)ₘ-(H₁₉)ₘ-(H₂₀)ₘ-(H₂₁)ₘ-(H₂₂)ₘ-(H₂₃)ₘ-(H₂₄)ₘ-(H₂₅)ₘ-(H₂₆)ₘ-(H₂₇)ₘ-(H₂₈)ₘ-(H₂₉)ₘ-(H₃₀)ₘ-(H₃₁)ₘ-(H₃₂)ₘ-(H₃₃)ₘ-(H₃₄)ₘ-(H₃₅)ₘ-(H₃₆)ₘ-H₃₇-H₃₈-H₃₉-H₄₀ (Formula VII)

wherein each m is, independently, 0, 1, or 2. Table 11 below describes the various amino acids that may be used at each position, with preferable amino acids underlined.

TABLE 11

| | Suitable | Isoelectric | Molecular | HP | |
|---|---|---|---|---|---|
| Position | Amino Acids | Point | Weight | Index | Helicity |
| H₁ | E, D, S, L, G, Q, A | 2.7-6 | 105-148 | −1-25 | 0.8-1.3 |
| H₂, H₂₈ | P, S. R, T, N, G, D, K, A | 2.6-11 | 75-175 | −4-10 | 0.5-1.25 |
| H₃ | W, Y | 5.6-6 | 181-205 | 15-35 | 1-1.15 |
| H₄ | S, N, A, P, V | 5.4-6.5 | 89-135 | 0-14 | 0.5-1.3 |
| H₅, H₃₀ | T, Q, A, E, F, S | 3.2-6 | 89-210 | −0.5-31 | 0.8-1.3 |
| H₆ | L, F, I | 5.45-6.1 | 131-170 | 22-31 | 1.2-1.3 |
| H₇ | F, V, M, T, S, K | 5.45-10 | 105-170 | −4-31 | 0.8-1.3 |
| H₈ | V, P, I, A, S, K | 5.65-10 | 89-150 | −4-23 | 0.5-1.3 |
| H₉, H₁₇ | T, G, V, W, A | 5.6-6 | 75-205 | 0-34 | 1-1.3 |
| H₁₀ | R, H, S, G, N, E, T, V | 3.2-11 | 75-175 | −5.1-14 | 0.8-1.3 |
| H₁₁ | S, G, D, A, M | 2.7-6 | 75-150 | −1-17 | 0.8-1.25 |
| H₁₂ | T, S, E, G, D, K, H | 2.7-10 | 75-156 | −5.1-3 | 0.8-1.2 |
| H₁₃ | L, M, Y, N, S, D, K | 2.7-10 | 105-181 | −4-25 | 0.8-1.3 |
| H₁₄ | D, Q, N, S, K, C | 2.7-10 | 105-150 | −4-8 | 0.75-1 |
| H₁₅ | E, S, D, L, G | 3.2-6 | 75-148 | −0.5-0 | 0.8-1.2 |
| H₁₆ | I, L, V, M, A, T | 5.6-6.1 | 89-150 | 2.8-25 | 1-1.3 |
| H₁₈ | D, E, S, T, K, G | 2.7-10 | 75-148 | −4-3 | 0.8-1.2 |
| H₁₉ | Y, F, L | 5.45-6 | 131-181 | 15-31 | 1.1-1.3 |
| H₂₀ | N, Q, S, T, R, F | 5.4-11 | 105-175 | −4-31 | 0.9-1.3 |
| H₂₁, H₃₄ | S, K, T, A, Y, M, F | 5.4-10 | 89-181 | −4-31 | 0.8-1.3 |
| H₂₂ | T, Q, S, D, C, V, L | 2.7-6 | 105-150 | −1-25 | 0.75-1.3 |
| H₂₃ | G, S, K, N, H, D, W, L | 2.7-10 | 75-205 | −5.1-34 | 0.8-1.3 |
| H₂₄ | I, L, V, P, N, E | 3.2-6.5 | 115-148 | −0.5-25 | 0.5-1.3 |
| H₂₅, H₃₃ | A, T, G, R, Y, L, F, E | 3.2-11 | 75-181 | −4-31 | 0.8-1.3 |
| H₂₆, H₄₀ | V, I, F, M, L, A, T | 5.45-6.1 | 89-170 | 2.8-31 | 1-1.3 |
| H₂₇ | D, E, Q, N, S, A, I | 2.7-6.1 | 89-148 | −1-23 | 0.8-1.3 |
| H₂₉ | E, D, T, A, Y, M, V, I, F, L | 2.7-6.1 | 89-181 | −1-31 | 0.8-1.3 |
| H₃₁ | F, W, V, M, S, G, R | 5.45-11 | 75-205 | −4-34 | 0.9-1.30 |
| H₃₂ | H, S, E, G, T | 3.2-8 | 75-156 | −5.5-3 | 0.8-1.2 |
| H₃₅ | R, K, S, Q | 5.65-11 | 105-175 | −4-1 | 0.8-1 |
| H₃₆ | H, R, S, T, A, V, W, L | 5.6-11 | 89-205 | −5.1-34 | 0.9-1.3 |
| H₃₇ | K, Q, D, A, I | 2.7-10 | 89-150 | −4-23 | 0.8-1.3 |
| H₃₈ | R, K, T, F | 5.45-11 | 119-175 | −4-31 | 0.8-1.3 |
| H₃₉ | D, N, S, T, K, A, Y, L | 2.7-10 | 89-181 | −4-25 | 0.8-1.3 |

In some embodiments, amino acid positions H₁-H₃₆ may be omitted or repeated up to 1 extra time (i.e., be included 0 to 2 times), each repeat being independently selected from the indicated amino acids. Further, it is to be understood that the omission or repetition of any amino acid positions H₁-H₃₆ is independent of the omission or repetition of any amino acid at an alternate position. In some embodiments, the minimum length of a sequence generated with Formula VII is fourteen (14) amino acids.

In some embodiments, each H₁ is, independently, absent. In some embodiments, each H₁ is, independently, an amino acid selected from the group consisting of E, D, S, L, G, Q, and A. In some embodiments, each H₁ is, independently, an amino acid selected from the group consisting of E, D, and S. In some embodiments, each H₂ is, independently, absent. In some embodiments, each H₂ is, independently, an amino acid selected from the group consisting of P, S, R, T, N, G, D, K, and A. In some embodiments, each H₂ is, independently, an amino acid selected from the group consisting of P, S, and R. In some embodiments, each H₃ is, independently, absent. In some embodiments, each H₃ is, independently, an amino acid selected from the group consisting of W and Y. In some embodiments, each H₄ is, independently, absent. In some embodiments, each H₄ is, independently, an amino acid selected from the group consisting of S, N, A, P, and V. In some embodiments, each H₅ is, independently, absent. In some embodiments, each H₅ is, independently, an amino acid selected from the group consisting of T, Q, A, E, F, and S. In some embodiments, each H₅ is, independently, T. In some embodiments, each H₆ is, independently, absent. In some embodiments, each H₆ is, independently, an amino acid selected from the group consisting of L, F, and I. In some embodiments, each H₇ is, independently, absent. In some embodiments, each H₇ is, independently, an amino acid selected from the group consisting of F, V, M, T, S, and K. In some embodiments, each H₈ is, independently, absent. In some embodiments, each H₈ is, independently, an amino acid selected from the group consisting of V, P, I, A, S, and K. In some embodiments, each H₉ is, independently, absent. In some embodiments, each H₉ is, independently, an amino acid selected from the group consisting of T, G, V, W, and A. In some embodiments, each H₉ is, independently, an amino acid selected from the group consisting of T, G, and V. In some embodiments, each H₁₀ is, independently, absent. In some embodiments, each H₁₀ is, independently, an amino acid selected from the group consisting of R, H, S, G, N, E, T, and V. In some embodiments, each H₁₁ is, independently, absent. In some embodiments, each H₁₁ is, independently, an amino acid selected from the group consisting of S, G, D, A, and M. In some embodiments, each H₁₂ is, independently, absent. In some embodiments, each H₁₂ is, independently, an amino acid selected from the group consisting of T, S, E, G, D, K, and H. In some embodiments, each H₁₃ is, independently, absent. In some embodiments, each H₁₃ is, independently, an amino acid selected from the group consisting of L, M, Y, N, S, D, and K. In some embodiments, each H₁₄ is, independently, absent. In some embodiments, each H₁₄ is, independently, an amino acid selected from the group consisting of D, Q, N, S, K, and C. In some embodiments, each His is, independently, absent. In some embodiments, each His is, independently, an amino acid selected from the group consisting of E, S, D, L, and G. In some embodiments, each His is, independently, an amino acid selected from the group consisting of E and S. In some embodiments, each H₁₆ is, independently, absent. In some embodiments, each H₁₆ is, independently, an amino acid selected from the group consisting of I, L, V, M, A, and T. In some embodiments, each H₁₇ is, independently, absent. In some embodiments, each H₁₇ is, independently, an amino acid selected from the group consisting of T, G, V, W, and A. In some embodiments, each H₁₇ is, independently, an amino acid selected from the group consisting of T, G, and V. In some embodiments, each H₁₈ is, independently, absent. In some embodiments, each H₁₈ is, independently, an amino acid selected from the group consisting of D, E, S, T, K, and G. In some embodiments, each H₁₉ is, independently, absent. In some embodiments, each H₁₉ is, independently, an amino acid selected from the group consisting of Y, F, and L. In some embodiments, each H₂₀ is, independently, absent. In some embodiments, each H₂₀ is, independently, an amino acid selected from the group consisting of N, Q, S, T, R, and F. In some embodiments, each H₂₁ is, independently, absent. In some embodiments, each H₂₁ is, independently, an amino acid selected from the group consisting of S, K, T, A, Y, M, and F. In some embodiments, each H₂₁ is, independently, an amino acid selected from the group consisting of S and K. In some embodiments, each H₂₂ is, independently, absent. In some embodiments, each H₂₂ is, independently, an amino acid selected from the group consisting of T, Q, S, D, C, V, and L. In some embodiments, each H₂₃ is, independently, absent. In some embodiments, each H₂₃ is, independently, an amino acid selected from the group consisting of G, S, K, N, H, D, W, and L. In some embodiments, each H₂₄ is, independently, absent. In some embodiments, each H₂₄ is, independently, an amino acid selected from the group consisting of I, L, V, P, N, and E. In some embodiments, each H₂₅ is, independently, absent. In some embodiments, each H₂₅ is, independently, an amino acid selected from the group consisting of A, T, G, R, Y, L, F, and E. In some embodiments, each H₂₅ is, independently, A. In some embodiments, each H₂₆ is, independently, absent. In some embodiments, each H₂₆ is, independently, an amino acid selected from the group consisting of V, I, F, M, L, A, and T. In some embodiments, each H₂₆ is, independently, an amino acid selected from the group consisting of V, I, and F. In some embodiments, each H₂₇ is, independently, absent. In some embodiments, each H₂₇ is, independently, an amino acid selected from the group consisting of D, E, Q, N, S, A, and I. In some embodiments, each H₂₈ is, independently, absent. In some embodiments, each H₂₈ is, independently, an amino acid selected from the group consisting of P, S, R, T, N, G, D, K, and A. In some embodiments, each H₂₈ is, independently, an amino acid selected from the group consisting of P, S, and R. In some embodiments, each H₂₉ is, independently, absent. In some embodiments, each H₂₉ is, independently, an amino acid selected from the group consisting of E, D, T, A, Y, M, V, I, F, and L. In some embodiments, each H₃₀ is, independently, absent. In some embodiments, each H₃₀ is, independently, an amino acid selected from the group consisting of T, Q, A, E, F, and S. In some embodiments, each H₃₀ is, independently, T. In some embodiments, each H₃₁ is, independently, absent. In some embodiments, each H₃₁ is, independently, an amino acid selected from the group consisting of F, W, V, M, S, G, and R. In some embodiments, each H₃₂ is, independently, absent. In some embodiments, each H₃₂ is, independently, an amino acid selected from the group consisting of H, S, E, G, and T. In some embodiments, each H₃₃ is, independently, absent. In some embodiments, each H₃₃ is, independently, an amino acid selected from the group consisting of A, T, G, R, Y, L, F, and E. In some embodiments, each H₃₃ is, independently, A. In some embodiments, each H₃₄ is, independently, absent. In some embodiments, each H₃₄ is, independently, an amino acid selected from the group consisting of S, K, T, A, Y, M, and F. In some embodiments, each H₃₄ is, independently, an amino acid selected from the group consisting of S and K. In some embodiments, each H₃₅ is, independently, absent. In some embodiments, each H₃₅ is, independently, an amino acid selected from the group consisting of R, K, S, and Q. In some embodiments, each H₃₆ is, independently, absent. In some embodiments, each H₃₆ is, independently, an amino acid selected from the group consisting of H, R, S, T, A, V, W, and L. In some embodiments, H₃₇ is an amino acid selected from the group consisting of K, Q, D, A, and I. In some embodiments, H₃₈ is an amino acid selected from the group consisting of R, K, T, and F. In some embodiments, H₃₉ is an amino acid selected from the group consisting of D, N, S, T, K, A, Y, and L. In some embodiments, H₄₀ is an amino acid selected from the group consisting of V, I, F, M, L, A, and T. In some embodiments, H₄₀ is an amino acid selected from the group consisting of V, I, and F.

In some embodiments, the pro-protein signal peptide comprises an amino acid sequence selected from the group consisting of SEQ ID NOs. 22, 23, and 24.

In some embodiments, the pro-protein signal peptide comprises an amino acid sequence represented by:

(I₁)ₘ-(I₂)ₘ-(I₃)ₘ-(I₄)ₘ-(I₅)ₘ-(I₆)ₘ-(I₇)ₓ-(I₈)ₘ-(I₉)ₘ-(I₁₀)ₘ-(I₁₁)ₓ- (I₁₂)ₘ-(I₁₃)ₓ-(I₁₄)ₓ-(I₁₅)ₘ-(I₁₆)ₓ-(I₁₇)ₘ-I₁₈-I₁₉-I₂₀-I₂₁-I₂₂-I₂₃ (Formula VIII)

wherein each m is, independently, 0, 1, or 2 and each x is, independently, 0, 1, 2, 3, or 4. Table 12 below describes the various amino acids that may be used at each position, with preferable amino acids underlined.

TABLE 12

| | Suitable | Isoelectric | Molecular | HP | |
|---|---|---|---|---|---|
| Position | Amino Acids | Point | Weight | Index | Helicity |
| I₁, I₆ | S, Q, E, A, I, G, V, R, T, Y | 3.2-11 | 75-181 | −4-23 | 0.8-1.3 |
| I₂ | T, S, E, R, P, V, I, F | 3.2-11 | 105-175 | −4-31 | 0.5-1.3 |
| I₃ | L | — | — | — | — |
| I₄ | T, N, K, M | 5.4-10 | 119-149 | −4-17 | 0.85-1.25 |
| I₅ | P, A, D | 2.7-6.5 | 89-133 | −1-10 | 0.5-1.25 |
| I₇ | T, S, K, H, Y, V, F | 5.4-10 | 105-181 | −5.1-31 | 0.85-1.3 |
| 1₈, I₁₅ | F, L, W, A, T, M, Y, C | 5-6 | 89-204 | 2.8-34 | 0.75-1.3 |
| I₉ | I, L, V | 5.4-6.1 | 89-132 | 0-25 | 0.9-1.3 |
| I₁₀, I₁₆ | G, S, N, E, D, A, K, H, C, | 2.7-10 | 75-165 | −5.1-31 | 0.5-1.3 |
| | P, F | | | | |
| I₁₁ | I, L, V, A, T, S | 5.6-6.1 | 89-132 | 0-25 | 1-1.3 |
| I₁₂ | T, N, A, E, G | 3.2-6 | 75-147 | −0.5-4 | 0.8-1.25 |
| I₁₃ | E, Q, S, T, R, K, A, L, D, F | 2.7-11 | 89.1-175 | −4-31 | 0.8-1.3 |
| I₁₄ | T, S, Q, F, A, G, V, I, L | 5.4-6.1 | 75-165 | 0-31 | 0.9-1.3 |
| I₁₇ | I, L, V, N, A, T, S | 5.4-6.1 | 89-132 | 0-25 | 0.9-1.3 |
| I₁₈, I₂₁ | R, K, Q, A | 5.6-11 | 89-175 | −4-4 | 0.85-1.25 |
| I₁₉ | H, R, S, N, T, A, V, W | 5.4-11 | 89-204 | −5.1-34 | 0.9-1.3 |
| I₂₀ | K, N, Q, D, E, A, I | 2.7-10 | 89-147 | −4-23 | 0.8-1.3 |
| I₂₂ | D, N, S, A, Y, L | 2.7-6 | 89-181 | −1-25 | 0.85-1.3 |
| I₂₃ | V, I, L, F, A | 5.4-6.1 | 89-165 | 3.3-31 | 1.2-1.3 |

In some embodiments, amino acid positions I₁-I₆, I₈, I₉, I₁₂, I₁₅, and I₁₇ may be omitted or repeated up to 1 extra time (i.e., be included 0 to 2 times), each repeat being independently selected from the indicated amino acids. In some embodiments, amino acid positions I₇, I₁₁, I₁₃, I₁₄, and I₁₆ may be omitted or repeated up to 3 extra time (i.e., be included 0 to 4 times), each repeat being independently selected from the indicated amino acids. Further, it is to be understood that the omission or repetition of any amino acid positions 1-9 and 11-17 is independent of the omission or repetition of any amino acid at an alternate position. In some embodiments, the minimum length of a sequence generated using Formula VIII is 17 amino acids.

In some embodiments, each I₁ is, independently, absent. In some embodiments, each I₁ is, independently, an amino acid selected from the group consisting of S, Q, E, A, I, G, V, R, T, and Y. In some embodiments, each I₁ is, independently, an amino acid selected from the group consisting of A, Q, and E. In some embodiments, each I₂ is, independently, absent. In some embodiments, each I₂ is, independently, an amino acid selected from the group consisting of T, S, E, R, P, V, I, and F. In some embodiments, each I₃ is, independently, absent. In some embodiments, each I₃ is, independently, L. In some embodiments, each I₄ is, independently, absent. In some embodiments, each I₄ is, independently, an amino acid selected from the group consisting of T, N, K, and M. In some embodiments, each I₅ is, independently, absent. In some embodiments, each I₅ is, independently, an amino acid selected from the group consisting of P, A, and D. In some embodiments, each I₆ is, independently, absent. In some embodiments, each I₆ is, independently, an amino acid selected from the group consisting of S, Q, E, A, I, G, V, R, T, and Y. In some embodiments, each I₆ is, independently, an amino acid selected from the group consisting of A, Q, and E. In some embodiments, each I₇ is, independently, absent. In some embodiments, each I₇ is, independently, an amino acid selected from the group consisting of T, S, K, H, Y, V, and F. In some embodiments, each I₈ is, independently, absent. In some embodiments, each I₈ is, independently, an amino acid selected from the group consisting of F, L, W, A, T, M, Y, and C. In some embodiments, each I₈ is, independently, an amino acid selected from the group consisting of F, L, W, A, and T. In some embodiments, each I₉ is, independently, absent. In some embodiments, each I₉ is, independently, an amino acid selected from the group consisting of I, L, and V. In some embodiments, each I₁₀ is, independently, absent. In some embodiments, each I₁₀ is, independently, an amino acid selected from the group consisting of G, S, N, E, D, A, K, H, C, P, and F. In some embodiments, each I₁₀ is, independently, an amino acid selected from the group consisting of G and S. In some embodiments, each I₁₁ is, independently, absent. In some embodiments, each I₁₁ is, independently, an amino acid selected from the group consisting of I, L, V, A, T, and S. In some embodiments, each I₁₂ is, independently, absent. In some embodiments, each I₁₂ is, independently, an amino acid selected from the group consisting of T, N, A, E, and G. In some embodiments, each I₁₃ is, independently, absent. In some embodiments, each I₁₃ is, independently, an amino acid selected from the group consisting of E, Q, S, T, R, K, A, L, D, and F. In some embodiments, each I₁₃ is, independently, E. In some embodiments, each I₁₄ is, independently, absent. In some embodiments, each I₁₄ is, independently, an amino acid selected from the group consisting of T, S, Q, F, A, G, V, I, and L. In some embodiments, each I₁₄ is, independently, an amino acid selected from the group consisting of T and S. In some embodiments, each I₁₅ is, independently, absent. In some embodiments, each I₁₅ is, independently, an amino acid selected from the group consisting of F, L, W, A, T, M, Y, and C. In some embodiments, each I₁₅ is, independently, an amino acid selected from the group consisting of F, L, W, A, and T. In some embodiments, each I₁₆ is, independently, absent. In some embodiments, each I₁₆ is, independently, an amino acid selected from the group consisting of G, S, N, E, D, A, K, H, C, P, and F. In some embodiments, each I₁₆ is, independently, an amino acid selected from the group consisting of G and S. In some embodiments, each I₁₇ is, independently, absent. In some embodiments, each I₁₇ is, independently, an amino acid selected from the group consisting of I, L, V, N, A, T, and S. In some embodiments, each I₁₇ is, independently, an amino acid selected from the group consisting of I, L, and V. In some embodiments, I₁₈ is an amino acid selected from the group consisting of R, K, Q, and A. In some embodiments, I₁₈ is R. In some embodiments, I₁₉ is an amino acid selected from the group consisting of H, R, S, N, T, A, V, and W. In some embodiments, I₂₀ is an amino acid selected from the group consisting of K, N, Q, D, E, A, and I. In some embodiments, I₂₁ is an amino acid selected from the group consisting of R, K, Q, and A. In some embodiments, I₂₁ is R. In some embodiments, I₂₂ is an amino acid selected from the group consisting of D, N, S, A, Y, and L. In some embodiments, I₂₃ is an amino acid selected from the group consisting of V, I, L, F, and A.

### Variants of Primary SEQ ID NOs. 34, 35, 36, 37, and 38 (Formula X)

In some embodiments, the pro-protein signal peptide comprises an amino acid sequence represented by:

(J₁)_{z}-(J₂)_{z}-(J₃)_{z}-(J₄)_{z}-(J₅)_{z}-(J₆)_{z}-(J₇)_{z}-(J₈)_{z}-(J₉)_{z}-(J₁₀)_{z}-(J₁₁)_{z}- (J₁₂)_{z}-(J₁₃)_{z}-(J₁₄)_{z}-(J₁₅)_{z}-(J₁₆)_{z}-(J₁₇)_{z}-(J₁₈)_{z}-(J₁₉)_{z}-(J₂₀)_{z}-(J₂₁)_{z}-J₂₂-J₂₃-J₂₄-J₂₅ (Formula X)

wherein each z is, independently, 0, 1, 2, 3, 4, or 5. Table 13 below describes the various amino acids that may be used at each position, with preferable amino acids underlined.

TABLE 13

| | Suitable | Isoelectric | Molecular | HP | |
|---|---|---|---|---|---|
| Position | Amino Acids | Point | Weight | Index | Helicity |
| J₁ | H, K, G, A, P, F, L | 5.4-10 | 75-166 | −5.1-31 | 0.5-1.3 |
| J₂ | D, E, N, G, P, H, T, R, K, | 2.7-11 | 75-175 | −5.1-10 | 0.5-1.25 |
| | A | | | | |
| J₃ | G, A, P, V, L | 5.9-6.3 | 75-132 | 0-25 | 0.5-1.3 |
| J₄ | F, I, P, A, S, E, D, R, K | 2.7-11 | 89-175 | −4-31 | 0.5-1.3 |
| J₅ | S, R, T, G, K, E, D, C | 2.7-11 | 75-175 | −4-8 | 0.75-1.15 |
| J₆ | T, S, A, D, F | 2.7-6 | 89-166 | −1-31 | 0.85-1.25 |
| J₇ | D, E, N, G, P, H, T, R, K, | 2.7-11 | 75-175 | −5.1-10 | 0.5-1.25 |
| | A | | | | |
| J₈ | Y, C, A, W, I, S, E, D, F, | 2.7-11 | 89-205 | −4-34 | 0.75-1.3 |
| | L, R, K | | | | |
| J₉ | H, K, N, D, G, T, A, C, Y, | 2.7-10 | 75-182 | −5.1-25 | 0.75-1.3 |
| | V, L | | | | |
| J₁₀ | L, V, A, G, E, I, P, R | 3.2-11 | 75-175 | −4-25 | 0.5-1.3 |
| J₁₁ | I, W, V, Y, P, T, N, S, R, | 5.4-11 | 105-205 | −4-34 | 0.5-1.3 |
| | K | | | | |
| J₁₂ | A, G, Q, N, R, Y, E, D, L | 2.7-11 | 75-182 | −4-25 | 0.85-1.3 |
| J₁₃ | I, L, W, V, M, Y, P, A, S, | 5.6-6.3 | 75-205 | 0-33 | 0.5-1.3 |
| | G | | | | |
| J₁₄ | V, C, L, F, A, T, N, G, R | 5-11 | 75-175 | −4-31 | 0.75-1.3 |
| J₁₅ | G, S, R, K, A, T, H, E, W, | 3.2-11 | 75-205 | −5.1-34 | 0.85-1.3 |
| | L, F | | | | |
| J₁₆ | D, E, Q, S, H, T, R, G, Y, | 2.7-11 | 75-182 | −5.1-31 | 0.85-1.3 |
| | V, F, L | | | | |
| J₁₇ | E, S, G, Y, I, L | 3.2-6.1 | 75-182 | −0.5-25 | 0.85-1.3 |
| J₁₈ | A, S, P, H, V | 5.6-7.6 | 89-156 | −5.1-14 | 0.5-1.3 |
| J₁₉ | N, E, R, K, A | 3.2-11 | 89-175 | −4-4 | 0.85-1.25 |
| J₂₀ | R, T, V, I, L | 5.6-11 | 117-175 | −4-25 | 0.95-1.3 |
| J₂₁ | L, V, A, G, E, I, P, R | 3.2-11 | 75-175 | −4-25 | 0.5-1.3 |
| J₂₂ | K, R, D, T, M, W | 2.7-11 | 119-205 | −4-34 | 0.85-1.25 |
| J₂₃ | R, T, V, I, L | 5.6-11 | 117-175 | −4-25 | 0.95-1.3 |
| J₂₄ | S, N, G, E, D, P, W | 2.7-6.3 | 75-205 | −1-34 | 0.5-1.15 |
| J₂₅ | A, T, S, Y, M, V, L | 5.6-6 | 89-182 | 0-25 | 1-1.3 |

In some embodiments, amino acid positions J₁-J₂₁ may be omitted or repeated up to 4 extra time (i.e., be included 0 to 5 times), each repeat being independently selected from the indicated amino acids. Further, it is to be understood that the omission or repetition of any amino acid positions J₁-J₂₁ is independent of the omission or repetition of any amino acid at an alternate position.

In some embodiments, each J₁ is, independently, absent. In some embodiments, each J₁ is, independently, an amino acid selected from the group consisting of H, K, G, A, P, F, and L. In some embodiments, each J₂ is, independently, absent. In some embodiments, each J₂ is, independently, an amino acid selected from the group consisting of D, E, N, G, P, H, T, R, K, and A. In some embodiments, each J₂ is, independently, an amino acid selected from the group consisting of D, E, N, G, and P. In some embodiments, each J₃ is, independently, absent. In some embodiments, each J₃ is, independently, an amino acid selected from the group consisting of G, A, P, V, and L. In some embodiments, each J₄ is, independently, absent. In some embodiments, each J₄ is, independently, an amino acid selected from the group consisting of F, I, P, A, S, E, D, R, and K. In some embodiments, each J₅ is, independently, absent. In some embodiments, each J₅ is, independently, an amino acid selected from the group consisting of S, R, T, G, K, E, D, and C. In some embodiments, each J₆ is, independently, absent. In some embodiments, each J₆ is, independently, an amino acid selected from the group consisting of T, S, A, D, and F. In some embodiments, each J₇ is, independently, absent. In some embodiments, each J₇ is, independently, an amino acid selected from the group consisting of D, E, N, G, P, H, T, R, K, and A. In some embodiments, each J₇ is, independently, an amino acid selected from the group consisting of D, E, N, G, and P. In some embodiments, each J₈ is, independently, absent. In some embodiments, each J₈ is, independently, an amino acid selected from the group consisting of Y, C, A, W, I, S, E, D, F, L, R, and K. In some embodiments, each J₉ is, independently, absent. In some embodiments, each J₉ is, independently, an amino acid selected from the group consisting of H, K, N, D, G, T, A, C, Y, V, and L. In some embodiments, each J₁₀ is, independently, absent. In some embodiments, each J₁₀ is, independently, an amino acid selected from the group consisting of L, V, A, G, E, I, P, and R. In some embodiments, each J₁₀ is, independently, an amino acid selected from the group consisting of L, V, A, G, and E. In some embodiments, each J₁₁ is, independently, absent. In some embodiments, each J₁₁ is, independently, an amino acid selected from the group consisting of I, W, V, Y, P, T, N, S, R, and K. In some embodiments, each J₁₂ is, independently, absent. In some embodiments, each J₁₂ is, independently, an amino acid selected from the group consisting of A, G, Q, N, R, Y, E, D, and L. In some embodiments, each J₁₃ is, independently, absent. In some embodiments, each J₁₃ is, independently, an amino acid selected from the group consisting of I, L, W, V, M, Y, P, A, S, and G. In some embodiments, each J₁₄ is, independently, absent. In some embodiments, each J₁₄ is, independently, an amino acid selected from the group consisting of V, C, L, F, A, T, N, G, and R. In some embodiments, each J₁₅ is, independently, absent. In some embodiments, each J₁₅ is, independently, an amino acid selected from the group consisting of G, S, R, K, A, T, H, E, W, L, and F. In some embodiments, each J₁₆ is, independently, absent. In some embodiments, each J₁₆ is, independently, an amino acid selected from the group consisting of D, E, Q, S, H, T, R, G, Y, V, F, and L. In some embodiments, each J₁₇ is, independently, absent. In some embodiments, each J₁₇ is, independently, an amino acid selected from the group consisting of E, S, G, Y, I, and L. In some embodiments, each J₁₈ is, independently, absent. In some embodiments, each J₁₈ is, independently, an amino acid selected from the group consisting of A, S, P, H, and V. In some embodiments, each J₁₉ is, independently, absent. In some embodiments, each J₁₉ is, independently, an amino acid selected from the group consisting of N, E, R, K, and A. In some embodiments, each J₂₀ is, independently, absent. In some embodiments, each J₂₀ is, independently, an amino acid selected from the group consisting of R, T, V, I, and L. In some embodiments, each J₂₀ is, independently, R. In some embodiments, each J₂₁ is, independently, absent. In some embodiments, each J₂₁ is, independently, an amino acid selected from the group consisting of L, V, A, G, E, I, P, and R. In some embodiments, each J₂₁ is, independently, an amino acid selected from the group consisting of L, V, A, G, and E. In some embodiments, each J₂₂ is, independently, absent. In some embodiments, J₂₂ is an amino acid selected from the group consisting of K, R, D, T, M, and W. In some embodiments, J₂₃ is an amino acid selected from the group consisting of R, T, V, I, and L. In some embodiments, J₂₄ is an amino acid selected from the group consisting of S, N, G, E, D, P, and W. In some embodiments, J₂₅ is an amino acid selected from the group consisting of A, T, S, Y, M, V, and L.

In some embodiments, the pro-protein signal peptide comprises an amino acid sequence selected from the group consisting of SEQ ID NOs. 34, 35, 36, 37, and 38.

### Variants of Primary SEQ ID NOs. 34, 35, 36, 37, and 38 (Formula XI)

In some embodiments, the pro-protein signal peptide comprises an amino acid sequence represented by:

(K₁)_{b}-(K₂)_{b}-(K₃)_{b}-(K₄)_{b}-(K₅)_{b}-(K₆)_{b}-(K₇)_{b}-(K₈)_{b}-(K₉)_{b}-(K₁₀)_{b}-(K₁₁)_{b}- (K₁₂)_{b}-(K₁₃)_{b}-(K₁₄)_{b}-(K₁₅)_{b}-(K₁₆)_{b}-(K₁₇)_{b}-(K₁₈)_{b}-(K₁₉)_{b}-(K₂₀)_{b}-(K₂₁)_{b}-(K₂₂)_{b}-(K₂₃)_{b}-(K₂₄)_{b}-(K₂₅)_{b}-(K₂₆)_{b}-(K₂₇)_{b}-(K₂₈)_{b}-(K₂₉)_{b}-(K₃₀)_{b}-(K₃₁)_{b}-(K₃₂)_{b}-(K₃₃)_{b}-(K₃₄)_{b}-(K₃₅)_{b}-(K₃₆)_{b}-(K₃₇)_{b}-(K₃₈)_{b}-(K₃₉)_{b}-(K₄₀)_{b}-(K₄₁)_{b}-(K₄₂)_{b}-(K₄₃)_{b}-(K₄₄)_{b}-(K₄₅)_{b}-(K₄₆)_{b}-(K₄₇)_{b}-(K₄₈)_{b}-(K₄₉)_{b}-(K₅₀)_{b}-(K₅₁)_{b}-(K₅₂)_{b}-(K₅₃)_{b}-(K₅₄)_{b}-(K₅₅)_{b}-(K₅₆)_{b}-(K₅₇)_{b}-(K₅₈)_{b}-(K₅₉)_{b}-(K₆₀)_{b}-(K₆₁)_{b}-(K₆₂)_{b}-(K₆₃)_{b}-(K₆₄)_{b}-(K₆₅)_{b}-(K₆₆)_{b}-(K₆₇)_{b}-(K₆₈)_{b}-(K₆₉)_{b}-(K₇₀)_{b}-(K₇₁)_{b}-(K₇₂)_{b}-(K₇₃)_{b}-(K₇₄)_{b}-(K₇₅)_{b}-(K₇₆)_{b}-(K₇₇)_{b}-(K₇₈)_{b}-(K₇₉)_{b}-(K₈₀)_{b}-(K₈₁)_{b}-(K₈₂)_{b}-(K₈₃)_{b}-(K₈₄)_{b}-(K₈₅)_{b}-(K₈₆)_{b}-(K₈₇)_{b}-(K₈₈)_{b}-K₈₉-K₈₉-K₈₉-K₈₉-K₈₉ (Formula XI)

wherein each b is, independently, 0, 1, 2, or 3. Table 14 below describes the various amino acids that may be used at each position, with preferable amino acids underlined.

TABLE 14

| | Suitable | Isoelectric | Molecular | HP | |
|---|---|---|---|---|---|
| Position | Amino Acids | Point | Weight | Index | Helicity |
| K₁ | S, G, D, A, C, P, Y | 2.7-10 | 75-182 | −4-16 | 0.5-1.25 |
| K₂ | Q, S, E, T, R, K, G, A, Y, | 3.2-11 | 75-182 | −4-23 | 0.85-1.3 |
| | M, V, I | | | | |
| K₃ | G, S, N, T, Q, D, P, L, F, | 2.7-10 | 75-166 | −4-31 | 0.5-1.3 |
| | V, K, A, C | | | | |
| K₄ | R, G, N, D, A, P, Y, L | 2.7-11 | 75-182 | −4-25 | 0.5-1.3 |
| K₅ | E, A, V, Q, G, Y, M, I, L | 3.2-6.1 | 75-182 | −0.5-25 | 0.85-1.3 |
| K₆ | S, Q, R, T, D, G, E, A, K | 2.7-11 | 75-175 | −4-4 | 0.85-1.25 |
| K₇ | N, Q, R, H, K, A, I, F, L | 5.4-11 | 89-175 | −5.1-31 | 0.85-1.3 |
| K₈ | A, T, Q, G, R, K, D, L, F, | 2.7-11 | 75-175 | −5.1-31 | 0.75-1.3 |
| | C, V, S, H | | | | |
| K₉ | G, S, N, T, Q, D, P, L, F, | 2.7-10 | 75-166 | −4-31 | 0.5-1.3 |
| | V, K, A, C | | | | |
| K₁₀ | K, H, E, A, Y, L, F | 3.2-10 | 89-182 | −5.1-31 | 0.85-1.3 |
| K₁₁ | S, T, K, E, A, C, W, F, L | 3.2-10 | 89-205 | −4-34 | 0.7-1.35 |
| K₁₂ | K, R, H, S, Q, D, E, A | 2.7-11 | 89-175 | −5.1-4 | 0.85-1.25 |
| K₁₃ | G, S, T, E, P, W, R, N, Q | 3.2-11 | 75-205 | −4-34 | 0.5-1.15 |
| K₁₄ | D, Q, S, G, V, E, N, H, R, | 2.7-11 | 75-175 | −5.1-31 | 0.5-1.3 |
| | P, F | | | | |
| K₁₅ | C, A, M, V, S, E, G, I, F, | 3.2-6.1 | 75-166 | −0.5-31 | 0.75-1.3 |
| | L | | | | |
| K₁₆ | R, K, S, Q, T, Y, N, V, I, | 5-11 | 105-182 | −4-25 | 0.75-1.3 |
| | L, C | | | | |
| K₁₇ | A, G, S, Q, Y, E, D, H, I | 2.7-7.6 | 75-182 | −5.1-23 | 0.85-1.3 |
| K₁₈ | R, K, S, Q, T, Y, N, V, I, | 5-11 | 105-182 | −4-25 | 0.75-1.3 |
| | L, C | | | | |
| K₁₉ | E, D, T, H, K, G, P, V, L | 2.7-10 | 75-156 | −5.1-25 | 0.5-1.3 |
| K₂₀ | F, L, I, V, M, T, G, R | 5.4-11 | 75-175 | −4-31 | 0.95-1.3 |
| K₂₁ | E, D, S, G, A, C, P | 2.7-6.3 | 75-148 | −1-10 | 0.5-1.25 |
| K₂₂ | D, T, G, A, Y, N, S, C, P, | 2.7-6.3 | 75-205 | −1-34 | 0.5-1.3 |
| | W, I | | | | |
| K₂₃ | G, S, N, E, D, Y, L | 2.7-6 | 75-182 | −1-25 | 0.85-1.3 |
| K₂₄ | T, S, E, G, P, I | 3.2-6.3 | 75-148 | −0.5-23 | 0.5-1.3 |
| K₂₅ | K, S, G, D, T, L | 2.7-11 | 75-182 | −4-25 | 0.85-1.3 |
| K₂₆ | S, G, K, E, D, P, F | 2.7-10 | 75-166 | −4-31 | 0.5-1.25 |
| K₂₇ | P, A, E, L, T, Q, S, G, K, | 3.2-11 | 75-205 | −4-34 | 0.5-1.3 |
| | Y, F, C, V, W, R | | | | |
| K₂₈ | E, D, Q, S, T, P, L | 2.7-6.3 | 105-148 | −1-25 | 0.5-1.3 |
| K₂₉ | A, T, S, E, V, W, I | 3.2-6.1 | 89-205 | −0.5-34 | 0.85-1.3 |
| K₃₀ | K, H, S, G, N, Q, P, Y | 5.4-10 | 75-182 | −5.1-16 | 0.5-1.15 |
| K₃₁ | L, F, V, P, A, N, G, H | 5.4-7.6 | 75-166 | −5.1-31 | 0.5-1.3 |
| K₃₂ | A, G, N, P, R, E, K | 3.2-11 | 75-175 | −4-10 | 0.5-1.25 |
| K₃₃ | R, S, N, A, P, Y, V, I, F, | 5.4-11 | 75-182 | −4-31 | 0.5-1.3 |
| | G | | | | |
| K₃₄ | E, S, T, V, I, H, A, P, F, L | 3.2-7.6 | 89-166 | −5.1-31 | 0.5-1.3 |
| K₃₅ | A, T, Q, P, R, V, N, E, L | 3.2-11 | 89-175 | −4-25 | 0.5-1.3 |
| K₃₆ | R, K, H, G, Q, D, T, Y, F | 2.7-11 | 75-182 | −5.1-31 | 0.85-1.25 |
| K₃₇ | D, E, N, T, C, Y, V, I, L | 2.7-6.1 | 117-182 | −1-25 | 0.75-1.3 |
| K₃₈ | S, Q, R, T, D, G, E, A, K | 2.7-11 | 75-175 | −4-4 | 0.85-1.25 |
| K₃₉ | K, S, G, Q, D, E, A, M, I, | 2.7-10 | 75-150 | −4-25 | 0.85-1.3 |
| | L | | | | |
| K₄₀ | H, K, S, D, E, T, P, L | 2.7-10 | 105-156 | −5.1-25 | 0.5-1.3 |
| K₄₁ | A, T, S, N, P, V, L, F | 5.4-6.3 | 89-166 | 0-31 | 0.5-1.3 |
| K₄₂ | K, D, M, V, I, L, F | 2.7-10 | 117-166 | −4-31 | 0.85-1.3 |
| K₄₃ | G, S, N, T, Q, D, P, L, F, | 2.7-10 | 75-166 | −4-31 | 0.5-1.3 |
| | V, K, A, C | | | | |
| K₄₄ | L, T, F, V, P, A, K, I | 5.4-10 | 89-166 | −4-31 | 0.5-1.3 |
| K₄₅ | G, S, K, N, T, Q, D, A, P, | 2.7-10 | 75-166 | −4-31 | 0.5-1.3 |
| | L, F, V | | | | |
| K₄₆ | L, F, Q, S, G, D | 2.7-6 | 75-166 | −1-31 | 0.85-1.3 |
| K₄₇ | S, R, E, A, P, V, W, L | 3.2-11 | 89-205 | −4-34 | 0.5-1.3 |
| K₄₈ | A, S, V, G, Q, R, E, D, L, | 2.7-11 | 75-175 | −5.1-31 | 0.75-1.3 |
| | T, K, F, C, H | | | | |
| K₄₉ | E, S, T, R, G, A, P, L | 3.2-11 | 75-175 | −4-25 | 0.5-1.3 |
| K₅₀ | S, N, R, A, P, Y | 5.4-11 | 89-182 | −4-16 | 0.5-1.25 |
| K₅₁ | G, A, T, H, M, V, L, F | 5.4-7.6 | 75-166 | −5.1-31 | 0.97-1.3 |
| K₅₂ | S, T, H, A, C, M, L | 5-7.6 | 89-156 | −5.1-25 | 0.75-1.3 |
| K₅₃ | G, S, T, E, P, W, R, N, Q | 3.2-11 | 75-205 | −4-34 | 0.5-1.15 |
| K₅₄ | S, H, Y, F, N, Q, R, T, G, | 5.4-11 | 75-182 | −5.1-31 | 0.85-1.25 |
| | K | | | | |
| K₅₅ | A, T, Q, E, M, V, I, L, F | 3.2-6.1 | 89-166 | −0.5-31 | 0.85-1.3 |
| K₅₆ | S, N, E, A, P, F, L | 3.2-6.3 | 89-166 | −0.5-31 | 0.5-1.3 |
| K₅₇ | D, S, R, K, A, V, W, I, F | 2.7-11 | 89-205 | −4-34 | 0.85-1.3 |
| K₅₈ | K, S, G, D, T, L, R, E, Y, | 2.7-11 | 75-182 | −4-25 | 0.85-1.3 |
| | N | | | | |
| K₅₉ | S, R, G, A, V, F | 5.4-11 | 75-175 | −4-31 | 0.95-1.3 |
| K₆₀ | A, T, Q, G, R, K, D, L, F, | 2.7-11 | 75-175 | −5.1-31 | 0.75-1.3 |
| | C, V, S, H | | | | |
| K₆₁ | R, S, G, N, E, T, A, V | 3.2-11 | 75-175 | −4-14 | 0.85-1.3 |
| K₆₂ | E, S, T, V, I, H, A, P, F, L | 3.2-7.6 | 89-166 | −5.1-31 | 0.5-1.3 |
| K₆₃ | A, G, S, Q, R, E, D, V, L, | 2.7-11 | 75-175 | −5.1-31 | 0.75-1.3 |
| | T, K, F, C, H | | | | |
| K₆₄ | E, A, V, Q, G, Y, M, I, L | 3.2-6.1 | 75-182 | −0.5-25 | 0.85-1.3 |
| K₆₅ | G, S, T, E, P, W, R, N, Q | 3.2-11 | 75-205 | −4-34 | 0.5-1.15 |
| K₆₆ | A, G, P, M, N, V, S | 5.4-6.3 | 75-150 | 0-17 | 0.5-1.3 |
| K₆₇ | T, Q, E, N, S, A, Y, V, | 3.2-6 | 89-205 | −0.5-34 | 0.85-1.3 |
| | W, F | | | | |
| K₆₈ | I, V, P, A | 5.9-6.3 | 89-132 | 3.3-23 | 0.5-1.3 |
| K₆₉ | D, Q, S, G, V, E, N, H, R, | 2.7-11 | 75-175 | −5.1-31 | 0.5-1.3 |
| | P, F | | | | |
| K₇₀ | G, S, R, N, T, Y, L, F | 5.4-11 | 75-182 | −4-31 | 0.9-1.3 |
| K₇₁ | E, D, N, S, T, H, Y | 2.7-7.6 | 105-182 | −5.1-16 | 0.85-1.25 |
| K₇₂ | L, I, W, V, A, T, S, E, R, | 3.2-11 | 89-205 | −4-34 | 0.85-1.3 |
| | K | | | | |
| K₇₃ | G, S, K, A, C, F, N, T, Q, | 2.7-10 | 75-166 | −4-31 | 0.5-1.3 |
| | D, P, L, V | | | | |
| K₇₄ | A, S, N, P, K, V, I, L | 5.4-10 | 89-148 | −4-25 | 0.5-1.3 |
| K₇₅ | P, A, E, L, T, Q, S, G, K, | 3.2-11 | 75-205 | −4-34 | 0.5-1.3 |
| | Y, F, C, V, W, R | | | | |
| K₇₆ | L, T, F, V, P, A, K, I | 5.4-10 | 89-166 | −4-31 | 0.5-1.3 |
| K₇₇ | M, V, Y, L, A, N, E, H | 3.2-7.6 | 89-182 | −5.1-25 | 0.85-1.3 |
| K₇₈ | D, T, G, A, Y, N, S, C, P, | 2.7-6.3 | 75-205 | −1-34 | 0.5-1.3 |
| | W, I | | | | |
| K₇₉ | A, S, V, G, Q, R, E, D, L, | 2.7-11 | 75-175 | −5.1-31 | 0.75-1.3 |
| | T, K, F, C, H | | | | |
| K₈₀ | K, R, S, A, P, V, I, L | 5.6-11 | 89-175 | −4-25 | 0.5-1.3 |
| K₈₁ | F, L, V, A, T, S, E, D, R, | 2.7-11 | 89-175 | −4-31 | 0.85-1.3 |
| | K | | | | |
| K₈₂ | L, F, M, A, N, G, E | 3.2-6 | 75-166 | −0.5-31 | 0.85-1.3 |
| K₈₃ | D, S, H, A, V, I, F, L | 2.7-7.6 | 89-166 | −5.1-31 | 0.85-1.3 |
| K₈₄ | A, T, Q, S, R, V, L, G, H, | 2.7-11 | 75-175 | −5.1-31 | 0.75-1.3 |
| | F, K, D, C | | | | |
| K₈₅ | T, Q, E, N, S, A, Y, V, | 3.2-6 | 89-205 | −0.5-34 | 0.85-1.3 |
| | W, F | | | | |
| K₈₆ | A, P, R, Y, K, D, M, L, F | 2.7-11 | 89-182 | −4-31 | 0.5-1.3 |
| K₈₇ | N, S, D, T, A, P, L | 2.7-6.3 | 89-134 | −1-25 | 0.5-1.3 |
| K₈₈ | R, S, N, A, P, Y, V, I, F, | 5.4-11 | 75-182 | −4-31 | 0.5-1.3 |
| | G | | | | |
| K₈₉ | K, R, H, G, E, T, Y, I | 3.2-11 | 75-182 | −5.1-23 | 0.85-1.3 |
| K₉₀ | R, S, G, N, Q, A, Y, W | 5.4-11 | 75-205 | −4-34 | 0.9-1.25 |
| K₉₁ | V, I, F | 5.4-6.1 | 117-166 | 14-31 | 1.25-1.3 |
| K₉₂ | A, G, P, M, N, V, S | 5.4-6.3 | 75-150 | 0-17 | 0.5-1.3 |
| K₉₃ | E, D, Q, S, R, K, M, L | 2.7-11 | 105-166 | −4-25 | 0.85-1.3 |

In some embodiments, amino acid positions K₁-K₈₈ may be omitted or repeated up to 2 extra time (i.e., be included 0 to 3 times), each repeat being independently selected from the indicated amino acids. Further, it is to be understood that the omission or repetition of any amino acid positions K₁-K₈₈ is independent of the omission or repetition of any amino acid at an alternate position.

In some embodiments, each K₁ is, independently, absent. In some embodiments, each K₁ is, independently, an amino acid selected from the group consisting of S, G, D, A, C, P, and Y. In some embodiments, each K₂ is, independently, absent. In some embodiments, each K₂ is, independently, an amino acid selected from the group consisting of Q, S, E, T, R, K, G, A, Y, M, V, and I. In some embodiments, each K₃ is, independently, absent. In some embodiments, each K₃ is, independently, an amino acid selected from the group consisting of G, S, N, T, Q, D, P, L, F, V, K, A, and C. In some embodiments, each K₃ is, independently, G. In some embodiments, each K₄ is, independently, absent. In some embodiments, each K₄ is, independently, an amino acid selected from the group consisting of R, G, N, D, A, P, Y, and L. In some embodiments, each K₅ is, independently, absent. In some embodiments, each K₅ is, independently, an amino acid selected from the group consisting of E, A, V, Q, G, Y, M, I, and L. In some embodiments, each K₅ is, independently, an amino acid selected from the group consisting of E, A, and V. In some embodiments, each K₆ is, independently, absent. In some embodiments, each K₆ is, independently, an amino acid selected from the group consisting of S, Q, R, T, D, G, E, A, and K. In some embodiments, each K₆ is, independently, an amino acid selected from the group consisting of S, Q, R, T, and D. In some embodiments, each K₇ is, independently, absent. In some embodiments, each K₇ is, independently, an amino acid selected from the group consisting of N, Q, R, H, K, A, I, F, and L. In some embodiments, each K₈ is, independently, absent. In some embodiments, each K₈ is, independently, an amino acid selected from the group consisting of A, T, Q, G, R, K, D, L, F, C, V, S, and H. In some embodiments, each K₈ is, independently, A. In some embodiments, each K₉ is, independently, absent. In some embodiments, each K₉ is, independently, an amino acid selected from the group consisting of G, S, N, T, Q, D, P, L, F, V, K, A, and C. In some embodiments, each K₉ is, independently, G. In some embodiments, each K₁₀ is, independently, absent. In some embodiments, each K₁₀ is, independently, an amino acid selected from the group consisting of K, H, E, A, Y, L, and F. In some embodiments, each K₁₁ is, independently, absent. In some embodiments, each K₁₁ is, independently, an amino acid selected from the group consisting of S, T, K, E, A, C, W, F, and L. In some embodiments, each K₁₂ is, independently, absent. In some embodiments, each K₁₂ is, independently, an amino acid selected from the group consisting of K, R, H, S, Q, D, E, and A. In some embodiments, each K₁₃ is, independently, absent. In some embodiments, each K₁₃ is, independently, an amino acid selected from the group consisting of G, S, T, E, P, W, R, N, and Q. In some embodiments, each K₁₃ is, independently, G. In some embodiments, each K₁₄ is, independently, absent. In some embodiments, each K₁₄ is, independently, an amino acid selected from the group consisting of D, Q, S, G, V, E, N, H, R, P, and F. In some embodiments, each K₁₄ is, independently, an amino acid selected from the group consisting of D, Q, S, G, and V. In some embodiments, each K₁₅ is, independently, absent. In some embodiments, each K₁₅ is, independently, an amino acid selected from the group consisting of C, A, M, V, S, E, G, I, F, and L. In some embodiments, each K₁₆ is, independently, absent. In some embodiments, each K₁₆ is, independently, an amino acid selected from the group consisting of R, K, S, Q, T, Y, N, V, I, L, and C. In some embodiments, each K₁₆ is, independently, an amino acid selected from the group consisting of R, K, S, Q, T, and Y. In some embodiments, each K₁₇ is, independently, absent. In some embodiments, each K₁₇ is, independently, an amino acid selected from the group consisting of A, G, S, Q, Y, E, D, H, and I. In some embodiments, each K₁₈ is, independently, absent. In some embodiments, each K₁₈ is, independently, an amino acid selected from the group consisting of R, K, S, Q, T, Y, N, V, I, L, and C. In some embodiments, each K₁₈ is, independently, an amino acid selected from the group consisting of R, K, S, Q, T, and Y. In some embodiments, each K₁₉ is, independently, absent. In some embodiments, each K₁₉ is, independently, an amino acid selected from the group consisting of E, D, T, H, K, G, P, V, and L. In some embodiments, each K₂₀ is, independently, absent. In some embodiments, each K₂₀ is, independently, an amino acid selected from the group consisting of F, L, I, V, M, T, G, and R. In some embodiments, each K₂₁ is, independently, absent. In some embodiments, each K₂₁ is, independently, an amino acid selected from the group consisting of E, D, S, G, A, C, and P. In some embodiments, each K₂₂ is, independently, absent. In some embodiments, each K₂₂ is, independently, an amino acid selected from the group consisting of D, T, G, A, Y, N, S, C, P, W, and I. In some embodiments, each K₂₂ is, independently, an amino acid selected from the group consisting of D, T, G, A, and Y. In some embodiments, each K₂₃ is, independently, absent. In some embodiments, each K₂₃ is, independently, an amino acid selected from the group consisting of G, S, N, E, D, Y, and L. In some embodiments, each K₂₄ is, independently, absent. In some embodiments, each K₂₄ is, independently, an amino acid selected from the group consisting of T, S, E, G, P, and I. In some embodiments, each K₂₅ is, independently, absent. In some embodiments, each K₂₅ is, independently, an amino acid selected from the group consisting of K, S, G, T, and L. In some embodiments, each K₂₆ is, independently, absent. In some embodiments, each K₂₆ is, independently, an amino acid selected from the group consisting of S, G, K, E, D, P, and F. In some embodiments, each K₂₇ is, independently, absent. In some embodiments, each K₂₇ is, independently, an amino acid selected from the group consisting of P, A, E, L, T, Q, S, G, K, Y, F, C, V, W, and R. In some embodiments, each K₂₇ is, independently, an amino acid selected from the group consisting of P and A. In some embodiments, each K₂₈ is, independently, absent. In some embodiments, each K₂₈ is, independently, an amino acid selected from the group consisting of E, D, Q, S, T, P, and L. In some embodiments, each K₂₉ is, independently, absent. In some embodiments, each K₂₉ is, independently, an amino acid selected from the group consisting of A, T, S, E, V, W, and I. In some embodiments, each K₃₀ is, independently, absent. In some embodiments, each K₃₀ is, independently, an amino acid selected from the group consisting of K, H, S, G, N, Q, P, and Y. In some embodiments, each K₃₁ is, independently, absent. In some embodiments, each K₃₁ is, independently, an amino acid selected from the group consisting of L, F, V, P, A, N, G, and H. In some embodiments, each K₃₂ is, independently, absent. In some embodiments, each K₃₂ is, independently, an amino acid selected from the group consisting of A, G, N, P, R, E, and K. In some embodiments, each K₃₃ is, independently, absent. In some embodiments, each K₃₃ is, independently, an amino acid selected from the group consisting of R, S, N, A, P, Y, V, I, F, and G. In some embodiments, each K₃₃ is, independently, an amino acid selected from the group consisting of R and S. In some embodiments, each K₃₄ is, independently, absent. In some embodiments, each K₃₄ is, independently, an amino acid selected from the group consisting of E, S, T, V, I, H, A, P, F, and L. In some embodiments, each K₃₄ is, independently, an amino acid selected from the group comprising E, S, T, V, and I. In some embodiments, each K₃₅ is, independently, absent. In some embodiments, each K₃₅ is, independently, an amino acid selected from the group consisting of A, T, Q, P, R, V, N, E, and L. In some embodiments, each K₃₅ is, independently, an amino acid selected from the group consisting of A, T, Q, P, and R. In some embodiments, each K₃₆ is, independently, absent. In some embodiments, each K₃₆ is, independently, an amino acid selected from the group consisting of R, K, H, G, Q, D, T, Y, and F. In some embodiments, each K₃₇ is, independently, absent. In some embodiments, each K₃₇ is, independently, an amino acid selected from the group consisting of D, E, N, T, C, Y, V, I, and L. In some embodiments, each K₃₈ is, independently, absent. In some embodiments, each K₃₈ is, independently, an amino acid selected from the group consisting of S, Q, R, T, D, G, E, A, and K. In some embodiments, each K₃₈ is, independently, an amino acid selected from the group consisting of S, Q, R, T, and D. In some embodiments, each K₃₉ is, independently, absent. In some embodiments, each K₃₉ is, independently, an amino acid selected from the group consisting of K, S, G, Q, D, E, A, M, I, and L. In some embodiments, each K₄₀ is, independently, absent. In some embodiments, each K₄₀ is, independently, an amino acid selected from the group consisting of H, K, S, D, E, T, P, and L. In some embodiments, each K₄₁ is, independently, absent. In some embodiments, each K₄₁ is, independently, an amino acid selected from the group consisting of A, T, S, N, P, V, L, and F. In some embodiments, each K₄₂ is, independently, absent. In some embodiments, each K₄₂ is, independently, an amino acid selected from the group consisting of K, D, M, V, I, L, and F. In some embodiments, each K₄₃ is, independently, absent. In some embodiments, each K₄₃ is, independently, an amino acid selected from the group consisting of G, S, N, T, Q, D, P, L, F, V, K, A, and C. In some embodiments, each K₄₃ is, independently, G. In some embodiments, each K₄₄ is, independently, absent. In some embodiments, each K₄₄ is, independently, an amino acid selected from the group consisting of L, T, F, V, P, A, K, and I. In some embodiments, each K₄₄ is, independently, an amino acid selected from the group consisting of L and T. In some embodiments, each K₄₅ is, independently, absent. In some embodiments, each K₄₅ is, independently, an amino acid selected from the group consisting of G, S, K, N, T, Q, D, A, P, L, F, and V. In some embodiments, each K₄₅ is, independently, G. In some embodiments, each K₄₆ is, independently, absent. In some embodiments, each K₄₆ is, independently, an amino acid selected from the group consisting of L, F, Q, S, G, and D. In some embodiments, each K₄₇ is, independently, absent. In some embodiments, each K₄₇ is, independently, an amino acid selected from the group consisting of S, R, E, A, P, V, W, and L. In some embodiments, each K₄₈ is, independently, absent. In some embodiments, each K₄₈ is, independently, an amino acid selected from the group consisting of A, S, V, G, Q, R, E, D, L, T, K, F, C, and H. In some embodiments, each K₄₈ is, independently, A. In some embodiments, each K₄₉ is, independently, absent. In some embodiments, each K₄₉ is, independently, an amino acid selected from the group consisting of E, S, T, R, G, A, P, and L. In some embodiments, each K₅₀ is, independently, absent. In some embodiments, each K₅₀ is, independently, an amino acid selected from the group consisting of S, N, R, A, P, and Y. In some embodiments, each K₅₁ is, independently, absent. In some embodiments, each K₅₁ is, independently, an amino acid selected from the group consisting of G, A, T, H, M, V, L, and F. In some embodiments, each K₅₂ is, independently, absent. In some embodiments, each K₅₂ is, independently, an amino acid selected from the group consisting of S, T, H, A, C, M, and L. In some embodiments, each K₅₃ is, independently, absent. In some embodiments, each K₅₃ is, independently, an amino acid selected from the group consisting of G, S, T, E, P, W, R, N, and Q. In some embodiments, each K₅₃ is, independently, G. In some embodiments, each K₅₄ is, independently, absent. In some embodiments, each K₅₄ is, independently, an amino acid selected from the group consisting of S, H, Y, F, N, Q, R, T, G, and K. In some embodiments, each K₅₄ is, independently, S. In some embodiments, each K₅₅ is, independently, absent. In some embodiments, each K₅₅ is, independently, an amino acid selected from the group consisting of A, T, Q, E, M, V, I, L, and F. In some embodiments, each K₅₆ is, independently, absent. In some embodiments, each K₅₆ is, independently, an amino acid selected from the group consisting of S, N, E, A, P, F, and L. In some embodiments, each K₅₇ is, independently, absent. In some embodiments, each K₅₇ is, independently, an amino acid selected from the group consisting of D, S, R, K, A, V, W, I, and F. In some embodiments, each K₅₈ is, independently, absent. In some embodiments, each K₅₈ is, independently, an amino acid selected from the group consisting of K, S, G, D, T, L, R, E, Y, and N. In some embodiments, each K₅₈ is, independently, an amino acid selected from the group consisting of K, S, G, D, T, and L. In some embodiments, each K₅₉ is, independently, absent. In some embodiments, each K₅₉ is, independently, an amino acid selected from the group consisting of S, R, G, A, V, and F. In some embodiments, each K₆₀ is, independently, absent. In some embodiments, each K₆₀ is, independently, an amino acid selected from the group consisting of A, T, Q, G, R, K, D, L, F, C, V, S, and H. In some embodiments, each K₆₀ is, independently, A. In some embodiments, each K₆₁ is, independently, absent. In some embodiments, each K₆₁ is, independently, an amino acid selected from the group consisting of R, S, G, N, E, T, A, and V. In some embodiments, each K₆₂ is, independently, absent. In some embodiments, each K₆₂ is, independently, an amino acid selected from the group consisting of E, S, T, V, I, H, A, P, F, and L. In some embodiments, each K₆₃ is, independently, absent. In some embodiments, each K₆₃ is, independently, an amino acid selected from the group consisting of A, G, S, Q, R, E, D, V, L, T, K, F, C, and H. In some embodiments, each K₆₃ is, independently, A. In some embodiments, each K₆₄ is, independently, absent. In some embodiments, each K₆₄ is, independently, an amino acid selected from the group consisting of E, A, V, Q, G, Y, M, I, and L. In some embodiments, each K₆₄ is, independently, an amino acid selected from the group consisting of E, A, and V. In some embodiments, each K₆₅ is, independently, absent. In some embodiments, each K₆₅ is, independently, an amino acid selected from the group consisting of G, S, T, E, P, W, R, N, and Q. In some embodiments, each K₆₅ is, independently, G. In some embodiments, each K₆₆ is, independently, absent. In some embodiments, each K₆₆ is, independently, an amino acid selected from the group consisting of A, G, P, M, N, V, and S. In some embodiments, each K₆₆ is, independently, an amino acid selected from the group consisting of A, G, P, and M. In some embodiments, each K₆₇ is, independently, absent. In some embodiments, each K₆₇ is, independently, an amino acid selected from the group consisting of T, Q, E, N, S, A, Y, V, W, and F. In some embodiments, each K₆₇ is, independently, an amino acid selected from the group consisting of T, Q, and E. In some embodiments, each K₆₈ is, independently, absent. In some embodiments, each K₆₈ is, independently, an amino acid selected from the group consisting of I, V, P, and A. In some embodiments, each K₆₉ is, independently, absent. In some embodiments, each K₆₉ is, independently, an amino acid selected from the group consisting of D, Q, S, G, V, E, N, H, R, P, and F. In some embodiments, each K₆₉ is, independently, an amino acid selected from the group consisting of D, Q, S, G, and V. In some embodiments, each K₇₀ is, independently, absent. In some embodiments, each K₇₀ is, independently, an amino acid selected from the group consisting of G, S, R, N, T, Y, L, and F. In some embodiments, each K₇₁ is, independently, absent. In some embodiments, each K₇₁ is, independently, an amino acid selected from the group consisting of E, D, N, S, T, H, and Y. In some embodiments, each K₇₂ is, independently, absent. In some embodiments, each K₇₂ is, independently, an amino acid selected from the group consisting of L, I, W, V, A, T, S, E, R, and K. In some embodiments, each K₇₃ is, independently, absent. In some embodiments, each K₇₃ is, independently, an amino acid selected from the group consisting of G, S, K, A, C, F, N, T, Q, D, P, L, and V. In some embodiments, each K₇₃ is, independently, G. In some embodiments, each K₇₄ is, independently, absent. In some embodiments, each K₇₄ is, independently, an amino acid selected from the group consisting of A, S, N, P, K, V, I, and L. In some embodiments, each K₇₅ is, independently, absent. In some embodiments, each K₇₅ is, independently, an amino acid selected from the group consisting of P, A, E, L, T, Q, S, G, K, Y, F, C, V, W, and R. In some embodiments, each K₇₅ is, independently, an amino acid selected from the group consisting of P and A. In some embodiments, each K₇₆ is, independently, absent. In some embodiments, each K₇₆ is, independently, an amino acid selected from the group consisting of L, T, F, V, P, A, K, and I. In some embodiments, each K₇₆ is, independently, an amino acid selected from the group consisting of L and T. In some embodiments, each K₇₇ is, independently, absent. In some embodiments, each K₇₇ is, independently, an amino acid selected from the group consisting of M, V, Y, L, A, N, E, and H. In some embodiments, each K₇₈ is, independently, absent. In some embodiments, each K₇₈ is, independently, an amino acid selected from the group consisting of D, T, G, A, Y, N, S, C, P, W, and I. In some embodiments, each K₇₈ is, independently, an amino acid selected from the group consisting of D, T, G, A, and Y. In some embodiments, each K₇₉ is, independently, absent. In some embodiments, each K₇₉ is, independently, an amino acid selected from the group consisting of A, S, V, G, Q, R, E, D, L, T, K, F, C, and H. In some embodiments, each K₇₉ is, independently, A. In some embodiments, each K₈₀ is, independently, absent. In some embodiments, each K₈₀ is, independently, an amino acid selected from the group consisting of K, R, S, A, P, V, I, and L. In some embodiments, each K₈₁ is, independently, absent. In some embodiments, each K₈₁ is, independently, an amino acid selected from the group consisting of F, L, V, A, T, S, E, D, R, and K. In some embodiments, each K₈₂ is, independently, absent. In some embodiments, each K₈₂ is, independently, an amino acid selected from the group consisting of L, F, M, A, N, G, and E. In some embodiments, each K₈₃ is, independently, absent. In some embodiments, each K₈₃ is, independently, an amino acid selected from the group consisting of D, S, H, A, V, I, F, and L. In some embodiments, each K₈₄ is, independently, absent. In some embodiments, each K₈₄ is, independently, an amino acid selected from the group consisting of A, T, Q, S, R, V, L, G, H, F, K, D, and C. In some embodiments, each K₈₄ is, independently, A. In some embodiments, each K₈₅ is, independently, absent. In some embodiments, each K₈₅ is, independently, an amino acid selected from the group consisting of T, Q, E, N, S, A, Y, V, W, and F. In some embodiments, each K₈₅ is, independently, an amino acid selected from the group consisting of T, Q, and E. In some embodiments, each K₈₆ is, independently, absent. In some embodiments, each K₈₆ is, independently, an amino acid selected from the group consisting of A, P, R, Y, K, D, M, L, and F. In some embodiments, each K₈₇ is, independently, absent. In some embodiments, each K₈₇ is, independently, an amino acid selected from the group consisting of N, S, D, T, A, P, and L. In some embodiments, each K₈₈ is, independently, absent. In some embodiments, each K₈₈ is, independently, an amino acid selected from the group consisting of R, S, N, A, P, Y, V, I, F, and G. In some embodiments, each K₈₈ is, independently, an amino acid selected from the group consisting of R and S. In some embodiments, K₈₉ is an amino acid selected from the group consisting of K, R, H, G, E, T, Y, and I. In some embodiments, K₉₀ is an amino acid selected from the group consisting of R, S, G, N, Q, A, Y, and W. In some embodiments, K₉₀ is R. In some embodiments, K₉₁ is an amino acid selected from the group consisting of V, I, and F. In some embodiments, K₉₂ is an amino acid selected from the group consisting of A, G, P, M, N, V, and S. In some embodiments, K₉₂ is an amino acid selected from the group consisting of A, G, P, and M. In some embodiments, K₉₃ is an amino acid selected from the groups consisting of E, D, Q, S, R, K, M, and L.

### Variants of SEQ ID NO. 74 (Formula XIV)

In some embodiments, the pro-protein signal peptide comprises an amino acid sequence represented by:

(M₁)_{b}-(M₂)_{b}-(M₃)_{b}-(M₄)_{b}-(M₅)_{b}-(M₆)_{b}-(M₇)_{b}-(M₈)_{b}-(M₉)_{b}-(M₁₀)_{b}-(M₁₁)_{b}- (M₁₂)_{b}-(M₁₃)_{b}-(M₁₄)_{b}-(M₁₅)_{b}-(M₁₆)_{b}-(M₁₇)_{b}-(M₁₈)_{b}-(M₁₉)_{b}-(M₂₀)_{b}-(M₂₁)_{b}-(M₂₂)_{b}-(M₂₃)_{b}-(M₂₄)_{b}-(M₂₅)_{b}-(M₂₆)_{b}-(M₂₇)_{b}-(M₂₈)_{b}-(M₂₉)_{b}-(M₃₀)_{b}-(M₃₁)_{b}-(M₃₂)_{b}-(M₃₃)_{b}-(M₃₄)_{b}-(M₃₅)_{b}-(M₃₆)_{b}-(M₃₇)_{b}-(M₃₈)_{b}-(M₃₉)_{b}-(M₄₀)_{b}-(M₄₁)_{b}-(M₄₂)_{b}-(M₄₃)_{b}-(M₄₄)_{b}-(M₄₅)_{b}-(M₄₆)_{b}-(M₄₇)_{b}-(M₄₈)_{b}-(M₄₉)_{b}-(M₅₀)_{b}-(M₅₁)_{b}-(M₅₂)_{b}-(M₅₃)_{b}-(M₅₄)_{b}-(M₅₅)_{b}-(M₅₆)_{b}-(M₅₇)_{b}-(M₅₈)_{b}-(M₅₉)_{b}-(M₆₀)_{b}-(M₆₁)_{b}-(M₆₂)_{b}-(M₆₃)_{b}-(M₆₄)_{b}-(M₆₅)_{b}-(M₆₆)_{b}-(M₆₇)_{c}-(M₆₈)_{c}-(M₆₉)_{c}-(M₇₀)_{c} (Formula XIV)

wherein each b is, independently, 0, 1, 2, or 3, and each c is, independently, 1 or 2. Table 15 below describes the various amino acids that may be used at each position, with preferable amino acids underlined.

TABLE 15

| | Suitable | Isoelectric | Molecular | HP | |
|---|---|---|---|---|---|
| Position | Amino Acids | Point | Weight (g/mol) | Index | Helicity |
| M₁ | A, T, C, S, Y, E, H, V, W, I, | 2.7-10.8 | 75-205 | −5.1-34 | 0.5-1.3 |
| | L, F, G, Q, N, P, R, K, D, | | | | |
| | and M | | | | |
| M₂ | S, T, A, N, R, G, E, P, V, F, | 2.7-10.8 | 75-205 | −5.1-34 | 0.5-1.3 |
| | L, Q, K, H, D, I, C, Y, M, | | | | |
| | and W | | | | |
| M₃ | G, S, R, A, T, Q, E, D, C, | 2.7-10.8 | 75-182 | −3.7-25 | 0.7-1.3 |
| | Y, V, I, L, and N | | | | |
| M₄ | R, H, N, Q, E, A, Y, M, V, | 3.2-10.8 | 89-205 | −5.1-34 | 0.85-1.3 |
| | W, F, and L | | | | |
| M₅ | P, Y, A, T, Q, S, G, D, R, | 2.7-10.8 | 75-182 | −5.1-25 | 0.5-1.3 |
| | K, C, V, I, L, and H | | | | |
| M₆ | T, Q, N, S, A, E, G, D, H, | 2.7-10.8 | 75-205 | −5.1-34 | 0.5-1.3 |
| | P, F, L, C, K, V, R, Y, I, M, | | | | |
| | and W | | | | |
| M₇ | A, G, S, Q, N, K, D, T, C, | 2.7-10.8 | 75-205 | −5.1-34 | 0.5-1.3 |
| | Y, E, H, V, W, I, L, F, P, R, | | | | |
| | and M | | | | |
| M₈ | T, Q, N, S, A, G, C, R, K, | 2.7-10.8 | 75-205 | −5.1-34 | 0.5-1.3 |
| | P, Y, M, V, I, L, F, E, W, | | | | |
| | D, and H | | | | |
| M₉ | G, S, H, P, R, A, T, Q, E, D, | 2.7-10.8 | 75-205 | −5.1-34 | 0.5-1.3 |
| | C, Y, V, I, L, N, W, F, K, | | | | |
| | and M | | | | |
| M₁₀ | Q, E, and W | 3.2-5.8 | 146-205 | −0.5-34 | 0.85-1.07 |
| M₁₁ | V, I, L, F, C, A, and T | 5.05-6.05 | 89-165 | 2.8-31 | 0.75-1.3 |
| M₁₂ | S, G, A, N, Q, R, T, K, E, | 2.7-10.8 | 75-205 | −5.1-34 | 0.5-1.3 |
| | H, D, P, I, F, V, C, Y, L, M, | | | | |
| | and W | | | | |
| M₁₃ | T, Q, N, S, D, P, F, A, E, G, | 2.7-10.8 | 75-205 | −5.1-34 | 0.5-1.3 |
| | H, L, C, K, V, R, Y, I, M, | | | | |
| | and W | | | | |
| M₁₄ | L, F, I, V, M, Y, A, T, Q, N, | 2.7-10.8 | 75-182 | −5.1-31 | 0.5-1.3 |
| | S, D, K, P, E, R, H, G, and | | | | |
| | C | | | | |
| M₁₅ | S, P, V, E, T, A, F, L, N, R, | 2.7-10.8 | 75-205 | −5.1-34 | 0.5-1.3 |
| | G, Q, K, H, D, I, C, Y, M, | | | | |
| | and W | | | | |
| M₁₆ | T, S, A, E, G, C, R, P, Y, | 2.7-10.8 | 75-205 | −5.1-34 | 0.5-1.3 |
| | M, V, W, I, F, L, Q, N, D, | | | | |
| | H, and K | | | | |
| M₁₇ | D, E, Q, T, K, P, F, N, S, G, | 2.7-10.8 | 75-182 | −3.7-31 | 0.5-1.3 |
| | A, Y, R, and V | | | | |
| M₁₈ | G, S, H, P, R, D, N, A, T, | 2.7-10.8 | 75-205 | −5.1-34 | 0.5-1.3 |
| | Q, E, C, Y, V, I, L, W, F, K, | | | | |
| | and M | | | | |
| M₁₉ | T, P, F, S, A, E, G, C, R, Y, | 2.7-10.8 | 75-205 | −5.1-34 | 0.5-1.3 |
| | M, V, W, I, L, Q, N, D, H, | | | | |
| | and K | | | | |
| M₂₀ | L, F, I, V, Y, A, T, Q, S, D, | 2.7-10.8 | 75-182 | −5.1-31 | 0.5-1.3 |
| | M, N, K, P, E, R, H, G, and | | | | |
| | C | | | | |
| M₂₁ | F, L, W, Y, and P | 5.4-6.5 | 115-205 | 9.4-34 | 0.5-1.3 |
| M₂₂ | P, K, Y, A, T, Q, S, G, D, | 2.7-10.8 | 75-182 | −5.1-25 | 0.5-1.3 |
| | R, C, V, I, L, and H | | | | |
| M₂₃ | T, P, F, S, A, E, G, C, R, Y, | 2.7-10.8 | 75-205 | −5.1-34 | 0.5-1.3 |
| | M, V, W, I, L, Q, N, D, H, | | | | |
| | and K | | | | |
| M₂₄ | S, T, A, N, R, G, E, P, V, F, | 2.7-10.8 | 75-205 | −5.1-34 | 0.5-1.3 |
| | L, Q, K, H, D, I, C, Y, M, | | | | |
| | and W | | | | |
| M₂₅ | F, W, Y, and P | 5.4-6.5 | 115-205 | 9.4-34 | 0.5-1.3 |
| M₂₆ | T, P, F, Q, N, S, A, E, G, D, | 2.7-9.8 | 75-182 | −5.1-31 | 0.5-1.3 |
| | K, Y, C, V, I, L, and H | | | | |
| M₂₇ | D, E, Q, N, S, T, R, K, G, | 2.7-10.8 | 75-182 | −3.7-31 | 0.5-1.3 |
| | A, Y, P, V, and F | | | | |
| M₂₈ | T, Q, N, S, A, G, C, R, K, | 2.7-10.8 | 75-205 | −5.1-34 | 0.5-1.3 |
| | P, Y, M, V, I, L, F, E, W, | | | | |
| | D, and H | | | | |
| M₂₉ | S, T, E, A, P, V, F, L, N, R, | 2.7-10.8 | 75-205 | −5.1-34 | 0.5-1.3 |
| | G, Q, K, H, D, I, C, Y, M, | | | | |
| | and W | | | | |
| M₃₀ | D, Q, N, H, K, G, C, and Y | 2.7-9.8 | 75-182 | −5.1-16 | 0.75-1.2 |
| M₃₁ | F, L, W, Y, and P | 5.4-6.5 | 115-205 | 9.4-34 | 0.5-1.3 |
| M₃₂ | S, T, E, A, P, V, F, L, N, R, | 2.7-10.8 | 75-205 | −5.1-34 | 0.5-1.3 |
| | G, Q, K, H, D, I, C, Y, M, | | | | |
| | and W | | | | |
| M₃₃ | A, G, S, Q, N, K, D, T, C, | 2.7-10.8 | 75-205 | −5.1-34 | 0.5-1.3 |
| | Y, E, H, V, W, I, L, F, P, R, | | | | |
| | and M | | | | |
| M₃₄ | T, A, V, I, P, F, Q, N, S, E, | 2.7-9.8 | 75-182 | −5.1-31 | 0.5-1.3 |
| | G, D, K, Y, C, L, and H | | | | |
| M₃₅ | G, S, R, N, H, D, P, A, T, | 2.7-10.8 | 75-205 | −5.1-34 | 0.5-1.3 |
| | Q, E, C, Y, V, I, L, W, F, K, | | | | |
| | and M | | | | |
| M₃₆ | T, Q, S, A, E, D, K, H, P, | 2.7-9.8 | 75-205 | −5.1-34 | 0.5-1.3 |
| | Y, V, W, I, F, L, N, G, and | | | | |
| | C | | | | |
| M₃₇ | I, L, W, V, and M | 5.7-6.1 | 115-205 | 14-34 | 1.05-1.3 |
| M₃₈ | A, G, S, Q, N, K, D, C, P, | 2.7-10.8 | 75-205 | −5.1-34 | 0.5-1.3 |
| | R, Y, E, V, W, T, H, M, and | | | | |
| | F | | | | |
| M₃₉ | S, T, E, P, V, A, F, L, N, R, | 2.7-10.8 | 75-205 | −5.1-34 | 0.5-1.3 |
| | G, Q, K, H, D, I, C, Y, M, | | | | |
| | and W | | | | |
| M₄₀ | T, S, A, D, P, M, Q, E, K, | 2.7-9.8 | 75-205 | −5.1-34 | 0.5-1.3 |
| | H, Y, V, W, I, F, L, N, G, | | | | |
| | and C | | | | |
| M₄₁ | L, F, I, V, Y, A, T, Q, S, D, | 2.7-10.8 | 75-182 | −5.1-31 | 0.5-1.3 |
| | M, N, K, P, E, R, H, G, and | | | | |
| | C | | | | |
| M₄₂ | P, Y, A, T, Q, S, N, W, G, I, | 2.7-9.8 | 75-205 | −5.1-34 | 0.5-1.3 |
| | E, D, L, K, and H | | | | |
| M₄₃ | S, E, P, V, T, A, F, L, N, R, | 2.7-10.8 | 75-205 | −5.1-34 | 0.5-1.3 |
| | G, Q, K, H, D, I, C, Y, M, | | | | |
| | and W | | | | |
| M₄₄ | N, Q, S, E, D, T, H, K, G, | 2.7-9.8 | 75-205 | −5.1-34 | 0.5-1.3 |
| | A, P, W, and F | | | | |
| M₄₅ | V, I, L, F, C, A, and T | 5.0-6.1 | 89-166 | 2.5-31 | 0.7-1.3 |
| M₄₆ | A, T, S, N, R, Y, K, D, H, | 2.7-10.8 | 75-205 | −5.1-34 | 0.5-1.3 |
| | M, L, F, G, Q, C, P, E, V, | | | | |
| | and W | | | | |
| M₄₇ | I, L, and V | 5.9-6.1 | 115-132 | 14-25 | 1.25-1.3 |
| M₄₈ | S, P, V, E, T, A, F, L, N, R, | 2.7-10.8 | 75-205 | −5.1-34 | 0.5-1.3 |
| | G, Q, K, H, D, I, C, Y, M, | | | | |
| | and W | | | | |
| M₄₉ | F, V, A, T, Q, N, S, E, G, | 2.7-7.6 | 75-166 | −5.1-31 | 0.8-1.3 |
| | D, and H | | | | |
| M₅₀ | L, F, I, V, Y, A, T, Q, S, D, | 2.7-10.8 | 75-182 | −5.1-31 | 0.5-1.3 |
| | M, N, K, P, E, R, H, G, and | | | | |
| | C | | | | |
| M₅₁ | G, S, R, H, D, P, N, A, T, | 2.7-10.8 | 75-205 | −5.1-34 | 0.5-1.3 |
| | Q, E, C, Y, V, I, L, W, F, K, | | | | |
| | and M | | | | |
| M₅₂ | T, N, S, G, C, R, H, A, D, | 2.7-10.8 | 75-205 | −5.1-34 | 0.5-1.3 |
| | P, M, Q, E, K, Y, V, W, I, | | | | |
| | F, and L | | | | |
| M₅₃ | L, L, W, V, and M | 5.7-6.1 | 115-205 | 14-34 | 1.05-1.3 |
| M₅₄ | P, K, Y, A, T, Q, S, G, D, | 2.7-10.8 | 75-182 | −5.1-25 | 0.5-1.3 |
| | R, C, V, I, L, and H | | | | |
| M₅₅ | D, E, Q, N, S, K, G, A, Y, | 2.7-10.8 | 75-182 | −3.7-31 | 0.5-1.3 |
| | P, F, T, R, and V | | | | |
| M₅₆ | L, F, I, V, Y, P, A, T, Q, N, | 2.7-10.8 | 75-182 | −5.1-31 | 0.5-1.3 |
| | S, G, E, D, K, H, M, C, and | | | | |
| | R | | | | |
| M₅₇ | S, P, V, E, T, A, F, L, N, R, | 2.7-10.8 | 75-205 | −5.1-34 | 0.5-1.3 |
| | G, Q, K, H, D, I, C, Y, M, | | | | |
| | and W | | | | |
| M₅₈ | P, M, V, I, L, and F | 5.4-6.4 | 115-166 | 9.4-31 | 0.5-1.3 |
| M₅₉ | N, Q, S, E, D, T, R, K, G, | 2.7-10.8 | 75-182 | −3.7-16 | 0.8-1.3 |
| | A, and Y | | | | |
| M₆₀ | G, S, H, P, R, D, N, A, T, | 2.7-10.8 | 75-205 | −5.1-34 | 0.5-1.3 |
| | Q, E, C, Y, V, I, L, W, F, K, | | | | |
| | and M | | | | |
| M₆₁ | S, P, V, T, A, R, K, E, H, C, | 2.7-10.8 | 75-205 | −5.1-34 | 0.5-1.3 |
| | Y, I, F, L, N, Q, G, D, M, | | | | |
| | and W | | | | |
| M₆₂ | P, K, A, Y, T, Q, S, G, D, | 2.7-10.8 | 75-182 | −5.1-25 | 0.5-1.3 |
| | R, C, V, I, L, and H | | | | |
| M₆₃ | A, G, S, N, E, K, D, H, M, | 2.7-10.8 | 75-205 | −5.1-34 | 0.5-1.3 |
| | V, W, I, L, F, T, R, Y, Q, C, | | | | |
| | and P | | | | |
| M₆₄ | D, E, Q, T, K, P, F, N, S, G, | 2.7-10.8 | 75-182 | −3.7-31 | 0.5-1.3 |
| | A, Y, R, and V | | | | |
| M₆₅ | L, V, F, I, Y, P, A, T, Q, N, | 2.7-10.8 | 75-182 | −5.1-31 | 0.5-1.3 |
| | S, G, E, D, K, H, M, C, and | | | | |
| | R | | | | |
| M₆₆ | S, N, R, T, G, K, E, H, D, | 2.7-10.8 | 75-205 | −5.1-34 | 0.5-1.3 |
| | A, P, V, C, Y, I, F, L, Q, M, | | | | |
| | and W | | | | |
| M₆₇ | K, R, H, S, G, N, Q, D, E, | 2.7-10.8 | 75-205 | −5.1-34 | 0.5-1.3 |
| | T, A, C, P, Y, M, V, W, I, | | | | |
| | L, and F | | | | |
| M₆₈ | R, K, H, S, G, N, Q, D, E, | 2.7-10.8 | 75-205 | −5.1-34 | 0.5-1.3 |
| | T, A, C, P, Y, M, V, W, I, | | | | |
| | L, and F | | | | |
| M₆₉ | S, A, N, Q, R, T, G, K, E, | 2.7-10.8 | 75-205 | −5.1-34 | 0.5-1.3 |
| | H, D, A, C, P, Y, M, V, W, | | | | |
| | I, F, and L | | | | |
| M₇₀ | T, Q, N, S, A, E, G, D, C, | 2.7-10.8 | 75-205 | −5.1-34 | 0.5-1.3 |
| | R, K, H, P, Y, M, V, W, I, | | | | |
| | F, and L | | | | |

In some embodiments, amino acid positions M₁-M₆₆ may be omitted or repeated up to 2 extra time (i.e., be included 0 to 3 times), each repeat being independently selected from the indicated amino acids. It is to be understood that the omission or repetition of any amino acid positions M₁-M₆₆ is independent of the omission or repetition of any amino acid at an alternate position. In some embodiments, amino acid positions M₆₇-M₇₀ may be repeated up to 1 extra time (i.e., be included 1 to 2 times), each repeat being independently selected from the indicated amino acids. It is to be understood that the repetition of any amino acid positions M₆₇-M₇₀ is independent of the repetition of any amino acid at an alternate position.

In some embodiments, each M₁ is, independently, absent. In some embodiments, each M₁ is, independently, an amino acid selected from the group consisting of A, T, C, S, Y, E, H, V, W, I, L, F, G, Q, N, P, R, K, D, and M. In some embodiments, each M₁ is, independently, A. In some embodiments, each M₂ is, independently, absent. In some embodiments, each M₂ is, independently, an amino acid selected from the group consisting of S, T, A, N, R, G, E, P, V, F, L, Q, K, H, D, I, C, Y, M, and W. In some embodiments, each M₂ is, independently, S. In some embodiments, each M₃ is, independently, absent. In some embodiments, each M₃ is, independently, an amino acid selected from the group consisting of G, S, R, A, T, Q, E, D, C, Y, I, L, and N. In some embodiments, each M₃ is, independently, G. In some embodiments, each M₄ is, independently, absent. In some embodiments, each M₄ is, independently, an amino acid selected from the group consisting of R, H, N, Q, E, A, Y, M, V, W, F, and L. In some embodiments, each M₄ is, independently, R. In some embodiments, each M₅ is, independently, absent. In some embodiments, each M₅ is, independently, an amino acid selected from the group consisting of P, Y, A, T, Q, S, G, D, R, K, C, V, I, L, and H. In some embodiments, each M₅ is, independently, P. In some embodiments, each M₆ is, independently, absent. In some embodiments, each M₆ is, independently, an amino acid selected from the group consisting of T, Q, N, S, A, E, G, D, H, P, F, L, C, K, V, R, Y, I, M, and W. In some embodiments, each M₆ is, independently, T. In some embodiments, each M₇ is, independently, absent. In some embodiments, each M₇ is, independently, an amino acid selected from the group consisting of A, G, S, Q, N, K, D, T, C, Y, E, H, V, W, I, L, F, P, R, and M. In some embodiments, each M₇ is, independently, A. In some embodiments, each M₈ is, independently, absent. In some embodiments, each M₈ is, independently, an amino acid selected from the group consisting of T, Q, N, S, A, G, C, R, K, P, Y, M, V, I, L, F, E, W, D, and H. In some embodiments, each M₈ is, independently, T. In some embodiments, each M₉ is, independently, absent. In some embodiments, each M₉ is, independently, an amino acid selected from the group consisting of G, S, H, P, R, A, T, Q, E, D, C, Y, V, I, L, N, W, F, K, and M. In some embodiments, each M₉ is, independently, G. In some embodiments, each M₁₀ is, independently, absent. In some embodiments, each M₁₀ is, independently, an amino acid selected from the group consisting of Q, E, and W. In some embodiments, each Mn is, independently, absent. In some embodiments, each M₁₁ is, independently, an amino acid selected from the group consisting of V, I, L, F, C, A, and T. In some embodiments, each M₁₁ is, independently, an amino acid selected from the group consisting of V, I, and L. In some embodiments, each M₁₂ is, independently, absent. In some embodiments, each M₁₂ is, independently, an amino acid selected from the group consisting of S, G, A, N, Q, R, T, K, E, H, D, P, I, F, V, C, Y, L, M, and W. In some embodiments, each M₁₂ is, independently, S. In some embodiments, each M₁₃ is, independently, absent. In some embodiments, each M₁₃ is, independently, an amino acid selected from the group consisting of T, Q, N, S, D, P, F, A, E, G, H, L, C, K, V, R, Y, I, M, and W. In some embodiments, each M₁₃ is, independently, T. In some embodiments, each M₁₄ is, independently, absent. In some embodiments, each M₁₄ is, independently, an amino acid selected from the group consisting of L, F, I, V, M, Y, A, T, Q, N, S, D, K, P, E, R, H, G, and C. In some embodiments, each M₁₄ is, independently, L. In some embodiments, each M₁₅ is, independently, absent. In some embodiments, each M₁₅ is, independently, an amino acid selected from the group consisting of S, P, V, E, T, A, F, L, N, R, G, Q, K, H, D, I, C, Y, M, and W. In some embodiments, each M₁₅ is, independently, S. In some embodiments, each M₁₆ is, independently, absent. In some embodiments, each M₁₆ is, independently, an amino acid selected from the group consisting of T, S, A, E, G, C, R, P, Y, M, V, W, I, F, L, Q, N, D, H, and K. In some embodiments, each M₁₆ is, independently, T. In some embodiments, each M₁₇ is, independently, absent. In some embodiments, each M₁₇ is, independently, an amino acid selected from the group consisting of D, E, Q, T, K, P, F, N, S, G, A, Y, R, and V. In some embodiments, each M₁₇ is, independently, D. In some embodiments, each M₁₈ is, independently, absent. In some embodiments, each M₁₈ is, independently, an amino acid selected from the group consisting of G, S, H, P, R, D, N, A, T, Q, E, C, Y, V, I, L, W, F, K, and M. In some embodiments, each M₁₈ is, independently, G. In some embodiments, each M₁₉ is, independently, absent. In some embodiments, each M₁₉ is, independently, an amino acid selected from the group consisting of T, P, F, S, A, E, G, C, R, Y, M, V, W, I, L, Q, N, D, H, and K. In some embodiments, each M₁₉ is, independently, T. In some embodiments, each M₂₀ is, independently, absent. In some embodiments, each M₂₀ is, independently, an amino acid selected from the group consisting of L, F, I, V, Y, A, T, Q, S, D, M, N, K, P, E, R, H, G, and C. In some embodiments, each M₂₀ is, independently, L. In some embodiments, each M₂₁ is, independently, absent. In some embodiments, each M₂₁ is, independently, an amino acid selected from the group consisting of F, L, W, Y, and P. In some embodiments, each M₂₁ is, independently, F. In some embodiments, each M₂₂ is, independently, absent. In some embodiments, each M₂₂ is, independently, an amino acid selected from the group consisting of P, K, Y, A, T, Q, S, G, D, R, C, V, I, L, and H. In some embodiments, each M₂₂ is, independently, P. In some embodiments, each M₂₃ is, independently, absent. In some embodiments, each M₂₃ is, independently, an amino acid selected from the group consisting of T, P, F, S, A, E, G, C, R, Y, M, V, W, I, L, Q, N, D, H, and K. In some embodiments, each M₂₃ is, independently, T. In some embodiments, each M₂₄ is, independently, absent. In some embodiments, each M₂₄ is, independently, an amino acid selected from the group consisting of S, T, A, N, R, G, E, P, V, F, L, Q, K, H, D, I, C, Y, M, and W. In some embodiments, each M₂₄ is, independently, S. In some embodiments, each M₂₅ is, independently, absent. In some embodiments, each M₂₅ is, independently, an amino acid selected from the group consisting of F, W, Y, and P. In some embodiments, each M₂₅ is, independently, F. In some embodiments, each M₂₆ is, independently, absent. In some embodiments, each M₂₆ is, independently, an amino acid selected from the group consisting of T, P, F, Q, N, S, A, E, G, D, K, Y, C, V, I, L, and H. In some embodiments, each M₂₆ is, independently, T. In some embodiments, each M₂₇ is, independently, absent. In some embodiments, each M₂₇ is, independently, an amino acid selected from the group consisting of D, E, Q, N, S, T, R, K, G, A, Y, P, V, and F. In some embodiments, each M₂₇ is, independently, D. In some embodiments, each M₂₈ is, independently, absent. In some embodiments, each M₂₈ is, independently, an amino acid selected from the group consisting of T, Q, N, S, A, G, C, R, K, P, Y, M, V, I, L, F, E, W, D, and H. In some embodiments, each M₂₈ is, independently, T. In some embodiments, each M₂₉ is, independently, absent. In some embodiments, each M₂₉ is, independently, an amino acid selected from the group consisting of S, T, E, A, P, V, F, L, N, R, G, Q, K, H, D, I, C, Y, M, and W. In some embodiments, each M₂₉ is, independently, S. In some embodiments, each M₃₀ is, independently, absent. In some embodiments, each M₃₀ is, independently, an amino acid selected from the group consisting of D, Q, N, H, K, G, C, and Y. In some embodiments, each M₃₁ is, independently, absent. In some embodiments, each M₃₁ is, independently, an amino acid selected from the group consisting of F, L, W, Y, and P. In some embodiments, each M₃₁ is, independently, F. In some embodiments, each M₃₂ is, independently, absent. In some embodiments, each M₃₂ is, independently, an amino acid selected from the group consisting of S, T, E, A, P, V, F, L, N, R, G, Q, K, H, D, I, C, Y, M, and W. In some embodiments, each M₃₂ is, independently, S. In some embodiments, each M₃₃ is, independently, absent. In some embodiments, each M₃₃ is, independently, an amino acid selected from the group consisting of A, G, S, Q, N, K, D, T, C, Y, E, H, V, W, I, L, F, P, R, and M. In some embodiments, each M₃₃ is, independently, A. In some embodiments, each M₃₄ is, independently, absent. In some embodiments, each M₃₄ is, independently, an amino acid selected from the group consisting of T, A, V, I, P, F, Q, N, S, E, G, D, K, Y, C, L, and H. In some embodiments, each M₃₄ is, independently, T. In some embodiments, each M₃₅ is, independently, absent. In some embodiments, each M₃₅ is, independently, an amino acid selected from the group consisting of G, S, R, N, H, D, P, A, T, Q, E, C, Y, V, I, L, W, F, K, and M. In some embodiments, each M₃₅ is, independently, G. In some embodiments, each M₃₆ is, independently, absent. In some embodiments, each M₃₆ is, independently, an amino acid selected from the group consisting of T, Q, S, A, E, D, K, H, P, Y, V, W, I, F, L, N, G, and C. In some embodiments, each M₃₆ is, independently, T. In some embodiments, each M₃₇ is, independently, absent. In some embodiments, each M₃₇ is, independently, an amino acid selected from the group consisting of I, L, W, V, and M. In some embodiments, each M₃₇ is, independently, I. In some embodiments, each M₃₈ is, independently, absent. In some embodiments, each M₃₈ is, independently, an amino acid selected from the group consisting of A, G, S, Q, N, K, D, C, P, R, Y, E, V, W, T, H, M, and F. In some embodiments, each M₃₈ is, independently, A. In some embodiments, each M₃₉ is, independently, absent. In some embodiments, each M₃₉ is, independently, an amino acid selected from the group consisting of S, T, E, P, V, A, F, L, N, R, G, Q, K, H, D, I, C, Y, M, and W. In some embodiments, each M₃₉ is, independently, S. In some embodiments, each M₄₀ is, independently, absent. In some embodiments, each M₄₀ is, independently, an amino acid selected from the group consisting of T, S, A, D, P, M, Q, E, K, H, Y, V, W, I, F, L, N, G, and C. In some embodiments, each M₄₀ is, independently, T. In some embodiments, each M₄₁ is, independently, absent. In some embodiments, each M₄₁ is, independently, an amino acid selected from the group consisting of L, F, I, V, Y, A, T, Q, S, D, M, N, K, P, E, R, H, G, and C. In some embodiments, each M₄₁ is, independently, L. In some embodiments, each M₄₂ is, independently, absent. In some embodiments, each M₄₂ is, independently, an amino acid selected from the group consisting of P, Y, A, T, Q, S, N, W, G, I, E, D, L, K, and H. In some embodiments, each M₄₃ is, independently, absent. In some embodiments, each M₄₃ is, independently, an amino acid selected from the group consisting of S, E, P, V, T, A, F, L, N, R, G, Q, K, H, D, I, C, Y, M, and W. In some embodiments, each M₄₃ is, independently, S. In some embodiments, each M₄₄ is, independently, absent. In some embodiments, each M₄₄ is, independently, an amino acid selected from the group consisting of N, Q, S, E, D, T, H, K, G, A, P, W, and F. In some embodiments, each M₄₅ is, independently, absent. In some embodiments, each M₄₅ is, independently, an amino acid selected from the group consisting of V, I, L, F, C, A, and T. In some embodiments, each M₄₅ is, independently, an amino acid selected from the group consisting of V, I, and L. In some embodiments, each M₄₆ is, independently, absent. In some embodiments, each M₄₆ is, independently, an amino acid selected from the group consisting of A, T, S, N, R, Y, K, D, H, M, L, F, G, Q, C, P, E, V, and W. In some embodiments, each M₄₆ is, independently, A. In some embodiments, each M₄₇ is, independently, absent. In some embodiments, each M₄₇ is, independently, an amino acid selected from the group consisting of I, L, and V. In some embodiments, each M₄₇ is, independently, I. In some embodiments, each M₄₈ is, independently, absent. In some embodiments, each M₄₈ is, independently, an amino acid selected from the group consisting of S, P, V, E, T, A, F, L, N, R, G, Q, K, H, D, I, C, Y, M, and W. In some embodiments, each M₄₈ is, independently, S. In some embodiments, each M₄₉ is, independently, absent. In some embodiments, each M₄₉ is, independently, an amino acid selected from the group consisting of F, V, A, T, Q, N, S, E, G, D, and H. In some embodiments, each M₅₀ is, independently, absent. In some embodiments, each M₅₀ is, independently, an amino acid selected from the group consisting of L, F, I, V, Y, A, T, Q, S, D, M, N, K, P, E, R, H, G, and C. In some embodiments, each M₅₀ is, independently, L. In some embodiments, each M₅₁ is, independently, absent. In some embodiments, each M₅₁ is, independently, an amino acid selected from the group consisting of G, S, R, H, D, P, N, A, T, Q, E, C, Y, V, I, L, W, F, K, and M. In some embodiments, each M₅₁ is, independently, G. In some embodiments, each M₅₂ is, independently, absent. In some embodiments, each M₅₂ is, independently, an amino acid selected from the group consisting of T, N, S, G, C, R, H, A, D, P, M, Q, E, K, Y, V, W, I, F, and L. In some embodiments, each M₅₂ is, independently, T. In some embodiments, each M₅₃ is, independently, absent. In some embodiments, each M₅₃ is, independently, an amino acid selected from the group consisting of I, L, W, V, and M. In some embodiments, each M₅₃ is, independently, I. In some embodiments, each M₅₄ is, independently, absent. In some embodiments, each M₅₄ is, independently, an amino acid selected from the group consisting of P, K, Y, A, T, Q, S, G, D, R, C, V, I, L, and H. In some embodiments, each M₅₄ is, independently, P. In some embodiments, each M₅₅ is, independently, absent. In some embodiments, each M₅₅ is, independently, an amino acid selected from the group consisting of D, E, Q, N, S, K, G, A, Y, P, F, T, R, and V. In some embodiments, each M₅₅ is, independently, D. In some embodiments, each M₅₆ is, independently, absent. In some embodiments, each M₅₆ is, independently, an amino acid selected from the group consisting of L, F, I, V, Y, P, A, T, Q, N, S, G, E, D, K, H, M, C, and R. In some embodiments, each M₅₆ is, independently, L. In some embodiments, each M₅₇ is, independently, absent. In some embodiments, each M₅₇ is, independently, an amino acid selected from the group consisting of S, P, V, E, T, A, F, L, N, R, G, Q, K, H, D, I, C, Y, M, and W. In some embodiments, each M₅₇ is, independently, S. In some embodiments, each M₅₈ is, independently, absent. In some embodiments, each M₅₈ is, independently, an amino acid selected from the group consisting of P, M, V, I, L, and F. In some embodiments, each M₅₉ is, independently, absent. In some embodiments, each M₅₉ is, independently, an amino acid selected from the group consisting of N, Q, S, E, D, T, R, K, G, A, and Y. In some embodiments, each M₆₀ is, independently, absent. In some embodiments, each M₆₀ is, independently, an amino acid selected from the group consisting of G, S, H, P, R, D, N, A, T, Q, E, C, Y, V, I, L, W, F, K, and M. In some embodiments, each M₆₀ is, independently, G. In some embodiments, each M₆₁ is, independently, absent. In some embodiments, each M₆₁ is, independently, an amino acid selected from the group consisting of S, P, V, T, A, R, K, E, H, C, Y, I, F, L, N, Q, G, D, M, and W. In some embodiments, each M₆₁ is, independently, S. In some embodiments, each M₆₂ is, independently, absent. In some embodiments, each M₆₂ is, independently, an amino acid selected from the group consisting of P, K, A, Y, T, Q, S, G, D, R, C, V, I, L, and H. In some embodiments, each M₆₂ is, independently, P. In some embodiments, each M₆₃ is, independently, absent. In some embodiments, each M₆₃ is, independently, an amino acid selected from the group consisting of A, G, S, N, E, K, D, H, M, V, W, I, L, F, T, R, Y, Q, C, and P. In some embodiments, each M₆₃ is, independently, A. In some embodiments, each M₆₄ is, independently, absent. In some embodiments, each M₆₄ is, independently, an amino acid selected from the group consisting of D, E, Q, T, K, P, F, N, S, G, A, Y, R, and V. In some embodiments, each M₆₄ is, independently, D. In some embodiments, each M₆₅ is, independently, absent. In some embodiments, each M₆₅ is, independently, an amino acid selected from the group consisting of L, V, F, I, Y, P, A, T, Q, N, S, G, E, D, K, H, M, C, and R. In some embodiments, each M₆₅ is, independently, L. In some embodiments, each M₆₆ is, independently, absent. In some embodiments, each M₆₆ is, independently, an amino acid selected from the group consisting of S, N, R, T, G, K, E, H, D, A, P, V, C, Y, I, F, L, Q, M, and W. In some embodiments, each M₆₆ is, independently, S. In some embodiments, each M₆₇ is, independently, an amino acid selected from the group consisting of K, R, H, S, G, N, Q, D, E, T, A, C, P, Y, M, V, W, I, L, and F. In some embodiments, each M₆₇ is, independently, an amino acid selected from the group consisting of K, R, H, and S. In some embodiments, each M₆₈ is, independently, an amino acid selected from the group consisting of R, K, H, S, G, N, Q, D, E, T, A, C, P, Y, M, V, W, I, L, and F. In some embodiments, each M₆₈ is, independently, an amino acid selected from the group consisting of R, K, H, and S. In some embodiments, each M₆₉ is, independently, an amino acid selected from the group consisting of S, A, N, Q, R, T, G, K, E, H, D, A, C, P, Y, M, V, W, I, F, and L. In some embodiments, each M₆₉ is, independently, an amino acid selected from the group consisting of S, A, N, Q, R, and T. In some embodiments, each M₇₀ is, independently, an amino acid selected from the group consisting of T, Q, N, S, A, E, G, D, C, R, K, H, P, Y, M, V, W, I, F, and L. In some embodiments, each M₇₀ is, independently, an amino acid selected from the group consisting of T, Q, N, S, A, and E.

In some embodiments, the pro-protein signal peptide comprises an amino acid sequence of SEQ ID NO. 74.

### Variants of SEQ ID NO. 75 (Formula XV)

In some embodiments, the pro-protein signal peptide comprises an amino acid sequence represented by:

(N₁)_{b}-(N₂)_{b}-(N₃)_{b}-(N₄)_{b}-(N₅)_{b}-(N₆)_{b}-(N₇)_{b}-(N₈)_{b}-(N₉)_{b}-(N₁₀)_{b}-(N₁₁)_{b}- (N₁₂)_{b}-(N₁₃)_{b}-(N₁₄)_{b}-(N₁₅)_{b}-(N₁₆)_{b}-(N₁₇)_{b}-(N₁₈)_{b}-(N₁₉)_{b}-(N₂₀)_{b}-(N₂₁)_{b}-(N₂₂)_{b}-(N₂₃)_{b}-(N₂₄)_{b}-(N₂₅)_{b}-(N₂₆)_{b}-(N₂₇)_{b}-(N₂₈)_{b}-(N₂₉)_{b}-(N₃₀)_{b}-(N₃₁)_{b}-(N₃₂)_{b}-(N₃₃)_{b}-(N₃₄)_{b}-(N₃₅)_{b}-(N₃₆)_{b}-(N₃₇)_{b}-(N₃₈)_{b}-(N₃₉)_{b}-(N₄₀)_{b}-(N₄₁)_{b}-(N₄₂)_{b}-(N₄₃)_{b}-(N₄₄)_{b}-(N₄₅)_{b}-(N₄₆)_{b}-(N₄₇)_{b}-(N₄₈)_{b}-(N₄₉)_{b}-(N₅₀)_{b}-(N₅₁)_{b}-(N₅₂)_{b}-(N₅₃)_{b}-(N₅₄)_{b}-(N₅₅)_{b}-(N₅₆)_{b}-(N₅₇)_{b}-(N₅₈)_{b}-(N₅₉)_{b}-(N₆₀)_{b}-(N₆₁)_{b}-(N₆₂)_{b}-(N₆₃)_{b}-(N₆₄)_{b}-(N₆₅)_{b}-(N₆₆)_{b}-(N₆₇)_{c}-(N₆₈)_{c}-(N₆₉)_{c}-(N₇₀)_{c}-(N₇₁)_{c} (Formula XV)

wherein each b is, independently, 0, 1, 2, or 3, and each c is, independently, 1 or 2. Table 16 below describes the various amino acids that may be used at each position, with preferable amino acids underlined.

TABLE 16

| | Suitable | Isoelectric | Molecular | HP | |
|---|---|---|---|---|---|
| Position | Amino Acids | Point | Weight (g/mol) | Index | Helicity |
| N₁ | S, N, D, Q, R, T, G, E, | 2.7-10.8 | 75-205 | −5.1-34 | 0.5-1.3 |
| | H, A, P, M, V, K, Y, | | | | |
| | W, F, L, I, and C | | | | |
| N₂ | P, A, S, Y, V, T, G, I, | 3.2-6.4 | 75-182 | −0.5-23 | 0.5-1.3 |
| | E, and C | | | | |
| N₃ | T, S, G, D, C, A, L, N, | 2.7-10.8 | 75-205 | −3.7-34 | 0.5-1.3 |
| | R, P, Y, V, W, I, and | | | | |
| | F | | | | |
| N₄ | S, R, E, A, Q, K, N, | 2.7-10.8 | 75-205 | −5.1-34 | 0.5-1.3 |
| | D, T, G, H, C, P, Y, I, | | | | |
| | F, L, M, V, and W | | | | |
| N₅ | T, Q, N, G, C, M, S, | 2.7-6.05 | 75-205 | −1-34 | 0.75-1.3 |
| | A, E, D, Y, V, I, F, L, | | | | |
| | and W | | | | |
| N₆ | I, V, L, F, W, Y, A, T, | 2.7-7.6 | 89-205 | −5.1-34 | 0.8-1.3 |
| | S, E, D, and H | | | | |
| N₇ | P, V, A, S, N, G, E, L, | 3.2-9.8 | 75-148 | −3.7-25 | 0.5-1.3 |
| | and K | | | | |
| N₈ | A, G, Q, T, S, N, P, R, | 2.7-10.8 | 75-205 | −3.7-34 | 0.5-1.3 |
| | D, V, K, C, Y, W, I, | | | | |
| | L, and F | | | | |
| N₉ | F, Y, A, T, N, and R | 5.4-10.8 | 89-182 | −3.7-31 | 0.9-1.3 |
| N₁₀ | T, Q, N, R, K, M, S, | 2.7-10.8 | 105-205 | −5.1-34 | 0.5-1.3 |
| | E, D, H, P, V, W, I, F, | | | | |
| | and L | | | | |
| N₁₁ | A, G, Q, T, S, N, P, R, | 2.7-10.8 | 75-205 | −3.7-34 | 0.5-1.3 |
| | D, V, K, C, Y, W, I, | | | | |
| | L, and F | | | | |
| N₁₂ | S, N, Q, R, T, G, K, E, | 2.7-10.8 | 75-205 | −5.1-34 | 0.5-1.3 |
| | H, D, A, P, L, M, V, | | | | |
| | Y, W, F, I, and C | | | | |
| N₁₃ | L, F, I, W, V, M, Y, | 2.7-10.8 | 75-205 | −3.7-34 | 0.75-1.3 |
| | C, A, T, Q, N, S, G, E, | | | | |
| | D, and R | | | | |
| N₁₄ | V, I, L, A, T, S, G, R, | 2.7-10.8 | 75-182 | −5.1-31 | 0.5-1.3 |
| | P, Y, N, H, C, M, F, | | | | |
| | Q, E, K, and D | | | | |
| N₁₅ | S, N, Q, T, G, K, E, | 2.7-10.8 | 75-205 | −5.1-34 | 0.5-1.3 |
| | H, D, A, C, P, Y, I, F, | | | | |
| | L, R, M, V, and W | | | | |
| N₁₆ | T, N, S, A, D, R, P, Y, | 2.7-10.8 | 89-205 | −3.7-34 | 0.5-1.3 |
| | V, W, I, F, and L | | | | |
| N₁₇ | S, N, Q, R, K, E, D, | 2.7-10.8 | 75-205 | −5.1-34 | 0.5-1.3 |
| | A, T, G, H, C, P, Y, I, | | | | |
| | F, L, M, V, and W | | | | |
| N₁₈ | V, A, T, S, G, R, W, I, | 2.7-10.8 | 75-205 | −5.1-34 | 0.5-1.3 |
| | C, L, F, E, D, K, P, Y, | | | | |
| | N, H, M, and Q | | | | |
| N₁₉ | T, Q, N, S, A, E, G, | 2.7-6.05 | 75-205 | −1-34 | 0.8-1.3 |
| | D, Y, M, V, I, F, L, | | | | |
| | and W | | | | |
| N₂₀ | S, Q, R, K, E, A, N, | 2.7-10.8 | 75-205 | −5.1-34 | 0.5-1.3 |
| | D, T, G, H, C, P, Y, I, | | | | |
| | F, L, M, V, and W | | | | |
| N₂₁ | V, W, I, C, L, F, A, T, | 2.7-10.8 | 75-205 | −5.1-34 | 0.5-1.3 |
| | S, E, D, K, G, R, P, Y, | | | | |
| | N, H, M, and Q | | | | |
| N₂₂ | T, Q, N, S, A, D, C, | 2.7-10.8 | 75-205 | −5.1-34 | 0.5-1.3 |
| | K, P, Y, M, V, W, I, | | | | |
| | F, G, E, H, R, and L | | | | |
| N₂₃ | L, F, I, V, P, A, T, Q, | 2.7-10.8 | 75-182 | −5.1-31 | 0.5-1.3 |
| | S, G, R, K, H, M, Y, | | | | |
| | and D | | | | |
| N₂₄ | T, Q, S, A, G, P, Y, I, | 2.7-10.8 | 75-205 | −5.1-34 | 0.5-1.3 |
| | K, H, V, F, L, N, D, | | | | |
| | C, M, W, E, and R | | | | |
| N₂₅ | S, R, E, A, Q, K, N, | 2.7-10.8 | 75-205 | −5.1-34 | 0.5-1.3 |
| | D, T, G, H, C, P, Y, I, | | | | |
| | F, L, M, V, and W | | | | |
| N₂₆ | T, N, D, S, A, R, P, Y, | 2.7-10.8 | 89-205 | −3.7-34 | 0.5-1.3 |
| | V, W, I, F, and L | | | | |
| N₂₇ | D, N, R, E, Q, S, H, T, | 2.7-10.8 | 75-205 | −5.1-34 | 0.5-1.3 |
| | K, G, W, I, P, and Y | | | | |
| N₂₈ | V, A, T, S, G, R, W, I, | 2.7-10.8 | 75-205 | −5.1-34 | 0.5-1.3 |
| | C, L, F, E, D, K, P, Y, | | | | |
| | N, H, M, and Q | | | | |
| N₂₉ | T, S, A, D, C, L, N, R, | 2.7-10.8 | 89-205 | −3.7-34 | 0.5-1.3 |
| | P, Y, V, W, I, and F | | | | |
| N₃₀ | P, Y, V, A, T, S, G, I, | 3.2-6.4 | 75-182 | −0.5-23 | 0.5-1.3 |
| | E, and C | | | | |
| N₃₁ | T, Q, S, A, G, K, H, P, | 2.7-10.8 | 75-205 | −5.1-34 | 0.5-1.3 |
| | Y, V, I, F, L, N, D, C, | | | | |
| | M, W, E, and R | | | | |
| N₃₂ | S, R, E, A, Q, K, N, | 2.7-10.8 | 75-205 | −5.1-34 | 0.5-1.3 |
| | D, T, G, H, C, P, Y, I, | | | | |
| | F, L, M, V, and W | | | | |
| N₃₃ | E, D, Q, N, S, T, H, R, | 2.7-10.8 | 75-175 | −5.1-31 | 0.5-1.3 |
| | G, A, P, F, and L | | | | |
| N₃₄ | D, N, R, E, Q, S, H, T, | 2.7-10.8 | 75-205 | −5.1-34 | 0.5-1.3 |
| | K, G, W, I, P, and Y | | | | |
| N₃₅ | T, Q, S, A, G, P, Y, I, | 2.7-10.8 | 75-205 | −5.1-34 | 0.5-1.3 |
| | K, H, V, F, L, N, D, | | | | |
| | C, M, W, E, and R | | | | |
| N₃₆ | G, S, K, A, T, Q, D, | 2.7-9.8 | 75-205 | −3.7-34 | 0.5-1.3 |
| | C, P, Y, V, W, I, L, | | | | |
| | and F | | | | |
| N₃₇ | F, Y, A, T, N, and R | 5.4-10.8 | 89-182 | −3.7-31 | 0.9-1.3 |
| N₃₈ | V, A, T, S, G, R, W, I, | 2.7-10.8 | 75-205 | −5.1-34 | 0.5-1.3 |
| | C, L, F, E, D, K, P, Y, | | | | |
| | N, H, M and Q | | | | |
| N₃₉ | L, F, I, W, V, M, C, | 2.7-10.8 | 75-205 | −5.1-34 | 0.75-1.3 |
| | A, T, Q, N, S, G, D, | | | | |
| | R, K, and H | | | | |
| N₄₀ | P, A, S, Y, V, T, G, I, | 3.2-6.4 | 75-182 | −0.5-23 | 0.5-1.3 |
| | E, and C | | | | |
| N₄₁ | D, N, R, G, Y, E, Q, | 2.7-10.8 | 75-205 | −5.1-34 | 0.5-1.3 |
| | S, H, T, K, W, and I | | | | |
| N₄₂ | S, R, E, A, N, T, G, P, | 2.7-10.8 | 75-205 | −5.1-34 | 0.5-1.3 |
| | V, Q, K, H, D, Y, M, | | | | |
| | I, F, L, C, and W | | | | |
| N₄₃ | G, S, R, K, A, N, Q, | 2.7-10.8 | 75-205 | −5.1-34 | 0.5-1.3 |
| | H, E, D, P, W, L, and | | | | |
| | F | | | | |
| N₄₄ | T, Q, S, A, G, P, Y, I, | 2.7-10.8 | 75-205 | −5.1-34 | 0.5-1.3 |
| | N, E, D, C, K, H, R, | | | | |
| | V, L, M, F, and W | | | | |
| N₄₅ | S, T, G, A, V, I, R, E, | 2.7-10.8 | 75-205 | −5.1-34 | 0.5-1.3 |
| | N, P, Q, K, H, D, Y, | | | | |
| | M, F, L, C, and W | | | | |
| N₄₆ | C | — | — | — | — |
| N₄₇ | S, N, R, T, G, K, E, H, | 2.7-10.8 | 75-205 | −5.1-34 | 0.5-1.3 |
| | D, A, P, Y, V, W, I, L, | | | | |
| | Q, M, F, and C | | | | |
| N₄₈ | G, S, R, K, N, T, Q, | 2.7-10.8 | 75-175 | −5.1-25 | 0.5-1.3 |
| | H, E, D, P, I, and L | | | | |
| N₄₉ | T, S, G, D, C, A, L, N, | 2.7-10.8 | 75-205 | −3.7-34 | 0.5-1.3 |
| | R, P, Y, V, W, I, and | | | | |
| | F | | | | |
| N₅₀ | V, A, T, S, G, I, R, P, | 3.2-10.8 | 75-182 | −5.1-31 | 0.5-1.3 |
| | Y, L, N, H, C, M, F, | | | | |
| | Q, E, and K | | | | |
| N₅₁ | A, T, G, S, Q, N, R, | 3.2-10.8 | 75-205 | −5.1-34 | 0.8-1.3 |
| | Y, E, H, M, V, W, I, | | | | |
| | L, and F | | | | |
| N₅₂ | D, E, Q, N, S, T, K, | 2.7-9.8 | 89-205 | −3.7-34 | 0.5-1.3 |
| | A, Y, P, M, W, I, F, | | | | |
| | and L | | | | |
| N₅₃ | A, T, C, G, S, N, P, R, | 2.7-10.8 | 75-175 | −5.1-31 | 0.5-1.3 |
| | K, D, H, M, and F | | | | |
| N₅₄ | L, F, I, V, P, A, T, Q, | 2.7-10.8 | 75-182 | −5.1-31 | 0.5-1.3 |
| | S, G, R, K, H, M, Y, | | | | |
| | and D | | | | |
| N₅₅ | E, D, N, T, R, K, G, | 2.7-10.8 | 75-175 | −3.7-15 | 0.8-1.3 |
| | A, and V | | | | |
| N₅₆ | A, G, Q, T, S, N, P, R, | 2.7-10.8 | 75-205 | −3.7-34 | 0.5-1.3 |
| | D, V, W, K, C, Y, I, | | | | |
| | L, and F | | | | |
| N₅₇ | Y, C, N, I, F, and L | 5.0-6.1 | 121-182 | 0-31 | 0.75-1.3 |
| N₅₈ | S, T, G, H, A, P, Y, V, | 2.7-10.8 | 75-205 | −5.1-34 | 0.5-1.3 |
| | F, L, N, R, K, E, D, | | | | |
| | W, I, Q, M, and C | | | | |
| N₅₉ | I, V, and L | 5.9-6.1 | 117-132 | 14-25 | 1.25-1.3 |
| N₆₀ | S | — | — | — | — |
| N₆₁ | G, S, R, K, A, N, T, | 2.7-10.8 | 75-182 | −3.7-16 | 0.5-1.3 |
| | Q, E, D, P, and Y | | | | |
| N₆₂ | I, V, L, F, W, Y, A, T, | 2.7-7.6 | 89-205 | −5.1-34 | 0.8-1.3 |
| | S, E, D, and H | | | | |
| N₆₃ | T, Q, N, G, C, M, S, | 2.7-6.1 | 75-205 | −1-34 | 0.75-1.3 |
| | A, E, D, Y, V, I, F, L, | | | | |
| | and W | | | | |
| N₆₄ | S, N, Q, R, G, K, E, | 2.7-10.8 | 75-205 | −5.1-34 | 0.5-1.3 |
| | D, P, Y, W, F, T, H, | | | | |
| | A, V, L, I, M, and C | | | | |
| N₆₅ | A, C, G, S, Q, N, R, | 2.7-10.8 | 75-182 | −5.1-25 | 0.75-1.3 |
| | Y, E, K, D, H, M, V, | | | | |
| | I, and L | | | | |
| N₆₆ | V, I, A, T, S, G, R, P, | 2.7-10.8 | 75-182 | −5.1-31 | 0.5-1.3 |
| | Y, L, N, H, C, M, F, | | | | |
| | Q, E, K, and D | | | | |
| N₆₇ | S, N, Q, R, T, G, K, E, | 2.7-10.8 | 75-205 | −5.1-34 | 0.5-1.3 |
| | H, D, A, C, P, Y, M, | | | | |
| | V, W, I, F, and L | | | | |
| N₆₈ | K, R, H, S, G, N, Q, | 2.7-10.8 | 75-205 | −5.1-34 | 0.5-1.3 |
| | D, E, T, A, C, P, Y, | | | | |
| | M, V, W, I, L, and F | | | | |
| N₆₉ | K, R, H, S, G, N, Q, | 2.7-10.8 | 75-205 | −5.1-34 | 0.5-1.3 |
| | D, E, T, A, C, P, Y, | | | | |
| | M, V, W, I, L, and F | | | | |
| N₇₀ | D, E, Q, N, S, H, T, R, | 2.7-10.8 | 75-205 | −5.1-34 | 0.5-1.3 |
| | K, G, A, C, Y, P, M, | | | | |
| | V, W, I, F, and L | | | | |
| N₇₁ | A, T, C, G, S, Q, N, P, | 2.7-10.8 | 75-205 | −5.1-34 | 0.5-1.3 |
| | R, Y, E, K, D, H, M, | | | | |
| | V, W, I, L, and F | | | | |

In some embodiments, amino acid positions N₁-N₆₆ may be omitted or repeated up to 2 extra time (i.e., be included 0 to 3 times), each repeat being independently selected from the indicated amino acids. It is to be understood that the omission or repetition of any amino acid positions N₁-N₆₆ is independent of the omission or repetition of any amino acid at an alternate position. In some embodiments, amino acid positions N₆₇-N₇₁ may be repeated up to 1 extra time (i.e., be included 1 to 2 times), each repeat being independently selected from the indicated amino acids. It is to be understood that the repetition of any amino acid positions N₆₇-N₇₁ is independent of the repetition of any amino acid at an alternate position.

In some embodiments, each N₁ is, independently, absent. In some embodiments, each N₁ is, independently, an amino acid selected from the group consisting of S, N, D, Q, R, T, G, E, H, A, P, M, V, K, Y, W, F, L, I, and C. In some embodiments, each N₁ is, independently, S. In some embodiments, each N₂ is, independently, absent. In some embodiments, each N₂ is, independently, an amino acid selected from the group consisting of P, A, S, Y, V, T, G, I, E, and C. In some embodiments, each N₂ is, independently, P. In some embodiments, each N₃ is, independently, absent. In some embodiments, each N₃ is, independently, an amino acid selected from the group consisting of T, S, G, D, C, A, L, N, R, P, Y, V, W, I, and F. In some embodiments, each N₃ is, independently, T. In some embodiments, each N₄ is, independently, absent. In some embodiments, each N₄ is, independently, an amino acid selected from the group consisting of S, R, E, A, Q, K, N, D, T, G, H, C, P, Y, I, F, L, M, V, and W. In some embodiments, each N₄ is, independently, S. In some embodiments, each N₅ is, independently, absent. In some embodiments, each N₅ is, independently, an amino acid selected from the group consisting of T, Q, N, G, C, M, S, A, E, D, Y, V, I, F, L, and W. In some embodiments, each N₅ is, independently, T. In some embodiments, each N₆ is, independently, absent. In some embodiments, each N₆ is, independently, an amino acid selected from the group consisting of I, V, L, F, W, Y, A, T, S, E, D, and H. In some embodiments, each N₆ is, independently, an amino acid selected from the group consisting of I and V. In some embodiments, each N₇ is, independently, absent. In some embodiments, each N₇ is, independently, an amino acid selected from the group consisting of P, V, A, S, N, G, E, L, and K. In some embodiments, each N₈ is, independently, absent. In some embodiments, each N₈ is, independently, an amino acid selected from the group consisting of A, G, Q, T, S, N, P, R, D, V, K, C, Y, W, I, L, and F. In some embodiments, each N₈ is, independently, an amino acid selected from the group consisting of A, G, and Q. In some embodiments, each N₉ is, independently, absent. In some embodiments, each N₉ is, independently, an amino acid selected from the group consisting of F, Y, A, T, N, and R. In some embodiments, each N₉ is, independently, an amino acid selected from the group consisting of F and Y. In some embodiments, each N₁₀ is, independently, absent. In some embodiments, each N₁₀ is, independently, an amino acid selected from the group consisting of T, Q, N, R, K, M, S, E, D, H, P, V, W, I, F, and L. In some embodiments, each N₁₀ is, independently, T. In some embodiments, each N₁₁ is, independently, absent. In some embodiments, each N₁₁ is, independently, an amino acid selected from the group consisting of A, G, Q, T, S, N, P, R, D, V, K, C, Y, W, I, L, and F. In some embodiments, each N₁₁ is, independently, an amino acid selected from the group consisting of A, G, and Q. In some embodiments, each N₁₂ is, independently, absent. In some embodiments, each N₁₂ is, independently, an amino acid selected from the group consisting of S, N, Q, R, T, G, K, E, H, D, A, P, L, M, V, Y, W, F, I, and C. In some embodiments, each N₁₂ is, independently, S. In some embodiments, each N₁₃ is, independently, absent. In some embodiments, each N₁₃ is, independently, an amino acid selected from the group consisting of L, F, I, W, V, M, Y, C, A, T, Q, N, S, G, E, D, and R. In some embodiments, each N₁₄ is, independently, absent. In some embodiments, each N₁₄ is, independently, an amino acid selected from the group consisting of V, I, L, A, T, S, G, R, P, Y, N, H, C, M, F, Q, E, K, and D. In some embodiments, each N₁₄ is, independently, V. In some embodiments, each N₁₅ is, independently, absent. In some embodiments, each N₁₅ is, independently, an amino acid selected from the group consisting of S, N, Q, T, G, K, E, H, D, A, C, P, Y, I, F, L, R, M, V, and W. In some embodiments, each N₁₅ is, independently, S. In some embodiments, each N₁₆ is, independently, absent. In some embodiments, each N₁₆ is, independently, an amino acid selected from the group consisting of T, N, S, A, D, R, P, Y, V, W, I, F, and L. In some embodiments, each N₁₆ is, independently, T. In some embodiments, each N₁₇ is, independently, absent. In some embodiments, each N₁₇ is, independently, an amino acid selected from the group consisting of S, N, Q, R, K, E, D, A, T, G, H, C, P, Y, I, F, L, M, V, and W. In some embodiments, each N₁₇ is, independently, S. In some embodiments, each N₁₈ is, independently, absent. In some embodiments, each N₁₈ is, independently, an amino acid selected from the group consisting of V, A, T, S, G, R, W, I, C, L, F, E, D, K, P, Y, N, H, M, and Q. In some embodiments, each N₁₈ is, independently, V. In some embodiments, each N₁₉ is, independently, absent. In some embodiments, each N₁₉ is, independently, an amino acid selected from the group consisting of T, Q, N, S, A, E, G, D, Y, M, V, I, F, L, and W. In some embodiments, each N₁₉ is, independently, T. In some embodiments, each N₂₀ is, independently, absent. In some embodiments, each N₂₀ is, independently, an amino acid selected from the group consisting of S, Q, R, K, E, A, N, D, T, G, H, C, P, Y, I, F, L, M, V, and W. In some embodiments, each N₂₀ is, independently, S. In some embodiments, each N₂₁ is, independently, absent. In some embodiments, each N₂₁ is, independently, an amino acid selected from the group consisting of V, W, I, C, L, F, A, T, S, E, D, K, G, R, P, Y, N, H, M, and Q. In some embodiments, each N₂₁ is, independently, V. In some embodiments, each N₂₂ is, independently, absent. In some embodiments, each N₂₂ is, independently, an amino acid selected from the group consisting of T, Q, N, S, A, D, C, K, P, Y, M, V, W, I, F, G, E, H, R, and L. In some embodiments, each N₂₂ is, independently, T. In some embodiments, each N₂₃ is, independently, absent. In some embodiments, each N₂₃ is, independently, an amino acid selected from the group consisting of L, F, I, V, P, A, T, Q, S, G, R, K, H, M, Y, and D. In some embodiments, each N₂₃ is, independently, an amino acid selected from the group consisting of L, F, I, V, P, A, T, Q, S, G, R, K, and H. In some embodiments, each N₂₄ is, independently, absent. In some embodiments, each N₂₄ is, independently, an amino acid selected from the group consisting of T, Q, S, A, G, P, Y, I, K, H, V, F, L, N, D, C, M, W, E, and R. In some embodiments, each N₂₄ is, independently, T. In some embodiments, each N₂₅ is, independently, absent. In some embodiments, each N₂₅ is, independently, an amino acid selected from the group consisting of S, R, E, A, Q, K, N, D, T, G, H, C, P, Y, I, F, L, M, V, and W. In some embodiments, each N₂₅ is, independently, S. In some embodiments, each N₂₆ is, independently, absent. In some embodiments, each N₂₆ is, independently, an amino acid selected from the group consisting of T, N, D, S, A, R, P, Y, V, W, I, F, and L. In some embodiments, each N₂₆ is, independently, T. In some embodiments, each N₂₇ is, independently, absent. In some embodiments, each N₂₇ is, independently, an amino acid selected from the group consisting of D, N, R, E, Q, S, H, T, K, G, W, I, P, and Y. In some embodiments, each N₂₇ is, independently, an amino acid selected from the group consisting of D and N. In some embodiments, each N₂₈ is, independently, absent. In some embodiments, each N₂₈ is, independently, an amino acid selected from the group consisting of V, A, T, S, G, R, W, I, C, L, F, E, D, K, P, Y, N, H, M, and Q. In some embodiments, each N₂₈ is, independently, V. In some embodiments, each N₂₉ is, independently, absent. In some embodiments, each N₂₉ is, independently, an amino acid selected from the group consisting of T, S, A, D, C, L, N, R, P, Y, V, W, I, and F. In some embodiments, each N₂₉ is, independently, T. In some embodiments, each N₃₀ is, independently, absent. In some embodiments, each N₃₀ is, independently, an amino acid selected from the group consisting of P, Y, V, A, T, S, G, I, E, and C. In some embodiments, each N₃₀ is, independently, P. In some embodiments, each N₃₁ is, independently, absent. In some embodiments, each N₃₁ is, independently, an amino acid selected from the group consisting of T, Q, S, A, G, K, H, P, Y, V, I, F, L, N, D, C, M, W, E, and R. In some embodiments, each N₃₁ is, independently, T. In some embodiments, each N₃₂ is, independently, absent. In some embodiments, each N₃₂ is, independently, an amino acid selected from the group consisting of S, R, E, A, Q, K, N, D, T, G, H, C, P, Y, I, F, L, M, V, and W. In some embodiments, each N₃₂ is, independently, S. In some embodiments, each N₃₃ is, independently, absent. In some embodiments, each N₃₃ is, independently, an amino acid selected from the group consisting of E, D, Q, N, S, T, H, R, G, A, P, F, and L. In some embodiments, each N₃₄ is, independently, absent. In some embodiments, each N₃₄ is, independently, an amino acid selected from the group consisting of D, N, R, E, Q, S, H, T, K, G, W, I, P, and Y. In some embodiments, each N₃₄ is, independently, an amino acid selected form the group consisting of D and N. In some embodiments, each N₃₅ is, independently, absent. In some embodiments, each N₃₅ is, independently, an amino acid selected from the group consisting of T, Q, S, A, G, P, Y, I, K, H, V, F, L, N, D, C, M, W, E, and R. In some embodiments, each N₃₅ is, independently, T. In some embodiments, each N₃₆ is, independently, absent. In some embodiments, each N₃₆ is, independently, an amino acid selected from the group consisting of G, S, K, A, T, Q, D, C, P, Y, V, W, I, L, and F. In some embodiments, each N₃₇ is, independently, absent. In some embodiments, each N₃₇ is, independently, an amino acid selected from the group consisting of F, Y, A, T, N, and R. In some embodiments, each N₃₇ is, independently, an amino acid selected from the group consisting of F and Y. In some embodiments, each N₃₈ is, independently, absent. In some embodiments, each N₃₈ is, independently, an amino acid selected from the group consisting of V, A, T, S, G, R, W, I, C, L, F, E, D, K, P, Y, N, H, M and Q. In some embodiments, each N₃₈ is, independently, V. In some embodiments, each N₃₉ is, independently, absent. In some embodiments, each N₃₉ is, independently, an amino acid selected from the group consisting of L, F, I, W, V, M, C, A, T, Q, N, S, G, D, R, K, and H. In some embodiments, each N₄₀ is, independently, absent. In some embodiments, each N₄₀ is, independently, an amino acid selected from the group consisting of P, A, S, Y, V, T, G, I, E, and C. In some embodiments, each N₄₀ is, independently, P. In some embodiments, each N₄₁ is, independently, absent. In some embodiments, each N₄₁ is, independently, an amino acid selected from the group consisting of D, N, R, G, Y, E, Q, S, H, T, K, W, and I. In some embodiments, each N₄₁ is, independently, an amino acid selected from the group consisting of D and N. In some embodiments, each N₄₂ is, independently, absent. In some embodiments, each N₄₂ is, independently, an amino acid selected from the group consisting of S, R, E, A, N, T, G, P, V, Q, K, H, D, Y, M, I, F, L, C, and W. In some embodiments, each N₄₂ is, independently, S. In some embodiments, each N₄₃ is, independently, absent. In some embodiments, each N₄₃ is, independently, an amino acid selected from the group consisting of G, S, R, K, A, N, Q, H, E, D, P, W, L, and F. In some embodiments, each N₄₄ is, independently, absent. In some embodiments, each N₄₄ is, independently, an amino acid selected from the group consisting of T, Q, S, A, G, P, Y, I, N, E, D, C, K, H, R, V, L, M, F, and W. In some embodiments, each N₄₄ is, independently, T. In some embodiments, each N₄₅ is, independently, absent. In some embodiments, each N₄₅ is, independently, an amino acid selected from the group consisting of S, T, G, A, V, I, R, E, N, P, Q, K, H, D, Y, M, F, L, C, and W. In some embodiments, each N₄₅ is, independently, S. In some embodiments, each N₄₆ is, independently, absent. In some embodiments, each N₄₆ is, independently, C. In some embodiments, each N₄₇ is, independently, absent. In some embodiments, each N₄₇ is, independently, an amino acid selected from the group consisting of S, N, R, T, G, K, E, H, D, A, P, Y, V, W, I, L, Q, M, F, and C. In some embodiments, each N₄₇ is, independently, S. In some embodiments, each N₄₈ is, independently, absent. In some embodiments, each N₄₈ is, independently, an amino acid selected from the group consisting of G, S, R, K, N, T, Q, H, E, D, P, I, and L. In some embodiments, each N₄₉ is, independently, absent. In some embodiments, each N₄₉ is, independently, an amino acid selected from the group consisting of T, S, G, D, C, A, L, N, R, P, Y, V, W, I, and F. In some embodiments, each N₄₉ is, independently, T. In some embodiments, each N₅₀ is, independently, absent. In some embodiments, each N₅₀ is, independently, an amino acid selected from the group consisting of V, A, T, S, G, I, R, P, Y, L, N, H, C, M, F, Q, E, and K. In some embodiments, each N₅₀ is, independently, V. In some embodiments, each N₅₁ is, independently, absent. In some embodiments, each N₅₁ is, independently, an amino acid selected from the group consisting of A, T, G, S, Q, N, R, Y, E, H, M, V, W, I, L, and F. In some embodiments, each N₅₂ is, independently, absent. In some embodiments, each N₅₂ is, independently, an amino acid selected from the group consisting of D, E, Q, N, S, T, K, A, Y, P, M, W, I, F, and L. In some embodiments, each N₅₃ is, independently, absent. In some embodiments, each N₅₃ is, independently, an amino acid selected from the group consisting of A, T, C, G, S, N, P, R, K, D, H, M, and F. In some embodiments, each N₅₄ is, independently, absent. In some embodiments, each N₅₄ is, independently, an amino acid selected from the group consisting of L, F, I, V, P, A, T, Q, S, G, R, K, H, M, Y, and D. In some embodiments, each N₅₄ is, independently, an amino acid selected from the group consisting of L, F, I, V, P, A, T, Q, S, G, R, K, and H. In some embodiments, each N₅₅ is, independently, absent. In some embodiments, each N₅₅ is, independently, an amino acid selected from the group consisting of E, D, N, T, R, K, G, A, and V. In some embodiments, each N₅₆ is, independently, absent. In some embodiments, each N₅₆ is, independently, an amino acid selected from the group consisting of A, G, Q, T, S, N, P, R, D, V, W, K, C, Y, I, L, and F. In some embodiments, each N₅₆ is, independently, an amino acid selected from the group consisting of A, G, and Q. In some embodiments, each N₅₇ is, independently, absent. In some embodiments, each N₅₇ is, independently, an amino acid selected from the group consisting of Y, C, N, I, F, and L. In some embodiments, each N₅₈ is, independently, absent. In some embodiments, each N₅₈ is, independently, an amino acid selected from the group consisting of S, T, G, H, A, P, Y, V, F, L, N, R, K, E, D, W, I, Q, M, and C. In some embodiments, each N₅₈ is, independently, S. In some embodiments, each N₅₉ is, independently, absent. In some embodiments, each N₅₉ is, independently, an amino acid selected from the group consisting of I, V, and L. In some embodiments, each N₅₉ is, independently, an amino acid selected from the group consisting of I and V. In some embodiments, each N₆₀ is, independently, absent. In some embodiments, each N₆₀ is, independently, S. In some embodiments, each N₆₁ is, independently, absent. In some embodiments, each N₆₁ is, independently, an amino acid selected from the group consisting of G, S, R, K, A, N, T, Q, E, D, P, and Y. In some embodiments, each N₆₂ is, independently, absent. In some embodiments, each N₆₂ is, independently, an amino acid selected from the group consisting of I, V, L, F, W, Y, A, T, S, E, D, and H. In some embodiments, each N₆₂ is, independently, an amino acid selected from the group consisting of I and V. In some embodiments, each N₆₃ is, independently, absent. In some embodiments, each N₆₃ is, independently, an amino acid selected from the group consisting of T, Q, N, G, C, M, S, A, E, D, Y, V, I, F, L, and W. In some embodiments, each N₆₃ is, independently, T. In some embodiments, each N₆₄ is, independently, absent. In some embodiments, each N₆₄ is, independently, an amino acid selected from the group consisting of S, N, Q, R, G, K, E, D, P, Y, W, F, T, H, A, V, L, I, M, and C. In some embodiments, each N₆₄ is, independently, S. In some embodiments, each N₆₅ is, independently, absent. In some embodiments, each N₆₅ is, independently, an amino acid selected from the group consisting of A, C, G, S, Q, N, R, Y, E, K, D, H, M, V, I, and L. In some embodiments, each N₆₆ is, independently, absent. In some embodiments, each N₆₆ is, independently, an amino acid selected from the group consisting of V, I, A, T, S, G, R, P, Y, L, N, H, C, M, F, Q, E, K, and D. In some embodiments, each N₆₆ is, independently, V. In some embodiments, each N₆₇ is, independently, an amino acid selected from the group consisting of S, N, Q, R, T, G, K, E, H, D, A, C, P, Y, M, V, W, I, F, and L. In some embodiments, each N₆₇ is, independently, an amino acid selected from the group consisting of S, N, Q, R, and T. In some embodiments, each N₆₈ is, independently, an amino acid selected from the group consisting of K, R, H, S, G, N, Q, D, E, T, A, C, P, Y, M, V, W, I, L, and F. In some embodiments, each N₆₈ is, independently, an amino acid selected from the group consisting of K, R, H, and S. In some embodiments, each N₆₉ is, independently, an amino acid selected from the group consisting of K, R, H, S, G, N, Q, D, E, T, A, C, P, Y, M, V, W, I, L, and F. In some embodiments, each N₆₉ is, independently, an amino acid selected from the group consisting of K, R, H, and S. In some embodiments, each N₇₀ is, independently, an amino acid selected from the group consisting of D, E, Q, N, S, H, T, R, K, G, A, C, Y, P, M, V, W, I, F, and L. In some embodiments, each N₇₀ is, independently, an amino acid selected from the group consisting of D, E, Q, and N. In some embodiments, each N₇₁ is, independently, an amino acid selected from the group consisting of A, T, C, G, S, Q, N, P, R, Y, E, K, D, H, M, V, W, I, L, and F. In some embodiments, each N₇₁ is, independently, an amino acid selected from the group consisting of A, T, C, and G.

In some embodiments, the pro-protein signal peptide comprises an amino acid sequence of SEQ ID NO. 75.

In some embodiments, a synthetic pre-protein signal peptide is provided. In some embodiments, the synthetic pre-protein signal peptide comprises an amino acid sequence selected from the group consisting of Formula I, Formula II, Formula III, Formula IV, Formula V, Formula IX, and Formula XIII. In some embodiments, the synthetic pre-protein signal peptide comprises an amino acid sequence of Formula I. In some embodiments, the synthetic pre-protein signal peptide comprises an amino acid sequence of Formula II. In some embodiments, the synthetic pre-protein signal peptide comprises an amino acid sequence of Formula III. In some embodiments, the synthetic pre-protein signal peptide comprises an amino acid sequence of Formula IV. In some embodiments, the synthetic pre-protein signal peptide comprises an amino acid sequence of Formula V. In some embodiments, the synthetic pre-protein signal peptide comprises an amino acid sequence of Formula IX. In some embodiments, the synthetic pre-protein signal peptide comprises an amino acid sequence of Formula XIII.

In some embodiments, the synthetic pre-protein signal peptide comprises an amino acid sequence having at least 70% identity to an amino acid sequence selected from the group consisting of SEQ ID NO. 1, 2, 3, 4, 5, 6, 7, 8, 9, 10, 11, 12, 13, 14, 15, 16, 28, 31, 32, 33, 55, 70, 71, 72, or 73. In some embodiments, the synthetic pre-protein signal peptide comprises an amino acid sequence having at least 70%, at least 75%, at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% identity to an amino acid sequence selected from the group consisting of SEQ ID NO. 1, 2, 3, 4, 5, 6, 7, 8, 9, 10, 11, 12, 13, 14, 15, 16, 28, 31, 32, 33, 55, 70, 71, 72, or 73. In some embodiments, the synthetic pre-protein signal peptide comprises an amino acid sequence selected from the group consisting of SEQ ID NO. 1, 2, 3, 4, 5, 6, 7, 8, 9, 10, 11, 12, 13, 14, 15, 16, 28, 31, 32, 33, 55, 70, 71, 72, or 73. In some embodiments, the pre-protein signal peptide further comprises an amino acid sequence of SEQ ID NO. 68, SEQ ID NO. 69, or Formula XII.

In some embodiments, the synthetic pre-protein signal peptide comprises an amino acid sequence of SEQ ID NO: 1. In some embodiments, the synthetic pre-protein signal peptide comprises an amino acid sequence of SEQ ID NO: 2. In some embodiments, the synthetic pre-protein signal peptide comprises an amino acid sequence of SEQ ID NO: 3. In some embodiments, the synthetic pre-protein signal peptide comprises an amino acid sequence of SEQ ID NO: 4. In some embodiments, the synthetic pre-protein signal peptide comprises an amino acid sequence of SEQ ID NO: 5. In some embodiments, the synthetic pre-protein signal peptide comprises an amino acid sequence of SEQ ID NO: 6. In some embodiments, the synthetic pre-protein signal peptide comprises an amino acid sequence of SEQ ID NO: 7. In some embodiments, the synthetic pre-protein signal peptide comprises an amino acid sequence of SEQ ID NO: 8. In some embodiments, the synthetic pre-protein signal peptide comprises an amino acid sequence of SEQ ID NO: 9. In some embodiments, the synthetic pre-protein signal peptide comprises an amino acid sequence of SEQ ID NO: 10. In some embodiments, the synthetic pre-protein signal peptide comprises an amino acid sequence of SEQ ID NO: 11. In some embodiments, the synthetic pre-protein signal peptide comprises an amino acid sequence of SEQ ID NO: 12. In some embodiments, the synthetic pre-protein signal peptide comprises an amino acid sequence of SEQ ID NO: 13. In some embodiments, the synthetic pre-protein signal peptide comprises an amino acid sequence of SEQ ID NO: 14. In some embodiments, the synthetic pre-protein signal peptide comprises an amino acid sequence of SEQ ID NO: 15. In some embodiments, the synthetic pre-protein signal peptide comprises an amino acid sequence of SEQ ID NO: 16. In some embodiments, the synthetic pre-protein signal peptide comprises an amino acid sequence of SEQ ID NO: 28. In some embodiments, the synthetic pre-protein signal peptide comprises an amino acid sequence of SEQ ID NO: 31. In some embodiments, the synthetic pre-protein signal peptide comprises an amino acid sequence of SEQ ID NO: 32. In some embodiments, the synthetic pre-protein signal peptide comprises an amino acid sequence of SEQ ID NO: 33. In some embodiments, the synthetic pre-protein signal peptide comprises an amino acid sequence of SEQ ID NO: 55. In some embodiments, the synthetic pre-protein signal peptide comprises an amino acid sequence of SEQ ID NO: 70. In some embodiments, the synthetic pre-protein signal peptide comprises an amino acid sequence of SEQ ID NO: 71. In some embodiments, the synthetic pre-protein signal peptide comprises an amino acid sequence of SEQ ID NO: 72. In some embodiments, the synthetic pre-protein signal peptide comprises an amino acid sequence of SEQ ID NO: 73

In some embodiments, a synthetic pro-protein signal peptide is provided. In some embodiments, the synthetic pro-protein signal peptide comprises an amino acid sequence selected from the group consisting of Formula VI, Formula VII, Formula VIII, Formula X, Formula XI, Formula XIV, and Formula XV. In some embodiments, the synthetic pro-protein signal peptide comprises an amino acid sequence of Formula VI. In some embodiments, the synthetic pro-protein signal peptide comprises an amino acid sequence of Formula VII. In some embodiments, the synthetic pro-protein signal peptide comprises an amino acid sequence of Formula VIII. In some embodiments, the synthetic pro-protein signal peptide comprises an amino acid sequence of Formula X. In some embodiments, the synthetic pro-protein signal peptide comprises an amino acid sequence of Formula XI. In some embodiments, the synthetic pro-protein signal peptide comprises an amino acid sequence of Formula XIV. In some embodiments, the synthetic pro-protein signal peptide comprises an amino acid sequence of Formula XV.

In some embodiments, the synthetic pro-protein signal peptide comprises an amino acid sequence having at least 70% identity to an amino acid sequence selected from the group consisting of SEQ ID NO. 17, 18, 19, 20, 21, 22, 23, 24, 25, 27, 29, 34, 35, 36, 37, 38, 56, 57, 58, 74, or 75. In some embodiments, the synthetic pro-protein signal peptide comprises an amino acid sequence having least 70%, at least 75%, at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% identity to an amino acid sequence selected from the group consisting of SEQ ID NO. 17, 18, 19, 20, 21, 22, 23, 24, 25, 27, 29, 34, 35, 36, 37, 38, 56, 57, 58, 74, or 75. In some embodiments, the synthetic pro-protein signal peptide comprises an amino acid sequence selected from the group consisting of SEQ ID NO. 17, 18, 19, 20, 21, 22, 23, 24, 25, 34, 35, 36, 37, 38, 56, 57, 58, 74, and 75. In some embodiments, the pro-protein signal peptide further comprises an amino acid sequence of SEQ ID NO. 68, SEQ ID NO. 69, or Formula XII.

In some embodiments, the synthetic pro-protein signal peptide comprises an amino acid sequence of SEQ ID NO: 17. In some embodiments, the synthetic pro-protein signal peptide comprises an amino acid sequence of SEQ ID NO: 18. In some embodiments, the synthetic pro-protein signal peptide comprises an amino acid sequence of SEQ ID NO: 19. In some embodiments, the synthetic pro-protein signal peptide comprises an amino acid sequence of SEQ ID NO: 20. In some embodiments, the synthetic pro-protein signal peptide comprises an amino acid sequence of SEQ ID NO: 21. In some embodiments, the synthetic pro-protein signal peptide comprises an amino acid sequence of SEQ ID NO: 22. In some embodiments, the synthetic pro-protein signal peptide comprises an amino acid sequence of SEQ ID NO: 23. In some embodiments, the synthetic pro-protein signal peptide comprises an amino acid sequence of SEQ ID NO: 24. In some embodiments, the synthetic pro-protein signal peptide comprises an amino acid sequence of SEQ ID NO: 25. In some embodiments, the synthetic pro-protein signal peptide comprises an amino acid sequence of SEQ ID NO: 27. In some embodiments, the synthetic pro-protein signal peptide comprises an amino acid sequence of SEQ ID NO: 29. In some embodiments, the synthetic pro-protein signal peptide comprises an amino acid sequence of SEQ ID NO: 34. In some embodiments, the synthetic pro-protein signal peptide comprises an amino acid sequence of SEQ ID NO: 35. In some embodiments, the synthetic pro-protein signal peptide comprises an amino acid sequence of SEQ ID NO: 36. In some embodiments, the synthetic pro-protein signal peptide comprises an amino acid sequence of SEQ ID NO: 37. In some embodiments, the synthetic pro-protein signal peptide comprises an amino acid sequence of SEQ ID NO: 38. In some embodiments, the synthetic pro-protein signal peptide comprises an amino acid sequence of SEQ ID NO: 56. In some embodiments, the synthetic pro-protein signal peptide comprises an amino acid sequence of SEQ ID NO: 57. In some embodiments, the synthetic pro-protein signal peptide comprises an amino acid sequence of SEQ ID NO: 58. In some embodiments, the synthetic pro-protein signal peptide comprises an amino acid sequence of SEQ ID NO: 74. In some embodiments, the synthetic pro-protein signal peptide comprises an amino acid sequence of SEQ ID NO: 75.

In some embodiments, a pre-protein plus a pro-protein signal peptide is provided. In some embodiments, the pre-protein plus a pro-protein signal peptide comprises an amino acid sequence having at least 90%, 91%, 92%, 93%, 94%, 95%, 96%, 97%, 98%, 99% or 100% identity to an amino acid sequence of SEQ ID NO: 30.

In some embodiments, a recombinant polypeptide is provided, the recombinant polypeptide comprising a formula of (X₁)ₙ-(Y₁)ₘ-Z₁, wherein X₁ is a synthetic pre-protein signal peptide, Y₁ is a synthetic pro-protein signal peptide, and Z₁ is a payload protein, wherein n is 0 or 1, and m is 0 or 1, wherein n and m cannot concurrently be 0. In some embodiments, n is 0, m is 1, and the recombinant polypeptide comprises a formula of (Y₁)-Z₁. In some embodiments, n is 1, m is 0, and the recombinant polypeptide comprises a formula of (X₁)-Z₁. In some embodiments, n is 1, m is 1, and the recombinant polypeptide comprises a formula of (X₁)-(Y₁)-Z₁.

In some embodiments, the recombinant polypeptide further comprises an amino acid sequence of SEQ ID NO. 68, SEQ ID NO. 69, or Formula XII at the N-terminus of the payload protein Z₁. In some embodiments, the formula of (X₁)ₙ-(Y₁)ₘ-Z₁ could further be written of (X₁)ₙ-(Y₁)ₘ-(K₁)ₚ-Z₁, wherein X₁ is a synthetic pre-protein signal peptide, Y₁ is a synthetic pro-protein signal peptide, K₁ is the a sequence selected from the group consisting of SEQ ID NO. 68, SEQ ID NO. 69, and Formula XII, and Z₁ is a payload protein, wherein n is 0 or 1, m is 0 or 1, and p is 0 or 1, and wherein n and m cannot concurrently be 0. In some embodiments, n is 0, m is 1, p is 0 and the recombinant polypeptide comprises a formula of (Y₁)-Z₁. In some embodiments, n is 0, m is 1, p is 1 and the recombinant polypeptide comprises a formula of (Y₁)-(K₁)-Z₁. In some embodiments, n is 1, m is 0, p is 0 and the recombinant polypeptide comprises a formula of (X₁)-Z₁. In some embodiments, n is 1, m is 0, p is 1 and the recombinant polypeptide comprises a formula of (X₁)-(K₁)-Z₁. In some embodiments, n is 1, m is 1, p is 0 and the recombinant polypeptide comprises a formula of (X₁)-(Y₁)-Z₁. In some embodiments, n is 1, m is 1, p is 1 and the recombinant polypeptide comprises a formula of (X₁)-(Y₁)-(K₁)-Z₁.

In some embodiments, n is 1 and X₁ comprises an amino acid sequence selected from the group consisting of Formula I, Formula II, Formula III, Formula IV, Formula V, Formula IX, and Formula XIII. In some embodiments, X₁ comprises an amino acid sequence of Formula I. In some embodiments, X₁ comprises an amino acid sequence of Formula II. In some embodiments, X₁ comprises an amino acid sequence of Formula III. In some embodiments, X₁ comprises an amino acid sequence of Formula IV. In some embodiments, X₁ comprises an amino acid sequence of Formula V. In some embodiments, X₁ comprises an amino acid sequence of Formula IX. In some embodiments, X₁ comprises an amino acid sequence of Formula XIII. In some embodiments, X₁ comprises an amino acid sequence having at least 70% identity to an amino acid sequence selected from the group consisting of SEQ ID NO. 1, 2, 3, 4, 5, 6, 7, 8, 9, 10, 11, 12, 13, 14, 15, 16, 28, 31, 32, 33, 55, 70, 71, 72, or 73. In some embodiments, X₁ comprises an amino acid sequence having at least 70%, at least 75%, at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% identity to an amino acid sequence selected from the group consisting of SEQ ID NO. 1, 2, 3, 4, 5, 6, 7, 8, 9, 10, 11, 12, 13, 14, 15, 16, 28, 31, 32, 33, 55, 70, 71, 72, or 73. In some embodiments, X₁ comprises an amino acid sequence selected from the group consisting of SEQ ID NO. 1, 2, 3, 4, 5, 6, 7, 8, 9, 10, 11, 12, 13, 14, 15, 16, 28, 31, 32, 33, 55, 70, 71, 72, or 73.

In some embodiments, m is 1 and Y₁ comprises an amino acid sequence selected from the group consisting of Formula VI, Formula VII, Formula VIII, Formula X, Formula XI, Formula XIV, and Formula XV. In some embodiments, Y₁ comprises an amino acid sequence of Formula VI. In some embodiments, Y₁ comprises an amino acid sequence of Formula VII. In some embodiments, Y₁ comprises an amino acid sequence of Formula VIII. In some embodiments, Y₁ comprises an amino acid sequence of Formula X. In some embodiments, Y₁ comprises an amino acid sequence of Formula XI. In some embodiments, Y₁ comprises an amino acid sequence of Formula XIV. In some embodiments, Y₁ comprises an amino acid sequence of Formula XV. In some embodiments, Y₁ comprises an amino acid sequence having at least 70% identity to an amino acid sequence selected from the group consisting of SEQ ID NO. 17, 18, 19, 20, 21, 22, 23, 24, 25, 27, 29, 34, 35, 36, 37, 38, 56, 57, 58, 74, or 75. In some embodiments, Y₁ comprises an amino acid sequence having least 70%, at least 75%, at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% identity to an amino acid sequence selected from the group consisting of SEQ ID NO. 17, 18, 19, 20, 21, 22, 23, 24, 25, 27, 29, 34, 35, 36, 37, 38, 56, 57, 58, 74, or 75. In some embodiments, Y₁ comprises an amino acid sequence selected from the group consisting of SEQ ID NO. 17, 18, 19, 20, 21, 22, 23, 24, 25, 27, 29, 34, 35, 36, 37, 38, 56, 57, 58, 74, or 75.

In some embodiments, X₁ and Y₁ are combined and represented by pre-protein plus a pro-protein signal peptide comprises an amino acid sequence having at least 90%, 91%, 92%, 93%, 94%, 95%, 96%, 97%, 98%, 99% or 100% identity to an amino acid sequence of SEQ ID NO: 30.

In some embodiments, the Z₁ is any peptide or protein. In some embodiments, the payload protein is selected from the group comprising an antiviral, insulin, an incretin, an enzyme, an enzyme inhibitor, a hormone, a cytokine, an antibody, an antimicrobial peptide, a mucosal protein, pesticide, bactericide herbicide, fungicide, nematicide, miticide, plant growth regulator, plant growth stimulator, or fertilizer), a vaccine, a diagnostic protein, a feed conversion enzyme, a flavoring, or a nutritional protein.

In some embodiments, Z₁ comprises an amino acid sequence having at least 70% identity to SEQ ID NO. 59:

(SEQ ID NO. 59)
APVNTTTEDETAQIPAEAVIGYSDLEGDEDVAVLPFSNSINNGLLFINTTIASIAAKEEGVSLD
KREEGEPKSMTNETSDRPLVHFTPNKGWMNDPNGLWYDEKDAKWHLYFQYNPNDTVWGTPLEWG
HATSDDLINWEDQPIAIAPKRNDSGAFSGSMVVDYNNTSGFENDTIDPRQRCVAIWTYNTPESE
EQYISYSLDGGYTFTEYQKNPVLAANSTQFRDPKVFWYEPSQKWIMTAAKSQDYKIEIYSSDDL
KSWKLESAFANEGFLGYQYECPGLIEVPTEQDPSKSYWVMFISINPGAPAGGSFNQYFVGSENG
THFEAFDNQSRVVDFGKDYYALQTFFNTDPTYGSALGIAWASNWEYSAFVPTNPWRSSMSLVRK
FSLNTEYQANPETELINLKAEPILNISNAGPWSRFAINTTLTKANSYNVDLSNSIGTLEFELVY
AVNTTQTISKSVFADLSLWEKGLEDPEEYLRMGFEVSASSFFLDRGNSKVKFVKENPYFTNRMS
VNNQPFKSENDLSYYKVYGLLDQNILELYFNDGDVVSTNTYFMTTGNALGSVNMTTGVDNLFYI
DKFQVREVK

or is substantially similar to SEQ ID NO. 59 or is an active fragment of SEQ ID NO. 59. In some embodiments, Z₁ comprises an amino acid sequence having least 70%, at least 75%, at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% identity to SEQ ID NO. 59. In some embodiments, Z₁ comprises an amino acid sequence of SEQ ID NO. 59.

In some embodiments, Z₁ comprises an amino acid sequence having at least 70% identity to SEQ ID NO. 60:

(SEQ ID NO. 60)
SMTNETSDRPLVHFTPNKGWMNDPNGLWYDEKDAKWHLYFQYNPNDTVWGTPLFWGHATSDDLT
NWEDQPIAIAPKRNDSGAFSGSMVVDYNNTSGFENDTIDPRORCVAIWTYNTPESEEQYISYSL
DGGYTFTEYQKNPVLAANSTOFRDPKVFWYEPSQKWIMTAAKSQDYKIEIYSSDDLKSWKLESA
FANEGFLGYQYECPGLIEVPTEQDPSKSYWVMFISINPGAPAGGSFNQYFVGSENGTHFEAFDN
QSRVVDFGKDYYALQTFFNTDPTYGSALGIAWASNWEYSAFVPTNPWRSSMSLVRKESLNTEYQ
ANPETELINLKAEPILNISNAGPWSRFATNTTLTKANSYNVDLSNSTGTLEFELVYAVNTTQTI
SKSVFADLSLWFKGLEDPEEYLRMGFEVSASSFFLDRGNSKVKFVKENPYFTNRMSVNNOPFKS
ENDLSYYKVYGLLDQNILELYFNDGDVVSTNTYFMTTGNALGSVNMTTGVDNLFYIDKFQVREV
K

or is substantially similar to SEQ ID NO. 60 or is an active fragment of SEQ ID NO. 60. In some embodiments, Z₁ comprises an amino acid sequence having least 70%, at least 75%, at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% identity to SEQ ID NO. 60. In some embodiments, Z₁ comprises an amino acid sequence of SEQ ID NO. 60.

In some embodiments, Z₁ comprises an amino acid sequence having at least 70% identity to SEQ ID NO. 61:

(SEQ ID NO. 61)
KVFERCELARTLKRLGMDGYRGISLANWMCLAKWESGYNTRATNYNAGD
RSTDYGIFQINSRYWCNDGKTPGAVNACQLSCSALLQDNIADAVACAKR
VVRDPQGIRAWVAWRNRCONRDVROYVQGCGV

or is substantially similar to SEQ ID NO. 61 or is an active fragment of SEQ ID NO. 61. In some embodiments, Z₁ comprises an amino acid sequence having least 70%, at least 75%, at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% identity to SEQ ID NO. 61. In some embodiments, Z₁ comprises an amino acid sequence of SEQ ID NO. 61.

In some embodiments, Z₁ comprises an amino acid sequence having at least 70% identity to SEQ ID NO. 62:

(SEQ ID NO. 62)
IKHRLNGFTILEHPDPAKRDLLQDIVTWDDKSLFINGERIMLFSGEVHPFRLPVPSLWLDIFHK
IRALGFNCVSFYIDWALLEGKPGDYRAEGIFALEPFFDAAKEAGIYLIARPGSYINAEVSGGGF
PGWLQRVNGILRSSDEPFLKATDNYIANAAAAVAKAQITNGGPVILYQPENEYSGGCCGVKYPD
ADYMQYVMDQARKADIVVPFISNDASPSGHNAPGSGTSAVDIYGHDSYPLGFDCANPSVWPEGK
LPDNFRTLHLEQSPSTPYSLLEFQAGAFDPWGGPGFEKCYALVNHEFSRVFYRNDLSFGVSTEN
LYMTFGGTNWGNLGHPGGYTSYDYGSPITETRNVTREKYSDIKLLANFVKASPSYLTATPRNLT
TGVYTDTSDLAVTPLIGDSPGSFFVVRHTDYSSQESTSYKLKLPTSAGNLTIPQLEGTLSLNGR
DSKIHVVDYNVSGTNIIYSTAEVFTWKKFDGNKVLVLYGGPKEHHELAIASKSNVTIIEGSDSG
IVSTRKGSSVIIGWDVSSTRRIVQVGDLRVFLLDRNSAYNYWVPELPTEGTSPGFSTSKTTASS
IIVKAGYLLRGAHLDGADLHLTADFNATTPIEVIGAPTGAKNLFVNGEKASHTVDKNGIWSSEV
KYAAPEIKLPGLKDLDWKYLDTLPEIKSSYDDSAWVSADLPKTKNTHRPLDTPTSLYSSDYGFH
TGYLIYRGHFVANGKESEFFIRTQGGSAFGSSVWLNETYLGSWTGADYAMDGNSTYKLSQLESG
KNYVITVVIDNLGLDENWTVGEETMKNPRGILSYKLSGQDASAITWKLIGNLGGEDYQDKVRGP
LNEGGLYAERQGFHQPQPPSESWESGSPLEGLSKPGIGFYTAQFDLDLPKGWDVPLYENFGNNT
QAARAQLYVNGYQYGKFTGNVGPQTSFPVPEGILNYRGTNYVALSLWALESDGAKLGSFELSYT
TPVLIGYGNVESPEQPKYEQRKGAY

or is substantially similar to SEQ ID NO. 62 or is an active fragment of SEQ ID NO. 62. In some embodiments, Z₁ comprises an amino acid sequence having least 70%, at least 75%, at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% identity to SEQ ID NO. 62. In some embodiments, Z₁ comprises an amino acid sequence of SEQ ID NO. 62.

In some embodiments, Z₁ comprises an amino acid sequence having at least 70% identity to SEQ ID NO. 63:

(SEQ ID NO. 63)
EVQLVESGGGLVQPGGSLRLSCAASGFTFSDYWMYWVRQAPGKGLEWVS
EININGLITKYPDSVGRFTISRDNAKNTLYLQMNSLRPEDTAVYYCARS
PSGENRGQGTLVTVSS

or is substantially similar to SEQ ID NO. 63 or is an active fragment of SEQ ID NO. 63. In some embodiments, Z₁ comprises an amino acid sequence having least 70%, at least 75%, at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% identity to SEQ ID NO. 63. In some embodiments, Z₁ comprises an amino acid sequence of SEQ ID NO. 63.

In some embodiments, Z₁ comprises an amino acid sequence having at least 70% identity to SEQ ID NO. 64:

(SEQ ID NO. 64)
IEEGKLVIWINGDKGYNGLAEVGKKFEKDTGIKVTVEHPDKLEEKFPQVAATGDGPDIIFWAHD
RFGGYAQSGLLAEITPDKAFQDKLYPFTWDAVRYNGKLIAYPIAVEALSLIYNKDLLPNPPKTW
EEIPALDKELKAKGKSALMFNLQEPYFTWPLIAADGGYAFKYENGKYDIKDVGVDNAGAKAGLT
FLVDLIKNKHMNADTDYSIAEAAFNKGETAMTINGPWAWSNIDTSKVNYGVTVLPTFKGQPSKP
FVGVLSAGINAASPNKELAKEFLENYLLTDEGLEAVNKDKPLGAVALKSYEEELAKDPRIAATM
ENAQKGEIMPNIPQMSAFWYAVRTAVINAASGRQTVDEALKDAQTRITK

or is substantially similar to SEQ ID NO. 64 or is an active fragment of SEQ ID NO. 64. In some embodiments, Z₁ comprises an amino acid sequence having least 70%, at least 75%, at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% identity to SEQ ID NO. 64. In some embodiments, Z₁ comprises an amino acid sequence of SEQ ID NO. 64.

In some embodiments, Z₁ comprises an amino acid sequence having at least 70% identity to SEQ ID NO. 65:

(SEQ ID NO. 65)
AQSEPELKLESVVIVSRHGVRAPTKATQLMQDVTPDAWPTWPVKLGELTPRGGELLAYLGHYWR
QRLVADGLLPKCGCPQSGQVAILADVDERTRKTGEAFAAGLAPDCAITVHTQADTSSPDPLENP
LKTGVCOLDNANVIDAILERAGGSLADFTGHYQTAFRELERVLNFPQSNLCLKREKQDESCSLI
QALPSELKVSADCVSLIGAVSLASMLTEIFLLQQAQGMPEPGWGRITDSHOWNTLLSLHNAQFD
LLQRTPEVARSRATPLLDLIKTALTPHPPQKQAYGVTLPTSVLFLAGHDINLANLGGALELNWT
LPGQPDNTPPGGELVFERWRRLSDNSQWIQVSLVFQTLQQMRDKTPLSLNTPPGEVKLTLAGCE
ERNAQGMCSLAGFTQIVNEARIPACSL

or is substantially similar to SEQ ID NO. 65 or is an active fragment of SEQ ID NO. 65. In some embodiments, Z₁ comprises an amino acid sequence having least 70%, at least 75%, at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% identity to SEQ ID NO. 65. In some embodiments, Z₁ comprises an amino acid sequence of SEQ ID NO. 65.

In some embodiments, Z₁ comprises an amino acid sequence having at least 70% identity to SEQ ID NO. 66:

(SEQ ID NO. 66)
FVNQHLCGSHLVEALYLVCGERGFFYTPKEWKGIVEQCCTSICSLYQLE
NYCN

or is substantially similar to SEQ ID NO. 66 or is an active fragment of SEQ ID NO. 66. In some embodiments, Z₁ comprises an amino acid sequence having least 70%, at least 75%, at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% identity to SEQ ID NO. 66. In some embodiments, Z₁ comprises an amino acid sequence of SEQ ID NO. 66.

In some embodiments, Z₁ comprises an amino acid sequence having at least 70% identity to SEQ ID NO. 67:

(SEQ ID NO. 67)
GPETLCGAELVDALQFVCGPRGFYFNKPTGYGSSIRRAPQTGIVDECCF
RSCDLRRLEMYCAPLKPTKAARSIRAQRHTDMPKTQKEVHLKNTSRGSA
GNKTYRM

or is substantially similar to SEQ ID NO. 67 or is an active fragment of SEQ ID NO. 67. In some embodiments, Z₁ comprises an amino acid sequence having least 70%, at least 75%, at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% identity to SEQ ID NO. 67. In some embodiments, Z₁ comprises an amino acid sequence of SEQ ID NO. 67.

In some embodiments, Z₁ comprises an amino acid sequence having at least 70% identity to SEQ ID NO. 85:

(SEQ ID NO. 85)
KVFERCELARTLKRLGMDGYRGISLANWMCLAKWESGYNTRATNYNAGD
RSTDYGIFQINSRYWCNDGKTPGAVNACQLSCSALLQDNIADAVACAKR
VVRDPQGIRAWVAWRNRCQNRDVRQYVQGCGV

or is substantially similar to SEQ ID NO. 85 or is an active fragment of SEQ ID NO. 85. In some embodiments, Z₁ comprises an amino acid sequence having least 70%, at least 75%, at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% identity to SEQ ID NO. 85. In some embodiments, Z₁ comprises an amino acid sequence of SEQ ID NO. 85.

In any of the embodiments herein, Z₁ may further comprise an affinity tag. The affinity tag may be utilized, for example, for protein purification or detection. The affinity tag may be utilized for any method known in the art for which affinity tags are utilized. Affinity tags are known in the art, and any such affinity tag may be utilized. Non-limiting examples of affinity tags that may be utilized include 6×HIS (SEQ ID NO: 105), FLAG, GST, MBP, a streptavidin peptide, GFP, and the like. In some embodiments, any peptide sequence that can be utilized for purification or detection may be utilized.

In some embodiments, the recombinant polypeptide comprises a formula of (X₁)ₙ-(Y₁)ₘ-Z₁, wherein n is 0 or 1 and m is 0 or 1, wherein n and m cannot concurrently be 0, wherein X₁ comprises an amino acid sequence selected from the group consisting of SEQ ID NO1, 2, 3, 4, 5, 6, 7, 8, 9, 10, 11, 12, 13, 14, 15, 16, 28, 31, 32, 33, 55, 70, 71, 72, or 73, Y₁ comprises an amino acid sequence selected from the group consisting of SEQ ID NO. 17, 18, 19, 20, 21, 22, 23, 24, 25, 27, 29, 34, 35, 36, 37, 38, 56, 57, 58, 74, or 75, and Z₁ comprises an amino acid sequence selected from the group consisting of SEQ ID NO. 59, 60, 61, 62, 63, 64, 65, 66, 67, and 85. In some embodiments, the components X₁, Y₁, and Z₁ are fused directly. In some embodiments, the components X₁, Y₁, and Z₁, are fused indirectly via, for example, a peptide linker as provided for herein.

In some embodiments, the recombinant polypeptide further comprises an amino acid sequence of SEQ ID NO. 68 at the N-terminus of the payload protein Z₁. In some embodiments, the formula of (X₁)ₙ-(Y₁)ₘ-Z₁ could further be written of (X₁)ₙ-(Y₁)ₘ-(K₁)ₚ-Z₁, wherein X₁ is a synthetic pre-protein signal peptide, Y₁ is a synthetic pro-protein signal peptide, K₁ is a sequence selected from the group consisting of SEQ ID NO. 68, SEQ ID NO. 69, and Formula XII, and Z₁ is a payload protein, wherein n is 0 or 1, m is 0 or 1, and p is 0 or 1, and wherein n and m cannot concurrently be 0. In some embodiments, n is 0, m is 1, p is 0 and the recombinant polypeptide comprises a formula of (Y₁)-Z₁. In some embodiments, n is 0, m is 1, p is 1 and the recombinant polypeptide comprises a formula of (Y₁)-(K₁)-Z₁. In some embodiments, n is 1, m is 0, p is 0 and the recombinant polypeptide comprises a formula of (X₁)-Z₁. In some embodiments, n is 1, m is 0, p is 1 and the recombinant polypeptide comprises a formula of (X₁)-(K₁)-Z₁. In some embodiments, n is 1, m is 1, p is 0 and the recombinant polypeptide comprises a formula of (X₁)-(Y₁)-Z₁. In some embodiments, n is 1, m is 1, p is 1 and the recombinant polypeptide comprises a formula of (X₁)-(Y₁)-(K₁)-Z₁.

In some embodiments, a nucleic acid is provided. In some embodiments, the nucleic acid encodes for a recombinant polypeptide as provided for herein. In some embodiments, the recombinant polypeptide comprises a synthetic signal peptide and a payload protein. In some embodiments, the synthetic signal peptide is as provided for herein. In some embodiments, the payload protein is as provided for herein.

In some embodiments, an engineered yeast is provided. In some embodiments, the engineered yeast is genetically modified with a nucleic acid encoding a recombinant polypeptide having a formula of (X₁)ₙ-(Y₁)ₘ-Z₁, wherein X₁ is a synthetic pre-protein signal peptide, Y₁ is a synthetic pro-protein signal peptide, Z₁ is a payload protein, n is 0 or 1, m is 0 or 1, and n and m cannot concurrently be 0.

In some embodiments, the recombinant polypeptide further comprises an amino acid sequence of SEQ ID NO. 68 at the N-terminus of the payload protein Z₁. In some embodiments, the formula of (X₁)ₙ-(Y₁)ₘ-Z₁ could further be written of (X₁)ₙ-(Y₁)ₘ-(K₁)ₚ-Z₁, wherein X₁ is a synthetic pre-protein signal peptide, Y₁ is a synthetic pro-protein signal peptide, K₁ is a sequence selected from the group consisting of SEQ ID NO. 68, SEQ ID NO. 69, and Formula XII, and Z₁ is a payload protein, wherein n is 0 or 1, m is 0 or 1, and p is 0 or 1, and wherein n and m cannot concurrently be 0. In some embodiments, n is 0, m is 1, p is 0 and the recombinant polypeptide comprises a formula of (Y₁)-Z₁. In some embodiments, n is 0, m is 1, p is 1 and the recombinant polypeptide comprises a formula of (Y₁)-(K₁)-Z₁. In some embodiments, n is 1, m is 0, p is 0 and the recombinant polypeptide comprises a formula of (X₁)-Z₁. In some embodiments, n is 1, m is 0, p is 1 and the recombinant polypeptide comprises a formula of (X₁)-(K₁)-Z₁. In some embodiments, n is 1, m is 1, p is 0 and the recombinant polypeptide comprises a formula of (X₁)-(Y₁)-Z₁. In some embodiments, n is 1, m is 1, p is 1 and the recombinant polypeptide comprises a formula of (X₁)-(Y₁)-(K₁)-Z₁

In some embodiments, n is 1 and X₁ comprises an amino acid sequence selected from the group consisting of Formula I, Formula II, Formula III, Formula IV, Formula V, Formula IX, and Formula XIII. In some embodiments, X₁ comprises an amino acid sequence of Formula I. In some embodiments, X₁ comprises an amino acid sequence of Formula II. In some embodiments, X₁ comprises an amino acid sequence of Formula III. In some embodiments, X₁ comprises an amino acid sequence of Formula IV. In some embodiments, X₁ comprises an amino acid sequence of Formula V. In some embodiments, X₁ comprises an amino acid sequence of Formula IX. In some embodiments, X₁ comprises an amino acid sequence of Formula XIII. In some embodiments, X₁ comprises an amino acid sequence having at least 70% identity to an amino acid sequence selected from the group consisting of SEQ ID NO. 1, 2, 3, 4, 5, 6, 7, 8, 9, 10, 11, 12, 13, 14, 15, 16, 28, 31, 32, 33, 55, 70, 71, 72, or 73. In some embodiments, X₁ comprises an amino acid sequence having at least 70%, at least 75%, at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% identity to an amino acid sequence selected from the group consisting of SEQ ID NO. 1, 2, 3, 4, 5, 6, 7, 8, 9, 10, 11, 12, 13, 14, 15, 16, 28, 31, 32, 33, 55, 70, 71, 72, or 73. In some embodiments, X₁ comprises an amino acid sequence selected from the group consisting of SEQ ID NO. 1, 2, 3, 4, 5, 6, 7, 8, 9, 10, 11, 12, 13, 14, 15, 16, 28, 31, 32, 33, 55, 70, 71, 72, or 73.

In some embodiments, m is 1 and Y₁ comprises an amino acid sequence selected from the group consisting of Formula VI, Formula VII, Formula VIII, Formula X, Formula XI, Formula XIV, and Formula XV. In some embodiments, Y₁ comprises an amino acid sequence of Formula VI. In some embodiments, Y₁ comprises an amino acid sequence of Formula VII. In some embodiments, Y₁ comprises an amino acid sequence of Formula VIII. In some embodiments, Y₁ comprises an amino acid sequence of Formula X. In some embodiments, Y₁ comprises an amino acid sequence of Formula XI. In some embodiments, Y₁ comprises an amino acid sequence of Formula XIV. In some embodiments, Y₁ comprises an amino acid sequence of Formula XV. In some embodiments, Y₁ comprises an amino acid sequence having at least 70% identity to an amino acid sequence selected from the group consisting of SEQ ID NO. 17, 18, 19, 20, 21, 22, 23, 24, 25, 27, 29, 34, 35, 36, 37, 38, 56, 57, 58, 74, or 75. In some embodiments, Y₁ comprises an amino acid sequence having least 70%, at least 75%, at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% identity to an amino acid sequence selected from the group consisting of SEQ ID NO. 17, 18, 19, 20, 21, 22, 23, 24, 25, 27, 29, 34, 35, 36, 37, 38, 56, 57, 58, 74, or 75. In some embodiments, Y₁ comprises an amino acid sequence selected from the group consisting of SEQ ID NO. 17, 18, 19, 20, 21, 22, 23, 24, 25, 27, 29, 34, 35, 36, 37, 38, 56, 57, 58, 74, or 75.

In some embodiments, the Z₁ is any peptide or protein. In some embodiments, the payload protein is selected from the group comprising an antiviral, insulin, an incretin, an enzyme, an enzyme inhibitor, a hormone, a cytokine, an antibody, an antimicrobial peptide, a mucosal protein, pesticide, bactericide herbicide, fungicide, nematicide, miticide, plant growth regulator, plant growth stimulator, or fertilizer), a vaccine, a diagnostic protein, a feed conversion enzyme, a flavoring, or a nutritional protein.

In some embodiments, Z₁ comprises an amino acid sequence having at least 70% identity to an amino acid sequence selected from the group consisting of SEQ ID NO. 59, 60, 61, 62, 63, 64, 65, 66, and 67. In some embodiments, Z₁ comprises an amino acid sequence having least 70%, at least 75%, at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% identity to an amino acid sequence selected from the group consisting of SEQ ID NO. 59, 60, 61, 62, 63, 64, 65, 66, 67, and 85. In some embodiments, Z₁ comprises an amino acid sequence selected from the group consisting of SEQ ID NO. 59, 60, 61, 62, 63, 64, 65, 66, 67, and 85.

In some embodiments, the components X₁, Y₁, and Z₁ are fused directly. In some embodiments, the components X₁, Y₁, and Z₁, are fused indirectly via, for example, a peptide linker as provided for herein.

In some embodiments, the identity of X₁, Y₁, and Z₁ are influenced by the strain of yeast utilized. In some embodiments, the strain of yeast is any yeast as provided for herein. In some embodiments, the yeast is selected from the group consisting of Kluyveromyces, Pichia, Saccharomyces, Trichoderma, and Aspergillus. Specific yeast, X₁, Y₁, and Z₁ combinations are described and provided for below. It is to be understood that the embodiments provided below are merely exemplary and are not meant to limit the scope of the invention in any way. Thus, although a particular embodiment may be silent on the use of a particular pre or pro protein SEQ ID NO, this is not to be construed as the particular SEQ ID NO. being excluded from use in the particular yeast. Further, although a particular embodiment may be silent on the inclusion of any synthetic pre or pro protein signal peptides, this is not to be construed as the pre or pro protein signal peptides are excluded from use in the particular yeast. For example, if a recombinant polypeptide is described for use in a particular yeast and the recombinant polypeptide is said to comprise a synthetic pre-protein signal peptide domain and a payload protein domain, this is not to be construed as a synthetic pro-protein signal domain cannot be included for the particular yeast. Likewise, if a recombinant polypeptide is described for use is a particular yeast and the recombinant polypeptide is said to comprise a synthetic pro-protein signal peptide domain and a payload protein domain, this is not to be construed as a synthetic pre-protein signal domain cannot be included for the particular yeast.

Synthetic Pre-Protein Signal Peptides and their Use in Kluyveromyces Yeast

In some embodiments, a synthetic pre-protein signal peptide that may be fused to a payload protein to facilitate secretion of the payload protein from Kluyveromyces yeast (e.g., K. lactis) is provided. In some embodiments, Kluyveromyces yeast (e.g., K. lactis) may be genetically modified with a nucleic acid molecule encoding for expression of a recombinant polypeptide comprising a synthetic pre-protein signal peptide fused either directly or indirectly to a payload protein. In some embodiments, the synthetic pre-protein signal peptide comprises an amino acid sequence of Formula I or SEQ ID NO. 1. In some embodiments, the nucleic acid molecule is any nucleic acid molecule encoding for a peptide comprising an amino acid sequence of Formula I or SEQ ID NO. 1. For example, SEQ ID NO. 39 may be used to encode for the synthetic pre-protein signal peptide comprising an amino acid sequence of SEQ ID NO. 1. It is to be understood that the previous example is not meant to be limiting in any way. One who is skilled in the art will understand how to develop a suitable nucleotide sequence that will induce expression of a synthetic signal peptide comprising an amino acid sequence of Formula I or SEQ ID NO. 1. In some embodiments, a signal peptide comprising an amino acid sequence of Formula I or SEQ ID NO. 1 may be fused directly or indirectly to a native constitutive pro-protein signal peptide or a synthetic signal peptide as disclosed herein.

In some embodiments, a recombinant polypeptide comprising a synthetic pre-protein signal peptide comprising an amino acid sequence of Formula I or SEQ ID NO. 1 and a payload protein is provided. In some embodiments, inclusion of the pre-protein signal peptide comprising an amino acid sequence of Formula I or SEQ ID NO. 1 will result in the payload protein being more readily secreted by the yeast in which it is produced. Accordingly, in another embodiment, a method of producing a payload protein with Kluyveromyces yeast (e.g., K. lactis) is provided, the method comprising providing a nucleic acid molecule encoding a recombinant polypeptide comprising a payload protein and a synthetic signal peptide comprising an amino acid sequence of Formula I SEQ ID NO. 1; genetically modifying the Kluyveromyces yeast (e.g., K. lactis) with the nucleic acid molecule, thereby generating engineered yeast; and culturing the engineered yeast under effective conditions to express the recombinant polypeptide. In some embodiments, the nucleic acid molecule encoding the synthetic signal peptide of SEQ ID NO. 1 is SEQ ID NO. 39. In some embodiments, the nucleic acid molecule encoding the synthetic signal peptide amino acid of Formula I or SEQ ID NO. 1 is any nucleic acid molecule encoding for said amino acid sequences.

In some embodiments, a method of increasing extracellular secretion of a payload protein from Kluyveromyces yeast (e.g., K. lactis) is provided, the method comprising providing a nucleic acid encoding a recombinant polypeptide comprising a payload protein and a synthetic pre-protein signal peptide, genetically modifying the Kluyveromyces yeast (e.g., K. lactis) with the nucleic acid, thereby generating an engineered yeast, and culturing the engineered yeast under effective conditions to produce and secrete an increased amount of payload protein when compared to the amount of payload protein secreted by Kluyveromyces yeast (e.g., K. lactis) using a recombinant polypeptide comprising the payload protein and signal peptide α-MF or any other commonly utilized signal peptide such as SUC2, PHO5, or HSA. In some embodiments, the synthetic pre-protein signal peptide comprises an amino acid sequence of Formula I or SEQ ID NO. 1. In some embodiments, the synthetic pre-protein signal peptide further comprises a native pro-protein signal peptide. In some embodiments, the synthetic pre-protein signal peptide further comprises a synthetic pro-protein signal peptide. In some embodiments, the synthetic pre-protein signal peptide is fused directly to the payload protein. In some embodiments, the synthetic pre-protein signal peptide is connected to the payload protein via a peptide linker as provided for herein.

In some embodiments, an engineered Kluyveromyces yeast (e.g., K. lactis) is provided, wherein the yeast is genetically modified with a nucleic acid molecule encoding the expression of a recombinant polypeptide comprising a synthetic pre-protein signal peptide fused directly or indirectly to a payload protein. In some embodiments, the synthetic pre-protein signal peptide comprises an amino acid sequence of Formula I or SEQ ID NO. 1. In some embodiments, the synthetic pre-protein signal peptide further comprises a native pro-protein signal peptide. In some embodiments, the synthetic pre-protein signal peptide further comprises a synthetic pro-protein signal peptide as provided for herein. In some embodiments, the synthetic pre-protein signal peptide is fused directly to the payload protein. In some embodiments, the synthetic pre-protein signal peptide is indirectly fused to the payload protein via a connecting linker peptide sequence as provided for herein. In some embodiments, the nucleic acid molecule used to encode the synthetic pre-protein signal peptide comprising an amino acid sequence of SEQ ID NO. 1 is given by SEQ ID NO. 39. In some embodiments, the nucleic acid molecule used to encode the synthetic pre-protein signal peptide comprising an amino acid sequence of Formula I or SEQ ID NO. 1 is any nucleic acid molecule encoding for said amino acid sequence.

In some embodiments, the payload protein may be any peptide or protein. In some embodiments, the payload protein is selected from the group comprising an enzyme (e.g., invertase, isomaltase, lactase, lysozyme, An-PEP), a growth factor (e.g., IGF-1), insulin, an incretin (e.g., GLP-1, GLP-2, leptin, apelin, ghrelin, PYY, nesfatin), a cytokine, an antibody, an antimicrobial peptide), a mucosal protein (e.g., trefoil factor, Reg3 protein, superoxide dismutase), an agricultural product (e.g., pesticide, bactericide herbicide, fungicide, nematicide, miticide, plant growth regulator, plant growth stimulator, or fertilizer), a vaccine, a diagnostic protein, a feed conversion enzyme, a flavoring, or a nutritional protein. The examples listed are provided for clarity only and are not meant to be limiting in any way. Thus, for example, the current disclosure is not limited to IGF-1 for “growth factor”, but rather encompasses and includes all growth factors known in the art.

Synthetic Pre-Protein Signal Peptides and their Use in a Pichia Yeast

In some embodiments, a synthetic pre-protein signal peptide for use in the yeast species Pichia (e.g., P. pastoris) is provided. In some embodiments, the Pichia yeast may be genetically modified with a nucleic acid molecule encoding the expression of a recombinant polypeptide comprising a synthetic pre-protein signal peptide fused directly or indirectly to a payload protein. In some embodiments, the synthetic pre-protein signal comprises an amino acid sequence represented by Formula II or SEQ ID NOs. 2, 3, 4, 5, 6, or 7. In some embodiments, the synthetic pre-protein signal peptide is fused directly to the payload protein. In some embodiments, the synthetic pre-protein signal peptide is fused indirectly to the payload protein, connecting via a peptide linker as provided for herein. In some embodiments, any nucleic acid encoding for Formula II or SEQ ID NO. 2, 3, 4, 5, 6 or 7 may be utilized to induce expression of the synthetic signal peptide. One of skill in the art will understand how to develop a suitable nucleotide sequence that will induce expression of a synthetic pre-protein signal represented by Formula II or SEQ ID NO. 2, 3, 4, 5, 6, or 7. In some embodiments, the synthetic pre-protein signal peptide of Formula II or SEQ ID NO. 2, 3, 4, 5, 6, or 7 may further be fused directly or indirectly to a native constitutive pro-protein signal peptide or a synthetic signal peptide as disclosed herein. In some embodiments, the synthetic pre-protein signal peptide is further fused to a native constitutive pro-protein signal peptide. In some embodiments, the synthetic pre-protein signal peptide is further fused to a synthetic signal peptide as disclosed herein. In some embodiments, the synthetic pre-protein signal peptide of Formula II or SEQ ID NO. 2, 3, 4, 5, 6, or 7 is further fused to a synthetic pro-protein signal peptide selected from the group consisting of SEQ ID NO. 17, 18, 19, 20, 21, 22, 23, 24, 25, 34, 35, 36, 37, 38, 56, 57, and 58. In some embodiments, the synthetic pre-protein signal peptide of Formula II or SEQ ID NO. 2, 3, 4, 5, 6, or 7 is further fused to a synthetic pro-protein signal peptide as represented by SEQ. ID NO. 17.

In some embodiments, a recombinant polypeptide comprising a synthetic pre-protein signal peptide comprising an amino acid sequence of Formula II or SEQ ID NO. 2, 3, 4, 5, 6, or 7 and a payload protein is provided. In some embodiments, inclusion of the pre-protein signal peptide comprising an amino acid sequence of Formula II or SEQ ID NO. 2, 3, 4, 5, 6, or 7 will result in the payload protein being more readily secreted by the yeast in which it is produced. Accordingly, in some embodiments, a method of producing a payload protein with Pichia yeast (e.g., P. pastoris) is provided, the method comprising providing a nucleic acid molecule encoding a recombinant polypeptide comprising a payload protein and a synthetic pre-protein signal peptide comprising an amino acid sequence of Formula II or SEQ ID NO. 2, 3, 4, 5, 6, or 7; genetically modifying the a Pichia yeast (e.g., P. pastoris) with the nucleic acid molecule, thereby generating engineered yeast; and culturing the engineered yeast under effective conditions to express the recombinant polypeptide. In some embodiments, the nucleic acid molecule encoding for the amino acid sequence of Formula II or SEQ ID NO. 2, 3, 4, 5, 6, or 7 is any nucleic acid molecule encoding for said amino acid sequence.

In some embodiments, a method of increasing extracellular secretion of a payload protein from a Pichia yeast is provided, the method comprising providing a nucleic acid molecule encoding a recombinant polypeptide comprising a payload protein and a synthetic pre-protein signal peptide; genetically modifying the a Pichia yeast (e.g., P. pastoris) with the nucleic acid, thereby generating an engineered yeast, and culturing the engineered yeast under effective conditions to produce and secrete an increased amount of payload protein when compared to the amount of payload protein secreted by a Pichia yeast genetically modified to express a recombinant polypeptide comprising the payload protein and pre-protein signal peptide α-MF (α-MF comprising an amino acid sequence represented by SEQ ID NO. 27). In some embodiments, the synthetic pre-protein signal peptide comprises an amino acid sequence of Formula II or SEQ ID NO. 2, 3, 4, 5, 6, or 7. In some embodiments, the synthetic pre-protein signal peptide further comprises a native pro-protein signal peptide. In some embodiments, the synthetic pre-protein signal peptide further comprises a synthetic pro-protein signal peptide as provided for herein. In some embodiments, the synthetic pre-protein signal peptide is fused directly to the payload protein. In some embodiments, the synthetic pre-protein signal peptide is fused indirectly to the payload protein via, for example, a peptide linker as provided for herein.

In some embodiments, an engineered Pichia yeast (e.g., P. pastoris) is provided, wherein the yeast is genetically modified with a nucleic acid encoding the expression of a recombinant polypeptide comprising a synthetic pre-protein signal peptide fused directly or indirectly to a payload protein. In some embodiments, the synthetic pre-protein signal peptide comprises an amino acid sequence of Formula II or SEQ ID NO. 2, 3, 4, 5, 6, or 7. In some embodiments, the synthetic pre-protein signal peptide further comprises a native pro-protein signal peptide. In some embodiments, the synthetic pre-protein signal peptide further comprises a synthetic pro-protein signal peptide as provided for herein. In some embodiments, the synthetic pre-protein signal peptide is fused directly to the payload protein. In some embodiments, the synthetic pre-protein signal peptide is fused indirectly to the payload protein via, for example, a peptide linker as provided for herein. In some embodiments, the payload protein may be any peptide or protein. In some embodiments, the payload protein is selected from the group comprising an enzyme (e.g., invertase, isomaltase, lactase, lysozyme, An-PEP), a growth factor (e.g., IGF-1), insulin, an incretin (e.g., GLP-1, GLP-2, leptin, apelin, ghrelin, PYY, nesfatin), a cytokine, an antibody, an antimicrobial peptide), a mucosal protein (e.g., trefoil factor, Reg3 protein, superoxide dismutase), an agricultural product (e.g., pesticide, bactericide herbicide, fungicide, nematicide, miticide, plant growth regulator, plant growth stimulator, or fertilizer), a vaccine, a diagnostic protein, a feed conversion enzyme, a flavoring, or a nutritional protein. The examples listed are provided for clarity only and are not meant to be limiting in any way. Thus, for example, the current disclosure is not limited to IGF-1 for “growth factor”, but rather encompasses and includes all growth factors known in the art.

Synthetic Pre-Protein Signal Peptides and their Use in Saccharomyces Yeast

In another embodiment, a synthetic pre-protein signal peptide for use in the yeast species Saccharomyces (e.g., S. boulardii or S. cerevisiae) is provided. In some embodiments, S. cerevisiae yeast may be genetically modified with a nucleic acid molecule encoding the expression of a recombinant polypeptide comprising a synthetic pre-protein signal peptide fused directly or indirectly to a payload protein. In some embodiments, the synthetic pre-protein signal peptide comprises an amino acid sequence of Formula III, Formula IV, Formula V, or SEQ ID NO. 8, 9, 10, 11, 12, 13, 14, 15, or 16. In some embodiments, the synthetic pre-protein signal peptide is fused directly to the payload protein. In some embodiments, the synthetic pre-protein signal peptide is fused indirectly to the payload protein via, for example, a peptide linker as provided for herein. In some embodiments, any nucleic acid encoding for Formula III, Formula IV, Formula V, or SEQ ID NO. 8, 9, 10, 11, 12, 13, 14, 15, or 16 may be utilized to induce expression of the synthetic pre-protein signal peptide. One of skill in the art will understand how to develop a suitable nucleic acid that will induce expression of a synthetic signal peptide comprising an amino acid sequence of Formula III, Formula IV, Formula V, or SEQ ID NO. 8, 9, 10, 11, 12, 13, 14, 15, or 16. In some embodiments, a pre-protein signal peptide comprising an amino acid sequence of Formula III, Formula IV, Formula V, or SEQ ID NO. 8, 9, 10, 11, 12, 13, 14, 15, or 16 may be fused directly or indirectly to a native constitutive pro-protein signal peptide. In some embodiments, a pre-protein signal peptide comprising an amino acid sequence of Formula III, Formula IV, Formula V, or SEQ ID NO. 8, 9, 10, 11, 12, 13, 14, 15, or 16 may be fused directly or indirectly to a synthetic signal peptide as disclosed herein, such as Formula VI, Formula VII, Formula VIII or SEQ ID NO. 17, 18, 19, 20, 21, 22, 23, or 24. In some embodiments, the synthetic pre-protein signal peptide is fused directly to the native or synthetic pro-protein signal peptide. In some embodiments, the synthetic pre-protein signal peptide is fused indirectly to the native or synthetic pro-protein signal peptide via, for example, a peptide linker as provided for herein.

In some embodiments, a recombinant polypeptide comprising a synthetic pre-protein signal peptide comprising an amino acid sequence of Formula III, Formula IV, Formula V or SEQ ID NO. 8, 9, 10, 11, 12, 13, 14, 15, or 16 and a payload protein is provided. In some embodiments, inclusion of the synthetic pre-protein signal peptide comprising an amino acid sequence of Formula III, Formula IV, Formula V, or SEQ ID NO. 8, 9, 10, 11, 12, 13, 14, 15, or 16 will result in the payload protein being more readily secreted by the yeast in which it is produced. Accordingly, in another embodiment, a method of producing a payload protein with Saccharomyces yeast is provided, the method comprising providing a nucleic acid molecule encoding a recombinant polypeptide comprising a payload protein and a synthetic pre-protein signal peptide comprising an amino acid sequence of Formula III, Formula IV, Formula V, or SEQ ID NO. 8, 9, 10, 11, 12, 13, 14, 15, or 16; genetically modifying the Saccharomyces yeast with the nucleic acid, thereby generating engineered yeast; and culturing the engineered yeast under effective conditions to express the recombinant polypeptide. In some embodiments, the nucleic acid molecule encoding for the amino acid sequence of Formula III, Formula IV, Formula V, or SEQ ID NO. 8, 9, 10, 11, 12, 13, 14, 15, or 16 is any nucleic acid molecule encoding for said amino acid sequence.

In some embodiments, a method of increasing extracellular secretion of a payload protein from Saccharomyces yeast is provided, the method comprising providing a nucleic acid encoding a recombinant polypeptide comprising a payload protein and a synthetic pre-protein signal peptide; genetically modifying the Saccharomyces yeast with the nucleic acid, thereby generating an engineered yeast, and culturing the engineered yeast under effective conditions to produce and secrete an increased amount of payload protein when compared to the amount of payload protein secreted by Saccharomyces yeast genetically modified to express a recombinant polypeptide comprising the payload protein and pre-protein signal peptide α-MF or Yeast Aspartic Protease 3 (YAP). In some embodiments, the synthetic pre-protein signal peptide comprises an amino acid sequence of Formula III, Formula IV, Formula V, or SEQ ID NO. 8, 9, 10, 11, 12, 13, 14, 15, or 16. In some embodiments, the synthetic pre-protein signal peptide further comprises a native pro-protein signal peptide. In some embodiments, the synthetic pre-protein signal peptide further comprises a synthetic pro-protein signal peptide as provided for herein. In some embodiments, the synthetic pre-protein signal peptide is fused directly to the payload protein. In some embodiments, the synthetic pre-protein signal peptide is fused indirectly to the payload protein via, for example, a peptide linker as provided for herein.

In some embodiments, an engineered Saccharomyces yeast (e.g., S. boulardii or S. cerevisiae) is provided, wherein the yeast is genetically modified with a nucleic acid molecule encoding the expression of a recombinant polypeptide comprising a synthetic pre-protein signal peptide fused directly or indirectly to a payload protein. In some embodiments, the synthetic pre-protein signal peptide comprises an amino acid sequence of Formula III, Formula IV, Formula V, or SEQ ID NO. 8, 9, 10, 11, 12, 13, 14, 15, or 16. In some embodiments, the synthetic pre-protein signal peptide further comprises a native pro-protein signal peptide. In some embodiments, the synthetic pre-protein signal peptide further comprises a synthetic pro-protein signal peptide as provided for herein. In some embodiments, the synthetic pre-protein signal peptide is fused directly to the payload protein. In some embodiments, the synthetic pre-protein signal peptide is fused indirectly to the payload protein via, for example, a peptide linker as provided for herein. In some embodiments, the payload protein may be any peptide or protein. In some embodiments, the payload protein is selected from the group comprising an enzyme (e.g., invertase, isomaltase, lactase, lysozyme, An-PEP), a growth factor (e.g., IGF-1), insulin, an incretin (e.g., GLP-1, GLP-2, leptin, apelin, ghrelin, PYY, nesfatin), a cytokine, an antibody, an antimicrobial peptide), a mucosal protein (e.g., trefoil factor, Reg3 protein, superoxide dismutase), an agricultural product (e.g., pesticide, bactericide herbicide, fungicide, nematicide, miticide, plant growth regulator, plant growth stimulator, or fertilizer), a vaccine, a diagnostic protein, a feed conversion enzyme, a flavoring, or a nutritional protein. The examples listed are provided for clarity only and are not meant to be limiting in any way. Thus, for example, the current disclosure is not limited to IGF-1 for “growth factor”, but rather encompasses and includes all growth factors known in the art.

Synthetic Pre-Protein Signal Peptides and their Use in Trichoderma Yeast

In some embodiments, a synthetic pre-protein signal peptide for use in the yeast species Trichoderma (e.g., T. reesei or T. viride) is provided. In some embodiments, Trichoderma yeast may be genetically modified with a nucleic acid molecule encoding the expression of a recombinant polypeptide comprising a synthetic pre-protein signal peptide fused directly or indirectly to a payload protein. In some embodiments, the synthetic pre-protein signal peptide comprises an amino acid sequence of Formula IX or SEQ ID NO. 31, 32, or 33. In some embodiments, the synthetic pre-protein signal peptide is fused directly to the payload protein. In some embodiments, the synthetic pre-protein signal peptide is fused indirectly to the payload protein via, for example, a peptide linker as provided for herein. In some embodiments, any nucleic acid molecule encoding for Formula IX or SEQ ID NO. 31, 32, or 33 may be utilized to induce expression of the synthetic signal peptide. One of skill in the art will understand how to develop a suitable nucleotide sequence that will induce expression of a synthetic pre-protein signal peptide comprising an amino acid sequence of Formula IX or SEQ ID NO. 31, 32, or 33. In some embodiments, a synthetic pre-protein signal peptide comprising an amino acid sequence of Formula IX or SEQ ID NO. 31, 32, or 33 may further be fused directly or indirectly to a native constitutive pro-protein signal peptide or a synthetic signal peptide as disclosed herein. In some embodiments, the synthetic pre-protein signal peptide is further fused to a native constitutive pro-protein signal peptide. In some embodiments, the synthetic pre-protein signal peptide is further fused to a synthetic signal peptide as disclosed herein.

In some embodiments, a recombinant polypeptide comprising a synthetic pre-protein signal peptide comprising an amino acid sequence of Formula IX or SEQ ID NO. 31, 32, or 33 and a payload protein is provided. In some embodiments, inclusion of the pre-protein signal peptide comprising an amino acid sequence of Formula IX or SEQ ID NO. 31, 32, or 33 will result in the payload protein being more readily secreted by the yeast in which it is produced. Accordingly, in another embodiment, a method of producing a payload protein with Trichoderma yeast (e.g., T. reesei or T. viride) is provided, the method comprising providing a nucleic acid molecule encoding a recombinant polypeptide comprising a payload protein and a synthetic pre-protein signal peptide comprising an amino acid sequence of Formula IX or SEQ ID NO. 31, 32, or 33; genetically modifying the T. reesei yeast with the nucleic acid molecule, thereby generating engineered yeast; and culturing the engineered yeast under effective conditions to express the recombinant polypeptide. In some embodiments, the nucleic acid molecule encoding for the amino acid sequence of Formula IX or SEQ ID NO. 31, 32, or 33 is any nucleic acid molecule encoding for said amino acid sequence.

In some embodiments, a method of increasing extracellular secretion of a payload protein from a Trichoderma yeast (e.g., T. reesei or T. viride) is provided, the method comprising providing a nucleic acid molecule encoding a recombinant polypeptide comprising a payload protein and a synthetic pre-protein signal peptide; genetically modifying the Trichoderma yeast with the nucleic acid, thereby generating an engineered yeast, and culturing the engineered yeast under effective conditions to secrete an increased amount of payload protein when compared to the amount of payload protein secreted by Trichoderma yeast genetically modified to express a recombinant polypeptide comprising the payload protein and pre-protein signal peptide comprising a native pre-protein signal peptide sequence as provided for herein or a control pre-protein signal peptide sequence as provided for herein. In some embodiments, the synthetic pre-protein signal peptide comprises an amino acid sequence of Formula IX or SEQ ID NO. 31, 32, or 33. In some embodiments, the synthetic pre-protein signal peptide further comprises a native pro-protein signal peptide. In some embodiments, the synthetic pre-protein signal peptide further comprises a synthetic pro-protein signal peptide as provided for herein. In some embodiments, the synthetic pre-protein signal peptide is fused directly to the payload protein. In some embodiments, the synthetic pre-protein signal peptide is fused indirectly to the payload protein via, for example, a peptide linker as provided for herein.

In some embodiments, an engineered Trichoderma yeast (e.g., T. reesei or T. viride) is provided, wherein the yeast is genetically modified with a nucleic acid molecule encoding the expression of a recombinant polypeptide comprising a synthetic pre-protein signal peptide fused directly or indirectly to a payload protein. In some embodiments, the synthetic pre-protein signal peptide comprises an amino acid sequence of Formula IX or SEQ ID NO. 31, 32, or 33. In some embodiments, the synthetic pre-protein signal peptide further comprises a native pro-protein signal peptide. In some embodiments, the synthetic pre-protein signal peptide further comprises a synthetic pro-protein signal peptide as provided for herein. In some embodiments, the synthetic pre-protein signal peptide is fused directly to the payload protein. In some embodiments, the synthetic pre-protein signal peptide is fused indirectly to the payload protein via, for example, a peptide linker as provided for herein.

In some embodiments, the payload protein may be any peptide or protein. In some embodiments, the payload protein is selected from the group comprising an enzyme (e.g., invertase, isomaltase, lactase, lysozyme, An-PEP), a growth factor (e.g., IGF-1), insulin, an incretin (e.g., GLP-1, GLP-2, leptin, apelin, ghrelin, PYY, nesfatin), a cytokine, an antibody, an antimicrobial peptide), a mucosal protein (e.g., trefoil factor, Reg3 protein, superoxide dismutase), an agricultural product (e.g., pesticide, bactericide herbicide, fungicide, nematicide, miticide, plant growth regulator, plant growth stimulator, or fertilizer), a vaccine, a diagnostic protein, a feed conversion enzyme, a flavoring, or a nutritional protein. The examples listed are provided for clarity only and are not meant to be limiting in any way. Thus, for example, the current disclosure is not limited to IGF-1 for “growth factor”, but rather encompasses and includes all growth factors known in the art.

Synthetic Pre-Protein Signal Peptides and their Used is Aspergillus Yeast Strains

In some embodiments, a synthetic pre-protein signal peptide for use in the yeast species Aspergillus (e.g., A. niger) is provided. In some embodiments, Aspergillus yeast may be genetically modified with a nucleic acid molecule encoding the expression of a recombinant polypeptide comprising a synthetic pre-protein signal peptide fused directly or indirectly to a payload protein. In some embodiments, the synthetic pre-protein signal peptide comprises an amino acid sequence of Formula XIII or SEQ ID NO. 70, 71, 72, or 73. In some embodiments, the synthetic pre-protein signal peptide is fused directly to the payload protein. In some embodiments, the synthetic pre-protein signal peptide is fused indirectly to the payload protein via, for example, a peptide linker as provided for herein. In some embodiments, any nucleic acid molecule encoding for Formula XIII or SEQ ID NO. 70, 71, 72, or 73 may be utilized to induce expression of the synthetic signal peptide. One of skill in the art will understand how to develop a suitable nucleotide sequence that will induce expression of a synthetic pre-protein signal peptide comprising an amino acid sequence of Formula XIII or SEQ ID NO. 70, 71, 72, or 73. In some embodiments, a synthetic pre-protein signal peptide comprising an amino acid sequence of Formula XIII or SEQ ID NO. 70, 71, 72, or 73 may further be fused directly or indirectly to a native constitutive pro-protein signal peptide or a synthetic signal peptide as disclosed herein. In some embodiments, the synthetic pre-protein signal peptide is further fused to a native constitutive pro-protein signal peptide. In some embodiments, the synthetic pre-protein signal peptide is further fused to a synthetic signal peptide as disclosed herein.

In some embodiments, a recombinant polypeptide comprising a synthetic pre-protein signal peptide comprising an amino acid sequence of Formula XIII or SEQ ID NO. 70, 71, 72, or 73 and a payload protein is provided. In some embodiments, inclusion of the pre-protein signal peptide comprising an amino acid sequence of Formula XIII or SEQ ID NO. 70, 71, 72, or 73 will result in the payload protein being more readily secreted by the yeast in which it is produced. Accordingly, in another embodiment, a method of producing a payload protein with Aspergillus yeast (e.g., A. niger) is provided, the method comprising providing a nucleic acid molecule encoding a recombinant polypeptide comprising a payload protein and a synthetic pre-protein signal peptide comprising an amino acid sequence of Formula XIII or SEQ ID NO. 70, 71, 72, or 73; genetically modifying the Aspergillus yeast with the nucleic acid molecule, thereby generating engineered yeast; and culturing the engineered yeast under effective conditions to express the recombinant polypeptide. In some embodiments, the nucleic acid molecule encoding for the amino acid sequence of Formula XIII or SEQ ID NO. 70, 71, 72, or 73 is any nucleic acid molecule encoding for said amino acid sequence.

In some embodiments, a method of increasing extracellular secretion of a payload protein from a Aspergillus yeast (e.g., A. niger) is provided, the method comprising providing a nucleic acid molecule encoding a recombinant polypeptide comprising a payload protein and a synthetic pre-protein signal peptide; genetically modifying the Aspergillus yeast with the nucleic acid, thereby generating an engineered yeast, and culturing the engineered yeast under effective conditions to secrete an increased amount of payload protein when compared to the amount of payload protein secreted by Aspergillus yeast genetically modified to express a recombinant polypeptide comprising the payload protein and pre-protein signal peptide comprising a native pre-protein signal peptide sequence as provided for herein or a control pre-protein signal peptide. In some embodiments, the control pre-protein signal peptide is:

(SEQ ID NO. 76)
MSFRSLLALSGLVCTGLA

In some embodiments, the control pre-protein signal peptide is glucoamylaseprotein, as represented by SEQ ID NO. 77 below:

(SEQ ID NO. 77)
MSFRSLLALSGLVCTGLANVISKRATLDSWLSNEATVARTAILNNIGADGAWVSGADSGIVVAS
PSTDNPDYFYTWTRDSGLVLKTLVDLFRNGDTSLLSTIENYISAQAIVQGISNPSGDLSSGAGL
GEPKFNVDETAYTGSWGRPQRDGPALRATAMIGFGQWLLDNGYTSTATDIVWPLVRNDLSYVAQ
YWNQTGYDLWEEVNGSSFFTIAVQHRALVEGSAFATAVGSSCSWCDSQAPEILCYLQSFWTGSF
ILANFDSSRSGKDANTLLGSIHTFDPEAACDDSTFQPCSPRALANHKEVVDSFRSIYTLNDGLS
DSEAVAVGRYPEDTYYNGNPWFLCTLAAAEQLYDALYQWDKQGSLEVTDVSLDFFKALYSDAAT
GTYSSSSSTYSSIVDAVKTFADGFVSIVETHAASNGSMSEQYDKSDGEQLSARDLTWSYAALLT
ANNRRNSVVPASWGETSASSVPGTCAATSAIGTYSSVTVTSWPSIVATGGTITTATPTGSGSVT
STSKTTATASKTSTSTSSTSCTTPTAVAVTFDLTATTTYGENIYLVGSISQLGDWETSDGIALS
ADKYTSSDPLWYVTVTLPAGESFEYKFIRIESDDSVEWESDPNREYTVPQACGTSTATVTDTWR

In some embodiments, the synthetic pre-protein signal peptide comprises an amino acid sequence of Formula XIII or SEQ ID NO. 70, 71, 72, or 73. In some embodiments, the synthetic pre-protein signal peptide further comprises a native pro-protein signal peptide. In some embodiments, the synthetic pre-protein signal peptide further comprises a synthetic pro-protein signal peptide as provided for herein. In some embodiments, the synthetic pre-protein signal peptide is fused directly to the payload protein. In some embodiments, the synthetic pre-protein signal peptide is fused indirectly to the payload protein via, for example, a peptide linker as provided for herein.

In some embodiments, an engineered Aspergillus yeast (e.g., A. niger) is provided, wherein the yeast is genetically modified with a nucleic acid molecule encoding the expression of a recombinant polypeptide comprising a synthetic pre-protein signal peptide fused directly or indirectly to a payload protein. In some embodiments, the synthetic pre-protein signal peptide comprises an amino acid sequence of Formula XIII or SEQ ID NO. 70, 71, 72, or 73. In some embodiments, the synthetic pre-protein signal peptide further comprises a native pro-protein signal peptide. In some embodiments, the synthetic pre-protein signal peptide further comprises a synthetic pro-protein signal peptide as provided for herein. In some embodiments, the synthetic pre-protein signal peptide is fused directly to the payload protein. In some embodiments, the synthetic pre-protein signal peptide is fused indirectly to the payload protein via, for example, a peptide linker as provided for herein.

In some embodiments, the payload protein may be any peptide or protein. In some embodiments, the payload protein is selected from the group comprising an enzyme (e.g., invertase, isomaltase, lactase, lysozyme, An-PEP), a growth factor (e.g., IGF-1), insulin, an incretin (e.g., GLP-1, GLP-2, leptin, apelin, ghrelin, PYY, nesfatin), a cytokine, an antibody, an antimicrobial peptide), a mucosal protein (e.g., trefoil factor, Reg3 protein, superoxide dismutase), an agricultural product (e.g., pesticide, bactericide herbicide, fungicide, nematicide, miticide, plant growth regulator, plant growth stimulator, or fertilizer), a vaccine, a diagnostic protein, a feed conversion enzyme, a flavoring, or a nutritional protein. The examples listed are provided for clarity only and are not meant to be limiting in any way. Thus, for example, the current disclosure is not limited to IGF-1 for “growth factor”, but rather encompasses and includes all growth factors known in the art.

### Synthetic Pro-Protein Signal Peptides in Saccharomyces, Pichia, and Kluyveromyces Yeast Strains

In some embodiments, various synthetic pro-protein signal peptides are provided that, in addition to suitability for use in combination with a pre-protein signal peptide as described above, may also be used without a synthetic pre-protein signal peptide. In some embodiments, a pro-protein signal peptide may comprise an amino acid sequence of Formula VI, Formula VII, Formula VIII, or SEQ ID NO. 17, 18, 19, 20, 21, 22, 23, or 24, any of which may be used in any yeast strain as provided for herein, such as Saccharomyces (e.g., S. cerevisiae, S. boulardii), Pichia (e.g., P. pastoris), and/or Kluyveromyces (e.g., K. lactis). In some embodiments, a synthetic signal peptide may comprise only a pro-protein signal peptide comprising an amino acid sequence of Formula VI, Formula VII, Formula VIII, or SEQ ID NO. 17, 18, 19, 20, 21, 22, 23, or 24. In some embodiments, a synthetic signal peptide may further comprise any native constitutive pre-protein signal peptide. In some embodiments, a synthetic signal peptide may further comprise any synthetic pre-protein signal peptides as described herein. In some embodiments, when used in combination with a pre-protein signal peptide (native or synthetic), the N-terminus of the pro-protein signal peptide may be fused directly or indirectly to the C-terminus of the pre-protein signal peptide. The pro-protein signal peptide may, in turn, may be fused directly or indirectly to the N-terminus of a payload protein, optionally through a KR site, Ste13 cleavage site, and/or spacer. In some embodiments, indirect fusion may be accomplished through, for example, inclusion of a linker peptide as provided for herein.

Accordingly, in some embodiments, a synthetic signal peptide is provided, the peptide comprising a pro-protein signal peptide comprising an amino acid sequence of Formula VI, Formula VII, Formula VIII, or SEQ ID NO. 17, 18, 19, 20, 21, 22, 23, or 24 fused directly or indirectly to a payload protein. In some embodiments, the synthetic signal peptide further comprises a pre-protein signal peptide. In some embodiments, the pre-protein signal peptide is a native signal peptide. In some embodiments, the pre-protein signal peptide is a synthetic signal peptide. In some embodiments, the pre-protein signal peptide comprises an amino acid sequence of Formula III, Formula IV, Formula V, or SEQ ID NO. 8, 9, 10, 11, 12, 13, 14, 15, or 16.

In some embodiments, a recombinant polypeptide comprising a synthetic pro-protein signal peptide comprising an amino acid sequence of Formula VI, Formula VII, Formula VIII, or SEQ ID NO. 17, 18, 19, 20, 21, 22, 23, or 24 and a payload protein is provided. In some embodiments, inclusion of the pro-protein signal peptide comprising an amino acid sequence of Formula VI, Formula VII, Formula VIII or SEQ ID NO. 17, 18, 19, 20, 21, 22, 23, or 24 will result in the payload protein being more readily secreted by the yeast in which it is produced. Accordingly, in another embodiment, a method of producing a payload protein with a yeast strain is provided, the method comprising providing a nucleic acid molecule encoding a recombinant polypeptide comprising a payload protein and a synthetic signal peptide comprising an amino acid sequence of Formula VI, Formula VII, Formula VIII or SEQ ID NO. 17, 18, 19, 20, 21, 22, 23, or 24; genetically modifying the yeast with the nucleic acid, thereby generating engineered yeast; and culturing the engineered yeast under effective conditions to express the recombinant polypeptide. In some embodiments, the yeast strain is selected from the group comprising Saccharomyces (e.g., S. cerevisiae, S. boulardii), Pichia (e.g., P. pastoris), and/or Kluyveromyces (e.g., K. lactis). In some embodiments, the nucleic acid molecule encoding for the amino acid sequence of Formula VI, Formula VII, Formula VIII or SEQ ID NO. 17, 18, 19, 20, 21, 22, 23, or 24 is any nucleic acid molecule encoding for said amino acid sequence.

In some embodiments, a method of increasing extracellular secretion of a payload protein from a yeast strain is provided, the method comprising providing a nucleic acid molecule encoding a recombinant polypeptide comprising a payload protein and a synthetic pro-protein signal peptide; genetically modifying the yeast with the nucleic acid molecule, thereby generating an engineered yeast, and culturing the engineered yeast under effective conditions to secrete an increased amount of payload protein when compared to the amount of payload protein secreted by the yeast genetically modified to express a recombinant polypeptide comprising the payload protein and a native pro-protein signal peptide. In some embodiments, the yeast strain is selected from the group comprising Saccharomyces (e.g., S. cerevisiae, S. boulardii), Pichia (e.g., P. pastoris), and/or Kluyveromyces (e.g., K. lactis). In some embodiments, the synthetic pro-protein signal peptide comprises an amino acid sequence of Formula VI, Formula VII, Formula VIII, or SEQ ID NO. 17, 18, 19, 20, 21, 22, 23, or 24. In some embodiments, the synthetic pro-protein further comprises a native pre-protein signal peptide. In some embodiments, the synthetic pro-protein further comprises a synthetic pre-protein signal peptide as provided for herein. In some embodiments, the synthetic pro-protein signal peptide is fused directly to the payload protein. In some embodiments, the synthetic pro-protein signal peptide is fused indirectly to the payload protein via, for example, a peptide linker as provided for herein.

In some embodiments, the payload protein may be any peptide or protein. In some embodiments, the payload protein is selected from the group comprising an enzyme (e.g., invertase, isomaltase, lactase, lysozyme, An-PEP), a growth factor (e.g., IGF-1), insulin, an incretin (e.g., GLP-1, GLP-2, leptin, apelin, ghrelin, PYY, nesfatin), a cytokine, an antibody, an antimicrobial peptide), a mucosal protein (e.g., trefoil factor, Reg3 protein, superoxide dismutase), an agricultural product (e.g., pesticide, bactericide herbicide, fungicide, nematicide, miticide, plant growth regulator, plant growth stimulator, or fertilizer), a vaccine, a diagnostic protein, a feed conversion enzyme, a flavoring, or a nutritional protein. The examples listed are provided for clarity only and are not meant to be limiting in any way. Thus, for example, the current disclosure is not limited to IGF-1 for “growth factor”, but rather encompasses and includes all growth factors known in the art.

### Synthetic Pro-Protein Signal Peptides in Trichoderma Yeast Strains

In some embodiments, various synthetic pro-protein signal peptides are provided that, in addition to suitability for use in combination with a pre-protein signal peptide as described above, may also be used without a synthetic pre-protein signal peptide. In some embodiments, a pro-protein signal peptide may comprise an amino acid sequence of Formula X, Formula XI, or SEQ ID NO. 34, 35, 36, 37, or 38, any of which may be used in any yeast species within the Trichoderma strain (e.g., T. reesei, T. viride). In some embodiments, a synthetic signal peptide may comprise only an amino acid sequence of Formula X, Formula XI, or SEQ ID NO. 34, 35, 36, 37, or 38. In some embodiments, the synthetic signal peptide may further comprise any native constitutive pre-protein signal peptide. In some embodiments, the synthetic signal peptide may further comprise any of the synthetic pre-protein signal peptides as provided for herein. In some embodiments, when used in combination with a pre-protein signal peptide (native or synthetic), the N-terminus of the pro-protein signal peptide may be fused directly or indirectly to the C-terminus of the pre-protein signal peptide. The pro-protein signal peptide may, in turn, may be fused directly or indirectly to the N-terminus of a payload protein, optionally through a KR site, Ste13 cleavage site, and/or spacer. In some embodiments, indirect fusion may be accomplished through, for example, inclusion of a linker peptide as provided for herein.

Accordingly, in some embodiments, a synthetic signal peptide is provided, the synthetic signal peptide comprising a pro-protein signal peptide comprising an amino acid sequence of Formula X, Formula XI, or SEQ ID NO. 34, 35, 36, 37, or 38 fused directly or indirectly to a payload protein. In some embodiments, the synthetic signal peptide further comprises a pre-protein signal peptide. In some embodiments, the pre-protein signal peptide is a native pre-protein signal peptide. In some embodiments, the pre-protein signal peptide is a synthetic pre-protein signal peptide as provided for herein. In some embodiments, the pre-protein signal peptide comprises an amino acid sequence of Formula IX or SEQ ID NO. 31, 32, or 33.

In some embodiments, a recombinant polypeptide comprising a synthetic pro-protein signal peptide comprising an amino acid sequence of Formula X, Formula XI, or SEQ ID NO. 34, 35, 36, 37, or 38 and a payload protein is provided. In some embodiments, inclusion of the pro-protein signal peptide comprising an amino acid sequence of Formula X, Formula XI, or SEQ ID NO. 34, 35, 36, 37, or 38 will result in the payload protein being more readily secreted by the yeast in which it is produced. Accordingly, in another embodiment, a method of producing a payload protein with a yeast strain is provided, the method comprising providing a nucleic acid molecule encoding a recombinant polypeptide comprising a payload protein and a synthetic signal peptide comprising an amino acid sequence of Formula X, Formula XI, or SEQ ID NO. 34, 35, 36, 37, or 38; genetically modifying the yeast with the nucleic acid, thereby generating engineered yeast; and culturing the engineered yeast under effective conditions to express the recombinant polypeptide. In some embodiments, the yeast strain is a Trichoderma yeast strain (e.g., T. reesei, T. viride). In some embodiments, the nucleic acid molecule encoding for the amino acid sequence of Formula X, Formula XI, or SEQ ID NO. 34, 35, 36, 37, or 38 is any nucleic acid molecule encoding for said amino acid sequence.

In some embodiments, a method of increasing extracellular secretion of a payload protein from a yeast strain is provided, the method comprising providing a nucleic acid molecule encoding a recombinant polypeptide comprising a payload protein and a synthetic pro-protein signal peptide; genetically modifying the yeast with the nucleic acid molecule, thereby generating an engineered yeast, and culturing the engineered yeast under effective conditions to secrete an increased amount of payload protein when compared to the amount of payload protein secreted by the yeast genetically modified to express a recombinant polypeptide comprising the payload protein and a native pro-protein signal peptide. In some embodiments, the yeast strain is a Trichoderma yeast strain (e.g., T. reesei, T. viride). In some embodiments, the synthetic pro-protein signal peptide comprises an amino acid sequence of Formula X, Formula XI, or SEQ ID NO. 34, 35, 36, 37, or 38. In some embodiments, the synthetic pro-protein signal peptide further comprises a native pre-protein signal peptide. In some embodiments, the synthetic pro-protein signal peptide further comprises a synthetic pre-protein signal peptide as provided for herein. In some embodiments, the synthetic pro-protein signal peptide is fused directly to the payload protein. In some embodiments, the synthetic pro-protein signal peptide is fused indirectly to the payload protein via, for example, a peptide linker as provided for herein.

In some embodiments, the payload protein may be any peptide or protein. In some embodiments, the payload protein is selected from the group comprising an enzyme (e.g., invertase, isomaltase, lactase, lysozyme, An-PEP), a growth factor (e.g., IGF-1), insulin, an incretin (e.g., GLP-1, GLP-2, leptin, apelin, ghrelin, PYY, nesfatin), a cytokine, an antibody, an antimicrobial peptide), a mucosal protein (e.g., trefoil factor, Reg3 protein, superoxide dismutase), an agricultural product (e.g., pesticide, bactericide herbicide, fungicide, nematicide, miticide, plant growth regulator, plant growth stimulator, or fertilizer), a vaccine, a diagnostic protein, a feed conversion enzyme, a flavoring, or a nutritional protein. The examples listed are provided for clarity only and are not meant to be limiting in any way. Thus, for example, the current disclosure is not limited to IGF-1 for “growth factor”, but rather encompasses and includes all growth factors known in the art.

Synthetic Pro-Protein Signal Peptides and their Use in Aspergillus Yeast Strains

In some embodiments, various synthetic pro-protein signal peptides are provided that, in addition to suitability for use in combination with a pre-protein signal peptide as described above, may also be used without a synthetic pre-protein signal peptide. In some embodiments, a pro-protein signal peptide may comprise an amino acid sequence of Formula XIV, Formula XV, or SEQ ID NO. 74 or 75, any of which may be used in any yeast species within the Aspergillus strain (e.g., A. niger). In some embodiments, a synthetic signal peptide may comprise only an amino acid sequence of Formula XIV, Formula XV, or SEQ ID NO. 74 or 75. In some embodiments, the synthetic signal peptide may further comprise any native constitutive pre-protein signal peptide. In some embodiments, the synthetic signal peptide may further comprise any of the synthetic pre-protein signal peptides as provided for herein. In some embodiments, when used in combination with a pre-protein signal peptide (native or synthetic), the N-terminus of the pro-protein signal peptide may be fused directly or indirectly to the C-terminus of the pre-protein signal peptide. The pro-protein signal peptide may, in turn, may be fused directly or indirectly to the N-terminus of a payload protein, optionally through a KR site, Ste13 cleavage site, and/or spacer. In some embodiments, indirect fusion may be accomplished through, for example, inclusion of a linker peptide as provided for herein.

Accordingly, in some embodiments, a synthetic signal peptide is provided, the synthetic signal peptide comprising a pro-protein signal peptide comprising an amino acid sequence of Formula XIV, Formula XV, or SEQ ID NO. 74 or 75 fused directly or indirectly to a payload protein. In some embodiments, the synthetic signal peptide further comprises a pre-protein signal peptide. In some embodiments, the pre-protein signal peptide is a native pre-protein signal peptide. In some embodiments, the pre-protein signal peptide is a synthetic pre-protein signal peptide as provided for herein. In some embodiments, the pre-protein signal peptide comprises an amino acid sequence of Formula XIII or SEQ ID NO. 70, 71, 72, or 73.

In some embodiments, a recombinant polypeptide comprising a synthetic pro-protein signal peptide comprising an amino acid sequence of Formula XIV, Formula XV, or SEQ ID NO. 74 or 75 and a payload protein is provided. In some embodiments, inclusion of the pro-protein signal peptide comprising an amino acid sequence of Formula XIV, Formula XV, or SEQ ID NO. 74 or 75 will result in the payload protein being more readily secreted by the yeast in which it is produced. Accordingly, in another embodiment, a method of producing a payload protein with a yeast strain is provided, the method comprising providing a nucleic acid molecule encoding a recombinant polypeptide comprising a payload protein and a synthetic signal peptide comprising an amino acid sequence of Formula XIV, Formula XV, or SEQ ID NO. 74 or 75; genetically modifying the yeast with the nucleic acid, thereby generating engineered yeast; and culturing the engineered yeast under effective conditions to express the recombinant polypeptide. In some embodiments, the yeast strain is an Aspergillus yeast strain (e.g., A. niger). In some embodiments, the nucleic acid molecule encoding for the amino acid sequence of Formula XIV, Formula XV, or SEQ ID NO. 74 or 75 is any nucleic acid molecule encoding for said amino acid sequence.

In some embodiments, a method of increasing extracellular secretion of a payload protein from a yeast strain is provided, the method comprising providing a nucleic acid molecule encoding a recombinant polypeptide comprising a payload protein and a synthetic pro-protein signal peptide; genetically modifying the yeast with the nucleic acid molecule, thereby generating an engineered yeast, and culturing the engineered yeast under effective conditions to secrete an increased amount of payload protein when compared to the amount of payload protein secreted by the yeast genetically modified to express a recombinant polypeptide comprising the payload protein and a native pro-protein signal peptide. In some embodiments, the yeast strain is a Aspergillus yeast strain (e.g., A. niger). In some embodiments, the synthetic pro-protein signal peptide comprises an amino acid sequence of Formula XIV, Formula XV, or SEQ ID NO. 74 or 75. In some embodiments, the synthetic pro-protein signal peptide further comprises a native pre-protein signal peptide. In some embodiments, the synthetic pro-protein signal peptide further comprises a synthetic pre-protein signal peptide as provided for herein. In some embodiments, the synthetic pro-protein signal peptide is fused directly to the payload protein. In some embodiments, the synthetic pro-protein signal peptide is fused indirectly to the payload protein via, for example, a peptide linker as provided for herein.

In some embodiments, the payload protein may be any peptide or protein. In some embodiments, the payload protein is selected from the group comprising an enzyme (e.g., invertase, isomaltase, lactase, lysozyme, An-PEP), a growth factor (e.g., IGF-1), insulin, an incretin (e.g., GLP-1, GLP-2, leptin, apelin, ghrelin, PYY, nesfatin), a cytokine, an antibody, an antimicrobial peptide), a mucosal protein (e.g., trefoil factor, Reg3 protein, superoxide dismutase), an agricultural product (e.g., pesticide, bactericide herbicide, fungicide, nematicide, miticide, plant growth regulator, plant growth stimulator, or fertilizer), a vaccine, a diagnostic protein, a feed conversion enzyme, a flavoring, or a nutritional protein. The examples listed are provided for clarity only and are not meant to be limiting in any way. Thus, for example, the current disclosure is not limited to 8GF-1 for “growth factor”, but rather encompasses and includes all growth factors known in the art.

### Methods of Generating Engineered Yeast

Provided herein are synthetic signal peptides that may be used to genetically modify a particular strain of yeast to increase secretion of any payload protein or peptide in that yeast. Various suitable signal peptides are disclosed above with specific examples of signal peptides comprising various synthetic pre- and synthetic pro-protein signal detailed in Table 17 below.

TABLE 17

| Pre-Protein | Pro-Protein | Suitable |
|---|---|---|
| SEQ ID NO. | SEQ ID NO. | Strain/s |
| 1 | 20, 21, 22, 23, 24 | Kluyveromyces |
| 2 (pre-α-MF) | 17, 20, 21, 22, 23, 24 | Pichia, |
| | | Saccharomyces |
| 3 | 20, 21, 22, 23, 24 | Pichia |
| 4 | 20, 21, 22, 23, 24 | Pichia |
| 5 | 20, 21, 22, 23, 24 | Pichia |
| 6 | 20, 21, 22, 23, 24 | Pichia |
| 7 | 20, 21, 22, 23, 24 | Pichia |
| 8 | 18, 20, 21, 22, 23, 24 | Saccharomyces |
| 9 | 19 (TA57), 20, 21, | Saccharomyces |
| | 22, 23, 24 | |
| 10 | 20, 21, 22, 23, 24, 25 | Saccharomyces |
| 11 | 19 (TA57), 20, 21, | Saccharomyces |
| | 22, 23, 24, 25 | |
| 12 | 20, 21, 22, 23, 24, 25 | Saccharomyces |
| 13 | 20, 21, 22, 23, 24, 25 | Saccharomyces |
| 14 | 20, 21, 22, 23, 24, 25 | Saccharomyces |
| 15 | 20, 21, 22, 23, 24, 25 | Saccharomyces |
| 16 | 20, 21, 22, 23, 24, 25 | Saccharomyces |
| 31 | 34, 35, 36, 37, 38 | Trichoderma |
| 32 | 34, 35, 36, 37, 38 | Trichoderma |
| 33 | 34, 35, 36, 37, 38 | Trichoderma |
| 2 (pre-α-MF) | 17 | Pichia |
| 8 | 18 | Saccharomyces |
| 9, 11 | 19 (TA57) | Saccharomyces |
| 1 | 20 | Kluyveromyces |
| 2 (pre-α-MF), 3, 4, 5, 6, 7 | 20 | Pichia |
| 2 (pre-α-MF), 8, 9, 10, | 20 | Saccharomyces |
| 11, 12, 13, 14, 15, 16 | | |
| 1 | 21 | Kluyveromyces |
| 2 (pre-α-MF), 3, 4, 5, 6, 7 | 21 | Pichia |
| 2 (pre-α-MF), 8, 9, 10, | 21 | Saccharomyces |
| 11, 12, 13, 14, 15, 16 | | |
| 2 (pre-α-MF), 8, 9, 10, | 22 | Saccharomyces |
| 11, 12, 13, 14, 15, 16 | | |
| 2 (pre-α-MF), 8, 9, 10, | 23 | Saccharomyces |
| 11, 12, 13, 14, 15, 16 | | |
| 2 (pre-α-MF), 8, 9, 10, | 24 | Saccharomyces |
| 11, 12, 13, 14, 15, 16 | | |
| 2 | 25 | Pichia, |
| | | Saccharomyces |
| 9 | 25 | Saccharomyces |
| 31, 32, 33 | 34 | Trichoderma |
| 31, 32, 33 | 35 | Trichoderma |
| 31, 32, 33 | 36 | Trichoderma |
| 31, 32, 33 | 37 | Trichoderma |
| 31, 32, 33 | 38 | Trichoderma |
| 8, 9, 10, 11, 12, 13, | 27 | Saccharomyces |
| 14, 15, 16 | | |
| 28 (pre-α-MF) | 20, 21 | Kluyveromyces |
| 3, 4, 5, 6, 7 | 29 | Pichia |
| 1 | 29 | Kluyveromyces |
| 70, 71, 72, 73 | 74,75 | Aspergillus |

The suitable strains recited in the prior table are meant to be exemplary, not exclusionary Thus, the table should not be interpreted as suggesting that the “suitable strains” are the only strains for which the recited pre and pro protein signal peptides can be used. Rather, the “suitable strain” is merely an example of a strain in which the recited pre and pro protein signal peptides can be used.

In some embodiments, any synthetic signal sequence may comprise solely a synthetic pre-protein signal peptide (e.g., SEQ ID NOs. 1, 2, 3, 4, 5, 6, 7, 8, 9, 10, 11, 12, 13, 14, 15, 16, 28, 31, 32, 33, 55, 70, 71, 72, or 73) with no additional pro-protein signal peptide sequence. In some embodiments, any synthetic signal sequence may comprise a pre-protein signal peptide (e.g., SEQ ID NOs. 1, 2, 3, 4, 5, 6, 7, 8, 9, 10, 11, 12, 13, 14, 15, 16, 28, 31, 32, 33, 55, 70, 71, 72, or 73) fused to any native pro-protein peptide or portion thereof (e.g., pro-α-MF). In some embodiments, any synthetic signal sequence may comprise a pre-protein signal peptide (e.g., SEQ ID NOs. 1, 2, 3, 4, 5, 6, 7, 8, 9, 10, 11, 12, 13, 14, 15, 16, 28, 31, 32, 33, 55, 70, 71, 72, or 73) fused to any synthetic pro-protein signal peptide (e.g., SEQ ID NOs 17, 18, 19, 20, 21, 22, 23, 24, 25, 27, 29, 34, 35, 36, 37, 38, 56, 57, 58, 74, or 75) or portion thereof. In some embodiments, any synthetic signal sequence may comprise solely a synthetic pro-protein signal peptide (e.g., SEQ ID NOs 17, 18, 19, 20, 21, 22, 23, 24, 25, 27, 29, 34, 35, 36, 37, 38, 56, 57, 58, 74, or 75) with no additional pre-protein signal peptide sequence. In some embodiments, any synthetic signal peptide may comprise a pro-protein signal peptide (e.g., SEQ ID NOs. 17, 18, 19, 20, 21, 22, 23, 24, 25, 27, 29, 34, 35, 36, 37, 38, 56, 57, 58, 74, or 75) fused to any native pre-protein signal peptide or portion thereof (e.g., pre-α-MF, SUC2 pre). In some embodiments, any synthetic signal sequence may comprise a pro-protein signal peptide (e.g., SEQ ID NOs. 17, 18, 19, 20, 21, 22, 23, 24, 25, 27, 29, 34, 35, 36, 37, 38, 56, 57, 58, 74, or 75) fused to any synthetic pre-protein signal peptide (e.g., SEQ ID NO.s. 1, 2, 3, 4, 5, 6, 7, 8, 9, 10, 11, 12, 13, 14, 15, 16, 28, 31, 32, 33, 55, 70, 71, 72, or 73) or portion thereof. Other examples of signal peptides that may be incorporated in their entirety or in part into a synthetic signaling peptide include but are not limited to, HSp150, PHO1, PHO5, SUC2, KILM1, GGP1, SUN, PLB, CRH, EXG, AGA2, HAS pre-pro, PIR1, XPR2 pre, XPR2 pre-pro, pGKL, SCW, and DSE.

In some embodiments, a method of generating an engineered yeast that expresses a recombinant polypeptide comprising a synthetic signal peptide is provided, the method comprising providing a yeast, contacting the yeast with a nucleic acid molecule encoding the recombinant polypeptide comprising the synthetic signal peptide, and culturing the yeast under conditions suitable to genetically modify the yeast to induce expression of the recombinant polypeptide, thereby creating an engineered yeast.

The yeast may be any strain of yeast, such as, but not limited to, Kluyveromyces (e.g., K. lactis), Pichia (e.g., P. pastoris), Saccharomyces (e.g., S. cerevisiae, S. boulardii), Trichoderma (e.g., T. reesei, T. viride), and Aspergillus (e.g., A. niger). In some embodiments, inducing expression of the recombinant polypeptide may be carried out via any expression system known to those skilled in the art. For example, in some embodiments, the method of generating an engineered yeast may comprise preparing a vector containing a nucleic acid (e.g., RNA, DNA) encoding the recombinant polypeptide, transporting the vector to the host yeast (“genetically modifying”), and culturing the yeast under effective conditions to express the recombinant polypeptide. As used herein, the term “vector” refers to a nucleotide molecule capable of transporting other nucleotides to which it has been linked. One exemplary type of vector is a “plasmid”, which represents a circular double stranded DNA loop into which additional DNA sections can be ligated. Another type of vector is a viral vector; wherein additional DNA sections can be ligated with the viral genome. Methods of introducing a DNA into yeast are known to those skilled in the art and may include a transformation method, a transfection method, an electroporation method, a nuclear injection method, or a carrier such as a liposome, micelle, skin cell, or a fusion method using protoplasts. A recombinant nucleic acid encoding the recombinant polypeptide may be obtained from any source using conventional techniques known to those skilled in the art, including isolation from genomic or cDNA libraries, amplification by PCR, or chemical synthesis.

In some embodiments, an engineered yeast may be cultured to induce growth of the yeast for a period of time in an environment effective to maintain the health of the yeast, thereby generating a desired amount of recombinant polypeptide comprising the synthetic signal peptide and payload protein. The culturing of yeast is common practice and well known in the art. In general, yeast can be grown in broth or agar in the presence of culture medium comprising bacteriological peptone, yeast extract, and glucose. Supplemental components such as amino acids, buffers, polysaccharides, and salts are sometimes used as well, depending on the strain and application. Engineered yeast may be grown at room temperature or, more effectively, at a temperature of up to about 30° C. to 37° C. Temperature may be used to control the growth of the yeast cells and to regulate the production of the desired recombinant polypeptide. Thus, in some embodiments, the yeast may be grown at a temperature from about 4° C. to about 50° C. The recited temperature range includes any temperature range within said range. Thus, in some embodiments, the yeast may be grown at a temperature from about 4° C. to about 40° C., from about 10° C. to about 50° C., from about 10° C., to about 45° C., from about 15° C., to about 45° C., from about 20° C. to about 45° C., from about 25° C. to about 45° C., from about 30° C. to about 50° C., from about 35° C. to about 50° C., from about 37° C. to about 50° C., from about 40° C. to about 50° C., or from about 45° C. to about 50° C. Similarly, the recited ranges include each and every individual temperature within said range. Thus, in some embodiments, the yeast may be grown at a temperature of about 4° C. In some embodiments, the yeast may be grown at a temperature of about 50° C. In some embodiments, the yeast may be grown at a temperature of about 4° C., about 5° C., about 6° C., about 7° C., about 8° C., about 9° C., about 10° C., about 11° C., about 12° C., about 13° C., about 14° C., about 15° C., about 16° C., about 17° C., about 18° C., about 19° C., about 20° C., about 21° C., about 22° C., about 23° C., about 24° C., about 25° C., about 26° C., about 27° C., about 28° C., about 29° C., about 30° C., about 31° C., about 32° C., about 33° C., about 34° C., about 35° C., about 36° C., about 37° C., about 38° C., about 39° C., about 40° C., about 41° C., about 42° C., about 43° C., about 44° C., about 45° C., about 46° C., about 47° C., about 48° C., about 49° C., or about 50° C. Further, those skilled in the art will recognize that further modifications to the growth conditions may be necessary depending on the strain of yeast utilized and the recombinant polypeptide being produced. Such modifications are within the scope of the present application. In any case, secretion of a payload protein by the host yeast will result in its accumulation in the surrounding culture medium, where it may then be collected, isolated, and/or quantified. Through various intracellular mechanisms, the payload protein will be extracellularly secreted with or without some or all of the synthetic signal peptide to which it was fused.

In some embodiments, the proteins that may be produced by the engineered yeast include any protein. In some embodiments, the proteins that may be produced by the engineered yeast disclosed herein include, but are not limited to, maltose binding protein (MBP), trefoil factor, mucin, DNase, clotting or blood volumizing factors, insulin and insulin analogs, an incretin (e.g., GLP-1, GLP-2, leptin, apelin, ghrelin, PYY, nesfatin), EGFP, PDGF, HB-EGF, α1-antitrypsin, serum albumin, collagen, pepsinogen, tumor necrosis factor, streptokinase, glucagon, lepirudin, desirudin, hirudin, encallantide, IFN-α 2b, antigens and antibodies (e.g., anti-IL-6R Ab, anti-RSV ab, tetanus toxin fragment C, An-PEP, HIV-1 gp120 (intracellular), HIV-1 gp120 (secret), Bm86 tick gut glytoprotein, murine single-chain antibody, anti-TNF Ab, cancer antibodies, sHBsAg), enzymes (e.g., lysozyme, invertase, galactanase, isomaltase, lactase, chitiniase, xylanase, catalase, D-alanine carboxypeptidase, α-amylase, aspartic proteinase II, galactosidase, horseradish peroxidase, rasburicase, ocriplasmin, pancrelipase, alcohol dehydrogenase (I and II), phosphoglyserate kinase, GADPH, acid phosphatase), enzyme inhibitors (e.g., Kunitz protease inhibitor, tick anticoagulant protein, ghilanten, tPA Kringle type-2 domain), hormones (e.g., HGH, follicle stimulating hormone, human parathyroid hormone), vaccines (e.g., hepatitis vaccine (I), HPV vaccine), food processing products (e.g., brazzein, chymocin, beta-galactosidase), and cytokines.

In some embodiments, secretion of a payload protein by a yeast is increased by genetically modifying the yeast to express the payload protein as part of a recombinant polypeptide comprising a synthetic signal peptide as disclosed herein. Accordingly, in some embodiments, an engineered yeast may secrete about 10% to about 200% more of a payload protein than a yeast expressing a native signal peptide. In some embodiments, an engineered yeast may express about 10% to about 50% more, about 20% to about 70% more, about 30% to about 90% more, or about 50% to about 200% more of a payload protein. It is to be understood that any individual percentage of increased payload protein secretion is encompassed within the embodiments described herein. Accordingly, in some embodiments, the yeast may secrete about 10% more of a payload protein. In some embodiments, the yeast may secrete about 20% more of a payload protein. In some embodiments, the yeast may secrete about 10%, about 20%, about 30%, about 40%, about 50%, about 60%, about 70%, about 80%, about 90%, about 100%, about 110%, about 120%, about 130%, about 140%, about 150%, about 160%, about 170%, about 180%, about 190%, or about 200% more of a payload protein, or any percentage falling within any of the recited percentages. Those of skill in the art would recognize that any change in growth condition during routine optimization for expression of a particular recombinant polypeptide of interest may also affect the amount of payload protein secreted by the engineered yeast. Accordingly, in some embodiment, an engineered yeast may secrete at least 10% more of a payload protein. Accordingly, in some embodiments, an engineered yeast may secrete about 10% more, about 100%, about 500% more, about 1000% more, or about 10,000% more of a payload protein compared to a yeast expressing a native signal peptide. In some embodiments, secretion is measured by measuring the concentration of the payload protein in the culture media in which the yeast was grown. The concentration may be normalized to optical density to account for variations in growth of the yeast. In some embodiments, secretion is measured by any method known to those skilled in the art for measuring payload protein concentration.

In some embodiments, the payload protein may be isolated from the culture medium in which the engineered yeast is grown using any methods known to those skilled in the art, such as precipitation from the medium, immunoaffinity chromatography, receptor affinity chromatography, or hydrophobic interaction chromatography. In some embodiments, the payload protein may be isolated by conventional chromatographic methods such as affinity chromatography, size-exclusion filtration, cation or anion exchange chromatography, high pressure liquid chromatography (HPLC), reverse phase HPLC, and the like.

In some embodiments, a recombinant polypeptide may be designed to comprise a specific affinity peptide, tag, label, or chelate residue that is recognized by a specific binding partner or agent which may aid in isolation. In some embodiments, the recombinant polypeptide variants comprising the additional tag, label, or residue may then be cleaved to obtain the payload protein.

Synthetic Signal Peptides and Methods of their Use

In some embodiments, the various signal peptides disclosed herein may be utilized in yeast to deliver any payload protein to any environment. In some embodiments, an engineered yeast utilizing a signal peptide as disclosed herein may be used to deliver one or more of a therapeutic protein, diagnostic protein, or protein-based vaccine to a subject in need thereof. In some embodiments, the engineered yeast utilizing a signal peptide as disclosed herein may be used to deliver a payload protein to a specific organ or location within the subject, for example, to a subject's GI tract, skin, reproductive tract, or the like. In some embodiments the subject may be an animal, such as a companion animal (e.g., dog, cat, rodent, or the like). In some embodiments, the subject may be a livestock animal (e.g., cattle, sheep, horse, pig, goat, or the like). In some embodiments, the subject is a human.

In some embodiments, an engineered yeast may be used to deliver one or more of a protein-based herbicide, fungicide, bactericide, insecticide, nematicide, miticide, plant growth regulator, plant growth stimulant, or fertilizer in an agricultural environment, such as to crops or plants (such as seeds, roots, corn, tubers, bulbs, slip, rhizome, grass, or vines) or to a plant growth environment (such as topsoil, top dressing, compost, manure, water table, or hydroponic tank).

In some embodiments, an engineered yeast may be incorporated into a food product, such as bread, dairy, or fermented beverage, to deliver a therapeutic protein, diagnostic protein, protein-based vaccine, an anti-spoilage agent (e.g., bactericide or fungicide), protein-based flavoring agent, protein supplement, or an allergen degrader (e.g., gluten enzyme).

In some embodiments, an engineered yeast may be used to deliver any protein in any application or environment where fermentation is desired. Further specific uses are described herein below.

Therapeutic Compositions and Methods of their Use

The synthetic signal peptides and methods for their use, as disclosed herein, may be used to facilitate secretion of a payload protein expressed by a yeast. In some embodiments, the payload protein may have therapeutic efficacy and as such, may be used to treat a condition, disorder, or disease in a subject. Accordingly, in some embodiments, a method of treating a condition, disorder, or disease in a subject in need thereof in provided, the method comprising administering a composition comprising a therapeutically effective amount of a protein, wherein the protein is produced in an engineered yeast genetically modified with a nucleic acid encoding a recombinant polypeptide comprising one or both of a synthetic pre-protein signal and a synthetic pro-protein signal as disclosed herein. In some embodiments, administering may be performed via any route, such as oral or topical. In some embodiments, the composition is administered orally. In some embodiments, the composition is administered topically.

In some embodiments, a pharmaceutical composition comprising a therapeutically effective amount of a therapeutic payload protein is provided, wherein the therapeutic payload protein is generated by an engineered yeast genetically modified with a nucleic acid molecule encoding a recombinant polypeptide comprising one or both of a synthetic pre- and pro-protein signal peptide, as disclosed in any aspect or embodiment herein. In some embodiments, the disease or condition may include, but is not limited to, an infection, an autoimmune disease, enzymatic deficiencies (including primary (congenital) enzymatic deficiency and enzymatic deficiencies secondary to functional gut disorders), diabetes, obesity, metabolic disorders, intestinal bacterial overgrowth, enteric infection, bacterial vaginosis, short bowel syndrome, inflammatory bowel disease, irritable bowel syndrome, small bowel syndrome, Celiac disease, gluten intolerance, colitis, peptic ulcer, gastritis, polyps, hemorrhoids, cirrhosis, or a cancer.

In some embodiments, a composition comprising a therapeutic protein that is produced by any engineered yeast disclosed herein may be formulated for oral, topical, parenteral, or transdermal administration. These compositions may be in form of pill, tablet, capsule, microcapsule, powder, sachet, dragee, gel, liquid, suspension, solution, food product, cream or granule, and may further comprise one or more pharmaceutically acceptable excipients such as, but not limited to, carriers, solvents, co-solvents, emulsifiers, lubricants, disintegrants, binders, fillers, glidants, rheology agents, solubilizers, antimicrobials, antioxidants, preservatives, colorants, flavor agents, emollients, pH modifiers, and the like.

In some embodiments, food products may include, but are not limited to, a dairy product, a yoghurt, an ice cream, a milk-based drink, a milk-based garnish, a pudding, a milkshake, an ice tea, a fruit juice, a diet drink, a soda, a sports drink, a powdered drink mixture for dietary supplementation, an infant and baby food, a calcium-supplemented orange juice, a sauce or a soup.

In some embodiments, the engineered yeast may be utilized as a conduit for drug delivery to a subject. For example, engineered yeast may be orally administered to a subject to treat a condition, disorder, or disease, wherein the engineered yeast continues to produce and secrete the therapeutic protein within the subject, therefore providing a therapeutic benefit to the subject. Accordingly, in some embodiments, a method of treating a condition, disorder, or disease in a subject in need thereof is provided, the method comprising administering a therapeutically effective amount of engineered yeast as described herein, to the subject. In some embodiments, the therapeutically effective amount of engineered yeast may be orally administered to the subject. In some embodiments, the condition, disorder, or disease may include, but is not limited to, a GI disease or condition, a topical disease or condition, or a mucosal disease or condition. For example, the disease can be a viral (e.g. rotavirus), bacterial, fungal, or parasitic infection (such as, but not limited to intestinal bacterial overgrowth, bacterial vaginosis, an STI), an autoimmune disease (e.g., GBS), an enzymatic or vitamin deficiency (such as lactose intolerance, CSID, Celiac disease/gluten intolerance), a metabolic disorder such as diabetes, an inflammatory GI disease (e.g., irritable bowel syndrome, inflammatory bowel disease, colitis, gastritis, polyps), other GI condition or disease where healing/repair is required (e.g., peptic ulcer), an inflammatory skin condition (e.g. atopic dermatitis, diabetic ulcer), a wound, short bowel syndrome, hemorrhoids, cirrhosis, or a cancer. In some embodiments, administering may be performed via any route, such as oral or topical. The therapeutically effective amount of engineered yeast may be measured in colony forming units (CFUs) and may be any amount, such as from about 100 CFUs to 10²⁰ CFUs, about 10³ to 10¹⁵ CFUs, 10⁴ to 10¹⁰ CFUs, or about 10² to about 10⁸ CFUs. In some embodiments, the therapeutically effective amount of engineered yeast is from about 100 CFUs to about 10²⁰ CFUs. In some embodiments, the therapeutically effective amount of engineered yeast is from about 10³ to about 10¹⁵ CFUs. In some embodiments, the therapeutically effective amount of engineered yeast is from about 100 CFUs, about 10³ CFUs, or about 10⁴ CFUs to about 10⁸ CFUs, about 10¹⁰ CFUs, about 10¹⁵ CFUs, or about 10²⁰ CFUs. In some embodiments, the therapeutically effective amount of engineered yeast is any amount of CFU that falls within any of the above ranges.

In another embodiment, a pharmaceutical composition comprising an engineered yeast genetically modified with a nucleic acid molecule encoding a recombinant polypeptide comprising one or both of a synthetic pre- and pro-protein signal peptide, as disclosed in any aspect or embodiment herein, and a payload protein is provided.

In some embodiments, the composition comprises a Kluyveromyces yeast (e.g., K. lactis) genetically modified with a nucleic acid molecule encoding a recombinant polypeptide comprising one or both of a) a pre-protein signal peptide comprising an amino acid sequence of Formula I or SEQ ID NO. 1 and b) a pro-protein signal peptide comprising an amino acid sequence of Formula VI or SEQ ID NO. 20 or 21 fused directly or indirectly thereto and a payload protein. In some embodiments, the one or both signal peptides are fused directly to the payload protein. In some embodiments, the one or both signal peptides are fused indirectly to the payload protein via, for example, a linker peptide as provided for herein.

In some embodiments, the composition comprises a Pichia yeast (e.g., P. pastoris) genetically modified with a nucleic acid molecule encoding a recombinant polypeptide comprising one or both of a) a pre-protein signal peptide comprising an amino acid sequence of SEQ ID NO. 2, 3, 4, 5, 6, or 7 and b) a pro-protein signal peptide comprising an amino acid sequence of Formula VI or SEQ ID NO. 17, 20, 21 fused directly or indirectly thereto and a payload protein. In some embodiments, the one or both signal peptides are fused directly to the payload protein. In some embodiments, the one or both signal peptides are fused indirectly to the payload protein via, for example, a linker peptide as provided for herein.

In some embodiments, the composition comprises a Saccharomyces yeast (e.g. S. boulardii or S. cerevisiae) genetically modified with a nucleic acid molecule encoding a recombinant polypeptide comprising one or both of a) a pre-protein signal peptide comprising an amino acid sequence of Formula III, Formula IV, Formula V, or SEQ ID NO. 8, 9, 10, 11, 12, 13, 14, 15, or 16 and b) a pro-protein signal peptide comprising an amino acid sequence of Formula VI, Formula VII, Formula VIII, or SEQ ID NO. 17, 18, 19, 20, 21, 22, 23, 24, 25 fused directly or indirectly thereto and a payload protein. In some embodiments, the one or both signal peptides are fused directly to the payload protein. In some embodiments, the one or both signal peptides are fused indirectly to the payload protein via, for example, a linker peptide as provided for herein.

In some embodiments, the composition comprises a Trichoderma yeast (e.g., T. reesei or T. viride) genetically modified with a nucleic acid molecule encoding recombinant polypeptide comprising one or both of a) a pre-protein signal peptide comprising an amino acid sequence of Formula IX or SEQ ID NO. 31, 32, or 33 and b) a pro-protein signal peptide comprising an amino acid sequence of Formula X, Formula XI, or SEQ ID NO. 34, 35, 36, 37, or 38 fused directly or indirectly thereto and a payload protein. In some embodiments, the one or both signal peptides are fused directly to the payload protein. In some embodiments, the one or both signal peptides are fused indirectly to the payload protein via, for example, a linker peptide as provided for herein.

In some embodiments, the composition comprises an Aspergillus yeast (e.g., A. niger) genetically modified with a nucleic acid molecule encoding recombinant polypeptide comprising one or both of a) a pre-protein signal peptide comprising an amino acid sequence of Formula XIII or SEQ ID NO. 70, 71, 72, or 73 and b) a pro-protein signal peptide comprising an amino acid sequence of Formula XIV, Formula XV, or SEQ ID NO. 74 or 75 fused directly or indirectly thereto and a payload protein. In some embodiments, the one or both signal peptides are fused directly to the payload protein. In some embodiments, the one or both signal peptides are fused indirectly to the payload protein via, for example, a linker peptide as provided for herein.

In some embodiments, the disease or condition is an enzyme deficiency, and the payload protein is an enzyme.

In some embodiments, the disease or condition is congenital sucrose-isomaltase deficiency and the payload protein is one or both of invertase and isomaltase.

In some embodiments, the disease or condition is sucrose intolerance secondary to a functional gut disorder and the payload protein is one or both of invertase and isomaltase. In some embodiments, the disease or condition is isomaltase intolerance secondary to a functional gut disorder and the payload protein is one or both of invertase and isomaltase. In some embodiments, the disease or condition is one or both of sucrose and isomaltase intolerance secondary to a functional gut disorder and the payload protein is one or both of invertase and isomaltase.

In some embodiments, the disease or condition is one or more of gluten intolerance, refractory sprue, or Celiac disease and the payload protein is one or more of An-PEP, Mx-PEP, Aspergillus tubigensis prolyl endopeptidase, subtilisin, sedolisin, and larozotide. In some embodiments, the disease or condition is gluten intolerance and the payload protein is one or more of An-PEP, Mx-PEP, Aspergillus tubigensis prolyl endopeptidase, subtilisin, sedolisin, and larozotide. In some embodiments, the disease or condition is refractory sprue and the payload protein is one or more of An-PEP, Mx-PEP, Aspergillus tubigensis prolyl endopeptidase, subtilisin, sedolisin, and larozotide. In some embodiments, the disease or condition is Celiac disease and the payload protein is one or more of An-PEP, Mx-PEP, Aspergillus tubigensis prolyl endopeptidase, subtilisin, sedolisin, and larozotide.

In some embodiments, the disease or condition is pancreatitis or exocrine pancreatic insufficiency and the payload protein is selected from one or more of triacylglycerol lipase, colipase, alpha-amylase, trypsin, and chymotrypsin. In some embodiments, the disease or condition is pancreatitis and the payload protein is selected from one or more of triacylglycerol lipase, colipase, alpha-amylase, trypsin, and chymotrypsin. In some embodiments, the disease or condition is exocrine pancreatic insufficiency and the payload protein is selected from one or more of triacylglycerol lipase, colipase, alpha-amylase, trypsin, and chymotrypsin.

In some embodiments, the disease or condition is enteropeptidase deficiency or enterokinase deficiency and the payload protein is one or all of enteropeptidase, proenteropeptidase, and enterokinase. In some embodiments, the disease or condition is enteropeptidase deficiency and the payload protein is one or all of enteropeptidase, proenteropeptidase, and enterokinase. In some embodiments, the disease or condition is enterokinase deficiency and the payload protein is one or all of enteropeptidase, proenteropeptidase, and enterokinase.

In some embodiments, the disease or condition is small intestinal bacterial overgrowth, inflammatory bowel disease, irritable bowel syndrome, C. difficile infection, cystic fibrosis, necrotizing enterocolitis, and diabetes, and the payload protein is intestinal alkaline phosphatase. In some embodiments, the disease or condition is small intestinal bacterial overgrowth, and the payload protein is intestinal alkaline phosphatase. In some embodiments, the disease or condition is inflammatory bowel disease and the payload protein is intestinal alkaline phosphatase. In some embodiments, the disease or condition is irritable bowel syndrome and the payload protein is intestinal alkaline phosphatase. In some embodiments, the disease or condition is C. difficile infection and the payload protein is intestinal alkaline phosphatase. In some embodiments, the disease or condition is cystic fibrosis and the payload protein is intestinal alkaline phosphatase. In some embodiments, the disease or condition is necrotizing enterocolitis and the payload protein is intestinal alkaline phosphatase. In some embodiments, the disease or condition is diabetes and the payload protein is intestinal alkaline phosphatase.

In some embodiments, the disease or condition is short bowel syndrome and the payload protein is IGF-1, GLP-2, or a synthetic derivative of GLP-2. In some embodiments, the disease or condition is short bowel syndrome and the payload protein is IGF-1. In some embodiments, the disease or condition is short bowel syndrome and the payload protein is GLP-2. In some embodiments, the disease or condition is short bowel syndrome and the payload protein is a synthetic derivative of GLP-2.

In some embodiments, the disease or condition is lactose sensitivity or lactose intolerance and the payload protein is lactase. In some embodiments, the disease or condition is lactose sensitivity and the payload protein is lactase. In some embodiments, the disease or condition is lactose intolerance and the payload protein is lactase.

In some embodiments, the disease or condition is trehalose sensitivity or lactose intolerance and the payload protein is trehalase.

In some embodiments, the disease or condition is maltose sensitivity or lactose intolerance and the payload protein is maltase. In some embodiments, the disease or condition is maltose sensitivity and the payload protein is maltase. In some embodiments, the disease or condition is lactose intolerance and the payload protein is maltase.

In some embodiments, the disease or condition is pernicious anemia and the payload protein is intrinsic factor.

In some embodiments, the disease or condition is bacterial overgrowth and the payload protein is lysozyme, nisin, a defensin, magainin, cateslytin, or any combination thereof. In some embodiments, the disease or condition is bacterial overgrowth and the payload protein is lysozyme. In some embodiments, the disease or condition is bacterial overgrowth and the payload protein is nisin. In some embodiments, the disease or condition is bacterial overgrowth and the payload protein is a defensing. In some embodiments, the disease or condition is bacterial overgrowth and the payload protein is magainin. In some embodiments, the disease or condition is bacterial overgrowth and the payload protein is cateslytin.

In some embodiments, the disease or condition is type 1 or type 2 diabetes mellitus and the payload protein is insulin, or an incretin. In some embodiments, the disease or condition is type 1 diabetes mellitus and the payload protein is insulin, or an incretin. In some embodiments, the disease or condition is type 1 diabetes mellitus and the payload protein is insulin. In some embodiments, the disease or condition is type 1 diabetes mellitus and the payload protein is an incretin. In some embodiments, the disease or condition is type 2 diabetes mellitus and the payload protein is insulin, or an incretin. In some embodiments, the disease or condition is type 2 diabetes mellitus and the payload protein is insulin. In some embodiments, the disease or condition is type 2 diabetes mellitus and the payload protein is an incretin.

In some embodiments, the disease or condition has an inflammatory component and the payload protein is IL-10, IL-22, TGFβ, or any combination thereof.

Methods of Treating Invertase/Sucrase and/or Isomaltase Deficiency

An engineered yeast may be used, for example, to treat an enzyme deficiency such as a deficiency of invertase and/or isomaltase. Accordingly, in some embodiments a method of treating a sucrase/invertase and/or isomaltase deficiency in a subject in need thereof is provided, the method comprising orally administering to the subject one or both of 1) a therapeutically effective amount of an engineered yeast genetically modified to express a first recombinant polypeptide comprising invertase (or a pro-drug or active variant thereof) and a first synthetic signal peptide and 2) a therapeutically effective amount of an engineered yeast genetically modified to express a recombinant polypeptide comprising isomaltase (or a pro-drug or active variant thereof) and a second synthetic signal peptide, thereby treating the invertase and/or isomaltase deficiency. In some embodiments, the first and second synthetic signal peptide independently comprise one or both of a) a pre-protein amino acid sequence of Formula II, Formula III, Formula IV, Formula V, Formula IX, Formula XIII or SEQ ID NO. 1, 2, 3, 4, 5, 6, 7, 8, 9, 10, 11, 12, 13, 14, 15, 16, 28, 31, 32, 33, 55, 70, 71, 72, or 73; and b) a pro-protein amino acid sequence of Formula VI, Formula VII, Formula VIII, Formula X, Formula XI, Formula XIV, Formula XV or SEQ ID NO. 17, 18, 19, 20, 21, 22, 23, 24, 25, 27, 29, 34, 35, 36, 37, 38, 56, 57, 58, 74, or 75. In some embodiments, the engineered yeast may be any strain as disclosed herein. In some embodiments, the engineered yeast is selected from the group comprising Kluyveromyces (e.g., K. lactis), Pichia (e.g., P. pastoris), Saccharomyces (e.g., S. cerevisiae, S. boulardii), Trichoderma (e.g., T. reesei, T. viride), and Aspergillus (e.g., A. niger). In some embodiments, the invertase and/or isomaltase deficiency may be secondary to a functional gut disorder, such as, but not limited to, irritable bowel syndrome, functional dyspepsia, functional vomiting, functional abdominal pain, functional constipation, and/or functional diarrhea.

In some embodiments, a method of treating a sucrase/invertase and/or isomaltase deficiency is provided, the method comprising administering to a subject in need thereof Kluyveromyces yeast (e.g., K. lactis), genetically modified with one or more of 1) a nucleic acid encoding a recombinant polypeptide comprising invertase and one or both of a) a pre-protein signal peptide comprising an amino acid sequence of Formula I or SEQ ID NO. 1 and b) a pro-protein signal peptide comprising an amino acid sequence of Formula VI or SEQ ID NO. 20 or 21 and 2) a nucleic acid encoding a recombinant polypeptide comprising isomaltase and one or both of a) a pre-protein signal peptide comprising an amino acid sequence of Formula I or SEQ ID NO. 1 and b) a pro-protein signal peptide comprising an amino acid sequence of Formula VI or SEQ ID NO. 20 or 21, thereby treating the deficiency.

In some embodiments, a method of treating a sucrase/invertase and/or isomaltase deficiency is provided, the method comprising administering to a subject in need thereof Pichia yeast (e.g., P. pastoris), genetically modified with one or both of 1) a nucleic acid encoding a recombinant polypeptide comprising invertase and one or both of a) a pre-protein signal peptide comprising an amino acid sequence of Formula II or SEQ ID NO. 2, 3, 4, 5, 6, or 7 and b) a pro-protein signal peptide comprising an amino acid sequence of Formula VI or SEQ ID NO. 17, 20 or 21 and 2) a nucleic acid encoding a recombinant polypeptide comprising isomaltase and one or both of a) a pre-protein signal peptide comprising an amino acid sequence of SEQ ID NO. 2, 3, 4, 5, 6, or 7 and b) a pro-protein signal peptide comprising an amino acid sequence of Formula VI or SEQ ID NO. 17, 20 or 21, thereby treating the deficiency.

In some embodiments, a method of treating a sucrase/invertase and/or isomaltase deficiency is provided, the method comprising administering to a subject in need thereof a Saccharomyces yeast (e.g., S. cerevisiae or S. boulardii), genetically modified with one or both of 1) a nucleic acid encoding a recombinant polypeptide comprising isomaltase and one or both of a) a pre-protein signal peptide comprising an amino acid sequence of Formula III, Formula IV, Formula V or SEQ ID NO. 8, 9, 10, 11, 12, 13, 14, 15, or 16 and b) a pro-protein signal peptide comprising an amino acid sequence of Formula VI, Formula VII, Formula VIII, or SEQ ID NO. 18, 19, 20, 21, 22, 23, 24, or 25 and 2) a nucleic acid encoding a recombinant polypeptide comprising invertase and one or both of a) a pre-protein signal peptide comprising an amino acid sequence of Formula III, Formula IV, Formula V, or SEQ ID NO. 8, 9, 10, 11, 12, 13, 14, 15, or 16 and b) a pro-protein signal peptide comprising an amino acid sequence of Formula VI, Formula VII, Formula VIII, or SEQ ID NO. 18, 19, 20, 21, 22, 23, 24, or 25, thereby treating the deficiency.

In some embodiments, a method of treating a sucrase/invertase and/or isomaltase deficiency is provided, the method comprising administering to a subject in need thereof a Trichoderma yeast (e.g., T. reesei or T. viride), genetically modified with one or both of 1) a nucleic acid encoding a recombinant polypeptide comprising isomaltase and one or both of a) a pre-protein signal peptide comprising an amino acid sequence of Formula IX or SEQ ID NO. 31, 32, or 33 and b) a pro-protein signal peptide comprising an amino acid sequence of Formula X, Formula XI, or SEQ ID NO. 34, 35, 36, 37, or 38 and 2) a nucleic acid encoding a recombinant polypeptide comprising invertase and one or both of a) a pre-protein signal peptide comprising an amino acid sequence of Formula IX or SEQ ID NO. 31, 32, or 33 and b) a pro-protein signal peptide comprising an amino acid sequence of Formula X, Formula XI, or SEQ ID NO. 34, 35, 36, 37, or 38, thereby treating the deficiency.

In some embodiments, a method of treating a sucrase/invertase and/or isomaltase deficiency is provided, the method comprising administering to a subject in need thereof an Aspergillus yeast (e.g., A. niger), genetically modified with one or both of 1) a nucleic acid encoding a recombinant polypeptide comprising isomaltase and one or both of a) a pre-protein signal peptide comprising an amino acid sequence of Formula XIII or SEQ ID NO. 70, 71, 72, or 73 and b) a pro-protein signal peptide comprising an amino acid sequence of Formula XIV, Formula XV, or SEQ ID NO. 74 or 75 and 2) a nucleic acid encoding a recombinant polypeptide comprising invertase and one or both of a) a pre-protein signal peptide comprising an amino acid sequence of Formula XIII or SEQ ID NO. 70, 71, 72, or 73 and b) a pro-protein signal peptide comprising an amino acid sequence of Formula XIV, Formula XV, or SEQ ID NO. 74 or 75, thereby treating the deficiency.

In some embodiments, the sucrase/invertase and/or isomaltase deficiency may be, for example, congenital sucrase-isomaltase deficiency. In any embodiment where a subject has both a sucrase/invertase and isomaltase deficiency and it is desired to administer engineered yeast to express both enzymes, the same yeast strain may be used to express both enzymes or one yeast strain may be used to express invertase and another yeast strain may be used to express isomaltase. In some embodiments, administration of both enzymes is performed utilizing one yeast strain to express both enzymes. In some embodiments, administration of both enzymes is performed utilizing one yeast strain to express invertase and another yeast strain to express isomaltase.

In some embodiments, administering may be performed via any route. In some embodiments, the route of administration is oral or topical. The therapeutically effective amount of engineered yeast may be, for example, about 100 CFUs to 10²⁰ CFUs, about 10³ to 10¹⁵ CFUs, 10⁴ to 10¹⁰ CFUs, or about 10² to about 10⁸ CFUs. In some embodiments, the therapeutically effective amount of engineered yeast is from about 100 CFUs to about 10²⁰ CFUs. In some embodiments, the therapeutically effective amount of engineered yeast is from about 10³ to about 10¹⁵ CFUs. In some embodiments, the therapeutically effective amount of engineered yeast is from about 100 CFUs, about 10³ CFUs, or about 10⁴ CFUs to about 10⁸ CFUs, about 10¹⁰ CFUs, about 10¹⁵ CFUs, or about 10²⁰ CFUs. In some embodiments, the therapeutically effective amount of engineered yeast is any amount of CFU that falls within any of the above ranges.

### Method of Treating Lactose Intolerance

In some embodiments, a method of treating a lactase deficiency or lactose-intolerance in a subject in need thereof is provided, the method comprising administering to the subject a therapeutically effective amount of an engineered yeast genetically modified to express a recombinant polypeptide comprising lactase (or a pro-drug or active variant thereof) and a synthetic signal peptide, thereby treating lactase deficiency or lactose-intolerance. In some embodiments, the synthetic signal peptide comprises one or both of a) an pre-protein amino acid sequence of Formula II, Formula III, Formula IV, Formula V, Formula IX, Formula XIII or SEQ ID NO. 1, 2, 3, 4, 5, 6, 7, 8, 9, 10, 11, 12, 13, 14, 15, 16, 28, 31, 32, 33, 55, 70, 71, 72, or 73; and b) a pro-protein amino acid sequence of Formula VI, Formula VII, Formula VIII, Formula X, Formula XI, Formula XIV, Formula XV or SEQ ID NO. 17, 18, 19, 20, 21, 22, 23, 24, 25, 27, 29, 34, 35, 36, 37, 38, 56, 57, 58, 74, or 75. In some embodiments, the engineered yeast may be any strain as disclosed herein. In some embodiments, the engineered yeast is selected from the group comprising Kluyveromyces (e.g., K. lactis), Pichia (e.g., P. pastoris), Saccharomyces (e.g., S. cerevisiae, S. boulardii), Trichoderma (e.g., T. reesei, T. viride), and Aspergillus (e.g., A. niger).

In some embodiments, a method of treating a lactase deficiency/lactose-intolerance is provided, the method comprising administering to a subject in need thereof a Kluyveromyces yeast (e.g., K. lactis), genetically modified with a nucleic acid encoding a recombinant polypeptide comprising lactase and one or both of a) a pre-protein signal peptide comprising an amino acid sequence of Formula I or SEQ ID NO. 1 and b) a pro-protein signal peptide comprising an amino acid sequence of Formula VI or SEQ ID NO. 20 or 21, thereby treating the deficiency.

In some embodiments, a method of treating a lactase deficiency/lactose-intolerance is provided, the method comprising administering to a subject in need thereof Pichia yeast (e.g., P. pastoris), genetically modified with a nucleic acid encoding a recombinant polypeptide comprising lactase and one or both of a) a pre-protein signal peptide comprising an amino acid sequence of Formula II or SEQ ID NO. 2, 3, 4, 5, 6, or 7 and b) a pro-protein signal peptide comprising an amino acid sequence of Formula VI or SEQ ID NO. 17, 20 or 21, thereby treating the deficiency.

In some embodiments, a method of treating a lactase deficiency/lactose-intolerance is provided, the method comprising administering to a subject in need thereof a Saccharomyces yeast (e.g., S. cerevisiae or S. boulardii), genetically modified with a nucleic acid encoding a recombinant polypeptide comprising lactase and one or both of a) a pre-protein signal peptide comprising an amino acid sequence of Formula III, Formula IV, Formula V, or SEQ ID NO. 8, 9, 10, 11, 12, 13, 14, 15, or 16 and b) a pro-protein signal peptide comprising an amino acid sequence of Formula VI, Formula VII, Formula VIII, or SEQ ID NO. 18, 19, 20, 21, 22, 23, 24, or 25, thereby treating the deficiency.

In some embodiments, a method of treating a lactase deficiency/lactose-intolerance is provided, the method comprising administering to a subject in need thereof a Trichoderma yeast (e.g., T. reesei or T. viride), genetically modified with a nucleic acid encoding a recombinant polypeptide comprising lactase and one or both of a) a pre-protein signal peptide comprising an amino acid sequence of Formula IX or SEQ ID NO. 31, 32, or 33 and b) a pro-protein signal peptide comprising an amino acid sequence of Formula X, Formula XI, or SEQ ID NO. 34, 35, 36, 37, or 38, thereby treating the deficiency.

In some embodiments, a method of treating a lactase deficiency/lactose-intolerance is provided, the method comprising administering to a subject in need thereof an Aspergillus yeast (e.g., A. niger), genetically modified with a nucleic acid encoding a recombinant polypeptide comprising lactase and one or both of a) a pre-protein signal peptide comprising an amino acid sequence of Formula XIII or SEQ ID NO. 70, 71, 72, or 73 and b) a pro-protein signal peptide comprising an amino acid sequence of Formula XIV, Formula XV, or SEQ ID NO. 74 or 75, thereby treating the deficiency.

In some embodiments, administering may be performed via any route. In some embodiments, the route of administration is oral or topical. The therapeutically effective amount of engineered yeast may be, for example, about 100 CFUs to 10²⁰ CFUs, about 10³ to 10¹⁵ CFUs, 10⁴ to 10¹⁰ CFUs, or about 10² to about 10⁸ CFUs. In some embodiments, the therapeutically effective amount of engineered yeast is from about 100 CFUs to about 10²⁰ CFUs. In some embodiments, the therapeutically effective amount of engineered yeast is from about 10³ to about 10¹⁵ CFUs. In some embodiments, the therapeutically effective amount of engineered yeast is from about 100 CFUs, about 10³ CFUs, or about 10⁴ CFUs to about 10⁸ CFUs, about 10¹⁰ CFUs, about 10¹⁵ CFUs, or about 10²⁰ CFUs. In some embodiments, the therapeutically effective amount of engineered yeast is any amount of CFU that falls within any of the above ranges.

### Method of Treating Pancreatic Disorders

In some embodiments, a method of treating a pancreatic disorder, such as pancreatitis or exocrine pancreatic insufficiency, in a subject in need thereof is provided, the method comprising administering to the subject a therapeutically effective amount of an engineered yeast genetically modified to express a recombinant polypeptide comprising one or more of triacylglycerol lipase, colipase, alpha-amylase, trypsin, and chymotrypsin and a synthetic signal peptide, thereby treating the disorder. In some embodiments, the synthetic signal peptide comprises one or both of a) an pre-protein amino acid sequence of Formula II, Formula III, Formula IV, Formula V, Formula IX, Formula XIII or SEQ ID NO. 1, 2, 3, 4, 5, 6, 7, 8, 9, 10, 11, 12, 13, 14, 15, 16, 28, 31, 32, 33, 55, 70, 71, 72, or 73; and b) a pro-protein amino acid sequence of Formula VI, Formula VII, Formula VIII, Formula X, Formula XI, Formula XIV, Formula XV or SEQ ID NO. 17, 18, 19, 20, 21, 22, 23, 24, 25, 27, 29, 34, 35, 36, 37, 38, 56, 57, 58, 74, or 75. In some embodiments, the engineered yeast may be any strain as disclosed herein. In some embodiments, the engineered yeast is selected from the group comprising Kluyveromyces (e.g., K. lactis), Pichia (e.g., P. pastoris), Saccharomyces (e.g., S. cerevisiae, S. boulardii), Trichoderma (e.g., T. reesei, T. viride), and Aspergillus (e.g., A. niger). In some embodiments, the engineered yeast is genetically modified to express a recombinant polypeptide comprising triacylglycerol lipase and a synthetic signal peptide as provided for herein, and is effective for treating one or both of pancreatitis or exocrine pancreatic insufficiency. In some embodiments, the engineered yeast is genetically modified to express a recombinant polypeptide comprising colipase and a synthetic signal peptide as provided for herein, and is effective for treating one or both of pancreatitis or exocrine pancreatic insufficiency. In some embodiments, the engineered yeast is genetically modified to express a recombinant polypeptide comprising alpha-amylase and a synthetic signal peptide as provided for herein, and is effective for treating one or both of pancreatitis or exocrine pancreatic insufficiency. In some embodiments, the engineered yeast is genetically modified to express a recombinant polypeptide comprising trypsin and a synthetic signal peptide as provided for herein, and is effective for treating one or both of pancreatitis or exocrine pancreatic insufficiency. In some embodiments, the engineered yeast is genetically modified to express a recombinant polypeptide comprising chymotrypsin and a synthetic signal peptide as provided for herein, and is effective for treating one or both of pancreatitis or exocrine pancreatic insufficiency.

In some embodiments, a method of treating pancreatitis or exocrine pancreatic insufficiency is provided, the method comprising administering to a subject in need thereof a Kluyveromyces yeast (e.g., K. lactis), genetically modified with a nucleic acid encoding a recombinant polypeptide comprising 1) one or more of triacylglycerol lipase, colipase, alpha-amylase, trypsin, and chymotrypsin and 2) one or both of a) a pre-protein signal peptide comprising an amino acid sequence of Formula I or SEQ ID NO. 1 and b) a pro-protein signal peptide comprising an amino acid sequence of Formula VII or SEQ ID NO. 20 or 21, thereby treating the disorder.

In some embodiments, a method of treating pancreatitis or exocrine pancreatic insufficiency is provided, the method comprising administering to a subject in need thereof Pichia yeast (e.g., P. pastoris), genetically modified with a nucleic acid encoding a recombinant polypeptide comprising 1) one or more of triacylglycerol lipase, colipase, alpha-amylase, trypsin, and chymotrypsin and 2) one or both of a) a pre-protein signal peptide comprising an amino acid sequence of Formula II or SEQ ID NO. 2, 3, 4, 5, 6, or 7 and b) a pro-protein signal peptide comprising an amino acid sequence of Formula VI or SEQ ID NO. 17, 20 or 21, thereby treating the disorder.

In some embodiments, a method of treating pancreatitis or exocrine pancreatic insufficiency is provided, the method comprising administering to a subject in need thereof a Saccharomyces yeast (e.g., S. cerevisiae or S. boulardii), genetically modified with a nucleic acid encoding a recombinant polypeptide comprising 1) one or more of triacylglycerol lipase, colipase, alpha-amylase, trypsin, and chymotrypsin and 2) one or both of a) a pre-protein signal peptide comprising an amino acid sequence of Formula III, Formula IV, Formula V or SEQ ID NO. 8, 9, 10, 11, 12, 13, 14, 15, or 16 and b) a pro-protein signal peptide comprising an amino acid sequence of Formula VI, Formula VII, Formula VIII, or SEQ ID NO. 18, 19, 20, 21, 22, 23, 24, or 25, thereby treating the disorder.

In some embodiments, a method of treating pancreatitis or exocrine pancreatic insufficiency is provided, the method comprising administering to a subject in need thereof a Trichoderma yeast (e.g., T. reesei or T. viride), genetically modified with a nucleic acid encoding a recombinant polypeptide comprising 1) one or more of triacylglycerol lipase, colipase, alpha-amylase, trypsin, and chymotrypsin and 2) one or both of a) a pre-protein signal peptide comprising an amino acid sequence of Formula IX or SEQ ID NO. 31, 32, or 33 and b) a pro-protein signal peptide comprising an amino acid sequence of Formula X, Formula XI, or SEQ ID NO. 34, 35, 36, 37, or 38, thereby treating the disorder.

In some embodiments, a method of treating pancreatitis or exocrine pancreatic insufficiency is provided, the method comprising administering to a subject in need thereof an Aspergillus yeast (e.g., A. niger), genetically modified with a nucleic acid encoding a recombinant polypeptide comprising 1) one or more of triacylglycerol lipase, colipase, alpha-amylase, trypsin, and chymotrypsin and 2) one or both of a) a pre-protein signal peptide comprising an amino acid sequence of Formula XIII or SEQ ID NO. 70, 71, 72, or 73 and b) a pro-protein signal peptide comprising an amino acid sequence of Formula XIV, Formula XV, or SEQ ID NO. 74 or 75, thereby treating the disorder.

In some embodiments, administering may be performed via any route. In some embodiments, the route of administration is oral or topical. The therapeutically effective amount of engineered yeast may be, for example, about 100 CFUs to 10²⁰ CFUs, about 10³ to 10¹⁵ CFUs, 10⁴ to 10¹⁰ CFUs, or about 10² to about 10⁸ CFUs. In some embodiments, the therapeutically effective amount of engineered yeast is from about 100 CFUs to about 10²⁰ CFUs. In some embodiments, the therapeutically effective amount of engineered yeast is from about 10³ to about 10¹⁵ CFUs. In some embodiments, the therapeutically effective amount of engineered yeast is from about 100 CFUs, about 10³ CFUs, or about 10⁴ CFUs to about 10⁸ CFUs, about 10¹⁰ CFUs, about 10¹⁵ CFUs, or about 10²⁰ CFUs. In some embodiments, the therapeutically effective amount of engineered yeast is any amount of CFU that falls within any of the above ranges.

### Method of Treating Celiac Disease/Gluten Intolerance/Refractory Sprue

In some embodiments, a method of treating a deficiency of one or more of Aspergillus niger prolyl endoprotease (An-PEP), Myxococcus xanthus prolyl endopeptidase (Mx-PEP), Aspergillus tubigensis prolyl endopeptidase, subtilisin, sedolisin, and larozotide in a subject in need thereof is provided, the method comprising administering to the subject a therapeutically effective amount of an engineered yeast genetically modified to express a recombinant polypeptide comprising one or more of An-PEP, Mx-PEP, Aspergillus tubigensis prolyl endopeptidase, subtilisin, sedolisin, and larozotide (or a pro-drug or active variant thereof) and a synthetic signal peptide, thereby treating the deficiency. In some embodiments, the synthetic signal peptide comprises one or both of a) a pre-protein amino acid sequence of Formula II, Formula III, Formula IV, Formula V, Formula IX, Formula XIII or SEQ ID NO. 1, 2, 3, 4, 5, 6, 7, 8, 9, 10, 11, 12, 13, 14, 15, 16, 28, 31, 32, 33, 55, 70, 71, 72, or 73; and b) a pro-protein amino acid sequence of Formula VI, Formula VII, Formula VIII, Formula X, Formula XI, Formula XIV, Formula XV or SEQ ID NO. 17, 18, 19, 20, 21, 22, 23, 24, 25, 27, 29, 34, 35, 36, 37, 38, 56, 57, 58, 74, or 75. In some embodiments, the engineered yeast may be any strain as disclosed herein. In some embodiments, the yeast strain is selected from the group comprising Kluyveromyces (e.g., K. lactis), Pichia (e.g., P. pastoris), Saccharomyces (e.g., S. cerevisiae, S. boulardii), Trichoderma (e.g., T. reesei, T. viride), and Aspergillus (e.g., A. niger). In some embodiments, the recombinant polypeptide comprises An-PEP and a synthetic signal peptide as provided for herein, and the engineered yeast is effective to treat Celiac Disease, Gluten Intolerance, or refractory sprue. In some embodiments, the recombinant polypeptide comprises Mx-PEP and a synthetic signal peptide as provided for herein, and the engineered yeast is effective to treat Celiac Disease, Gluten Intolerance, or refractory sprue. In some embodiments, the recombinant polypeptide comprises Aspergillus tubigensis prolyl endopeptidase and a synthetic signal peptide as provided for herein, and the engineered yeast is effective to treat Celiac Disease, Gluten Intolerance, or refractory sprue. In some embodiments, the recombinant polypeptide comprises subtilisin and a synthetic signal peptide as provided for herein, and the engineered yeast is effective to treat Celiac Disease, Gluten Intolerance, or refractory sprue. In some embodiments, the recombinant polypeptide comprises sedolisin and a synthetic signal peptide as provided for herein, and the engineered yeast is effective to treat Celiac Disease, Gluten Intolerance, or refractory sprue. In some embodiments, the recombinant polypeptide comprises larozotide and a synthetic signal peptide as provided for herein, and the engineered yeast is effective to treat Celiac Disease, Gluten Intolerance, or refractory sprue.

In some embodiments, a method of treating one or more of Celiac Disease, gluten intolerance, and refractory sprue is provided, the method comprising administering to a subject in need thereof Kluyveromyces yeast (e.g., K. lactis), genetically modified with a nucleic acid encoding a recombinant polypeptide comprising 1) one or more of An-PEP, Mx-PEP, Aspergillus tubigensis prolyl endopeptidase, subtilisin, sedolisin, and larozotide and 2) one or both of a) a pre-protein signal peptide comprising an amino acid sequence of Formula I or SEQ ID NO. 1 and b) a pro-protein signal peptide comprising an amino acid sequence of Formula VI or SEQ ID NO. 20, 21, thereby treating the disease or disorder.

In some embodiments, a method of treating one or more of Celiac Disease, gluten intolerance, and refractory sprue is provided, the method comprising administering to a subject in need thereof Pichia yeast (e.g., P. pastoris), genetically modified with a nucleic acid encoding a recombinant polypeptide comprising 1) one or more of An-PEP, Mx-PEP, Aspergillus tubigensis prolyl endopeptidase, subtilisin, sedolisin, and larozotide and 2) one or both of a) a pre-protein signal peptide comprising an amino acid sequence of Formula II or SEQ ID NO. 2, 3, 4, 5, 6, or 7 and b) a pro-protein signal peptide comprising an amino acid sequence of Formula VI or SEQ ID NO. 17, 20 or 21 or Var. Seq. 6, thereby treating the disease or disorder.

In some embodiments, a method of treating one or more of Celiac Disease, gluten intolerance, and refractory sprue is provided, the method comprising administering to a subject in need thereof a Saccharomyces yeast (e.g., S. cerevisiae or S. boulardii), genetically modified with a nucleic acid encoding a recombinant polypeptide comprising 1) one or more of An-PEP, Mx-PEP, Aspergillus tubigensis prolyl endopeptidase, subtilisin, sedolisin, and larozotide and 2) one or both of a) a pre-protein signal peptide comprising an amino acid sequence of Formula III, Formula IV, Formula V, or SEQ ID NO. 8, 9, 10, 11, 12, 13, 14, 15, or 16 and b) a pro-protein signal peptide comprising an amino acid sequence of Formula VI, Formula VII, Formula VIII, or SEQ ID NO. 18, 19, 20, 21, 22, 23, 24, 25, thereby treating the disease or disorder.

In some embodiments, a method of treating one or more of Celiac Disease, gluten intolerance, and refractory sprue is provided, the method comprising administering to a subject in need thereof a Trichoderma yeast (e.g., T. reesei or T. viride), genetically modified with a nucleic acid encoding a recombinant polypeptide comprising 1) one or more of An-PEP, Mx-PEP, Aspergillus tubigensis prolyl endopeptidase, subtilisin, sedolisin, and larozotide and 2) one or both of a) a pre-protein signal peptide comprising an amino acid sequence of Formula IX or SEQ ID NO. 31, 32, or 33 and b) a pro-protein signal peptide comprising an amino acid sequence of Formula X, Formula XI, or SEQ ID NO. 34, 35, 36, 37, or 38, thereby treating the disease or disorder.

In some embodiments, a method of treating one or more of Celiac Disease, gluten intolerance, and refractory sprue is provided, the method comprising administering to a subject in need thereof an Aspergillus yeast (e.g., A. niger), genetically modified with a nucleic acid encoding a recombinant polypeptide comprising 1) one or more of An-PEP, Mx-PEP, Aspergillus tubigensis prolyl endopeptidase, subtilisin, sedolisin, and larozotide and 2) one or both of a) a pre-protein signal peptide comprising an amino acid sequence of Formula XIII or SEQ ID NO. 70, 71, 72, or 73 and b) a pro-protein signal peptide comprising an amino acid sequence of Formula XIV, Formula XV, or SEQ ID NO. 74 or 75, thereby treating the disease or disorder.

In some embodiments, administering may be performed via any route. In some embodiments, the route of administration is oral or topical. The therapeutically effective amount of engineered yeast may be, for example, about 100 CFUs to 10²⁰ CFUs, about 10³ to 10¹⁵ CFUs, 10⁴ to 10¹⁰ CFUs, or about 10² to about 10⁸ CFUs. In some embodiments, the therapeutically effective amount of engineered yeast is from about 100 CFUs to about 10²⁰ CFUs. In some embodiments, the therapeutically effective amount of engineered yeast is from about 10³ to about 10¹⁵ CFUs. In some embodiments, the therapeutically effective amount of engineered yeast is from about 100 CFUs, about 10³ CFUs, or about 10⁴ CFUs to about 10⁸ CFUs, about 10¹⁰ CFUs, about 10¹⁵ CFUs, or about 10²⁰ CFUs. In some embodiments, the therapeutically effective amount of engineered yeast is any amount of CFU that falls within any of the above ranges.

### Methods of Treating Enteropeptidase/Enterokinase Deficiency

Enterokinase or enteropeptidase deficiency is an autosomal recessive disorder characterized by severe protein malabsorption in early infancy and may be treated by an engineered yeast according to the present disclosure. Accordingly, in some embodiments, a method of treating enterokinase/enteropeptidase deficiency in a subject in need thereof is provided, the method comprising administering to the subject a therapeutically effective amount of an engineered yeast genetically modified to express a recombinant polypeptide comprising one or both of enteropeptidase (enterokinase) and proenteropeptidase and a synthetic signal peptide, thereby treating the disorder. In some embodiments, the synthetic signal peptide comprises one or both of a) an pre-protein amino acid sequence of Formula II, Formula III, Formula IV, Formula V, Formula IX, Formula XIII or SEQ ID NO. 1, 2, 3, 4, 5, 6, 7, 8, 9, 10, 11, 12, 13, 14, 15, 16, 28, 31, 32, 33, 55, 70, 71, 72, or 73; and b) a pro-protein amino acid sequence of Formula VI, Formula VII, Formula VIII, Formula X, Formula XI, Formula XIV, Formula XV or SEQ ID NO. 17, 18, 19, 20, 21, 22, 23, 24, 25, 27, 29, 34, 35, 36, 37, 38, 56, 57, 58, 74, or 75. In some embodiments, the engineered yeast may be any strain as disclosed herein. In some embodiments, the engineered yeast is selected from the group comprising Kluyveromyces (e.g., K. lactis), Pichia (e.g., P. pastoris), Saccharomyces (e.g., S. cerevisiae, S. boulardii), Trichoderma (e.g., T. reesei, T. viride), and Aspergillus (e.g., A. niger).

In some embodiments, a method of treating enterokinase or enteropeptidase deficiency is provided, the method comprising administering to a subject in need thereof Kluyveromyces yeast (e.g., K. lactis), genetically modified with a nucleic acid encoding a recombinant polypeptide comprising 1) one or both of enteropeptidase/enterokinase and proenteropeptidase and 2) one or both of a) a pre-protein signal peptide comprising an amino acid sequence of Formula I or SEQ ID NO. 1 and b) a pro-protein signal peptide comprising an amino acid sequence of Formula VI or SEQ ID NO. 17, 20, or 21, thereby treating the disorder.

In some embodiments, a method of treating enterokinase or enteropeptidase deficiency is provided, the method comprising administering to a subject in need thereof Pichia yeast (e.g., P. pastoris), genetically modified with a nucleic acid encoding a recombinant polypeptide comprising 1) one or both of enteropeptidase/enterokinase and proenteropeptidase and 2) one or both of a) a pre-protein signal peptide comprising an amino acid sequence of Formula II or SEQ ID NO. 2, 3, 4, 5, 6, or 7 and b) a pro-protein signal peptide comprising an amino acid sequence of Formula VI or SEQ ID NO. 17, 20 or 21, thereby treating the disorder.

In some embodiments, a method of treating enterokinase or enteropeptidase deficiency is provided, the method comprising administering to a subject in need thereof a Saccharomyces yeast (e.g., S. cerevisiae or S. boulardii), genetically modified with a nucleic acid encoding a recombinant polypeptide comprising 1) one or both of enteropeptidase/enterokinase and proenteropeptidase and 2) one or both of a) a pre-protein signal peptide comprising an amino acid sequence of Formula III, Formula IV, Formula V, or SEQ ID NO. 8, 9, 10, 11, 12, 13, 14, 15, or 16 and b) a pro-protein signal peptide comprising an amino acid sequence of Formula VI, Formula VII, Formula VIII, or SEQ ID NO. 18, 19, 20, 21, 22, 23, 24, or 25, or, thereby treating the disorder.

In some embodiments, a method of treating enterokinase or enteropeptidase deficiency is provided, the method comprising administering to a subject in need thereof a Trichoderma yeast (e.g., T. reesei or T. viride), genetically modified with a nucleic acid encoding a recombinant polypeptide comprising 1) one or both of enteropeptidase/enterokinase and proenteropeptidase and 2) one or both of a) a pre-protein signal peptide comprising an amino acid sequence of Formula IX or SEQ ID NO. 31, 32, or 33 and b) a pro-protein signal peptide comprising an amino acid sequence of Formula X, Formula XI, or SEQ ID NO. 34, 35, 36, 37, or 38, thereby treating the disorder.

In some embodiments, a method of treating enterokinase or enteropeptidase deficiency is provided, the method comprising administering to a subject in need thereof an Aspergillus yeast (e.g., A. niger), genetically modified with a nucleic acid encoding a recombinant polypeptide comprising 1) one or both of enteropeptidase/enterokinase and proenteropeptidase and 2) one or both of a) a pre-protein signal peptide comprising an amino acid sequence of Formula XIII or SEQ ID NO. 70, 71, 72, or 73 and b) a pro-protein signal peptide comprising an amino acid sequence of Formula XIV, Formula XV, or SEQ ID NO. 74 or 75, thereby treating the disorder.

In some embodiments, administering may be performed via any route. In some embodiments, the route of administration is oral or topical. The therapeutically effective amount of engineered yeast may be, for example, about 100 CFUs to 10²⁰ CFUs, about 10³ to 10¹⁵ CFUs, 10⁴ to 10¹⁰ CFUs, or about 10² to about 10⁸ CFUs. In some embodiments, the therapeutically effective amount of engineered yeast is from about 100 CFUs to about 10²⁰ CFUs. In some embodiments, the therapeutically effective amount of engineered yeast is from about 10³ to about 10¹⁵ CFUs. In some embodiments, the therapeutically effective amount of engineered yeast is from about 100 CFUs, about 10³ CFUs, or about 10⁴ CFUs to about 10⁸ CFUs, about 10¹⁰ CFUs, about 10¹⁵ CFUs, or about 10²⁰ CFUs. In some embodiments, the therapeutically effective amount of engineered yeast is any amount of CFU that falls within any of the above ranges.

### Methods of Treating Small Intestine Bacterial Overgrowth or a Bacterial Infection

In some embodiments, a method of treating bacterial infection or bacterial overgrowth in a subject in need thereof is provided, the method comprising administering to the subject a therapeutically effective amount of an engineered yeast genetically modified to express a recombinant polypeptide comprising 1) one or both of lysozyme and intestinal alkaline phosphatase and 2) a synthetic signal peptide, thereby treating the infection or overgrowth. In some embodiments, the synthetic signal peptide comprises one or both of a) an pre-protein amino acid sequence of Formula II, Formula III, Formula IV, Formula V, Formula IX, Formula XIII or SEQ ID NO. 2, 3, 4, 5, 6, 7, 8, 9, 10, 11, 12, 13, 14, 15, 16, 28, 31, 32, 33, 55, 70, 71, 72, or 73; and b) a pro-protein amino acid sequence of Formula VI, Formula VII, Formula VIII, Formula X, Formula XI, Formula XIV, Formula XV or SEQ ID NO. 17, 18, 19, 20, 21, 22, 23, 24, 25, 27, 29, 34, 35, 36, 37, 38, 56, 57, 58, 74, or 75. In some embodiments, the engineered yeast may be any strain as disclosed herein. In some embodiments, the engineered yeast is selected from the group comprising Kluyveromyces (e.g., K. lactis), Pichia (e.g., P. pastoris), Saccharomyces (e.g., S. cerevisiae, S. boulardii), Trichoderma (e.g., T. reesei, T. viride), and Aspergillus (e.g., A. niger). In some embodiments, the bacterial infection or overgrowth may include, but not be limited to, a small intestine bacterial overgrowth, which may be associated with diabetes, a C. difficile infection, and intestinal bacterial overgrowth associated with cystic fibrosis. In some embodiments, the bacterial infection may be caused by be any gram-positive or gram-negative bacteria, such as, but not limited to, an infection of Escherichia Coli (E. Coli), Clostridioides difficile, P. aeruginosa, Shigella, Salmonella, Vibrio cholera, or cryptosporidium.

In some embodiments, a method of treating a bacterial overgrowth or infection is provided, the method comprising administering to a subject in need thereof Kluyveromyces yeast (e.g., K. lactis), genetically modified with a nucleic acid encoding a recombinant polypeptide comprising 1) one or both of lysozyme and intestinal alkaline phosphatase and 2) and one or both of a) a pre-protein signal peptide comprising an amino acid sequence of Formula I or SEQ ID NO. 1 and b) a pro-protein signal peptide comprising an amino acid sequence of Formula VI or SEQ ID NO. 17, 20, or 21, thereby treating the infection or overgrowth.

In some embodiments, a method of treating a bacterial overgrowth or infection is provided, the method comprising administering to a subject in need thereof Pichia yeast (e.g., P. pastoris), genetically modified with a nucleic acid encoding a recombinant polypeptide comprising 1) one or both of lysozyme and intestinal alkaline phosphatase and 2) and one or both of a) a pre-protein signal peptide comprising an amino acid sequence of Formula II or SEQ ID NO. 2, 3, 4, 5, 6, or 7 and b) a pro-protein signal peptide comprising an amino acid sequence of Formula VI or SEQ ID NO. 17, 20 or 21, thereby treating the infection or overgrowth.

In some embodiments, a method of treating a bacterial overgrowth or infection is provided, the method comprising administering to a subject in need thereof a Saccharomyces yeast (e.g., S. cerevisiae or S. boulardii), genetically modified with a nucleic acid encoding a recombinant polypeptide comprising 1) one or both of lysozyme and intestinal alkaline phosphatase and 2) and one or both of a) a pre-protein signal peptide comprising an amino acid sequence of Formula III, Formula IV, Formula V, or SEQ ID NO. 8, 9, 10, 11, 12, 13, 14, 15 and b) a pro-protein signal peptide comprising an amino acid sequence of Formula VI, Formula VII, Formula VIII, or SEQ ID NO. 18, 19, 20, 21, 22, 23, 24, or 25, thereby treating the infection or overgrowth.

In some embodiments, a method of treating a bacterial overgrowth or infection is provided, the method comprising administering to a subject in need thereof a Trichoderma yeast (e.g., T. reesei or T. viride), genetically modified with a nucleic acid encoding a recombinant polypeptide comprising 1) one or both of lysozyme and intestinal alkaline phosphatase and 2) and one or both of a) a pre-protein signal peptide comprising an amino acid sequence of Formula IX or SEQ ID NO. 31, 32, or 33 and b) a pro-protein signal peptide comprising an amino acid sequence of Formula X, Formula XI, or SEQ ID NO. 34, 35, 36, 37, or 38, thereby treating the infection or overgrowth.

In some embodiments, a method of treating a bacterial overgrowth or infection is provided, the method comprising administering to a subject in need thereof an Aspergillus yeast (e.g., A. niger), genetically modified with a nucleic acid encoding a recombinant polypeptide comprising 1) one or both of lysozyme and intestinal alkaline phosphatase and 2) and one or both of a) a pre-protein signal peptide comprising an amino acid sequence of Formula XIII or SEQ ID NO. 70, 71, 72, or 73 and b) a pro-protein signal peptide comprising an amino acid sequence of Formula XIV, Formula XV, or SEQ ID NO. 74 or 75, thereby treating the infection or overgrowth.

In some embodiments, other antibacterial proteins that may be produced by an engineered yeast and therefore provide treatment for bacterial overgrowth or infection in a subject include human beta defensins, peptide antimicrobials of animal origin (e.g., magainin, dermaseptin, cateslytin), and peptide antimicrobials of microbe origin (e.g., nisin, sakacin). In some embodiments, administering may be performed via any route. In some embodiments, the route of administration is oral or topical. The therapeutically effective amount of engineered yeast may be, for example, about 100 CFUs to 10²⁰ CFUs, about 10³ to 10¹⁵ CFUs, 10⁴ to 10¹⁰ CFUs, or about 10² to about 10⁸ CFUs. In some embodiments, the therapeutically effective amount of engineered yeast is from about 100 CFUs to about 10²⁰ CFUs. In some embodiments, the therapeutically effective amount of engineered yeast is from about 10³ to about 10¹⁵ CFUs. In some embodiments, the therapeutically effective amount of engineered yeast is from about 100 CFUs, about 10³ CFUs, or about 10⁴ CFUs to about 10⁸ CFUs, about 10¹⁰ CFUs, about 10¹⁵ CFUs, or about 10²⁰ CFUs. In some embodiments, the therapeutically effective amount of engineered yeast is any amount of CFU that falls within any of the above ranges.

In some embodiments, the method of treating a bacterial infection with an engineered yeast genetically modified to express lysozyme, as described herein, may further comprise administering an antibacterial agent in combination with the engineered yeast. For example, a bacterial infection may be treated by administering a therapeutically effective amount of an engineered yeast genetically modified to express a recombinant polypeptide comprising a synthetic signal peptide and lysozyme and a therapeutically effective amount of an antibacterial agent. In some embodiments, the antibacterial agent is selected from the group comprising quinupristin, piperacillin, penicillin, clarithromycin, nitrofurantoin, ciprofloxacin, telithromycin, metronidazole, levofloxacin, erythromycin, theophylline, gemifloxacin, tetracycline, azithromycin, delafloxacin, eravacycline, moxifloxacin, dalbavancin, amoxicillin, fidaxomicin, tigecycline, ceftriaxone, minocycline, rifapentine, clindamycin, ceftazidime, oritayancin, norfloxacin, doxycycline, cefuroxime, tobramycin, ceftibuten, gentamicin, cefotaxime, vancomycin, telavancin, daptomycin, cephalexin, fofomycin, tedizolid, aztreonam, nafcillin, phenytoin, ertapenem, cefazolin, isoniazid, doripenem, rifabutin, meropenem, linezolid, oflaxacin, cefoxitin, oxacillin, warfarin, neomycin, rifampin, cefepime, and digoxin. In some embodiments, the antibacterial agent can be administered by any route, such as oral, topical, intranasal, mucosal, otic, parenteral, or the like.

### Methods of Treating Gastrointestinal Disorders

In some embodiments, a method of treating inflammatory gastrointestinal disorders in a subject in need thereof is provided, the method comprising administering to the subject a therapeutically effective amount of an engineered yeast genetically modified to express a recombinant polypeptide comprising intestinal alkaline phosphatase and a synthetic signal peptide. In some embodiments, the synthetic signal peptide comprises one or both of a) an pre-protein amino acid sequence of Formula II, Formula III, Formula IV, Formula V, Formula IX, Formula XIII or SEQ ID NO. 2, 3, 4, 5, 6, 7, 8, 9, 10, 11, 12, 13, 14, 15, 16, 28, 31, 32, 33, 55, 70, 71, 72, or 73; and b) a pro-protein amino acid sequence of Formula VI, Formula VII, Formula VIII, Formula X, Formula XI, Formula XIV, Formula XV or SEQ ID NO. 17, 18, 19, 20, 21, 22, 23, 24, 25, 27, 29, 34, 35, 36, 37, 38, 56, 57, 58, 74, or 75. In some embodiments, the engineered yeast may be any strain as disclosed herein. In some embodiments, the engineered yeast is selected from the group comprising Kluyveromyces (e.g., K. lactis), Pichia (e.g., P. pastoris), Saccharomyces (e.g., S. cerevisiae, S. boulardii), Trichoderma (e.g., T. reesei, T. viride), and Aspergillus (e.g., A. niger). In some embodiments, the inflammatory gastrointestinal disorder is selected from the group including, but not limited to, inflammatory bowel disease (IBD), irritable bowel syndrome (IBS), and necrotizing enterocolitis.

In some embodiments, a method for treating an inflammatory gastrointestinal disorder is provided, the method comprising administering to a subject in need thereof Kluyveromyces yeast (e.g., K. lactis), genetically modified with a nucleic acid encoding a recombinant polypeptide comprising intestinal alkaline phosphatase and one or both of a) a pre-protein signal peptide comprising an amino acid sequence of Formula I or SEQ ID NO. 1 and b) a pro-protein signal peptide comprising an amino acid sequence of Formula VI or SEQ ID NO. 17, 20, or 21, thereby treating the disorder. In some embodiments, the inflammatory gastrointestinal disorder is selected from the group comprising IBS, IBD, and necrotizing enterocolitis.

In some embodiments, a method for treating an inflammatory gastrointestinal disorder is provided, the method comprising administering to a subject in need thereof Pichia yeast (e.g., P. pastoris), genetically modified with a nucleic acid encoding a recombinant polypeptide comprising intestinal alkaline phosphatase and one or both of a) a pre-protein signal peptide comprising an amino acid sequence of Formula II or SEQ ID NO. 2, 3, 4, 5, 6, or 7 and b) a pro-protein signal peptide comprising an amino acid sequence of Formula VI or SEQ ID NO. 17, 20 or 21, thereby treating the disorder. In some embodiments, the inflammatory gastrointestinal disorder is selected from the group comprising IBS, IBD, and necrotizing enterocolitis.

In some embodiments, a method for treating an inflammatory gastrointestinal disorder is provided, the method comprising administering to a subject in need thereof a Saccharomyces yeast (e.g., S. cerevisiae or S. boulardii), genetically modified with a nucleic acid encoding a recombinant polypeptide comprising intestinal alkaline phosphatase and one or both of a) a pre-protein signal peptide comprising an amino acid sequence of Formula III, Formula IV, Formula V, or SEQ ID NO. 8, 9, 10, 11, 12, 13, 14, 15, or 16 and b) a pro-protein signal peptide comprising an amino acid sequence of Formula VI, Formula VII, Formula VIII, or SEQ ID NO. 18, 19, 20, 21, 22, 23, 24, or 25, thereby treating the disorder. In some embodiments, the inflammatory gastrointestinal disorder is selected from the group comprising IBS, IBD, and necrotizing enterocolitis.

In some embodiments, a method for treating an inflammatory gastrointestinal disorder is provided, the method comprising administering to a subject in need thereof a Trichoderma yeast (e.g., T. reesei or T. viride), genetically modified with a nucleic acid encoding a recombinant polypeptide comprising intestinal alkaline phosphatase and one or both of a) a pre-protein signal peptide comprising an amino acid sequence of Formula IX or SEQ ID NO. 31, 32, or 33 and b) a pro-protein signal peptide comprising an amino acid sequence of Formula X, Formula XI, or SEQ ID NO. 34, 35, 36, 37, or 38, thereby treating the disorder. In some embodiments, the inflammatory gastrointestinal disorder is selected from the group comprising IBS, IBD, and necrotizing enterocolitis.

In some embodiments, a method for treating an inflammatory gastrointestinal disorder is provided, the method comprising administering to a subject in need thereof an Aspergillus yeast (e.g., A. niger), genetically modified with a nucleic acid encoding a recombinant polypeptide comprising intestinal alkaline phosphatase and one or both of a) a pre-protein signal peptide comprising an amino acid sequence of Formula XIII or SEQ ID NO. 70, 71, 72, or 73 and b) a pro-protein signal peptide comprising an amino acid sequence of Formula XIV, Formula XV, or SEQ ID NO. 74 or 75, thereby treating the disorder. In some embodiments, the inflammatory gastrointestinal disorder is selected from the group comprising IBS, IBD, and necrotizing enterocolitis.

In some embodiments, administering may be performed via any route. In some embodiments, the route of administration is oral or topical. The therapeutically effective amount of engineered yeast may be, for example, about 100 CFUs to 10²⁰ CFUs, about 10³ to 10¹⁵ CFUs, 10⁴ to 10¹⁰ CFUs, or about 10² to about 10⁸ CFUs. In some embodiments, the therapeutically effective amount of engineered yeast is from about 100 CFUs to about 10²⁰ CFUs. In some embodiments, the therapeutically effective amount of engineered yeast is from about 10³ to about 10¹⁵ CFUs. In some embodiments, the therapeutically effective amount of engineered yeast is from about 100 CFUs, about 10³ CFUs, or about 10⁴ CFUs to about 10⁸ CFUs, about 10¹⁰ CFUs, about 10¹⁵ CFUs, or about 10²⁰ CFUs. In some embodiments, the therapeutically effective amount of engineered yeast is any amount of CFU that falls within any of the above ranges.

### Methods of Treating Insulin Deficiency/Diabetes

An engineered yeast may be used to treat an insulin deficiency or disorder, such as type 1 and type 2 diabetes mellitus. Accordingly, in some embodiments, a method of treating type 1 or type 2 diabetes mellitus in a subject in need thereof is provided, the method comprising administering to the subject a therapeutically effective amount of an engineered yeast genetically modified to express a recombinant polypeptide comprising insulin (or a peptide analog or pro-drug thereof) and a synthetic signal peptide. In some embodiments, the synthetic signal peptide comprises one or both of a) an pre-protein amino acid sequence of Formula II, Formula III, Formula IV, Formula V, Formula IX, Formula XIII or SEQ ID NO. 1, 2, 3, 4, 5, 6, 7, 8, 9, 10, 11, 12, 13, 14, 15, 16, 28, 31, 32, 33, 55, 70, 71, 72, or 73; and b) a pro-protein amino acid sequence of Formula VI, Formula VII, Formula VIII, Formula X, Formula XI, Formula XIV, Formula XV or SEQ ID NO. 17, 18, 19, 20, 21, 22, 23, 24, 25, 27, 29, 34, 35, 36, 37, 38, 56, 57, 58, 74, or 75. In some embodiments, the engineered yeast may be any strain as disclosed herein. In some embodiments, the engineered yeast is selected from the group comprising Kluyveromyces (e.g., K. lactis), Pichia (e.g., P. pastoris), Saccharomyces (e.g., S. cerevisiae, S. boulardii), Trichoderma (e.g., T. reesei, T. viride), and Aspergillus (e.g., A. niger).

In some embodiments, a method of treating an insulin deficiency/diabetes is provided, the method comprising administering to a subject in need thereof Kluyveromyces yeast (e.g., K. lactis), genetically modified with a nucleic acid encoding a recombinant polypeptide comprising insulin (or a peptide analog or pro-drug thereof) and one or both of a) a pre-protein signal peptide comprising an amino acid sequence of Formula I or SEQ ID NO. 1 and b) a pro-protein signal peptide comprising an amino acid sequence of Formula VI or SEQ ID NO. 20 or 21, thereby treating the deficiency or disease.

In some embodiments, a method of treating an insulin deficiency/diabetes is provided, the method comprising administering to a subject in need thereof Pichia yeast (e.g., P. pastoris), genetically modified with a nucleic acid encoding a recombinant polypeptide comprising insulin (or a peptide analog or pro-drug thereof) and one or both of a) a pre-protein signal peptide comprising an amino acid sequence of Formula II or SEQ ID NO. 2, 3, 4, 5, 6, or 7 and b) a pro-protein signal peptide comprising an amino acid sequence of Formula VI or SEQ ID NO. 17, 20, or 21, thereby treating the deficiency or disease.

In some embodiments, a method of treating an insulin deficiency/diabetes is provided, the method comprising administering to a subject in need thereof a Saccharomyces yeast (e.g., S. cerevisiae or S. boulardii), genetically modified with a nucleic acid encoding a recombinant polypeptide comprising insulin (or a peptide analog or pro-drug thereof) and one or both of a) a pre-protein signal peptide comprising an amino acid sequence of Formula III, Formula IV, Formula V, or SEQ ID NO. 8, 9, 10, 11, 12, 13, 14, 15, or 16 and b) a pro-protein signal peptide comprising an amino acid sequence of Formula VI, Formula VII, Formula VIII, or SEQ ID NO. 18, 19, 20, 21, 22, 23, 24, 25, thereby treating the deficiency or disease.

In some embodiments, a method of treating an insulin deficiency/diabetes is provided, the method comprising administering to a subject in need thereof a Trichoderma yeast (e.g., T. reesei or T. viride), genetically modified with a nucleic acid encoding a recombinant polypeptide comprising insulin (or a peptide analog or pro-drug thereof) and one or both of a) a pre-protein signal peptide comprising an amino acid sequence of Formula IX or SEQ ID NO. 31, 32, or 33 and b) a pro-protein signal peptide comprising an amino acid sequence of Formula X, Formula XI, or SEQ ID NO. 34, 35, 36, 37, or 38, thereby treating the deficiency or disease.

In some embodiments, a method of treating an insulin deficiency/diabetes is provided, the method comprising administering to a subject in need thereof an Aspergillus yeast (e.g., A. niger), genetically modified with a nucleic acid encoding a recombinant polypeptide comprising insulin (or a peptide analog or pro-drug thereof) and one or both of a) a pre-protein signal peptide comprising an amino acid sequence of Formula XIII or SEQ ID NO. 70, 71, 72, or 73 and b) a pro-protein signal peptide comprising an amino acid sequence of Formula XIV, Formula XV, or SEQ ID NO. 74 or 75, thereby treating the deficiency or disease.

In some embodiments, administering may be performed via any route. In some embodiments, the route of administration is oral or topical. The therapeutically effective amount of engineered yeast may be, for example, about 100 CFUs to 10²⁰ CFUs, about 10³ to 10¹⁵ CFUs, 10⁴ to 10¹⁰ CFUs, or about 10² to about 10⁸ CFUs. In some embodiments, the therapeutically effective amount of engineered yeast is from about 100 CFUs to about 10²⁰ CFUs. In some embodiments, the therapeutically effective amount of engineered yeast is from about 10³ to about 10¹⁵ CFUs. In some embodiments, the therapeutically effective amount of engineered yeast is from about 100 CFUs, about 10³ CFUs, or about 10⁴ CFUs to about 10⁸ CFUs, about 10¹⁰ CFUs, about 10¹⁵ CFUs, or about 10²⁰ CFUs. In some embodiments, the therapeutically effective amount of engineered yeast is any amount of CFU that falls within any of the above ranges.

In some embodiments, a method of treating type 1 or type 2 diabetes mellitus in a subject in need thereof is provided, the method comprising administering to the subject a therapeutically effective amount of an engineered yeast genetically modified to express a recombinant polypeptide comprising an incretin and a synthetic signal peptide, thereby treating the type 1 or type 2 diabetes mellitus. In some embodiments, the synthetic signal peptide comprises one or both of a) an pre-protein amino acid sequence of Formula II, Formula III, Formula IV, Formula V, Formula IX, Formula XIII or SEQ ID NO. 1, 2, 3, 4, 5, 6, 7, 8, 9, 10, 11, 12, 13, 14, 15, 16, 28, 31, 32, 33, 55, 70, 71, 72, or 73; and b) a pro-protein amino acid sequence of Formula VI, Formula VII, Formula VIII, Formula X, Formula XI, Formula XIV, Formula XV or SEQ ID NO. 17, 18, 19, 20, 21, 22, 23, 24, 25, 27, 29, 34, 35, 36, 37, 38, 56, 57, 58, 74, or 75. In some embodiments, the engineered yeast may be any strain as disclosed herein. In some embodiments, the engineered yeast is selected from the group comprising Kluyveromyces (e.g., K. lactis), Pichia (e.g., P. pastoris), Saccharomyces (e.g., S. cerevisiae, S. boulardii), Trichoderma (e.g., T. reesei, T. viride), and Aspergillus (e.g., A. niger). In some embodiments, the incretin is selected from the group including, but not limited to, GLP-1, GLP-2, leptin, apelin, ghrelin, PYY, nesfatin, diaglutide, exenatide, liraglutide, semaglutide, sitagliptin, saxagliptin, alogliptin, linagliptin, and GIP.

In some embodiments, a method of treating an insulin deficiency/diabetes is provided, the method comprising administering to a subject in need thereof Kluyveromyces yeast (e.g., K. lactis), genetically modified with a nucleic acid encoding a recombinant polypeptide comprising an incretin and one or both of a) a pre-protein signal peptide comprising an amino acid sequence of Formula I or SEQ ID NO. 1 and b) a pro-protein signal peptide comprising an amino acid sequence of Formula VI or SEQ ID NO. 20 or 21, thereby treating the deficiency or disease. In some embodiments, the incretin is selected from the group including, but not limited to, GLP-1, GLP-2, leptin, apelin, ghrelin, PYY, nesfatin, diaglutide, exenatide, liraglutide, semaglutide, sitagliptin, saxagliptin, alogliptin, linagliptin, and GIP.

In some embodiments, a method of treating an insulin deficiency/diabetes is provided, the method comprising administering to a subject in need thereof Pichia yeast (e.g., P. pastoris), genetically modified with a nucleic acid encoding a recombinant polypeptide comprising an incretin and one or both of a) a pre-protein signal peptide comprising an amino acid sequence of Formula II or SEQ ID NO. 2, 3, 4, 5, 6, or 7 and b) a pro-protein signal peptide comprising an amino acid sequence of Formula VI or SEQ ID NO. 17, 20, or 21, thereby treating the deficiency or disease. In some embodiments, the incretin is selected from the group including, but not limited to, GLP-1, GLP-2, leptin, apelin, ghrelin, PYY, nesfatin, diaglutide, exenatide, liraglutide, semaglutide, sitagliptin, saxagliptin, alogliptin, linagliptin, and GIP.

In some embodiments, a method of treating an insulin deficiency/diabetes is provided, the method comprising administering to a subject in need thereof a Saccharomyces yeast (e.g., S. cerevisiae or S. boulardii), genetically modified with a nucleic acid encoding a recombinant polypeptide comprising an incretin and one or both of a) a pre-protein signal peptide comprising an amino acid sequence of Formula III, Formula IV, Formula V, or SEQ ID NO. 8, 9, 10, 11, 12, 13, 14, 15, or 16 and b) a pro-protein signal peptide comprising an amino acid sequence of Formula VI, Formula VII, Formula VIII, or SEQ ID NO. 18, 19, 20, 21, 22, 23, 24, 25, thereby treating the deficiency or disease. In some embodiments, the incretin is selected from the group including, but not limited to, GLP-1, GLP-2, leptin, apelin, ghrelin, PYY, nesfatin, diaglutide, exenatide, liraglutide, semaglutide, sitagliptin, saxagliptin, alogliptin, linagliptin, and GIP).

In some embodiments, a method of treating an insulin deficiency/diabetes is provided, the method comprising administering to a subject in need thereof a Trichoderma yeast (e.g., T. reesei or T. viride), genetically modified with a nucleic acid encoding a recombinant polypeptide comprising an incretin and one or both of a) a pre-protein signal peptide comprising an amino acid sequence of Formula IX or SEQ ID NO. 31, 32, or 33 and b) a pro-protein signal peptide comprising an amino acid sequence of Formula X, Formula XI, or SEQ ID NO. 34, 35, 36, 37, or 38, thereby treating the deficiency or disease. In some embodiments, the incretin is selected from the group including, but not limited to, GLP-1, GLP-2, leptin, apelin, ghrelin, PYY, nesfatin, diaglutide, exenatide, liraglutide, semaglutide, sitagliptin, saxagliptin, alogliptin, linagliptin, and GIP.

In some embodiments, a method of treating an insulin deficiency/diabetes is provided, the method comprising administering to a subject in need thereof an Aspergillus yeast (e.g., A. niger), genetically modified with a nucleic acid encoding a recombinant polypeptide comprising an incretin and one or both of a) a pre-protein signal peptide comprising an amino acid sequence of Formula XIII or SEQ ID NO. 70, 71, 72, or 73 and b) a pro-protein signal peptide comprising an amino acid sequence of Formula XIV, Formula XV, or SEQ ID NO. 74 or 75, thereby treating the deficiency or disease. In some embodiments, the incretin is selected from the group including, but not limited to, GLP-1, GLP-2, leptin, apelin, ghrelin, PYY, nesfatin, diaglutide, exenatide, liraglutide, semaglutide, sitagliptin, saxagliptin, alogliptin, linagliptin, and GIP.

In some embodiments, administering may be performed via any route. In some embodiments, the route of administration is oral or topical. The therapeutically effective amount of engineered yeast may be, for example, about 100 CFUs to 10²⁰ CFUs, about 10³ to 10¹⁵ CFUs, 10⁴ to 10¹⁰ CFUs, or about 10² to about 10⁸ CFUs. In some embodiments, the therapeutically effective amount of engineered yeast is from about 100 CFUs to about 10²⁰ CFUs. In some embodiments, the therapeutically effective amount of engineered yeast is from about 10³ to about 10¹⁵ CFUs. In some embodiments, the therapeutically effective amount of engineered yeast is from about 100 CFUs, about 10³ CFUs, or about 10⁴ CFUs to about 10⁸ CFUs, about 10¹⁰ CFUs, about 10¹⁵ CFUs, or about 10²⁰ CFUs. In some embodiments, the therapeutically effective amount of engineered yeast is any amount of CFU that falls within any of the above ranges.

### Methods of Repairing GI Epithelium

An engineered yeast may be used to promote healing and repair of GI epithelium, for example, as caused by any disease or condition such as IBD or IBS, through the production of trefoil factors (e.g., TFF1/2/3) or IGF-1.

Accordingly, in some embodiments, a method of promoting growth and repair in GI endothelium in a subject in need thereof is provided, the method comprising administering to the subject a therapeutically effective amount of an engineered yeast genetically modified to express a recombinant polypeptide comprising one or more of TFF1, TFF2, TFF3, or IGF-1 and synthetic signal peptide, thereby promoting growth and repair in GI endothelium. In some embodiments, the synthetic signal peptide comprises one or both of a) a pre-protein amino acid sequence of Formula II, Formula III, Formula IV, Formula V, Formula IX, Formula XIII or SEQ ID NO. 1, 2, 3, 4, 5, 6, 7, 8, 9, 10, 11, 12, 13, 14, 15, 16, 28, 31, 32, 33, 55, 70, 71, 72, or 73; and b) a pro-protein amino acid sequence of Formula VI, Formula VII, Formula VIII, Formula X, Formula XI, Formula XIV, Formula XV or SEQ ID NO. 17, 18, 19, 20, 21, 22, 23, 24, 25, 27, 29, 34, 35, 36, 37, 38, 56, 57, 58, 74, or 75. In some embodiments, the engineered yeast may be any strain as disclosed herein. In some embodiments, the engineered yeast is selected from the group comprising Kluyveromyces (e.g., K. lactis), Pichia (e.g., P. pastoris), Saccharomyces (e.g., S. cerevisiae, S. boulardii), Trichoderma (e.g., T. reesei, T. viride), and Aspergillus (e.g., A. niger).

In some embodiments, a method of promoting GI growth and repair is provided, the method comprising administering Kluyveromyces yeast (e.g., K. lactis), genetically modified with a nucleic acid encoding a recombinant polypeptide comprising TFF1, TFF2, TFF3, or IGF-1 and one or both of a) a pre-protein signal peptide comprising an amino acid sequence of Formula I or SEQ ID NO. 1 and b) a pro-protein signal peptide comprising an amino acid sequence of Formula VI or SEQ ID NO. 20 or 21, thereby promoting GI growth and repair.

In some embodiments, a method of promoting GI growth and repair is provided, the method comprising administering Pichia yeast (e.g., P. pastoris), genetically modified with a nucleic acid encoding a recombinant polypeptide comprising TFF1, TFF2, TFF3, or IGF-1 and one or both of a) a pre-protein signal peptide comprising an amino acid sequence of Formula II or SEQ ID NO. 2, 3, 4, 5, 6, or 7 and b) a pro-protein signal peptide comprising an amino acid sequence of Formula VI or SEQ ID NO. 17, 20 or 21, thereby promoting GI growth and repair.

In some embodiments, a method of promoting GI growth and repair is provided, the method comprising administering a Saccharomyces yeast (e.g., S. cerevisiae or S. boulardii), genetically modified with a nucleic acid encoding a recombinant polypeptide comprising TFF1, TFF2, TFF3, or IGF-1 and one or both of a) a pre-protein signal peptide comprising an amino acid sequence of Formula III, Formula IV, Formula V, or SEQ ID NO. 8, 9, 10, 11, 12, 13, 14, 15, or 16 and b) a pro-protein signal peptide comprising an amino acid sequence of Formula VI, Formula VII, Formula VIII, or SEQ ID NO. 18, 19, 20, 21, 22, 23, 24, 25, thereby promoting GI growth and repair.

In some embodiments, a method of promoting GI growth and repair is provided, the method comprising administering a Trichoderma yeast (e.g., T. reesei or T. viride), genetically modified with a nucleic acid encoding a recombinant polypeptide comprising TFF1, TFF2, TFF3, or IGF-1 and one or both of a) a pre-protein signal peptide comprising an amino acid sequence of Formula IX or SEQ ID NO. 31, 32, or 33 and b) a pro-protein signal peptide comprising an amino acid sequence of Formula X, Formula XI, or SEQ ID NO. 34, 35, 36, 37, or 38, thereby promoting GI growth and repair.

In some embodiments, a method of promoting GI growth and repair is provided, the method comprising administering an Aspergillus yeast (e.g., A. niger), genetically modified with a nucleic acid encoding a recombinant polypeptide comprising TFF1, TFF2, TFF3, or IGF-1 and one or both of a) a pre-protein signal peptide comprising an amino acid sequence of Formula XIII or SEQ ID NO. 70, 71, 72, or 73 and b) a pro-protein signal peptide comprising an amino acid sequence of Formula XIV, Formula XV, or SEQ ID NO. 74 or 75, thereby promoting GI growth and repair.

In any embodiment, growth and/or repair of GI epithelium may be in the context of a condition or disease such as short bowel syndrome, IBS, IBD, or any other disease where the GI epithelium is damaged or dysfunctional. In some embodiments, administering may be performed via any route. In some embodiments, the route of administration is oral or topical. The therapeutically effective amount of engineered yeast may be, for example, about 100 CFUs to 10²⁰ CFUs, about 10³ to 10¹⁵ CFUs, 10⁴ to 10¹⁰ CFUs, or about 10² to about 10⁸ CFUs. In some embodiments, the therapeutically effective amount of engineered yeast is from about 100 CFUs to about 10²⁰ CFUs. In some embodiments, the therapeutically effective amount of engineered yeast is from about 10³ to about 10¹⁵ CFUs. In some embodiments, the therapeutically effective amount of engineered yeast is from about 100 CFUs, about 10³ CFUs, or about 10⁴ CFUs to about 10⁸ CFUs, about 10¹⁰ CFUs, about 10¹⁵ CFUs, or about 10²⁰ CFUs. In some embodiments, the therapeutically effective amount of engineered yeast is any amount of CFU that falls within any of the above ranges.

### Methods of Treating Short Bowel Syndrome

An engineered yeast may be used to treat short bowel syndrome. Accordingly, in some embodiments, a method of treating short bowel syndrome in a subject in need thereof is provided, the method comprising administering to the subject a therapeutically effective amount of an engineered yeast genetically modified to express a recombinant polypeptide comprising IGF-1, GLP-2 or any synthetic analog or prodrug thereof and synthetic signal peptide. In some embodiments, the synthetic signal peptide comprises one or both of a) a pre-protein amino acid sequence of Formula II, Formula III, Formula IV, Formula V, Formula IX, Formula XIII or SEQ ID NO. 1, 2, 3, 4, 5, 6, 7, 8, 9, 10, 11, 12, 13, 14, 15, 16, 28, 31, 32, 33, 55, 70, 71, 72, or 73; and b) a pro-protein amino acid sequence of Formula VI, Formula VII, Formula VIII, Formula X, Formula XI, Formula XIV, Formula XV or SEQ ID NO. 17, 18, 19, 20, 21, 22, 23, 24, 25, 27, 29, 34, 35, 36, 37, 38, 56, 57, 58, 74, or 75. In some embodiments, the engineered yeast may be any strain as disclosed herein. In some embodiments, the engineered yeast is selected from the group comprising Kluyveromyces (e.g., K. lactis), Pichia (e.g., P. pastoris), Saccharomyces (e.g., S. cerevisiae, S. boulardii), Trichoderma (e.g., T. reesei, T. viride), and Aspergillus (e.g., A. niger).

In some embodiments, a method of treating short bowel syndrome is provided, the method comprising administering Kluyveromyces yeast (e.g., K. lactis), genetically modified with a nucleic acid encoding a recombinant polypeptide comprising IGF-1, GLP-2 or any synthetic analog or prodrug thereof and one or both of a) a pre-protein signal peptide comprising an amino acid sequence of Formula I or SEQ ID NO. 1 and b) a pro-protein signal peptide comprising an amino acid sequence of Formula VI or SEQ ID NO. 20, 21, thereby treating short bowel syndrome.

In some embodiments, a method treating short bowel syndrome is provided, the method comprising administering Pichia yeast (e.g., P. pastoris), genetically modified with a nucleic acid encoding a recombinant polypeptide comprising IGF-1 or an analog or prodrug thereof and one or both of a) a pre-protein signal peptide comprising an amino acid sequence of Formula II or SEQ ID NO. 2, 3, 4, 5, 6, or 7 and b) a pro-protein signal peptide comprising an amino acid sequence of Formula VI or SEQ ID NO. 17, 20, or 21, thereby treating short bowel syndrome.

In some embodiments, a method of treating short bowel syndrome is provided, the method comprising administering a Saccharomyces yeast (e.g., S. cerevisiae or S. boulardii), genetically modified with a nucleic acid encoding a recombinant polypeptide comprising IGF-1, GLP-2 or any synthetic analog or prodrug thereof and one or both of a) a pre-protein signal peptide comprising an amino acid sequence of Formula III, Formula IV, Formula V, or SEQ ID NO. 8, 9, 10, 11, 12, 13, 14, 15, or 16 and b) a pro-protein signal peptide comprising an amino acid sequence of Formula VI, Formula VII, Formula VIII, or SEQ ID NO. 18, 19, 20, 21, 22, 23, 24, 25, thereby treating short bowel syndrome.

In some embodiments, a method of treating short bowel syndrome is provided, the method comprising administering a Trichoderma yeast (e.g., T. reesei or T. viride), genetically modified with a nucleic acid encoding a recombinant polypeptide comprising IGF-1, GLP-2 or any synthetic analog or prodrug thereof and one or both of a) a pre-protein signal peptide comprising an amino acid sequence of Formula IX or SEQ ID NO. 31, 32, or 33 and b) a pro-protein signal peptide comprising an amino acid sequence of Formula X, Formula XI, or SEQ ID NO. 34, 35, 36, 37, or 38, thereby treating short bowel syndrome.

In some embodiments, a method of treating short bowel syndrome is provided, the method comprising administering an Aspergillus yeast (e.g., A. niger), genetically modified with a nucleic acid encoding a recombinant polypeptide comprising IGF-1, GLP-2 or any synthetic analog or prodrug thereof and one or both of a) a pre-protein signal peptide comprising an amino acid sequence of Formula XIII or SEQ ID NO. 70, 71, 72, or 73 and b) a pro-protein signal peptide comprising an amino acid sequence of Formula XIV, Formula XV, or SEQ ID NO. 74 or 75, thereby treating short bowel syndrome.

In some embodiments, administering may be performed via any route. In some embodiments, the route of administration is oral or topical. The therapeutically effective amount of engineered yeast may be, for example, about 100 CFUs to 10²⁰ CFUs, about 10³ to 10¹⁵ CFUs, 10⁴ to 10¹⁰ CFUs, or about 10² to about 10⁸ CFUs. In some embodiments, the therapeutically effective amount of engineered yeast is from about 100 CFUs to about 10²⁰ CFUs. In some embodiments, the therapeutically effective amount of engineered yeast is from about 10³ to about 10¹⁵ CFUs. In some embodiments, the therapeutically effective amount of engineered yeast is from about 100 CFUs, about 10³ CFUs, or about 10⁴ CFUs to about 10⁸ CFUs, about 10¹⁰ CFUs, about 10¹⁵ CFUs, or about 10²⁰ CFUs. In some embodiments, the therapeutically effective amount of engineered yeast is any amount of CFU that falls within any of the above ranges.

### Methods of Treating Trehalose Sensitivity

Trehalase deficiency is a metabolic condition where the body lacks the enzyme trehalase and is therefore unable to convert trehalose into glucose. Accordingly, in some embodiments, a method of treating a trehalase deficiency in a subject in need thereof is provided, the method comprising administering to the subject a therapeutically effective amount of an engineered yeast genetically modified to express a recombinant polypeptide comprising trehalase (or a pro-drug or active variant thereof) and a synthetic signal peptide, thereby treating the deficiency. In some embodiments, the synthetic signal peptide comprises one or both of a) an pre-protein amino acid sequence of Formula II, Formula III, Formula IV, Formula V, Formula IX, Formula XIII or SEQ ID NO. 1, 2, 3, 4, 5, 6, 7, 8, 9, 10, 11, 12, 13, 14, 15, 16, 28, 31, 32, 33, 55, 70, 71, 72, or 73; and b) a pro-protein amino acid sequence of Formula VI, Formula VII, Formula VIII, Formula X, Formula XI, Formula XIV, Formula XV or SEQ ID NO. 17, 18, 19, 20, 21, 22, 23, 24, 25, 27, 29, 34, 35, 36, 37, 38, 56, 57, 58, 74, or 75. In some embodiments, the engineered yeast may be any strain as disclosed herein. In some embodiments, the engineered yeast is selected from the group comprising Kluyveromyces (e.g., K. lactis), Pichia (e.g., P. pastoris), Saccharomyces (e.g., S. cerevisiae, S. boulardii), Trichoderma (e.g., T. reesei, T. viride), and Aspergillus (e.g., A. niger).

In some embodiments, a method for treating trehalose sensitivity is provided, the method comprising administering to a subject in need thereof Kluyveromyces yeast (e.g., K. lactis), genetically modified with a nucleic acid encoding a recombinant polypeptide comprising trehalase and one or both of a) a pre-protein signal peptide comprising an amino acid sequence of Formula I or SEQ ID NO. 1 and b) a pro-protein signal peptide comprising an amino acid sequence of Formula VI or SEQ ID NO. 20 or 21, thereby treating the trehalose sensitivity.

In some embodiments, a method for treating trehalose sensitivity is provided, the method comprising administering to a subject in need thereof Pichia yeast (e.g., P. pastoris), genetically modified with a nucleic acid encoding a recombinant polypeptide comprising trehalase and one or both of a) a pre-protein signal peptide comprising an amino acid sequence of Formula II or SEQ ID NO. 2, 3, 4, 5, 6, or 7 and b) a pro-protein signal peptide comprising an amino acid sequence of Formula VI or SEQ ID NO. 17, 20 or 21, thereby treating the trehalose sensitivity.

In some embodiments, a method of treating trehalose sensitivity is provided, the method comprising administering to a subject in need thereof a Saccharomyces yeast (e.g., S. cerevisiae or S. boulardii), genetically modified with a nucleic acid encoding a recombinant polypeptide comprising trehalase and one or both of a) a pre-protein signal peptide comprising an amino acid sequence of Formula III, Formula IV, Formula V, or SEQ ID NO. 8, 9, 10, 11, 12, 13, 14, 15, or 16 and b) a pro-protein signal peptide comprising an amino acid sequence of Formula VI, Formula VII, Formula VIII, or SEQ ID NO. 18, 19, 20, 21, 22, 23, 24, or 25, thereby treating the trehalose sensitivity.

In some embodiments, a method of treating trehalose sensitivity is provided, the method comprising administering to a subject in need thereof a Trichoderma yeast (e.g., T. reesei or T. viride), genetically modified with a nucleic acid encoding a recombinant polypeptide comprising trehalase and one or both of a) a pre-protein signal peptide comprising an amino acid sequence of Formula IX or SEQ ID NO. 31, 32, or 33 and b) a pro-protein signal peptide comprising an amino acid sequence of Formula X, Formula XI, or SEQ ID NO. 34, 35, 36, 37, or 38, thereby treating the trehalose sensitivity.

In some embodiments, a method of treating trehalose sensitivity is provided, the method comprising administering to a subject in need thereof an Aspergillus yeast (e.g., A. niger), genetically modified with a nucleic acid encoding a recombinant polypeptide comprising trehalase and one or both of a) a pre-protein signal peptide comprising an amino acid sequence of Formula XIII or SEQ ID NO. 70, 71, 72, or 73 and b) a pro-protein signal peptide comprising an amino acid sequence of Formula XIV, Formula XV, or SEQ ID NO. 74 or 75, thereby treating the trehalose sensitivity.

In some embodiments, administering may be performed via any route. In some embodiments, the route of administration is oral or topical. The therapeutically effective amount of engineered yeast may be, for example, about 100 CFUs to 10²⁰ CFUs, about 10³ to 10¹⁵ CFUs, 10⁴ to 10¹⁰ CFUs, or about 10² to about 10⁸ CFUs. In some embodiments, the therapeutically effective amount of engineered yeast is from about 100 CFUs to about 10²⁰ CFUs. In some embodiments, the therapeutically effective amount of engineered yeast is from about 10³ to about 10¹⁵ CFUs. In some embodiments, the therapeutically effective amount of engineered yeast is from about 100 CFUs, about 10³ CFUs, or about 10⁴ CFUs to about 10⁸ CFUs, about 10¹⁰ CFUs, about 10¹⁵ CFUs, or about 10²⁰ CFUs. In some embodiments, the therapeutically effective amount of engineered yeast is any amount of CFU that falls within any of the above ranges.

### Methods of Treating Pernicious Anemia

Pernicious anemia is a rare blood disorder characterized by the inability of the body to properly utilize vitamin B12, resulting from the lack of the gastric protein intrinsic factor, without which B12 cannot be absorbed. Accordingly, in some embodiments, a method of treating pernicious anemia in a subject in need thereof is provided, the method comprising administering to the subject a therapeutically effective amount of an engineered yeast genetically modified to express a recombinant polypeptide comprising intrinsic factor (or a pro-drug or active variant thereof) and a synthetic signal peptide. In some embodiments, the synthetic signal peptide comprises one or both of a) an pre-protein amino acid sequence of Formula II, Formula III, Formula IV, Formula V, Formula IX, Formula XIII or SEQ ID NO. 1, 2, 3, 4, 5, 6, 7, 8, 9, 10, 11, 12, 13, 14, 15, 16, 28, 31, 32, 33, 55, 70, 71, 72, or 73; and b) a pro-protein amino acid sequence of Formula VI, Formula VII, Formula VIII, Formula X, Formula XI, Formula XIV, Formula XV or SEQ ID NO. 17, 18, 19, 20, 21, 22, 23, 24, 25, 27, 29, 34, 35, 36, 37, 38, 56, 57, 58, 74, or 75. In some embodiments, the engineered yeast may be any strain as disclosed herein. In some embodiments, the engineered yeast is selected from the group comprising Kluyveromyces (e.g., K. lactis), Pichia (e.g., P. pastoris), Saccharomyces (e.g., S. cerevisiae, S. boulardii), Trichoderma (e.g., T. reesei, T. viride), and Aspergillus (e.g., A. niger).

In some embodiments, a method of treating pernicious anemia is provided, the method comprising administering to a subject in need thereof Kluyveromyces yeast (e.g., K. lactis), genetically modified with a nucleic acid encoding a recombinant polypeptide comprising intrinsic factor and one or both of a) a pre-protein signal peptide comprising an amino acid sequence of Formula I or SEQ ID NO. 1 and b) a pro-protein signal peptide comprising an amino acid sequence of Formula VI or SEQ ID NO. 20 or 21, thereby treating pernicious anemia.

In some embodiments, a method of treating pernicious anemia is provided, the method comprising administering to a subject in need thereof Pichia yeast (e.g., P. pastoris), genetically modified with a nucleic acid encoding a recombinant polypeptide comprising intrinsic factor and one or both of a) a pre-protein signal peptide comprising an amino acid sequence of Formula II or SEQ ID NO. 2, 3, 4, 5, 6, or 7 and b) a pro-protein signal peptide comprising an amino acid sequence of Formula VI or SEQ ID NO. 17, 20 or 21, thereby treating pernicious anemia.

In some embodiments, a method of treating pernicious anemia is provided, the method comprising administering to a subject in need thereof a Saccharomyces yeast (e.g., S. cerevisiae or S. boulardii), genetically modified with a nucleic acid encoding a recombinant polypeptide comprising intrinsic factor and one or both of a) a pre-protein signal peptide comprising an amino acid sequence of Formula III, Formula IV, Formula V, or SEQ ID NO. 8, 9, 10, 11, 12, 13, 14, 15, or 16 and b) a pro-protein signal peptide comprising an amino acid sequence of Formula VI, Formula VII, Formula VIII, or SEQ ID NO. 18, 19, 20, 21, 22, 23, 24, or 25, thereby treating pernicious anemia.

In some embodiments, a method of treating pernicious anemia is provided, the method comprising administering to a subject in need thereof a Trichoderma yeast (e.g., T. reesei or T. viride), genetically modified with a nucleic acid encoding a recombinant polypeptide comprising intrinsic factor and one or both of a) a pre-protein signal peptide comprising an amino acid sequence of Formula IX or SEQ ID NO. 31, 32, or 33 and b) a pro-protein signal peptide comprising an amino acid sequence of Formula X, Formula XI, or SEQ ID NO. 34, 35, 36, 37, or 38, thereby treating pernicious anemia.

In some embodiments, a method of treating pernicious anemia is provided, the method comprising administering to a subject in need thereof an Aspergillus yeast (e.g., A. niger), genetically modified with a nucleic acid encoding a recombinant polypeptide comprising intrinsic factor and one or both of a) a pre-protein signal peptide comprising an amino acid sequence of Formula XIII or SEQ ID NO. 70, 71, 72, or 73 and b) a pro-protein signal peptide comprising an amino acid sequence of Formula XIV, Formula XV, or SEQ ID NO. 74 or 75, thereby treating pernicious anemia.

In some embodiments, administering may be performed via any route. In some embodiments, the route of administration is oral or topical. The therapeutically effective amount of engineered yeast may be, for example, about 100 CFUs to 10²⁰ CFUs, about 10³ to 10¹⁵ CFUs, 10⁴ to 10¹⁰ CFUs, or about 10² to about 10⁸ CFUs. In some embodiments, the therapeutically effective amount of engineered yeast is from about 100 CFUs to about 10²⁰ CFUs. In some embodiments, the therapeutically effective amount of engineered yeast is from about 10³ to about 10¹⁵ CFUs. In some embodiments, the therapeutically effective amount of engineered yeast is from about 100 CFUs, about 10³ CFUs, or about 10⁴ CFUs to about 10⁸ CFUs, about 10¹⁰ CFUs, about 10¹⁵ CFUs, or about 10²⁰ CFUs. In some embodiments, the therapeutically effective amount of engineered yeast is any amount of CFU that falls within any of the above ranges.

### Method of Reducing Inflammation

An engineered yeast may be used to produce pro-repair cytokines such as IL-10, IL-22, and/or TGFβ, which may be suitable for treating a variety of diseases and conditions. Further, engineered yeast may be used to produce anti-TNFα antibodies or fragments of anti-TNFα antibodies. Oral administration of IL-10, IL-22, TGFβ and/or anti-TNFα antibodies or fragments thereof may be beneficial for treating and repairing damage caused by inflammatory GI conditions, such as IBS, IBD, and the like. In some embodiments, an engineered yeast genetically modified to express IL-10 may be orally administered to a subject to treat Crohn's disease or inhibit tumor metastasis. Accordingly, in some embodiments, a method of treating an inflammatory condition in a subject in need thereof is provided, the method comprising administering to the subject a therapeutically effective amount of an engineered yeast genetically modified to express a recombinant polypeptide comprising one or more of IL-10, IL-22, TGFβ, and anti-TNFα antibodies or fragments thereof, or an analog or prodrug thereof and synthetic signal peptide. In some embodiments, the synthetic signal peptide comprises one or both of a) an pre-protein amino acid sequence of Formula II, Formula III, Formula IV, Formula V, Formula IX, Formula XIII or SEQ ID NO. 1, 2, 3, 4, 5, 6, 7, 8, 9, 10, 11, 12, 13, 14, 15, 16, 28, 31, 32, 33, 55, 70, 71, 72, or 73; and b) a pro-protein amino acid sequence of Formula VI, Formula VII, Formula VIII, Formula X, Formula XI, Formula XIV, Formula XV or SEQ ID NO. 17, 18, 19, 20, 21, 22, 23, 24, 25, 27, 29, 34, 35, 36, 37, 38, 56, 57, 58, 74, or 75. In some embodiments, the engineered yeast may be any strain as disclosed herein. In some embodiments, the engineered yeast is selected from the group comprising Kluyveromyces (e.g., K. lactis), Pichia (e.g., P. pastoris), Saccharomyces (e.g., S. cerevisiae, S. boulardii), Trichoderma (e.g., T. reesei, T. viride), and Aspergillus (e.g., A. niger).

In some embodiments, a method of treating inflammation is provided, the method comprising administering Kluyveromyces yeast (e.g., K. lactis), genetically modified with a nucleic acid encoding a recombinant polypeptide comprising one or more of IL-10, IL-22, TGFβ, and anti-TNFα antibodies or fragments thereof, or an analog or prodrug thereof and one or both of a) a pre-protein signal peptide comprising an amino acid sequence of Formula I or SEQ ID NO. 1 and b) a pro-protein signal peptide comprising an amino acid sequence of Formula VI or SEQ ID NO. 20 or 21, thereby treating the inflammation.

In some embodiments, a method of treating inflammation is provided, the method comprising administering Pichia yeast (e.g., P. pastoris), genetically modified with a nucleic acid encoding a recombinant polypeptide comprising one or more of IL-10, IL-22, TGFβ, and anti-TNFα antibodies or fragments thereof, or an analog or prodrug thereof and one or both of a) a pre-protein signal peptide comprising an amino acid sequence of Formula II or SEQ ID NO. 2, 3, 4, 5, 6, or 7 and b) a pro-protein signal peptide comprising an amino acid sequence of Formula VI or SEQ ID NO. 17, 20, or 21, thereby treating the inflammation.

In some embodiments, a method of treating inflammation is provided, the method comprising administering a Saccharomyces yeast (e.g., S. cerevisiae or S. boulardii), genetically modified with a nucleic acid encoding a recombinant polypeptide comprising one or more of IL-10, IL-22, TGFβ, and anti-TNFα antibodies or fragments thereof, or an analog or prodrug thereof and one or both of a) a pre-protein signal peptide comprising an amino acid sequence of Formula III, Formula IV, Formula V, or SEQ ID NO. 8, 9, 10, 11, 12, 13, 14, 15, or 16 and b) a pro-protein signal peptide comprising an amino acid sequence of Formula VI, Formula VII, Formula VIII, or SEQ ID NO. 18, 19, 20, 21, 22, 23, 24, or 25, thereby treating the inflammation.

In some embodiments, a method of treating inflammation is provided, the method comprising administering a Trichoderma yeast (e.g., T. reesei or T. viride), genetically modified with a nucleic acid encoding a recombinant polypeptide comprising one or more of IL-10, IL-22, TGFβ, and anti-TNFα antibodies or fragments thereof, or an analog or prodrug thereof and one or both of a) a pre-protein signal peptide comprising an amino acid sequence of Formula IX or SEQ ID NO. 31, 32, or 33 and b) a pro-protein signal peptide comprising an amino acid sequence of Formula X, Formula XI, or SEQ ID NO. 34, 35, 36, 37, or 38, thereby treating the inflammation.

In some embodiments, a method of treating inflammation is provided, the method comprising administering an Aspergillus yeast (e.g., A. niger), genetically modified with a nucleic acid encoding a recombinant polypeptide comprising one or more of IL-10, IL-22, TGFβ, and anti-TNFα antibodies or fragments thereof, or an analog or prodrug thereof and one or both of a) a pre-protein signal peptide comprising an amino acid sequence of Formula XIII or SEQ ID NO. 70, 71, 72, or 73 and b) a pro-protein signal peptide comprising an amino acid sequence of Formula XIV, Formula XV, or SEQ ID NO. 74 or 75, thereby treating the inflammation.

In some embodiments, administering may be performed via any route. In some embodiments, the route of administration is oral or topical. The therapeutically effective amount of engineered yeast may be, for example, about 100 CFUs to 10²⁰ CFUs, about 10³ to 10¹⁵ CFUs, 10⁴ to 10¹⁰ CFUs, or about 10² to about 10⁸ CFUs. In some embodiments, the therapeutically effective amount of engineered yeast is from about 100 CFUs to about 10²⁰ CFUs. In some embodiments, the therapeutically effective amount of engineered yeast is from about 10³ to about 10¹⁵ CFUs. In some embodiments, the therapeutically effective amount of engineered yeast is from about 100 CFUs, about 10³ CFUs, or about 10⁴ CFUs to about 10⁸ CFUs, about 10¹⁰ CFUs, about 10¹⁵ CFUs, or about 10²⁰ CFUs. In some embodiments, the therapeutically effective amount of engineered yeast is any amount of CFU that falls within any of the above ranges.

### Method of Treating Cancer

An engineered yeast may be used for treating a variety of cancers, for example, but not limited to, cancers of the GI tract. Accordingly, in some embodiments, a method of treating cancer in a subject in need thereof is provided, the method comprising administering to the subject a therapeutically effective amount of an engineered yeast genetically modified to express a recombinant polypeptide comprising one or more of an anti-cancer therapeutic and synthetic signal peptide. In some embodiments, the synthetic signal peptide comprises one or both of a) an pre-protein amino acid sequence of Formula II, Formula III, Formula IV, Formula V, Formula IX, Formula XIII or SEQ ID NO. 1, 2, 3, 4, 5, 6, 7, 8, 9, 10, 11, 12, 13, 14, 15, 16, 28, 31, 32, 33, 55, 70, 71, 72, or 73; and b) a pro-protein amino acid sequence of Formula VI, Formula VII, Formula VIII, Formula X, Formula XI, Formula XIV, Formula XV or SEQ ID NO. 17, 18, 19, 20, 21, 22, 23, 24, 25, 27, 29, 34, 35, 36, 37, 38, 56, 57, 58, 74, or 75. In some embodiments, the engineered yeast may be any strain as disclosed herein. In some embodiments, the engineered yeast is selected from the group comprising Kluyveromyces (e.g., K. lactis), Pichia (e.g., P. pastoris), Saccharomyces (e.g., S. cerevisiae, S. boulardii), Trichoderma (e.g., T. reesei, T. viride), and Aspergillus (e.g., A. niger).

In some embodiments, a method of treating cancer is provided, the method comprising administering Kluyveromyces yeast (e.g., K. lactis), genetically modified with a nucleic acid encoding a recombinant polypeptide comprising one or more of an anti-cancer therapeutic and one or both of a) a pre-protein signal peptide comprising an amino acid sequence of Formula I or SEQ ID NO. 1 and b) a pro-protein signal peptide comprising an amino acid sequence of Formula VI or SEQ ID NO. 20 or 21, thereby treating the inflammation.

In some embodiments, a method of treating cancer is provided, the method comprising administering Pichia yeast (e.g., P. pastoris), genetically modified with a nucleic acid encoding a recombinant polypeptide comprising one or more of an anti-cancer therapeutic and one or both of a) a pre-protein signal peptide comprising an amino acid sequence of Formula II or SEQ ID NO. 2, 3, 4, 5, 6, or 7 and b) a pro-protein signal peptide comprising an amino acid sequence of Formula VI or SEQ ID NO. 17, 20, or 21, thereby treating the inflammation.

In some embodiments, a method of treating cancer is provided, the method comprising administering a Saccharomyces yeast (e.g., S. cerevisiae or S. boulardii), genetically modified with a nucleic acid encoding a recombinant polypeptide comprising one or more an anti-cancer therapeutic and one or both of a) a pre-protein signal peptide comprising an amino acid sequence of Formula III, Formula IV, Formula V, or SEQ ID NO. 8, 9, 10, 11, 12, 13, 14, 15, or 16 and b) a pro-protein signal peptide comprising an amino acid sequence of Formula VI, Formula VII, Formula VIII, or SEQ ID NO. 18, 19, 20, 21, 22, 23, 24, or 25, thereby treating the inflammation.

In some embodiments, a method of treating cancer is provided, the method comprising administering a Trichoderma yeast (e.g., T. reesei or T. viride), genetically modified with a nucleic acid encoding a recombinant polypeptide comprising one or more of an anti-cancer therapeutic and one or both of a) a pre-protein signal peptide comprising an amino acid sequence of Formula IX or SEQ ID NO. 31, 32, or 33 and b) a pro-protein signal peptide comprising an amino acid sequence of Formula X, Formula XI, or SEQ ID NO. 34, 35, 36, 37, or 38, thereby treating the inflammation.

In some embodiments, a method of treating cancer is provided, the method comprising administering an Aspergillus yeast (e.g., A. niger), genetically modified with a nucleic acid encoding a recombinant polypeptide comprising one or more of an anti-cancer therapeutic and one or both of a) a pre-protein signal peptide comprising an amino acid sequence of Formula XIII or SEQ ID NO. 70, 71, 72, or 73 and b) a pro-protein signal peptide comprising an amino acid sequence of Formula XIV, Formula XV, or SEQ ID NO. 74 or 75, thereby treating the inflammation.

In some embodiments, administering may be performed via any route. In some embodiments, the route of administration is oral or topical. The therapeutically effective amount of engineered yeast may be, for example, about 100 CFUs to 10²⁰ CFUs, about 10³ to 10¹⁵ CFUs, 10⁴ to 10¹⁰ CFUs, or about 10² to about 10⁸ CFUs. In some embodiments, the therapeutically effective amount of engineered yeast is from about 100 CFUs to about 10²⁰ CFUs. In some embodiments, the therapeutically effective amount of engineered yeast is from about 10³ to about 10¹⁵ CFUs. In some embodiments, the therapeutically effective amount of engineered yeast is from about 100 CFUs, about 10³ CFUs, or about 10⁴ CFUs to about 10⁸ CFUs, about 10¹⁰ CFUs, about 10¹⁵ CFUs, or about 10²⁰ CFUs. In some embodiments, the therapeutically effective amount of engineered yeast is any amount of CFU that falls within any of the above ranges.

### Method of Promoting Appetite Suppression

An engineered yeast may be used to induce the release of the peptide hormone cholecystokinin (CCK, also known as pancreozymin), which has important roles in digestion and satiety. Oral administration of luminal CCK-releasing factor (LCRF) may be beneficial for promoting appetite suppression, delaying of gastric emptying, and/or inducing pancreatic secretion. Other proteins that exhibit these same functions include casein and soy proteins. Thus, administration of LCRF, casein, and/or soy proteins may be useful in the treatment of several digestive disorders and obesity through i) the suppression of appetite and ii) the promotion of digestion. In some embodiments, an engineered yeast genetically modified to express LCRF, casein, and/or soy proteins may be orally administered to a subject to promote appetite suppression. Accordingly, in some embodiments, a method of promoting appetite suppression in a subject in need thereof is provided, the method comprising administering to the subject a therapeutically effective amount of an engineered yeast genetically modified to express a recombinant polypeptide comprising LCRF and synthetic signal peptide. In some embodiments, the recombinant polypeptide comprises casein. In some embodiments, the recombinant polypeptide comprises soy proteins. In some embodiments, the synthetic signal peptide comprises one or both of a) an pre-protein amino acid sequence of Formula II, Formula III, Formula IV, Formula V, Formula IX, Formula XIII or SEQ ID NO. 1, 2, 3, 4, 5, 6, 7, 8, 9, 10, 11, 12, 13, 14, 15, 16, 28, 31, 32, 33, 55, 70, 71, 72, or 73; and b) a pro-protein amino acid sequence of Formula VI, Formula VII, Formula VIII, Formula X, Formula XI, Formula XIV, Formula XV or SEQ ID NO. 17, 18, 19, 20, 21, 22, 23, 24, 25, 27, 29, 34, 35, 36, 37, 38, 56, 57, 58, 74, or 75. In some embodiments, the engineered yeast may be any strain as disclosed herein. In some embodiments, the engineered yeast is selected from the group comprising Kluyveromyces (e.g., K. lactis), Pichia (e.g., P. pastoris), Saccharomyces (e.g., S. cerevisiae, S. boulardii), Trichoderma (e.g., T. reesei, T. viride), and Aspergillus (e.g., A. niger).

In some embodiments, a method of promoting appetite suppression is provided, the method comprising administering Kluyveromyces yeast (e.g., K. lactis), genetically modified with a nucleic acid encoding a recombinant polypeptide comprising LCRF and one or both of a) a pre-protein signal peptide comprising an amino acid sequence of Formula I or SEQ ID NO. 1 and b) a pro-protein signal peptide comprising an amino acid sequence of Formula VI or SEQ ID NO. 20 or 21, thereby promoting appetite suppression.

In some embodiments, a method of promoting appetite suppression is provided, the method comprising administering Pichia yeast (e.g., P. pastoris), genetically modified with a nucleic acid encoding a recombinant polypeptide comprising LCRF and one or both of a) a pre-protein signal peptide comprising an amino acid sequence of Formula II or SEQ ID NO. 2, 3, 4, 5, 6, or 7 and b) a pro-protein signal peptide comprising an amino acid sequence of Formula VI or SEQ ID NO. 17, 20, or 21, thereby promoting appetite suppression.

In some embodiments, a method of promoting appetite suppression is provided, the method comprising administering a Saccharomyces yeast (e.g., S. cerevisiae or S. boulardii), genetically modified with a nucleic acid encoding a recombinant polypeptide comprising LCRF and one or both of a) a pre-protein signal peptide comprising an amino acid sequence of Formula III, Formula IV, Formula V, or SEQ ID NO. 8, 9, 10, 11, 12, 13, 14, 15, or 16 and b) apro-protein signal peptide comprising an amino acid sequence of Formula VI, Formula VII, Formula VIII, or SEQ ID NO. 18, 19, 20, 21, 22, 23, 24, or 25, thereby promoting appetite suppression.

In some embodiments, a method of promoting appetite suppression is provided, the method comprising administering a Trichoderma yeast (e.g., T. reesei or T. viride), genetically modified with a nucleic acid encoding a recombinant polypeptide comprising LCRF and one or both of a) a pre-protein signal peptide comprising an amino acid sequence of Formula IX or SEQ ID NO. 31, 32, or 33 and b) a pro-protein signal peptide comprising an amino acid sequence of Formula X, Formula XI, or SEQ ID NO. 34, 35, 36, 37, or 38, thereby promoting appetite suppression.

In some embodiments, a method of promoting appetite suppression is provided, the method comprising administering an Aspergillus yeast (e.g., A. niger), genetically modified with a nucleic acid encoding a recombinant polypeptide comprising LCRF and one or both of a) a pre-protein signal peptide comprising an amino acid sequence of Formula XIII or SEQ ID NO. 70, 71, 72, or 73 and b) a pro-protein signal peptide comprising an amino acid sequence of Formula XIV, Formula XV, or SEQ ID NO. 74 or 75, thereby promoting appetite suppression.

In some embodiments, administering may be performed via any route. In some embodiments, the route of administration is oral or topical. The therapeutically effective amount of engineered yeast may be, for example, about 100 CFUs to 10²⁰ CFUs, about 10³ to 10¹⁵ CFUs, 10⁴ to 10¹⁰ CFUs, or about 10² to about 10⁸ CFUs. In some embodiments, the therapeutically effective amount of engineered yeast is from about 100 CFUs to about 10²⁰ CFUs. In some embodiments, the therapeutically effective amount of engineered yeast is from about 10³ to about 10¹⁵ CFUs. In some embodiments, the therapeutically effective amount of engineered yeast is from about 100 CFUs, about 10³ CFUs, or about 10⁴ CFUs to about 10⁸ CFUs, about 10¹⁰ CFUs, about 10¹⁵ CFUs, or about 10²⁰ CFUs. In some embodiments, the therapeutically effective amount of engineered yeast is any amount of CFU that falls within any of the above ranges.

### Method of Delaying of Gastric Emptying

An engineered yeast may be used to induce the release of the peptide hormone cholecystokinin (CCK, also known as pancreozymin), which has important roles in digestion and satiety. Oral administration of luminal CCK-releasing factor (LCRF) may be beneficial for promoting appetite suppression, delaying of gastric emptying, and/or inducing pancreatic secretion. Other proteins that exhibit these same functions include casein and soy proteins. Thus, administration of LCRF, casein, and/or soy proteins may be useful in the treatment of several digestive disorders and obesity through i) the suppression of appetite and ii) the promotion of digestion. In some embodiments, an engineered yeast genetically modified to express LCRF, casein, and/or soy proteins may be orally administered to a subject to promote appetite suppression. Accordingly, in some embodiments, a method of delaying of gastric emptying in a subject in need thereof is provided, the method comprising administering to the subject a therapeutically effective amount of an engineered yeast genetically modified to express a recombinant polypeptide comprising LCRF and synthetic signal peptide. In some embodiments, the recombinant polypeptide comprises casein. In some embodiments, the recombinant polypeptide comprises soy proteins. In some embodiments, the synthetic signal peptide comprises one or both of a) an pre-protein amino acid sequence of Formula II, Formula III, Formula IV, Formula V, Formula IX, Formula XIII or SEQ ID NO. 1, 2, 3, 4, 5, 6, 7, 8, 9, 10, 11, 12, 13, 14, 15, 16, 28, 31, 32, 33, 55, 70, 71, 72, or 73; and b) a pro-protein amino acid sequence of Formula VI, Formula VII, Formula VIII, Formula X, Formula XI, Formula XIV, Formula XV or SEQ ID NO. 17, 18, 19, 20, 21, 22, 23, 24, 25, 27, 29, 34, 35, 36, 37, 38, 56, 57, 58, 74, or 75. In some embodiments, the engineered yeast may be any strain as disclosed herein. In some embodiments, the engineered yeast is selected from the group comprising Kluyveromyces (e.g., K. lactis), Pichia (e.g., P. pastoris), Saccharomyces (e.g., S. cerevisiae, S. boulardii), Trichoderma (e.g., T. reesei, T. viride), and Aspergillus (e.g., A. niger).

In some embodiments, a method of delaying of gastric emptying is provided, the method comprising administering Kluyveromyces yeast (e.g., K. lactis), genetically modified with a nucleic acid encoding a recombinant polypeptide comprising LCRF and one or both of a) a pre-protein signal peptide comprising an amino acid sequence of Formula I or SEQ ID NO. 1 and b) a pro-protein signal peptide comprising an amino acid sequence of Formula VI or SEQ ID NO. 20 or 21, thereby delaying gastric emptying.

In some embodiments, a method of delaying of gastric emptying is provided, the method comprising administering Pichia yeast (e.g., P. pastoris), genetically modified with a nucleic acid encoding a recombinant polypeptide comprising LCRF and one or both of a) a pre-protein signal peptide comprising an amino acid sequence of Formula II or SEQ ID NO. 2, 3, 4, 5, 6, or 7 and b) a pro-protein signal peptide comprising an amino acid sequence of Formula VI or SEQ ID NO. 17, 20, or 21, thereby delaying gastric emptying.

In some embodiments, a method of delaying of gastric emptying is provided, the method comprising administering a Saccharomyces yeast (e.g., S. cerevisiae or S. boulardii), genetically modified with a nucleic acid encoding a recombinant polypeptide comprising LCRF and one or both of a) a pre-protein signal peptide comprising an amino acid sequence of Formula III, Formula IV, Formula V, or SEQ ID NO. 8, 9, 10, 11, 12, 13, 14, 15, or 16 and b) apro-protein signal peptide comprising an amino acid sequence of Formula VI, Formula VII, Formula VIII, or SEQ ID NO. 18, 19, 20, 21, 22, 23, 24, or 25, thereby delaying gastric emptying.

In some embodiments, a method of delaying gastric emptying is provided, the method comprising administering a Trichoderma yeast (e.g., T. reesei or T. viride), genetically modified with a nucleic acid encoding a recombinant polypeptide comprising LCRF and one or both of a) a pre-protein signal peptide comprising an amino acid sequence of Formula IX or SEQ ID NO. 31, 32, or 33 and b) a pro-protein signal peptide comprising an amino acid sequence of Formula X, Formula XI, or SEQ ID NO. 34, 35, 36, 37, or 38, thereby delaying gastric emptying.

In some embodiments, a method of delaying of gastric emptying is provided, the method comprising administering an Aspergillus yeast (e.g., A. niger), genetically modified with a nucleic acid encoding a recombinant polypeptide comprising LCRF and one or both of a) a pre-protein signal peptide comprising an amino acid sequence of Formula XIII or SEQ ID NO. 70, 71, 72, or 73 and b) a pro-protein signal peptide comprising an amino acid sequence of Formula XIV, Formula XV, or SEQ ID NO. 74 or 75, thereby delaying gastric emptying.

In some embodiments, administering may be performed via any route. In some embodiments, the route of administration is oral or topical. The therapeutically effective amount of engineered yeast may be, for example, about 100 CFUs to 10²⁰ CFUs, about 10³ to 10¹⁵ CFUs, 10⁴ to 10¹⁰ CFUs, or about 10² to about 10⁸ CFUs. In some embodiments, the therapeutically effective amount of engineered yeast is from about 100 CFUs to about 10²⁰ CFUs. In some embodiments, the therapeutically effective amount of engineered yeast is from about 10³ to about 10¹⁵ CFUs. In some embodiments, the therapeutically effective amount of engineered yeast is from about 100 CFUs, about 10³ CFUs, or about 10⁴ CFUs to about 10⁸ CFUs, about 10¹⁰ CFUs, about 10¹⁵ CFUs, or about 10²⁰ CFUs. In some embodiments, the therapeutically effective amount of engineered yeast is any amount of CFU that falls within any of the above ranges.

### Method of Inducing Pancreatic Secretion

An engineered yeast may be used to induce the release of the peptide hormone cholecystokinin (CCK, also known as pancreozymin), which has important roles in digestion and satiety. Oral administration of luminal CCK-releasing factor (LCRF) may be beneficial for promoting appetite suppression, delaying of gastric emptying, and/or inducing pancreatic secretion. Other proteins that exhibit these same functions include casein and soy proteins. Thus, administration of LCRF, casein, and/or soy proteins may be useful in the treatment of several digestive disorders and obesity through i) the suppression of appetite and ii) the promotion of digestion. In some embodiments, an engineered yeast genetically modified to express LCRF, casein, and/or soy proteins may be orally administered to a subject to promote appetite suppression. Accordingly, in some embodiments, a method of inducing pancreatic secretion in a subject in need thereof is provided, the method comprising administering to the subject a therapeutically effective amount of an engineered yeast genetically modified to express a recombinant polypeptide comprising LCRF and synthetic signal peptide. In some embodiments, the recombinant polypeptide comprises casein. In some embodiments, the recombinant polypeptide comprises soy proteins. In some embodiments, the synthetic signal peptide comprises one or both of a) an pre-protein amino acid sequence of Formula II, Formula III, Formula IV, Formula V, Formula IX, Formula XIII or SEQ ID NO. 1, 2, 3, 4, 5, 6, 7, 8, 9, 10, 11, 12, 13, 14, 15, 16, 28, 31, 32, 33, 55, 70, 71, 72, or 73; and b) a pro-protein amino acid sequence of Formula VI, Formula VII, Formula VIII, Formula X, Formula XI, Formula XIV, Formula XV or SEQ ID NO. 17, 18, 19, 20, 21, 22, 23, 24, 25, 27, 29, 34, 35, 36, 37, 38, 56, 57, 58, 74, or 75. In some embodiments, the engineered yeast may be any strain as disclosed herein. In some embodiments, the engineered yeast is selected from the group comprising Kluyveromyces (e.g., K. lactis), Pichia (e.g., P. pastoris), Saccharomyces (e.g., S. cerevisiae, S. boulardii), Trichoderma (e.g., T. reesei, T. viride), and Aspergillus (e.g., A. niger).

In some embodiments, a method of inducing pancreatic secretion is provided, the method comprising administering Kluyveromyces yeast (e.g., K. lactis), genetically modified with a nucleic acid encoding a recombinant polypeptide comprising LCRF and one or both of a) a pre-protein signal peptide comprising an amino acid sequence of Formula I or SEQ ID NO. 1 and b) a pro-protein signal peptide comprising an amino acid sequence of Formula VI or SEQ ID NO. 20 or 21, thereby inducing pancreatic secretion.

In some embodiments, a method of inducing pancreatic secretion is provided, the method comprising administering Pichia yeast (e.g., P. pastoris), genetically modified with a nucleic acid encoding a recombinant polypeptide comprising LCRF and one or both of a) a pre-protein signal peptide comprising an amino acid sequence of Formula II or SEQ ID NO. 2, 3, 4, 5, 6, or 7 and b) a pro-protein signal peptide comprising an amino acid sequence of Formula VI or SEQ ID NO. 17, 20, or 21, thereby inducing pancreatic secretion.

In some embodiments, a method of inducing pancreatic secretion is provided, the method comprising administering a Saccharomyces yeast (e.g., S. cerevisiae or S. boulardii), genetically modified with a nucleic acid encoding a recombinant polypeptide comprising LCRF and one or both of a) a pre-protein signal peptide comprising an amino acid sequence of Formula III, Formula IV, Formula V, or SEQ ID NO. 8, 9, 10, 11, 12, 13, 14, 15, or 16 and b) apro-protein signal peptide comprising an amino acid sequence of Formula VI, Formula VII, Formula VIII, or SEQ ID NO. 18, 19, 20, 21, 22, 23, 24, or 25, thereby inducing pancreatic secretion.

In some embodiments, a method of inducing pancreatic secretion is provided, the method comprising administering a Trichoderma yeast (e.g., T. reesei or T. viride), genetically modified with a nucleic acid encoding a recombinant polypeptide comprising LCRF and one or both of a) a pre-protein signal peptide comprising an amino acid sequence of Formula IX or SEQ ID NO. 31, 32, or 33 and b) a pro-protein signal peptide comprising an amino acid sequence of Formula X, Formula XI, or SEQ ID NO. 34, 35, 36, 37, or 38, thereby inducing pancreatic secretion.

In some embodiments, a method of inducing pancreatic secretion is provided, the method comprising administering an Aspergillus yeast (e.g., A. niger), genetically modified with a nucleic acid encoding a recombinant polypeptide comprising LCRF and one or both of a) a pre-protein signal peptide comprising an amino acid sequence of Formula XIII or SEQ ID NO. 70, 71, 72, or 73 and b) a pro-protein signal peptide comprising an amino acid sequence of Formula XIV, Formula XV, or SEQ ID NO. 74 or 75, thereby inducing pancreatic secretion.

In some embodiments, administering may be performed via any route. In some embodiments, the route of administration is oral or topical. The therapeutically effective amount of engineered yeast may be, for example, about 100 CFUs to 10²⁰ CFUs, about 10³ to 10¹⁵ CFUs, 10⁴ to 10¹⁰ CFUs, or about 10² to about 10⁸ CFUs. In some embodiments, the therapeutically effective amount of engineered yeast is from about 100 CFUs to about 10²⁰ CFUs. In some embodiments, the therapeutically effective amount of engineered yeast is from about 10³ to about 10¹⁵ CFUs. In some embodiments, the therapeutically effective amount of engineered yeast is from about 100 CFUs, about 10³ CFUs, or about 10⁴ CFUs to about 10⁸ CFUs, about 10¹⁰ CFUs, about 10¹⁵ CFUs, or about 10²⁰ CFUs. In some embodiments, the therapeutically effective amount of engineered yeast is any amount of CFU that falls within any of the above ranges.

### Compositions of Engineered Yeast

In any method of administering the engineered yeast as a therapeutic, the engineered yeast may be incorporated into a composition suitable for oral administration to the subject. Accordingly, in some embodiments, a composition is provided, the composition comprising an engineered yeast as provided for herein. Advantageously, the engineered yeast, as disclosed herein, retain activity even after lyophilization and/or freeze-drying providing a particularly shelf-stable form for incorporating into pharmaceutical products, such as those for reconstitution prior to consumption. Accordingly, in some embodiments, the engineered yeast in the pharmaceutical composition can be provided in a lyophilized or freeze-dried form. An oral composition comprising an engineered yeast, as disclosed herein, may be in the form of a pill, tablet, capsule, microcapsule, powder, sachet, dragee, gel, liquid, suspension, solution, food product, cream or granule. In some embodiments, the composition further comprises one or more pharmaceutically acceptable excipients. In some embodiments, the pharmaceutically acceptable excipient is selected from the group including, but not limited to, carriers, solvents, co-solvents, emulsifiers, lubricants, disintegrants, binders, fillers, glidants, rheology agents, solubilizers, antimicrobials, antioxidants, preservatives, colorants, flavor agents, emollients, pH modifiers, and the like.

In some embodiments, food products may include, but are not limited to, a dairy product, a yoghurt, an ice cream, a milk-based drink, a milk-based garnish, a pudding, a milkshake, an ice tea, a fruit juice, a diet drink, a soda, a sports drink, a powdered drink mixture for dietary supplementation, an infant and baby food, a calcium-supplemented orange juice, a sauce or a soup.

Agricultural Compositions and Methods of their Use

An engineered yeast may be used to produce agricultural payload proteins such as, but not limited to, decomposition enzymes (e.g., cellulose), soil and other agricultural enzymes (e.g., lipases, proteases, polymerases, amylases, peroxidases, catalases, beta glucosidase, FDA hydrolysis, amidase, urease, phosphatase, sulfatase) fungicides (e.g., chitinase, chitin-binding proteins, cyclophilin-like proteins, defensins, lipid transfer proteins, miraculin-like proteins, nucleases, thaumatin-like proteins, and the like), insecticides (e.g., Vip1, Vip2, Vip3, Cry proteins, and the like), plant activators (e.g., branched-β-glucans, chitin oligomers, pectolytic enzymes, elicitor activity independent from enzyme activity (e.g. endoxylanase, elicitins, PaNie), avr gene products (e.g., AVR4, AVR9), viral proteins (e.g., vial coat protein, Harpins), flagellin, protein or peptide toxin (e.g., victorin), glycoproteins, glycopeptide fragments of invertase, syringolids, Nod factors (lipochitoolingo-saccharides), FACs (fatty acid amino acid conjugates), ergosterol, bacterial toxins (e.g., coronatine), and sphinganine analogue mycotoxins (e.g., fumonisin B1), which may be suitable for treating a variety of diseases and conditions. Application of one or more of the above described agricultural payload proteins to an agricultural environment, such as a crop, garden, or the like, may be beneficial for promoting soil and plant health. Accordingly, in some embodiments, a method of promoting soil and/or plant health is provided, the method comprising applying to the soil or plant an effective amount of an engineered yeast genetically modified to express a recombinant polypeptide comprising one or more of an agricultural payload protein and synthetic signal peptide. In some embodiments, the synthetic signal peptide comprises one or both of a) an pre-protein amino acid sequence of Formula II, Formula III, Formula IV, Formula V, Formula IX, Formula XIII or SEQ ID NO. 1, 2, 3, 4, 5, 6, 7, 8, 9, 10, 11, 12, 13, 14, 15, 16, 28, 31, 32, 33, 55, 70, 71, 72, or 73; and b) a pro-protein amino acid sequence of Formula VI, Formula VII, Formula VIII, Formula X, Formula XI, Formula XIV, Formula XV or SEQ ID NO. 17, 18, 19, 20, 21, 22, 23, 24, 25, 27, 29, 34, 35, 36, 37, 38, 56, 57, 58, 74, or 75. In some embodiments, the engineered yeast may be any strain as disclosed herein. In some embodiments, the engineered yeast is selected from the group comprising Kluyveromyces (e.g., K. lactis), Pichia (e.g., P. pastoris), Saccharomyces (e.g., S. cerevisiae, S. boulardii), Trichoderma (e.g., T. reesei, T. viride), and Aspergillus (e.g., A. niger).

In some embodiments, a method of promoting soil and/or plant health is provided, the method comprising administering Kluyveromyces yeast (e.g., K. lactis), genetically modified with a nucleic acid encoding a recombinant polypeptide comprising one or more of an agricultural payload protein as provided for herein and one or both of a) a pre-protein signal peptide comprising an amino acid sequence of Formula I or SEQ ID NO. 1 and b) a pro-protein signal peptide comprising an amino acid sequence of Formula VI or SEQ ID NO. 20 or 21, thereby promoting soil and/or plant health.

In some embodiments, a method of promoting soil and/or plant health is provided, the method comprising administering Pichia yeast (e.g., P. pastoris), genetically modified with a nucleic acid encoding a recombinant polypeptide comprising one or more of an agricultural payload protein as provided for herein and one or both of a) a pre-protein signal peptide comprising an amino acid sequence of Formula II or SEQ ID NO. 2, 3, 4, 5, 6, or 7 and b) a pro-protein signal peptide comprising an amino acid sequence of Formula VI or SEQ ID NO. 17, 20, or 21, thereby promoting soil and/or plant health.

In some embodiments, a method of promoting soil and/or plant health is provided, the method comprising administering a Saccharomyces yeast (e.g., S. cerevisiae or S. boulardii), genetically modified with a nucleic acid encoding a recombinant polypeptide comprising one or an agricultural payload protein as provided for herein and one or both of a) a pre-protein signal peptide comprising an amino acid sequence of Formula III, Formula IV, Formula V, or SEQ ID NO. 8, 9, 10, 11, 12, 13, 14, 15, or 16 and b) a pro-protein signal peptide comprising an amino acid sequence of Formula VI, Formula VII, Formula VIII, or SEQ ID NO. 18, 19, 20, 21, 22, 23, 24, or 25, thereby promoting soil and/or plant health.

In some embodiments, a method of promoting soil and/or plant health is provided, the method comprising administering a Trichoderma yeast (e.g., T. reesei or T. viride), genetically modified with a nucleic acid encoding a recombinant polypeptide comprising one or more of an agricultural payload protein as provided for herein and one or both of a) a pre-protein signal peptide comprising an amino acid sequence of Formula IX or SEQ ID NO. 31, 32, or 33 and b) a pro-protein signal peptide comprising an amino acid sequence of Formula X, Formula XI, or SEQ ID NO. 34, 35, 36, 37, or 38, thereby promoting soil and/or plant health.

In some embodiments, a method of promoting soil and/or plant health is provided, the method comprising administering an Aspergillus yeast (e.g., A. niger), genetically modified with a nucleic acid encoding a recombinant polypeptide comprising one or more of an agricultural payload protein as provided for herein and one or both of a) a pre-protein signal peptide comprising an amino acid sequence of Formula XIII or SEQ ID NO. 70, 71, 72, or 73 and b) a pro-protein signal peptide comprising an amino acid sequence of Formula XIV, Formula XV, or SEQ ID NO. 74 or 75, thereby promoting soil and/or plant health.

In some embodiments, administering may be performed via any route. In some embodiments, the composition is sprayed onto the soil and/or plants. The agriculturally effective amount of engineered yeast may be any amount necessary to result in the desired beneficial effect to soil and or plant health.

### ENUMERATED EMBODIMENTS

In some embodiments, the following embodiments are provided:

1. A pre-protein signal peptide comprising an amino acid sequence selected from the group consisting of Formula I, II, III, IV, V, IX, and XIII wherein Formula I is given by:

A₁-(A₂)_{w}-A₃-(A₄)ₓ-(A₅)_{y}-A₆-A₇-A₈-A₉-A₁₀-(A₁₁)_{z} (Formula I)

wherein:
w and x are each, independently, 1, 2, 3, 4, or 5;
y is 2, 3, 4, 5, 6, 7, 8, 9, 10, 11, 12, 13, 14, 15, 16, 17, 18, 19, or 20; and
z is 1, 2, or 3;
   wherein:
A₁ is methionine;
each A₂ is, independently, a neutral or positively-charged amino acid with a hydropathy index of less than about 1;
each A₃, A₅, A₈, and A₁₀ is each, independently, an amino acid with a hydropathy index greater than −1, excluding W and C;
each A₄ is, independently, a basic or neutral amino acid, excluding P, W, M, and C;
A₆ is an amino acid with a hydropathy index greater than −1, excluding W, M, and C;
A₇ is a non-aromatic amino acid with a hydropathy index of less than about 1.9 and an isoelectric point of about 5.4 to about 7.5, excluding P;
A₉ is an amino acid with a hydropathy index of greater than about −1.3; and
each A₁ is, independently, a neutral amino acid with a molecular weight of less than about 133 g/mol;
   wherein Formula II is given by:

B₁-(B₂)ᵤ-(B₃)ᵥ-(B₄)_{w}-(B₅)ₓ-(B₆)_{y}-B₇-B₈-B₉-B₁₀-(B₁₁)_{z} (Formula II)

wherein:
u and w are each, independently, 0, 1, 2, or 3;
v and z are each, independently, 1, 2, or 3;
x is 0, 1, or 2; and
y is 2, 3, 4, 5, 6, 7, 8, 9, 10, 11, 12, 13, 14, 15, 16, 17, 18, 19, or 20;
   wherein:
B₁ is methionine;
each B₂, B₄, B₆, B₈ and B₁₀ is each, independently, an amino acid with a hydropathy index of greater than about −1, excluding W and C;
each B₃ is, independently, a positively-charged or polar amino acid with a hydropathy index of less than about 1;
each B₅ is, independently, a polar amino acid with a hydropathy index of greater than about −5 and less than about −0.5, or an amino acid with an isoelectric point of about 5 to about 11, excluding P, W, M, and C;
each B₇ and B₁₁ is each, independently, a neutral amino acid with a molecular weight of less than about 133 g/mol; and
B₉ is an amino acid with a hydropathy index of greater than about −1.3;
   wherein Formula III is given by:

C₁-(C₂)ᵣ-(C₃)ₜ-(C₄)ᵤ-[(C₅)ᵥ-(C₆)_{w}]ₓ-(C₇)_{y}-(C₈)_{z}-C₉-C₁₀-C₁₁-[C₁₂-C₁₃]ₐ (Formula III)

wherein:
r is 1, 2, or 3;
t, u, y, and z are each, independently, 0, 1, 2, or 3;
v and w are each, independently, 0, 1, or 2;
a is 0 or 1; and
x is 2, 3, 4, 5, 6, 7, 8, 9, or 10;
   wherein:
C₁ is methionine;
each C₂ is, independently, an amino acid having an isoelectric point of about 5.6 to about 10.8, a molecular weight of about 105 g/mol to about 175 g/mol, a hydropathy index of about −5.1 to about 0.6, and a helicity of about 0.8 to about 1;
each C₃, C₅, C₈, and C₁₀ is each, independently, an amino acid having an isoelectric point of about 2.75 to about 10.8, a molecular weight of about 75 g/mol to about 205 g/mol, a hydropathy index of about −5.1 to about 34, and a helicity of about 0.5 to about 1.3;
each C₄ and C₇ is each, independently, an amino acid having an isoelectric point of about 5 to about 10.8, a molecular weight of about 75 g/mol to about 205 g/mol, a hydropathy index of about −5.1 to about 34, and a helicity of about 0.5 to about 1.3;
each C₆, C₉, C₁₁, and C₁₂ is each, independently, an amino acid having an isoelectric point of about 2.75 to about 9.8, a molecular weight of about 75 g/mol to about 205 g/mol; a hydropathy index of about −4 to about 34, and a helicity of about 0.5 to about 1.3; and
C₁₃ is an amino acid having an isoelectric point of about 5.6 to about 6.3, a molecular weight of about 105 g/mol to about 120 g/mol, a hydropathy index of about 0 to about 9.4, and a helicity of about 0.5 to about 1.1;
   wherein Formula IV is given by:

D₁-(D₂)_{q}-(D₃)ᵣ-(D₄)ₜ-(D₅)ᵤ-[(D₆)ᵥ-(D₇)ₓ-(D₈)_{w}-(D₉)_{y}]_{z}-D₁₀-D₁₁-D₁₂-[D₁₃-D₁₄]ₐ (Formula IV)

wherein:
q is 1, 2, or 3;
r, t, and u are each, independently, 0, 1, 2, or 3;
v, w, x, and y are each, independently, 0, 1, or 2;
a is 0 or 1; and
z is 2, 3, 4, 5, 6, 7, 8, 9, or 10;
   wherein:
D₁ is methionine;
each D₂ is, independently, an amino acid having an isoelectric point of about 2.7 to about 10.8, a molecular weight of about 75 g/mol to about 205 g/mol, a hydropathy index of about −5.1 to about 34, and a helicity of about 0.5 to about 1.3;
each D₃ is, independently, an amino acid having an isoelectric point of about 5 to about 10.8, a molecular weight of about 89 g/mol to about 205 g/mol, a hydropathy index of about −4 to about 34, and a helicity of about 0.5 to about 1.3;
each D₄, D₉ and D₁₁ is each, independently an amino acid having an isoelectric point of about 2.7 to about 10.8, a molecular weight of about 75 g/mol to about 205 g/mol, a hydropathy index of about −5.1 to about 34, and a helicity of about 0.5 to about 1.3;
each D₅ is, independently, an amino acid having an isoelectric point of about 3.2 to about 10.8, a molecular weight of about 75 g/mol to about 205 g/mol, a hydropathy index of about −5.1 to about 34, and a helicity of about 0.75 to about 1.3;
each D₆ is, independently, an amino acid having an isoelectric point from about 5 to about 10.8, a molecular weight of about 75 g/mol to about 205 g/mol, a hydropathy index of about −5.1 to about 34, and a helicity of about 0.5 to about 1.3;
each D₇ is, independently, an amino acid having an isoelectric point of about 5.4 to about 6.1, a molecular weight of about 117 g/mol to about 205 g/mol, a hydropathy index of about 2.5 to about 34, and a helicity of about 1 to about 1.3;
each D₈, D₁₀, D₁₂, and D₁₃ is each, independently, an amino acid having an isoelectric point of about 2.7 to about 10.8, a molecular weight of about 75 g/mol to about 182 g/mol, a hydropathy index of about −5.1 to about 32, and a helicity of about 0.75 to about 1.3; and
D₁₄ is an amino acid with an isoelectric point of about 2.7 to about 10.8, a molecular weight of about 75 g/mol to about 182 g/mol, a hydropathy index of about −5.1 to about 32, and a helicity of about 0.5 to about 1.3;
   wherein Formula V is given by:

E₁-[(E₂)ᵢ-(E₃)ⱼ-(E₄)_{q}]ᵣ-(E₅)ₜ-(E₆)ᵤ-(E₇)ᵥ-[(E₈)_{w}-(E₉)ₓ]_{y}-(E₁₀)_{z}-E₁₁-E₁₂-E₁₃-[E₁₄-E₁₅]ₐ (Formula V)

wherein:
i, j, q, w, x and a are each, independently, 0 or 1;
r is 1, 2, or 3;
t, u, v, and z are each, independently, 0, 1, 2, or 3; and
y is 2, 3, 4, 5, 6, 7, 8, 9, or 10;
   wherein:
E₁ is methionine;
each E₂ is, independently, an amino acid having an isoelectric point of about 3.2 to about 10.8, a molecular weight of about 105 g/mol to about 175 g/mol, a hydropathy index of about −4 to about 1, and a helicity of about 0.85 to about 1;
each E₃ is, independently, an amino acid having an isoelectric point of about 2.7 to about 10.8, a molecular weight of about 75.1 g/mol to about 205 g/mol, a hydropathy index of about −5.1 to about 33.5, and a helicity of about 0.57 to about 1.3;
each E₄ is, independently, an amino acid having an isoelectric point of about 5 to about 10.8, a molecular weight of about 105 g/mol to about 205 g/mol, a hydropathy index of about −5.1 to about 33.5, and a helicity of about 0.57 to about 1.3;
each E₅ and E₈ is each, independently, an amino acid having an isoelectric point of about 2.7 to about 10.8, a molecular weight of about 75 g/mol to about 205 g/mol, a hydropathy index of about −5.1 to about 33.5, and a helicity of about 0.57 to about 1.3;
each E₆ is, independently, an amino acid having an isoelectric point of about 5 to about 10.8, a molecular weight of about 89 g/mol to about 205 g/mol, a hydropathy index of about −5.1 to about 33.5, and a helicity of about 0.57 to about 1.3;
each E₇ is, independently, an amino acid having an isoelectric point of about 5 to about 9.75, a molecular weight of about 75 g/mol to about 205 g/mol, a hydropathy index of about −4 to about 33.5, and a helicity of about 0.79 to about 1.3;
each E₉, E₁₃, and E₁₄ is each, independently, an amino acid having an isoelectric point of about 2.7 to about 10.8, a molecular weight of about 75 g/mol to about 205 g/mol, a hydropathy index of about −5.1 to about 33.5, and a helicity of about 0.57 to about 1.3;
each E₁₀ and E₁₂ is, independently, an amino acid having an isoelectric point of about 5 to about 10.8, a molecular weight of about 75 g/mol to about 205 g/mol, a hydropathy index of about −5.1 to about 34, and a helicity of about 0.5 to about 1.3;
E₁₁ is an amino acid having an isoelectric point of about 5 to about 9.75, a molecular weight of about 89 g/mol to about 205 g/mol, a hydropathy index of about −4 to about 33.5, and a helicity of about 0.79 to about 1.3; and
E₁₅ is an amino acid having an isoelectric point of about 2.7 to about 10.8, a molecular weight of about 75 g/mol to about 205 g/mol, a hydropathy index of about −4 to about 15.5, and a helicity of about 0.57 to about 1.2;
   wherein Formula IX is given by:

F₁-(F₂)ᵥ-(F₃)_{w}-[(F₄)ₓ-(F₅)_{y}]_{z}-F₆-F₇-F₈-[F₉-F₁₀]ₐ (Formula IX)

wherein:
v and w are each, independently, 0, 1, 2, or 3;
x and y are each, independently, 0, 1, 2, 3, or 4;
a is 0 or 1; and
z is 1, 2, 3, 4, 5, 6, 7, or 8;
   wherein:
F₁ is an amino acid having an isoelectric point of about 5.4 to about 11, a molecular weight of about 89 g/mol to about 175 g/mol; a hydropathy index of about −4 to about 31, and a helicity or about 0.9 to about 1.3;
each F₂ is, independently, an amino acid having an isoelectric point of about 3 to about 11, a molecular weight of about 89 g/mol to about 205 g/mol; a hydropathy index of about −5.1 to about 34, and a helicity or about 0.5 to about 1.3;
each F₃ and F₇ is each, independently, an amino acid having an isoelectric point of about 3 to about 11, a molecular weight of about 89 g/mol to about 205 g/mol; a hydropathy index of about −5.1 to about 34, and a helicity or about 0.5 to about 1.3;
each F₄ is, independently, an amino acid having an isoelectric point of about 3 to about 11, a molecular weight of about 89 g/mol to about 205 g/mol; a hydropathy index of about −5.1 to about 34, and a helicity or about 0.5 to about 1.3;
each F₅, F₆, F₈, and F₉ is each, independently, an amino acid having an isoelectric point of about 3 to about 11, a molecular weight of about 89 g/mol to about 205 g/mol; a hydropathy index of about −5.1 to about 34, and a helicity or about 0.5 to about 1.3; and
F₁₀ is an amino acid having an isoelectric point of about 3 to about 11, a molecular weight of about 89 g/mol to about 205 g/mol; a hydropathy index of about −5.1 to about 34, and a helicity or about 0.5 to about 1.3; and
   wherein Formula XIII is given by:

L₁-(L₂)ₓ-[(L₃)ₐ-(L₄)ₐ]_{y}-[(L₅)ₐ-(L₆)ₐ-(L₇)ₐ]_{z}-(L₈)ₐ-(L₉)ₐ-(L₁₀)ₐ-(L₁₁)ₐ-(L₁₂)ₐ (Formula XIII)

wherein:
x is 1, 2, or 3;
y is 1, 2, 3, or 4;
z is 5, 6, 7, 8, 9, or 10; and
each a is, independently, 0 or 1;
   wherein:
each L₂ is, independently, an amino acid having an isoelectric point of about 2.7 to about 10.8, a molecular weight of about 75 g/mol to about 205 g/mol; a hydropathy index of about −5.1 to about 34, and a helicity or about 0.5 to about 1.3;
each L₃ and L₆ is each, independently, an amino acid having an isoelectric point of about 2.7 to about 10.8, a molecular weight of about 75 g/mol to about 205 g/mol; a hydropathy index of about −5.1 to about 34, and a helicity or about 0.5 to about 1.3;
each L₄, L₇ and L₉ is each, independently, an amino acid having an isoelectric point of about 2.7 to about 10.8, a molecular weight of about 75 g/mol to about 205 g/mol; a hydropathy index of about −5.1 to about 34, and a helicity or about 0.5 to about 1.3;
each L₅, L₈, L₁₀ and L₁₁ is each, independently, an amino acid having an isoelectric point of about 2.7 to about 10.8, a molecular weight of about 75 g/mol to about 205 g/mol; a hydropathy index of about −5.1 to about 34, and a helicity or about 0.5 to about 1.3; and
L₁₂ is an amino acid having an isoelectric point of about 2.7 to about 10.8, a molecular weight of about 75 g/mol to about 205 g/mol; a hydropathy index of about −5.1 to about 34, and a helicity or about 0.5 to about 1.3.
   2. The pre protein signal peptide of embodiment 1, wherein for Formula I:
each A₂ is, independently, an amino acid selected from the group consisting of K, R, and Q;
each A₃, A₅, A₈, and A₁₀ is each, independently, an amino acid selected from the group consisting of L, V, A, and I; and
each A₁₁ is, independently, an amino acid selected from the group consisting of A, L, and G.
   3. The pre protein signal peptide of embodiment 1, wherein for Formula II:
each B₂, B₄, B₆, B₈ and B₁₀ is each, independently, an amino acid selected from the group consisting of L, V, A, F, and I;
each B₃ is, independently, an amino acid selected from the group consisting of K, R, and Q; and
each B₇ and B₁₁ is, independently, an amino acid selected from the group consisting of A, S, G, and P.
   4. The pre protein signal peptide of embodiment 1, wherein for Formula III:
each C₂ is, independently, an amino acid selected from the group consisting of K, R, H, S, and Q;
each C₃, C₅, C₈, and C₁₀ is each, independently, an amino acid selected from the group consisting of L, V, I, A, W, Y, T, Q, S, H, C, N, D, R, P, K, G, E, and M;
each C₄ and C₇ is each, independently, an amino acid selected from the group consisting of S, N, Q, R, T, K, A, Y, H, V, I, F, G, W, C, P, and L;
each C₆, C₉, C₁₁, and C₁₂ is each, independently, an amino acid selected from the group consisting of A, S, V, G, I, L, F, C, T, K, P, Q, N, Y, E, D, M, and W; and
C₁₃ is an amino acid selected from the group consisting of P, T, and S.
   5. The pre protein signal peptide of embodiment 1, wherein for Formula IV:
each D₂ is, independently, an amino acid selected from the group consisting of K and R;
each D₃ is, independently, an amino acid selected from the group consisting of F, L, I, W, V, M, Y, P, C, A, Q, and S;
each D₄, D₉ and D₁₁ is each, independently an amino acid selected from the group consisting of L, I, F, W, V, M, Y, A, T, N, S, G, E, D, C, Q, R, H, P, and K;
each D₅ is, independently, an amino acid selected from the group consisting of S, N, Q, R, T, G, K, E, H, A, C, Y, V, W, I, F, and L;
each D₆ is, independently, an amino acid selected from the group consisting of L, I, A, T, S, G, N, R K, Y Q, C, H, W, and M;
each D₇ is, independently, an amino acid selected from the group consisting of V, W, I, L, F, and T;
each D₈, D₁₀, D₁₂, and D₁₃ is each, independently, an amino acid selected from the group consisting of A, S, T, G, V, L, C, Y, K, I, F, Q, N, H, R, E, D, and M; and
D₁₄ is an amino acid selected from the group consisting of P, Y, M, V, A, T, Q, S, N, G, I, E, D, L, F, R, K, and H.
   6. The pre protein signal peptide of embodiment 1, wherein for Formula V:
each E₂ is, independently, an amino acid selected from the group consisting of K, R, S, Q, and E;
each E₃ is, independently, an amino acid selected from the group consisting of F, L, I, W, V, Y, P, A, T, Q, N, S, G, D, R, K, and H;
each E₄ is, independently, an amino acid selected from the group consisting of K, R, H, S, C, P, Y, M, V, W, I, L, and F;
each E₅ and E₈ is each, independently, an amino acid selected from the group consisting of L, I, F, V, C, A, Y, T, Q, N, S, K, H, W, G, D, M, P, E, and R;
each E₆ is, independently, an amino acid selected from the group consisting of T, Q, S, A, C, R, K, H, P, V, W, I, F, and L;
each E₇ is, independently, an amino acid selected from the group consisting of S, G, K, A, C, Y, V, and W;
each E₉, E₁₃, and E₁₄ is each, independently, an amino acid selected from the group consisting of A, T, G, S, V, I, L, Y, W, F, C, Q, N, P, E, M, R, K, D, and H;
each E₁₀ and E₁₂ is each, independently, an amino acid selected from the group consisting of L, F, I, V, C, Y, T, Q, N, S, K, H, M, G, A, W, D, P, E, and R.
E₁₁ is an amino acid selected from the group consisting of V, W, I, C, L, A, T, S, and K; and
E₁₅ is an amino acid selected from the group consisting of S, N, R, T, G, K, E, D, P, and Y.
   7. The pre-protein signal peptide of embodiment 1 wherein for Formula IX:
F₁ is an amino acid selected from the group consisting of M, F, L, A, S, or R;
each F₂ is, independently, an amino acid selected from the group consisting of K, R, H, S, G, N, Q, E, T, A, C, P, Y, V, W, I, L, or F;
each F₃ and F₇ is each, independently, an amino acid selected from the group consisting of S, Q, R, T, K, H, I, F, L, P, N, G, E, D, A, Y, M, V, W, or C;
each F₄ is, independently, an amino acid selected from the group consisting of L, I, V, M, A, F, W, Y, P, C, T, Q, N, S, G, E, R, K, or H;
each F₅, F₆, F₈, and F₉ is each, independently, an amino acid selected from the group consisting of A, C, G, S, V, L, T, F, Q, N, P, Y, E, K, H, W, I, M, R, or D; and
F₁₀ is an amino acid selected from the group consisting of P, C, Y, M, V, A, T, Q, S, N, W, G, I, E, D, L, F, R, K, or H.
   8. The pre-protein signal peptide of embodiment 1 wherein for Formula XIII:
each L₂ is, independently, an amino acid selected from the group consisting of R, K, H, S, G, N, Q, D, T, A, C, P, Y, M, V, W, I, F, and L;
each L₃ and L₆ is each, independently, an amino acid selected from the group consisting of S, N, Q, R, T, K, P, G, E, H, D, A, C, Y, M, V, W, I, F, and L;
each L₄, L₇ and L₉ is each, independently, an amino acid selected from the group consisting of L, F, I, W, V, T, M, Y, P, C, A, Q, N, S, G, E, D, R, K, and H;
each L₅, L₈, L₁₀ and L₁₁ is each, independently, an amino acid selected from the group consisting of A, T, G, S, C, P, I, L, F, R, V, Q, Y, K, N, E, D, H, M, and W; and
L₁₂ is an amino acid selected from the group consisting of P, T, S, D, C, Y, M, V, A, Q, N, W, G, I, E, L, F, R, K, and H.
   9. The pre-protein signal peptide of embodiment 1, wherein the signal peptide comprises an amino acid sequence having at least 90%, 91%, 92%, 93%, 94%, 95%, 96%, 97%, 98%, or 99% identity to an amino acid sequence selected from the group consisting of SEQ ID NO. 1, 2, 3, 4, 5, 6, 7, 8, 9, 10, 11, 12, 13, 14, 15, 16, 28, 31, 32, 33, 55, 70, 71, 72, or 73.
   10. The pre-protein signal peptide of embodiment 1, wherein the amino acid sequence is selected from the group consisting of SEQ ID NO. 1, 2, 3, 4, 5, 6, 7, 8, 9, 10, 11, 12, 13, 14, 15, 16, 28, 31, 32, 33, 55, 70, 71, 72, or 73.
   11. A pre-protein signal peptide comprising an amino acid sequence having at least 90%, 91%, 92%, 93%, 94%, 95%, 96%, 97%, 98%, 99% or 100% identity to SEQ ID NO. 1, 2, 3, 4, 5, 6, 7, 8, 9, 10, 11, 12, 13, 14, 15, 16, 28, 31, 32, 33, 55, 70, 71, 72, or 73.
   12. A pro-protein signal peptide comprising an amino acid sequence selected from the group consisting of Formula VI, VII, VIII, X, XI, XIV, and XV;
   wherein Formula VI is given by:

G₁-G₂-G₃-G₄-G₅-G₆-G₇-G₈-G₉-G₁₀-G₁₁-G₁₂-G₁₃-G₁₄-G₁₅-G₁₆-G₁₇-G₁₈-G₁₉-G₂₀-G₂₁-G₂₂-G₂₃-G₂₄-G₂₅ (Formula VI)

wherein:
G₁ is an amino acid selected from the group consisting of I, L, F, V, A, N, S, D, R, and K;
G₂ is an amino acid selected from the group consisting of P, S, N, G, and E;
G₃ is an amino acid selected from the group consisting of L, F, I, V, Y, A, S, R, and H;
G₄ is an amino acid selected from the group consisting of V, M, P, Y, A, T, S, N, K, and H;
G₅ is an amino acid selected from the group consisting of A, G, R, Y, K, D, M, V, W, I, and L;
G₆ is an amino acid selected from the group consisting of N, R, and K;
G₇ is an amino acid selected from the group consisting of V, P, A, T, Q, G, E, D, R, and K;
G₈ is an amino acid selected from the group consisting of P, Y, T, Q, S, N, W, F, R, K, and H;
G₉ is an amino acid selected from the group consisting of F, L, A, Q, N, S, E, G, D, and H;
G₁₀ is an amino acid selected from the group consisting of H, S, N, D, Q, E, T, Y, M, V, I, and L;
G₁₁ is an amino acid selected from the group consisting of S, R, T, G, K, E, D, and P;
G₁₂ is an amino acid selected from the group consisting of D, E, Q, N, A, and V;
G₁₃ is an amino acid selected from the group consisting of N, S, E, D, T, H, K, A, and P;
G₁₄ is an amino acid selected from the group consisting of G, S, N, H, E, C, Y, L, and F;
G₁₅ is an amino acid selected from the group consisting of S, T, and H;
G₁₆ is an amino acid selected from the group consisting of E, D, Q, N, S, T, K, and A;
G₁₇ is an amino acid selected from the group consisting of W, N, D, and R;
G₁₈ is an amino acid selected from the group consisting of L and F;
G₁₉ is an amino acid selected from the group consisting of Y, V, A, Q, N, S, E, D, L, R, K, and H;
G₂₀ is an amino acid selected from the group consisting of K, R, S, and I;
G₂₁ is R;
G₂₂ is an amino acid selected from the group consisting of D, E, N, S, T, G, A, Y, and L;
G₂₃ and G₂₄ are each, independently, an amino acid selected from the group consisting of V, P, Y, I, A, E, K, F, T, S, G, D, M, and N; and
G₂₅ is an amino acid selected from the group consisting of Y, P, A, T, Q, S, E, F, and H;
   wherein Formula VII is given by:

(H₁)ₘ-(H₂)ₘ-(H₃)ₘ-(H₄)ₘ-(H₅)ₘ-(H₆)ₘ-(H₇)ₘ-(H₈)ₘ-(H₉)ₘ-(H₁₀)ₘ-(H₁₁)ₘ- (H₁₂)ₘ-(H₁₃)ₘ-(H₁₄)ₘ-(H₁₅)ₘ-(H₁₆)ₘ-(H₁₇)ₘ-(H₁₈)ₘ-(H₁₉)ₘ-(H₂₀)ₘ-(H₂₁)ₘ-(H₂₂)ₘ-(H₂₃)ₘ-(H₂₄)ₘ-(H₂₅)ₘ-(H₂₆)ₘ-(H₂₇)ₘ-(H₂₈)ₘ-(H₂₉)ₘ-(H₃₀)ₘ-(H₃₁)ₘ-(H₃₂)ₘ-(H₃₃)ₘ-(H₃₄)ₘ-(H₃₅)ₘ-(H₃₆)ₘ-H₃₇-H₃₈-H₃₉-H₄₀ (Formula VII)

wherein:
each m is, independently, 0, 1, or 2;
   wherein:
each H₁ is, independently, an amino acid selected from the group consisting of E, D, S, L, G, Q, and A;
each H₂ and H₂₈ is each, independently, an amino acid selected from the group consisting of P, S, R, T, N, G, D, K, and A;
each H₃ is, independently, an amino acid selected from the group consisting of W and Y;
each H₄ is, independently, an amino acid selected from the group consisting of S, N, A, P, and V;
each H₅ and H₃₀ is each, independently, an amino acid selected from the group consisting of T, Q, A, E, F, and S;
each H₆ is, independently, an amino acid selected from the group consisting of L, F, and I;
each H₇ is, independently, an amino acid selected from the group consisting of F, V, M, T, S, and K;
each H₈ is, independently, an amino acid selected from the group consisting of V, P, I, A, S, and K;
each H₉ and H₁₇ is each, independently, an amino acid selected from the group consisting of T, G, V, W, and A;
each H₁₀ is, independently, an amino acid selected from the group consisting of R, H, S, G, N, E, T, and V;
each H₁₁ is, independently, an amino acid selected from the group consisting of S, G, D, A, and M;
each H₁₂ is, independently, an amino acid selected from the group consisting of T, S, E, G, D, K, and H;
each H₁₃ is, independently, an amino acid selected from the group consisting of L, M, Y, N, S, D, and K;
each H₁₄ is, independently, an amino acid selected from the group consisting of D, Q, N, S, K, and C;
each H₁₅ is, independently, an amino acid selected from the group consisting of E, S, D, L, and G;
each H₁₆ is, independently, an amino acid selected from the group consisting of I, L, V, M, A, and T;
each H₁₈ is, independently, an amino acid selected from the group consisting of D, E, S, T, K, and G;
each H₁₉ is, independently, an amino acid selected from the group consisting of Y, F, and L;
each H₂₀ is, independently, an amino acid selected from the group consisting of N, Q, S, T, R, and F;
each H₂₁ and H₃₄ is each, independently, an amino acid selected from the group consisting of S, K, T, A, Y, M, and F;
each H₂₂ is, independently, an amino acid selected from the group consisting of T, Q, S, D, C, V, and L;
each H₂₃ is, independently, an amino acid selected from the group consisting of G, S, K, N, H, D, W, and L;
each H₂₄ is, independently, an amino acid selected from the group consisting of I, L, V, P, N, and E;
each H₂₅ and H₃₃ is each, independently, an amino acid selected from the group consisting of A, T, G, R, Y, L, F, and E;
each H₂₆ and H₄₀ is each, independently, an amino acid selected from the group consisting of V, I, F, M, L, A, and T;
each H₂₇ is, independently, an amino acid selected from the group consisting of D, E, Q, N, S, A, and I;
each H₂₉ is, independently, an amino acid selected from the group consisting of E, D, T, A, Y, M, V, I, F, and L;
each H₃₁ is, independently, an amino acid selected from the group consisting of F, W, V, M, S, G, and R;
each H₃₂ is, independently, an amino acid selected from the group consisting of H, S, E, G, and T;
each H₃₅ is, independently, an amino acid selected from the group consisting of R, K, S, and Q;
each H₃₆ is, independently, an amino acid selected from the group consisting of H, R, S, T, A, V, W, and L;
H₃₇ is an amino acid selected from the group consisting of K, Q, D, A, and I;
H₃₈ is an amino acid selected from the group consisting of R, K, T, and F; and
H₃₉ is an amino acid selected from the group consisting of D, N, S, T, K, A, Y, and L;
   wherein Formula VIII is given by:

(I₁)ₘ-(I₂)ₘ-(I₃)ₘ-(I₄)ₘ-(I₅)ₘ-(I₆)ₘ-(I₇)ₓ-(I₈)ₘ-(I₉)ₘ-(I₁₀)ₘ-(I₁₁)ₓ- (I₁₂)ₘ-(I₁₃)ₓ-(I₁₄)ₓ-(I₁₅)ₘ-(I₁₆)ₓ-(I₁₇)ₘ-I₁₈-I₁₉-I₂₀-I₂₁-I₂₂-I₂₃ (Formula VIII)

wherein:
each m is, independently, 0, 1, or 2; and
each x is, independently, 0, 1, 2, 3, or 4;
   wherein:
each I₁ and I₆ is each, independently, an amino acid selected from the group consisting of S, Q, E, A, I, G, V, R, T, and Y;
each I₂ is, independently, an amino acid selected from the group consisting of T, S, E, R, P, V, I, and F;
each I₃ is, independently, L.
each I₄ is, independently, an amino acid selected from the group consisting of T, N, K, and M;
each I₅ is, independently, an amino acid selected from the group consisting of P, A, and D;
each I₇ is, independently, an amino acid selected from the group consisting of T, S, K, H, Y, V, and F;
each I₈ and I₁₅ is each, independently, an amino acid selected from the group consisting of F, L, W, A, T, M, Y, and C;
each I₉ is, independently, an amino acid selected from the group consisting of I, L, and V;
each I₁₀ and I₁₆ is each, independently, an amino acid selected from the group consisting of G, S, N, E, D, A, K, H, C, P, and F;
each I₁₁ is, independently, an amino acid selected from the group consisting of I, L, V, A, T, and S;
each I₁₂ is, independently, an amino acid selected from the group consisting of T, N, A, E, and G;
each I₁₃ is, independently, an amino acid selected from the group consisting of E, Q, S, T, R, K, A, L, D, and F;
each I₁₄ is, independently, an amino acid selected from the group consisting of T, S, Q, F, A, G, V, I, and L;
each I₁₇ is, independently, an amino acid selected from the group consisting of I, L, V, N, A, T, and S;
I₁₈ and I₂₁ are each, independently, an amino acid selected from the group consisting of R, K, Q, and A;
I₁₉ is an amino acid selected from the group consisting of H, R, S, N, T, A, V, and W;
I₂₀ is an amino acid selected from the group consisting of K, N, Q, D, E, A, and I;
I₂₂ is an amino acid selected from the group consisting of D, N, S, A, Y, and L; and
I₂₃ is an amino acid selected from the group consisting of V, I, L, F, and A;
   wherein Formula X is given by:

(J₁)_{z}-(J₂)_{z}-(J₃)_{z}-(J₄)_{z}-(J₅)_{z}-(J₆)_{z}-(J₇)_{z}-(J₈)_{z}-(J₉)_{z}-(J₁₀)_{z}-(J₁₁)_{z}- (J₁₂)_{z}-(J₁₃)_{z}-(J₁₄)_{z}-(J₁₅)_{z}-(J₁₆)_{z}-(J₁₇)_{z}-(J₁₈)_{z}-(J₁₉)_{z}-(J₂₀)_{z}-(J₂₁)_{z}-J₂₂-J₂₃-J₂₄-J₂₅ (Formula X)

wherein:
each z is, independently, 0, 1, 2, 3, 4, or 5;
   wherein:
each J₁ is, independently, an amino acid selected from the group consisting of H, K, G, A, P, F, and L;
each J₂ is, independently, an amino acid selected from the group consisting of D, E, N, G, P, H, T, R, K, and A;
each J₃ is, independently, an amino acid selected from the group consisting of G, A, P, V, and L;
each J₄ is, independently, an amino acid selected from the group consisting of F, I, P, A, S, E, D, R, and K;
each J₅ is, independently, an amino acid selected from the group consisting of S, R, T, G, K, E, D, and C;
each J₆ is, independently, an amino acid selected from the group consisting of T, S, A, D, and F;
each J₇ is, independently, an amino acid selected from the group consisting of D, E, N, G, P, H, T, R, K, and A;
each J₈ is, independently, an amino acid selected from the group consisting of Y, C, A, W, I, S, E, D, F, L, R, and K;
each J₉ is, independently, an amino acid selected from the group consisting of H, K, N, D, G, T, A, C, Y, V, and L;
each J₁₀ is, independently, an amino acid selected from the group consisting of L, V, A, G, E, I, P, and R;
each J₁₁ is, independently, an amino acid selected from the group consisting of I, W, V, Y, P, T, N, S, R, and K;
each J₁₂ is, independently, an amino acid selected from the group consisting of A, G, Q, N, R, Y, E, D, and L;
each J₁₃ is, independently, an amino acid selected from the group consisting of I, L, W, V, M, Y, P, A, S, and G;
each J₁₄ is, independently, an amino acid selected from the group consisting of V, C, L, F, A, T, N, G, and R;
each J₁₅ is, independently, an amino acid selected from the group consisting of G, S, R, K, A, T, H, E, W, L, and F;
each J₁₆ is, independently, an amino acid selected from the group consisting of D, E, Q, S, H, T, R, G, Y, V, F, and L;
each J₁₇ is, independently, an amino acid selected from the group consisting of E, S, G, Y, I, and L;
each J₁₈ is, independently, an amino acid selected from the group consisting of A, S, P, H, and V;
each J₁₉ is, independently, an amino acid selected from the group consisting of N, E, R, K, and A;
each J₂₀ is, independently, an amino acid selected from the group consisting of R, T, V, I, and L;
each J₂₁ is, independently, an amino acid selected from the group consisting of L, V, A, G, E, I, P, and R;
J₂₂ is an amino acid selected from the group consisting of K, R, D, T, M, and W;
J₂₃ is an amino acid selected from the group consisting of R, T, V, I, and L;
J₂₄ is an amino acid selected from the group consisting of S, N, G, E, D, P, and W; and
J₂₅ is an amino acid selected from the group consisting of A, T, S, Y, M, V, and L;
   wherein Formula XI is given by:

(K₁)_{b}-(K₂)_{b}-(K₃)_{b}-(K₄)_{b}-(K₅)_{b}-(K₆)_{b}-(K₇)_{b}-(K₈)_{b}-(K₉)_{b}-(K₁₀)_{b}-(K₁₁)_{b}- (K₁₂)_{b}-(K₁₃)_{b}-(K₁₄)_{b}-(K₁₅)_{b}-(K₁₆)_{b}-(K₁₇)_{b}-(K₁₈)_{b}-(K₁₉)_{b}-(K₂₀)_{b}-(K₂₁)_{b}-(K₂₂)_{b}-(K₂₃)_{b}-(K₂₄)_{b}-(K₂₅)_{b}-(K₂₆)_{b}-(K₂₇)_{b}-(K₂₈)_{b}-(K₂₉)_{b}-(K₃₀)_{b}-(K₃₁)_{b}-(K₃₂)_{b}-(K₃₃)_{b}-(K₃₄)_{b}-(K₃₅)_{b}-(K₃₆)_{b}-(K₃₇)_{b}-(K₃₈)_{b}-(K₃₉)_{b}-(K₄₀)_{b}-(K₄₁)_{b}-(K₄₂)_{b}-(K₄₃)_{b}-(K₄₄)_{b}-(K₄₅)_{b}-(K₄₆)_{b}-(K₄₇)_{b}-(K₄₈)_{b}-(K₄₉)_{b}-(K₅₀)_{b}-(K₅₁)_{b}-(K₅₂)_{b}-(K₅₃)_{b}-(K₅₄)_{b}-(K₅₅)_{b}-(K₅₆)_{b}-(K₅₇)_{b}-(K₅₈)_{b}-(K₅₉)_{b}-(K₆₀)_{b}-(K₆₁)_{b}-(K₆₂)_{b}-(K₆₃)_{b}-(K₆₄)_{b}-(K₆₅)_{b}-(K₆₆)_{b}-(K₆₇)_{b}-(K₆₈)_{b}-(K₆₉)_{b}-(K₇₀)_{b}-(K₇₁)_{b}-(K₇₂)_{b}-(K₇₃)_{b}-(K₇₄)_{b}-(K₇₅)_{b}-(K₇₆)_{b}-(K₇₇)_{b}-(K₇₈)_{b}-(K₇₉)_{b}-(K₈₀)_{b}-(K₈₁)_{b}-(K₈₂)_{b}-(K₈₃)_{b}-(K₈₄)_{b}-(K₈₅)_{b}-(K₈₆)_{b}-(K₈₇)_{b}-(K₈₈)_{b}-K₈₉-K₈₉-K₈₉-K₈₉-K₈₉ (Formula XI)

wherein:
each b is, independently, 0, 1, 2, or 3;
   wherein:
each K₁ is, independently, an amino acid selected from the group consisting of S, G, D, A, C, P, and Y;
each K₂ is, independently, an amino acid selected from the group consisting of Q, S, E, T, R, K, G, A, Y, M, V, and I;
each K₃ is, independently, an amino acid selected from the group consisting of G, S, N, T, Q, D, P, L, F, V, K, A, and C;
each K₄ is, independently, an amino acid selected from the group consisting of R, G, N, D, A, P, Y, and L;
each K₅ is, independently, an amino acid selected from the group consisting of E, A, V, Q, G, Y, M, I, and L;
each K₆ is, independently, an amino acid selected from the group consisting of S, Q, R, T, D, G, E, A, and K;
each K₇ is, independently, an amino acid selected from the group consisting of N, Q, R, H, K, A, I, F, and L;
each K₈ is, independently, an amino acid selected from the group consisting of A, T, Q, G, R, K, D, L, F, C, V, S, and H;
each K₉ is, independently, an amino acid selected from the group consisting of G, S, N, T, Q, D, P, L, F, V, K, A, and C;
each K₁₀ is, independently, an amino acid selected from the group consisting of K, H, E, A, Y, L, and F;
each K₁₁ is, independently, an amino acid selected from the group consisting of S, T, K, E, A, C, W, F, and L;
each K₁₂ is, independently, an amino acid selected from the group consisting of K, R, H, S, Q, D, E, and A;
each K₁₃ is, independently, an amino acid selected from the group consisting of G, S, T, E, P, W, R, N, and Q;
each K₁₄ is, independently, an amino acid selected from the group consisting of D, Q, S, G, V, E, N, H, R, P, and F;
each K₁₅ is, independently, an amino acid selected from the group consisting of C, A, M, V, S, E, G, I, F, and L;
each K₁₆ is, independently, an amino acid selected from the group consisting of R, K, S, Q, T, Y, N, V, I, L, and C;
each K₁₇ is, independently, an amino acid selected from the group consisting of A, G, S, Q, Y, E, D, H, and I;
each K₁₈ is, independently, an amino acid selected from the group consisting of R, K, S, Q, T, Y, N, V, I, L, and C;
each K₁₉ is, independently, an amino acid selected from the group consisting of E, D, T, H, K, G, P, V, and L;
each K₂₀ is, independently, an amino acid selected from the group consisting of F, L, I, V, M, T, G, and R;
each K₂₁ is, independently, an amino acid selected from the group consisting of E, D, S, G, A, C, and P;
each K₂₂ is, independently, an amino acid selected from the group consisting of D, T, G, A, Y, N, S, C, P, W, and I;
each K₂₃ is, independently, an amino acid selected from the group consisting of G, S, N, E, D, Y, and L;
each K₂₄ is, independently, an amino acid selected from the group consisting of T, S, E, G, P, and I;
each K₂₅ is, independently, an amino acid selected from the group consisting of K, S, G, T, and L;
each K₂₆ is, independently, an amino acid selected from the group consisting of S, G, K, E, D, P, and F;
each K₂₇ is, independently, an amino acid selected from the group consisting of P, A, E, L, T, Q, S, G, K, Y, F, C, V, W, and R;
each K₂₈ is, independently, an amino acid selected from the group consisting of E, D, Q, S, T, P, and L;
each K₂₉ is, independently, an amino acid selected from the group consisting of A, T, S, E, V, W, and I;
each K₃₀ is, independently, an amino acid selected from the group consisting of K, H, S, G, N, Q, P, and Y;
each K₃₁ is, independently, an amino acid selected from the group consisting of L, F, V, P, A, N, G, and H;
each K₃₂ is, independently, an amino acid selected from the group consisting of A, G, N, P, R, E, and K;
each K₃₃ is, independently, an amino acid selected from the group consisting of R, S, N, A, P, Y, V, I, F, and G;
each K₃₄ is, independently, an amino acid selected from the group consisting of E, S, T, V, I, H, A, P, F, and L;
each K₃₅ is, independently, an amino acid selected from the group consisting of A, T, Q, P, R, V, N, E, and L;
each K₃₆ is, independently, an amino acid selected from the group consisting of R, K, H, G, Q, D, T, Y, and F;
each K₃₇ is, independently, an amino acid selected from the group consisting of D, E, N, T, C, Y, V, I, and L;
each K₃₈ is, independently, an amino acid selected from the group consisting of S, Q, R, T, D, G, E, A, and K;
each K₃₉ is, independently, an amino acid selected from the group consisting of K, S, G, Q, D, E, A, M, I, and L;
each K₄₀ is, independently, an amino acid selected from the group consisting of H, K, S, D, E, T, P, and L;
each K₄₁ is, independently, an amino acid selected from the group consisting of A, T, S, N, P, V, L, and F;
each K₄₂ is, independently, an amino acid selected from the group consisting of K, D, M, V, I, L, and F;
each K₄₃ is, independently, an amino acid selected from the group consisting of G, S, N, T, Q, D, P, L, F, V, K, A, and C;
each K₄₄ is, independently, an amino acid selected from the group consisting of L, T, F, V, P, A, K, and I;
each K₄₅ is, independently, an amino acid selected from the group consisting of G, S, K, N, T, Q, D, A, P, L, F, and V;
each K₄₆ is, independently, an amino acid selected from the group consisting of L, F, Q, S, G, and D;
each K₄₇ is, independently, an amino acid selected from the group consisting of S, R, E, A, P, V, W, and L;
each K₄₈ is, independently, an amino acid selected from the group consisting of A, S, V, G, Q, R, E, D, L, T, K, F, C, and H;
each K₄₉ is, independently, an amino acid selected from the group consisting of E, S, T, R, G, A, P, and L;
each K₅₀ is, independently, an amino acid selected from the group consisting of S, N, R, A, P, and Y;
each K₅₁ is, independently, an amino acid selected from the group consisting of G, A, T, H, M, V, L, and F;
each K₅₂ is, independently, an amino acid selected from the group consisting of S, T, H, A, C, M, and L;
each K₅₃ is, independently, an amino acid selected from the group consisting of G, S, T, E, P, W, R, N, and Q;
each K₅₄ is, independently, an amino acid selected from the group consisting of S, H, Y, F, N, Q, R, T, G, and K;
each K₅₅ is, independently, an amino acid selected from the group consisting of A, T, Q, E, M, V, I, L, and F;
each K₅₆ is, independently, an amino acid selected from the group consisting of S, N, E, A, P, F, and L;
each K₅₇ is, independently, an amino acid selected from the group consisting of D, S, R, K, A, V, W, I, and F;
each K₅₈ is, independently, an amino acid selected from the group consisting of K, S, G, D, T, L, R, E, Y, and N;
each K₅₉ is, independently, an amino acid selected from the group consisting of S, R, G, A, V, and F;
each K₆₀ is, independently, an amino acid selected from the group consisting of A, T, Q, G, R, K, D, L, F, C, V, S, and H;
each K₆₁ is, independently, an amino acid selected from the group consisting of R, S, G, N, E, T, A, and V;
each K₆₂ is, independently, an amino acid selected from the group consisting of E, S, T, V, I, H, A, P, F, and L;
each K₆₃ is, independently, an amino acid selected from the group consisting of A, G, S, Q, R, E, D, V, L, T, K, F, C, and H;
each K₆₄ is, independently, an amino acid selected from the group consisting of E, A, V, Q, G, Y, M, I, and L;
each K₆₅ is, independently, an amino acid selected from the group consisting of G, S, T, E, P, W, R, N, and Q;
each K₆₆ is, independently, an amino acid selected from the group consisting of A, G, P, M, N, V, and S;
each K₆₇ is, independently, an amino acid selected from the group consisting of T, Q, E, N, S, A, Y, V, W, and F;
each K₆₈ is, independently, an amino acid selected from the group consisting of I, V, P, and A;
each K₆₉ is, independently, an amino acid selected from the group consisting of D, Q, S, G, V, E, N, H, R, P, and F;
each K₇₀ is, independently, an amino acid selected from the group consisting of G, S, R, N, T, Y, L, and F;
each K₇₁ is, independently, an amino acid selected from the group consisting of E, D, N, S, T, H, and Y;
each K₇₂ is, independently, an amino acid selected from the group consisting of L, I, W, V, A, T, S, E, R, and K;
each K₇₃ is, independently, an amino acid selected from the group consisting of G, S, K, A, C, F, N, T, Q, D, P, L, and V;
each K₇₄ is, independently, an amino acid selected from the group consisting of A, S, N, P, K, V, I, and L;
each K₇₅ is, independently, an amino acid selected from the group consisting of P, A, E, L, T, Q, S, G, K, Y, F, C, V, W, and R;
each K₇₆ is, independently, an amino acid selected from the group consisting of L, T, F, V, P, A, K, and I;
each K₇₇ is, independently, an amino acid selected from the group consisting of M, V, Y, L, A, N, E, and H;
each K₇₈ is, independently, an amino acid selected from the group consisting of D, T, G, A, Y, N, S, C, P, W, and I;
each K₇₉ is, independently, an amino acid selected from the group consisting of A, S, V, G, Q, R, E, D, L, T, K, F, C, and H;
each K₈₀ is, independently, an amino acid selected from the group consisting of K, R, S, A, P, V, I, and L;
each K₈₁ is, independently, an amino acid selected from the group consisting of F, L, V, A, T, S, E, D, R, and K;
each K₈₂ is, independently, an amino acid selected from the group consisting of L, F, M, A, N, G, and E;
each K₈₃ is, independently, an amino acid selected from the group consisting of D, S, H, A, V, I, F, and L;
each K₈₄ is, independently, an amino acid selected from the group consisting of A, T, Q, S, R, V, L, G, H, F, K, D, and C;
each K₈₅ is, independently, an amino acid selected from the group consisting of T, Q, E, N, S, A, Y, V, W, and F;
each K₈₆ is, independently, an amino acid selected from the group consisting of A, P, R, Y, K, D, M, L, and F;
each K₈₇ is, independently, an amino acid selected from the group consisting of N, S, D, T, A, P, and L;
each K₅₅ is, independently, an amino acid selected from the group consisting of R, S, N, A, P, Y, V, I, F, and G;
K₈₉ is an amino acid selected from the group consisting of K, R, H, G, E, T, Y, and I;
K₉₀ is an amino acid selected from the group consisting of R, S, G, N, Q, A, Y, and W;
K₉₁ is an amino acid selected from the group consisting of V, I, and F;
K₉₂ is an amino acid selected from the group consisting of A, G, P, M, N, V, and S; and
K₉₃ is an amino acid selected from the group consisting of E, D, Q, S, R, K, M, and L;
   wherein Formula XIV is given by:

(M₁)_{b}-(M₂)_{b}-(M₃)_{b}-(M₄)_{b}-(M₅)_{b}-(M₆)_{b}-(M₇)_{b}-(M₈)_{b}-(M₉)_{b}-(M₁₀)_{b}-(M₁₁)_{b}- (M₁₂)_{b}-(M₁₃)_{b}-(M₁₄)_{b}-(M₁₅)_{b}-(M₁₆)_{b}-(M₁₇)_{b}-(M₁₈)_{b}-(M₁₉)_{b}-(M₂₀)_{b}-(M₂₁)_{b}-(M₂₂)_{b}-(M₂₃)_{b}-(M₂₄)_{b}-(M₂₅)_{b}-(M₂₆)_{b}-(M₂₇)_{b}-(M₂₈)_{b}-(M₂₉)_{b}-(M₃₀)_{b}-(M₃₁)_{b}-(M₃₂)_{b}-(M₃₃)_{b}-(M₃₄)_{b}-(M₃₅)_{b}-(M₃₆)_{b}-(M₃₇)_{b}-(M₃₈)_{b}-(M₃₉)_{b}-(M₄₀)_{b}-(M₄₁)_{b}-(M₄₂)_{b}-(M₄₃)_{b}-(M₄₄)_{b}-(M₄₅)_{b}-(M₄₆)_{b}-(M₄₇)_{b}-(M₄₈)_{b}-(M₄₉)_{b}-(M₅₀)_{b}-(M₅₁)_{b}-(M₅₂)_{b}-(M₅₃)_{b}-(M₅₄)_{b}-(M₅₅)_{b}-(M₅₆)_{b}-(M₅₇)_{b}-(M₅₈)_{b}-(M₅₉)_{b}-(M₆₀)_{b}-(M₆₁)_{b}-(M₆₂)_{b}-(M₆₃)_{b}-(M₆₄)_{b}-(M₆₅)_{b}-(M₆₆)_{b}-(M₆₇)_{c}-(M₆₈)_{c}-(M₆₉)_{c}-(M₇₀)_{c} (Formula XIV);

wherein:
each b is, independently, 0, 1, 2, or 3; and
each c is, independently, 1 or 2;
   wherein:
each M₁ is, independently, an amino acid selected from the group consisting of A, T, C, S, Y, E, H, V, W, I, L, F, G, Q, N, P, R, K, D, and M;
each M₂ is, independently, an amino acid selected from the group consisting of S, T, A, N, R, G, E, P, V, F, L, Q, K, H, D, I, C, Y, M, and W;
each M₃ is, independently, an amino acid selected from the group consisting of G, S, R, A, T, Q, E, D, C, Y, V, I, L, and N;
each M₄ is, independently, an amino acid selected from the group consisting of R, H, N, Q, E, A, Y, M, V, W, F, and L;
each M₅ is, independently, an amino acid selected from the group consisting of P, Y, A, T, Q, S, G, D, R, K, C, V, I, L, and H;
each M₆ is, independently, an amino acid selected from the group consisting of T, Q, N, S, A, E, G, D, H, P, F, L, C, K, V, R, Y, I, M, and W;
each M₇ is, independently, an amino acid selected from the group consisting of A, G, S, Q, N, K, D, T, C, Y, E, H, V, W, I, L, F, P, R, and M;
each M₈ is, independently, an amino acid selected from the group consisting of T, Q, N, S, A, G, C, R, K, P, Y, M, V, I, L, F, E, W, D, and H;
each M₉ is, independently, an amino acid selected from the group consisting of G, S, H, P, R, A, T, Q, E, D, C, Y, V, I, L, N, W, F, K, and M;
each M₁₀ is, independently, an amino acid selected from the group consisting of Q, E, and W;
each M₁₁ is, independently, an amino acid selected from the group consisting of V, I, L, F, C, A, and T;
each M₁₂ is, independently, an amino acid selected from the group consisting of S, G, A, N, Q, R, T, K, E, H, D, P, I, F, V, C, Y, L, M, and W;
each M₁₃ is, independently, an amino acid selected from the group consisting of T, Q, N, S, D, P, F, A, E, G, H, L, C, K, V, R, Y, I, M, and W;
each M₁₄ is, independently, an amino acid selected from the group consisting of L, F, I, V, M, Y, A, T, Q, N, S, D, K, P, E, R, H, G, and C;
each M₁₅ is, independently, an amino acid selected from the group consisting of S, P, V, E, T, A, F, L, N, R, G, Q, K, H, D, I, C, Y, M, and W;
each M₁₆ is, independently, an amino acid selected from the group consisting of T, S, A, E, G, C, R, P, Y, M, V, W, I, F, L, Q, N, D, H, and K;
each M₁₇ is, independently, an amino acid selected from the group consisting of D, E, Q, T, K, P, F, N, S, G, A, Y, R, and V;
each M₁₈ is, independently, an amino acid selected from the group consisting of G, S, H, P, R, D, N, A, T, Q, E, C, Y, V, I, L, W, F, K, and M;
each M₁₉ is, independently, an amino acid selected from the group consisting of T, P, F, S, A, E, G, C, R, Y, M, V, W, I, L, Q, N, D, H, and K;
each M₂₀ is, independently, an amino acid selected from the group consisting of L, F, I, V, Y, A, T, Q, S, D, M, N, K, P, E, R, H, G, and C;
each M₂₁ is, independently, an amino acid selected from the group consisting of F, L, W, Y, and P;
each M₂₂ is, independently, an amino acid selected from the group consisting of P, K, Y, A, T, Q, S, G, D, R, C, V, I, L, and H;
each M₂₃ is, independently, an amino acid selected from the group consisting of T, P, F, S, A, E, G, C, R, Y, M, V, W, I, L, Q, N, D, H, and K;
each M₂₄ is, independently, an amino acid selected from the group consisting of S, T, A, N, R, G, E, P, V, F, L, Q, K, H, D, I, C, Y, M, and W;
each M₂₅ is, independently, an amino acid selected from the group consisting of F, W, Y, and P;
each M₂₆ is, independently, an amino acid selected from the group consisting of T, P, F, Q, N, S, A, E, G, D, K, Y, C, V, I, L, and H;
each M₂₇ is, independently, an amino acid selected from the group consisting of D, E, Q, N, S, T, R, K, G, A, Y, P, V, and F;
each M₂₈ is, independently, an amino acid selected from the group consisting of T, Q, N, S, A, G, C, R, K, P, Y, M, V, I, L, F, E, W, D, and H;
each M₂₉ is, independently, an amino acid selected from the group consisting of S, T, E, A, P, V, F, L, N, R, G, Q, K, H, D, I, C, Y, M, and W;
each M₃₀ is, independently, an amino acid selected from the group consisting of D, Q, N, H, K, G, C, and Y;
each M₃₁ is, independently, an amino acid selected from the group consisting of F, L, W, Y, and P;
each M₃₂ is, independently, an amino acid selected from the group consisting of S, T, E, A, P, V, F, L, N, R, G, Q, K, H, D, I, C, Y, M, and W;
each M₃₃ is, independently, an amino acid selected from the group consisting of A, G, S, Q, N, K, D, T, C, Y, E, H, V, W, I, L, F, P, R, and M;
each M₃₄ is, independently, an amino acid selected from the group consisting of T, A, V, I, P, F, Q, N, S, E, G, D, K, Y, C, L, and H;
each M₃₅ is, independently, an amino acid selected from the group consisting of G, S, R, N, H, D, P, A, T, Q, E, C, Y, V, I, L, W, F, K, and M;
each M₃₆ is, independently, an amino acid selected from the group consisting of T, Q, S, A, E, D, K, H, P, Y, V, W, I, F, L, N, G, and C;
each M₃₇ is, independently, an amino acid selected from the group consisting of I, L, W, V, and M;
each M₃₈ is, independently, an amino acid selected from the group consisting of A, G, S, Q, N, K, D, C, P, R, Y, E, V, W, T, H, M, and F;
each M₃₉ is, independently, an amino acid selected from the group consisting of S, T, E, P, V, A, F, L, N, R, G, Q, K, H, D, I, C, Y, M, and W;
each M₄₀ is, independently, an amino acid selected from the group consisting of T, S, A, D, P, M, Q, E, K, H, Y, V, W, I, F, L, N, G, and C;
each M₄₁ is, independently, an amino acid selected from the group consisting of L, F, I, V, Y, A, T, Q, S, D, M, N, K, P, E, R, H, G, and C;
each M₄₂ is, independently, an amino acid selected from the group consisting of P, Y, A, T, Q, S, N, W, G, I, E, D, L, K, and H;
each M₄₃ is, independently, an amino acid selected from the group consisting of S, E, P, V, T, A, F, L, N, R, G, Q, K, H, D, I, C, Y, M, and W;
each M₄₄ is, independently, an amino acid selected from the group consisting of N, Q, S, E, D, T, H, K, G, A, P, W, and F;
each M₄₅ is, independently, an amino acid selected from the group consisting of V, I, L, F, C, A, and T;
each M₄₆ is, independently, an amino acid selected from the group consisting of A, T, S, N, R, Y, K, D, H, M, L, F, G, Q, C, P, E, V, and W;
each M₄₇ is, independently, an amino acid selected from the group consisting of I, L, and V;
each M₄₈ is, independently, an amino acid selected from the group consisting of S, P, V, E, T, A, F, L, N, R, G, Q, K, H, D, I, C, Y, M, and W;
each M₄₉ is, independently, an amino acid selected from the group consisting of F, V, A, T, Q, N, S, E, G, D, and H;
each M₅₀ is, independently, an amino acid selected from the group consisting of L, F, I, V, Y, A, T, Q, S, D, M, N, K, P, E, R, H, G, and C;
each M₅₁ is, independently, an amino acid selected from the group consisting of G, S, R, H, D, P, N, A, T, Q, E, C, Y, V, I, L, W, F, K, and M;
each M₅₂ is, independently, an amino acid selected from the group consisting of T, N, S, G, C, R, H, A, D, P, M, Q, E, K, Y, V, W, I, F, and L;
each M₅₃ is, independently, an amino acid selected from the group consisting of I, L, W, V, and M;
each M₅₄ is, independently, an amino acid selected from the group consisting of P, K, Y, A, T, Q, S, G, D, R, C, V, I, L, and H;
each M₅₅ is, independently, an amino acid selected from the group consisting of D, E, Q, N, S, K, G, A, Y, P, F, T, R, and V;
each M₅₆ is, independently, an amino acid selected from the group consisting of L, F, I, V, Y, P, A, T, Q, N, S, G, E, D, K, H, M, C, and R;
each M₅₇ is, independently, an amino acid selected from the group consisting of S, P, V, E, T, A, F, L, N, R, G, Q, K, H, D, I, C, Y, M, and W;
each M₅₈ is, independently, an amino acid selected from the group consisting of P, M, V, I, L, and F;
each M₅₉ is, independently, an amino acid selected from the group consisting of N, Q, S, E, D, T, R, K, G, A, and Y;
each M₆₀ is, independently, an amino acid selected from the group consisting of G, S, H, P, R, D, N, A, T, Q, E, C, Y, V, I, L, W, F, K, and M;
each M₆₁ is, independently, an amino acid selected from the group consisting of S, P, V, T, A, R, K, E, H, C, Y, I, F, L, N, Q, G, D, M, and W;
each M₆₂ is, independently, an amino acid selected from the group consisting of P, K, A, Y, T, Q, S, G, D, R, C, V, I, L, and H;
each M₆₃ is, independently, an amino acid selected from the group consisting of A, G, S, N, E, K, D, H, M, V, W, I, L, F, T, R, Y, Q, C, and P;
each M₆₄ is, independently, an amino acid selected from the group consisting of D, E, Q, T, K, P, F, N, S, G, A, Y, R, and V;
each M₆₅ is, independently, an amino acid selected from the group consisting of L, V, F, I, Y, P, A, T, Q, N, S, G, E, D, K, H, M, C, and R;
each M₆₆ is, independently, an amino acid selected from the group consisting of S, N, R, T, G, K, E, H, D, A, P, V, C, Y, I, F, L, Q, M, and W;
each M₆₇ is, independently, an amino acid selected from the group consisting of K, R, H, S, G, N, Q, D, E, T, A, C, P, Y, M, V, W, I, L, and F;
each M₆₈ is, independently, an amino acid selected from the group consisting of R, K, H, S, G, N, Q, D, E, T, A, C, P, Y, M, V, W, I, L, and F;
each M₆₉ is, independently, an amino acid selected from the group consisting of S, A, N, Q, R, T, G, K, E, H, D, A, C, P, Y, M, V, W, I, F, and L; and
each M₇₀ is, independently, an amino acid selected from the group consisting of T, Q, N, S, A, E, G, D, C, R, K, H, P, Y, M, V, W, I, F, and L; and
   wherein Formula XV is given by:

(N₁)_{b}-(N₂)_{b}-(N₃)_{b}-(N₄)_{b}-(N₅)_{b}-(N₆)_{b}-(N₇)_{b}-(N₈)_{b}-(N₉)_{b}-(N₁₀)_{b}-(N₁₁)_{b}- (N₁₂)_{b}-(N₁₃)_{b}-(N₁₄)_{b}-(N₁₅)_{b}-(N₁₆)_{b}-(N₁₇)_{b}-(N₁₈)_{b}-(N₁₉)_{b}-(N₂₀)_{b}-(N₂₁)_{b}-(N₂₂)_{b}-(N₂₃)_{b}-(N₂₄)_{b}-(N₂₅)_{b}-(N₂₆)_{b}-(N₂₇)_{b}-(N₂₈)_{b}-(N₂₉)_{b}-(N₃₀)_{b}-(N₃₁)_{b}-(N₃₂)_{b}-(N₃₃)_{b}-(N₃₄)_{b}-(N₃₅)_{b}-(N₃₆)_{b}-(N₃₇)_{b}-(N₃₈)_{b}-(N₃₉)_{b}-(N₄₀)_{b}-(N₄₁)_{b}-(N₄₂)_{b}-(N₄₃)_{b}-(N₄₄)_{b}-(N₄₅)_{b}-(N₄₆)_{b}-(N₄₇)_{b}-(N₄₈)_{b}-(N₄₉)_{b}-(N₅₀)_{b}-(N₅₁)_{b}-(N₅₂)_{b}-(N₅₃)_{b}-(N₅₄)_{b}-(N₅₅)_{b}-(N₅₆)_{b}-(N₅₇)_{b}-(N₅₈)_{b}-(N₅₉)_{b}-(N₆₀)_{b}-(N₆₁)_{b}-(N₆₂)_{b}-(N₆₃)_{b}-(N₆₄)_{b}-(N₆₅)_{b}-(N₆₆)_{b}-(N₆₇)_{c}-(N₆₈)_{c}-(N₆₉)_{c}-(N₇₀)_{c}-(N₇₁)_{c} (Formula XV);

wherein:
each b is, independently, 0, 1, 2, or 3; and
each c is, independently, 1 or 2;
   wherein:
each N₁ is, independently, an amino acid selected from the group consisting of S, N, D, Q, R, T, G, E, H, A, P, M, V, K, Y, W, F, L, I, and C;
each N₂ is, independently, an amino acid selected from the group consisting of P, A, S, Y, V, T, G, I, E, and C;
each N₃ is, independently, an amino acid selected from the group consisting of T, S, G, D, C, A, L, N, R, P, Y, V, W, I, and F;
each N₄ is, independently, an amino acid selected from the group consisting of S, R, E, A, Q, K, N, D, T, G, H, C, P, Y, I, F, L, M, V, and W;
each N₅ is, independently, an amino acid selected from the group consisting of T, Q, N, G, C, M, S, A, E, D, Y, V, I, F, L, and W;
each N₆ is, independently, an amino acid selected from the group consisting of I, V, L, F, W, Y, A, T, S, E, D, and H;
each N₇ is, independently, an amino acid selected from the group consisting of P, V, A, S, N, G, E, L, and K;
each N₈ is, independently, an amino acid selected from the group consisting of A, G, Q, T, S, N, P, R, D, V, K, C, Y, W, I, L, and F;
each N₉ is, independently, an amino acid selected from the group consisting of F, Y, A, T, N, and R;
each N₁₀ is, independently, an amino acid selected from the group consisting of T, Q, N, R, K, M, S, E, D, H, P, V, W, I, F, and L;
each N₁₁ is, independently, an amino acid selected from the group consisting of A, G, Q, T, S, N, P, R, D, V, K, C, Y, W, I, L, and F;
each N₁₂ is, independently, an amino acid selected from the group consisting of S, N, Q, R, T, G, K, E, H, D, A, P, L, M, V, Y, W, F, I, and C;
each N₁₃ is, independently, an amino acid selected from the group consisting of L, F, I, W, V, M, Y, C, A, T, Q, N, S, G, E, D, and R;
each N₁₄ is, independently, an amino acid selected from the group consisting of V, I, L, A, T, S, G, R, P, Y, N, H, C, M, F, Q, E, K, and D;
each N₁₅ is, independently, an amino acid selected from the group consisting of S, N, Q, T, G, K, E, H, D, A, C, P, Y, I, F, L, R, M, V, and W;
each N₁₆ is, independently, an amino acid selected from the group consisting of T, N, S, A, D, R, P, Y, V, W, I, F, and L;
each N₁₇ is, independently, an amino acid selected from the group consisting of S, N, Q, R, K, E, D, A, T, G, H, C, P, Y, I, F, L, M, V, and W;
each N₁₈ is, independently, an amino acid selected from the group consisting of V, A, T, S, G, R, W, I, C, L, F, E, D, K, P, Y, N, H, M, and Q;
each N₁₉ is, independently, an amino acid selected from the group consisting of T, Q, N, S, A, E, G, D, Y, M, V, I, F, L, and W;
each N₂₀ is, independently, an amino acid selected from the group consisting of S, Q, R, K, E, A, N, D, T, G, H, C, P, Y, I, F, L, M, V, and W;
each N₂₁ is, independently, an amino acid selected from the group consisting of V, W, I, C, L, F, A, T, S, E, D, K, G, R, P, Y, N, H, M, and Q;
each N₂₂ is, independently, an amino acid selected from the group consisting of T, Q, N, S, A, D, C, K, P, Y, M, V, W, I, F, G, E, H, R, and L;
each N₂₃ is, independently, an amino acid selected from the group consisting of L, F, I, V, P, A, T, Q, S, G, R, K, H, M, Y, and D;
each N₂₄ is, independently, an amino acid selected from the group consisting of T, Q, S, A, G, P, Y, I, K, H, V, F, L, N, D, C, M, W, E, and R;
each N₂₅ is, independently, an amino acid selected from the group consisting of S, R, E, A, Q, K, N, D, T, G, H, C, P, Y, I, F, L, M, V, and W;
each N₂₆ is, independently, an amino acid selected from the group consisting of T, N, D, S, A, R, P, Y, V, W, I, F, and L;
each N₂₇ is, independently, an amino acid selected from the group consisting of D, N, R, E, Q, S, H, T, K, G, W, I, P, and Y;
each N₂₈ is, independently, an amino acid selected from the group consisting of V, A, T, S, G, R, W, I, C, L, F, E, D, K, P, Y, N, H, M, and Q;
each N₂₉ is, independently, an amino acid selected from the group consisting of T, S, A, D, C, L, N, R, P, Y, V, W, I, and F;
each N₃₀ is, independently, an amino acid selected from the group consisting of P, Y, V, A, T, S, G, I, E, and C;
each N₃₁ is, independently, an amino acid selected from the group consisting of T, Q, S, A, G, K, H, P, Y, V, I, F, L, N, D, C, M, W, E, and R;
each N₃₂ is, independently, an amino acid selected from the group consisting of S, R, E, A, Q, K, N, D, T, G, H, C, P, Y, I, F, L, M, V, and W;
each N₃₃ is, independently, an amino acid selected from the group consisting of E, D, Q, N, S, T, H, R, G, A, P, F, and L;
each N₃₄ is, independently, an amino acid selected from the group consisting of D, N, R, E, Q, S, H, T, K, G, W, I, P, and Y;
each N₃₅ is, independently, an amino acid selected from the group consisting of T, Q, S, A, G, P, Y, I, K, H, V, F, L, N, D, C, M, W, E, and R;
each N₃₆ is, independently, an amino acid selected from the group consisting of G, S, K, A, T, Q, D, C, P, Y, V, W, I, L, and F;
each N₃₇ is, independently, an amino acid selected from the group consisting of F, Y, A, T, N, and R;
each N₃₈ is, independently, an amino acid selected from the group consisting of V, A, T, S, G, R, W, I, C, L, F, E, D, K, P, Y, N, H, M and Q;
each N₃₉ is, independently, an amino acid selected from the group consisting of L, F, I, W, V, M, C, A, T, Q, N, S, G, D, R, K, and H;
each N₄₀ is, independently, an amino acid selected from the group consisting of P, A, S, Y, V, T, G, I, E, and C;
each N₄₁ is, independently, an amino acid selected from the group consisting of D, N, R, G, Y, E, Q, S, H, T, K, W, and I;
each N₄₂ is, independently, an amino acid selected from the group consisting of S, R, E, A, N, T, G, P, V, Q, K, H, D, Y, M, I, F, L, C, and W;
each N₄₃ is, independently, an amino acid selected from the group consisting of G, S, R, K, A, N, Q, H, E, D, P, W, L, and F;
each N₄₄ is, independently, an amino acid selected from the group consisting of T, Q, S, A, G, P, Y, I, N, E, D, C, K, H, R, V, L, M, F, and W;
each N₄₅ is, independently, an amino acid selected from the group consisting of S, T, G, A, V, I, R, E, N, P, Q, K, H, D, Y, M, F, L, C, and W;
each N₄₆ is, independently, C;
each N₄₇ is, independently, an amino acid selected from the group consisting of S, N, R, T, G, K, E, H, D, A, P, Y, V, W, I, L, Q, M, F, and C;
each N₄₈ is, independently, an amino acid selected from the group consisting of G, S, R, K, N, T, Q, H, E, D, P, I, and L;
each N₄₉ is, independently, an amino acid selected from the group consisting of T, S, G, D, C, A, L, N, R, P, Y, V, W, I, and F;
each N₅₀ is, independently, an amino acid selected from the group consisting of V, A, T, S, G, I, R, P, Y, L, N, H, C, M, F, Q, E, and K;
each N₅₁ is, independently, an amino acid selected from the group consisting of A, T, G, S, Q, N, R, Y, E, H, M, V, W, I, L, and F;
each N₅₂ is, independently, an amino acid selected from the group consisting of D, E, Q, N, S, T, K, A, Y, P, M, W, I, F, and L;
each N₅₃ is, independently, an amino acid selected from the group consisting of A, T, C, G, S, N, P, R, K, D, H, M, and F;
each N₅₄ is, independently, an amino acid selected from the group consisting of L, F, I, V, P, A, T, Q, S, G, R, K, H, M, Y, and D;
each N₅₅ is, independently, an amino acid selected from the group consisting of E, D, N, T, R, K, G, A, and V;
each N₅₆ is, independently, an amino acid selected from the group consisting of A, G, Q, T, S, N, P, R, D, V, W, K, C, Y, I, L, and F;
each N₅₇ is, independently, an amino acid selected from the group consisting of Y, C, N, I, F, and L;
each N₅₅ is, independently, an amino acid selected from the group consisting of S, T, G, H, A, P, Y, V, F, L, N, R, K, E, D, W, I, Q, M, and C;
each N₅₉ is, independently, an amino acid selected from the group consisting of I, V, and L;
each N₆₀ is, independently, S
each N₆₁ is, independently, an amino acid selected from the group consisting of G, S, R, K, A, N, T, Q, E, D, P, and Y;
each N₆₂ is, independently, an amino acid selected from the group consisting of I, V, L, F, W, Y, A, T, S, E, D, and H;
each N₆₃ is, independently, an amino acid selected from the group consisting of T, Q, N, G, C, M, S, A, E, D, Y, V, I, F, L, and W;
each N₆₄ is, independently, an amino acid selected from the group consisting of S, N, Q, R, G, K, E, D, P, Y, W, F, T, H, A, V, L, I, M, and C;
each N₆₅ is, independently, an amino acid selected from the group consisting of A, C, G, S, Q, N, R, Y, E, K, D, H, M, V, I, and L;
each N₆₆ is, independently, an amino acid selected from the group consisting of V, I, A, T, S, G, R, P, Y, L, N, H, C, M, F, Q, E, K, and D;
each N₆₇ is, independently, an amino acid selected from the group consisting of S, N, Q, R, T, G, K, E, H, D, A, C, P, Y, M, V, W, I, F, and L;
each N₆₈ is, independently, an amino acid selected from the group consisting of K, R, H, S, G, N, Q, D, E, T, A, C, P, Y, M, V, W, I, L, and F;
each N₆₉ is, independently, an amino acid selected from the group consisting of K, R, H, S, G, N, Q, D, E, T, A, C, P, Y, M, V, W, I, L, and F;
each N₇₀ is, independently, an amino acid selected from the group consisting of D, E, Q, N, S, H, T, R, K, G, A, C, Y, P, M, V, W, I, F, and L;
each N₇₁ is, independently, an amino acid selected from the group consisting of A, T, C, G, S, Q, N, P, R, Y, E, K, D, H, M, V, W, I, L, and F.
   13. The pro-protein signal peptide of embodiment 12, wherein the signal peptide comprises an amino acid sequence having at least 90%, 91%, 92%, 93%, 94%, 95%, 96%, 97%, 98% or 99% identity to an amino acid sequence selected from the group consisting of SEQ ID NO. 17, 18, 19, 20, 21, 22, 23, 24, 25, 27, 29, 34, 35, 36, 37, 38, 56, 57, 58, 74, or 75.
   14. The pro-protein signal peptide of embodiment 12, wherein the amino acid sequence is selected from the group consisting of SEQ ID NO. 17, 18, 19, 20, 21, 22, 23, 24, 25, 27, 29, 34, 35, 36, 37, 38, 56, 57, 58, 74, or 75.
   15. A pro-protein signal peptide comprising an amino acid sequence having at least 90%, 91%, 92%, 93%, 94%, 95%, 96%, 97%, 98%, 99% or 100% identity to an amino acid sequence selected from the group consisting of SEQ ID NO. 17, 18, 19, 20, 21, 22, 23, 24, 25, 27, 29, 34, 35, 36, 37, 38, 56, 57, 58, 74, or 75.
   16. A polypeptide comprising a formula of (X₁)ₙ-(Y₁)ₘ-Z₁ wherein:
X₁ is a pre-protein signal peptide,
Y₁ is a pro-protein signal peptide, and
Z₁ is a payload protein,
wherein n is 0-1 and m is 0-1,
wherein n and m cannot concurrently be 0.
   17. The polypeptide of embodiment 16, wherein n is 1 and X₁ comprises an amino acid sequence selected from the group consisting of Formula I, Formula II, Formula III, Formula IV, Formula V, Formula IX, and Formula XIII wherein Formula I is given by:

A₁-(A₂)_{w}-A₃-(A₄)ₓ-(A₅)_{y}-A₆-A₇-A₈-A₉-A₁₀-(A₁₁)_{z} (Formula I)

wherein:
w and x are each, independently, 1, 2, 3, 4, or 5;
y is 2, 3, 4, 5, 6, 7, 8, 9, 10, 11, 12, 13, 14, 15, 16, 17, 18, 19, or 20; and
z is 1, 2, or 3;
   wherein:
A₁ is methionine;
each A₂ is, independently, a neutral or positively-charged amino acid with a hydropathy index of less than about 1;
each A₃, A₅, A₈, and A₁₀ is each, independently, an amino acid with a hydropathy index greater than −1, excluding W and C;
each A₄ is, independently, a basic or neutral amino acid, excluding P, W, M, and C;
A₆ is an amino acid with a hydropathy index greater than −1, excluding W, M, and C;
A₇ is a non-aromatic amino acid with a hydropathy index of less than about 1.9 and an isoelectric point of about 5.4 to about 7.5, excluding P;
A₉ is an amino acid with a hydropathy index of greater than about −1.3; and
each A₁₁ is, independently, a neutral amino acid with a molecular weight of less than about 133 g/mol;
   wherein Formula II is given by:

B₁-(B₂)ᵤ-(B₃)ᵥ-(B₄)_{w}-(B₅)ₓ-(B₆)_{y}-B₇-B₈-B₉-B₁₀-(B₁₁)_{z} (Formula II)

wherein:
u and w are each, independently, 0, 1, 2, or 3;
v and z are each, independently, 1, 2, or 3;
x is 0, 1, or 2; and
y is 2, 3, 4, 5, 6, 7, 8, 9, 10, 11, 12, 13, 14, 15, 16, 17, 18, 19, or 20;
   wherein:
B₁ is methionine;
each B₂, B₄, B₆, B₈ and B₁₀ is each, independently, an amino acid with a hydropathy index of greater than about −1, excluding W and C;
each B₃ is, independently, a positively-charged or polar amino acid with a hydropathy index of less than about 1;
each B₅ is, independently, a polar amino acid with a hydropathy index of greater than about −5 and less than about −0.5, or an amino acid with an isoelectric point of about 5 to about 11, excluding P, W, M, and C;
each B₇ and B₁₁ is each, independently, a neutral amino acid with a molecular weight of less than about 133 g/mol; and
B₉ is an amino acid with a hydropathy index of greater than about −1.3;
   wherein Formula III is given by:

C₁-(C₂)ᵣ-(C₃)ₜ-(C₄)ᵤ-[(C₅)ᵥ-(C₆)_{w}]ₓ-(C₇)_{y}-(C₈)_{z}-C₉-C₁₀-C₁₁-[C₁₂-C₁₃]ₐ (Formula III)

wherein:
r is 1, 2, or 3;
t, u, y, and z are each, independently, 0, 1, 2, or 3;
v and w are each, independently, 0, 1, or 2;
a is 0 or 1; and
x is 2, 3, 4, 5, 6, 7, 8, 9, or 10;
   wherein:
C₁ is methionine;
each C₂ is, independently, an amino acid having an isoelectric point of about 5.6 to about 10.8, a molecular weight of about 105 g/mol to about 175 g/mol, a hydropathy index of about −5.1 to about 0.6, and a helicity of about 0.8 to about 1;
each C₃, C₅, C₈, and C₁₀ is each, independently, an amino acid having an isoelectric point of about 2.75 to about 10.8, a molecular weight of about 75 g/mol to about 205 g/mol, a hydropathy index of about −5.1 to about 34, and a helicity of about 0.5 to about 1.3;
each C₄ and C₇ is each, independently, an amino acid having an isoelectric point of about 5 to about 10.8, a molecular weight of about 75 g/mol to about 205 g/mol, a hydropathy index of about −5.1 to about 34, and a helicity of about 0.5 to about 1.3;
each C₆, C₉, C₁₁, and C₁₂ is each, independently, an amino acid having an isoelectric point of about 2.75 to about 9.8, a molecular weight of about 75 g/mol to about 205 g/mol; a hydropathy index of about −4 to about 34, and a helicity of about 0.5 to about 1.3; and
C₁₃ is an amino acid having an isoelectric point of about 5.6 to about 6.3, a molecular weight of about 105 g/mol to about 120 g/mol, a hydropathy index of about 0 to about 9.4, and a helicity of about 0.5 to about 1.1;
   wherein Formula IV is given by:

D₁-(D₂)_{q}-(D₃)ᵣ-(D₄)ₜ-(D₅)ᵤ-[(D₆)ᵥ-(D₇)ₓ-(D₈)_{w}-(D₉)_{y}]_{z}-D₁₀-D₁₁-D₁₂-[D₁₃-D₁₄]ₐ (Formula IV)

wherein:
q is 1, 2, or 3;
r, t, and u are each, independently, 0, 1, 2, or 3;
v, w, x, and y are each, independently, 0, 1, or 2;
a is 0 or 1; and
z is 2, 3, 4, 5, 6, 7, 8, 9, or 10;
   wherein:
D₁ is methionine;
each D₂ is, independently, an amino acid having an isoelectric point of about 2.7 to about 10.8, a molecular weight of about 75 g/mol to about 205 g/mol, a hydropathy index of about −5.1 to about 34, and a helicity of about 0.5 to about 1.3;
each D₃ is, independently, an amino acid having an isoelectric point of about 5 to about 10.8, a molecular weight of about 89 g/mol to about 205 g/mol, a hydropathy index of about −4 to about 34, and a helicity of about 0.5 to about 1.3;
each D₄, D₉ and D₁₁ is each, independently an amino acid having an isoelectric point of about 2.7 to about 10.8, a molecular weight of about 75 g/mol to about 205 g/mol, a hydropathy index of about −5.1 to about 34, and a helicity of about 0.5 to about 1.3;
each D₅ is, independently, an amino acid having an isoelectric point of about 3.2 to about 10.8, a molecular weight of about 75 g/mol to about 205 g/mol, a hydropathy index of about −5.1 to about 34, and a helicity of about 0.75 to about 1.3;
each D₆ is, independently, an amino acid having an isoelectric point from about 5 to about 10.8, a molecular weight of about 75 g/mol to about 205 g/mol, a hydropathy index of about −5.1 to about 34, and a helicity of about 0.5 to about 1.3;
each D₇ is, independently, an amino acid having an isoelectric point of about 5.4 to about 6.1, a molecular weight of about 117 g/mol to about 205 g/mol, a hydropathy index of about 2.5 to about 34, and a helicity of about 1 to about 1.3;
each D₈, D₁₀, D₁₂, and D₁₃ is each, independently, an amino acid having an isoelectric point of about 2.7 to about 10.8, a molecular weight of about 75 g/mol to about 182 g/mol, a hydropathy index of about −5.1 to about 32, and a helicity of about 0.75 to about 1.3; and
D₁₄ is an amino acid with an isoelectric point of about 2.7 to about 10.8, a molecular weight of about 75 g/mol to about 182 g/mol, a hydropathy index of about −5.1 to about 32, and a helicity of about 0.5 to about 1.3;
   wherein Formula V is given by:

E₁-[(E₂)ᵢ-(E₃)ⱼ-(E₄)_{q}]ᵣ-(E₅)ₜ-(E₆)ᵤ-(E₇)ᵥ-[(E₈)_{w}-(E₉)ₓ]_{y}-(E₁₀)_{z}-E₁₁-E₁₂-E₁₃-[E₁₄-E₁₅]ₐ (Formula V)

wherein:
i, j, q, w, x and a are each, independently, 0 or 1;
r is 1, 2, or 3;
t, u, v, and z are each, independently, 0, 1, 2, or 3; and
y is 2, 3, 4, 5, 6, 7, 8, 9, or 10;
   wherein:
E₁ is methionine;
each E₂ is, independently, an amino acid having an isoelectric point of about 3.2 to about 10.8, a molecular weight of about 105 g/mol to about 175 g/mol, a hydropathy index of about −4 to about 1, and a helicity of about 0.85 to about 1;
each E₃ is, independently, an amino acid having an isoelectric point of about 2.7 to about 10.8, a molecular weight of about 75.1 g/mol to about 205 g/mol, a hydropathy index of about −5.1 to about 33.5, and a helicity of about 0.57 to about 1.3;
each E₄ is, independently, an amino acid having an isoelectric point of about 5 to about 10.8, a molecular weight of about 105 g/mol to about 205 g/mol, a hydropathy index of about −5.1 to about 33.5, and a helicity of about 0.57 to about 1.3;
each E₅ and E₈ is each, independently, an amino acid having an isoelectric point of about 2.7 to about 10.8, a molecular weight of about 75 g/mol to about 205 g/mol, a hydropathy index of about −5.1 to about 33.5, and a helicity of about 0.57 to about 1.3;
each E₆ is, independently, an amino acid having an isoelectric point of about 5 to about 10.8, a molecular weight of about 89 g/mol to about 205 g/mol, a hydropathy index of about −5.1 to about 33.5, and a helicity of about 0.57 to about 1.3;
each E₇ is, independently, an amino acid having an isoelectric point of about 5 to about 9.75, a molecular weight of about 75 g/mol to about 205 g/mol, a hydropathy index of about −4 to about 33.5, and a helicity of about 0.79 to about 1.3;
each E₉, E₁₃, and E₁₄ is each, independently, an amino acid having an isoelectric point of about 2.7 to about 10.8, a molecular weight of about 75 g/mol to about 205 g/mol, a hydropathy index of about −5.1 to about 33.5, and a helicity of about 0.57 to about 1.3;
each E₁₀ and E₁₂ is, independently, an amino acid having an isoelectric point of about 5 to about 10.8, a molecular weight of about 75 g/mol to about 205 g/mol, a hydropathy index of about −5.1 to about 34, and a helicity of about 0.5 to about 1.3;
E₁₁ is an amino acid having an isoelectric point of about 5 to about 9.75, a molecular weight of about 89 g/mol to about 205 g/mol, a hydropathy index of about −4 to about 33.5, and a helicity of about 0.79 to about 1.3; and
E₁₅ is an amino acid having an isoelectric point of about 2.7 to about 10.8, a molecular weight of about 75 g/mol to about 205 g/mol, a hydropathy index of about −4 to about 15.5, and a helicity of about 0.57 to about 1.2;
   wherein Formula IX is given by:

F₁-(F₂)ᵥ-(F₃)_{w}-[(F₄)ₓ-(F₅)_{y}]_{z}-F₆-F₇-F₈-[F₉-F₁₀]ₐ (Formula IX)

wherein:
v and w are each, independently, 0, 1, 2, or 3;
x and y are each, independently, 0, 1, 2, 3, or 4;
a is 0 or 1; and
z is 1, 2, 3, 4, 5, 6, 7, or 8;
   wherein:
F₁ is an amino acid having an isoelectric point of about 5.4 to about 11, a molecular weight of about 89 g/mol to about 175 g/mol; a hydropathy index of about −4 to about 31, and a helicity or about 0.9 to about 1.3;
each F₂ is, independently, an amino acid having an isoelectric point of about 3 to about 11, a molecular weight of about 89 g/mol to about 205 g/mol; a hydropathy index of about −5.1 to about 34, and a helicity or about 0.5 to about 1.3;
each F₃ and F₇ is each, independently, an amino acid having an isoelectric point of about 3 to about 11, a molecular weight of about 89 g/mol to about 205 g/mol; a hydropathy index of about −5.1 to about 34, and a helicity or about 0.5 to about 1.3;
each F₄ is, independently, an amino acid having an isoelectric point of about 3 to about 11, a molecular weight of about 89 g/mol to about 205 g/mol; a hydropathy index of about −5.1 to about 34, and a helicity or about 0.5 to about 1.3;
each F₅, F₆, F₈, and F₉ is each, independently, an amino acid having an isoelectric point of about 3 to about 11, a molecular weight of about 89 g/mol to about 205 g/mol; a hydropathy index of about −5.1 to about 34, and a helicity or about 0.5 to about 1.3; and
F₁₀ is an amino acid having an isoelectric point of about 3 to about 11, a molecular weight of about 89 g/mol to about 205 g/mol; a hydropathy index of about −5.1 to about 34, and a helicity or about 0.5 to about 1.3; and
   wherein Formula XIII is given by:

L₁-(L₂)ₓ-[(L₃)ₐ-(L₄)ₐ]_{y}-[(L₅)ₐ-(L₆)ₐ-(L₇)ₐ]_{z}-(L₈)ₐ-(L₉)ₐ-(L₁₀)ₐ-(L₁₁)ₐ-(L₁₂)ₐ (Formula XIII)

wherein:
x is 1, 2, or 3;
y is 1, 2, 3, or 4;
z is 5, 6, 7, 8, 9, or 10; and
each a is, independently, 0 or 1;
   wherein:
each L₂ is, independently, an amino acid having an isoelectric point of about 2.7 to about 10.8, a molecular weight of about 75 g/mol to about 205 g/mol; a hydropathy index of about −5.1 to about 34, and a helicity or about 0.5 to about 1.3;
each L₃ and L₆ is each, independently, an amino acid having an isoelectric point of about 2.7 to about 10.8, a molecular weight of about 75 g/mol to about 205 g/mol; a hydropathy index of about −5.1 to about 34, and a helicity or about 0.5 to about 1.3;
each L₄, L₇ and L₉ is each, independently, an amino acid having an isoelectric point of about 2.7 to about 10.8, a molecular weight of about 75 g/mol to about 205 g/mol; a hydropathy index of about −5.1 to about 34, and a helicity or about 0.5 to about 1.3;
each L₅, L₈, L₁₀ and L₁₁ is each, independently, an amino acid having an isoelectric point of about 2.7 to about 10.8, a molecular weight of about 75 g/mol to about 205 g/mol; a hydropathy index of about −5.1 to about 34, and a helicity or about 0.5 to about 1.3; and
L₁₂ is an amino acid having an isoelectric point of about 2.7 to about 10.8, a molecular weight of about 75 g/mol to about 205 g/mol; a hydropathy index of about −5.1 to about 34, and a helicity or about 0.5 to about 1.3.
   18. The polypeptide of embodiment 16 or 17 wherein n is 1 and X₁ comprises an amino acid sequence having at least 90%, 91%, 92%, 93%, 94%, 95%, 96%, 97%, 98%, 99%, or 100% identity to an amino acid sequence selected from the group consisting of SEQ ID NO. 1, 2, 3, 4, 5, 6, 7, 8, 9, 10, 11, 12, 13, 14, 15, 16, 28, 31, 32, 33, 55, 70, 71, 72, or 73.
   19. The polypeptide of any one of embodiments 16-18, wherein m is 1 and Y₁ comprises an amino acid sequence selected from the group consisting of Formula VI, Formula VII, Formula VIII, Formula X, Formula XI, Formula XIV, and Formula XV, wherein Formula VI is given by:

G₁-G₂-G₃-G₄-G₅-G₆-G₇-G₈-G₉-G₁₀-G₁₁-G₁₂-G₁₃-G₁₄-G₁₅-G₁₆-G₁₇-G₁₈-G₁₉-G₂₀-G₂₁-G₂₂-G₂₃-G₂₄-G₂₅ (Formula VI)

wherein:
G₁ is an amino acid selected from the group consisting of I, L, F, V, A, N, S, D, R, and K;
G₂ is an amino acid selected from the group consisting of P, S, N, G, and E;
G₃ is an amino acid selected from the group consisting of L, F, I, V, Y, A, S, R, and H;
G₄ is an amino acid selected from the group consisting of V, M, P, Y, A, T, S, N, K, and H;
G₅ is an amino acid selected from the group consisting of A, G, R, Y, K, D, M, V, W, I, and L;
G₆ is an amino acid selected from the group consisting of N, R, and K;
G₇ is an amino acid selected from the group consisting of V, P, A, T, Q, G, E, D, R, and K;
G₈ is an amino acid selected from the group consisting of P, Y, T, Q, S, N, W, F, R, K, and H;
G₉ is an amino acid selected from the group consisting of F, L, A, Q, N, S, E, G, D, and H;
G₁₀ is an amino acid selected from the group consisting of H, S, N, D, Q, E, T, Y, M, V, I, and L;
G₁₁ is an amino acid selected from the group consisting of S, R, T, G, K, E, D, and P;
G₁₂ is an amino acid selected from the group consisting of D, E, Q, N, A, and V;
G₁₃ is an amino acid selected from the group consisting of N, S, E, D, T, H, K, A, and P;
G₁₄ is an amino acid selected from the group consisting of G, S, N, H, E, C, Y, L, and F;
G₁₅ is an amino acid selected from the group consisting of S, T, and H;
G₁₆ is an amino acid selected from the group consisting of E, D, Q, N, S, T, K, and A;
G₁₇ is an amino acid selected from the group consisting of W, N, D, and R;
G₁₈ is an amino acid selected from the group consisting of L and F;
G₁₉ is an amino acid selected from the group consisting of Y, V, A, Q, N, S, E, D, L, R, K, and H;
G₂₀ is an amino acid selected from the group consisting of K, R, S, and I;
G₂₁ is R;
G₂₂ is an amino acid selected from the group consisting of D, E, N, S, T, G, A, Y, and L;
G₂₃ and G₂₄ are each, independently, an amino acid selected from the group consisting of V, P, Y, I, A, E, K, F, T, S, G, D, M, and N; and
G₂₅ is an amino acid selected from the group consisting of Y, P, A, T, Q, S, E, F, and H;
   wherein Formula VII is given by:

(H₁)ₘ-(H₂)ₘ-(H₃)ₘ-(H₄)ₘ-(H₅)ₘ-(H₆)ₘ-(H₇)ₘ-(H₈)ₘ-(H₉)ₘ-(H₁₀)ₘ-(H₁₁)ₘ- (H₁₂)ₘ-(H₁₃)ₘ-(H₁₄)ₘ-(H₁₅)ₘ-(H₁₆)ₘ-(H₁₇)ₘ-(H₁₈)ₘ-(H₁₉)ₘ-(H₂₀)ₘ-(H₂₁)ₘ-(H₂₂)ₘ-(H₂₃)ₘ-(H₂₄)ₘ-(H₂₅)ₘ-(H₂₆)ₘ-(H₂₇)ₘ-(H₂₈)ₘ-(H₂₉)ₘ-(H₃₀)ₘ-(H₃₁)ₘ-(H₃₂)ₘ-(H₃₃)ₘ-(H₃₄)ₘ-(H₃₅)ₘ-(H₃₆)ₘ-H₃₇-H₃₈-H₃₉-H₄₀ (Formula VII)

wherein:
each m is, independently, 0, 1, or 2;
   wherein:
each H₁ is, independently, an amino acid selected from the group consisting of E, D, S, L, G, Q, and A;
each H₂ and H₂₈ is each, independently, an amino acid selected from the group consisting of P, S, R, T, N, G, D, K, and A;
each H₃ is, independently, an amino acid selected from the group consisting of W and Y;
each H₄ is, independently, an amino acid selected from the group consisting of S, N, A, P, and V;
each H₅ and H₃₀ is each, independently, an amino acid selected from the group consisting of T, Q, A, E, F, and S;
each H₆ is, independently, an amino acid selected from the group consisting of L, F, and I;
each H₇ is, independently, an amino acid selected from the group consisting of F, V, M, T, S, and K;
each H₈ is, independently, an amino acid selected from the group consisting of V, P, I, A, S, and K;
each H₉ and H₁₇ is each, independently, an amino acid selected from the group consisting of T, G, V, W, and A;
each H₁₀ is, independently, an amino acid selected from the group consisting of R, H, S, G, N, E, T, and V;
each H₁₁ is, independently, an amino acid selected from the group consisting of S, G, D, A, and M;
each H₁₂ is, independently, an amino acid selected from the group consisting of T, S, E, G, D, K, and H;
each H₁₃ is, independently, an amino acid selected from the group consisting of L, M, Y, N, S, D, and K;
each H₁₄ is, independently, an amino acid selected from the group consisting of D, Q, N, S, K, and C;
each H₁₅ is, independently, an amino acid selected from the group consisting of E, S, D, L, and G;
each H₁₆ is, independently, an amino acid selected from the group consisting of I, L, V, M, A, and T;
each H₁₈ is, independently, an amino acid selected from the group consisting of D, E, S, T, K, and G;
each H₁₉ is, independently, an amino acid selected from the group consisting of Y, F, and L;
each H₂₀ is, independently, an amino acid selected from the group consisting of N, Q, S, T, R, and F;
each H₂₁ and H₃₄ is each, independently, an amino acid selected from the group consisting of S, K, T, A, Y, M, and F;
each H₂₂ is, independently, an amino acid selected from the group consisting of T, Q, S, D, C, V, and L;
each H₂₃ is, independently, an amino acid selected from the group consisting of G, S, K, N, H, D, W, and L;
each H₂₄ is, independently, an amino acid selected from the group consisting of I, L, V, P, N, and E;
each H₂₅ and H₃₃ is each, independently, an amino acid selected from the group consisting of A, T, G, R, Y, L, F, and E;
each H₂₆ and H₄₀ is each, independently, an amino acid selected from the group consisting of V, I, F, M, L, A, and T;
each H₂₇ is, independently, an amino acid selected from the group consisting of D, E, Q, N, S, A, and I;
each H₂₉ is, independently, an amino acid selected from the group consisting of E, D, T, A, Y, M, V, I, F, and L;
each H₃₁ is, independently, an amino acid selected from the group consisting of F, W, V, M, S, G, and R;
each H₃₂ is, independently, an amino acid selected from the group consisting of H, S, E, G, and T;
each H₃₅ is, independently, an amino acid selected from the group consisting of R, K, S, and Q;
each H₃₆ is, independently, an amino acid selected from the group consisting of H, R, S, T, A, V, W, and L;
H₃₇ is an amino acid selected from the group consisting of K, Q, D, A, and I;
H₃₈ is an amino acid selected from the group consisting of R, K, T, and F; and
H₃₉ is an amino acid selected from the group consisting of D, N, S, T, K, A, Y, and L;
   wherein Formula VIII is given by:

(I₁)ₘ-(I₂)ₘ-(I₃)ₘ-(I₄)ₘ-(I₅)ₘ-(I₆)ₘ-(I₇)ₓ-(I₈)ₘ-(I₉)ₘ-(I₁₀)ₘ-(I₁₁)ₓ- (I₁₂)ₘ-(I₁₃)ₓ-(I₁₄)ₓ-(I₁₅)ₘ-(I₁₆)ₓ-(I₁₇)ₘ-I₁₈-I₁₉-I₂₀-I₂₁-I₂₂-I₂₃ (Formula VIII)

wherein:
each m is, independently, 0, 1, or 2; and
each x is, independently, 0, 1, 2, 3, or 4;
   wherein:
each I₁ and I₆ is each, independently, an amino acid selected from the group consisting of S, Q, E, A, I, G, V, R, T, and Y;
each I₂ is, independently, an amino acid selected from the group consisting of T, S, E, R, P, V, I, and F;
each I₃ is, independently, L.
each I₄ is, independently, an amino acid selected from the group consisting of T, N, K, and M;
each I₅ is, independently, an amino acid selected from the group consisting of P, A, and D;
each I₇ is, independently, an amino acid selected from the group consisting of T, S, K, H, Y, V, and F;
each I₈ and I₁₅ is each, independently, an amino acid selected from the group consisting of F, L, W, A, T, M, Y, and C;
each I₉ is, independently, an amino acid selected from the group consisting of I, L, and V;
each I₁₀ and I₁₆ is each, independently, an amino acid selected from the group consisting of G, S, N, E, D, A, K, H, C, P, and F;
each I₁₁ is, independently, an amino acid selected from the group consisting of I, L, V, A, T, and S;
each I₁₂ is, independently, an amino acid selected from the group consisting of T, N, A, E, and G;
each I₁₃ is, independently, an amino acid selected from the group consisting of E, Q, S, T, R, K, A, L, D, and F;
each I₁₄ is, independently, an amino acid selected from the group consisting of T, S, Q, F, A, G, V, I, and L;
each I₁₇ is, independently, an amino acid selected from the group consisting of I, L, V, N, A, T, and S;
I₁₈ and I₂₁ are each, independently, an amino acid selected from the group consisting of R, K, Q, and A;
I₁₉ is an amino acid selected from the group consisting of H, R, S, N, T, A, V, and W;
I₂₀ is an amino acid selected from the group consisting of K, N, Q, D, E, A, and I;
I₂₂ is an amino acid selected from the group consisting of D, N, S, A, Y, and L; and
I₂₃ is an amino acid selected from the group consisting of V, I, L, F, and A;
   wherein Formula X is given by:

(J₁)_{z}-(J₂)_{z}-(J₃)_{z}-(J₄)_{z}-(J₅)_{z}-(J₆)_{z}-(J₇)_{z}-(J₈)_{z}-(J₉)_{z}-(J₁₀)_{z}-(J₁₁)_{z}- (J₁₂)_{z}-(J₁₃)_{z}-(J₁₄)_{z}-(J₁₅)_{z}-(J₁₆)_{z}-(J₁₇)_{z}-(J₁₈)_{z}-(J₁₉)_{z}-(J₂₀)_{z}-(J₂₁)_{z}-J₂₂-J₂₃-J₂₄-J₂₅ (Formula X)

wherein:
each z is, independently, 0, 1, 2, 3, 4, or 5;
   wherein:
each J₁ is, independently, an amino acid selected from the group consisting of H, K, G, A, P, F, and L;
each J₂ is, independently, an amino acid selected from the group consisting of D, E, N, G, P, H, T, R, K, and A;
each J₃ is, independently, an amino acid selected from the group consisting of G, A, P, V, and L;
each J₄ is, independently, an amino acid selected from the group consisting of F, I, P, A, S, E, D, R, and K;
each J₅ is, independently, an amino acid selected from the group consisting of S, R, T, G, K, E, D, and C;
each J₆ is, independently, an amino acid selected from the group consisting of T, S, A, D, and F;
each J₇ is, independently, an amino acid selected from the group consisting of D, E, N, G, P, H, T, R, K, and A;
each J₈ is, independently, an amino acid selected from the group consisting of Y, C, A, W, I, S, E, D, F, L, R, and K;
each J₉ is, independently, an amino acid selected from the group consisting of H, K, N, D, G, T, A, C, Y, V, and L;
each J₁₀ is, independently, an amino acid selected from the group consisting of L, V, A, G, E, I, P, and R;
each J₁ is, independently, an amino acid selected from the group consisting of I, W, V, Y, P, T, N, S, R, and K;
each J₁₂ is, independently, an amino acid selected from the group consisting of A, G, Q, N, R, Y, E, D, and L;
each J₁₃ is, independently, an amino acid selected from the group consisting of I, L, W, V, M, Y, P, A, S, and G;
each J₁₄ is, independently, an amino acid selected from the group consisting of V, C, L, F, A, T, N, G, and R;
each J₁₅ is, independently, an amino acid selected from the group consisting of G, S, R, K, A, T, H, E, W, L, and F;
each J₁₆ is, independently, an amino acid selected from the group consisting of D, E, Q, S, H, T, R, G, Y, V, F, and L;
each J₁₇ is, independently, an amino acid selected from the group consisting of E, S, G, Y, I, and L;
each J₁₈ is, independently, an amino acid selected from the group consisting of A, S, P, H, and V;
each J₁₉ is, independently, an amino acid selected from the group consisting of N, E, R, K, and A;
each J₂₀ is, independently, an amino acid selected from the group consisting of R, T, V, I, and L;
each J₂₁ is, independently, an amino acid selected from the group consisting of L, V, A, G, E, I, P, and R;
J₂₂ is an amino acid selected from the group consisting of K, R, D, T, M, and W;
J₂₃ is an amino acid selected from the group consisting of R, T, V, I, and L;
J₂₄ is an amino acid selected from the group consisting of S, N, G, E, D, P, and W; and
J₂₅ is an amino acid selected from the group consisting of A, T, S, Y, M, V, and L;
   wherein Formula XI is given by:

(K₁)_{b}-(K₂)_{b}-(K₃)_{b}-(K₄)_{b}-(K₅)_{b}-(K₆)_{b}-(K₇)_{b}-(K₈)_{b}-(K₉)_{b}-(K₁₀)_{b}-(K₁₁)_{b}- (K₁₂)_{b}-(K₁₃)_{b}-(K₁₄)_{b}-(K₁₅)_{b}-(K₁₆)_{b}-(K₁₇)_{b}-(K₁₈)_{b}-(K₁₉)_{b}-(K₂₀)_{b}-(K₂₁)_{b}-(K₂₂)_{b}-(K₂₃)_{b}-(K₂₄)_{b}-(K₂₅)_{b}-(K₂₆)_{b}-(K₂₇)_{b}-(K₂₈)_{b}-(K₂₉)_{b}-(K₃₀)_{b}-(K₃₁)_{b}-(K₃₂)_{b}-(K₃₃)_{b}-(K₃₄)_{b}-(K₃₅)_{b}-(K₃₆)_{b}-(K₃₇)_{b}-(K₃₈)_{b}-(K₃₉)_{b}-(K₄₀)_{b}-(K₄₁)_{b}-(K₄₂)_{b}-(K₄₃)_{b}-(K₄₄)_{b}-(K₄₅)_{b}-(K₄₆)_{b}-(K₄₇)_{b}-(K₄₈)_{b}-(K₄₉)_{b}-(K₅₀)_{b}-(K₅₁)_{b}-(K₅₂)_{b}-(K₅₃)_{b}-(K₅₄)_{b}-(K₅₅)_{b}-(K₅₆)_{b}-(K₅₇)_{b}-(K₅₈)_{b}-(K₅₉)_{b}-(K₆₀)_{b}-(K₆₁)_{b}-(K₆₂)_{b}-(K₆₃)_{b}-(K₆₄)_{b}-(K₆₅)_{b}-(K₆₆)_{b}-(K₆₇)_{b}-(K₆₈)_{b}-(K₆₉)_{b}-(K₇₀)_{b}-(K₇₁)_{b}-(K₇₂)_{b}-(K₇₃)_{b}-(K₇₄)_{b}-(K₇₅)_{b}-(K₇₆)_{b}-(K₇₇)_{b}-(K₇₈)_{b}-(K₇₉)_{b}-(K₈₀)_{b}-(K₈₁)_{b}-(K₈₂)_{b}-(K₈₃)_{b}-(K₈₄)_{b}-(K₈₅)_{b}-(K₈₆)_{b}-(K₈₇)_{b}-(K₈₈)_{b}-K₈₉-K₈₉-K₈₉-K₈₉-K₈₉ (Formula XI)

wherein:
each b is, independently, 0, 1, 2, or 3;
   wherein:
each K₁ is, independently, an amino acid selected from the group consisting of S, G, D, A, C, P, and Y;
each K₂ is, independently, an amino acid selected from the group consisting of Q, S, E, T, R, K, G, A, Y, M, V, and I;
each K₃ is, independently, an amino acid selected from the group consisting of G, S, N, T, Q, D, P, L, F, V, K, A, and C;
each K₄ is, independently, an amino acid selected from the group consisting of R, G, N, D, A, P, Y, and L;
each K₅ is, independently, an amino acid selected from the group consisting of E, A, V, Q, G, Y, M, I, and L;
each K₆ is, independently, an amino acid selected from the group consisting of S, Q, R, T, D, G, E, A, and K;
each K₇ is, independently, an amino acid selected from the group consisting of N, Q, R, H, K, A, I, F, and L;
each K₈ is, independently, an amino acid selected from the group consisting of A, T, Q, G, R, K, D, L, F, C, V, S, and H;
each K₉ is, independently, an amino acid selected from the group consisting of G, S, N, T, Q, D, P, L, F, V, K, A, and C;
each K₁₀ is, independently, an amino acid selected from the group consisting of K, H, E, A, Y, L, and F;
each K₁₁ is, independently, an amino acid selected from the group consisting of S, T, K, E, A, C, W, F, and L;
each K₁₂ is, independently, an amino acid selected from the group consisting of K, R, H, S, Q, D, E, and A;
each K₁₃ is, independently, an amino acid selected from the group consisting of G, S, T, E, P, W, R, N, and Q;
each K₁₄ is, independently, an amino acid selected from the group consisting of D, Q, S, G, V, E, N, H, R, P, and F;
each K₁₅ is, independently, an amino acid selected from the group consisting of C, A, M, V, S, E, G, I, F, and L;
each K₁₆ is, independently, an amino acid selected from the group consisting of R, K, S, Q, T, Y, N, V, I, L, and C;
each K₁₇ is, independently, an amino acid selected from the group consisting of A, G, S, Q, Y, E, D, H, and I;
each K₁₈ is, independently, an amino acid selected from the group consisting of R, K, S, Q, T, Y, N, V, I, L, and C;
each K₁₉ is, independently, an amino acid selected from the group consisting of E, D, T, H, K, G, P, V, and L;
each K₂₀ is, independently, an amino acid selected from the group consisting of F, L, I, V, M, T, G, and R;
each K₂₁ is, independently, an amino acid selected from the group consisting of E, D, S, G, A, C, and P;
each K₂₂ is, independently, an amino acid selected from the group consisting of D, T, G, A, Y, N, S, C, P, W, and I;
each K₂₃ is, independently, an amino acid selected from the group consisting of G, S, N, E, D, Y, and L;
each K₂₄ is, independently, an amino acid selected from the group consisting of T, S, E, G, P, and I;
each K₂₅ is, independently, an amino acid selected from the group consisting of K, S, G, T, and L;
each K₂₆ is, independently, an amino acid selected from the group consisting of S, G, K, E, D, P, and F;
each K₂₇ is, independently, an amino acid selected from the group consisting of P, A, E, L, T, Q, S, G, K, Y, F, C, V, W, and R;
each K₂₈ is, independently, an amino acid selected from the group consisting of E, D, Q, S, T, P, and L;
each K₂₉ is, independently, an amino acid selected from the group consisting of A, T, S, E, V, W, and I;
each K₃₀ is, independently, an amino acid selected from the group consisting of K, H, S, G, N, Q, P, and Y;
each K₃₁ is, independently, an amino acid selected from the group consisting of L, F, V, P, A, N, G, and H;
each K₃₂ is, independently, an amino acid selected from the group consisting of A, G, N, P, R, E, and K;
each K₃₃ is, independently, an amino acid selected from the group consisting of R, S, N, A, P, Y, V, I, F, and G;
each K₃₄ is, independently, an amino acid selected from the group consisting of E, S, T, V, I, H, A, P, F, and L;
each K₃₅ is, independently, an amino acid selected from the group consisting of A, T, Q, P, R, V, N, E, and L;
each K₃₆ is, independently, an amino acid selected from the group consisting of R, K, H, G, Q, D, T, Y, and F;
each K₃₇ is, independently, an amino acid selected from the group consisting of D, E, N, T, C, Y, V, I, and L;
each K₃₈ is, independently, an amino acid selected from the group consisting of S, Q, R, T, D, G, E, A, and K;
each K₃₉ is, independently, an amino acid selected from the group consisting of K, S, G, Q, D, E, A, M, I, and L;
each K₄₀ is, independently, an amino acid selected from the group consisting of H, K, S, D, E, T, P, and L;
each K₄₁ is, independently, an amino acid selected from the group consisting of A, T, S, N, P, V, L, and F;
each K₄₂ is, independently, an amino acid selected from the group consisting of K, D, M, V, I, L, and F;
each K₄₃ is, independently, an amino acid selected from the group consisting of G, S, N, T, Q, D, P, L, F, V, K, A, and C;
each K₄₄ is, independently, an amino acid selected from the group consisting of L, T, F, V, P, A, K, and I;
each K₄₅ is, independently, an amino acid selected from the group consisting of G, S, K, N, T, Q, D, A, P, L, F, and V;
each K₄₆ is, independently, an amino acid selected from the group consisting of L, F, Q, S, G, and D;
each K₄₇ is, independently, an amino acid selected from the group consisting of S, R, E, A, P, V, W, and L;
each K₄₈ is, independently, an amino acid selected from the group consisting of A, S, V, G, Q, R, E, D, L, T, K, F, C, and H;
each K₄₉ is, independently, an amino acid selected from the group consisting of E, S, T, R, G, A, P, and L;
each K₅₀ is, independently, an amino acid selected from the group consisting of S, N, R, A, P, and Y;
each K₅₁ is, independently, an amino acid selected from the group consisting of G, A, T, H, M, V, L, and F;
each K₅₂ is, independently, an amino acid selected from the group consisting of S, T, H, A, C, M, and L;
each K₅₃ is, independently, an amino acid selected from the group consisting of G, S, T, E, P, W, R, N, and Q;
each K₅₄ is, independently, an amino acid selected from the group consisting of S, H, Y, F, N, Q, R, T, G, and K;
each K₅₅ is, independently, an amino acid selected from the group consisting of A, T, Q, E, M, V, I, L, and F;
each K₅₆ is, independently, an amino acid selected from the group consisting of S, N, E, A, P, F, and L;
each K₅₇ is, independently, an amino acid selected from the group consisting of D, S, R, K, A, V, W, I, and F;
each K₅₈ is, independently, an amino acid selected from the group consisting of K, S, G, D, T, L, R, E, Y, and N;
each K₅₉ is, independently, an amino acid selected from the group consisting of S, R, G, A, V, and F;
each K₆₀ is, independently, an amino acid selected from the group consisting of A, T, Q, G, R, K, D, L, F, C, V, S, and H;
each K₆₁ is, independently, an amino acid selected from the group consisting of R, S, G, N, E, T, A, and V;
each K₆₂ is, independently, an amino acid selected from the group consisting of E, S, T, V, I, H, A, P, F, and L;
each K₆₃ is, independently, an amino acid selected from the group consisting of A, G, S, Q, R, E, D, V, L, T, K, F, C, and H;
each K₆₄ is, independently, an amino acid selected from the group consisting of E, A, V, Q, G, Y, M, I, and L;
each K₆₅ is, independently, an amino acid selected from the group consisting of G, S, T, E, P, W, R, N, and Q;
each K₆₆ is, independently, an amino acid selected from the group consisting of A, G, P, M, N, V, and S;
each K₆₇ is, independently, an amino acid selected from the group consisting of T, Q, E, N, S, A, Y, V, W, and F;
each K₆₈ is, independently, an amino acid selected from the group consisting of I, V, P, and A;
each K₆₉ is, independently, an amino acid selected from the group consisting of D, Q, S, G, V, E, N, H, R, P, and F;
each K₇₀ is, independently, an amino acid selected from the group consisting of G, S, R, N, T, Y, L, and F;
each K₇₁ is, independently, an amino acid selected from the group consisting of E, D, N, S, T, H, and Y;
each K₇₂ is, independently, an amino acid selected from the group consisting of L, I, W, V, A, T, S, E, R, and K;
each K₇₃ is, independently, an amino acid selected from the group consisting of G, S, K, A, C, F, N, T, Q, D, P, L, and V;
each K₇₄ is, independently, an amino acid selected from the group consisting of A, S, N, P, K, V, I, and L;
each K₇₅ is, independently, an amino acid selected from the group consisting of P, A, E, L, T, Q, S, G, K, Y, F, C, V, W, and R;
each K₇₆ is, independently, an amino acid selected from the group consisting of L, T, F, V, P, A, K, and I;
each K₇₇ is, independently, an amino acid selected from the group consisting of M, V, Y, L, A, N, E, and H;
each K₇₈ is, independently, an amino acid selected from the group consisting of D, T, G, A, Y, N, S, C, P, W, and I;
each K₇₉ is, independently, an amino acid selected from the group consisting of A, S, V, G, Q, R, E, D, L, T, K, F, C, and H;
each K₈₀ is, independently, an amino acid selected from the group consisting of K, R, S, A, P, V, I, and L;
each K₈₁ is, independently, an amino acid selected from the group consisting of F, L, V, A, T, S, E, D, R, and K;
each K₈₂ is, independently, an amino acid selected from the group consisting of L, F, M, A, N, G, and E;
each K₈₃ is, independently, an amino acid selected from the group consisting of D, S, H, A, V, I, F, and L;
each K₈₄ is, independently, an amino acid selected from the group consisting of A, T, Q, S, R, V, L, G, H, F, K, D, and C;
each K₈₅ is, independently, an amino acid selected from the group consisting of T, Q, E, N, S, A, Y, V, W, and F;
each K₈₆ is, independently, an amino acid selected from the group consisting of A, P, R, Y, K, D, M, L, and F;
each K₈₇ is, independently, an amino acid selected from the group consisting of N, S, D, T, A, P, and L;
each K₈₈ is, independently, an amino acid selected from the group consisting of R, S, N, A, P, Y, V, I, F, and G;
K₈₉ is an amino acid selected from the group consisting of K, R, H, G, E, T, Y, and I;
K₉₀ is an amino acid selected from the group consisting of R, S, G, N, Q, A, Y, and W;
K₉₁ is an amino acid selected from the group consisting of V, I, and F;
K₉₂ is an amino acid selected from the group consisting of A, G, P, M, N, V, and S; and
K₉₃ is an amino acid selected from the group consisting of E, D, Q, S, R, K, M, and L
   wherein Formula XIV is given by:

(M₁)_{b}-(M₂)_{b}-(M₃)_{b}-(M₄)_{b}-(M₅)_{b}-(M₆)_{b}-(M₇)_{b}-(M₈)_{b}-(M₉)_{b}-(M₁₀)_{b}-(M₁₁)_{b}- (M₁₂)_{b}-(M₁₃)_{b}-(M₁₄)_{b}-(M₁₅)_{b}-(M₁₆)_{b}-(M₁₇)_{b}-(M₁₈)_{b}-(M₁₉)_{b}-(M₂₀)_{b}-(M₂₁)_{b}-(M₂₂)_{b}-(M₂₃)_{b}-(M₂₄)_{b}-(M₂₅)_{b}-(M₂₆)_{b}-(M₂₇)_{b}-(M₂₈)_{b}-(M₂₉)_{b}-(M₃₀)_{b}-(M₃₁)_{b}-(M₃₂)_{b}-(M₃₃)_{b}-(M₃₄)_{b}-(M₃₅)_{b}-(M₃₆)_{b}-(M₃₇)_{b}-(M₃₈)_{b}-(M₃₉)_{b}-(M₄₀)_{b}-(M₄₁)_{b}-(M₄₂)_{b}-(M₄₃)_{b}-(M₄₄)_{b}-(M₄₅)_{b}-(M₄₆)_{b}-(M₄₇)_{b}-(M₄₈)_{b}-(M₄₉)_{b}-(M₅₀)_{b}-(M₅₁)_{b}-(M₅₂)_{b}-(M₅₃)_{b}-(M₅₄)_{b}-(M₅₅)_{b}-(M₅₆)_{b}-(M₅₇)_{b}-(M₅₈)_{b}-(M₅₉)_{b}-(M₆₀)_{b}-(M₆₁)_{b}-(M₆₂)_{b}-(M₆₃)_{b}-(M₆₄)_{b}-(M₆₅)_{b}-(M₆₆)_{b}-(M₆₇)_{c}-(M₆₈)_{c}-(M₆₉)_{c}-(M₇₀)_{c} (Formula XIV);

wherein:
each b is, independently, 0, 1, 2, or 3; and
each c is, independently, 1 or 2;
   wherein:
each M₁ is, independently, an amino acid selected from the group consisting of A, T, C, S, Y, E, H, V, W, I, L, F, G, Q, N, P, R, K, D, and M;
each M₂ is, independently, an amino acid selected from the group consisting of S, T, A, N, R, G, E, P, V, F, L, Q, K, H, D, I, C, Y, M, and W;
each M₃ is, independently, an amino acid selected from the group consisting of G, S, R, A, T, Q, E, D, C, Y, V, I, L, and N;
each M₄ is, independently, an amino acid selected from the group consisting of R, H, N, Q, E, A, Y, M, V, W, F, and L;
each M₅ is, independently, an amino acid selected from the group consisting of P, Y, A, T, Q, S, G, D, R, K, C, V, I, L, and H;
each M₆ is, independently, an amino acid selected from the group consisting of T, Q, N, S, A, E, G, D, H, P, F, L, C, K, V, R, Y, I, M, and W;
each M₇ is, independently, an amino acid selected from the group consisting of A, G, S, Q, N, K, D, T, C, Y, E, H, V, W, I, L, F, P, R, and M;
each M₈ is, independently, an amino acid selected from the group consisting of T, Q, N, S, A, G, C, R, K, P, Y, M, V, I, L, F, E, W, D, and H;
each M₉ is, independently, an amino acid selected from the group consisting of G, S, H, P, R, A, T, Q, E, D, C, Y, V, I, L, N, W, F, K, and M;
each M₁₀ is, independently, an amino acid selected from the group consisting of Q, E, and W;
each M₁₁ is, independently, an amino acid selected from the group consisting of V, I, L, F, C, A, and T;
each M₁₂ is, independently, an amino acid selected from the group consisting of S, G, A, N, Q, R, T, K, E, H, D, P, I, F, V, C, Y, L, M, and W;
each M₁₃ is, independently, an amino acid selected from the group consisting of T, Q, N, S, D, P, F, A, E, G, H, L, C, K, V, R, Y, I, M, and W;
each M₁₄ is, independently, an amino acid selected from the group consisting of L, F, I, V, M, Y, A, T, Q, N, S, D, K, P, E, R, H, G, and C;
each M₁₅ is, independently, an amino acid selected from the group consisting of S, P, V, E, T, A, F, L, N, R, G, Q, K, H, D, I, C, Y, M, and W;
each M₁₆ is, independently, an amino acid selected from the group consisting of T, S, A, E, G, C, R, P, Y, M, V, W, I, F, L, Q, N, D, H, and K;
each M₁₇ is, independently, an amino acid selected from the group consisting of D, E, Q, T, K, P, F, N, S, G, A, Y, R, and V;
each M₁₈ is, independently, an amino acid selected from the group consisting of G, S, H, P, R, D, N, A, T, Q, E, C, Y, V, I, L, W, F, K, and M;
each M₁₉ is, independently, an amino acid selected from the group consisting of T, P, F, S, A, E, G, C, R, Y, M, V, W, I, L, Q, N, D, H, and K;
each M₂₀ is, independently, an amino acid selected from the group consisting of L, F, I, V, Y, A, T, Q, S, D, M, N, K, P, E, R, H, G, and C;
each M₂₁ is, independently, an amino acid selected from the group consisting of F, L, W, Y, and P;
each M₂₂ is, independently, an amino acid selected from the group consisting of P, K, Y, A, T, Q, S, G, D, R, C, V, I, L, and H;
each M₂₃ is, independently, an amino acid selected from the group consisting of T, P, F, S, A, E, G, C, R, Y, M, V, W, I, L, Q, N, D, H, and K
each M₂₄ is, independently, an amino acid selected from the group consisting of S, T, A, N, R, G, E, P, V, F, L, Q, K, H, D, I, C, Y, M, and W;
each M₂₅ is, independently, an amino acid selected from the group consisting of F, W, Y, and P;
each M₂₆ is, independently, an amino acid selected from the group consisting of T, P, F, Q, N, S, A, E, G, D, K, Y, C, V, I, L, and H;
each M₂₇ is, independently, an amino acid selected from the group consisting of D, E, Q, N, S, T, R, K, G, A, Y, P, V, and F;
each M₂₈ is, independently, an amino acid selected from the group consisting of T, Q, N, S, A, G, C, R, K, P, Y, M, V, I, L, F, E, W, D, and H;
each M₂₉ is, independently, an amino acid selected from the group consisting of S, T, E, A, P, V, F, L, N, R, G, Q, K, H, D, I, C, Y, M, and W;
each M₃₀ is, independently, an amino acid selected from the group consisting of D, Q, N, H, K, G, C, and Y;
each M₃₁ is, independently, an amino acid selected from the group consisting of F, L, W, Y, and P;
each M₃₂ is, independently, an amino acid selected from the group consisting of S, T, E, A, P, V, F, L, N, R, G, Q, K, H, D, I, C, Y, M, and W;
each M₃₃ is, independently, an amino acid selected from the group consisting of A, G, S, Q, N, K, D, T, C, Y, E, H, V, W, I, L, F, P, R, and M;
each M₃₄ is, independently, an amino acid selected from the group consisting of T, A, V, I, P, F, Q, N, S, E, G, D, K, Y, C, L, and H;
each M₃₅ is, independently, an amino acid selected from the group consisting of G, S, R, N, H, D, P, A, T, Q, E, C, Y, V, I, L, W, F, K, and M;
each M₃₆ is, independently, an amino acid selected from the group consisting of T, Q, S, A, E, D, K, H, P, Y, V, W, I, F, L, N, G, and C;
each M₃₇ is, independently, an amino acid selected from the group consisting of I, L, W, V, and M;
each M₃₈ is, independently, an amino acid selected from the group consisting of A, G, S, Q, N, K, D, C, P, R, Y, E, V, W, T, H, M, and F;
each M₃₉ is, independently, an amino acid selected from the group consisting of S, T, E, P, V, A, F, L, N, R, G, Q, K, H, D, I, C, Y, M, and W;
each M₄₀ is, independently, an amino acid selected from the group consisting of T, S, A, D, P, M, Q, E, K, H, Y, V, W, I, F, L, N, G, and C;
each M₄₁ is, independently, an amino acid selected from the group consisting of L, F, I, V, Y, A, T, Q, S, D, M, N, K, P, E, R, H, G, and C;
each M₄₂ is, independently, an amino acid selected from the group consisting of P, Y, A, T, Q, S, N, W, G, I, E, D, L, K, and H;
each M₄₃ is, independently, an amino acid selected from the group consisting of S, E, P, V, T, A, F, L, N, R, G, Q, K, H, D, I, C, Y, M, and W;
each M₄₄ is, independently, an amino acid selected from the group consisting of N, Q, S, E, D, T, H, K, G, A, P, W, and F;
each M₄₅ is, independently, an amino acid selected from the group consisting of V, I, L, F, C, A, and T;
each M₄₆ is, independently, an amino acid selected from the group consisting of A, T, S, N, R, Y, K, D, H, M, L, F, G, Q, C, P, E, V, and W;
each M₄₇ is, independently, an amino acid selected from the group consisting of I, L, and V;
each M₄₈ is, independently, an amino acid selected from the group consisting of S, P, V, E, T, A, F, L, N, R, G, Q, K, H, D, I, C, Y, M, and W;
each M₄₉ is, independently, an amino acid selected from the group consisting of F, V, A, T, Q, N, S, E, G, D, and H;
each M₅₀ is, independently, an amino acid selected from the group consisting of L, F, I, V, Y, A, T, Q, S, D, M, N, K, P, E, R, H, G, and C;
each M₅₁ is, independently, an amino acid selected from the group consisting of G, S, R, H, D, P, N, A, T, Q, E, C, Y, V, I, L, W, F, K, and M;
each M₅₂ is, independently, an amino acid selected from the group consisting of T, N, S, G, C, R, H, A, D, P, M, Q, E, K, Y, V, W, I, F, and L;
each M₅₃ is, independently, an amino acid selected from the group consisting of I, L, W, V, and M;
each M₅₄ is, independently, an amino acid selected from the group consisting of P, K, Y, A, T, Q, S, G, D, R, C, V, I, L, and H;
each M₅₅ is, independently, an amino acid selected from the group consisting of D, E, Q, N, S, K, G, A, Y, P, F, T, R, and V;
each M₅₆ is, independently, an amino acid selected from the group consisting of L, F, I, V, Y, P, A, T, Q, N, S, G, E, D, K, H, M, C, and R;
each M₅₇ is, independently, an amino acid selected from the group consisting of S, P, V, E, T, A, F, L, N, R, G, Q, K, H, D, I, C, Y, M, and W;
each M₅₈ is, independently, an amino acid selected from the group consisting of P, M, V, I, L, and F;
each M₅₉ is, independently, an amino acid selected from the group consisting of N, Q, S, E, D, T, R, K, G, A, and Y;
each M₆₀ is, independently, an amino acid selected from the group consisting of G, S, H, P, R, D, N, A, T, Q, E, C, Y, V, I, L, W, F, K, and M;
each M₆₁ is, independently, an amino acid selected from the group consisting of S, P, V, T, A, R, K, E, H, C, Y, I, F, L, N, Q, G, D, M, and W;
each M₆₂ is, independently, an amino acid selected from the group consisting of P, K, A, Y, T, Q, S, G, D, R, C, V, I, L, and H;
each M₆₃ is, independently, an amino acid selected from the group consisting of A, G, S, N, E, K, D, H, M, V, W, I, L, F, T, R, Y, Q, C, and P;
each M₆₄ is, independently, an amino acid selected from the group consisting of D, E, Q, T, K, P, F, N, S, G, A, Y, R, and V;
each M₆₅ is, independently, an amino acid selected from the group consisting of L, V, F, I, Y, P, A, T, Q, N, S, G, E, D, K, H, M, C, and R;
each M₆₆ is, independently, an amino acid selected from the group consisting of S, N, R, T, G, K, E, H, D, A, P, V, C, Y, I, F, L, Q, M, and W;
each M₆₇ is, independently, an amino acid selected from the group consisting of K, R, H, S, G, N, Q, D, E, T, A, C, P, Y, M, V, W, I, L, and F;
each M₆₈ is, independently, an amino acid selected from the group consisting of R, K, H, S, G, N, Q, D, E, T, A, C, P, Y, M, V, W, I, L, and F;
each M₆₉ is, independently, an amino acid selected from the group consisting of S, A, N, Q, R, T, G, K, E, H, D, A, C, P, Y, M, V, W, I, F, and L; and
each M₇₀ is, independently, an amino acid selected from the group consisting of T, Q, N, S, A, E, G, D, C, R, K, H, P, Y, M, V, W, I, F, and L; and
   wherein Formula XV is given by:

(N₁)_{b}-(N₂)_{b}-(N₃)_{b}-(N₄)_{b}-(N₅)_{b}-(N₆)_{b}-(N₇)_{b}-(N₈)_{b}-(N₉)_{b}-(N₁₀)_{b}-(N₁₁)_{b}- (N₁₂)_{b}-(N₁₃)_{b}-(N₁₄)_{b}-(N₁₅)_{b}-(N₁₆)_{b}-(N₁₇)_{b}-(N₁₈)_{b}-(N₁₉)_{b}-(N₂₀)_{b}-(N₂₁)_{b}-(N₂₂)_{b}-(N₂₃)_{b}-(N₂₄)_{b}-(N₂₅)_{b}-(N₂₆)_{b}-(N₂₇)_{b}-(N₂₈)_{b}-(N₂₉)_{b}-(N₃₀)_{b}-(N₃₁)_{b}-(N₃₂)_{b}-(N₃₃)_{b}-(N₃₄)_{b}-(N₃₅)_{b}-(N₃₆)_{b}-(N₃₇)_{b}-(N₃₈)_{b}-(N₃₉)_{b}-(N₄₀)_{b}-(N₄₁)_{b}-(N₄₂)_{b}-(N₄₃)_{b}-(N₄₄)_{b}-(N₄₅)_{b}-(N₄₆)_{b}-(N₄₇)_{b}-(N₄₈)_{b}-(N₄₉)_{b}-(N₅₀)_{b}-(N₅₁)_{b}-(N₅₂)_{b}-(N₅₃)_{b}-(N₅₄)_{b}-(N₅₅)_{b}-(N₅₆)_{b}-(N₅₇)_{b}-(N₅₈)_{b}-(N₅₉)_{b}-(N₆₀)_{b}-(N₆₁)_{b}-(N₆₂)_{b}-(N₆₃)_{b}-(N₆₄)_{b}-(N₆₅)_{b}-(N₆₆)_{b}-(N₆₇)_{c}-(N₆₈)_{c}-(N₆₉)_{c}-(N₇₀)_{c}-(N₇₁)_{c} (Formula XV);

wherein:
each b is, independently, 0, 1, 2, or 3; and
each c is, independently, 1 or 2;
   wherein:
each N₁ is, independently, an amino acid selected from the group consisting of S, N, D, Q, R, T, G, E, H, A, P, M, V, K, Y, W, F, L, I, and C;
each N₂ is, independently, an amino acid selected from the group consisting of P, A, S, Y, V, T, G, I, E, and C;
each N₃ is, independently, an amino acid selected from the group consisting of T, S, G, D, C, A, L, N, R, P, Y, V, W, I, and F;
each N₄ is, independently, an amino acid selected from the group consisting of S, R, E, A, Q, K, N, D, T, G, H, C, P, Y, I, F, L, M, V, and W;
each N₅ is, independently, an amino acid selected from the group consisting of T, Q, N, G, C, M, S, A, E, D, Y, V, I, F, L, and W;
each N₆ is, independently, an amino acid selected from the group consisting of I, V, L, F, W, Y, A, T, S, E, D, and H;
each N₇ is, independently, an amino acid selected from the group consisting of P, V, A, S, N, G, E, L, and K;
each N₈ is, independently, an amino acid selected from the group consisting of A, G, Q, T, S, N, P, R, D, V, K, C, Y, W, I, L, and F;
each N₉ is, independently, an amino acid selected from the group consisting of F, Y, A, T, N, and R;
each N₁₀ is, independently, an amino acid selected from the group consisting of T, Q, N, R, K, M, S, E, D, H, P, V, W, I, F, and L;
each N₁₁ is, independently, an amino acid selected from the group consisting of A, G, Q, T, S, N, P, R, D, V, K, C, Y, W, I, L, and F;
each N₁₂ is, independently, an amino acid selected from the group consisting of S, N, Q, R, T, G, K, E, H, D, A, P, L, M, V, Y, W, F, I, and C;
each N₁₃ is, independently, an amino acid selected from the group consisting of L, F, I, W, V, M, Y, C, A, T, Q, N, S, G, E, D, and R;
each N₁₄ is, independently, an amino acid selected from the group consisting of V, I, L, A, T, S, G, R, P, Y, N, H, C, M, F, Q, E, K, and D;
each N₁₅ is, independently, an amino acid selected from the group consisting of S, N, Q, T, G, K, E, H, D, A, C, P, Y, I, F, L, R, M, V, and W;
each N₁₆ is, independently, an amino acid selected from the group consisting of T, N, S, A, D, R, P, Y, V, W, I, F, and L;
each N₁₇ is, independently, an amino acid selected from the group consisting of S, N, Q, R, K, E, D, A, T, G, H, C, P, Y, I, F, L, M, V, and W;
each N₁₈ is, independently, an amino acid selected from the group consisting of V, A, T, S, G, R, W, I, C, L, F, E, D, K, P, Y, N, H, M, and Q;
each N₁₉ is, independently, an amino acid selected from the group consisting of T, Q, N, S, A, E, G, D, Y, M, V, I, F, L, and W;
each N₂₀ is, independently, an amino acid selected from the group consisting of S, Q, R, K, E, A, N, D, T, G, H, C, P, Y, I, F, L, M, V, and W;
each N₂₁ is, independently, an amino acid selected from the group consisting of V, W, I, C, L, F, A, T, S, E, D, K, G, R, P, Y, N, H, M, and Q;
each N₂₂ is, independently, an amino acid selected from the group consisting of T, Q, N, S, A, D, C, K, P, Y, M, V, W, I, F, G, E, H, R, and L;
each N₂₃ is, independently, an amino acid selected from the group consisting of L, F, I, V, P, A, T, Q, S, G, R, K, H, M, Y, and D;
each N₂₄ is, independently, an amino acid selected from the group consisting of T, Q, S, A, G, P, Y, I, K, H, V, F, L, N, D, C, M, W, E, and R;
each N₂₅ is, independently, an amino acid selected from the group consisting of S, R, E, A, Q, K, N, D, T, G, H, C, P, Y, I, F, L, M, V, and W;
each N₂₆ is, independently, an amino acid selected from the group consisting of T, N, D, S, A, R, P, Y, V, W, I, F, and L;
each N₂₇ is, independently, an amino acid selected from the group consisting of D, N, R, E, Q, S, H, T, K, G, W, I, P, and Y;
each N₂₈ is, independently, an amino acid selected from the group consisting of V, A, T, S, G, R, W, I, C, L, F, E, D, K, P, Y, N, H, M, and Q;
each N₂₉ is, independently, an amino acid selected from the group consisting of T, S, A, D, C, L, N, R, P, Y, V, W, I, and F;
each N₃₀ is, independently, an amino acid selected from the group consisting of P, Y, V, A, T, S, G, I, E, and C;
each N₃₁ is, independently, an amino acid selected from the group consisting of T, Q, S, A, G, K, H, P, Y, V, I, F, L, N, D, C, M, W, E, and R;
each N₃₂ is, independently, an amino acid selected from the group consisting of S, R, E, A, Q, K, N, D, T, G, H, C, P, Y, I, F, L, M, V, and W;
each N₃₃ is, independently, an amino acid selected from the group consisting of E, D, Q, N, S, T, H, R, G, A, P, F, and L;
each N₃₄ is, independently, an amino acid selected from the group consisting of D, N, R, E, Q, S, H, T, K, G, W, I, P, and Y;
each N₃₅ is, independently, an amino acid selected from the group consisting of T, Q, S, A, G, P, Y, I, K, H, V, F, L, N, D, C, M, W, E, and R;
each N₃₆ is, independently, an amino acid selected from the group consisting of G, S, K, A, T, Q, D, C, P, Y, V, W, I, L, and F;
each N₃₇ is, independently, an amino acid selected from the group consisting of F, Y, A, T, N, and R;
each N₃₈ is, independently, an amino acid selected from the group consisting of V, A, T, S, G, R, W, I, C, L, F, E, D, K, P, Y, N, H, M and Q;
each N₃₉ is, independently, an amino acid selected from the group consisting of L, F, I, W, V, M, C, A, T, Q, N, S, G, D, R, K, and H;
each N₄₀ is, independently, an amino acid selected from the group consisting of P, A, S, Y, V, T, G, I, E, and C;
each N₄₁ is, independently, an amino acid selected from the group consisting of D, N, R, G, Y, E, Q, S, H, T, K, W, and I;
each N₄₂ is, independently, an amino acid selected from the group consisting of S, R, E, A, N, T, G, P, V, Q, K, H, D, Y, M, I, F, L, C, and W;
each N₄₃ is, independently, an amino acid selected from the group consisting of G, S, R, K, A, N, Q, H, E, D, P, W, L, and F;
each N₄₄ is, independently, an amino acid selected from the group consisting of T, Q, S, A, G, P, Y, I, N, E, D, C, K, H, R, V, L, M, F, and W;
each N₄₅ is, independently, an amino acid selected from the group consisting of S, T, G, A, V, I, R, E, N, P, Q, K, H, D, Y, M, F, L, C, and W;
each N₄₆ is, independently, C;
each N₄₇ is, independently, an amino acid selected from the group consisting of S, N, R, T, G, K, E, H, D, A, P, Y, V, W, I, L, Q, M, F, and C;
each N₄₈ is, independently, an amino acid selected from the group consisting of G, S, R, K, N, T, Q, H, E, D, P, I, and L;
each N₄₉ is, independently, an amino acid selected from the group consisting of T, S, G, D, C, A, L, N, R, P, Y, V, W, I, and F;
each N₅₀ is, independently, an amino acid selected from the group consisting of V, A, T, S, G, I, R, P, Y, L, N, H, C, M, F, Q, E, and K;
each N₅₁ is, independently, an amino acid selected from the group consisting of A, T, G, S, Q, N, R, Y, E, H, M, V, W, I, L, and F;
each N₅₂ is, independently, an amino acid selected from the group consisting of D, E, Q, N, S, T, K, A, Y, P, M, W, I, F, and L;
each N₅₃ is, independently, an amino acid selected from the group consisting of A, T, C, G, S, N, P, R, K, D, H, M, and F;
each N₅₄ is, independently, an amino acid selected from the group consisting of L, F, I, V, P, A, T, Q, S, G, R, K, H, M, Y, and D;
each N₅₅ is, independently, an amino acid selected from the group consisting of E, D, N, T, R, K, G, A, and V;
each N₅₆ is, independently, an amino acid selected from the group consisting of A, G, Q, T, S, N, P, R, D, V, W, K, C, Y, I, L, and F;
each N₅₇ is, independently, an amino acid selected from the group consisting of Y, C, N, I, F, and L;
each N₅₅ is, independently, an amino acid selected from the group consisting of S, T, G, H, A, P, Y, V, F, L, N, R, K, E, D, W, I, Q, M, and C;
each N₅₉ is, independently, an amino acid selected from the group consisting of I, V, and L;
each N₆₀ is, independently, S
each N₆₁ is, independently, an amino acid selected from the group consisting of G, S, R, K, A, N, T, Q, E, D, P, and Y;
each N₆₂ is, independently, an amino acid selected from the group consisting of I, V, L, F, W, Y, A, T, S, E, D, and H;
each N₆₃ is, independently, an amino acid selected from the group consisting of T, Q, N, G, C, M, S, A, E, D, Y, V, I, F, L, and W
each N₆₄ is, independently, an amino acid selected from the group consisting of S, N, Q, R, G, K, E, D, P, Y, W, F, T, H, A, V, L, I, M, and C;
each N₆₅ is, independently, an amino acid selected from the group consisting of A, C, G, S, Q, N, R, Y, E, K, D, H, M, V, I, and L;
each N₆₆ is, independently, an amino acid selected from the group consisting of V, I, A, T, S, G, R, P, Y, L, N, H, C, M, F, Q, E, K, and D;
each N₆₇ is, independently, an amino acid selected from the group consisting of S, N, Q, R, T, G, K, E, H, D, A, C, P, Y, M, V, W, I, F, and L;
each N₆₈ is, independently, an amino acid selected from the group consisting of K, R, H, S, G, N, Q, D, E, T, A, C, P, Y, M, V, W, I, L, and F;
each N₆₉ is, independently, an amino acid selected from the group consisting of K, R, H, S, G, N, Q, D, E, T, A, C, P, Y, M, V, W, I, L, and F;
each N₇₀ is, independently, an amino acid selected from the group consisting of D, E, Q, N, S, H, T, R, K, G, A, C, Y, P, M, V, W, I, F, and L;
each N₇₁ is, independently, an amino acid selected from the group consisting of A, T, C, G, S, Q, N, P, R, Y, E, K, D, H, M, V, W, I, L, and F.
   20. The polypeptide of any one of embodiments 16-19, wherein m is 1 and Y₁ comprises an amino acid sequence having at least 90%, 91%, 92%, 93%, 94%, 95%, 96%, 97%, 98%, 99%, or 100% identity to an amino acid sequence selected from the group consisting of SEQ ID NO. 17, 18, 19, 20, 21, 22, 23, 24, 25, 27, 29, 34, 35, 36, 37, 38, 56, 57, 58, 74, or 75.
   21. The polypeptide of any one of embodiments 16-20, wherein Z₁ is selected from the group consisting of an antiviral, insulin, an incretin, an enzyme, an enzyme inhibitor, a hormone, a cytokine, an antibody, an antimicrobial peptide, a mucosal protein, pesticide, bactericide herbicide, fungicide, nematicide, miticide, plant growth regulator, plant growth stimulator, or fertilizer), a vaccine, a diagnostic protein, a feed conversion enzyme, a flavoring, and a nutritional protein.
   22. A yeast comprising a heterologous nucleic acid molecule encoding a polypeptide having a formula of (X₁)ₙ-(Y₁)ₘ-Z₁ wherein:
X₁ is the pre-protein signal peptide of any one of embodiments 1-11,
Y₁ is the pro-protein signal peptide of any one of embodiments 12-15, and
Z₁ is a payload protein,
wherein n is 0-1 and m is 0-1,
provided that n and m are not both 0.
   23. The yeast of embodiment 22, wherein the yeast is selected from the group consisting of Kluyveromyces, Pichia, Saccharomyces, Trichoderma, and Aspergillus.
   24. The yeast of embodiment 22, wherein the yeast is a Kluyveromyces yeast and X₁ comprises an amino acid sequence selected from Formula I or SEQ ID NO. 1 and Y₁ comprising an amino acid sequence selected from Formula VI, SEQ ID NO. 20 or SEQ ID NO. 21.
   25. The yeast of embodiment 22, wherein the yeast is a Pichia yeast (e.g., P. pastoris) and X₁ comprises an amino acid sequence selected from Formula II or SEQ ID NO. 2, 3, 4, 5, 6, or 7 and Y₁ comprises an amino acid sequence selected from Formula VI, SEQ ID NO. 20 or SEQ ID NO. 21.
   26. The yeast of embodiment 22, wherein the yeast is a Saccharomyces yeast and X₁ comprises an amino acid sequence selected from Formula III, Formula IV, or Formula V, or SEQ ID NO. 8, 9, 10, 11, 12, 13, 14, 15, or 16 and Y₁ comprises an amino acid sequence selected from Formula VI, Formula VII, or Formula VIII or SEQ ID NO. 18, 19, 20, 21, 22, 23, 24, or 25.
   27. The yeast of embodiment 22, wherein the yeast is a Trichoderma yeast and X₁ comprises an amino acid sequence selected from Formula IX or SEQ ID NO. 31, 32, or 33 and Y₁ comprises an amino acid sequence selected from Formula X or Formula XI or SEQ ID NO. 34, 35, 36, 37, or 38.
   28. The yeast of embodiment 22, wherein the yeast is an Aspergillus yeast (e.g., A. niger) and X₁ comprises an amino acid sequence selected from Formula XIII, or SEQ ID NO. 70, 71, 72, or 73 and Y₁ comprises an amino acid sequence selected from Formula XIV or Formula XV or SEQ ID NO. 74 or 75.
   29. The yeast of any one of embodiments 22-28, wherein Z₁ is selected from the group consisting of an antiviral, insulin, an incretin, an enzyme, an enzyme inhibitor, a hormone, pesticide, a cytokine, an antibody, an antimicrobial peptide, a mucosal protein, bactericide herbicide, fungicide, nematicide, miticide, plant growth regulator, plant growth stimulator, or fertilizer), a vaccine, a diagnostic protein, a feed conversion enzyme, a flavoring, and a nutritional protein.
   30. A method for producing a payload protein, comprising
i) transfecting a yeast with a nucleic acid encoding the polypeptide of any one of embodiments 16-21, producing an engineered yeast; and
ii) culturing the engineered yeast in an environment effective to grow the engineered yeast, and
iii) inducing secretion of the payload protein by the engineered yeast.
   31. The method of embodiment 30, wherein inducing secretion of the payload protein comprises culturing the yeast under conditions sufficient to express the polypeptide of any one of embodiments 16-21, wherein the presence of the signal peptide induces secretion of the payload protein.
   32. The method of embodiment 30 or 31, wherein the yeast is selected from the group consisting of Kluyveromyces, Pichia, Saccharomyces, Trichoderma, and Aspergillus.
   33. The method of embodiment 30 or 31, wherein the yeast is a Kluyveromyces yeast and X₁ comprises an amino acid sequence selected from Formula I or SEQ ID NO. 1 and Y₁ comprises an amino acid sequence selected from Formula VI or SEQ ID NO. 20 or SEQ ID NO. 21.
   34. The method of embodiment 30 or 31, wherein the yeast is a Pichia yeast (e.g., P. pastoris) and X₁ comprises an amino acid sequence selected from Formula II or SEQ ID NO. 2, 3, 4, 5, 6, or 7 and Y₁ comprises an amino acid sequence selected from Formula VI or SEQ ID NO. 20 or SEQ ID NO. 21.
   35. The method of embodiment 30 or 31, wherein the yeast is a Saccharomyces yeast and XI comprises an amino acid sequence selected from Formula III, Formula IV, or Formula V, or SEQ ID NO. 8, 9, 10, 11, 12, 13, 14, 15, or 16 and Y₁ comprises an amino acid sequence selected from Formula VI, Formula VII, or Formula VIII, or SEQ ID NO. 18, 19, 20, 21, 22, 23, 24, or 25.
   36. The method of embodiment 30 or 31, wherein the yeast is a Trichoderma yeast and X₁ comprises an amino acid sequence selected from Formula IX or SEQ ID NO. 31, 32, or 33 and Y₁ comprises an amino acid sequence selected from Formula X or Formula XI, or SEQ ID NO. 34, 35, 36, 37, or 38.
   37. The method of embodiment 30 or 31, wherein the yeast is an Aspergillus yeast (e.g., A. niger) and X₁ comprises an amino acid sequence selected from Formula XIII or SEQ ID NO. 70, 71, 72, or 73 and Y₁ comprises an amino acid sequence selected from Formula XIV or Formula XV or SEQ ID NO. 74 or 75.
   38. The method of any one of embodiments 29-37, wherein the yeast is grown in culture media and the method further comprises recovering the payload protein from the culture media.
   39. The method of any of embodiments 29-38, wherein Z₁ is selected from the group consisting of an antiviral, insulin, an incretin, a cytokine, an antibody, an antimicrobial peptide, a mucosal protein, an enzyme, an enzyme inhibitor, a hormone, pesticide, bactericide herbicide, fungicide, nematicide, miticide, plant growth regulator, plant growth stimulator, fertilizer, a vaccine, a diagnostic protein, a feed conversion enzyme, a flavoring, or a nutritional protein.
   40. A method for treating a disease or a condition in a subject in need thereof comprising administering to the subject a therapeutically effective amount of the yeast of any one of embodiments 22-29.
   41. The method of embodiment 40, wherein the disease or condition is selected from an infection, an autoimmune disease, primary (congenital) enzymatic deficiency, enzymatic deficiencies secondary to functional gut disorders, diabetes, obesity, a metabolic disorder, intestinal bacterial overgrowth, enteric infection, bacterial vaginosis, inflammatory bowel disease, irritable bowel syndrome, small bowel syndrome, Celiac disease, gluten intolerance, colitis, peptic ulcer, or another GI condition or disorder.
   42. The method of embodiment 40 or 41, wherein the disease or condition is an enzyme deficiency and the payload protein is an enzyme.
   43. The method of embodiment 40 or 41, wherein the disease or condition is congenital sucrase-isomaltase deficiency and the payload protein is one or both of invertase and isomaltase.
   44. The method of embodiment 40 or 41, wherein the disease or condition is one or both of sucrose and isomaltase intolerance secondary to a functional gut disorder and the payload protein is one or both of invertase and isomaltase.
   45. The method of embodiment 40 or 41, wherein the disease or condition is one or more of gluten intolerance, refractory sprue, or Celiac disease and the payload protein is one or more of An-PEP, Mx-PEP, Aspergillus tubigensis prolyl endopeptidase, subtilisin, sedolisin, and larozotide.
   46. The method of embodiment 40 or 41, wherein the disease or condition is pancreatitis or exocrine pancreatic insufficiency and the payload protein is selected from one or more of triacylglycerol lipase, colipase, alpha-amylase, trypsin, and chymotrypsin.
   47. The method of embodiment 40 or 41, wherein the disease or condition is enteropeptidase deficiency or enterokinase deficiency and the payload protein is one or all of enteropeptidase, proenteropeptidase, and enterokinase.
   48. The method of embodiment 40 or 41, wherein the disease or condition is small intestinal bacterial overgrowth, inflammatory bowel disease, irritable bowel syndrome, C. difficile infection, cystic fibrosis, necrotizing enterocolitis, and diabetes, and the payload protein is intestinal alkaline phosphatase.
   49. The method of embodiment 40 or 41, wherein the disease or condition is short bowel syndrome and the payload protein is IGF-1, GLP-2, or a synthetic derivative of GLP-2.
   50. The method of embodiment 40 or 41, wherein the disease or condition is lactose sensitivity or lactose intolerance and the payload protein is lactase.
   51. The method of embodiment 40 or 41, wherein the disease or condition is trehalose sensitivity or lactose intolerance and the payload protein is trehalase.
   52. The method of embodiment 40 or 41, wherein the disease or condition is maltose sensitivity or lactose intolerance and the payload protein is maltase.
   53. The method of embodiment 40 or 41, wherein the disease or condition is pernicious anemia and the payload protein is intrinsic factor.
   54. The method of embodiment 40 or 41, wherein the disease or condition is bacterial overgrowth and the payload protein is lysozyme, nisin, a defensin, magainin, cateslytin, or any combination thereof.
   55. The method of embodiment 40 or 41, wherein the condition is a bacterial infection caused by one or more of E. coli, C. difficile, Vibrio cholera, Shigella, Salmonella, Cryptosporidium, or any combination thereof.
   56. The method of embodiment 40 or 41, wherein the condition is a viral infection.
   57. The method of embodiment 40 or 41, wherein the disease or condition is type 1 or type 2 diabetes mellitus and the payload protein is insulin, or an incretin.
   58. The method of embodiment 40 or 41, wherein the administering is oral or topical.
   59. The method of embodiment 40 or 41, wherein the disease or condition has an inflammatory component and the payload protein is IL-10, IL-22, TGFβ, an anti-TNFα antibody or fragment thereof, or any combination thereof.

### EXAMPLES

### Example 1: Effect of Synthetic Signal Peptide on Secretion of Maltose Binding Protein (MBP)

The functionality and secretion activity of a synthetic signal peptide comprising an amino acid sequence represented by SEQ ID NO. 1 (synKlac-v1) was measured by integrating a nucleic acid encoding synKlac-v1 into a commercially available expression system kit based on K. lactis, substituting the nucleic acid encoding synKlac-v1 for the standard pre-protein signal peptide α-MF. The nucleic acid (DNA) sequence encoding Formula I or SEQ ID NO. 1 is represented by the nucleotide SEQ ID NO. 39, which was obtained from the K. lactis genome.

Secretion of MBP using synthetic signal peptide synKlac-v1 was compared to production utilizing the standard construct comprising the alpha-mating factor from K. lactis (α-MF). To validate the hypothesis that the pro-protein signal peptide and the KR site are not necessary when synKlac-v1 is present, a control recombinant polypeptide that features α-MF without the pro-protein signal peptide and KR site motifs was produced (α-MF (No PPSP).

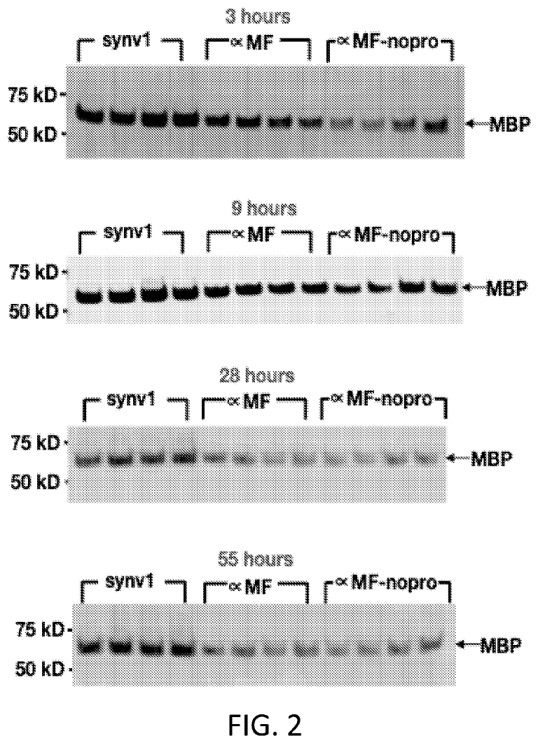

The secretion efficiency of synKlac-v1, α-MF, and α-MF (No PPSP) was assessed qualitatively and quantitatively by measuring the secretion of MBP in cell-free supernatants obtained from yeast that were grown over several intervals of time, as driven by an inducible galactose promoter. FIG. 2 shows MBP protein production detected using western blots at four different time points: 3 hours, 9 hours, 28 hours and 55 hours. Expression of MBP protein derived from each recombinant polypeptide variant was measured in four replicates using detection and quantification of a secondary antibody having an emission wavelength of 800 nm. Two samples obtained from the 3-hour time point were used to normalize the signal and allow for comparison of signal between western blot gels. Additionally, each gel featured cell-free supernatant normalized by optical density, such that protein amount detected in each lane at each time point was derived from the same number of cells, about 10⁶ colony forming units (CFUs) of K. lactis.

The results shown in FIG. 2 demonstrate that at each time point, protein secretion driven by the synKlac-v1 synthetic signal peptide outperformed protein secretion driven by α-MF, despite the lack of the pro-protein signal peptide. These results confirmed the hypothesis that the pro-protein signal peptide and the KR site are not necessary when the synthetic signal peptide synKlac-v1 is used to drive protein secretion and indicated that the synKlac-v1 function is superior across time and cell growth phase relative to secretion signal peptides currently in use. Furthermore, these results indicated that although the α-MF absent a pro-protein signal peptide remains functional, the altered α-MF has lower efficiency relative to the intact α-MF.

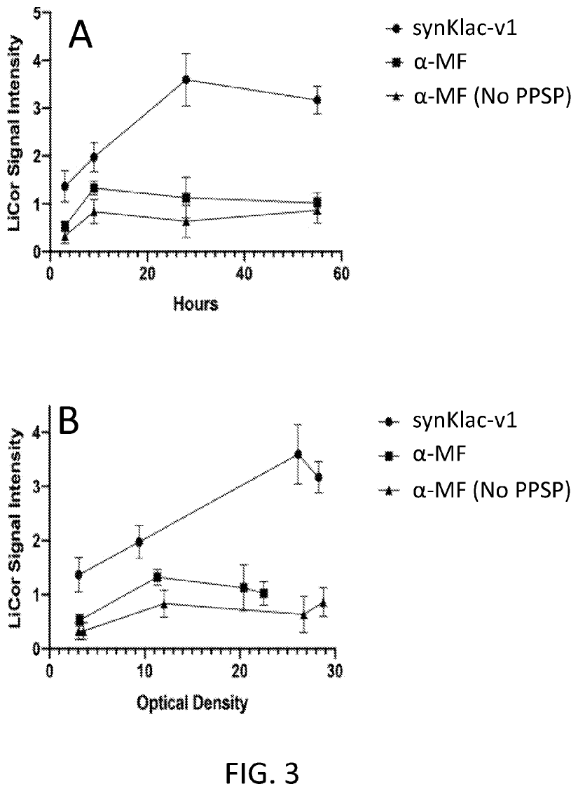

The western blot data thus obtained were quantified by measuring fluorescent signal intensity generated by the antibodies bound to MBP protein. Data for each recombinant polypeptide variant were plotted over time and cell culture growth. The results, which are shown in FIGS. 3A and 3B, indicate that even early in yeast culture, culture medium contains a higher concentration of MBP secreted using synthetic signal peptide synKlac-v1 when compared to the concentration of MBP secreted using native signal peptides α-MF or α-MF (No PPSP). The concentration of MBP protein derived from synKlac-v1 plateaus after about 25 hours of culture time (and an optical density of about 25-30), being about three times greater than the concentration of MBP secreted using native signal peptides α-MF or α-MF (No PPSP).

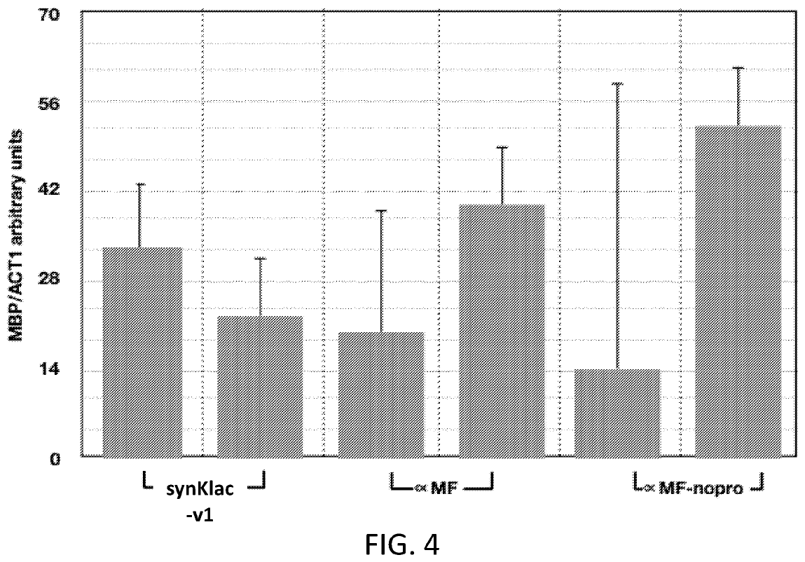

MBP transcript levels for each of the recombinant polypeptide variants were measured to confirm that the detected increases in secretion were not due to increased mRNA transcript production. FIG. 4 shows the results obtained from quantification of MBP RNA expression using quantitative PCR. RNA was collected from each sample at 28 hours, after the cell cultures were transferred to an inductive medium containing galactose. cDNA was synthesized for each sample and quantitative PCR was performed for two different yeast clones. MBP protein production was normalized to actin expression. Error bars indicate standard deviation from three biological replicate measurements for each clone. The data presented in FIG. 4 indicate that synKlac-v1 results in a higher secretion of MBP protein than α-MF in yeast, and confirmed that the significant increase in secretion is not due to increased mRNA transcript production.

### Example 2: Effect of Synthetic Signal Peptide on Secretion of TNFα

Mutations were introduced into synKlac-v1 to identify and design additional synthetic signal peptides capable of directing secretion of a payload protein from yeast. Synthetic signal pre- and pro-protein signal peptides designed according to the disclosed methods were demonstrated to increase secretion of a payload protein in all tested yeast strains, outperforming secretion driven by α-MF, which has been considered the secretion gold standard for the last 30 years.

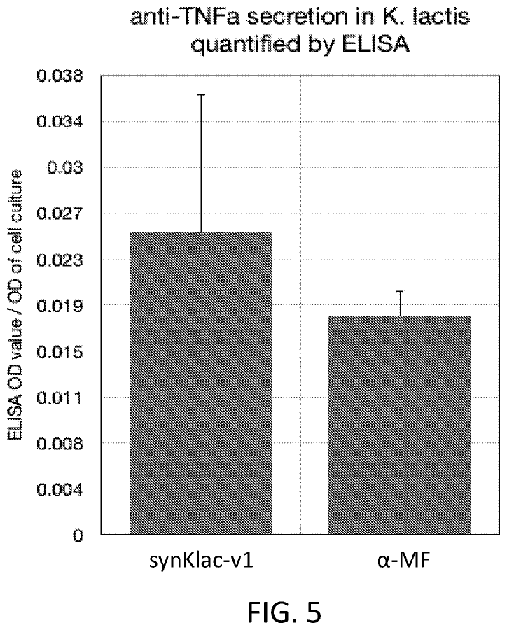

To validate the secretion efficiency of synKlac-v1 for other payloads, secretion of an anti-TNFα antibody fragment was tested. Secretion was compared between K. lactis strains which secrete anti-TNFα driven by either synKlac-v1 or α-MF. Yeast was grown in inducing medium for 24 hours after which culture supernatant was subjected to ELISA analysis. FIG. 5 depicts secretion efficiency, reported in arbitrary units derived by dividing the ELISA-derived signal values to the optical density of the cultures at 600 nm. Error bars indicate standard error of mean from four biological replicates. In summary, results in FIG. 5 indicate that synKlac-v1 induces an anti-TNFα secretion in K. lactis more than 30% greater than the secretion induced by α-MF.

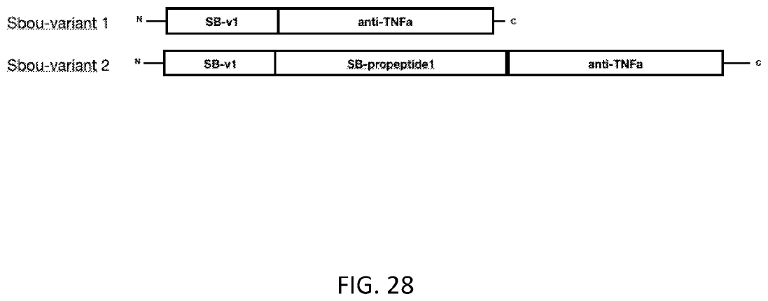

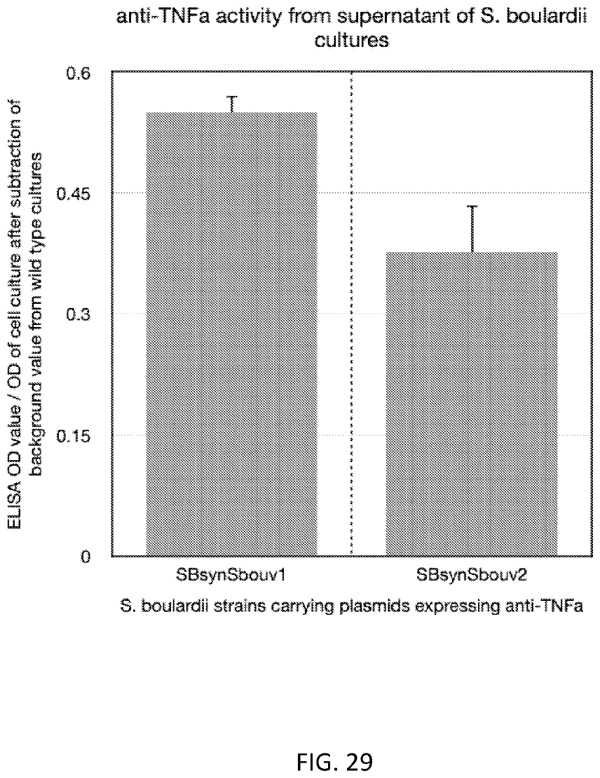

Secretion of anti-TNFα antibody fragments from S. boulardii was also investigated. Two synthetic signal peptide variants were tested, Sbou-variant 1 and Sbou-variant 2 (FIG. 28). Both variants comprise a pre-protein signal peptide as represented by SEQ ID NO. 14. Sbou-variant 1 contains no synthetic pro-protein signal peptide, while Sbou-variant 2 further comprises a pro-protein signal peptide as represented by SEQ ID NO. 22. Yeast was grown in inducing medium for 24 hours after which culture supernatant was subjected to ELISA analysis. FIG. 29 depicts secretion efficiency, reported in arbitrary units derived by dividing the ELISA-derived signal values to the optical density of the cultures at 600 nm. Error bars indicate standard error of mean from four biological replicates. In summary, results in FIG. 29 indicate that Sbou-variant 1 (no synthetic pro-protein signal peptide) has increased TNFα secretion compared to Sbou-variant 2 (which contains the pro-protein signal peptide).

### Example 3: Effect of Synthetic Signal Peptide on Secretion of Phytase

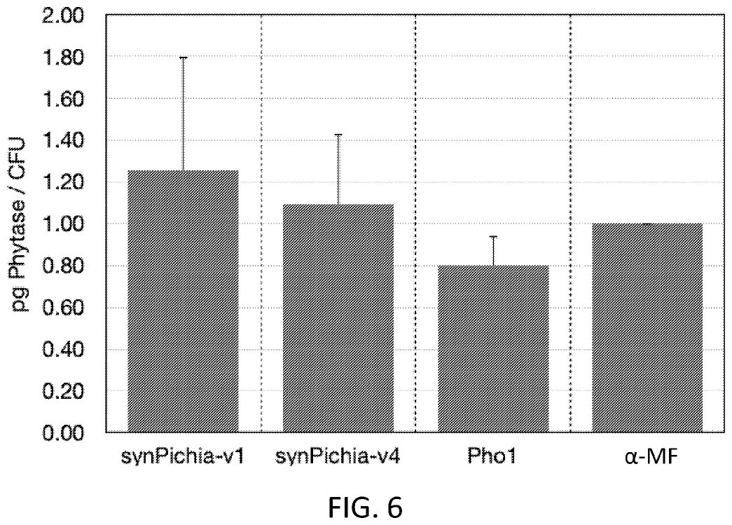

To expand the methods to other yeast strains routinely used to generate biologics or other bio-commodities, synthetic signal peptides were designed for use and expression in P. pastoris. Four recombinant polypeptide variants, each comprising a synthetic pre-protein signal peptide but lack a pro-protein signal peptide, were cloned into a commercially available expression plasmid for P. pastoris (Pichia Expression Kit—K1710-001, available from Invitrogen®.) A commercially significant version of phytase enzyme from Escherichia coli (Nov9X, ABVista®) was cloned into these plasmids to test the capability of the pre-protein signal peptide to facilitate secretion of phytase enzyme in P. pastoris against to two routinely used signal peptides in Pichia. The constructs of these recombinant polypeptide variants are depicted in FIG. 6.

The amount of phytase secreted by P. pastoris strains expressing the signal peptide from S. cerevisiae α-MF (SEQ. ID NO. 2), PHO1 (SEQ ID NO. 30), or the synthetic signal peptide comprising an amino acid sequence represented by SEQ ID NO. 4 (synPichia-v1), SEQ ID NO. 5 (synPichia-v2), SEQ ID NO. 6 (synPichia-v3) or SEQ ID NO. 7 (synPichia-v4) as measured using enzymatic activity assays. Specifically, phytase was indirectly measured through phytase activity, which was estimated by quantifying the amount of free phosphate liberated from a dodecasodium phytate substrate (7.5 mM phytate, 100 mM NaOAc, pH 5.5). Different dilutions of 50 μL P. pastoris culture supernatants (grown for 48 hours in the induction medium (BMMY)) were incubated with 100 μL of the substrate for 1 hour for at 37° C. The reaction was stopped by adding 100 μL of Color Stop reagent (ammonium molybdate, ammonium vanadate and nitric acid) and absorbance at 415 nm was measured. The amount of phytase was quantified using a standard curve generated using purified phytase enzyme from rice. The phytase amounts were then normalized to (divided by) the corresponding CFU of the culture. Normalized phytase yields for each recombinant polypeptide variant in FIG. 6 was derived from three biological replicates. The normalized phytase yield corresponding to the α-MF-phytase polypeptide was set to one (1) and the comparative yield for each other recombinant polypeptide variant is reported. Recombinant polypeptides comprising the synthetic signal peptide synPichia-v1 or synPichia-v4 exhibit up to a 20% increased secretion of phytase when compared to recombinant polypeptides comprising the native α-MF signal peptide and greater than a 40% increase when compared to a recombinant polypeptide comprising the PHO1 signal peptide. Results from recombinant polypeptides comprising synPichia-v2 and synPichia-v3 are not shown.

### Example 4: Effect of Synthetic Signal Peptide on Secretion of Insulin

To test the validity of the approach in designing a superior signal peptide and to expand the approach to the most widely used commercial yeast, S. cerevisiae, several versions of synthetic signal peptides were designed. Synthetic signal peptides contained either a synthetic pre-protein signal peptide or a synthetic pre-protein signal peptide fused with a synthetic pro-protein signal peptide. These synthetic signal peptides were cloned into a plasmid routinely used for expression of insulin in yeast and the secretion of insulin from each was measured and compared to other signal peptides routinely used in the generation of insulin from S. cerevisiae. The performance of recombinant polypeptide variants comprising the synthetic signal peptide comprising an amino acid sequence represented by SEQ ID NO. 9 fused to SEQ ID NO. 20 (synScer-v5) was compared to the performance of α-MF and the Yeast Aspartic Protease 3 (YAP) to secrete insulin.

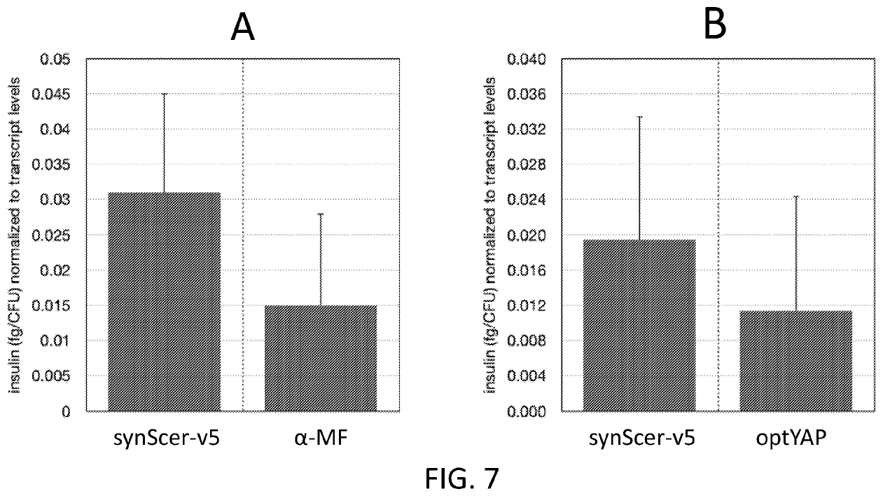

As shown in FIG. 7, insulin secretion was improved using the synthetic signal peptide. FIG. 7 shows the amount of insulin secreted per CFU in yeast strains carrying plasmids with nucleic acids encoding insulin fused to a) the synScer-v5 synthetic signal peptide, b) the α-MF signal peptide, and c) optYAP. S. cerevisiae cultures containing plasmids comprising nucleic acids encoding each recombinant polypeptide variant were grown for 48 hours and insulin in each culture supernatant was quantified using ELISA. Normalized insulin yields were generated by dividing ELISA-derived signal by the number of CFUs, which was estimated based upon the corresponding culture's optical density at 600 nm. To account for variations in transcript levels that may be due to varying plasmid copy numbers, the insulin normalized yields were normalized to the insulin mRNA levels for each variant tested. RNA was collected from each variant sample. cDNA was synthesized for each sample and quantitative PCR (qPCR) was performed. Insulin production was normalized to expression of the TAF10 (YDR167W) gene. The insulin normalized yield for each variant was then divided by the TAF10 expression value. To account for variability across different qPCR assays, the sample corresponding to the synScer-v5 variant was assayed with samples of the α-MF and optYAP variant separately. This data is presented in two separate graphs in FIGS. 7A and 7B. Error bars indicate standard error of mean from at least three biological replicate measurements. The data presented in FIG. 7 indicate that use of a synthetic signal peptide comprising a pre-protein signal peptide comprising an amino acid sequence represented by SEQ ID NO. 9 fused to SEQ ID NO. 20 provides about a 2-fold higher secretory efficiency than the α-MF and optYAP variants in S. cerevisiae.

### Example 5: Effect of Synthetic Signal Peptide on Secretion of Invertase

The different versions of an optimized signal peptide with/without synthetic pro-protein signal peptides were also tested for secretion of invertase in S. boulardii, for treatment of sucrose intolerance (e.g., congenital sucrase-isomaltase deficiency, functional gut disorders). Synthetic signal peptides contained either a synthetic pre-protein signal or a synthetic pre-protein signal fused with a synthetic pro-protein signal. Nucleic acids encoding for these synthetic signal peptides were cloned into a plasmid designed for expression of proteins in S. boulardii and the secretion of invertase from each was measured and compared to the native signal peptide present in the endogenous version of SUC2 gene which codes for the native invertase protein in S. boulardii.

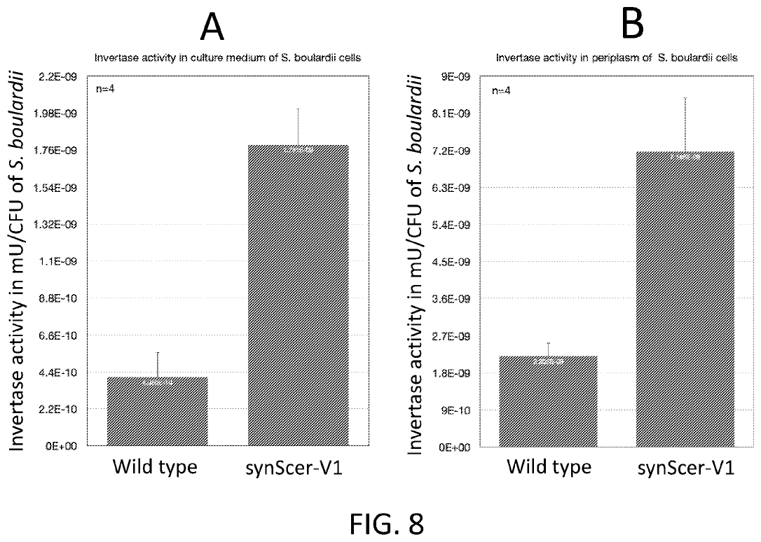

As shown in FIG. 8, invertase secretion was increased by over 150% using the synthetic signal peptide compared to using the native secretion single for invertase. FIG. 8 shows the amount of invertase secreted per CFU in yeast strains carrying plasmids with nucleic acids encoding invertase with the synthetic signal peptide comprising an amino acid represented by SEQ ID NO. 9 fused to SEQ ID NO. 25 (synScer-v1) and the native signal peptide. A majority of the secreted invertase is known to accumulate in the periplasm of yeast cells and some of it is also known to be excreted into the growth medium. S. boulardii cultures containing plasmids comprising nucleic acids encoding each recombinant polypeptide variant were grown for 24 hours in standard YPD medium with G418 antibiotic for selection of plasmids and invertase activity was assessed in culture supernatants as well as periplasmic extracts prepared by Zymolyase treatment of these cells. The recombinant invertase expressed was purified using Nickel affinity chromatography. Invertase activity was measured from purified extracts using a kit from SIGMA and normalized invertase yields were generated by dividing invertase activity by the number of CFUs, which was estimated based upon the corresponding culture's optical density at 600 nm.

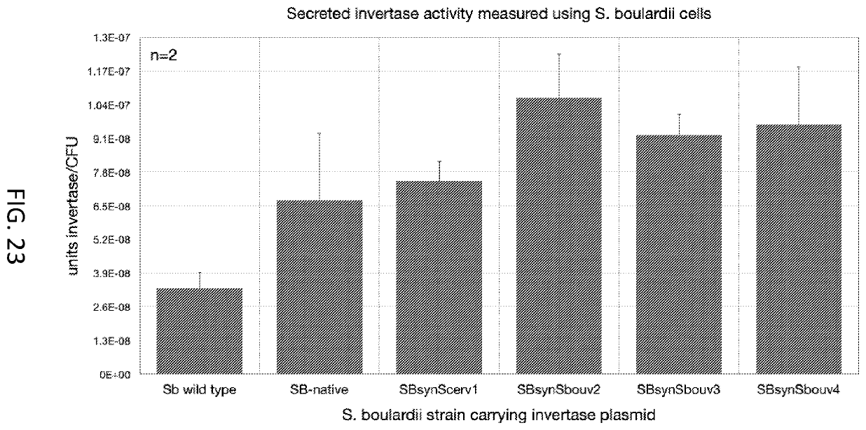

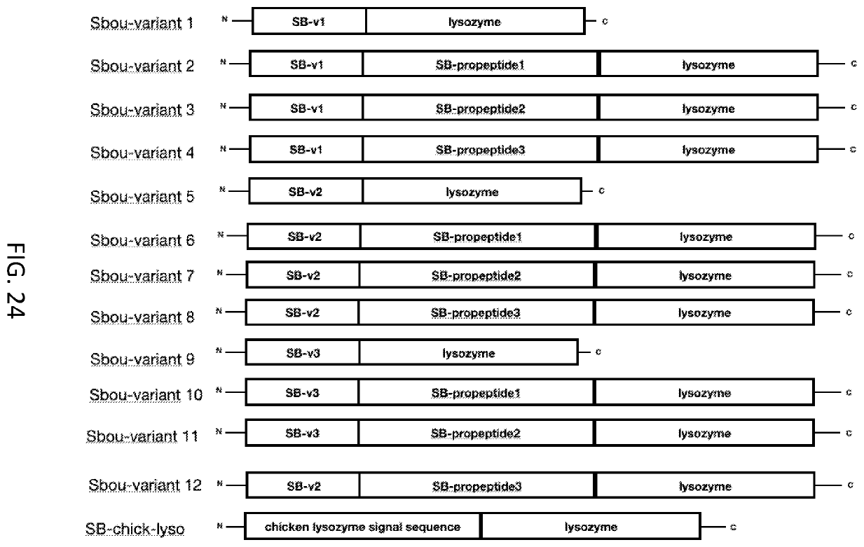

Additional synthetic peptide variants were also investigated using the same method (Sbou-variant 1-Sbou-variant 12). FIG. 22 illustrates the variants tested. The pre-protein synthetic peptide utilized by Sbou-variants 1-4 comprise an amino acid sequence represented by SEQ ID NO. 14. The pre-protein synthetic peptide utilized by Sbou-variants 5-8 comprise an amino acid sequence represented by SEQ ID NO. 15. The pre-protein synthetic peptide utilized by Sbou-variants 9-12 comprise an amino acid sequence represented by SEQ ID NO. 16. Sbou-variants 1, 5, and 9 comprise no synthetic pro-protein signal peptide. Sbou-variants 2, 6, and 10 further comprise a pro-protein synthetic signal peptide comprising an amino acid sequence represented by SEQ ID NO. 22. Sbou-variants 3, 7, and 11 further comprise a pro-protein synthetic signal peptide comprising an amino acid sequence represented by SEQ ID NO. 23. Sbou-variants 5, 8, and 12 further comprise a pro-protein synthetic signal peptide comprising an amino acid sequence represented by SEQ ID NO. 24. The results of invertase secretion from S. boulardii using these variants as compared to wild type, a native pre-protein signal peptide, and synScer-v1 is shown in FIG. 23. The results indicate that select members of the Sbou-variant class of synthetic signal peptides result in increased invertase secretion compared to wild type, native pre-protein signal peptide, and synScer-v1.

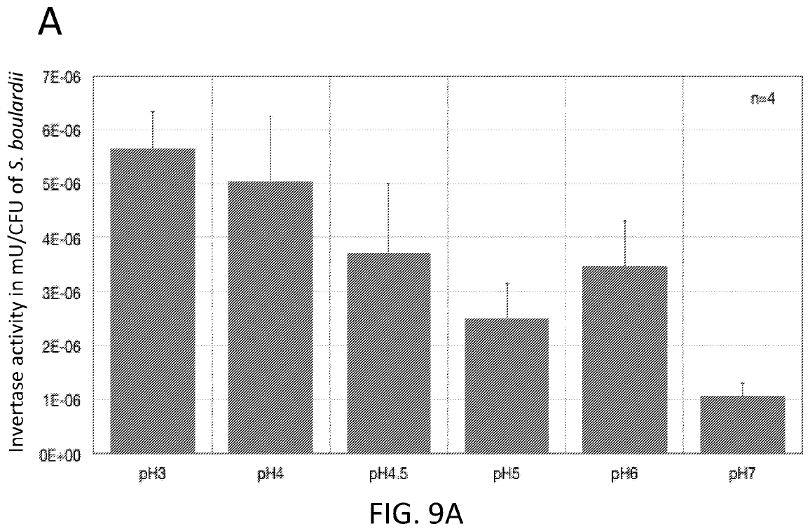

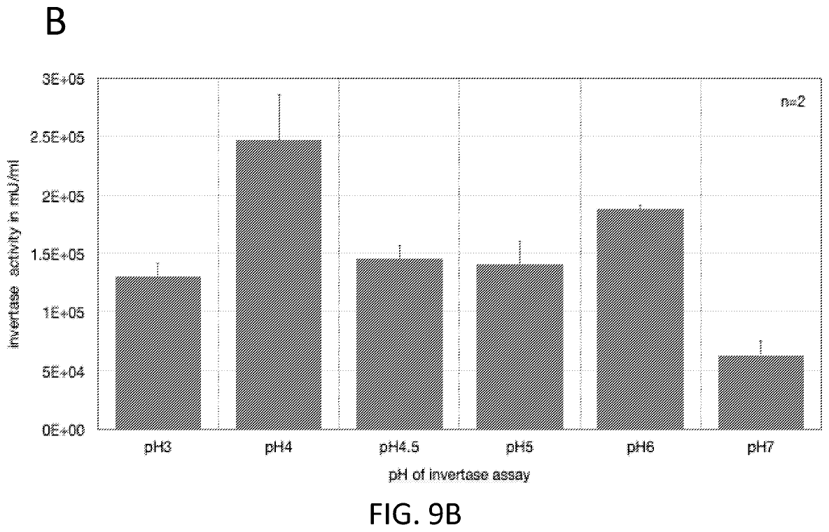

The effect of pH on the activity of the invertase enzyme secreted using the synthetic signal peptides was compared that of a pure yeast invertase enzyme obtained from SIGMA. FIG. 9 shows similar or improved activity for invertase secreted by engineered yeast as compared to the commercial, purified enzyme, thus showing that the synthetic signal peptide does not compromise the pH profile of secreted invertase.

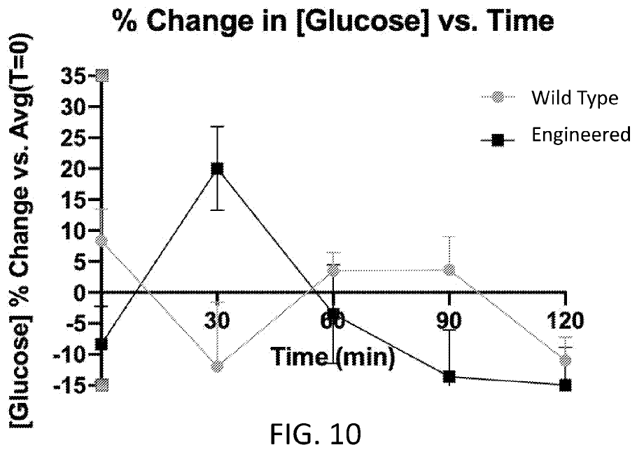

To reveal the utility of S. boulardii as a delivery agent for invertase in gut, mice were orally administered S. boulardii strains carrying plasmids encoding either invertase with the synthetic signal peptide synScer-v1 (SEQ ID NO. 9 fused to SEQ ID NO. 25) or the native signal peptide by gavage. Mice provided invertase-expressing yeast were then orally administered sucrose. Blood glucose levels were monitored as proxy for invertase activity in mice. The blood glucose levels shown in FIG. 10 indicate a higher level of invertase activity in mice provided the synScer-v1-carrying yeast, presumably due to a higher rate of secretion of the invertase by these engineered yeast.

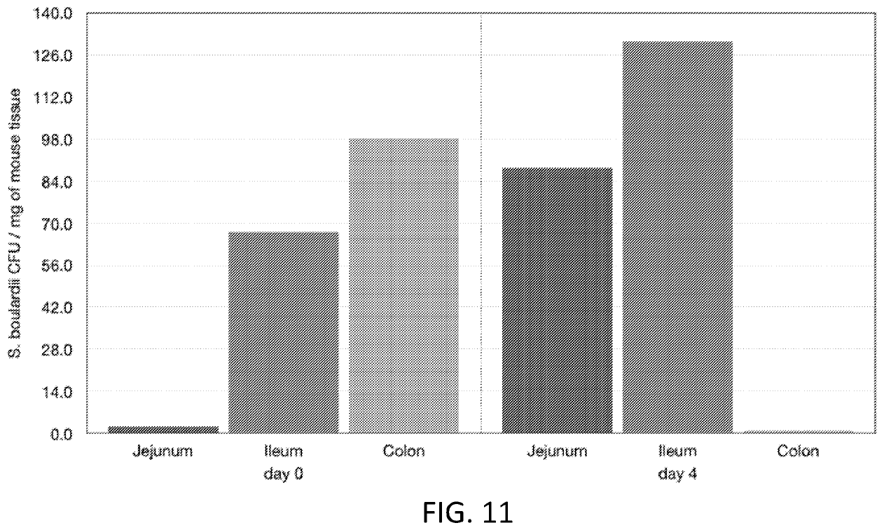

S. boulardii yeast were isolated from different tissues of the digestive system of the mice receiving the engineered yeast. Tissues were extracted from each mouse, rinsed in PBS, and then plated at different dilutions on standard growth media with G418 antibiotic. As seen in FIG. 11, mice receiving the engineered yeast seem to retain that yeast in all tissues plated. It is also noteworthy that the retention of the yeast is higher in small GI tissues in mice with colitis (treated with dextran sulfate sodium (DSS) for 4 days), thus also providing the opportunity for delivery of increased payload that may prove to be beneficial in alleviating the disease.

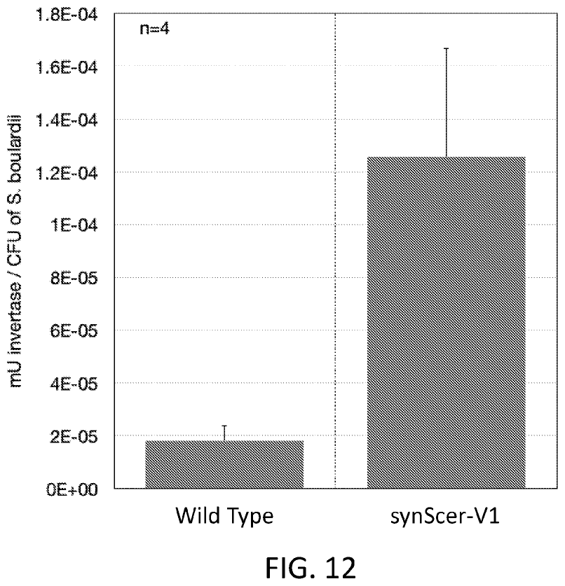

The amount of invertase secreted from S. boulardii strains carrying the plasmids encoding invertase with the synthetic signal peptide synScer-v1 were compared with S. boulardii wild type strain. The total amount per CFU was estimated by measuring the invertase activity from S. boulardii periplasmic extracts and dividing that invertase activity by the number of CFUs, which was estimated based upon the corresponding culture's optical density at 600 nm. As seen in FIG. 12, engineered S. boulardii strains produced a 7-fold higher invertase enzyme/CFU as compared to wild-type S. boulardii strains. It was estimated that about 10⁸ CFUs of the engineered S. boulardii strain are enough to produce 17,000 units of invertase activity, which is equivalent to one dose of sacrosidase (SUCRAID®), used for treatment of sucrose intolerance (e.g., congenital sucrase-isomaltase deficiency, functional gut disorders). Thus, the synthetic signal peptide used in this approach may be able to provide about a 10-fold higher sucrase payload than a corresponding dose of the wild-type S. boulardii and therefore may provide a robust delivery vehicle for delivery of important probiotic payloads. Further, SUCRAID®, which is used to treat sucrase-isomaltase disorder (CSID), includes papain which have been observed to cause allergic reactions. In contrast, the synthetic signal peptide used may be able to provide a method for treating CSID with a lower risk of allergic reaction.

### Example 6: Effect of Synthetic Signal Peptide on Secretion of IGF-1

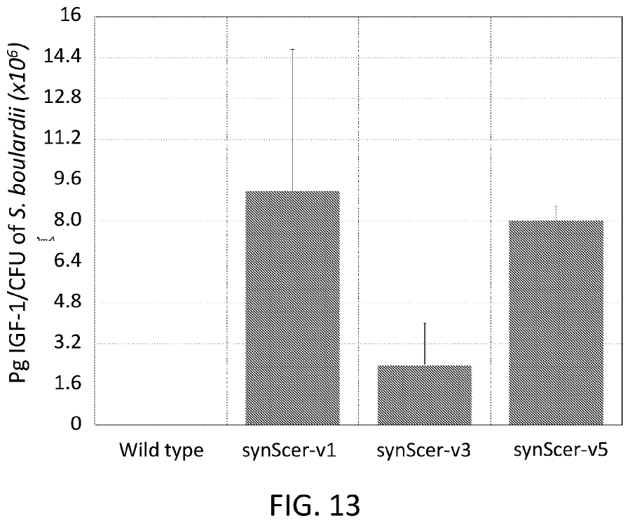

The different versions of an optimized signal peptide with synthetic signal peptides with/without synthetic pro-protein signal peptides were also tested for secretion of IGF-1 in S. boulardii, for treatment of short bowel syndrome. Specific synthetic signal peptides included SEQ ID NO. 9 combined with SEQ ID NO. 20 (synScer-v5), 21 (synScer-v4), or 25 (synScer-v1). Synthetic signal peptides contained either a synthetic pre-protein signal peptide or a synthetic pre-protein signal peptide fused with a synthetic pro-protein signal peptide. Nucleic acids encoding these synthetic signal peptides were cloned into a plasmid designed for expression of insulin-like growth factor 1 (IGF-1) protein in S. boulardii. The secretion of IGF-1 from each was measured using ELISA. Engineered and wild-type S. boulardii strains were grown for 24 hours in standard growth conditions. The level of secreted IGF-1 was quantified by performing ELISA on culture supernatants and then expressed as normalized invertase yields by dividing IGF-1 amount by the number of CFUs, which was estimated based upon the corresponding culture's optical density at 600 nm. As shown in FIG. 13, the different synthetic signal peptides exhibit robust secretion of IGF-1 in culture supernatants from S. boulardii whereas the wild-type yeast without a secretion signal do not exhibit any IGF-1 in culture supernatants.

### Example 7: Effect Signal Peptide on Secretion of Lysozyme

The different versions of an optimized signal peptide with/without synthetic pro-protein signal peptides were also tested for secretion of lysozyme in S. boulardii, for treatment of small intestinal bacterial overgrowth, pouchitis, C. difficile infection, or any other enteric infection. S. boulardii strains carrying plasmids with nucleic acids encoding lysozyme with the synthetic signal peptides or a signal peptide which is routinely used to secrete protein from yeast such as α-MF were constructed. Specific synthetic signal peptides included SEQ ID NO. 9 combined with SEQ ID NO. 20 (synScer-v5) or 21 (synScer-v4) SEQ ID NO. 9 fused to the S. cerevisiae pro-protein signal peptide α-MF (SEQ ID NO. 2).

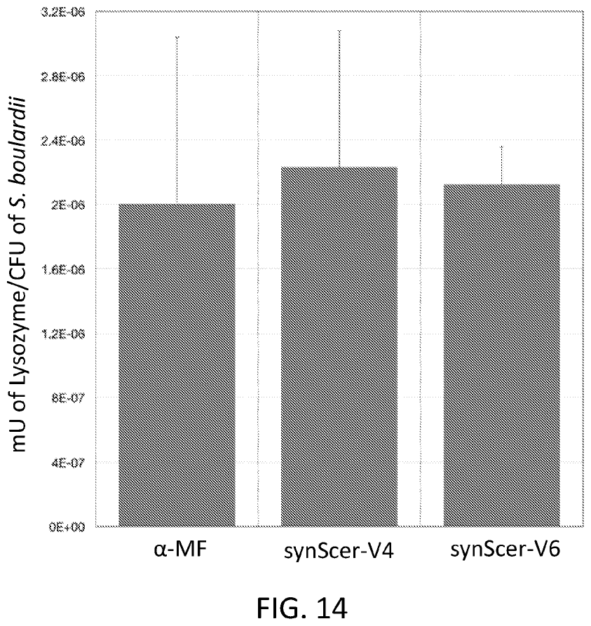

Lysozyme activity was estimated from culture supernatants of S. boulardii cultures carrying the different plasmids either encoding for α-MF signal or the synthetic signal peptides and also in swill-type S. boulardii strains without any plasmids as a control. The strain were grown for 72 hours and enzymatic activity of lysozyme was estimated using a commercially available kit. The total amount per CFU was estimated by measuring the lysozyme activity from S. boulardii supernatants, dividing lysozyme activity by the number of CFUs, which was estimated based upon the corresponding culture's optical density at 600 nm and then subtracting the background activity/CFU measured from supernatants of wild-type S. boulardii strains. As seen in FIG. 14, the strains carrying plasmids encoding the synthetic signal peptides (e.g., synScer-v4 or synScer-v5) generated ˜50% higher levels of lysozyme per CFU compared to the strain carrying the α-MF plasmid. Thus, synthetic signal peptides aid the secretion of lysozyme from S. boulardii.

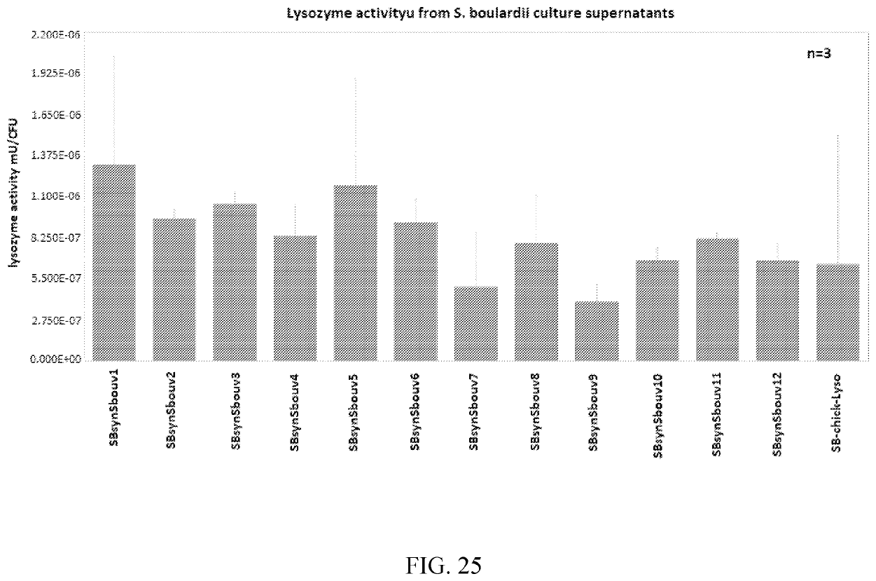

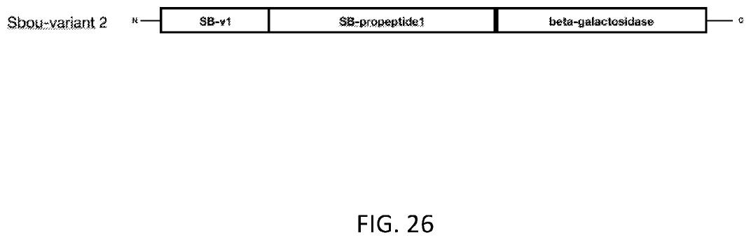

Building on these results, additional synthetic peptide variants were also investigated using the same method (Sbou-variant 1-Sbou-variant 12). FIG. 24 illustrates the variants tested. The pre-protein synthetic peptide utilized by Sbou-variants 1-4 comprise an amino acid sequence represented by SEQ ID NO. 14. The pre-protein synthetic peptide utilized by Sbou-variants 5-8 comprise an amino acid sequence represented by SEQ ID NO. 15. The pre-protein synthetic peptide utilized by Sbou-variants 9-12 comprise an amino acid sequence represented by SEQ ID NO. 16. Sbou-variants 1, 5, and 9 comprise no synthetic pro-protein signal peptide. Sbou-variants 2, 6, and 10 further comprise a pro-protein synthetic signal peptide comprising an amino acid sequence represented by SEQ ID NO. 22. Sbou-variants 3, 7, and 11 further comprise a pro-protein synthetic signal peptide comprising an amino acid sequence represented by SEQ ID NO. 23. Sbou-variants 5, 8, and 12 further comprise a pro-protein synthetic signal peptide comprising an amino acid sequence represented by SEQ ID NO. 24. An additional variant was also tested, Sbou-chickLysozyme, which comprises a pre-protein synthetic peptide signal comprising an amino acid sequence represented by SEQ ID NO. 55 and does not comprise an additional synthetic pro-protein signal peptide. The results are illustrated in FIG. 25.

As shown by FIG. 25, the efficacy of secretion greatly depended on the identities of the pre and pro-protein signal peptides. For variants comprising a pre-protein signal peptide as represented by SEQ ID NO. 14 (Sbou-variants 1-4), inclusion of a synthetic pro-protein signal peptide decreased lysozyme secretion. This observation held true for variants comprising a pre-protein signal peptide as represented by SEQ ID NO. 15 (Sbou-variants 5-8). However, for variants comprising a pre-protein signal peptide as represented by SEQ ID NO. 16 (Sbou-variants 9-12), inclusion synthetic pro-protein signal peptides increased lysozyme secretion. As such, the results indicate that there is no clear and obvious rule for increasing protein secretion (e.g. inclusion or exclusion of a pro-protein signal peptide), but rather the amount of secretion depends on the distinct identities of the pre and pro-protein signal peptides, as well as the distinct combination of individual pre and pro-protein signal peptides.

### Example 8: Biodistribution of Engineered Yeast in Mouse GI

Five, healthy, C57BL/6 mice were orally dosed with 10⁹ CFUs of S. boulardii, engineered to express a fluorescent protein (mCherry). The yeast cells were suspended in 300 mL of PBS with no other formulation excipients.

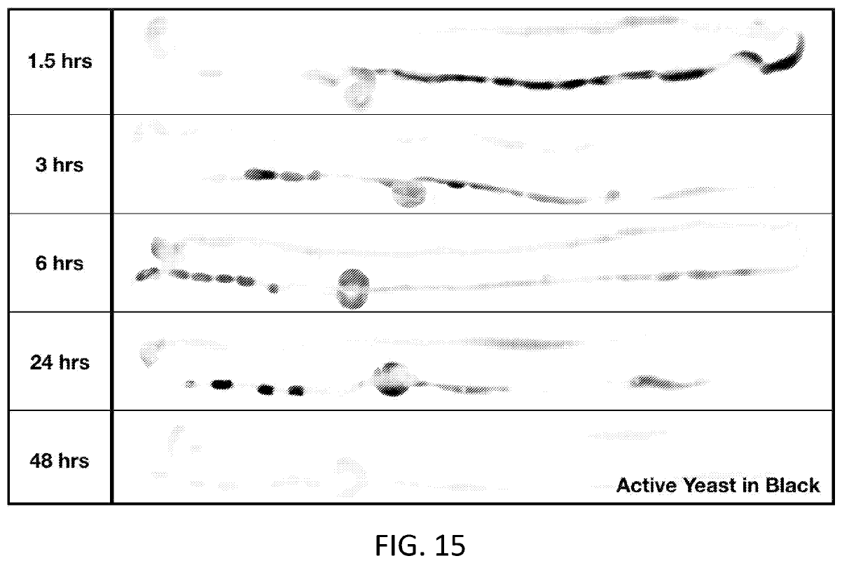

After 1.5, 3, 6, 24, and 48 hours after the oral dose, a mouse was sacrificed and its GI tract was removed imaged with a ThermoFisher, iBright CCD camera. Images are shown in FIG. 15, with fluorescent signal is reported in black.

The resulting images show the yeast survival and fluorescent protein deployment through the upper GI tract for up to 24 hours, with lower GI exposure via packaging into stool in the cecum. The yeast dose is largely depleted by 48 hours, which is consistent with previous literature indicating that S. boulardii is not a GI colonizer. This is an important property with respect to recombinant live biotherapeutics, as regulatory agencies are preferential to non-colonizing/non-engrafting chassis strains.

### Example 9: Activity after Lyophilization

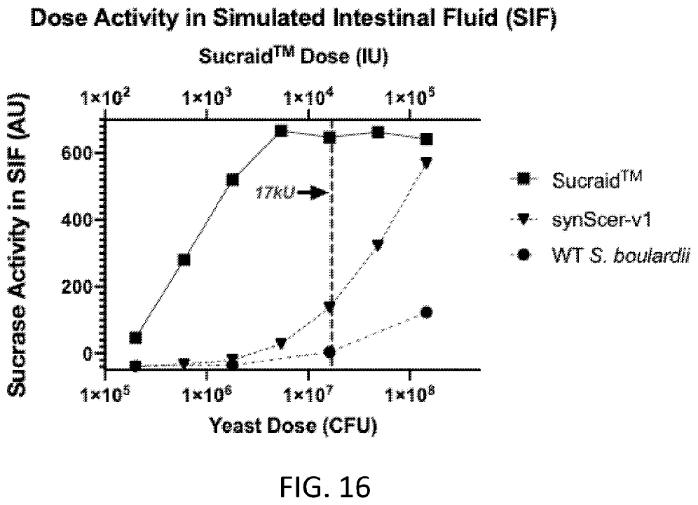

The engineered yeast, as disclosed herein, retain activity after lyophilization and freeze-drying, which is particularly advantageous given the ease of storage and transport as well as stability of this form. After lyophilization, the engineered yeast, as disclosed herein, exhibits superior activity over wild type yeast across a range of doses in conditions simulating intestinal fluid, against a physiologically representative sucrose challenge (80 mg per mL of intestinal fluid), by at least 3 fold and up to 40 fold when tested using engineered S. boulardii expressing sucrase. With such high activity, the number of CFUs needed to achieve an activity level within range of a commercially available product, e.g., SUCRAID™ (17,000 IU) from a projected 10¹⁰ CFU/dose with wild type yeast, which requires at least 1 g of lyophilized product, to 10⁸ CFU/dose with engineered yeast, which may be formulated in a dose of less than or equal to about 250 mg—a critical advantage with respect to minimizing the footprint of a pill for oral consumption, as CSID interventions must be able to be used in children from an early age. This data is presented in FIG. 16.

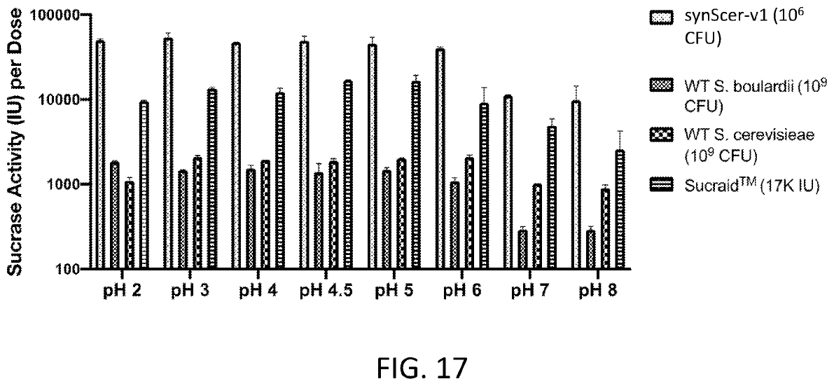

The effect of pH on the activity of the invertase enzyme secreted using the synthetic signal peptides was compared that of a pure yeast invertase enzyme obtained from SIGMA. FIG. 17 shows similar or improved activity for invertase secreted by lyophilized engineered yeast as compared to the commercial, purified enzyme, thus showing that lyophilizing the engineered yeast does not decrease change activity profile of secreted invertase at various pH compared to the non-lyophilized form, critically at pH levels below 5, which are representative of the conditions in the proximal upper gastrointestinal tract, as is shown in FIG. 9.

### Example 10: Glucose Insensitivity

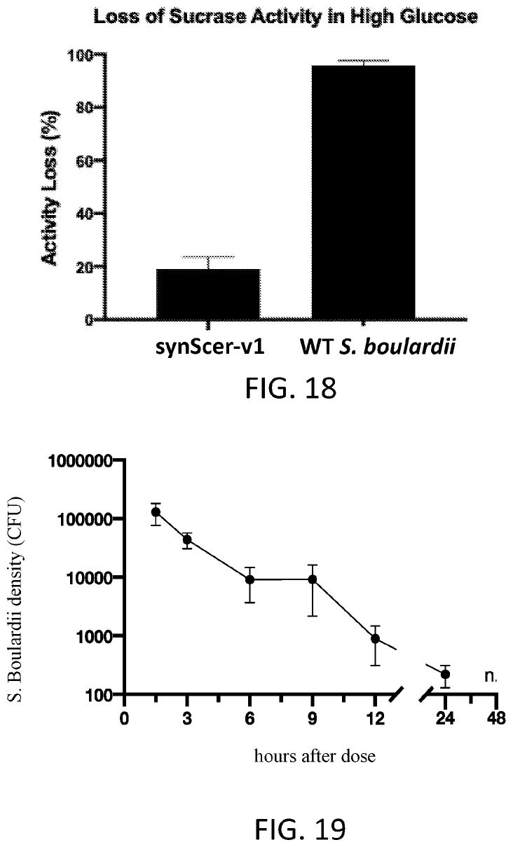

Another advantage compared to commercially available yeast products, such as SUCRAID™, is that the sucrase/invertase activity in such commercial yeast is repressed in the presence of glucose. Glucose is a byproduct of the sucrase/invertase activity itself and therefore auto-regulates the enzyme activity invertase/sucrase to lower the activity as glucose byproduct accumulates in the environment. As such, as glucose accumulates, the therapeutic activity of wild-type yeast decreases. In contrast, the engineered yeast disclosed herein utilize an expression system that is additional to the natively expressed enzyme and therefore, sucrase/invertase expression and therefore activity is insensitive to glucose. This was tested by quantifying the activity loss of our engineered S. boulardii expressing sucrase between cultures grown in 2% vs. 0.05% glucose, as compared to wild type S. boulardii. The results are shown in FIG. 18. Notably, in high glucose environments, the engineered S. boulardii yeast (left) exhibits less loss of invertase activity than the wild type S. boulardii (right).

### Example 11: Biodistribution of Invertase-Secreting Engineered Yeast in Mouse GI

Twenty-five, healthy, C57BL/6 mice were orally dosed with 10⁹ CFUs of S. boulardii, engineered to express invertase using signaling peptide synScer-v1. The yeast cells were suspended in 300 mL of PBS with no other formulation excipients.

After 1.5, 3, 6, 24, and 48 hours after the oral dose, five mice were sacrificed and its GI tract was removed and the number of CFUs of S. boulardii were quantified by homogenizing GI tissue samples resected at each time point, and plating onto petri dishes with yeast-selective agar. Results are shown in FIG. 19, where the yeast dose is persistent in the GI at the time scale of digestion and where its activity is most required (e.g., over 1-6 hours). The yeast is largely depleted by 48 hours, which is consistent with previous literature indicating that S. boulardii is not a GI colonizer. This is an important property with respect to recombinant live biotherapeutics, as regulatory agencies are preferential to non-colonizing/non-engrafting chassis strains.

### Example 12: In Vivo Activity of Invertase-Secreting Engineered Yeast in Mouse GI

Nine, healthy, freshly weened, 3 week old C57BL/6 mice were obtained and placed on a sugar-free diet for 1 week, then orally challenged with 2 g/kg of sucrose, and then orally dosed with either 10⁷ CFUs of wild type S. boulardii, S. boulardii engineered to express invertase using signaling peptide synScer-v1, or 300 μL of PBS (three mice per group). The yeast cells were suspended in 300 μL of PBS with no other formulation excipients.

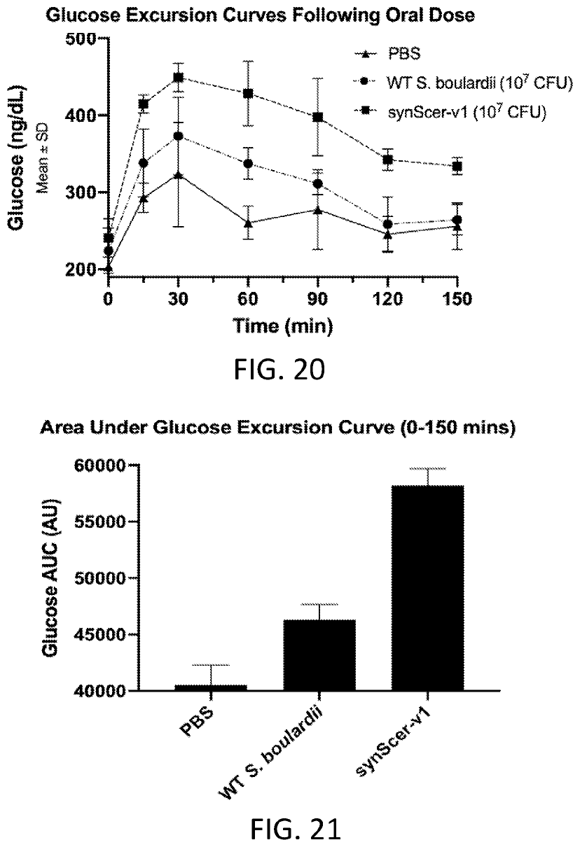

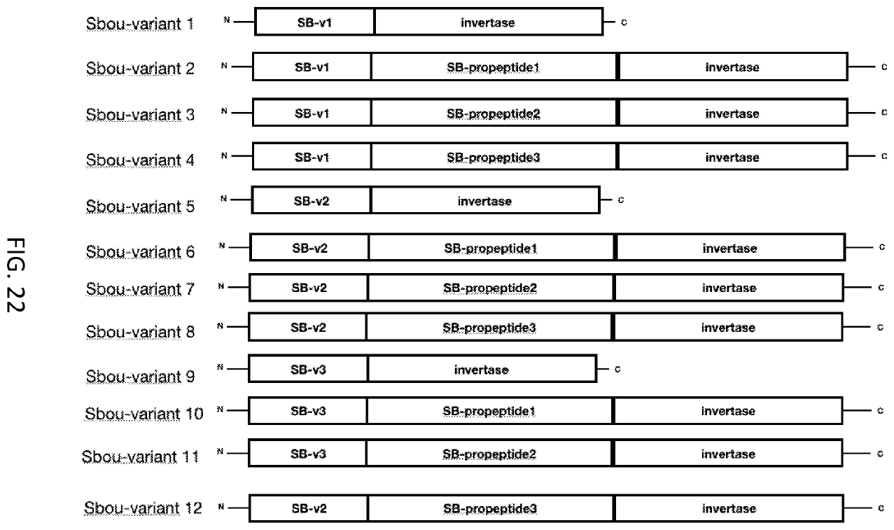

After 15, 30, 60, 90, 120 and 150 minutes after the oral dose, dose activity was measured via quantification of mouse blood glucose levels were recorded using an Accuchek™ glucometer, where an increase in blood glucose is expected as a result of breakdown of the oral sucrose challenge in the GI tract—resulting in an accumulation of glucose byproduct that is absorbed by mouse GI tissue at levels detectable in blood. Results are shown in FIG. 20, where a blood glucose excursion curves are visible with the yeast doses as compared to the PBS control, with 25% higher activity observed with engineered yeast as compared to wild-type yeast as determined by the area under their respective glucose excursion curves (AUC) as shown in FIG. 21. Critically, this result was achieved with a 10⁷ CFU dose, which is at least 10× lower than the dose anticipated to be used in humans.

### Example 13: Effect of S. boulardii Synthetic Signal Peptide on Secretion of Beta-Galactosidase or Lactase

The S. boulardii optimized signal peptide with synthetic pro-protein signal peptide was tested for secretion of lactase in S. boulardii, for treatment of lactose intolerance. Synthetic signal peptide contained a synthetic pre-protein signal used with a synthetic pro-protein signal. Nucleic acids encoding for these synthetic signal peptides were cloned into a plasmid designed for expression of proteins in S. boulardii and the secretion of lactase was measured. Wild type S. boulardii cells were used as control.

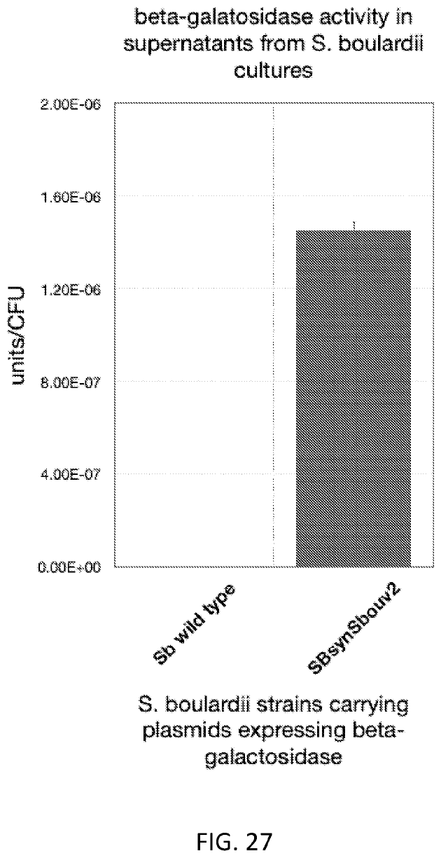

As seen in FIG. 27, S. boulardii cells have been successfully engineered to secrete lactase. FIG. 27 shows the amount of lactase secreted per CFU in yeast strains carrying plasmids with nucleic acids encoding lactase with the synthetic signal peptide comprising an amino acid represented by SEQ ID NO. 14 fused to SEQ ID NO. 22 (Sbou-variant2). The secreted lactase is excreted into the growth medium. S. boulardii cultures containing plasmids comprising nucleic acids encoding each recombinant fusion protein variant were grown for 24 hours in standard YPD medium with G418 antibiotic for selection of plasmids and lactase activity was assessed in culture supernatants. Lactase hydrolyzes lactose into glucose and galactose. The activity was measured by incubating the culture supernatants with the substrate lactose and the liberated glucose was measured using a kit from Thermo Fisher (Amplex Red Glucose assay kit, catalog number A22189), as per the manufacturer's instructions. The lactase activity was then calculated using the formula that 1 Unit of lactase activity equals amount of enzyme that generates 1.0 μmol of glucose per minute at pH 4.5 at 37° C. The amount of glucose liberated was normalized by dividing lactase activity by the number of CFUs, which was estimated based upon the corresponding culture's optical density at 600 nm. It was estimated that about 10\hat{ }9 CFUs of the engineered S. boulardii strain are enough to produce 9000 units of lactase activity, which is equivalent to one dose of LACTAID®, used for treatment of lactose intolerance. As 10\hat{ }9 CFUs of S. boulardii is an industry standard quantity of yeast that is formulated for oral probiotic products, these data indicate the viability of S. boulardii strains engineered for lactase secretion via fusion to SEQ ID NO. 14 and SEQ ID NO. 22 represent a therapeutically viable intervention for lactose intolerance.

### Example 14: Effect of S. boulardii Synthetic Signal Peptide on Secretion of Anti-TNFα Antibody Fragments

The different versions of an optimized signal peptide with/without synthetic pro-protein signal peptide were also tested for secretion of different versions of anti-TNFα antibody fragments in S. boulardii. anti-TNFα antibodies are used in clinical gold standard therapies for inflammatory diseases, including inflammatory bowel disease in the gut. A monovalent or bivalent form of anti-TNFα antibody fragments delivered by engineered yeast may similarly be used for therapeutic purposes in the gut. Synthetic signal peptides contained either a synthetic pre-protein signal or a synthetic pre-protein signal fused with a synthetic pro-protein signal. Nucleic acids encoding for these synthetic signal peptides were cloned into a plasmid designed for expression of proteins in S. boulardii and the secretion of either a monovalent or a bivalent form of anti-TNFα antibody fragments were analyzed.

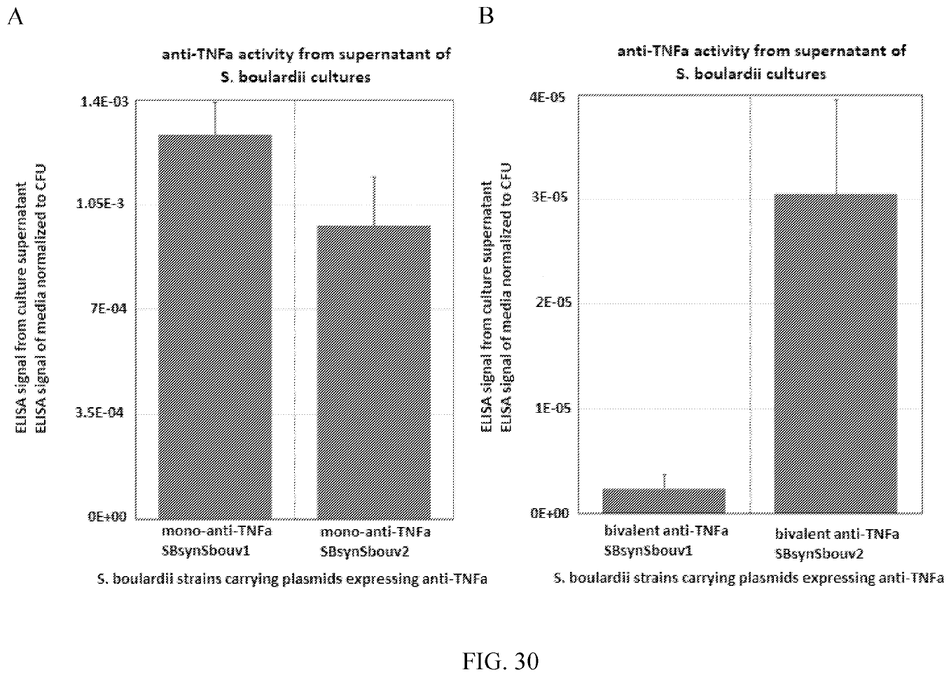

As seen in FIG. 30, S. boulardii cells have been successfully engineered to secrete anti-TNFα antibody fragments. FIG. 30 shows the amount of monovalent anti-TNFα antibody fragments secreted per CFU in yeast strains carrying plasmids with nucleic acids encoding either 6×HIS (SEQ ID NO: 105) tagged monovalent or bivalent anti-TNFα with the synthetic signal peptide comprising an amino acid represented by SEQ ID NO. 14 alone (Sbou-variant1) or fused to SEQ ID NO. 22 (Sbou-variant2). The secreted anti-TNFα antibody fragment is excreted into the growth medium. S. boulardii cultures containing plasmids comprising nucleic acids encoding each recombinant fusion protein variant were grown for 24 hours in standard YPD medium with G418 antibiotic for selection of plasmids and anti-TNFα activity was assessed in culture supernatants using the Perkin-Elmer AlphaLISA kit (Anti-6×His AlphaLISA Acceptor Beads Catalog #AL178C, Anti-FLAG Alpha Donor Beads Catalog #AS103D), using the manufacturers instructions to detect binding between anti-TNFα-6×His from supernatants and TNF-alpha-FLAG multimeric protein (Catalog #50-114-6050, Fisher Scientific). The ELISA activity from supernatants was background subtracted using growth medium and then normalized by dividing the activity by the number of CFUs, which was estimated based upon the corresponding culture's optical density at 600 nm. Based on the results shown in fugue 30 it is clear that the monovalent anti-TNFα antibody fragment shows optimal secretion with Sbou-variant 1 (FIG. 30a) whereas the bivalent anti-TNFa antibody fragment shows optimal secretion with Sbou-variant 2 (FIG. 30b). Thus S. boulardii cells were successfully engineered to secrete multiple versions of the anti-inflammatory anti-TNFa protein. Significantly, the differences observed for the two different version of the same payload may be due to the utilization of different secretion pathways within the cells, preferentially engaged based on the presence and/or absence of our synthetic pro-peptide sequences (i.e., SEQ ID 22), thus highlighting the importance of the testing multiple versions of the synthetic signal sequences as done here.

### Example 15: Enhanced Effect of Synthetic Signal Peptide on Secretion of Invertase Via Chromosomally Integrated Expression Cassettes

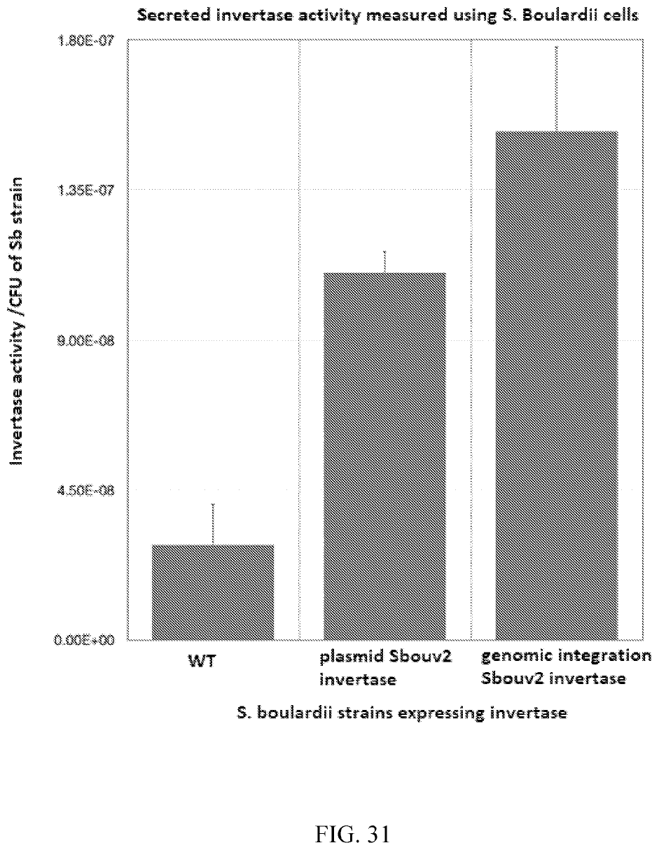

S. boulardii cells were engineered for stable and reliable expression of invertase by integrating copies of constructs containing the Sbouv2 synthetic signal peptide fused to the invertase into the S. boulardii genome. Multiple loci in the S boulardii genome were used as targets for genomic integration of the invertase expression construct and were engineered using CRISPR-Cas9 mediated gene targeting approach. The target loci used may be genes such as leu2, his3 and ura3 which exist at one location or two copies in diploid S. boulardii cells or a multi-copy locus such as the long terminal repeat (LTR) of the Ty elements in yeast genome which is present at multiple sites within the genome and hence allows for integration of multiple copies. As seen in FIG. 31 a S. boulardii strain with stably integrated invertase construct exhibits a 135% more secretion of invertase compared to a strain carrying the invertase construct on a plasmid. Thus, these manipulations allowed generation of stable S. boulardii strains, featuring no antibiotic selection markers, which exhibit higher secretion of invertase than the strains carrying the invertase expression system on a plasmid. Plasmid copy numbers change with every cell division and can lead to wide variation in expression of genes and hence could lead to unreliable expression of payloads. Plasmids also require the use of genetic selection, typically through the use of antibiotics, that is unviable and potentially unsafe for use in humans. This genomic integration approach used for expression of invertase removes these limitations, and can also easily be extended to create stable S. boulardii cells in order to achieve optimized and reliable secretion of any protein or peptide, such as all the other therapeutics described herein.

### Example 16: Effect of S. boulardii Synthetic Signal Peptide on Secretion of Luminal CCK-Releasing Factor (LCRF)

The S. boulardii optimized signal peptides with synthetic pro-protein signal peptides were tested for secretion of LCRF in S. boulardii. The LCRF peptide induces release of the peptide hormone cholecystokinin (CCK) or pancreozymin which has important roles in digestion and satiety. Nucleic acids encoding synthetic signal peptide variants (Sbou-variant 1-Sbou-variant 12) were cloned into a plasmid designed for expression of proteins in S. boulardii and the secretion of LCRF was measured. Wild type S. boulardii cells were used as control.

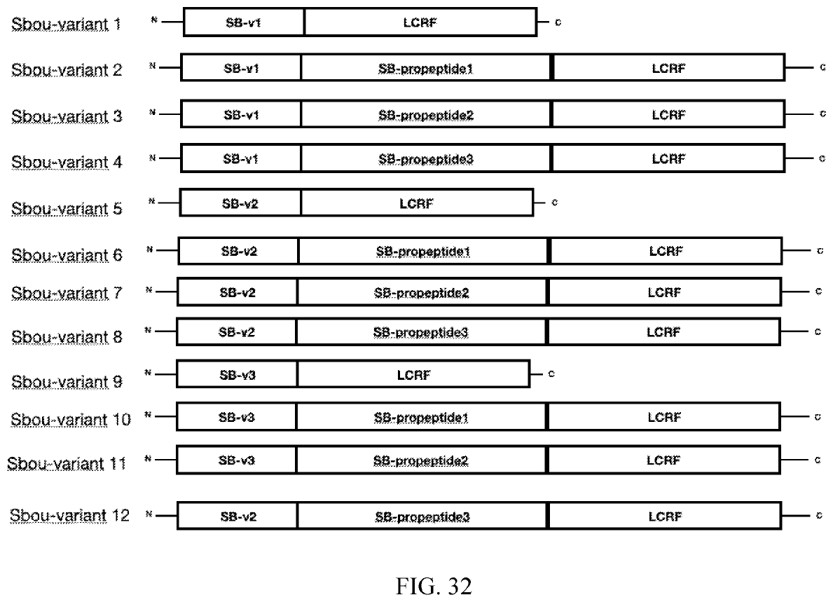

FIG. 32 illustrates the signal peptide variants tested. The pre-protein synthetic peptide utilized by Sbou-variants 1-4 comprise an amino acid sequence represented by SEQ ID NO. 14. The pre-protein synthetic peptide utilized by Sbou-variants 5-8 comprise an amino acid sequence represented by SEQ ID NO. 15. The pre-protein synthetic peptide utilized by Sbou-variants 9-12 comprise an amino acid sequence represented by SEQ ID NO. 16. Sbou-variants 1, 5, and 9 comprise no synthetic pro-protein signal peptide. Sbou-variants 2, 6, and 10 further comprise a pro-protein synthetic signal peptide comprising an amino acid sequence represented by SEQ ID NO. 22. Sbou-variants 3, 7, and 11 further comprise a pro-protein synthetic signal peptide comprising an amino acid sequence represented by SEQ ID NO. 23. Sbou-variants 5, 8, and 12 further comprise a pro-protein synthetic signal peptide comprising an amino acid sequence represented by SEQ ID NO. 24.

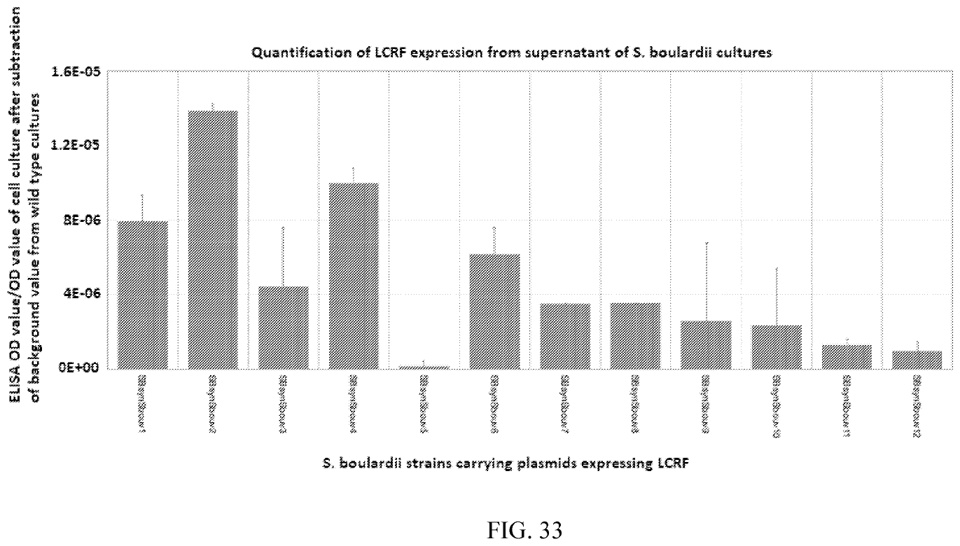

FIG. 33 shows the amount LCRF secreted per CFU in yeast strains carrying plasmids with nucleic acids encoding synthetic signal peptide variants fused to LCRF which is C-terminally tagged with the 6×HIS-3×FLAG peptide. The secreted LCRF peptide is excreted into the growth medium. S. boulardii cultures containing plasmids comprising nucleic acids encoding each recombinant fusion protein variant were grown for 24 hours in standard YPD medium with G418 antibiotic for selection of plasmids and the presence of the peptide was assayed in culture supernatants using the Perkin-Elmer AlphaLISA kit (Anti-6×His AlphaLISA Acceptor Beads Catalog #AL178C, Anti-FLAG Alpha Donor Beads Catalog #AS103D), using the manufacturers instructions to detect binding to the His & FLAG tags present on the peptides. The ELISA activity from supernatants was background subtracted using growth medium and then normalized by dividing the activity by the number of CFUs, which was estimated based upon the corresponding culture's optical density at 600 nm. The results shown in FIG. 33 indicate that optimization of the pre and pro signal peptides and their combination is important in achieving the maximal levels of secretion. This approach may provide a robust secretion from a S. boulardii cells which could be used to produce clinically relevant amounts of peptides important in digestion and satiety.

### Example 17: Exemplary Pre Peptide, Pro Peptide, and Payload Protein Combinations

As detailed herein, any pre-protein signal peptide provided can be paired with any pro-protein signal peptide provided. Additionally, any pre or pro-protein signal peptide can be used in the absence of a corresponding pro or pre-protein signal peptide, respectively. Tables 18 and 19 below recite exemplary embodiments of pre-protein signal peptide, pro-protein signal peptide, and payload protein combinations for the various embodiments described herein.

TABLE 18
Exemplary pre-peptide, pro-peptide, and payload combinations

| Combination | Pre-peptide | Pro-peptide | Payload |
|---|---|---|---|
| Number | SEQ ID | SEQ ID | SEQ ID |
| A | SEQ ID NO. 1 | — | SEQ ID NO. 64 |
| B | SEQ ID NO. 28 | SEQ ID NO. 56 | SEQ ID NO. 64 |
| C | SEQ ID NO. 28 | — | SEQ ID NO. 64 |
| D | SEQ ID NO. 1 | — | SEQ ID NO. 63 |
| E | SEQ ID NO. 28 | SEQ ID NO. 56 | SEQ ID NO. 63 |
| F | SEQ ID NO. 2 | SEQ ID NO. 17 | SEQ ID NO. 65 |
| G | SEQ ID NO. 3 | — | SEQ ID NO. 65 |
| H | SEQ ID NO. 4 | — | SEQ ID NO. 65 |
| I | SEQ ID NO. 5 | — | SEQ ID NO. 65 |
| J | SEQ ID NO. 6 | — | SEQ ID NO. 65 |
| K | SEQ ID NO. 7 | — | SEQ ID NO. 65 |
| L | SEQ ID NO. 8 | SEQ ID NO. 18 | SEQ ID NO. 66 |
| M | SEQ ID NO. 9 | SEQ ID NO. 19 | SEQ ID NO. 66 |
| N | SEQ ID NO. 10 | — | SEQ ID NO. 66 |
| O | SEQ ID NO. 9 | SEQ ID NO. 57 | SEQ ID NO. 66 |
| P | SEQ ID NO. 9 | SEQ ID NO. 58 | SEQ ID NO. 66 |
| Q | SEQ ID NO. 2 | SEQ ID NO. 25 | SEQ ID NO. 66 |
| R | SEQ ID NO. 11 | SEQ ID NO. 19 | SEQ ID NO. 66 |
| S | SEQ ID NO. 9 | SEQ ID NO. 25 | SEQ ID NO. 59 |
| T | SEQ ID NO. 13 | — | SEQ ID NO. 59 |
| U | SEQ ID NO. 9 | SEQ ID NO. 25 | SEQ ID NO. 67 |
| V | SEQ ID NO. 9 or | — | SEQ ID NO. 67 |
| | SEQ ID NO. 10 | | |
| W | SEQ ID NO. 9 | SEQ ID NO. 58 | SEQ ID NO. 67 |
| X | SEQ ID NO. 9 | SEQ ID NO. 57 | SEQ ID NO. 61 |
| Y | SEQ ID NO. 9 | SEQ ID NO. 58 | SEQ ID NO. 61 |
| Z | SEQ ID NO. 2 | SEQ ID NO. 25 | SEQ ID NO. 61 |
| AA | SEQ ID NO. 14 | — | SEQ ID NO. 59 |
| BB | SEQ ID NO. 14 | SEQ ID NO. 22 | SEQ ID NO. 59 |
| CC | SEQ ID NO. 14 | SEQ ID NO. 23 | SEQ ID NO. 59 |
| DD | SEQ ID NO. 14 | SEQ ID NO. 24 | SEQ ID NO. 59 |
| EE | SEQ ID NO. 15 | — | SEQ ID NO. 59 |
| FF | SEQ ID NO. 15 | SEQ ID NO. 22 | SEQ ID NO. 59 |
| GG | SEQ ID NO. 15 | SEQ ID NO. 23 | SEQ ID NO. 59 |
| HH | SEQ ID NO. 15 | SEQ ID NO. 24 | SEQ ID NO. 59 |
| II | SEQ ID NO. 16 | — | SEQ ID NO. 59 |
| JJ | SEQ ID NO. 16 | SEQ ID NO. 22 | SEQ ID NO. 59 |
| KK | SEQ ID NO. 16 | SEQ ID NO. 23 | SEQ ID NO. 59 |
| LL | SEQ ID NO. 16 | SEQ ID NO. 24 | SEQ ID NO. 59 |
| MM | SEQ ID NO. 14 | — | SEQ ID NO. 61 |
| NN | SEQ ID NO. 14 | SEQ ID NO. 22 | SEQ ID NO. 61 |
| OO | SEQ ID NO. 14 | SEQ ID NO. 23 | SEQ ID NO. 61 |
| PP | SEQ ID NO. 14 | SEQ ID NO. 24 | SEQ ID NO. 61 |
| QQ | SEQ ID NO. 15 | — | SEQ ID NO. 61 |
| RR | SEQ ID NO. 15 | SEQ ID NO. 22 | SEQ ID NO. 61 |
| SS | SEQ ID NO. 15 | SEQ ID NO. 23 | SEQ ID NO. 61 |
| TT | SEQ ID NO. 15 | SEQ ID NO. 24 | SEQ ID NO. 61 |
| UU | SEQ ID NO. 16 | — | SEQ ID NO. 61 |
| VV | SEQ ID NO. 16 | SEQ ID NO. 22 | SEQ ID NO. 61 |
| XX | SEQ ID NO. 16 | SEQ ID NO. 23 | SEQ ID NO. 61 |
| YY | SEQ ID NO. 16 | SEQ ID NO. 24 | SEQ ID NO. 61 |
| ZZ | SEQ ID NO. 55 | — | SEQ ID NO. 61 |
| AAA | SEQ ID NO. 14 | SEQ ID NO. 22 | SEQ ID NO. 62 |
| BBB | SEQ ID NO. 14 | — | SEQ ID NO. 63 |
| CCC | SEQ ID NO. 14 | SEQ ID NO. 22 | SEQ ID NO. 63 |

TABLE 19
Further exemplary pre-peptide, pro-
peptide, and payload combinations

| | | | |
|---|---|---|---|
| Combination | Pre-peptide | Pro-peptide | Payload |
| Number | SEQ ID | SEQ ID | SEQ ID |

SEQ ID NO. 59 Exemplary Combinations

| | | | |
|---|---|---|---|
| DDD | SEQ ID 14 | SEQ ID NO. 22 | SEQ ID NO. 59 |
| EEE | SEQ ID 14 | SEQ ID NO. 24 | SEQ ID NO. 59 |
| FFF | SEQ ID 14 | SEQ ID NO. 23 | SEQ ID NO. 59 |

SEQ ID NO. 61 Exemplary Combinations

| | | | |
|---|---|---|---|
| GGG | SEQ ID NO. 14 | — | SEQ ID NO. 61 |
| HHH | SEQ ID NO. 15 | — | SEQ ID NO. 61 |
| III | SEQ ID NO. 14 | SEQ ID NO. 23 | SEQ ID NO. 61 |
| JJJ | SEQ ID NO. 14 | SEQ ID NO. 22 | SEQ ID NO. 61 |
| KKK | SEQ ID NO. 15 | SEQ ID NO. 22 | SEQ ID NO. 61 |
| LLL | SEQ ID NO. 14 | SEQ ID NO. 24 | SEQ ID NO. 61 |
| MMM | SEQ ID NO. 16 | SEQ ID NO. 23 | SEQ ID NO. 61 |
| NNN | SEQ ID NO. 15 | SEQ ID NO. 24 | SEQ ID NO. 61 |
| OOO | SEQ ID NO. 16 | SEQ ID NO. 22 | SEQ ID NO. 61 |
| PPP | SEQ ID NO. 16 | SEQ ID NO. 24 | SEQ ID NO. 61 |
| QQQ | SEQ ID NO. 14 | — | SEQ ID NO. 61 |

SEQ ID NO. 62 Exemplary Combinations

| | | | |
|---|---|---|---|
| RRR | SEQ ID NO. 14 | SEQ ID NO. 22 | SEQ ID NO. 62 |

Monovalent SEQ ID NO. 63 Exemplary Combinations

| | | | |
|---|---|---|---|
| SSS | SEQ ID NO. 14 | — | SEQ ID NO. 63 |
| TTT | SEQ ID NO. 14 | SEQ ID NO. 22 | SEQ ID NO. 63 |

Bivalent SEQ ID NO. 63 Exemplary Combinations

| | | | |
|---|---|---|---|
| UUU | SEQ ID NO. 14 | SEQ ID NO. 22 | SEQ ID NO. 63 |
| VVV | SEQ ID NO. 14 | — | SEQ ID NO. 63 |

SEQ ID NO. 85 Exemplary Combinations

| | | | |
|---|---|---|---|
| WWW | SEQ ID NO. 14 | SEQ ID NO. 22 | SEQ ID NO. 85 |
| XXX | SEQ ID NO. 14 | SEQ ID NO. 24 | SEQ ID NO. 85 |
| | SEQ ID NO. 14 | — | SEQ ID NO. 85 |
| ZZZ | SEQ ID NO. 15 | SEQ ID NO. 22 | SEQ ID NO. 85 |
| AAAA | SEQ ID NO. 14 | SEQ ID NO. 23 | SEQ ID NO. 85 |
| BBBB | SEQ ID NO. 15 | SEQ ID NO. 24 | SEQ ID NO. 85 |
| CCCC | SEQ ID NO. 15 | SEQ ID NO. 23 | SEQ ID NO. 85 |
| DDDD | SEQ ID NO. 16 | — | SEQ ID NO. 85 |
| EEEE | SEQ ID NO. 16 | SEQ ID NO. 22 | SEQ ID NO. 85 |
| FFFF | SEQ ID NO. 16 | SEQ ID NO. 23 | SEQ ID NO. 85 |
| GGGG | SEQ ID NO. 16 | SEQ ID NO. 24 | SEQ ID NO. 85 |
| HHHH | SEQ ID NO. 15 | — | SEQ ID NO. 85 |

### Example 18: Use of Engineered Yeast for Prevention and Treatment of Insect Infested Plants

To test the compatibility of engineered yeast as vectors for the prevention of insect infestation in plants, engineered yeast will be generated expressing a recombinant polypeptide comprising insecticides (e.g. Vip1, Vip2, Vip3, Cry proteins, and the like) and one or both of a pre-protein signal peptide as provided for herein and a pro-protein signal peptide as provided for herein. Different combinations will be constructed to provide for the optimal pre and pro protein peptide combination. Plants will be sprayed with yeast expressing the recombinant polypeptide or a control composition and will be allowed to settle. After a pre-determined amount of time, plants from each group will be subject to insect exposure and the ability of the yeast expressing insecticides to prevent insect related damage and infestation will be assessed.

Similarly, the engineered yeast will be assessed for their ability to treat an existing insect infestation. Engineered yeast will be generated expressing a recombinant polypeptide comprising insecticides (e.g. Vip1, Vip2, Vip3, Cry proteins, and the like) and one or both of a pre-protein signal peptide as provided for herein and a pro-protein signal peptide as provided for herein. Different combinations will be constructed to provide for the optimal pre and pro protein peptide combination. Plants will be subject to insect exposure for a pre-determined period of time. Once an infestation is established, plants will be sprayed with either a control composition or a composition comprising the engineered yeast described in this example. The ability of the yeast to clear the existing insect infestation will be assessed.

It should be recognized that illustrated embodiments are only examples of the disclosed product and methods and should not be considered a limitation on the scope of the invention. Rather, the scope of the invention is defined by the following claims. We therefore claim as our invention all that comes within the scope and spirit of these claims.

1. A pre-protein signal peptide comprising an amino acid sequence selected from the group consisting of Formula I, II, III, IV, V, IX, and XIII;
wherein Formula I is represented as:
A₁-(A₂)_{w}-A₃-(A₄)ₓ-(A₅)_{y}-A₆-A₇-A₈-A₉-A₁₀-(A₁₁)_{z} (Formula I)
wherein:
w and x are each, independently, 1, 2, 3, 4, or 5;
y is 2, 3, 4, 5, 6, 7, 8, 9, 10, 11, 12, 13, 14, 15, 16, 17, 18, 19, or 20; and
z is 1, 2, or 3;
wherein:
A₁ is methionine;
each A₂ is, independently, a neutral or positively-charged amino acid with a hydropathy index of less than about 1;
each A₃, A₅, A₈, and A₁₀ is each, independently, an amino acid with a hydropathy index greater than −1, excluding W and C;
each A₄ is, independently, a basic or neutral amino acid, excluding P, W, M, and C;
A₆ is an amino acid with a hydropathy index greater than −1, excluding W, M, and C;
A₇ is a non-aromatic amino acid with a hydropathy index of less than about 1.9 and an isoelectric point of about 5.4 to about 7.5, excluding P;
A₉ is an amino acid with a hydropathy index of greater than about −1.3; and
each A₁₁ is, independently, a neutral amino acid with a molecular weight of less than about 133 g/mol;
wherein Formula II is represented as:
B₁-(B₂)ᵤ-(B₃)ᵥ-(B₄)_{w}-(B₅)ₓ-(B₃)_{y}-B₇-B₈-B₉-B₁₀-(B₁₁)_{z} (Formula II)
wherein:
u and w are each, independently, 0, 1, 2, or 3;
v and z are each, independently, 1, 2, or 3;
x is 0, 1, or 2; and
y is 2, 3, 4, 5, 6, 7, 8, 9, 10, 11, 12, 13, 14, 15, 16, 17, 18, 19, or 20;
wherein:
B₁ is methionine;
each B₂, B₄, B₆, B₈ and B₁₀ is each, independently, an amino acid with a hydropathy index of greater than about −1, excluding W and C;
each B₃ is, independently, a positively-charged or polar amino acid with a hydropathy index of less than about 1;
each B₅ is, independently, a polar amino acid with a hydropathy index of greater than about −5 and less than about −0.5, or an amino acid with an isoelectric point of about 5 to about 11, excluding P, W, M, and C;
each B₇ and B₁₁ is each, independently, a neutral amino acid with a molecular weight of less than about 133 g/mol; and
B₉ is an amino acid with a hydropathy index of greater than about −1.3;
wherein Formula III is represented as:
C₁-(C₂)ᵣ-(C₃)ₜ-(C₄)ᵤ-[(C₅)ᵥ-(C₆)_{w}]ₓ-(C₇)_{y}-(C₈)_{z}-C₉-C₁₀-C₁₁-[C₁₂-C₁₃]ₐ (Formula III)
wherein:
r is 1, 2, or 3;
t, u, y, and z are each, independently, 0, 1, 2, or 3;
v and w are each, independently, 0, 1, or 2;
a is 0 or 1; and
x is 2, 3, 4, 5, 6, 7, 8, 9, or 10;
wherein:
C₁ is methionine;
each C₂ is, independently, an amino acid having an isoelectric point of about 5.6 to about 10.8, a molecular weight of about 105 g/mol to about 175 g/mol, a hydropathy index of about −5.1 to about 0.6, and a helicity of about 0.8 to about 1;
each C₃, C₅, C₈, and C₁₀ is each, independently, an amino acid having an isoelectric point of about 2.75 to about 10.8, a molecular weight of about 75 g/mol to about 205 g/mol, a hydropathy index of about −5.1 to about 34, and a helicity of about 0.5 to about 1.3;
each C₄ and C₇ is each, independently, an amino acid having an isoelectric point of about 5 to about 10.8, a molecular weight of about 75 g/mol to about 205 g/mol, a hydropathy index of about −5.1 to about 34, and a helicity of about 0.5 to about 1.3;
each C₆, C₉, C₁₁, and C₁₂ is each, independently, an amino acid having an isoelectric point of about 2.75 to about 9.8, a molecular weight of about 75 g/mol to about 205 g/mol; a hydropathy index of about −4 to about 34, and a helicity of about 0.5 to about 1.3; and
C₁₃ is an amino acid having an isoelectric point of about 5.6 to about 6.3, a molecular weight of about 105 g/mol to about 120 g/mol, a hydropathy index of about 0 to about 9.4, and a helicity of about 0.5 to about 1.1;
wherein Formula IV is represented as:
D₁-(D₂)_{q}-(D₃)ᵣ-(D₄)ₜ-(D₅)ᵤ-[(D₆)ᵥ-(D₇)ₓ-(D₈)_{w}-(D₉)_{y}]_{z}-D₁₀-Dᵤ-D₁₂-[D₁₃-D₁₄]ₐ (Formula IV)
wherein:
q is 1, 2, or 3;
r, t, and u are each, independently, 0, 1, 2, or 3;
v, w, x, and y are each, independently, 0, 1, or 2;
a is 0 or 1; and
z is 2, 3, 4, 5, 6, 7, 8, 9, or 10;
wherein:
D₁ is methionine;
each D₂ is, independently, an amino acid having an isoelectric point of about 2.7 to about 10.8, a molecular weight of about 75 g/mol to about 205 g/mol, a hydropathy index of about −5.1 to about 34, and a helicity of about 0.5 to about 1.3;
each D₃ is, independently, an amino acid having an isoelectric point of about 5 to about 10.8, a molecular weight of about 89 g/mol to about 205 g/mol, a hydropathy index of about −4 to about 34, and a helicity of about 0.5 to about 1.3;
each D₄, D₉ and D₁₁ is each, independently an amino acid having an isoelectric point of about 2.7 to about 10.8, a molecular weight of about 75 g/mol to about 205 g/mol, a hydropathy index of about −5.1 to about 34, and a helicity of about 0.5 to about 1.3;
each D₅ is, independently, an amino acid having an isoelectric point of about 3.2 to about 10.8, a molecular weight of about 75 g/mol to about 205 g/mol, a hydropathy index of about −5.1 to about 34, and a helicity of about 0.75 to about 1.3;
each D₆ is, independently, an amino acid having an isoelectric point from about 5 to about 10.8, a molecular weight of about 75 g/mol to about 205 g/mol, a hydropathy index of about −5.1 to about 34, and a helicity of about 0.5 to about 1.3;
each D₇ is, independently, an amino acid having an isoelectric point of about 5.4 to about 6.1, a molecular weight of about 117 g/mol to about 205 g/mol, a hydropathy index of about 2.5 to about 34, and a helicity of about 1 to about 1.3;
each D₈, D₁₀, D₁₂, and D₁₃ is each, independently, an amino acid having an isoelectric point of about 2.7 to about 10.8, a molecular weight of about 75 g/mol to about 182 g/mol, a hydropathy index of about −5.1 to about 32, and a helicity of about 0.75 to about 1.3; and
D₁₄ is an amino acid with an isoelectric point of about 2.7 to about 10.8, a molecular weight of about 75 g/mol to about 182 g/mol, a hydropathy index of about −5.1 to about 32, and a helicity of about 0.5 to about 1.3;
wherein Formula V is represented as:
E₁-[(E₂)ᵢ-(E₃)ⱼ-(E₄)_{q}]ᵣ-(E₅)ₜ-(E₆)ᵤ-(E₇)ᵥ-[(E₈)_{w}-(E₉)ₓ]_{y}-(E₁₀)_{z}-E₁₁-E₁₂-E₁₃-[E₁₄-E₁₅]ₐ (Formula V)
wherein:
i, j, q, w, x and a are each, independently, 0 or 1;
r is 1, 2, or 3;
t, u, v, and z are each, independently, 0, 1, 2, or 3; and
y is 2, 3, 4, 5, 6, 7, 8, 9, or 10;
wherein:
E₁ is methionine;
each E₂ is, independently, an amino acid having an isoelectric point of about 3.2 to about 10.8, a molecular weight of about 105 g/mol to about 175 g/mol, a hydropathy index of about −4 to about 1, and a helicity of about 0.85 to about 1;
each E₃ is, independently, an amino acid having an isoelectric point of about 2.7 to about 10.8, a molecular weight of about 75.1 g/mol to about 205 g/mol, a hydropathy index of about −5.1 to about 33.5, and a helicity of about 0.57 to about 1.3;
each E₄ is, independently, an amino acid having an isoelectric point of about 5 to about 10.8, a molecular weight of about 105 g/mol to about 205 g/mol, a hydropathy index of about −5.1 to about 33.5, and a helicity of about 0.57 to about 1.3;
each E₅ and E₈ is each, independently, an amino acid having an isoelectric point of about 2.7 to about 10.8, a molecular weight of about 75 g/mol to about 205 g/mol, a hydropathy index of about −5.1 to about 33.5, and a helicity of about 0.57 to about 1.3;
each E₆ is, independently, an amino acid having an isoelectric point of about 5 to about 10.8, a molecular weight of about 89 g/mol to about 205 g/mol, a hydropathy index of about −5.1 to about 33.5, and a helicity of about 0.57 to about 1.3;
each E₇ is, independently, an amino acid having an isoelectric point of about 5 to about 9.75, a molecular weight of about 75 g/mol to about 205 g/mol, a hydropathy index of about −4 to about 33.5, and a helicity of about 0.79 to about 1.3;
each E₉, E₁₃, and E₁₄ is each, independently, an amino acid having an isoelectric point of about 2.7 to about 10.8, a molecular weight of about 75 g/mol to about 205 g/mol, a hydropathy index of about −5.1 to about 33.5, and a helicity of about 0.57 to about 1.3;
each E₁₀ and E₁₂ is, independently, an amino acid having an isoelectric point of about 5 to about 10.8, a molecular weight of about 75 g/mol to about 205 g/mol, a hydropathy index of about −5.1 to about 34, and a helicity of about 0.5 to about 1.3;
E₁₁ is an amino acid having an isoelectric point of about 5 to about 9.75, a molecular weight of about 89 g/mol to about 205 g/mol, a hydropathy index of about −4 to about 33.5, and a helicity of about 0.79 to about 1.3; and
E₁₅ is an amino acid having an isoelectric point of about 2.7 to about 10.8, a molecular weight of about 75 g/mol to about 205 g/mol, a hydropathy index of about −4 to about 15.5, and a helicity of about 0.57 to about 1.2;
wherein Formula IX is represented as:
F₁-(F₂)ᵥ-(F₃)_{w}-[(F₄)ₓ-(F₅)_{y}]_{z}-F₆-F₇-F₈-[F₉-F₁₀]ₐ (Formula IX)
wherein:
v and w are each, independently, 0, 1, 2, or 3;
x and y are each, independently, 0, 1, 2, 3, or 4;
a is 0 or 1; and
z is 1, 2, 3, 4, 5, 6, 7, or 8;
wherein:
F₁ is an amino acid having an isoelectric point of about 5.4 to about 11, a molecular weight of about 89 g/mol to about 175 g/mol; a hydropathy index of about −4 to about 31, and a helicity or about 0.9 to about 1.3;
each F₂ is, independently, an amino acid having an isoelectric point of about 3 to about 11, a molecular weight of about 89 g/mol to about 205 g/mol; a hydropathy index of about −5.1 to about 34, and a helicity or about 0.5 to about 1.3;
each F₃ and F₇ is each, independently, an amino acid having an isoelectric point of about 3 to about 11, a molecular weight of about 89 g/mol to about 205 g/mol; a hydropathy index of about −5.1 to about 34, and a helicity or about 0.5 to about 1.3;
each F₄ is, independently, an amino acid having an isoelectric point of about 3 to about 11, a molecular weight of about 89 g/mol to about 205 g/mol; a hydropathy index of about −5.1 to about 34, and a helicity or about 0.5 to about 1.3;
each F₅, F₆, F₈, and F₉ is each, independently, an amino acid having an isoelectric point of about 3 to about 11, a molecular weight of about 89 g/mol to about 205 g/mol; a hydropathy index of about −5.1 to about 34, and a helicity or about 0.5 to about 1.3; and
F₁₀ is an amino acid having an isoelectric point of about 3 to about 11, a molecular weight of about 89 g/mol to about 205 g/mol; a hydropathy index of about −5.1 to about 34, and a helicity or about 0.5 to about 1.3; and
wherein Formula XIII is represented as:
L₁-(L₂)ₓ[(L₃)ₐ-(L₄)ₐ]_{y}-[(L₅)ₐ-(L₆)ₐ-(L₇)ₐ]_{z}-(L₈)ₐ-(L₉)ₐ-(L₁₀)ₐ-(L₁₁)ₐ-(L₁₂)ₐ (Formula XIII)
wherein:
x is 1, 2, or 3;
y is 1, 2, 3, or 4;
z is 5, 6, 7, 8, 9, or 10; and
each a is, independently, 0 or 1;
wherein:
each L₂ is, independently, an amino acid having an isoelectric point of about 2.7 to about 10.8, a molecular weight of about 75 g/mol to about 205 g/mol; a hydropathy index of about −5.1 to about 34, and a helicity or about 0.5 to about 1.3;
each L₃ and L₆ is each, independently, an amino acid having an isoelectric point of about 2.7 to about 10.8, a molecular weight of about 75 g/mol to about 205 g/mol; a hydropathy index of about −5.1 to about 34, and a helicity or about 0.5 to about 1.3;
each L₄, L₇ and L₉ is each, independently, an amino acid having an isoelectric point of about 2.7 to about 10.8, a molecular weight of about 75 g/mol to about 205 g/mol; a hydropathy index of about −5.1 to about 34, and a helicity or about 0.5 to about 1.3;
each L₅, L₈, L₁₀ and Ln is each, independently, an amino acid having an isoelectric point of about 2.7 to about 10.8, a molecular weight of about 75 g/mol to about 205 g/mol; a hydropathy index of about −5.1 to about 34, and a helicity or about 0.5 to about 1.3; and
L₁₂ is an amino acid having an isoelectric point of about 2.7 to about 10.8, a molecular weight of about 75 g/mol to about 205 g/mol; a hydropathy index of about −5.1 to about 34, and a helicity or about 0.5 to about 1.3.

2. The pre protein signal peptide of claim 1, wherein for Formula I:
each A₂ is, independently, an amino acid selected from the group consisting of K, R, and Q;
each A₃, A₅, A₈, and A₁₀ is each, independently, an amino acid selected from the group consisting of L, V, A, and I; and
each A₁₁ is, independently, an amino acid selected from the group consisting of A, L, and G.

3. The pre protein signal peptide of claim 1, wherein for Formula II:
each B₂, B₄, B₆, B₈ and B₁₀ is each, independently, an amino acid selected from the group consisting of L, V, A, F, and I;
each B₃ is, independently, an amino acid selected from the group consisting of K, R, and Q; and
each B₇ and B₁₁ is, independently, an amino acid selected from the group consisting of A, S, G, and P.

4. The pre protein signal peptide of claim 1, wherein for Formula III:
each C₂ is, independently, an amino acid selected from the group consisting of K, R, H, S, and Q;
each C₃, C₅, C₈, and C₁₀ is each, independently, an amino acid selected from the group consisting of L, V, I, A, W, Y, T, Q, S, H, C, N, D, R, P, K, G, E, and M;
each C₄ and C₇ is each, independently, an amino acid selected from the group consisting of S, N, Q, R, T, K, A, Y, H, V, I, F, G, W, C, P, and L;
each C₆, C₉, C₁₁, and C₁₂ is each, independently, an amino acid selected from the group consisting of A, S, V, G, I, L, F, C, T, K, P, Q, N, Y, E, D, M, and W; and
C₁₃ is an amino acid selected from the group consisting of P, T, and S.

5. The pre protein signal peptide of claim 1, wherein for Formula IV:
each D₂ is, independently, an amino acid selected from the group consisting of K and R;
each D₃ is, independently, an amino acid selected from the group consisting of F, L, I, W, V, M, Y, P, C, A, Q, and S;
each D₄, D₉ and D₁₁ is each, independently an amino acid selected from the group consisting of L, I, F, W, V, M, Y, A, T, N, S, G, E, D, C, Q, R, H, P, and K;
each D₅ is, independently, an amino acid selected from the group consisting of S, N, Q, R, T, G, K, E, H, A, C, Y, V, W, I, F, and L;
each D₆ is, independently, an amino acid selected from the group consisting of L, I, A, T, S, G, N, R K, Y Q, C, H, W, and M;
each D₇ is, independently, an amino acid selected from the group consisting of V, W, I, L, F, and T;
each D₈, D₁₀, D₁₂, and D₁₃ is each, independently, an amino acid selected from the group consisting of A, S, T, G, V, L, C, Y, K, I, F, Q, N, H, R, E, D, and M; and
D₁₄ is an amino acid selected from the group consisting of P, Y, M, V, A, T, Q, S, N, G, I, E, D, L, F, R, K, and H.

6. The pre protein signal peptide of claim 1, wherein for Formula V:
each E₂ is, independently, an amino acid selected from the group consisting of K, R, S, Q, and E;
each E₃ is, independently, an amino acid selected from the group consisting of F, L, I, W, V, Y, P, A, T, Q, N, S, G, D, R, K, and H;
each E₄ is, independently, an amino acid selected from the group consisting of K, R, H, S, C, P, Y, M, V, W, I, L, and F;
each E₅ and E₈ is each, independently, an amino acid selected from the group consisting of L, I, F, V, C, A, Y, T, Q, N, S, K, H, W, G, D, M, P, E, and R;
each E₆ is, independently, an amino acid selected from the group consisting of T, Q, S, A, C, R, K, H, P, V, W, I, F, and L;
each E₇ is, independently, an amino acid selected from the group consisting of S, G, K, A, C, Y, V, and W;
each E₉, E₁₃, and E₁₄ is each, independently, an amino acid selected from the group consisting of A, T, G, S, V, I, L, Y, W, F, C, Q, N, P, E, M, R, K, D, and H;
each E₁₀ and E₁₂ is each, independently, an amino acid selected from the group consisting of L, F, I, V, C, Y, T, Q, N, S, K, H, M, G, A, W, D, P, E, and R.
E₁₁ is an amino acid selected from the group consisting of V, W, I, C, L, A, T, S, and K; and
E₁₅ is an amino acid selected from the group consisting of S, N, R, T, G, K, E, D, P, and Y.

7. The pre-protein signal peptide of claim 1 wherein for Formula IX:
F₁ is an amino acid selected from the group consisting of M, F, L, A, S, or R;
each F₂ is, independently, an amino acid selected from the group consisting of K, R, H, S, G, N, Q, E, T, A, C, P, Y, V, W, I, L, or F;
each F₃ and F₇ is each, independently, an amino acid selected from the group consisting of S, Q, R, T, K, H, I, F, L, P, N, G, E, D, A, Y, M, V, W, or C;
each F₄ is, independently, an amino acid selected from the group consisting of L, I, V, M, A, F, W, Y, P, C, T, Q, N, S, G, E, R, K, or H;
each F₅, F₆, F₈, and F₉ is each, independently, an amino acid selected from the group consisting of A, C, G, S, V, L, T, F, Q, N, P, Y, E, K, H, W, I, M, R, or D; and
F₁₀ is an amino acid selected from the group consisting of P, C, Y, M, V, A, T, Q, S, N, W, G, I, E, D, L, F, R, K, or H.

8. The pre-protein signal peptide of claim 1 wherein for Formula XIII:
each L₂ is, independently, an amino acid selected from the group consisting of R, K, H, S, G, N, Q, D, T, A, C, P, Y, M, V, W, I, F, and L;
each L₃ and L₆ is each, independently, an amino acid selected from the group consisting of S, N, Q, R, T, K, P, G, E, H, D, A, C, Y, M, V, W, I, F, and L;
each L₄, L₇ and L₉ is each, independently, an amino acid selected from the group consisting of L, F, I, W, V, T, M, Y, P, C, A, Q, N, S, G, E, D, R, K, and H;
each L₅, L₈, L₁₀ and L₁₁ is each, independently, an amino acid selected from the group consisting of A, T, G, S, C, P, I, L, F, R, V, Q, Y, K, N, E, D, H, M, and W; and
L₁₂ is an amino acid selected from the group consisting of P, T, S, D, C, Y, M, V, A, Q, N, W, G, I, E, L, F, R, K, and H.

9. The pre-protein signal peptide of claim 1, wherein the signal peptide comprises an amino acid sequence having at least 90%, 91%, 92%, 93%, 94%, 95%, 96%, 97%, 98%, or 99% identity to an amino acid sequence selected from the group consisting of SEQ ID NO. 1, 2, 3, 4, 5 6, 7, 8, 9, 10, 11, 12, 13, 14, 15, 16, 31, 32, 33, 70, 71, 72, and 73.

10. The pre-protein signal peptide of claim 1, wherein the amino acid sequence is selected from the group consisting of SEQ ID NO. 1, 2, 3, 4, 5, 6, 7, 8, 9, 10, 11, 12, 13, 14, 15, 16, 28, 31, 32, 33, 55, 70, 71, 72, or 73.

11. A pre-protein signal peptide comprising an amino acid sequence having at least 90%, 910%, 92%, 93%, 94%, 95%, 96%, 97%, 98%, 99% or 100% identity to SEQ ID NO. 1, 2, 3, 4, 5, 6, 7, 8, 9, 10, 11, 12, 13, 14, 15, 16, 28, 31, 32, 33, 55, 70, 71, 72, or 73.

12. A pro-protein signal peptide comprising an amino acid sequence selected from the group consisting of Formula VI, VII, VIII, X, XI, XIV, and XV;
wherein Formula VI is represented as:
G₁-G₂-G₃-G₄-G₅-G₆-G₇-G₈-G₉-G₁₀-G₁₁-G₁₂-G₁₃-G₁₄-G₁₅-G₁₆-G₁₇-G₁₈-G₁₉-G₂₀-G₂₁-G₂₂-G₂₃-G₂₄-G₂₅ (Formula VI)
wherein:
G₁ is an amino acid selected from the group consisting of I, L, F, V, A, N, S, D, R, and K;
G₂ is an amino acid selected from the group consisting of P, S, N, G, and E;
G₃ is an amino acid selected from the group consisting of L, F, I, V, Y, A, S, R, and H;
G₄ is an amino acid selected from the group consisting of V, M, P, Y, A, T, S, N, K, and H;
G₅ is an amino acid selected from the group consisting of A, G, R, Y, K, D, M, V, W, I, and L;
G₆ is an amino acid selected from the group consisting of N, R, and K;
G₇ is an amino acid selected from the group consisting of V, P, A, T, Q, G, E, D, R, and K;
G₈ is an amino acid selected from the group consisting of P, Y, T, Q, S, N, W, F, R, K, and H;
G₉ is an amino acid selected from the group consisting of F, L, A, Q, N, S, E, G, D, and H;
G₁₀ is an amino acid selected from the group consisting of H, S, N, D, Q, E, T, Y, M, V, I, and L;
G₁₁ is an amino acid selected from the group consisting of S, R, T, G, K, E, D, and P;
G₁₂ is an amino acid selected from the group consisting of D, E, Q, N, A, and V;
G₁₃ is an amino acid selected from the group consisting of N, S, E, D, T, H, K, A, and P;
G₁₄ is an amino acid selected from the group consisting of G, S, N, H, E, C, Y, L, and F;
G₁₅ is an amino acid selected from the group consisting of S, T, and H;
G₁₆ is an amino acid selected from the group consisting of E, D, Q, N, S, T, K, and A;
G₁₇ is an amino acid selected from the group consisting of W, N, D, and R;
G₁₈ is an amino acid selected from the group consisting of L and F;
G₁₉ is an amino acid selected from the group consisting of Y, V, A, Q, N, S, E, D, L, R, K, and H;
G₂₀ is an amino acid selected from the group consisting of K, R, S, and I;
G₂₁ is R;
G₂₂ is an amino acid selected from the group consisting of D, E, N, S, T, G, A, Y, and L;
G₂₃ and G₂₄ are each, independently, an amino acid selected from the group consisting of V, P, Y, I, A, E, K, F, T, S, G, D, M, and N; and
G₂₅ is an amino acid selected from the group consisting of Y, P, A, T, Q, S, E, F, and H;
wherein Formula VII is represented as:
(H₁)ₘ-(H₂)ₘ-(H₃)ₘ-(H₄)ₘ-(H₅)ₘ-(H₆)ₘ-(H₇)ₘ-(H₈)ₘ-(H₉)ₘ-(H₁₀)ₘ-(H₁₁)ₘ- (H₁₂)ₘ-(H₁₃)ₘ-(H₁₄)ₘ-(H₁₅)ₘ-(H₁₆)ₘ-(H₁₇)ₘ-(H₁₈)ₘ-(H₁₉)ₘ-(H₂₀)ₘ-(H₂₁)ₘ-(H₂₂)ₘ-(H₂₃)ₘ-(H₂₄)ₘ-(H₂₅)ₘ-(H₂₆)ₘ-(H₂₇)ₘ-(H₂₈)ₘ-(H₂₉)ₘ-(H₃₀)ₘ-(H₃₁)ₘ-(H₃₂)ₘ-(H₃₃)ₘ-(H₃₄)ₘ-(H₃₅)ₘ-(H₃₆)ₘ-H₃₇-H₃₈-H₃₉-H₄₀ (Formula VII)
wherein:
each m is, independently, 0, 1, or 2;
wherein:
each H₁ is, independently, an amino acid selected from the group consisting of E, D, S, L, G, Q, and A;
each H₂ and H₂₈ is each, independently, an amino acid selected from the group consisting of P, S, R, T, N, G, D, K, and A;
each H₃ is, independently, an amino acid selected from the group consisting of W and Y;
each H₄ is, independently, an amino acid selected from the group consisting of S, N, A, P, and V;
each H₅ and H₃₀ is each, independently, an amino acid selected from the group consisting of T, Q, A, E, F, and S;
each H₆ is, independently, an amino acid selected from the group consisting of L, F, and I;
each H₇ is, independently, an amino acid selected from the group consisting of F, V, M, T, S, and K;
each H₈ is, independently, an amino acid selected from the group consisting of V, P, I, A, S, and K;
each H₉ and H₁₇ is each, independently, an amino acid selected from the group consisting of T, G, V, W, and A;
each H₁₀ is, independently, an amino acid selected from the group consisting of R, H, S, G, N, E, T, and V;
each H₁₁ is, independently, an amino acid selected from the group consisting of S, G, D, A, and M;
each H₁₂ is, independently, an amino acid selected from the group consisting of T, S, E, G, D, K, and H;
each H₁₃ is, independently, an amino acid selected from the group consisting of L, M, Y, N, S, D, and K;
each H₁₄ is, independently, an amino acid selected from the group consisting of D, Q, N, S, K, and C;
each H₁₅ is, independently, an amino acid selected from the group consisting of E, S, D, L, and G;
each H₁₆ is, independently, an amino acid selected from the group consisting of I, L, V, M, A, and T;
each H₁₈ is, independently, an amino acid selected from the group consisting of D, E, S, T, K, and G;
each H₁₉ is, independently, an amino acid selected from the group consisting of Y, F, and L;
each H₂₀ is, independently, an amino acid selected from the group consisting of N, Q, S, T, R, and F;
each H₂₁ and H₃₄ is each, independently, an amino acid selected from the group consisting of S, K, T, A, Y, M, and F;
each H₂₂ is, independently, an amino acid selected from the group consisting of T, Q, S, D, C, V, and L;
each H₂₃ is, independently, an amino acid selected from the group consisting of G, S, K, N, H, D, W, and L;
each H₂₄ is, independently, an amino acid selected from the group consisting of I, L, V, P, N, and E;
each H₂₅ and H₃₃ is each, independently, an amino acid selected from the group consisting of A, T, G, R, Y, L, F, and E;
each H₂₆ and H₄₀ is each, independently, an amino acid selected from the group consisting of V, I, F, M, L, A, and T;
each H₂₇ is, independently, an amino acid selected from the group consisting of D, E, Q, N, S, A, and I;
each H₂₉ is, independently, an amino acid selected from the group consisting of E, D, T, A, Y, M, V, I, F, and L;
each H₃₁ is, independently, an amino acid selected from the group consisting of F, W, V, M, S, G, and R;
each H₃₂ is, independently, an amino acid selected from the group consisting of H, S, E, G, and T;
each H₃₅ is, independently, an amino acid selected from the group consisting of R, K, S, and Q;
each H₃₆ is, independently, an amino acid selected from the group consisting of H, R, S, T, A, V, W, and L;
H₃₇ is an amino acid selected from the group consisting of K, Q, D, A, and I;
H₃₈ is an amino acid selected from the group consisting of R, K, T, and F; and
H₃₉ is an amino acid selected from the group consisting of D, N, S, T, K, A, Y, and L;
wherein Formula VIII is represented as:
(I₁)ₘ-(I₂)ₘ-(I₃)ₘ-(I₄)ₘ-(I₅)ₘ-(I₆)ₘ-(I₇)ₓ-(I₈)ₘ-(I₉)ₘ-(I₁₀)ₘ-(I₁₁)ₓ- (I₁₂)ₘ-(I₁₃)ₓ-(I₁₄)ₓ-(I₁₅)ₘ-(I₁₆)ₓ-(I₁₇)ₘ-I₁₈-I₁₉-I₂₀-I₂₁-I₂₂-I₂₃ (Formula VIII)
wherein:
each m is, independently, 0, 1, or 2; and
each x is, independently, 0, 1, 2, 3, or 4;
wherein:
each I₁ and I₆ is each, independently, an amino acid selected from the group consisting of S, Q, E, A, I, G, V, R, T, and Y;
each I₂ is, independently, an amino acid selected from the group consisting of T, S, E, R, P, V, I, and F;
each I₃ is, independently, L.
each I₄ is, independently, an amino acid selected from the group consisting of T, N, K, and M;
each I₅ is, independently, an amino acid selected from the group consisting of P, A, and D;
each I₇ is, independently, an amino acid selected from the group consisting of T, S, K, H, Y, V, and F;
each I₈ and I₁₅ is each, independently, an amino acid selected from the group consisting of F, L, W, A, T, M, Y, and C;
each I₉ is, independently, an amino acid selected from the group consisting of I, L, and V;
each I₁₀ and I₁₆ is each, independently, an amino acid selected from the group consisting of G, S, N, E, D, A, K, H, C, P, and F;
each I₁₁ is, independently, an amino acid selected from the group consisting of I, L, V, A, T, and S;
each I₁₂ is, independently, an amino acid selected from the group consisting of T, N, A, E, and G;
each I₁₃ is, independently, an amino acid selected from the group consisting of E, Q, S, T, R, K, A, L, D, and F;
each I₁₄ is, independently, an amino acid selected from the group consisting of T, S, Q, F, A, G, V, I, and L;
each I₁₇ is, independently, an amino acid selected from the group consisting of I, L, V, N, A, T, and S;
I₁₈ and I₂₁ are each, independently, an amino acid selected from the group consisting of R, K, Q, and A;
I₁₉ is an amino acid selected from the group consisting of H, R, S, N, T, A, V, and W;
I₂₀ is an amino acid selected from the group consisting of K, N, Q, D, E, A, and I;
I₂₂ is an amino acid selected from the group consisting of D, N, S, A, Y, and L; and
I₂₃ is an amino acid selected from the group consisting of V, I, L, F, and A;
wherein Formula X is represented as:
(J₁)_{z}-(J₂)_{z}-(J₃)_{z}-(J₄)_{z}-(J₅)_{z}-(J₆)_{z}-(J₇)_{z}-(J₈)_{z}-(J₉)_{z}-(J₁₀)_{z}-(J₁₁)_{z}- (J₁₂)_{z}-(J₁₃)_{z}-(J₁₄)_{z}-(J₁₅)_{z}-(J₁₆)_{z}-(J₁₇)_{z}-(J₁₈)_{z}-(J₁₉)_{z}-(J₂₀)_{z}-(J₂₁)_{z}-J₂₂-J₂₃-J₂₄-J₂₅ (Formula X)
wherein:
each z is, independently, 0, 1, 2, 3, 4, or 5;
wherein:
each J₁ is, independently, an amino acid selected from the group consisting of H, K, G, A, P, F, and L;
each J₂ is, independently, an amino acid selected from the group consisting of D, E, N, G, P, H, T, R, K, and A;
each J₃ is, independently, an amino acid selected from the group consisting of G, A, P, V, and L;
each J₄ is, independently, an amino acid selected from the group consisting of F, I, P, A, S, E, D, R, and K;
each J₅ is, independently, an amino acid selected from the group consisting of S, R, T, G, K, E, D, and C;
each J₆ is, independently, an amino acid selected from the group consisting of T, S, A, D, and F;
each J₇ is, independently, an amino acid selected from the group consisting of D, E, N, G, P, H, T, R, K, and A;
each J₈ is, independently, an amino acid selected from the group consisting of Y, C, A, W, I, S, E, D, F, L, R, and K;
each J₉ is, independently, an amino acid selected from the group consisting of H, K, N, D, G, T, A, C, Y, V, and L;
each J₁₀ is, independently, an amino acid selected from the group consisting of L, V, A, G, E, I, P, and R;
each J₁₁ is, independently, an amino acid selected from the group consisting of I, W, V, Y, P, T, N, S, R, and K;
each J₁₂ is, independently, an amino acid selected from the group consisting of A, G, Q, N, R, Y, E, D, and L;
each J₁₃ is, independently, an amino acid selected from the group consisting of I, L, W, V, M, Y, P, A, S, and G;
each J₁₄ is, independently, an amino acid selected from the group consisting of V, C, L, F, A, T, N, G, and R;
each J₁₅ is, independently, an amino acid selected from the group consisting of G, S, R, K, A, T, H, E, W, L, and F;
each J₁₆ is, independently, an amino acid selected from the group consisting of D, E, Q, S, H, T, R, G, Y, V, F, and L;
each J₁₇ is, independently, an amino acid selected from the group consisting of E, S, G, Y, I, and L;
each J₁₈ is, independently, an amino acid selected from the group consisting of A, S, P, H, and V;
each J₁₉ is, independently, an amino acid selected from the group consisting of N, E, R, K, and A;
each J₂₀ is, independently, an amino acid selected from the group consisting of R, T, V, I, and L;
each J₂₁ is, independently, an amino acid selected from the group consisting of L, V, A, G, E, I, P, and R;
J₂₂ is an amino acid selected from the group consisting of K, R, D, T, M, and W;
J₂₃ is an amino acid selected from the group consisting of R, T, V, I, and L;
J₂₄ is an amino acid selected from the group consisting of S, N, G, E, D, P, and W; and
J₂₅ is an amino acid selected from the group consisting of A, T, S, Y, M, V, and L;
wherein Formula XI is represented as:
(K₁)_{b}-(K₂)_{b}-(K₃)_{b}-(K₄)_{b}-(K₅)_{b}-(K₆)_{b}-(K₇)_{b}-(K₈)_{b}-(K₉)_{b}-(K₁₀)_{b}-(K₁₁)_{b}- (K₁₂)_{b}-(K₁₃)_{b}-(K₁₄)_{b}-(K₁₅)_{b}-(K₁₆)_{b}-(K₁₇)_{b}-(K₁₈)_{b}-(K₁₉)_{b}-(K₂₀)_{b}-(K₂₁)_{b}-(K₂₂)_{b}-(K₂₃)_{b}-(K₂₄)_{b}-(K₂₅)_{b}-(K₂₆)_{b}-(K₂₇)_{b}-(K₂₈)_{b}-(K₂₉)_{b}-(K₃₀)_{b}-(K₃₁)_{b}-(K₃₂)_{b}-(K₃₃)_{b}-(K₃₄)_{b}-(K₃₅)_{b}-(K₃₆)_{b}-(K₃₇)_{b}-(K₃₈)_{b}-(K₃₉)_{b}-(K₄₀)_{b}-(K₄₁)_{b}-(K₄₂)_{b}-(K₄₃)_{b}-(K₄₄)_{b}-(K₄₅)_{b}-(K₄₆)_{b}-(K₄₇)_{b}-(K₄₈)_{b}-(K₄₉)_{b}-(K₅₀)_{b}-(K₅₁)_{b}-(K₅₂)_{b}-(K₅₃)_{b}-(K₅₄)_{b}-(K₅₅)_{b}-(K₅₆)_{b}-(K₅₇)_{b}-(K₅₈)_{b}-(K₅₉)_{b}-(K₆₀)_{b}-(K₆₁)_{b}-(K₆₂)_{b}-(K₆₃)_{b}-(K₆₄)_{b}-(K₆₅)_{b}-(K₆₆)_{b}-(K₆₇)_{b}-(K₆₈)_{b}-(K₆₉)_{b}-(K₇₀)_{b}-(K₇₁)_{b}-(K₇₂)_{b}-(K₇₃)_{b}-(K₇₄)_{b}-(K₇₅)_{b}-(K₇₆)_{b}-(K₇₇)_{b}-(K₇₈)_{b}-(K₇₉)_{b}-(K₈₀)_{b}-(K₈₁)_{b}-(K₈₂)_{b}-(K₈₃)_{b}-(K₈₄)_{b}-(K₈₅)_{b}-(K₈₆)_{b}-(K₈₇)_{b}-(K₈₈)_{b}-K₈₉-K₈₉-K₈₉-K₈₉-K₈₉ (Formula XI)
wherein:
each b is, independently, 0, 1, 2, or 3;
wherein:
each K₁ is, independently, an amino acid selected from the group consisting of S, G, D, A, C, P, and Y;
each K₂ is, independently, an amino acid selected from the group consisting of Q, S, E, T, R, K, G, A, Y, M, V, and I;
each K₃ is, independently, an amino acid selected from the group consisting of G, S, N, T, Q, D, P, L, F, V, K, A, and C;
each K₄ is, independently, an amino acid selected from the group consisting of R, G, N, D, A, P, Y, and L;
each K₅ is, independently, an amino acid selected from the group consisting of E, A, V, Q, G, Y, M, I, and L;
each K₆ is, independently, an amino acid selected from the group consisting of S, Q, R, T, D, G, E, A, and K;
each K₇ is, independently, an amino acid selected from the group consisting of N, Q, R, H, K, A, I, F, and L;
each K₈ is, independently, an amino acid selected from the group consisting of A, T, Q, G, R, K, D, L, F, C, V, S, and H;
each K₉ is, independently, an amino acid selected from the group consisting of G, S, N, T, Q, D, P, L, F, V, K, A, and C;
each K₁₀ is, independently, an amino acid selected from the group consisting of K, H, E, A, Y, L, and F;
each K₁₁ is, independently, an amino acid selected from the group consisting of S, T, K, E, A, C, W, F, and L;
each K₁₂ is, independently, an amino acid selected from the group consisting of K, R, H, S, Q, D, E, and A;
each K₁₃ is, independently, an amino acid selected from the group consisting of G, S, T, E, P, W, R, N, and Q;
each K₁₄ is, independently, an amino acid selected from the group consisting of D, Q, S, G, V, E, N, H, R, P, and F;
each K₁₅ is, independently, an amino acid selected from the group consisting of C, A, M, V, S, E, G, I, F, and L;
each K₁₆ is, independently, an amino acid selected from the group consisting of R, K, S, Q, T, Y, N, V, I, L, and C;
each K₁₇ is, independently, an amino acid selected from the group consisting of A, G, S, Q, Y, E, D, H, and I;
each K₁₈ is, independently, an amino acid selected from the group consisting of R, K, S, Q, T, Y, N, V, I, L, and C;
each K₁₉ is, independently, an amino acid selected from the group consisting of E, D, T, H, K, G, P, V, and L;
each K₂₀ is, independently, an amino acid selected from the group consisting of F, L, I, V, M, T, G, and R;
each K₂₁ is, independently, an amino acid selected from the group consisting of E, D, S, G, A, C, and P;
each K₂₂ is, independently, an amino acid selected from the group consisting of D, T, G, A, Y, N, S, C, P, W, and I;
each K₂₃ is, independently, an amino acid selected from the group consisting of G, S, N, E, D, Y, and L;
each K₂₄ is, independently, an amino acid selected from the group consisting of T, S, E, G, P, and I;
each K₂₅ is, independently, an amino acid selected from the group consisting of K, S, G, T, and L;
each K₂₆ is, independently, an amino acid selected from the group consisting of S, G, K, E, D, P, and F;
each K₂₇ is, independently, an amino acid selected from the group consisting of P, A, E, L, T, Q, S, G, K, Y, F, C, V, W, and R;
each K₂₈ is, independently, an amino acid selected from the group consisting of E, D, Q, S, T, P, and L;
each K₂₉ is, independently, an amino acid selected from the group consisting of A, T, S, E, V, W, and I;
each K₃₀ is, independently, an amino acid selected from the group consisting of K, H, S, G, N, Q, P, and Y;
each K₃₁ is, independently, an amino acid selected from the group consisting of L, F, V, P, A, N, G, and H;
each K₃₂ is, independently, an amino acid selected from the group consisting of A, G, N, P, R, E, and K;
each K₃₃ is, independently, an amino acid selected from the group consisting of R, S, N, A, P, Y, V, I, F, and G;
each K₃₄ is, independently, an amino acid selected from the group consisting of E, S, T, V, I, H, A, P, F, and L;
each K₃₅ is, independently, an amino acid selected from the group consisting of A, T, Q, P, R, V, N, E, and L;
each K₃₆ is, independently, an amino acid selected from the group consisting of R, K, H, G, Q, D, T, Y, and F;
each K₃₇ is, independently, an amino acid selected from the group consisting of D, E, N, T, C, Y, V, I, and L;
each K₃₈ is, independently, an amino acid selected from the group consisting of S, Q, R, T, D, G, E, A, and K;
each K₃₉ is, independently, an amino acid selected from the group consisting of K, S, G, Q, D, E, A, M, I, and L;
each K₄₀ is, independently, an amino acid selected from the group consisting of H, K, S, D, E, T, P, and L;
each K₄₁ is, independently, an amino acid selected from the group consisting of A, T, S, N, P, V, L, and F;
each K₄₂ is, independently, an amino acid selected from the group consisting of K, D, M, V, I, L, and F;
each K₄₃ is, independently, an amino acid selected from the group consisting of G, S, N, T, Q, D, P, L, F, V, K, A, and C;
each K₄₄ is, independently, an amino acid selected from the group consisting of L, T, F, V, P, A, K, and I;
each K₄₅ is, independently, an amino acid selected from the group consisting of G, S, K, N, T, Q, D, A, P, L, F, and V;
each K₄₆ is, independently, an amino acid selected from the group consisting of L, F, Q, S, G, and D;
each K₄₇ is, independently, an amino acid selected from the group consisting of S, R, E, A, P, V, W, and L;
each K₄₈ is, independently, an amino acid selected from the group consisting of A, S, V, G, Q, R, E, D, L, T, K, F, C, and H;
each K₄₉ is, independently, an amino acid selected from the group consisting of E, S, T, R, G, A, P, and L;
each K₅₀ is, independently, an amino acid selected from the group consisting of S, N, R, A, P, and Y;
each K₅₁ is, independently, an amino acid selected from the group consisting of G, A, T, H, M, V, L, and F;
each K₅₂ is, independently, an amino acid selected from the group consisting of S, T, H, A, C, M, and L;
each K₅₃ is, independently, an amino acid selected from the group consisting of G, S, T, E, P, W, R, N, and Q;
each K₅₄ is, independently, an amino acid selected from the group consisting of S, H, Y, F, N, Q, R, T, G, and K;
each K₅₅ is, independently, an amino acid selected from the group consisting of A, T, Q, E, M, V, I, L, and F;
each K₅₆ is, independently, an amino acid selected from the group consisting of S, N, E, A, P, F, and L;
each K₅₇ is, independently, an amino acid selected from the group consisting of D, S, R, K, A, V, W, I, and F;
each K₅₈ is, independently, an amino acid selected from the group consisting of K, S, G, D, T, L, R, E, Y, and N;
each K₅₉ is, independently, an amino acid selected from the group consisting of S, R, G, A, V, and F;
each K₆₀ is, independently, an amino acid selected from the group consisting of A, T, Q, G, R, K, D, L, F, C, V, S, and H;
each K₆₁ is, independently, an amino acid selected from the group consisting of R, S, G, N, E, T, A, and V;
each K₆₂ is, independently, an amino acid selected from the group consisting of E, S, T, V, I, H, A, P, F, and L;
each K₆₃ is, independently, an amino acid selected from the group consisting of A, G, S, Q, R, E, D, V, L, T, K, F, C, and H;
each K₆₄ is, independently, an amino acid selected from the group consisting of E, A, V, Q, G, Y, M, I, and L;
each K₆₅ is, independently, an amino acid selected from the group consisting of G, S, T, E, P, W, R, N, and Q;
each K₆₆ is, independently, an amino acid selected from the group consisting of A, G, P, M, N, V, and S;
each K₆₇ is, independently, an amino acid selected from the group consisting of T, Q, E, N, S, A, Y, V, W, and F;
each K₆₈ is, independently, an amino acid selected from the group consisting of I, V, P, and A;
each K₆₉ is, independently, an amino acid selected from the group consisting of D, Q, S, G, V, E, N, H, R, P, and F;
each K₇₀ is, independently, an amino acid selected from the group consisting of G, S, R, N, T, Y, L, and F;
each K₇₁ is, independently, an amino acid selected from the group consisting of E, D, N, S, T, H, and Y;
each K₇₂ is, independently, an amino acid selected from the group consisting of L, I, W, V, A, T, S, E, R, and K;
each K₇₃ is, independently, an amino acid selected from the group consisting of G, S, K, A, C, F, N, T, Q, D, P, L, and V;
each K₇₄ is, independently, an amino acid selected from the group consisting of A, S, N, P, K, V, I, and L;
each K₇₅ is, independently, an amino acid selected from the group consisting of P, A, E, L, T, Q, S, G, K, Y, F, C, V, W, and R;
each K₇₆ is, independently, an amino acid selected from the group consisting of L, T, F, V, P, A, K, and I;
each K₇₇ is, independently, an amino acid selected from the group consisting of M, V, Y, L, A, N, E, and H;
each K₇₈ is, independently, an amino acid selected from the group consisting of D, T, G, A, Y, N, S, C, P, W, and I;
each K₇₉ is, independently, an amino acid selected from the group consisting of A, S, V, G, Q, R, E, D, L, T, K, F, C, and H;
each K₈₀ is, independently, an amino acid selected from the group consisting of K, R, S, A, P, V, I, and L;
each K₈₁ is, independently, an amino acid selected from the group consisting of F, L, V, A, T, S, E, D, R, and K;
each K₈₂ is, independently, an amino acid selected from the group consisting of L, F, M, A, N, G, and E;
each K₈₃ is, independently, an amino acid selected from the group consisting of D, S, H, A, V, I, F, and L;
each K₈₄ is, independently, an amino acid selected from the group consisting of A, T, Q, S, R, V, L, G, H, F, K, D, and C;
each K₈₅ is, independently, an amino acid selected from the group consisting of T, Q, E, N, S, A, Y, V, W, and F;
each K₈₆ is, independently, an amino acid selected from the group consisting of A, P, R, Y, K, D, M, L, and F;
each K₈₇ is, independently, an amino acid selected from the group consisting of N, S, D, T, A, P, and L;
each K₈₈ is, independently, an amino acid selected from the group consisting of R, S, N, A, P, Y, V, I, F, and G;
K₈₉ is an amino acid selected from the group consisting of K, R, H, G, E, T, Y, and I;
K₉₀ is an amino acid selected from the group consisting of R, S, G, N, Q, A, Y, and W;
K₉₁ is an amino acid selected from the group consisting of V, I, and F;
K₉₂ is an amino acid selected from the group consisting of A, G, P, M, N, V, and S; and
K₉₃ is an amino acid selected from the group consisting of E, D, Q, S, R, K, M, and L;
wherein Formula XIV is represented as:
(M₁)_{b}-(M₂)_{b}-(M₃)_{b}-(M₄)_{b}-(M₅)_{b}-(M₆)_{b}-(M₇)_{b}-(M₈)_{b}-(M₉)_{b}-(M₁₀)_{b}-(M₁₁)_{b}- (M₁₂)_{b}-(M₁₃)_{b}-(M₁₄)_{b}-(M₁₅)_{b}-(M₁₆)_{b}-(M₁₇)_{b}-(M₁₈)_{b}-(M₁₉)_{b}-(M₂₀)_{b}-(M₂₁)_{b}-(M₂₂)_{b}-(M₂₃)_{b}-(M₂₄)_{b}-(M₂₅)_{b}-(M₂₆)_{b}-(M₂₇)_{b}-(M₂₈)_{b}-(M₂₉)_{b}-(M₃₀)_{b}-(M₃₁)_{b}-(M₃₂)_{b}-(M₃₃)_{b}-(M₃₄)_{b}-(M₃₅)_{b}-(M₃₆)_{b}-(M₃₇)_{b}-(M₃₈)_{b}-(M₃₉)_{b}-(M₄₀)_{b}-(M₄₁)_{b}-(M₄₂)_{b}-(M₄₃)_{b}-(M₄₄)_{b}-(M₄₅)_{b}-(M₄₆)_{b}-(M₄₇)_{b}-(M₄₈)_{b}-(M₄₉)_{b}-(M₅₀)_{b}-(M₅₁)_{b}-(M₅₂)_{b}-(M₅₃)_{b}-(M₅₄)_{b}-(M₅₅)_{b}-(M₅₆)_{b}-(M₅₇)_{b}-(M₅₈)_{b}-(M₅₉)_{b}-(M₆₀)_{b}-(M₆₁)_{b}-(M₆₂)_{b}-(M₆₃)_{b}-(M₆₄)_{b}-(M₆₅)_{b}-(M₆₆)_{b}-(M₆₇)_{c}-(M₆₈)_{c}-(M₆₉)_{c}-(M₇₀), (Formula XIV);
wherein:
each b is, independently, 0, 1, 2, or 3; and
each c is, independently, 1 or 2;
wherein:
each M₁ is, independently, an amino acid selected from the group consisting of A, T, C, S, Y, E, H, V, W, I, L, F, G, Q, N, P, R, K, D, and M;
each M₂ is, independently, an amino acid selected from the group consisting of S, T, A, N, R, G, E, P, V, F, L, Q, K, H, D, I, C, Y, M, and W;
each M₃ is, independently, an amino acid selected from the group consisting of G, S, R, A, T, Q, E, D, C, Y, V, I, L, and N;
each M₄ is, independently, an amino acid selected from the group consisting of R, H, N, Q, E, A, Y, M, V, W, F, and L;
each M₅ is, independently, an amino acid selected from the group consisting of P, Y, A, T, Q, S, G, D, R, K, C, V, I, L, and H;
each M₆ is, independently, an amino acid selected from the group consisting of T, Q, N, S, A, E, G, D, H, P, F, L, C, K, V, R, Y, I, M, and W;
each M₇ is, independently, an amino acid selected from the group consisting of A, G, S, Q, N, K, D, T, C, Y, E, H, V, W, I, L, F, P, R, and M;
each M₈ is, independently, an amino acid selected from the group consisting of T, Q, N, S, A, G, C, R, K, P, Y, M, V, I, L, F, E, W, D, and H;
each M₉ is, independently, an amino acid selected from the group consisting of G, S, H, P, R, A, T, Q, E, D, C, Y, V, I, L, N, W, F, K, and M;
each M₁₀ is, independently, an amino acid selected from the group consisting of Q, E, and W;
each M₁₁ is, independently, an amino acid selected from the group consisting of V, I, L, F, C, A, and T;
each M₁₂ is, independently, an amino acid selected from the group consisting of S, G, A, N, Q, R, T, K, E, H, D, P, I, F, V, C, Y, L, M, and W;
each M₁₃ is, independently, an amino acid selected from the group consisting of T, Q, N, S, D, P, F, A, E, G, H, L, C, K, V, R, Y, I, M, and W;
each M₁₄ is, independently, an amino acid selected from the group consisting of L, F, I, V, M, Y, A, T, Q, N, S, D, K, P, E, R, H, G, and C;
each M₁₅ is, independently, an amino acid selected from the group consisting of S, P, V, E, T, A, F, L, N, R, G, Q, K, H, D, I, C, Y, M, and W;
each M₁₆ is, independently, an amino acid selected from the group consisting of T, S, A, E, G, C, R, P, Y, M, V, W, I, F, L, Q, N, D, H, and K;
each M₁₇ is, independently, an amino acid selected from the group consisting of D, E, Q, T, K, P, F, N, S, G, A, Y, R, and V;
each M₁₈ is, independently, an amino acid selected from the group consisting of G, S, H, P, R, D, N, A, T, Q, E, C, Y, V, I, L, W, F, K, and M;
each M₁₉ is, independently, an amino acid selected from the group consisting of T, P, F, S, A, E, G, C, R, Y, M, V, W, I, L, Q, N, D, H, and K;
each M₂₀ is, independently, an amino acid selected from the group consisting of L, F, I, V, Y, A, T, Q, S, D, M, N, K, P, E, R, H, G, and C;
each M₂₁ is, independently, an amino acid selected from the group consisting of F, L, W, Y, and P;
each M₂₂ is, independently, an amino acid selected from the group consisting of P, K, Y, A, T, Q, S, G, D, R, C, V, I, L, and H;
each M₂₃ is, independently, an amino acid selected from the group consisting of T, P, F, S, A, E, G, C, R, Y, M, V, W, I, L, Q, N, D, H, and K;
each M₂₄ is, independently, an amino acid selected from the group consisting of S, T, A, N, R, G, E, P, V, F, L, Q, K, H, D, I, C, Y, M, and W;
each M₂₅ is, independently, an amino acid selected from the group consisting of F, W, Y, and P;
each M₂₆ is, independently, an amino acid selected from the group consisting of T, P, F, Q, N, S, A, E, G, D, K, Y, C, V, I, L, and H;
each M₂₇ is, independently, an amino acid selected from the group consisting of D, E, Q, N, S, T, R, K, G, A, Y, P, V, and F;
each M₂₈ is, independently, an amino acid selected from the group consisting of T, Q, N, S, A, G, C, R, K, P, Y, M, V, I, L, F, E, W, D, and H;
each M₂₉ is, independently, an amino acid selected from the group consisting of S, T, E, A, P, V, F, L, N, R, G, Q, K, H, D, I, C, Y, M, and W;
each M₃₀ is, independently, an amino acid selected from the group consisting of D, Q, N, H, K, G, C, and Y;
each M₃₁ is, independently, an amino acid selected from the group consisting of F, L, W, Y, and P;
each M₃₂ is, independently, an amino acid selected from the group consisting of S, T, E, A, P, V, F, L, N, R, G, Q, K, H, D, I, C, Y, M, and W;
each M₃₃ is, independently, an amino acid selected from the group consisting of A, G, S, Q, N, K, D, T, C, Y, E, H, V, W, I, L, F, P, R, and M;
each M₃₄ is, independently, an amino acid selected from the group consisting of T, A, V, I, P, F, Q, N, S, E, G, D, K, Y, C, L, and H;
each M₃₅ is, independently, an amino acid selected from the group consisting of G, S, R, N, H, D, P, A, T, Q, E, C, Y, V, I, L, W, F, K, and M;
each M₃₆ is, independently, an amino acid selected from the group consisting of T, Q, S, A, E, D, K, H, P, Y, V, W, I, F, L, N, G, and C;
each M₃₇ is, independently, an amino acid selected from the group consisting of I, L, W, V, and M;
each M₃₈ is, independently, an amino acid selected from the group consisting of A, G, S, Q, N, K, D, C, P, R, Y, E, V, W, T, H, M, and F;
each M₃₉ is, independently, an amino acid selected from the group consisting of S, T, E, P, V, A, F, L, N, R, G, Q, K, H, D, I, C, Y, M, and W;
each M₄₀ is, independently, an amino acid selected from the group consisting of T, S, A, D, P, M, Q, E, K, H, Y, V, W, I, F, L, N, G, and C;
each M₄₁ is, independently, an amino acid selected from the group consisting of L, F, I, V, Y, A, T, Q, S, D, M, N, K, P, E, R, H, G, and C;
each M₄₂ is, independently, an amino acid selected from the group consisting of P, Y, A, T, Q, S, N, W, G, I, E, D, L, K, and H;
each M₄₃ is, independently, an amino acid selected from the group consisting of S, E, P, V, T, A, F, L, N, R, G, Q, K, H, D, I, C, Y, M, and W;
each M₄₄ is, independently, an amino acid selected from the group consisting of N, Q, S, E, D, T, H, K, G, A, P, W, and F;
each M₄₅ is, independently, an amino acid selected from the group consisting of V, I, L, F, C, A, and T;
each M₄₆ is, independently, an amino acid selected from the group consisting of A, T, S, N, R, Y, K, D, H, M, L, F, G, Q, C, P, E, V, and W;
each M₄₇ is, independently, an amino acid selected from the group consisting of I, L, and V;
each M₄₈ is, independently, an amino acid selected from the group consisting of S, P, V, E, T, A, F, L, N, R, G, Q, K, H, D, I, C, Y, M, and W;
each M₄₉ is, independently, an amino acid selected from the group consisting of F, V, A, T, Q, N, S, E, G, D, and H;
each M₅₀ is, independently, an amino acid selected from the group consisting of L, F, I, V, Y, A, T, Q, S, D, M, N, K, P, E, R, H, G, and C;
each M₅₁ is, independently, an amino acid selected from the group consisting of G, S, R, H, D, P, N, A, T, Q, E, C, Y, V, I, L, W, F, K, and M;
each M₅₂ is, independently, an amino acid selected from the group consisting of T, N, S, G, C, R, H, A, D, P, M, Q, E, K, Y, V, W, I, F, and L;
each M₅₃ is, independently, an amino acid selected from the group consisting of I, L, W, V, and M;
each M₅₄ is, independently, an amino acid selected from the group consisting of P, K, Y, A, T, Q, S, G, D, R, C, V, I, L, and H;
each M₅₅ is, independently, an amino acid selected from the group consisting of D, E, Q, N, S, K, G, A, Y, P, F, T, R, and V;
each M₅₆ is, independently, an amino acid selected from the group consisting of L, F, I, V, Y, P, A, T, Q, N, S, G, E, D, K, H, M, C, and R;
each M₅₇ is, independently, an amino acid selected from the group consisting of S, P, V, E, T, A, F, L, N, R, G, Q, K, H, D, I, C, Y, M, and W;
each M₅₈ is, independently, an amino acid selected from the group consisting of P, M, V, I, L, and F;
each M₅₉ is, independently, an amino acid selected from the group consisting of N, Q, S, E, D, T, R, K, G, A, and Y;
each M₆₀ is, independently, an amino acid selected from the group consisting of G, S, H, P, R, D, N, A, T, Q, E, C, Y, V, I, L, W, F, K, and M;
each M₆₁ is, independently, an amino acid selected from the group consisting of S, P, V, T, A, R, K, E, H, C, Y, I, F, L, N, Q, G, D, M, and W;
each M₆₂ is, independently, an amino acid selected from the group consisting of P, K, A, Y, T, Q, S, G, D, R, C, V, I, L, and H;
each M₆₃ is, independently, an amino acid selected from the group consisting of A, G, S, N, E, K, D, H, M, V, W, I, L, F, T, R, Y, Q, C, and P;
each M₆₄ is, independently, an amino acid selected from the group consisting of D, E, Q, T, K, P, F, N, S, G, A, Y, R, and V;
each M₆₅ is, independently, an amino acid selected from the group consisting of L, V, F, I, Y, P, A, T, Q, N, S, G, E, D, K, H, M, C, and R;
each M₆₆ is, independently, an amino acid selected from the group consisting of S, N, R, T, G, K, E, H, D, A, P, V, C, Y, I, F, L, Q, M, and W;
each M₆₇ is, independently, an amino acid selected from the group consisting of K, R, H, S, G, N, Q, D, E, T, A, C, P, Y, M, V, W, I, L, and F;
each M₆₈ is, independently, an amino acid selected from the group consisting of R, K, H, S, G, N, Q, D, E, T, A, C, P, Y, M, V, W, I, L, and F;
each M₆₉ is, independently, an amino acid selected from the group consisting of S, A, N, Q, R, T, G, K, E, H, D, A, C, P, Y, M, V, W, I, F, and L; and
each M₇₀ is, independently, an amino acid selected from the group consisting of T, Q, N, S, A, E, G, D, C, R, K, H, P, Y, M, V, W, I, F, and L; and
wherein Formula XV is represented as:
(N₁)_{b}-(N₂)_{b}-(N₃)_{b}-(N₄)_{b}-(N₅)_{b}-(N₆)_{b}-(N₇)_{b}-(N₈)_{b}-(N₉)_{b}-(N₁₀)_{b}-(N₁₁)_{b}- (N₁₂)_{b}-(N₁₃)_{b}-(N₁₄)_{b}-(N₁₅)_{b}-(N₁₆)_{b}-(N₁₇)_{b}-(N₁₈)_{b}-(N₁₉)_{b}-(N₂₀)_{b}-(N₂₁)_{b}-(N₂₂)_{b}-(N₂₃)_{b}-(N₂₄)_{b}-(N₂₅)_{b}-(N₂₆)_{b}-(N₂₇)_{b}-(N₂₈)_{b}-(N₂₉)_{b}-(N₃₀)_{b}-(N₃₁)_{b}-(N₃₂)_{b}-(N₃₃)_{b}-(N₃₄)_{b}-(N₃₅)_{b}-(N₃₆)_{b}-(N₃₇)_{b}-(N₃₈)_{b}-(N₃₉)_{b}-(N₄₀)_{b}-(N₄₁)_{b}-(N₄₂)_{b}-(N₄₃)_{b}-(N₄₄)_{b}-(N₄₅)_{b}-(N₄₆)_{b}-(N₄₇)_{b}-(N₄₈)_{b}-(N₄₉)_{b}-(N₅₀)_{b}-(N₅₁)_{b}-(N₅₂)_{b}-(N₅₃)_{b}-(N₅₄)_{b}-(N₅₅)_{b}-(N₅₆)_{b}-(N₅₇)_{b}-(N₅₈)_{b}-(N₅₉)_{b}-(N₆₀)_{b}-(N₆₁)_{b}-(N₆₂)_{b}-(N₆₃)_{b}-(N₆₄)_{b}-(N₆₅)_{b}-(N₆₆)_{b}-(N₆₇)_{c}-(N₆₈)ᵣ-(N₆₉)ᵣ-(N₇₀)ᵣ-(N₇₁)_{c} (Formula XV);
wherein:
each b is, independently, 0, 1, 2, or 3; and
each c is, independently, 1 or 2;
wherein:
each N₁ is, independently, an amino acid selected from the group consisting of S, N, D, Q, R, T, G, E, H, A, P, M, V, K, Y, W, F, L, I, and C;
each N₂ is, independently, an amino acid selected from the group consisting of P, A, S, Y, V, T, G, I, E, and C;
each N₃ is, independently, an amino acid selected from the group consisting of T, S, G, D, C, A, L, N, R, P, Y, V, W, I, and F;
each N₄ is, independently, an amino acid selected from the group consisting of S, R, E, A, Q, K, N, D, T, G, H, C, P, Y, I, F, L, M, V, and W;
each N₅ is, independently, an amino acid selected from the group consisting of T, Q, N, G, C, M, S, A, E, D, Y, V, I, F, L, and W;
each N₆ is, independently, an amino acid selected from the group consisting of I, V, L, F, W, Y, A, T, S, E, D, and H;
each N₇ is, independently, an amino acid selected from the group consisting of P, V, A, S, N, G, E, L, and K;
each N₈ is, independently, an amino acid selected from the group consisting of A, G, Q, T, S, N, P, R, D, V, K, C, Y, W, I, L, and F;
each N₉ is, independently, an amino acid selected from the group consisting of F, Y, A, T, N, and R;
each N₁₀ is, independently, an amino acid selected from the group consisting of T, Q, N, R, K, M, S, E, D, H, P, V, W, I, F, and L;
each N₁₁ is, independently, an amino acid selected from the group consisting of A, G, Q, T, S, N, P, R, D, V, K, C, Y, W, I, L, and F;
each N₁₂ is, independently, an amino acid selected from the group consisting of S, N, Q, R, T, G, K, E, H, D, A, P, L, M, V, Y, W, F, I, and C;
each N₁₃ is, independently, an amino acid selected from the group consisting of L, F, I, W, V, M, Y, C, A, T, Q, N, S, G, E, D, and R;
each N₁₄ is, independently, an amino acid selected from the group consisting of V, I, L, A, T, S, G, R, P, Y, N, H, C, M, F, Q, E, K, and D;
each N₁₅ is, independently, an amino acid selected from the group consisting of S, N, Q, T, G, K, E, H, D, A, C, P, Y, I, F, L, R, M, V, and W;
each N₁₆ is, independently, an amino acid selected from the group consisting of T, N, S, A, D, R, P, Y, V, W, I, F, and L;
each N₁₇ is, independently, an amino acid selected from the group consisting of S, N, Q, R, K, E, D, A, T, G, H, C, P, Y, I, F, L, M, V, and W;
each N₁₈ is, independently, an amino acid selected from the group consisting of V, A, T, S, G, R, W, I, C, L, F, E, D, K, P, Y, N, H, M, and Q;
each N₁₉ is, independently, an amino acid selected from the group consisting of T, Q, N, S, A, E, G, D, Y, M, V, I, F, L, and W;
each N₂₀ is, independently, an amino acid selected from the group consisting of S, Q, R, K, E, A, N, D, T, G, H, C, P, Y, I, F, L, M, V, and W;
each N₂₁ is, independently, an amino acid selected from the group consisting of V, W, I, C, L, F, A, T, S, E, D, K, G, R, P, Y, N, H, M, and Q;
each N₂₂ is, independently, an amino acid selected from the group consisting of T, Q, N, S, A, D, C, K, P, Y, M, V, W, I, F, G, E, H, R, and L;
each N₂₃ is, independently, an amino acid selected from the group consisting of L, F, I, V, P, A, T, Q, S, G, R, K, H, M, Y, and D;
each N₂₄ is, independently, an amino acid selected from the group consisting of T, Q, S, A, G, P, Y, I, K, H, V, F, L, N, D, C, M, W, E, and R;
each N₂₅ is, independently, an amino acid selected from the group consisting of S, R, E, A, Q, K, N, D, T, G, H, C, P, Y, I, F, L, M, V, and W;
each N₂₆ is, independently, an amino acid selected from the group consisting of T, N, D, S, A, R, P, Y, V, W, I, F, and L;
each N₂₇ is, independently, an amino acid selected from the group consisting of D, N, R, E, Q, S, H, T, K, G, W, I, P, and Y;
each N₂₈ is, independently, an amino acid selected from the group consisting of V, A, T, S, G, R, W, I, C, L, F, E, D, K, P, Y, N, H, M, and Q;
each N₂₉ is, independently, an amino acid selected from the group consisting of T, S, A, D, C, L, N, R, P, Y, V, W, I, and F;
each N₃₀ is, independently, an amino acid selected from the group consisting of P, Y, V, A, T, S, G, I, E, and C;
each N₃₁ is, independently, an amino acid selected from the group consisting of T, Q, S, A, G, K, H, P, Y, V, I, F, L, N, D, C, M, W, E, and R;
each N₃₂ is, independently, an amino acid selected from the group consisting of S, R, E, A, Q, K, N, D, T, G, H, C, P, Y, I, F, L, M, V, and W;
each N₃₃ is, independently, an amino acid selected from the group consisting of E, D, Q, N, S, T, H, R, G, A, P, F, and L;
each N₃₄ is, independently, an amino acid selected from the group consisting of D, N, R, E, Q, S, H, T, K, G, W, I, P, and Y;
each N₃₅ is, independently, an amino acid selected from the group consisting of T, Q, S, A, G, P, Y, I, K, H, V, F, L, N, D, C, M, W, E, and R;
each N₃₆ is, independently, an amino acid selected from the group consisting of G, S, K, A, T, Q, D, C, P, Y, V, W, I, L, and F;
each N₃₇ is, independently, an amino acid selected from the group consisting of F, Y, A, T, N, and R;
each N₃₈ is, independently, an amino acid selected from the group consisting of V, A, T, S, G, R, W, I, C, L, F, E, D, K, P, Y, N, H, M and Q;
each N₃₉ is, independently, an amino acid selected from the group consisting of L, F, I, W, V, M, C, A, T, Q, N, S, G, D, R, K, and H;
each N₄₀ is, independently, an amino acid selected from the group consisting of P, A, S, Y, V, T, G, I, E, and C;
each N₄₁ is, independently, an amino acid selected from the group consisting of D, N, R, G, Y, E, Q, S, H, T, K, W, and I;
each N₄₂ is, independently, an amino acid selected from the group consisting of S, R, E, A, N, T, G, P, V, Q, K, H, D, Y, M, I, F, L, C, and W;
each N₄₃ is, independently, an amino acid selected from the group consisting of G, S, R, K, A, N, Q, H, E, D, P, W, L, and F;
each N₄₄ is, independently, an amino acid selected from the group consisting of T, Q, S, A, G, P, Y, I, N, E, D, C, K, H, R, V, L, M, F, and W;
each N₄₅ is, independently, an amino acid selected from the group consisting of S, T, G, A, V, I, R, E, N, P, Q, K, H, D, Y, M, F, L, C, and W;
each N₄₆ is, independently, C;
each N₄₇ is, independently, an amino acid selected from the group consisting of S, N, R, T, G, K, E, H, D, A, P, Y, V, W, I, L, Q, M, F, and C;
each N₄₈ is, independently, an amino acid selected from the group consisting of G, S, R, K, N, T, Q, H, E, D, P, I, and L;
each N₄₉ is, independently, an amino acid selected from the group consisting of T, S, G, D, C, A, L, N, R, P, Y, V, W, I, and F;
each N₅₀ is, independently, an amino acid selected from the group consisting of V, A, T, S, G, I, R, P, Y, L, N, H, C, M, F, Q, E, and K;
each N₅₁ is, independently, an amino acid selected from the group consisting of A, T, G, S, Q, N, R, Y, E, H, M, V, W, I, L, and F;
each N₅₂ is, independently, an amino acid selected from the group consisting of D, E, Q, N, S, T, K, A, Y, P, M, W, I, F, and L;
each N₅₃ is, independently, an amino acid selected from the group consisting of A, T, C, G, S, N, P, R, K, D, H, M, and F;
each N₅₄ is, independently, an amino acid selected from the group consisting of L, F, I, V, P, A, T, Q, S, G, R, K, H, M, Y, and D;
each N₅₅ is, independently, an amino acid selected from the group consisting of E, D, N, T, R, K, G, A, and V;
each N₅₆ is, independently, an amino acid selected from the group consisting of A, G, Q, T, S, N, P, R, D, V, W, K, C, Y, I, L, and F;
each N₅₇ is, independently, an amino acid selected from the group consisting of Y, C, N, I, F, and L;
each N₅₈ is, independently, an amino acid selected from the group consisting of S, T, G, H, A, P, Y, V, F, L, N, R, K, E, D, W, I, Q, M, and C;
each N₅₉ is, independently, an amino acid selected from the group consisting of I, V, and L;
each N₆₀ is, independently, S each N₆₁ is, independently, an amino acid selected from the group consisting of G, S, R, K, A, N, T, Q, E, D, P, and Y;
each N₆₂ is, independently, an amino acid selected from the group consisting of I, V, L, F, W, Y, A, T, S, E, D, and H;
each N₆₃ is, independently, an amino acid selected from the group consisting of T, Q, N, G, C, M, S, A, E, D, Y, V, I, F, L, and W;
each N₆₄ is, independently, an amino acid selected from the group consisting of S, N, Q, R, G, K, E, D, P, Y, W, F, T, H, A, V, L, I, M, and C;
each N₆₅ is, independently, an amino acid selected from the group consisting of A, C, G, S, Q, N, R, Y, E, K, D, H, M, V, I, and L;
each N₆₆ is, independently, an amino acid selected from the group consisting of V, I, A, T, S, G, R, P, Y, L, N, H, C, M, F, Q, E, K, and D;
each N₆₇ is, independently, an amino acid selected from the group consisting of S, N, Q, R, T, G, K, E, H, D, A, C, P, Y, M, V, W, I, F, and L;
each N₆₈ is, independently, an amino acid selected from the group consisting of K, R, H, S, G, N, Q, D, E, T, A, C, P, Y, M, V, W, I, L, and F;
each N₆₉ is, independently, an amino acid selected from the group consisting of K, R, H, S, G, N, Q, D, E, T, A, C, P, Y, M, V, W, I, L, and F;
each N₇₀ is, independently, an amino acid selected from the group consisting of D, E, Q, N, S, H, T, R, K, G, A, C, Y, P, M, V, W, I, F, and L;
each N₇₁ is, independently, an amino acid selected from the group consisting of A, T, C, G, S, Q, N, P, R, Y, E, K, D, H, M, V, W, I, L, and F.

13. The pro-protein signal peptide of claim 12, wherein the signal peptide comprises an amino acid sequence having at least 90%, 91%, 92%, 93%, 94%, 95%, 96%, 97%, 98% or 99% identity to an amino acid sequence selected from the group consisting of SEQ ID NO. 17, 18, 19, 20, 21, 22, 23, 24, 25, 27, 29, 34, 35, 36, 37, 38, 56, 57, 58, 74, or 75.

14. The pro-protein signal peptide of claim 12, wherein the amino acid sequence is selected from the group consisting of SEQ ID NO. 17, 18, 19, 20, 21, 22, 23, 24, 25, 27, 29, 34, 35, 36, 37, 38, 56, 57, 58, 74, or 75.

15. A pro-protein signal peptide comprising an amino acid sequence having at least 90%, 91%, 92%, 93%, 94%, 95%, 96%, 97%, 98%, 99% or 100% identity to an amino acid sequence selected from the group consisting of SEQ ID NO. 17, 18, 19, 20, 21, 22, 23, 24, 25, 27, 29, 34, 35, 36, 37, 38, 56, 57, 58, 74, or 75.

16. A polypeptide comprising a formula of (X₁)ₙ(Y₁)ₘ-Z₁ wherein:
X₁ is a pre-protein signal peptide,
Y₁ is a pro-protein signal peptide, and
Z₁ is a payload protein,
wherein n is 0-1 and m is 0-1,
wherein n and m cannot concurrently be 0.

17. The polypeptide of claim 16, wherein n is 1 and X₁ comprises an amino acid sequence selected from the group consisting of Formula I, Formula II, Formula III, Formula IV, Formula V, Formula IX, and Formula XIII;
wherein Formula I is represented as:
A₁-(A₂)_{w}-A₃-(A₄)ₓ-(A₅)_{y}-A₆-A₇-A₈-A₉-A₁₀-(A₁₁)_{z} (Formula I)
wherein:
w and x are each, independently, 1, 2, 3, 4, or 5;
y is 2, 3, 4, 5, 6, 7, 8, 9, 10, 11, 12, 13, 14, 15, 16, 17, 18, 19, or 20; and
z is 1, 2, or 3;
wherein:
A₁ is methionine;
each A₂ is, independently, a neutral or positively-charged amino acid with a hydropathy index of less than about 1;
each A₃, A₅, A₈, and A₁₀ is each, independently, an amino acid with a hydropathy index greater than −1, excluding W and C;
each A₄ is, independently, a basic or neutral amino acid, excluding P, W, M, and C;
A₆ is an amino acid with a hydropathy index greater than −1, excluding W, M, and C;
A₇ is a non-aromatic amino acid with a hydropathy index of less than about 1.9 and an isoelectric point of about 5.4 to about 7.5, excluding P;
A₉ is an amino acid with a hydropathy index of greater than about −1.3; and
each A₁₁ is, independently, a neutral amino acid with a molecular weight of less than about 133 g/mol;
wherein Formula II is given by:
B₁-(B₂)ᵤ-(B₃)ᵥ-(B₄)_{w}-(B₅)ₓ-(B₆)_{y}-B₇-B₈-B₉-B₁₀-(B₁₁)_{z} (Formula II)
wherein:
u and w are each, independently, 0, 1, 2, or 3;
v and z are each, independently, 1, 2, or 3;
x is 0, 1, or 2; and
y is 2, 3, 4, 5, 6, 7, 8, 9, 10, 11, 12, 13, 14, 15, 16, 17, 18, 19, or 20;
wherein:
B₁ is methionine;
each B₂, B₄, B₆, B₈ and B₁₀ is each, independently, an amino acid with a hydropathy index of greater than about −1, excluding W and C;
each B₃ is, independently, a positively-charged or polar amino acid with a hydropathy index of less than about 1;
each B₅ is, independently, a polar amino acid with a hydropathy index of greater than about −5 and less than about −0.5, or an amino acid with an isoelectric point of about 5 to about 11, excluding P, W, M, and C;
each B₇ and B₁₁ is each, independently, a neutral amino acid with a molecular weight of less than about 133 g/mol; and
B₉ is an amino acid with a hydropathy index of greater than about −1.3;
wherein Formula III is represented as:
C₁-(C₂)ᵣ-(C₃)ₜ-(C₄)ᵤ-[(C₅)ᵥ-(C₆)_{w}]ₓ-(C₇)_{y}-(C₈)_{z}-C₉-C₁₀-C₁₁-[C₁₂-C₁₃]ₐ (Formula III)
wherein:
r is 1, 2, or 3;
t, u, y, and z are each, independently, 0, 1, 2, or 3;
v and w are each, independently, 0, 1, or 2;
a is 0 or 1; and
x is 2, 3, 4, 5, 6, 7, 8, 9, or 10;
wherein:
C₁ is methionine;
each C₂ is, independently, an amino acid having an isoelectric point of about 5.6 to about 10.8, a molecular weight of about 105 g/mol to about 175 g/mol, a hydropathy index of about −5.1 to about 0.6, and a helicity of about 0.8 to about 1;
each C₃, C₅, C₈, and C₁₀ is each, independently, an amino acid having an isoelectric point of about 2.75 to about 10.8, a molecular weight of about 75 g/mol to about 205 g/mol, a hydropathy index of about −5.1 to about 34, and a helicity of about 0.5 to about 1.3;
each C₄ and C₇ is each, independently, an amino acid having an isoelectric point of about 5 to about 10.8, a molecular weight of about 75 g/mol to about 205 g/mol, a hydropathy index of about −5.1 to about 34, and a helicity of about 0.5 to about 1.3;
each C₆, C₉, C₁₁, and C₁₂ is each, independently, an amino acid having an isoelectric point of about 2.75 to about 9.8, a molecular weight of about 75 g/mol to about 205 g/mol; a hydropathy index of about −4 to about 34, and a helicity of about 0.5 to about 1.3; and
C₁₃ is an amino acid having an isoelectric point of about 5.6 to about 6.3, a molecular weight of about 105 g/mol to about 120 g/mol, a hydropathy index of about 0 to about 9.4, and a helicity of about 0.5 to about 1.1;
wherein Formula IV is represented as:
D₁-(D₂)_{q}-(D₃)ᵣ-(D₄)ₜ-(D₈)ᵤ-[(D₆)ᵥ-(D₇)ₓ-(D₈)_{w}-(D₉)_{y}]_{z}-D₁₀-D₁₁-D₁₂-[D₁₃-D₁₄]ₐ (Formula IV)
wherein:
q is 1, 2, or 3;
r, t, and u are each, independently, 0, 1, 2, or 3;
v, w, x, and y are each, independently, 0, 1, or 2;
a is 0 or 1; and
z is 2, 3, 4, 5, 6, 7, 8, 9, or 10;
wherein:
D₁ is methionine;
each D₂ is, independently, an amino acid having an isoelectric point of about 2.7 to about 10.8, a molecular weight of about 75 g/mol to about 205 g/mol, a hydropathy index of about −5.1 to about 34, and a helicity of about 0.5 to about 1.3;
each D₃ is, independently, an amino acid having an isoelectric point of about 5 to about 10.8, a molecular weight of about 89 g/mol to about 205 g/mol, a hydropathy index of about −4 to about 34, and a helicity of about 0.5 to about 1.3;
each D₄, D₉ and D₁₁ is each, independently an amino acid having an isoelectric point of about 2.7 to about 10.8, a molecular weight of about 75 g/mol to about 205 g/mol, a hydropathy index of about −5.1 to about 34, and a helicity of about 0.5 to about 1.3;
each D₅ is, independently, an amino acid having an isoelectric point of about 3.2 to about 10.8, a molecular weight of about 75 g/mol to about 205 g/mol, a hydropathy index of about −5.1 to about 34, and a helicity of about 0.75 to about 1.3;
each D₆ is, independently, an amino acid having an isoelectric point from about 5 to about 10.8, a molecular weight of about 75 g/mol to about 205 g/mol, a hydropathy index of about −5.1 to about 34, and a helicity of about 0.5 to about 1.3;
each D₇ is, independently, an amino acid having an isoelectric point of about 5.4 to about 6.1, a molecular weight of about 117 g/mol to about 205 g/mol, a hydropathy index of about 2.5 to about 34, and a helicity of about 1 to about 1.3;
each D₈, D₁₀, D₁₂, and D₁₃ is each, independently, an amino acid having an isoelectric point of about 2.7 to about 10.8, a molecular weight of about 75 g/mol to about 182 g/mol, a hydropathy index of about −5.1 to about 32, and a helicity of about 0.75 to about 1.3; and
D₁₄ is an amino acid with an isoelectric point of about 2.7 to about 10.8, a molecular weight of about 75 g/mol to about 182 g/mol, a hydropathy index of about −5.1 to about 32, and a helicity of about 0.5 to about 1.3;
wherein Formula V is represented as:
E₁-[(E₂)ᵢ-(E₃)ⱼ-(E₄)_{q}]ᵣ-(E₅)-(E₆)ᵤ-(E₇)ᵥ-[(E₈)_{w}-(E₉)ₓ]_{y}-(E₁₀)_{z}-E₁₁- E₁₂-E₁₃-[E₁₄-E₁₅]ₐ (Formula V)
wherein:
i, j, q, w, x and a are each, independently, 0 or 1;
r is 1, 2, or 3;
t, u, v, and z are each, independently, 0, 1, 2, or 3; and
y is 2, 3, 4, 5, 6, 7, 8, 9, or 10;
wherein:
E₁ is methionine;
each E₂ is, independently, an amino acid having an isoelectric point of about 3.2 to about 10.8, a molecular weight of about 105 g/mol to about 175 g/mol, a hydropathy index of about −4 to about 1, and a helicity of about 0.85 to about 1;
each E₃ is, independently, an amino acid having an isoelectric point of about 2.7 to about 10.8, a molecular weight of about 75.1 g/mol to about 205 g/mol, a hydropathy index of about −5.1 to about 33.5, and a helicity of about 0.57 to about 1.3;
each E₄ is, independently, an amino acid having an isoelectric point of about 5 to about 10.8, a molecular weight of about 105 g/mol to about 205 g/mol, a hydropathy index of about −5.1 to about 33.5, and a helicity of about 0.57 to about 1.3;
each E₅ and E₈ is each, independently, an amino acid having an isoelectric point of about 2.7 to about 10.8, a molecular weight of about 75 g/mol to about 205 g/mol, a hydropathy index of about −5.1 to about 33.5, and a helicity of about 0.57 to about 1.3;
each E₆ is, independently, an amino acid having an isoelectric point of about 5 to about 10.8, a molecular weight of about 89 g/mol to about 205 g/mol, a hydropathy index of about −5.1 to about 33.5, and a helicity of about 0.57 to about 1.3;
each E₇ is, independently, an amino acid having an isoelectric point of about 5 to about 9.75, a molecular weight of about 75 g/mol to about 205 g/mol, a hydropathy index of about −4 to about 33.5, and a helicity of about 0.79 to about 1.3;
each E₉, E₁₃, and E₁₄ is each, independently, an amino acid having an isoelectric point of about 2.7 to about 10.8, a molecular weight of about 75 g/mol to about 205 g/mol, a hydropathy index of about −5.1 to about 33.5, and a helicity of about 0.57 to about 1.3;
each E₁₀ and E₁₂ is, independently, an amino acid having an isoelectric point of about 5 to about 10.8, a molecular weight of about 75 g/mol to about 205 g/mol, a hydropathy index of about −5.1 to about 34, and a helicity of about 0.5 to about 1.3;
E₁₁ is an amino acid having an isoelectric point of about 5 to about 9.75, a molecular weight of about 89 g/mol to about 205 g/mol, a hydropathy index of about −4 to about 33.5, and a helicity of about 0.79 to about 1.3; and
E₁₅ is an amino acid having an isoelectric point of about 2.7 to about 10.8, a molecular weight of about 75 g/mol to about 205 g/mol, a hydropathy index of about −4 to about 15.5, and a helicity of about 0.57 to about 1.2;
wherein Formula IX is represented as:
F₁-(F₂)ᵥ-(F₃)_{w}-[(F₄)ₓ-(F₅)_{y}]_{z}-F₆-F₇-F₈-[F₉-F₁₀]ₐ (Formula IX)
wherein:
v and w are each, independently, 0, 1, 2, or 3;
x and y are each, independently, 0, 1, 2, 3, or 4;
a is 0 or 1; and
z is 1, 2, 3, 4, 5, 6, 7, or 8;
wherein:
F₁ is an amino acid having an isoelectric point of about 5.4 to about 11, a molecular weight of about 89 g/mol to about 175 g/mol; a hydropathy index of about −4 to about 31, and a helicity or about 0.9 to about 1.3;
each F₂ is, independently, an amino acid having an isoelectric point of about 3 to about 11, a molecular weight of about 89 g/mol to about 205 g/mol; a hydropathy index of about −5.1 to about 34, and a helicity or about 0.5 to about 1.3;
each F₃ and F₇ is each, independently, an amino acid having an isoelectric point of about 3 to about 11, a molecular weight of about 89 g/mol to about 205 g/mol; a hydropathy index of about −5.1 to about 34, and a helicity or about 0.5 to about 1.3;
each F₄ is, independently, an amino acid having an isoelectric point of about 3 to about 11, a molecular weight of about 89 g/mol to about 205 g/mol; a hydropathy index of about −5.1 to about 34, and a helicity or about 0.5 to about 1.3;
each F₅, F₆, F₈, and F₉ is each, independently, an amino acid having an isoelectric point of about 3 to about 11, a molecular weight of about 89 g/mol to about 205 g/mol; a hydropathy index of about −5.1 to about 34, and a helicity or about 0.5 to about 1.3; and
F₁₀ is an amino acid having an isoelectric point of about 3 to about 11, a molecular weight of about 89 g/mol to about 205 g/mol; a hydropathy index of about −5.1 to about 34, and a helicity or about 0.5 to about 1.3; and
wherein Formula XIII is represented as:
L₁-(L₂)ₓ-[(L₃)ₐ-(L₄)ₐ]_{y}-[(L₅)ₐ-(L₆)ₐ-(L₇)ₐ]_{z}-(L₈)ₐ-(L₉)ₐ-(L₁₀)ₐ-(L₁₁)ₐ-(L₁₂)ₐ (Formula XIII)
wherein:
x is 1, 2, or 3;
y is 1, 2, 3, or 4;
z is 5, 6, 7, 8, 9, or 10; and
each a is, independently, 0 or 1;
wherein:
each L₂ is, independently, an amino acid having an isoelectric point of about 2.7 to about 10.8, a molecular weight of about 75 g/mol to about 205 g/mol; a hydropathy index of about −5.1 to about 34, and a helicity or about 0.5 to about 1.3;
each L₃ and L₆ is each, independently, an amino acid having an isoelectric point of about 2.7 to about 10.8, a molecular weight of about 75 g/mol to about 205 g/mol; a hydropathy index of about −5.1 to about 34, and a helicity or about 0.5 to about 1.3;
each L₄, L₇ and L₉ is each, independently, an amino acid having an isoelectric point of about 2.7 to about 10.8, a molecular weight of about 75 g/mol to about 205 g/mol; a hydropathy index of about −5.1 to about 34, and a helicity or about 0.5 to about 1.3;
each L₅, L₈, L₁₀ and L₁₁ is each, independently, an amino acid having an isoelectric point of about 2.7 to about 10.8, a molecular weight of about 75 g/mol to about 205 g/mol; a hydropathy index of about −5.1 to about 34, and a helicity or about 0.5 to about 1.3; and
L₁₂ is an amino acid having an isoelectric point of about 2.7 to about 10.8, a molecular weight of about 75 g/mol to about 205 g/mol; a hydropathy index of about −5.1 to about 34, and a helicity or about 0.5 to about 1.3.

18. The polypeptide of claim 16 or 17 wherein n is 1 and X₁ comprises an amino acid sequence having at least 90%, 91%, 92%, 93%, 94%, 95%, 96%, 97%, 98%, 99%, or 100% identity to an amino acid sequence selected from the group consisting of SEQ ID NO. 1, 2, 3, 4, 5, 6, 7, 8, 9, 10, 11, 12, 13, 14, 15, 16, 28, 31, 32, 33, 55, 70, 71, 72, or 73.

19. The polypeptide of any one of claims 16-18, wherein m is 1 and Y₁ comprises an amino acid sequence selected from the group consisting of Formula VI, Formula VII, Formula VIII, Formula X, Formula XI, Formula XIV, and Formula XV;
wherein Formula VI is represented as:
G₁-G₂-G₃-G₄-G₅-G₆-G₇-G₈-G₉-G₁₀-G₁₁-G₁₂-G₁₃-G₁₄-G₁₅-G₁₆-G₁₇-G₁₈-G₁₉-G₂₀-G₂₁-G₂₂-G₂₃-G₂₄-G₂₅ (Formula VI)
wherein:
G₁ is an amino acid selected from the group consisting of I, L, F, V, A, N, S, D, R, and K;
G₂ is an amino acid selected from the group consisting of P, S, N, G, and E;
G₃ is an amino acid selected from the group consisting of L, F, I, V, Y, A, S, R, and H;
G₄ is an amino acid selected from the group consisting of V, M, P, Y, A, T, S, N, K, and H;
G₅ is an amino acid selected from the group consisting of A, G, R, Y, K, D, M, V, W, I, and L;
G₆ is an amino acid selected from the group consisting of N, R, and K;
G₇ is an amino acid selected from the group consisting of V, P, A, T, Q, G, E, D, R, and K;
G₈ is an amino acid selected from the group consisting of P, Y, T, Q, S, N, W, F, R, K, and H;
G₉ is an amino acid selected from the group consisting of F, L, A, Q, N, S, E, G, D, and H;
G₁₀ is an amino acid selected from the group consisting of H, S, N, D, Q, E, T, Y, M, V, I, and L;
G₁₁ is an amino acid selected from the group consisting of S, R, T, G, K, E, D, and P;
G₁₂ is an amino acid selected from the group consisting of D, E, Q, N, A, and V;
G₁₃ is an amino acid selected from the group consisting of N, S, E, D, T, H, K, A, and P;
G₁₄ is an amino acid selected from the group consisting of G, S, N, H, E, C, Y, L, and F;
G₁₅ is an amino acid selected from the group consisting of S, T, and H;
G₁₆ is an amino acid selected from the group consisting of E, D, Q, N, S, T, K, and A;
G₁₇ is an amino acid selected from the group consisting of W, N, D, and R;
G₁₈ is an amino acid selected from the group consisting of L and F;
G₁₉ is an amino acid selected from the group consisting of Y, V, A, Q, N, S, E, D, L, R, K, and H;
G₂₀ is an amino acid selected from the group consisting of K, R, S, and I;
G₂₁ is R;
G₂₂ is an amino acid selected from the group consisting of D, E, N, S, T, G, A, Y, and L;
G₂₃ and G₂₄ are each, independently, an amino acid selected from the group consisting of V, P, Y, I, A, E, K, F, T, S, G, D, M, and N; and
G₂₅ is an amino acid selected from the group consisting of Y, P, A, T, Q, S, E, F, and H;
wherein Formula VII is represented as:
(H₁)ₘ-(H₂)ₘ-(H₃)ₘ-(H₄)ₘ-(H₅)ₘ-(H₆)ₘ-(H₇)ₘ-(H₈)ₘ-(H₉)ₘ-(H₁₀)ₘ-(H₁₁)ₘ- (H₁₂)ₘ-(H₁₃)ₘ-(H₁₄)ₘ-(H₁₅)ₘ-(H₁₆)ₘ-(H₁₇)ₘ-(H₁₈)ₘ-(H₁₉)ₘ-(H₂₀)ₘ-(H₂₁)ₘ-(H₂₂)ₘ-(H₂₃)ₘ-(H₂₄)ₘ-(H₂₅)ₘ-(H₂₆)ₘ-(H₂₇)ₘ-(H₂₈)ₘ-(H₂₉)ₘ-(H₃₀)ₘ-(H₃₁)ₘ-(H₃₂)ₘ-(H₃₃)ₘ-(H₃₄)ₘ-(H₃₅)ₘ-(H₃₆)ₘ-H₃₇-H₃₈-H₃₉-H₄₀ (Formula VII)
wherein:
each m is, independently, 0, 1, or 2;
wherein:
each H₁ is, independently, an amino acid selected from the group consisting of E, D, S, L, G, Q, and A;
each H₂ and H₂₈ is each, independently, an amino acid selected from the group consisting of P, S, R, T, N, G, D, K, and A;
each H₃ is, independently, an amino acid selected from the group consisting of W and Y;
each H₄ is, independently, an amino acid selected from the group consisting of S, N, A, P, and V;
each H₅ and H₃₀ is each, independently, an amino acid selected from the group consisting of T, Q, A, E, F, and S;
each H₆ is, independently, an amino acid selected from the group consisting of L, F, and I;
each H₇ is, independently, an amino acid selected from the group consisting of F, V, M, T, S, and K;
each H₈ is, independently, an amino acid selected from the group consisting of V, P, I, A, S, and K;
each H₉ and H₁₇ is each, independently, an amino acid selected from the group consisting of T, G, V, W, and A;
each H₁₀ is, independently, an amino acid selected from the group consisting of R, H, S, G, N, E, T, and V;
each H₁₁ is, independently, an amino acid selected from the group consisting of S, G, D, A, and M;
each H₁₂ is, independently, an amino acid selected from the group consisting of T, S, E, G, D, K, and H;
each H₁₃ is, independently, an amino acid selected from the group consisting of L, M, Y, N, S, D, and K;
each H₁₄ is, independently, an amino acid selected from the group consisting of D, Q, N, S, K, and C;
each H₁₅ is, independently, an amino acid selected from the group consisting of E, S, D, L, and G;
each H₁₆ is, independently, an amino acid selected from the group consisting of I, L, V, M, A, and T;
each H₁₈ is, independently, an amino acid selected from the group consisting of D, E, S, T, K, and G;
each H₁₉ is, independently, an amino acid selected from the group consisting of Y, F, and L;
each H₂₀ is, independently, an amino acid selected from the group consisting of N, Q, S, T, R, and F;
each H₂₁ and H₃₄ is each, independently, an amino acid selected from the group consisting of S, K, T, A, Y, M, and F;
each H₂₂ is, independently, an amino acid selected from the group consisting of T, Q, S, D, C, V, and L;
each H₂₃ is, independently, an amino acid selected from the group consisting of G, S, K, N, H, D, W, and L;
each H₂₄ is, independently, an amino acid selected from the group consisting of I, L, V, P, N, and E;
each H₂₅ and H₃₃ is each, independently, an amino acid selected from the group consisting of A, T, G, R, Y, L, F, and E;
each H₂₆ and H₄₀ is each, independently, an amino acid selected from the group consisting of V, I, F, M, L, A, and T;
each H₂₇ is, independently, an amino acid selected from the group consisting of D, E, Q, N, S, A, and I;
each H₂₉ is, independently, an amino acid selected from the group consisting of E, D, T, A, Y, M, V, I, F, and L;
each H₃₁ is, independently, an amino acid selected from the group consisting of F, W, V, M, S, G, and R;
each H₃₂ is, independently, an amino acid selected from the group consisting of H, S, E, G, and T;
each H₃₅ is, independently, an amino acid selected from the group consisting of R, K, S, and Q;
each H₃₆ is, independently, an amino acid selected from the group consisting of H, R, S, T, A, V, W, and L;
H₃₇ is an amino acid selected from the group consisting of K, Q, D, A, and I;
H₃₈ is an amino acid selected from the group consisting of R, K, T, and F; and
H₃₉ is an amino acid selected from the group consisting of D, N, S, T, K, A, Y, and L;
wherein Formula VIII is represented as:
(I₁)ₘ-(I₂)ₘ-(I₃)ₘ-(I₄)ₘ-(I₅)ₘ-(I₆)ₘ-(I₇)ₓ-(I₈)ₘ-(I₉)ₘ-(I₁₀)ₘ-(I₁₁)ₓ- (I₁₂)ₘ-(I₁₃)ₓ-(I₁₄)ₓ-(I₁₅)ₘ-(I₁₆)ₓ-(I₁₇)ₘ-I₁₈-I₁₉-I₂₀-I₂₁-I₂₂-I₂₃ (Formula VIII)
wherein:
each m is, independently, 0, 1, or 2; and
each x is, independently, 0, 1, 2, 3, or 4;
wherein:
each I₁ and I₆ is each, independently, an amino acid selected from the group consisting of S, Q, E, A, I, G, V, R, T, and Y;
each I₂ is, independently, an amino acid selected from the group consisting of T, S, E, R, P, V, I, and F;
each I₃ is, independently, L.
each I₄ is, independently, an amino acid selected from the group consisting of T, N, K, and M;
each I₅ is, independently, an amino acid selected from the group consisting of P, A, and D;
each I₇ is, independently, an amino acid selected from the group consisting of T, S, K, H, Y, V, and F;
each I₈ and I₁₅ is each, independently, an amino acid selected from the group consisting of F, L, W, A, T, M, Y, and C;
each I₉ is, independently, an amino acid selected from the group consisting of I, L, and V;
each I₁₀ and I₁₆ is each, independently, an amino acid selected from the group consisting of G, S, N, E, D, A, K, H, C, P, and F;
each I₁₁ is, independently, an amino acid selected from the group consisting of I, L, V, A, T, and S;
each I₁₂ is, independently, an amino acid selected from the group consisting of T, N, A, E, and G;
each I₁₃ is, independently, an amino acid selected from the group consisting of E, Q, S, T, R, K, A, L, D, and F;
each I₁₄ is, independently, an amino acid selected from the group consisting of T, S, Q, F, A, G, V, I, and L;
each I₁₇ is, independently, an amino acid selected from the group consisting of I, L, V, N, A, T, and S;
I₁₈ and I₂₁ are each, independently, an amino acid selected from the group consisting of R, K, Q, and A;
I₁₉ is an amino acid selected from the group consisting of H, R, S, N, T, A, V, and W;
I₂₀ is an amino acid selected from the group consisting of K, N, Q, D, E, A, and I;
I₂₂ is an amino acid selected from the group consisting of D, N, S, A, Y, and L; and
I₂₃ is an amino acid selected from the group consisting of V, I, L, F, and A;
wherein Formula X is represented as:
(J₁)_{z}-(J₂)_{z}-(J₃)_{z}-(J₄)_{z}-(J₅)_{z}-(J₆)_{z}-(J₇)_{z}-(J₈)_{z}-(J₉)_{z}-(J₁₀)_{z}-(J₁₁)_{z}- (J₁₂)_{z}-(J₁₃)_{z}-(J₁₄)_{z}-(J₁₅)_{z}-(J₁₆)_{z}-(J₁₇)_{z}-(J₁₈)_{z}-(J₁₉)_{z}-(J₂₀)_{z}-(J₂₁)_{z}-J₂₂-J₂₃-J₂₄-J₂₅ (Formula X)
wherein:
each z is, independently, 0, 1, 2, 3, 4, or 5;
wherein:
each J₁ is, independently, an amino acid selected from the group consisting of H, K, G, A, P, F, and L;
each J₂ is, independently, an amino acid selected from the group consisting of D, E, N, G, P, H, T, R, K, and A;
each J₃ is, independently, an amino acid selected from the group consisting of G, A, P, V, and L;
each J₄ is, independently, an amino acid selected from the group consisting of F, I, P, A, S, E, D, R, and K;
each J₅ is, independently, an amino acid selected from the group consisting of S, R, T, G, K, E, D, and C;
each J₆ is, independently, an amino acid selected from the group consisting of T, S, A, D, and F;
each J₇ is, independently, an amino acid selected from the group consisting of D, E, N, G, P, H, T, R, K, and A;
each J₈ is, independently, an amino acid selected from the group consisting of Y, C, A, W, I, S, E, D, F, L, R, and K;
each J₉ is, independently, an amino acid selected from the group consisting of H, K, N, D, G, T, A, C, Y, V, and L;
each J₁₀ is, independently, an amino acid selected from the group consisting of L, V, A, G, E, I, P, and R;
each J₁₁ is, independently, an amino acid selected from the group consisting of I, W, V, Y, P, T, N, S, R, and K;
each J₁₂ is, independently, an amino acid selected from the group consisting of A, G, Q, N, R, Y, E, D, and L;
each J₁₃ is, independently, an amino acid selected from the group consisting of I, L, W, V, M, Y, P, A, S, and G;
each J₁₄ is, independently, an amino acid selected from the group consisting of V, C, L, F, A, T, N, G, and R;
each J₁₅ is, independently, an amino acid selected from the group consisting of G, S, R, K, A, T, H, E, W, L, and F;
each J₁₆ is, independently, an amino acid selected from the group consisting of D, E, Q, S, H, T, R, G, Y, V, F, and L;
each J₁₇ is, independently, an amino acid selected from the group consisting of E, S, G, Y, I, and L;
each J₁₈ is, independently, an amino acid selected from the group consisting of A, S, P, H, and V;
each J₁₉ is, independently, an amino acid selected from the group consisting of N, E, R, K, and A;
each J₂₀ is, independently, an amino acid selected from the group consisting of R, T, V, I, and L;
each J₂₁ is, independently, an amino acid selected from the group consisting of L, V, A, G, E, I, P, and R;
J₂₂ is an amino acid selected from the group consisting of K, R, D, T, M, and W;
J₂₃ is an amino acid selected from the group consisting of R, T, V, I, and L;
J₂₄ is an amino acid selected from the group consisting of S, N, G, E, D, P, and W; and
J₂₅ is an amino acid selected from the group consisting of A, T, S, Y, M, V, and L;
wherein Formula XI is represented as:
(K₁)_{b}-(K₂)_{b}-(K₃)_{b}-(K₄)_{b}-(K₅)_{b}-(K₆)_{b}-(K₇)_{b}-(K₈)_{b}-(K₉)_{b}-(K₁₀)_{b}-(K₁₁)_{b}- (K₁₂)_{b}-(K₁₃)_{b}-(K₁₄)_{b}-(K₁₅)_{b}-(K₁₆)_{b}-(K₁₇)_{b}-(K₁₈)_{b}-(K₁₉)_{b}-(K₂₀)_{b}-(K₂₁)_{b}-(K₂₂)_{b}-(K₂₃)_{b}-(K₂₄)_{b}-(K₂₅)_{b}-(K₂₆)_{b}-(K₂₇)_{b}-(K₂₈)_{b}-(K₂₉)_{b}-(K₃₀)_{b}-(K₃₁)_{b}-(K₃₂)_{b}-(K₃₃)_{b}-(K₃₄)_{b}-(K₃₅)_{b}-(K₃₆)_{b}-(K₃₇)_{b}-(K₃₈)_{b}-(K₃₉)_{b}-(K₄₀)_{b}-(K₄₁)_{b}-(K₄₂)_{b}-(K₄₃)_{b}-(K₄₄)_{b}-(K₄₅)_{b}-(K₄₆)_{b}-(K₄₇)_{b}-(K₄₈)_{b}-(K₄₉)_{b}-(K₅₀)_{b}-(K₅₁)_{b}-(K₅₂)_{b}-(K₅₃)_{b}-(K₅₄)_{b}-(K₅₅)_{b}-(K₅₆)_{b}-(K₅₇)_{b}-(K₅₈)_{b}-(K₅₉)_{b}-(K₆₀)_{b}-(K₆₁)_{b}-(K₆₂)_{b}-(K₆₃)_{b}-(K₆₄)_{b}-(K₆₅)_{b}-(K₆₆)_{b}-(K₆₇)_{b}-(K₆₈)_{b}-(K₆₉)_{b}-(K₇₀)_{b}-(K₇₁)_{b}-(K₇₂)_{b}-(K₇₃)_{b}-(K₇₄)_{b}-(K₇₅)_{b}-(K₇₆)_{b}-(K₇₇)_{b}-(K₇₈)_{b}-(K₇₉)_{b}-(K₈₀)_{b}-(K₈₁)_{b}-(K₈₂)_{b}-(K₈₃)_{b}-(K₈₄)_{b}-(K₈₅)_{b}-(K₈₆)_{b}-(K₈₇)_{b}-(K₈₈)_{b}-K₈₉-K₈₉-K₈₉-K₈₉-K₈₉ (Formula XI)
wherein:
each b is, independently, 0, 1, 2, or 3;
wherein:
each K₁ is, independently, an amino acid selected from the group consisting of S, G, D, A, C, P, and Y;
each K₂ is, independently, an amino acid selected from the group consisting of Q, S, E, T, R, K, G, A, Y, M, V, and I;
each K₃ is, independently, an amino acid selected from the group consisting of G, S, N, T, Q, D, P, L, F, V, K, A, and C;
each K₄ is, independently, an amino acid selected from the group consisting of R, G, N, D, A, P, Y, and L;
each K₅ is, independently, an amino acid selected from the group consisting of E, A, V, Q, G, Y, M, I, and L;
each K₆ is, independently, an amino acid selected from the group consisting of S, Q, R, T, D, G, E, A, and K;
each K₇ is, independently, an amino acid selected from the group consisting of N, Q, R, H, K, A, I, F, and L;
each K₈ is, independently, an amino acid selected from the group consisting of A, T, Q, G, R, K, D, L, F, C, V, S, and H;
each K₉ is, independently, an amino acid selected from the group consisting of G, S, N, T, Q, D, P, L, F, V, K, A, and C;
each K₁₀ is, independently, an amino acid selected from the group consisting of K, H, E, A, Y, L, and F;
each K₁₁ is, independently, an amino acid selected from the group consisting of S, T, K, E, A, C, W, F, and L;
each K₁₂ is, independently, an amino acid selected from the group consisting of K, R, H, S, Q, D, E, and A;
each K₁₃ is, independently, an amino acid selected from the group consisting of G, S, T, E, P, W, R, N, and Q;
each K₁₄ is, independently, an amino acid selected from the group consisting of D, Q, S, G, V, E, N, H, R, P, and F;
each K₁₅ is, independently, an amino acid selected from the group consisting of C, A, M, V, S, E, G, I, F, and L;
each K₁₆ is, independently, an amino acid selected from the group consisting of R, K, S, Q, T, Y, N, V, I, L, and C;
each K₁₇ is, independently, an amino acid selected from the group consisting of A, G, S, Q, Y, E, D, H, and I;
each K₁₈ is, independently, an amino acid selected from the group consisting of R, K, S, Q, T, Y, N, V, I, L, and C;
each K₁₉ is, independently, an amino acid selected from the group consisting of E, D, T, H, K, G, P, V, and L;
each K₂₀ is, independently, an amino acid selected from the group consisting of F, L, I, V, M, T, G, and R;
each K₂₁ is, independently, an amino acid selected from the group consisting of E, D, S, G, A, C, and P;
each K₂₂ is, independently, an amino acid selected from the group consisting of D, T, G, A, Y, N, S, C, P, W, and I;
each K₂₃ is, independently, an amino acid selected from the group consisting of G, S, N, E, D, Y, and L;
each K₂₄ is, independently, an amino acid selected from the group consisting of T, S, E, G, P, and I;
each K₂₅ is, independently, an amino acid selected from the group consisting of K, S, G, T, and L;
each K₂₆ is, independently, an amino acid selected from the group consisting of S, G, K, E, D, P, and F;
each K₂₇ is, independently, an amino acid selected from the group consisting of P, A, E, L, T, Q, S, G, K, Y, F, C, V, W, and R;
each K₂₈ is, independently, an amino acid selected from the group consisting of E, D, Q, S, T, P, and L;
each K₂₉ is, independently, an amino acid selected from the group consisting of A, T, S, E, V, W, and I;
each K₃₀ is, independently, an amino acid selected from the group consisting of K, H, S, G, N, Q, P, and Y;
each K₃₁ is, independently, an amino acid selected from the group consisting of L, F, V, P, A, N, G, and H;
each K₃₂ is, independently, an amino acid selected from the group consisting of A, G, N, P, R, E, and K;
each K₃₃ is, independently, an amino acid selected from the group consisting of R, S, N, A, P, Y, V, I, F, and G;
each K₃₄ is, independently, an amino acid selected from the group consisting of E, S, T, V, I, H, A, P, F, and L;
each K₃₅ is, independently, an amino acid selected from the group consisting of A, T, Q, P, R, V, N, E, and L;
each K₃₆ is, independently, an amino acid selected from the group consisting of R, K, H, G, Q, D, T, Y, and F;
each K₃₇ is, independently, an amino acid selected from the group consisting of D, E, N, T, C, Y, V, I, and L;
each K₃₈ is, independently, an amino acid selected from the group consisting of S, Q, R, T, D, G, E, A, and K;
each K₃₉ is, independently, an amino acid selected from the group consisting of K, S, G, Q, D, E, A, M, I, and L;
each K₄₀ is, independently, an amino acid selected from the group consisting of H, K, S, D, E, T, P, and L;
each K₄₁ is, independently, an amino acid selected from the group consisting of A, T, S, N, P, V, L, and F;
each K₄₂ is, independently, an amino acid selected from the group consisting of K, D, M, V, I, L, and F;
each K₄₃ is, independently, an amino acid selected from the group consisting of G, S, N, T, Q, D, P, L, F, V, K, A, and C;
each K₄₄ is, independently, an amino acid selected from the group consisting of L, T, F, V, P, A, K, and I;
each K₄₅ is, independently, an amino acid selected from the group consisting of G, S, K, N, T, Q, D, A, P, L, F, and V;
each K₄₆ is, independently, an amino acid selected from the group consisting of L, F, Q, S, G, and D;
each K₄₇ is, independently, an amino acid selected from the group consisting of S, R, E, A, P, V, W, and L;
each K₄₈ is, independently, an amino acid selected from the group consisting of A, S, V, G, Q, R, E, D, L, T, K, F, C, and H;
each K₄₉ is, independently, an amino acid selected from the group consisting of E, S, T, R, G, A, P, and L;
each K₅₀ is, independently, an amino acid selected from the group consisting of S, N, R, A, P, and Y;
each K₅₁ is, independently, an amino acid selected from the group consisting of G, A, T, H, M, V, L, and F;
each K₅₂ is, independently, an amino acid selected from the group consisting of S, T, H, A, C, M, and L;
each K₅₃ is, independently, an amino acid selected from the group consisting of G, S, T, E, P, W, R, N, and Q;
each K₅₄ is, independently, an amino acid selected from the group consisting of S, H, Y, F, N, Q, R, T, G, and K;
each K₅₅ is, independently, an amino acid selected from the group consisting of A, T, Q, E, M, V, I, L, and F;
each K₅₆ is, independently, an amino acid selected from the group consisting of S, N, E, A, P, F, and L;
each K₅₇ is, independently, an amino acid selected from the group consisting of D, S, R, K, A, V, W, I, and F;
each K₅₈ is, independently, an amino acid selected from the group consisting of K, S, G, D, T, L, R, E, Y, and N;
each K₅₉ is, independently, an amino acid selected from the group consisting of S, R, G, A, V, and F;
each K₆₀ is, independently, an amino acid selected from the group consisting of A, T, Q, G, R, K, D, L, F, C, V, S, and H;
each K₆₁ is, independently, an amino acid selected from the group consisting of R, S, G, N, E, T, A, and V;
each K₆₂ is, independently, an amino acid selected from the group consisting of E, S, T, V, I, H, A, P, F, and L;
each K₆₃ is, independently, an amino acid selected from the group consisting of A, G, S, Q, R, E, D, V, L, T, K, F, C, and H;
each K₆₄ is, independently, an amino acid selected from the group consisting of E, A, V, Q, G, Y, M, I, and L;
each K₆₅ is, independently, an amino acid selected from the group consisting of G, S, T, E, P, W, R, N, and Q;
each K₆₆ is, independently, an amino acid selected from the group consisting of A, G, P, M, N, V, and S;
each K₆₇ is, independently, an amino acid selected from the group consisting of T, Q, E, N, S, A, Y, V, W, and F;
each K₆₈ is, independently, an amino acid selected from the group consisting of I, V, P, and A;
each K₆₉ is, independently, an amino acid selected from the group consisting of D, Q, S, G, V, E, N, H, R, P, and F;
each K₇₀ is, independently, an amino acid selected from the group consisting of G, S, R, N, T, Y, L, and F;
each K₇₁ is, independently, an amino acid selected from the group consisting of E, D, N, S, T, H, and Y;
each K₇₂ is, independently, an amino acid selected from the group consisting of L, I, W, V, A, T, S, E, R, and K;
each K₇₃ is, independently, an amino acid selected from the group consisting of G, S, K, A, C, F, N, T, Q, D, P, L, and V;
each K₇₄ is, independently, an amino acid selected from the group consisting of A, S, N, P, K, V, I, and L;
each K₇₅ is, independently, an amino acid selected from the group consisting of P, A, E, L, T, Q, S, G, K, Y, F, C, V, W, and R;
each K₇₆ is, independently, an amino acid selected from the group consisting of L, T, F, V, P, A, K, and I;
each K₇₇ is, independently, an amino acid selected from the group consisting of M, V, Y, L, A, N, E, and H;
each K₇₈ is, independently, an amino acid selected from the group consisting of D, T, G, A, Y, N, S, C, P, W, and I;
each K₇₉ is, independently, an amino acid selected from the group consisting of A, S, V, G, Q, R, E, D, L, T, K, F, C, and H;
each K₈₀ is, independently, an amino acid selected from the group consisting of K, R, S, A, P, V, I, and L;
each K₈₁ is, independently, an amino acid selected from the group consisting of F, L, V, A, T, S, E, D, R, and K;
each K₈₂ is, independently, an amino acid selected from the group consisting of L, F, M, A, N, G, and E;
each K₈₃ is, independently, an amino acid selected from the group consisting of D, S, H, A, V, I, F, and L;
each K₈₄ is, independently, an amino acid selected from the group consisting of A, T, Q, S, R, V, L, G, H, F, K, D, and C;
each K₈₅ is, independently, an amino acid selected from the group consisting of T, Q, E, N, S, A, Y, V, W, and F;
each K₈₆ is, independently, an amino acid selected from the group consisting of A, P, R, Y, K, D, M, L, and F;
each K₈₇ is, independently, an amino acid selected from the group consisting of N, S, D, T, A, P, and L;
each K₈₈ is, independently, an amino acid selected from the group consisting of R, S, N, A, P, Y, V, I, F, and G;
K₈₉ is an amino acid selected from the group consisting of K, R, H, G, E, T, Y, and I;
K₉₀ is an amino acid selected from the group consisting of R, S, G, N, Q, A, Y, and W;
K₉₁ is an amino acid selected from the group consisting of V, I, and F;
K₉₂ is an amino acid selected from the group consisting of A, G, P, M, N, V, and S; and
K₉₃ is an amino acid selected from the group consisting of E, D, Q, S, R, K, M, and L
wherein Formula XIV is represented as:
(M₁)_{b}-(M₂)_{b}-(M₃)_{b}-(M₄)_{b}-(M₅)_{b}-(M₆)_{b}-(M₇)_{b}-(M₈)_{b}-(M₉)_{b}-(M₁₀)_{b}-(M₁₁)_{b}- (M₁₂)_{b}-(M₁₃)_{b}-(M₁₄)_{b}-(M₁₅)_{b}-(M₁₆)_{b}-(M₁₇)_{b}-(M₁₈)_{b}-(M₁₉)_{b}-(M₂₀)_{b}-(M₂₁)_{b}-(M₂₂)_{b}-(M₂₃)_{b}-(M₂₄)_{b}-(M₂₅)_{b}-(M₂₆)_{b}-(M₂₇)_{b}-(M₂₈)_{b}-(M₂₉)_{b}-(M₃₀)_{b}-(M₃₁)_{b}-(M₃₂)_{b}-(M₃₃)_{b}-(M₃₄)_{b}-(M₃₅)_{b}-(M₃₆)_{b}-(M₃₇)_{b}-(M₃₈)_{b}-(M₃₉)_{b}-(M₄₀)_{b}-(M₄₁)_{b}-(M₄₂)_{b}-(M₄₃)_{b}-(M₄₄)_{b}-(M₄₅)_{b}-(M₄₆)_{b}-(M₄₇)_{b}-(M₄₈)_{b}-(M₄₉)_{b}-(M₅₀)_{b}-(M₅₁)_{b}-(M₅₂)_{b}-(M₅₃)_{b}-(M₅₄)_{b}-(M₅₅)_{b}-(M₅₆)_{b}-(M₅₇)_{b}-(M₅₈)_{b}-(M₅₉)_{b}-(M₆₀)_{b}-(M₆₁)_{b}-(M₆₂)_{b}-(M₆₃)_{b}-(M₆₄)_{b}-(M₆₅)_{b}-(M₆₆)_{b}-(M₆₇)_{c}-(M₆₈)_{c}-(M₆₉)_{c}-(M₇₀), (Formula XIV);
wherein:
each b is, independently, 0, 1, 2, or 3; and
each c is, independently, 1 or 2;
wherein:
each M₁ is, independently, an amino acid selected from the group consisting of A, T, C, S, Y, E, H, V, W, I, L, F, G, Q, N, P, R, K, D, and M;
each M₂ is, independently, an amino acid selected from the group consisting of S, T, A, N, R, G, E, P, V, F, L, Q, K, H, D, I, C, Y, M, and W;
each M₃ is, independently, an amino acid selected from the group consisting of G, S, R, A, T, Q, E, D, C, Y, V, I, L, and N;
each M₄ is, independently, an amino acid selected from the group consisting of R, H, N, Q, E, A, Y, M, V, W, F, and L;
each M₅ is, independently, an amino acid selected from the group consisting of P, Y, A, T, Q, S, G, D, R, K, C, V, I, L, and H;
each M₆ is, independently, an amino acid selected from the group consisting of T, Q, N, S, A, E, G, D, H, P, F, L, C, K, V, R, Y, I, M, and W;
each M₇ is, independently, an amino acid selected from the group consisting of A, G, S, Q, N, K, D, T, C, Y, E, H, V, W, I, L, F, P, R, and M;
each M₈ is, independently, an amino acid selected from the group consisting of T, Q, N, S, A, G, C, R, K, P, Y, M, V, I, L, F, E, W, D, and H;
each M₉ is, independently, an amino acid selected from the group consisting of G, S, H, P, R, A, T, Q, E, D, C, Y, V, I, L, N, W, F, K, and M;
each M₁₀ is, independently, an amino acid selected from the group consisting of Q, E, and W;
each M₁₁ is, independently, an amino acid selected from the group consisting of V, I, L, F, C, A, and T;
each M₁₂ is, independently, an amino acid selected from the group consisting of S, G, A, N, Q, R, T, K, E, H, D, P, I, F, V, C, Y, L, M, and W;
each M₁₃ is, independently, an amino acid selected from the group consisting of T, Q, N, S, D, P, F, A, E, G, H, L, C, K, V, R, Y, I, M, and W;
each M₁₄ is, independently, an amino acid selected from the group consisting of L, F, I, V, M, Y, A, T, Q, N, S, D, K, P, E, R, H, G, and C;
each M₁₅ is, independently, an amino acid selected from the group consisting of S, P, V, E, T, A, F, L, N, R, G, Q, K, H, D, I, C, Y, M, and W;
each M₁₆ is, independently, an amino acid selected from the group consisting of T, S, A, E, G, C, R, P, Y, M, V, W, I, F, L, Q, N, D, H, and K;
each M₁₇ is, independently, an amino acid selected from the group consisting of D, E, Q, T, K, P, F, N, S, G, A, Y, R, and V;
each M₁₈ is, independently, an amino acid selected from the group consisting of G, S, H, P, R, D, N, A, T, Q, E, C, Y, V, I, L, W, F, K, and M;
each M₁₉ is, independently, an amino acid selected from the group consisting of T, P, F, S, A, E, G, C, R, Y, M, V, W, I, L, Q, N, D, H, and K;
each M₂₀ is, independently, an amino acid selected from the group consisting of L, F, I, V, Y, A, T, Q, S, D, M, N, K, P, E, R, H, G, and C;
each M₂₁ is, independently, an amino acid selected from the group consisting of F, L, W, Y, and P;
each M₂₂ is, independently, an amino acid selected from the group consisting of P, K, Y, A, T, Q, S, G, D, R, C, V, I, L, and H;
each M₂₃ is, independently, an amino acid selected from the group consisting of T, P, F, S, A, E, G, C, R, Y, M, V, W, I, L, Q, N, D, H, and K
each M₂₄ is, independently, an amino acid selected from the group consisting of S, T, A, N, R, G, E, P, V, F, L, Q, K, H, D, I, C, Y, M, and W;
each M₂₅ is, independently, an amino acid selected from the group consisting of F, W, Y, and P;
each M₂₆ is, independently, an amino acid selected from the group consisting of T, P, F, Q, N, S, A, E, G, D, K, Y, C, V, I, L, and H;
each M₂₇ is, independently, an amino acid selected from the group consisting of D, E, Q, N, S, T, R, K, G, A, Y, P, V, and F;
each M₂₈ is, independently, an amino acid selected from the group consisting of T, Q, N, S, A, G, C, R, K, P, Y, M, V, I, L, F, E, W, D, and H;
each M₂₉ is, independently, an amino acid selected from the group consisting of S, T, E, A, P, V, F, L, N, R, G, Q, K, H, D, I, C, Y, M, and W;
each M₃₀ is, independently, an amino acid selected from the group consisting of D, Q, N, H, K, G, C, and Y;
each M₃₁ is, independently, an amino acid selected from the group consisting of F, L, W, Y, and P;
each M₃₂ is, independently, an amino acid selected from the group consisting of S, T, E, A, P, V, F, L, N, R, G, Q, K, H, D, I, C, Y, M, and W;
each M₃₃ is, independently, an amino acid selected from the group consisting of A, G, S, Q, N, K, D, T, C, Y, E, H, V, W, I, L, F, P, R, and M;
each M₃₄ is, independently, an amino acid selected from the group consisting of T, A, V, I, P, F, Q, N, S, E, G, D, K, Y, C, L, and H;
each M₃₅ is, independently, an amino acid selected from the group consisting of G, S, R, N, H, D, P, A, T, Q, E, C, Y, V, I, L, W, F, K, and M;
each M₃₆ is, independently, an amino acid selected from the group consisting of T, Q, S, A, E, D, K, H, P, Y, V, W, I, F, L, N, G, and C;
each M₃₇ is, independently, an amino acid selected from the group consisting of I, L, W, V, and M;
each M₃₈ is, independently, an amino acid selected from the group consisting of A, G, S, Q, N, K, D, C, P, R, Y, E, V, W, T, H, M, and F;
each M₃₉ is, independently, an amino acid selected from the group consisting of S, T, E, P, V, A, F, L, N, R, G, Q, K, H, D, I, C, Y, M, and W;
each M₄₀ is, independently, an amino acid selected from the group consisting of T, S, A, D, P, M, Q, E, K, H, Y, V, W, I, F, L, N, G, and C;
each M₄₁ is, independently, an amino acid selected from the group consisting of L, F, I, V, Y, A, T, Q, S, D, M, N, K, P, E, R, H, G, and C;
each M₄₂ is, independently, an amino acid selected from the group consisting of P, Y, A, T, Q, S, N, W, G, I, E, D, L, K, and H;
each M₄₃ is, independently, an amino acid selected from the group consisting of S, E, P, V, T, A, F, L, N, R, G, Q, K, H, D, I, C, Y, M, and W;
each M₄₄ is, independently, an amino acid selected from the group consisting of N, Q, S, E, D, T, H, K, G, A, P, W, and F;
each M₄₅ is, independently, an amino acid selected from the group consisting of V, I, L, F, C, A, and T;
each M₄₆ is, independently, an amino acid selected from the group consisting of A, T, S, N, R, Y, K, D, H, M, L, F, G, Q, C, P, E, V, and W;
each M₄₇ is, independently, an amino acid selected from the group consisting of I, L, and V;
each M₄₈ is, independently, an amino acid selected from the group consisting of S, P, V, E, T, A, F, L, N, R, G, Q, K, H, D, I, C, Y, M, and W;
each M₄₉ is, independently, an amino acid selected from the group consisting of F, V, A, T, Q, N, S, E, G, D, and H;
each M₅₀ is, independently, an amino acid selected from the group consisting of L, F, I, V, Y, A, T, Q, S, D, M, N, K, P, E, R, H, G, and C;
each M₅₁ is, independently, an amino acid selected from the group consisting of G, S, R, H, D, P, N, A, T, Q, E, C, Y, V, I, L, W, F, K, and M;
each M₅₂ is, independently, an amino acid selected from the group consisting of T, N, S, G, C, R, H, A, D, P, M, Q, E, K, Y, V, W, I, F, and L;
each M₅₃ is, independently, an amino acid selected from the group consisting of I, L, W, V, and M;
each M₅₄ is, independently, an amino acid selected from the group consisting of P, K, Y, A, T, Q, S, G, D, R, C, V, I, L, and H;
each M₅₅ is, independently, an amino acid selected from the group consisting of D, E, Q, N, S, K, G, A, Y, P, F, T, R, and V;
each M₅₆ is, independently, an amino acid selected from the group consisting of L, F, I, V, Y, P, A, T, Q, N, S, G, E, D, K, H, M, C, and R;
each M₅₇ is, independently, an amino acid selected from the group consisting of S, P, V, E, T, A, F, L, N, R, G, Q, K, H, D, I, C, Y, M, and W;
each M₅₈ is, independently, an amino acid selected from the group consisting of P, M, V, I, L, and F;
each M₅₉ is, independently, an amino acid selected from the group consisting of N, Q, S, E, D, T, R, K, G, A, and Y;
each M₆₀ is, independently, an amino acid selected from the group consisting of G, S, H, P, R, D, N, A, T, Q, E, C, Y, V, I, L, W, F, K, and M;
each M₆₁ is, independently, an amino acid selected from the group consisting of S, P, V, T, A, R, K, E, H, C, Y, I, F, L, N, Q, G, D, M, and W;
each M₆₂ is, independently, an amino acid selected from the group consisting of P, K, A, Y, T, Q, S, G, D, R, C, V, I, L, and H;
each M₆₃ is, independently, an amino acid selected from the group consisting of A, G, S, N, E, K, D, H, M, V, W, I, L, F, T, R, Y, Q, C, and P;
each M₆₄ is, independently, an amino acid selected from the group consisting of D, E, Q, T, K, P, F, N, S, G, A, Y, R, and V;
each M₆₅ is, independently, an amino acid selected from the group consisting of L, V, F, I, Y, P, A, T, Q, N, S, G, E, D, K, H, M, C, and R;
each M₆₆ is, independently, an amino acid selected from the group consisting of S, N, R, T, G, K, E, H, D, A, P, V, C, Y, I, F, L, Q, M, and W;
each M₆₇ is, independently, an amino acid selected from the group consisting of K, R, H, S, G, N, Q, D, E, T, A, C, P, Y, M, V, W, I, L, and F;
each M₆₈ is, independently, an amino acid selected from the group consisting of R, K, H, S, G, N, Q, D, E, T, A, C, P, Y, M, V, W, I, L, and F;
each M₆₉ is, independently, an amino acid selected from the group consisting of S, A, N, Q, R, T, G, K, E, H, D, A, C, P, Y, M, V, W, I, F, and L; and
each M₇₀ is, independently, an amino acid selected from the group consisting of T, Q, N, S, A, E, G, D, C, R, K, H, P, Y, M, V, W, I, F, and L; and
wherein Formula XV is represented as:
(N₁)_{b}-(N₂)_{b}-(N₃)_{b}-(N₄)_{b}-(N₅)_{b}-(N₆)_{b}-(N₇)_{b}-(N₈)_{b}-(N₉)_{b}-(N₁₀)_{b}-(N₁₁)_{b}- (N₁₂)_{b}-(N₁₃)_{b}-(N₁₄)_{b}-(N₁₅)_{b}-(N₁₆)_{b}-(N₁₇)_{b}-(N₁₈)_{b}-(N₁₉)_{b}-(N₂₀)_{b}-(N₂₁)_{b}-(N₂₂)_{b}-(N₂₃)_{b}-(N₂₄)_{b}-(N₂₅)_{b}-(N₂₆)_{b}-(N₂₇)_{b}-(N₂₈)_{b}-(N₂₉)_{b}-(N₃₀)_{b}-(N₃₁)_{b}-(N₃₂)_{b}-(N₃₃)_{b}-(N₃₄)_{b}-(N₃₅)_{b}-(N₃₆)_{b}-(N₃₇)_{b}-(N₃₈)_{b}-(N₃₉)_{b}-(N₄₀)_{b}-(N₄₁)_{b}-(N₄₂)_{b}-(N₄₃)_{b}-(N₄₄)_{b}-(N₄₅)_{b}-(N₄₆)_{b}-(N₄₇)_{b}-(N₄₈)_{b}-(N₄₉)_{b}-(N₅₀)_{b}-(N₅₁)_{b}-(N₅₂)_{b}-(N₅₃)_{b}-(N₅₄)_{b}-(N₅₅)_{b}-(N₅₆)_{b}-(N₅₇)_{b}-(N₅₈)_{b}-(N₅₉)_{b}-(N₆₀)_{b}-(N₆₁)_{b}-(N₆₂)_{b}-(N₆₃)_{b}-(N₆₄)_{b}-(N₆₅)_{b}-(N₆₆)_{b}-(N₆₇)_{c}-(N₆₈)_{c}-(N₆₉)ᵣ-(N₇₀)ᵣ-(N₇₁)_{c} (Formula XV);
wherein:
each b is, independently, 0, 1, 2, or 3; and
each c is, independently, 1 or 2;
wherein:
each N₁ is, independently, an amino acid selected from the group consisting of S, N, D, Q, R, T, G, E, H, A, P, M, V, K, Y, W, F, L, I, and C;
each N₂ is, independently, an amino acid selected from the group consisting of P, A, S, Y, V, T, G, I, E, and C;
each N₃ is, independently, an amino acid selected from the group consisting of T, S, G, D, C, A, L, N, R, P, Y, V, W, I, and F;
each N₄ is, independently, an amino acid selected from the group consisting of S, R, E, A, Q, K, N, D, T, G, H, C, P, Y, I, F, L, M, V, and W;
each N₅ is, independently, an amino acid selected from the group consisting of T, Q, N, G, C, M, S, A, E, D, Y, V, I, F, L, and W;
each N₆ is, independently, an amino acid selected from the group consisting of I, V, L, F, W, Y, A, T, S, E, D, and H;
each N₇ is, independently, an amino acid selected from the group consisting of P, V, A, S, N, G, E, L, and K;
each N₈ is, independently, an amino acid selected from the group consisting of A, G, Q, T, S, N, P, R, D, V, K, C, Y, W, I, L, and F;
each N₉ is, independently, an amino acid selected from the group consisting of F, Y, A, T, N, and R;
each N₁₀ is, independently, an amino acid selected from the group consisting of T, Q, N, R, K, M, S, E, D, H, P, V, W, I, F, and L;
each N₁₁ is, independently, an amino acid selected from the group consisting of A, G, Q, T, S, N, P, R, D, V, K, C, Y, W, I, L, and F;
each N₁₂ is, independently, an amino acid selected from the group consisting of S, N, Q, R, T, G, K, E, H, D, A, P, L, M, V, Y, W, F, I, and C;
each N₁₃ is, independently, an amino acid selected from the group consisting of L, F, I, W, V, M, Y, C, A, T, Q, N, S, G, E, D, and R;
each N₁₄ is, independently, an amino acid selected from the group consisting of V, I, L, A, T, S, G, R, P, Y, N, H, C, M, F, Q, E, K, and D;
each N₁₅ is, independently, an amino acid selected from the group consisting of S, N, Q, T, G, K, E, H, D, A, C, P, Y, I, F, L, R, M, V, and W;
each N₁₆ is, independently, an amino acid selected from the group consisting of T, N, S, A, D, R, P, Y, V, W, I, F, and L;
each N₁₇ is, independently, an amino acid selected from the group consisting of S, N, Q, R, K, E, D, A, T, G, H, C, P, Y, I, F, L, M, V, and W;
each N₁₈ is, independently, an amino acid selected from the group consisting of V, A, T, S, G, R, W, I, C, L, F, E, D, K, P, Y, N, H, M, and Q;
each N₁₉ is, independently, an amino acid selected from the group consisting of T, Q, N, S, A, E, G, D, Y, M, V, I, F, L, and W;
each N₂₀ is, independently, an amino acid selected from the group consisting of S, Q, R, K, E, A, N, D, T, G, H, C, P, Y, I, F, L, M, V, and W;
each N₂₁ is, independently, an amino acid selected from the group consisting of V, W, I, C, L, F, A, T, S, E, D, K, G, R, P, Y, N, H, M, and Q;
each N₂₂ is, independently, an amino acid selected from the group consisting of T, Q, N, S, A, D, C, K, P, Y, M, V, W, I, F, G, E, H, R, and L;
each N₂₃ is, independently, an amino acid selected from the group consisting of L, F, I, V, P, A, T, Q, S, G, R, K, H, M, Y, and D;
each N₂₄ is, independently, an amino acid selected from the group consisting of T, Q, S, A, G, P, Y, I, K, H, V, F, L, N, D, C, M, W, E, and R;
each N₂₅ is, independently, an amino acid selected from the group consisting of S, R, E, A, Q, K, N, D, T, G, H, C, P, Y, I, F, L, M, V, and W;
each N₂₆ is, independently, an amino acid selected from the group consisting of T, N, D, S, A, R, P, Y, V, W, I, F, and L;
each N₂₇ is, independently, an amino acid selected from the group consisting of D, N, R, E, Q, S, H, T, K, G, W, I, P, and Y;
each N₂₈ is, independently, an amino acid selected from the group consisting of V, A, T, S, G, R, W, I, C, L, F, E, D, K, P, Y, N, H, M, and Q;
each N₂₉ is, independently, an amino acid selected from the group consisting of T, S, A, D, C, L, N, R, P, Y, V, W, I, and F;
each N₃₀ is, independently, an amino acid selected from the group consisting of P, Y, V, A, T, S, G, I, E, and C;
each N₃₁ is, independently, an amino acid selected from the group consisting of T, Q, S, A, G, K, H, P, Y, V, I, F, L, N, D, C, M, W, E, and R;
each N₃₂ is, independently, an amino acid selected from the group consisting of S, R, E, A, Q, K, N, D, T, G, H, C, P, Y, I, F, L, M, V, and W;
each N₃₃ is, independently, an amino acid selected from the group consisting of E, D, Q, N, S, T, H, R, G, A, P, F, and L;
each N₃₄ is, independently, an amino acid selected from the group consisting of D, N, R, E, Q, S, H, T, K, G, W, I, P, and Y;
each N₃₅ is, independently, an amino acid selected from the group consisting of T, Q, S, A, G, P, Y, I, K, H, V, F, L, N, D, C, M, W, E, and R;
each N₃₆ is, independently, an amino acid selected from the group consisting of G, S, K, A, T, Q, D, C, P, Y, V, W, I, L, and F;
each N₃₇ is, independently, an amino acid selected from the group consisting of F, Y, A, T, N, and R;
each N₃₈ is, independently, an amino acid selected from the group consisting of V, A, T, S, G, R, W, I, C, L, F, E, D, K, P, Y, N, H, M and Q;
each N₃₉ is, independently, an amino acid selected from the group consisting of L, F, I, W, V, M, C, A, T, Q, N, S, G, D, R, K, and H;
each N₄₀ is, independently, an amino acid selected from the group consisting of P, A, S, Y, V, T, G, I, E, and C;
each N₄₁ is, independently, an amino acid selected from the group consisting of D, N, R, G, Y, E, Q, S, H, T, K, W, and I;
each N₄₂ is, independently, an amino acid selected from the group consisting of S, R, E, A, N, T, G, P, V, Q, K, H, D, Y, M, I, F, L, C, and W;
each N₄₃ is, independently, an amino acid selected from the group consisting of G, S, R, K, A, N, Q, H, E, D, P, W, L, and F;
each N₄₄ is, independently, an amino acid selected from the group consisting of T, Q, S, A, G, P, Y, I, N, E, D, C, K, H, R, V, L, M, F, and W;
each N₄₅ is, independently, an amino acid selected from the group consisting of S, T, G, A, V, I, R, E, N, P, Q, K, H, D, Y, M, F, L, C, and W;
each N₄₆ is, independently, C;
each N₄₇ is, independently, an amino acid selected from the group consisting of S, N, R, T, G, K, E, H, D, A, P, Y, V, W, I, L, Q, M, F, and C;
each N₄₈ is, independently, an amino acid selected from the group consisting of G, S, R, K, N, T, Q, H, E, D, P, I, and L;
each N₄₉ is, independently, an amino acid selected from the group consisting of T, S, G, D, C, A, L, N, R, P, Y, V, W, I, and F;
each N₅₀ is, independently, an amino acid selected from the group consisting of V, A, T, S, G, I, R, P, Y, L, N, H, C, M, F, Q, E, and K;
each N₅₁ is, independently, an amino acid selected from the group consisting of A, T, G, S, Q, N, R, Y, E, H, M, V, W, I, L, and F;
each N₅₂ is, independently, an amino acid selected from the group consisting of D, E, Q, N, S, T, K, A, Y, P, M, W, I, F, and L;
each N₅₃ is, independently, an amino acid selected from the group consisting of A, T, C, G, S, N, P, R, K, D, H, M, and F;
each N₅₄ is, independently, an amino acid selected from the group consisting of L, F, I, V, P, A, T, Q, S, G, R, K, H, M, Y, and D;
each N₅₅ is, independently, an amino acid selected from the group consisting of E, D, N, T, R, K, G, A, and V;
each N₅₆ is, independently, an amino acid selected from the group consisting of A, G, Q, T, S, N, P, R, D, V, W, K, C, Y, I, L, and F;
each N₅₇ is, independently, an amino acid selected from the group consisting of Y, C, N, I, F, and L;
each N₅₈ is, independently, an amino acid selected from the group consisting of S, T, G, H, A, P, Y, V, F, L, N, R, K, E, D, W, I, Q, M, and C;
each N₅₉ is, independently, an amino acid selected from the group consisting of I, V, and L;
each N₆₀ is, independently, S
each N₆₁ is, independently, an amino acid selected from the group consisting of G, S, R, K, A, N, T, Q, E, D, P, and Y;
each N₆₂ is, independently, an amino acid selected from the group consisting of I, V, L, F, W, Y, A, T, S, E, D, and H;
each N₆₃ is, independently, an amino acid selected from the group consisting of T, Q, N, G, C, M, S, A, E, D, Y, V, I, F, L, and W
each N₆₄ is, independently, an amino acid selected from the group consisting of S, N, Q, R, G, K, E, D, P, Y, W, F, T, H, A, V, L, I, M, and C;
each N₆₅ is, independently, an amino acid selected from the group consisting of A, C, G, S, Q, N, R, Y, E, K, D, H, M, V, I, and L;
each N₆₆ is, independently, an amino acid selected from the group consisting of V, I, A, T, S, G, R, P, Y, L, N, H, C, M, F, Q, E, K, and D;
each N₆₇ is, independently, an amino acid selected from the group consisting of S, N, Q, R, T, G, K, E, H, D, A, C, P, Y, M, V, W, I, F, and L;
each N₆₈ is, independently, an amino acid selected from the group consisting of K, R, H, S, G, N, Q, D, E, T, A, C, P, Y, M, V, W, I, L, and F;
each N₆₉ is, independently, an amino acid selected from the group consisting of K, R, H, S, G, N, Q, D, E, T, A, C, P, Y, M, V, W, I, L, and F;
each N₇₀ is, independently, an amino acid selected from the group consisting of D, E, Q, N, S, H, T, R, K, G, A, C, Y, P, M, V, W, I, F, and L;
each N₇₁ is, independently, an amino acid selected from the group consisting of A, T, C, G, S, Q, N, P, R, Y, E, K, D, H, M, V, W, I, L, and F.

20. The polypeptide of any one of claims 16-19, wherein m is 1 and Y₁ comprises an amino acid sequence having at least 90%, 91%, 92%, 93%, 94%, 95%, 96%, 97%, 98%, 99%, or 100% identity to an amino acid sequence selected from the group consisting of SEQ ID NO. 17, 18, 19, 20, 21, 22, 23, 24, 25, 27, 29, 34, 35, 36, 37, 38, 56, 57, 58, 74, or 75.

21. The polypeptide of any one of claims 16-20, wherein Z₁ is selected from the group consisting of an antiviral, insulin, an incretin, an enzyme, an enzyme inhibitor, a hormone, a cytokine, an antibody, an antimicrobial peptide, a mucosal protein, pesticide, bactericide herbicide, fungicide, nematicide, miticide, plant growth regulator, plant growth stimulator, or fertilizer), a vaccine, a diagnostic protein, a feed conversion enzyme, a flavoring, and a nutritional protein.

22. A yeast comprising a heterologous nucleic acid molecule encoding a polypeptide having a formula of (X₁)ₙ-(Y₁)ₘ-Z₁ wherein:
X₁ is the pre-protein signal peptide of any one of claims 1-11,
Y₁ is the pro-protein signal peptide of any one of claims 12-15, and
Z₁ is a payload protein,
wherein n is 0-1 and m is 0-1,
provided that n and m are not both 0.

23. The yeast of claim 22, wherein the yeast is selected from the group consisting of Kluyveromyces, Pichia, Saccharomyces, Trichoderma, and Aspergillus.

24. The yeast of claim 22, wherein the yeast is a Kluyveromyces yeast and X₁ comprises an amino acid sequence selected from Formula I or SEQ ID NO. 1 and Y₁ comprising an amino acid sequence selected from Formula VI, SEQ ID NO. 20 or SEQ ID NO. 21.

25. The yeast of claim 22, wherein the yeast is a Pichia yeast (e.g., P. pastoris) and X₁ comprises an amino acid sequence selected from Formula II or SEQ ID NO. 2, 3, 4, 5, 6, or 7 and Y₁ comprises an amino acid sequence selected from Formula VI, SEQ ID NO. 20 or SEQ ID NO. 21.

26. The yeast of claim 22, wherein the yeast is a Saccharomyces yeast and X₁ comprises an amino acid sequence selected from Formula III, Formula IV, or Formula V, or SEQ ID NO. 8, 9, 10, 11, 12, 13, 14, 15, or 16 and Y₁ comprises an amino acid sequence selected from Formula VI, Formula VII, or Formula VIII or SEQ ID NO. 18, 19, 20, 21, 22, 23, 24, or 25.

27. The yeast of claim 22, wherein the yeast is a Trichoderma yeast and X₁ comprises an amino acid sequence selected from Formula IX or SEQ ID NO. 31, 32, or 33 and Y₁ comprises an amino acid sequence selected from Formula X or Formula XI or SEQ ID NO. 34, 35, 36, 37, or 38.

28. The yeast of claim 22, wherein the yeast is an Aspergillus yeast (e.g., A. niger) and X₁ comprises an amino acid sequence selected from Formula XIII, or SEQ ID NO. 70, 71, 72, or 73 and Y₁ comprises an amino acid sequence selected from Formula XIV or Formula XV or SEQ ID NO. 74 or 75.

29. The yeast of any one of claims 22-28, wherein Z₁ is selected from the group consisting of an antiviral, insulin, an incretin, an enzyme, an enzyme inhibitor, a hormone, pesticide, a cytokine, an antibody, an antimicrobial peptide, a mucosal protein, bactericide herbicide, fungicide, nematicide, miticide, plant growth regulator, plant growth stimulator, or fertilizer), a vaccine, a diagnostic protein, a feed conversion enzyme, a flavoring, and a nutritional protein.

30. A method for producing a payload protein, comprising
i) transfecting a yeast with a nucleic acid encoding the polypeptide of any one of claims 16-21, producing an engineered yeast; and
ii) culturing the engineered yeast in an environment effective to grow the engineered yeast, and
iii) inducing secretion of the payload protein by the engineered yeast.

31. The method of claim 30, wherein inducing secretion of the payload protein comprises culturing the yeast under conditions sufficient to express the polypeptide of any one of claims 16-21, wherein the presence of the signal peptide induces secretion of the payload protein.

32. The method of claim 30 or 31, wherein the yeast is selected from the group consisting of Kluyveromyces, Pichia, Saccharomyces, Trichoderma, and Aspergillus.

33. The method of claim 30 or 31, wherein the yeast is a Kluyveromyces yeast and X₁ comprises an amino acid sequence selected from Formula I or SEQ ID NO. 1 and Y₁ comprises an amino acid sequence selected from Formula VI or SEQ ID NO. 20 or SEQ ID NO. 21.

34. The method of claim 30 or 31, wherein the yeast is a Pichia yeast (e.g., P. pastoris) and X₁ comprises an amino acid sequence selected from Formula II or SEQ ID NO. 2, 3, 4, 5, 6, or 7 and Y₁ comprises an amino acid sequence selected from Formula VI or SEQ ID NO. 20 or SEQ ID NO. 21.

35. The method of claim 30 or 31, wherein the yeast is a Saccharomyces yeast and X₁ comprises an amino acid sequence selected from Formula III, Formula IV, or Formula V, or SEQ ID NO. 8, 9, 10, 11, 12, 13, 14, 15, or 16 and Y₁ comprises an amino acid sequence selected from Formula VI, Formula VII, or Formula VIII, or SEQ ID NO. 18, 19, 20, 21, 22, 23, 24, or 25.

36. The method of claim 30 or 31, wherein the yeast is a Trichoderma yeast and X₁ comprises an amino acid sequence selected from Formula IX or SEQ ID NO. 31, 32, or 33 and Y₁ comprises an amino acid sequence selected from Formula X or Formula XI, or SEQ ID NO. 34, 35, 36, 37, or 38.

37. The method of claim 30 or 31, wherein the yeast is an Aspergillus yeast (e.g., A. niger) and X₁ comprises an amino acid sequence selected from Formula XIII or SEQ ID NO. 70, 71, 72, or 73 and Y₁ comprises an amino acid sequence selected from Formula XIV or Formula XV or SEQ ID NO. 74 or 75.

38. The method of any one of claims 29-37, wherein the yeast is grown in culture media and the method further comprises recovering the payload protein from the culture media.

39. The method of any of claims 29-38, wherein Z₁ is selected from the group consisting of an antiviral, insulin, an incretin, a cytokine, an antibody, an antimicrobial peptide, a mucosal protein, an enzyme, an enzyme inhibitor, a hormone, pesticide, bactericide herbicide, fungicide, nematicide, miticide, plant growth regulator, plant growth stimulator, fertilizer, a vaccine, a diagnostic protein, a feed conversion enzyme, a flavoring, or a nutritional protein.

40. A method for treating a disease or a condition in a subject in need thereof comprising administering to the subject a therapeutically effective amount of the yeast of any one of claims 22-29.

41. The method of claim 40, wherein the disease or condition is selected from an infection, an autoimmune disease, primary (congenital) enzymatic deficiency, enzymatic deficiencies secondary to functional gut disorders, diabetes, obesity, a metabolic disorder, intestinal bacterial overgrowth, enteric infection, bacterial vaginosis, inflammatory bowel disease, irritable bowel syndrome, small bowel syndrome, Celiac disease, gluten intolerance, colitis, peptic ulcer, or another GI condition or disorder.

42. The method of claim 40 or 41, wherein the disease or condition is an enzyme deficiency and the payload protein is an enzyme.

43. The method of claim 40 or 41, wherein the disease or condition is congenital sucrase-isomaltase deficiency and the payload protein is one or both of invertase and isomaltase.

44. The method of claim 40 or 41, wherein the disease or condition is one or both of sucrose and isomaltase intolerance secondary to a functional gut disorder and the payload protein is one or both of invertase and isomaltase.

45. The method of claim 40 or 41, wherein the disease or condition is one or more of gluten intolerance, refractory sprue, or Celiac disease and the payload protein is one or more of An-PEP, Mx-PEP, Aspergillus tubigensis prolyl endopeptidase, subtilisin, sedolisin, and larozotide.

46. The method of claim 40 or 41, wherein the disease or condition is pancreatitis or exocrine pancreatic insufficiency and the payload protein is selected from one or more of triacylglycerol lipase, colipase, alpha-amylase, trypsin, and chymotrypsin.

47. The method of claim 40 or 41, wherein the disease or condition is enteropeptidase deficiency or enterokinase deficiency and the payload protein is one or all of enteropeptidase, proenteropeptidase, and enterokinase.

48. The method of claim 40 or 41, wherein the disease or condition is small intestinal bacterial overgrowth, inflammatory bowel disease, irritable bowel syndrome, C. difficile infection, cystic fibrosis, necrotizing enterocolitis, and diabetes, and the payload protein is intestinal alkaline phosphatase.

49. The method of claim 40 or 41, wherein the disease or condition is short bowel syndrome and the payload protein is IGF-1, GLP-2, or a synthetic derivative of GLP-2.

50. The method of claim 40 or 41, wherein the disease or condition is lactose sensitivity or lactose intolerance and the payload protein is lactase.

51. The method of claim 40 or 41, wherein the disease or condition is trehalose sensitivity or lactose intolerance and the payload protein is trehalase.

52. The method of claim 40 or 41, wherein the disease or condition is maltose sensitivity or lactose intolerance and the payload protein is maltase.

53. The method of claim 40 or 41, wherein the disease or condition is pernicious anemia and the payload protein is intrinsic factor.

54. The method of claim 40 or 41, wherein the disease or condition is bacterial overgrowth and the payload protein is lysozyme, nisin, a defensin, magainin, cateslytin, or any combination thereof.

55. The method of claim 40 or 41, wherein the condition is a bacterial infection caused by one or more of E. coli, C. difficile, Vibrio cholera, Shigella, Salmonella, Cryptosporidium, or any combination thereof.

56. The method of claim 40 or 41, wherein the condition is a viral infection.

57. The method of claim 40 or 41, wherein the disease or condition is type 1 or type 2 diabetes mellitus and the payload protein is insulin, or an incretin.

58. The method of claim 40 or 41, wherein the administering is oral or topical.

59. The method of claim 40 or 41, wherein the disease or condition has an inflammatory component and the payload protein is IL-10, IL-22, TGFβ, an anti-TNFα antibody or fragment thereof, or any combination thereof.


SEQUENCE LISTING (106 sequences)

SEQ ID NO: 1 - protein, 21 aa, Artificial Sequence
  Description of Artificial Sequence Synthetic peptide
MKLSSLLLLLLLLLSSLVLAA

SEQ ID NO: 2 - protein, 19 aa, Artificial Sequence
  Description of Artificial Sequence Synthetic peptide
MRFPSIFTAVLFAASSALA

SEQ ID NO: 3 - protein, 23 aa, Artificial Sequence
  Description of Artificial Sequence Synthetic peptide
MFSPILSLEIILALATLQSVFAR

SEQ ID NO: 4 - protein, 22 aa, Artificial Sequence
  Description of Artificial Sequence Synthetic peptide
  MOD_RES (21)..(21): A or absent
  MOD_RES (22)..(22): S or absent
  SITE (21)..(22): This region may encompass 0-2 residues
  See specification as filed for detailed description of substitutions and preferred embodiments
MKLSTLLLTLLLLLLALVLAXX

SEQ ID NO: 5 - protein, 22 aa, Artificial Sequence
  Description of Artificial Sequence Synthetic peptide
MLKLLLLILLLLLLVSLVLAAS

SEQ ID NO: 6 - protein, 24 aa, Artificial Sequence
  Description of Artificial Sequence Synthetic peptide
  MOD_RES (23)..(23): A or absent
  MOD_RES (24)..(24): S or absent
  SITE (23)..(24): This region may encompass 0-2 residues
  See specification as filed for detailed description of substitutions and preferred embodiments
MKLLLLLLLLLLLLLLLALVLAXX

SEQ ID NO: 7 - protein, 15 aa, Artificial Sequence
  Description of Artificial Sequence Synthetic peptide
MKLLLLLLSLVLAAS

SEQ ID NO: 8 - protein, 15 aa, Artificial Sequence
  Description of Artificial Sequence Synthetic peptide
MKLSSLLLALLLALA

SEQ ID NO: 9 - protein, 22 aa, Artificial Sequence
  Description of Artificial Sequence Synthetic peptide
  MOD_RES (21)..(21): A or absent
  MOD_RES (22)..(22): P or absent
  SITE (21)..(22): This region may encompass 0-2 residues
  See specification as filed for detailed description of substitutions and preferred embodiments
MKLSSLLLALLLALASLALAXX

SEQ ID NO: 10 - protein, 23 aa, Artificial Sequence
  Description of Artificial Sequence Synthetic peptide
  MOD_RES (23)..(23): K or absent
  See specification as filed for detailed description of substitutions and preferred embodiments
MKLSSLLLALLLALASLALAAPX

SEQ ID NO: 11 - protein, 21 aa, Artificial Sequence
  Description of Artificial Sequence Synthetic peptide
MKLKTVRSAVLSSLFASQVLG

SEQ ID NO: 12 - protein, 23 aa, Artificial Sequence
  Description of Artificial Sequence Synthetic peptide
MKFLSLLLALVAALALALALAAP

SEQ ID NO: 13 - protein, 19 aa, Artificial Sequence
  Description of Artificial Sequence Synthetic peptide
MLLQAFLFLLAGFAAKISA

SEQ ID NO: 14 - protein, 22 aa, Artificial Sequence
  Description of Artificial Sequence Synthetic peptide
MKFKLTLLAALLALAALVLAAS

SEQ ID NO: 15 - protein, 20 aa, Artificial Sequence
  Description of Artificial Sequence Synthetic peptide
MKLSSILLLLALLALVLAAS

SEQ ID NO: 16 - protein, 21 aa, Artificial Sequence
  Description of Artificial Sequence Synthetic peptide
MKLLSLLALLLLLASLVLAAS

SEQ ID NO: 17 - protein, 67 aa, Artificial Sequence
  Description of Artificial Sequence Synthetic polypeptide
APVNTTTEDETAQIPAEAVIGYSDLEGDFDVAVLPFSNSTNNGLLFINTTIASIAAKEEGVSLEKRE

SEQ ID NO: 18 - protein, 77 aa, Artificial Sequence
  Description of Artificial Sequence Synthetic polypeptide
  MOD_RES (69)..(69): D or absent
  MOD_RES (70)..(70): K or absent
  MOD_RES (71)..(71): R or absent
  MOD_RES (72)..(72): E or absent
  MOD_RES (73)..(73): E or absent
  MOD_RES (74)..(74): G or absent
  MOD_RES (75)..(75): E or absent
  MOD_RES (76)..(76): P or absent
  MOD_RES (77)..(77): K or absent
  SITE (69)..(77): This region may encompass 0-9 residues
  See specification as filed for detailed description of substitutions and preferred embodiments
SLALAAPVNTTTEDETAQIPAEAVIGYSDLEGDFDVAVLPFSNSTNNGLLFINTTIASIAAKEEGVSLXXXXXXXXX

SEQ ID NO: 19 - protein, 50 aa, Artificial Sequence
  Description of Artificial Sequence Synthetic polypeptide
  MOD_RES (43)..(43): K or absent
  MOD_RES (44)..(44): R or absent
  MOD_RES (45)..(45): E or absent
  MOD_RES (46)..(46): E or absent
  MOD_RES (47)..(47): G or absent
  MOD_RES (48)..(48): E or absent
  MOD_RES (49)..(49): P or absent
  MOD_RES (50)..(50): K or absent
  SITE (43)..(44): This region may encompass 0-2 residues
  SITE (45)..(50): This region may encompass 0-6 residues
  See specification as filed for detailed description of substitutions and preferred embodiments
QPIDDTESQTTSVNLMADDTESAFATQTNSGGLDVVGLISMAXXXXXXXX

SEQ ID NO: 20 - protein, 27 aa, Artificial Sequence
  Description of Artificial Sequence Synthetic peptide
  MOD_RES (20)..(20): K or absent
  MOD_RES (21)..(21): R or absent
  MOD_RES (22)..(22): E or absent
  MOD_RES (23)..(23): E or absent
  MOD_RES (24)..(24): G or absent
  MOD_RES (25)..(25): E or absent
  MOD_RES (26)..(26): P or absent
  MOD_RES (27)..(27): K or absent
  SITE (20)..(27): This region may encompass 0-8 residues
  See specification as filed for detailed description of substitutions and preferred embodiments
IPLVANVSFNSDNGSQWLYXXXXXXXX

SEQ ID NO: 21 - protein, 27 aa, Artificial Sequence
  Description of Artificial Sequence Synthetic peptide
  MOD_RES (20)..(20): K or absent
  MOD_RES (21)..(21): R or absent
  MOD_RES (22)..(22): D or absent
  MOD_RES (23)..(23): V or absent
  MOD_RES (24)..(24): V or absent
  MOD_RES (25)..(25): Y or absent
  MOD_RES (26)..(26): P or absent
  MOD_RES (27)..(27): K or absent
  SITE (20)..(27): This region may encompass 0-8 residues
  See specification as filed for detailed description of substitutions and preferred embodiments
IPLVANVSFNSDNGSQWLYXXXXXXXX

SEQ ID NO: 22 - protein, 42 aa, Artificial Sequence
  Description of Artificial Sequence Synthetic polypeptide
EPWSTLTVTRSTYDEITDTDYNSTGIAVNPYTVSASRHKRDV

SEQ ID NO: 23 - protein, 28 aa, Artificial Sequence
  Description of Artificial Sequence Synthetic peptide
STLTPSVVFIGGGLTEETTFGIRHKRDV

SEQ ID NO: 24 - protein, 42 aa, Artificial Sequence
  Description of Artificial Sequence Synthetic polypeptide
DPWSTTTSIYSLGGTTSYVSEFGLSISDETVTEMKSRHKRDV

SEQ ID NO: 25 - protein, 72 aa, Artificial Sequence
  Description of Artificial Sequence Synthetic polypeptide
  MOD_RES (64)..(64): D or absent
  MOD_RES (65)..(65): K or absent
  MOD_RES (66)..(66): R or absent
  MOD_RES (67)..(67): E or absent
  MOD_RES (68)..(68): E or absent
  MOD_RES (69)..(69): G or absent
  MOD_RES (70)..(70): E or absent
  MOD_RES (71)..(71): P or absent
  MOD_RES (72)..(72): K or absent
  SITE (64)..(72): This region may encompass 0-9 residues
  See specification as filed for detailed description of substitutions and preferred embodiments
APVNTTTEDETAQIPAEAVIGYSDLEGDFDVAVLPFSNSTNNGLLFINTTIASIAAKEEGVSLXXXXXXXXX

SEQ ID NO: 26 - protein, 25 aa, Artificial Sequence
  Description of Artificial Sequence Synthetic peptide
FCTFFEKHHRKWDILLEKSTGVMEA

SEQ ID NO: 27 - protein, 68 aa, Artificial Sequence
  Description of Artificial Sequence Synthetic polypeptide
  MOD_RES (64)..(64): D or absent
  MOD_RES (65)..(65): K or E or absent
  MOD_RES (66)..(66): R or K or absent
  MOD_RES (67)..(67): R or absent
  MOD_RES (68)..(68): E or absent
  SITE (64)..(68): This region may encompass 0-5 residues
  See specification as filed for detailed description of substitutions and preferred embodiments
APVNTTTEDETAQIPAEAVIGYSDLEGDFDVAVLPFSNSTNNGLLFINTTIASIAAKEEGVSLXXXXX

SEQ ID NO: 28 - protein, 19 aa, Artificial Sequence
  Description of Artificial Sequence Synthetic peptide
MKFSTILAASTALISVVMA

SEQ ID NO: 29 - protein, 64 aa, Artificial Sequence
  Description of Artificial Sequence Synthetic polypeptide
APVSTETDIDDLPISVPEEALIGFIDLTGDEVSLLPVNNGTHTGILFLNTTIAEAAFADKDDLE

SEQ ID NO: 30 - protein, 23 aa, Artificial Sequence
  Description of Artificial Sequence Synthetic peptide
MFSPILSLEIILALATLQSVFAR

SEQ ID NO: 31 - protein, 21 aa, Artificial Sequence
  Description of Artificial Sequence Synthetic peptide
  MOD_RES (20)..(20): A or absent
  MOD_RES (21)..(21): P or absent
  SITE (20)..(21): This region may encompass 0-2 residues
  See specification as filed for detailed description of substitutions and preferred embodiments
MKSSLLLLALLALAALASAXX

SEQ ID NO: 32 - protein, 20 aa, Artificial Sequence
  Description of Artificial Sequence Synthetic peptide
  MOD_RES (19)..(19): A or absent
  MOD_RES (20)..(20): P or absent
  SITE (19)..(20): This region may encompass 0-2 residues
  See specification as filed for detailed description of substitutions and preferred embodiments
MKSSLLLLLLALASLALAXX

SEQ ID NO: 33 - protein, 23 aa, Artificial Sequence
  Description of Artificial Sequence Synthetic peptide
  MOD_RES (22)..(22): A or absent
  MOD_RES (23)..(23): P or absent
  SITE (22)..(23): This region may encompass 0-2 residues
  See specification as filed for detailed description of substitutions and preferred embodiments
MKSSSLLLLALLALLAALASAXX

SEQ ID NO: 34 - protein, 48 aa, Artificial Sequence
  Description of Artificial Sequence Synthetic polypeptide
EARDSWASPGGQSTRADDAINVAGTGGSILRPAKFSTLDSRRSKRSSE

SEQ ID NO: 35 - protein, 26 aa, Artificial Sequence
  Description of Artificial Sequence Synthetic peptide
PSVRPPVASRDYSHSARWSTKKRSRR

SEQ ID NO: 36 - protein, 25 aa, Artificial Sequence
  Description of Artificial Sequence Synthetic peptide
LHVFSTDYLLIYIVGDLPRRVKRSV

SEQ ID NO: 37 - protein, 120 aa, Artificial Sequence
  Description of Artificial Sequence Synthetic polypeptide
SLSPVDNILLLQLLGTAAAVLIGASKNDGGVTRVEDRSYSEILFSLNPLSDTRLRLKHVVAVAGGKASSVTDYYLSLSRNVPEGVWYMATSIDGEIAWLGLIADVLRGVFLLGEAARESV

SEQ ID NO: 38 - protein, 96 aa, Artificial Sequence
  Description of Artificial Sequence Synthetic polypeptide
SYSRVSNAAGKSRGDCRSRLFPDGTKSPPSASPRREARRDSLHPVLTSSAESGSGSYMSSNSARDLLRSGAKNHLGAPNNDQVKFGLATRTRKRAE

SEQ ID NO: 39 - DNA, 59 bp, Artificial Sequence
  Description of Artificial Sequence Synthetic oligonucleotide
atgaaattgtcttctttgttgttgttgttgttgttgttgttgtcttctttggttttgct

SEQ ID NO: 40 - DNA, 69 bp, Artificial Sequence
  Description of Artificial Sequence Synthetic oligonucleotide
atgttctctccaattttgtccttggaaattattttagctttggctactttgcaatctgtcttcgctcga

SEQ ID NO: 41 - DNA, 66 bp, Artificial Sequence
  Description of Artificial Sequence Synthetic oligonucleotide
atgaaattgtctactctgttgttgactttgttgttgttgttgttggctttggttttggctgcttct

SEQ ID NO: 42 - DNA, 66 bp, Artificial Sequence
  Description of Artificial Sequence Synthetic oligonucleotide
atgttgaaattgttgctgttgattttgttgttgttgcttttggtttctttggttttggctgcttct

SEQ ID NO: 43 - DNA, 72 bp, Artificial Sequence
  Description of Artificial Sequence Synthetic oligonucleotide
atgaaattgttactgcttttacttcttttgctgttattgttgcttttgctggctttggttttggctgcttct

SEQ ID NO: 44 - DNA, 45 bp, Artificial Sequence
  Description of Artificial Sequence Synthetic oligonucleotide
atgaaattgttgttgttgttgttgtctttggttttggctgcttct

SEQ ID NO: 45 - DNA, 69 bp, Artificial Sequence; identical to SEQ ID NO: 40
  Description of Artificial Sequence Synthetic oligonucleotide

SEQ ID NO: 46 - DNA, 45 bp, Artificial Sequence
  Description of Artificial Sequence Synthetic oligonucleotide
atgaagttatcttctttattgctggctctgcttctagccttggcg

SEQ ID NO: 47 - DNA, 60 bp, Artificial Sequence
  Description of Artificial Sequence Synthetic oligonucleotide
atgaagttatcttctttattgctggctctgcttctagccttggcgtctctagcgctggcc

SEQ ID NO: 48 - DNA, 69 bp, Artificial Sequence
  Description of Artificial Sequence Synthetic oligonucleotide
atgaagttatcttctttattgctggctctgcttctagccttggcgtctctagcgctggccgcaccaaag

SEQ ID NO: 49 - DNA, 63 bp, Artificial Sequence
  Description of Artificial Sequence Synthetic oligonucleotide
atgaaattgaaaactgttagatctgctgttttgtcttctttgtttgcttctcaagttttgggt

SEQ ID NO: 50 - DNA, 201 bp, Artificial Sequence
  Description of Artificial Sequence Synthetic polynucleotide
gctccagtcaacactacaacagaagatgaaacggcacaaattccggctgaagctgtcatcggttactcagatttagaaggggatttcgatgttgctgttttgccattttccaacagcacaaataacgggttattgtttataaatactactattgccagcattgctgctaaagaagaaggggtatctctcgagaaaagagag

SEQ ID NO: 51 - DNA, 150 bp, Artificial Sequence
  Description of Artificial Sequence Synthetic polynucleotide
gctccagttaatactactactgaagatgaaactgctcaaattccagctgaagctgttattggttattctgatttggagggtgactttgatgttgctgttttgccattttctaactctactaacaacggtttgctattcatcaacactact

SEQ ID NO: 52 - DNA, 150 bp, Artificial Sequence
  Description of Artificial Sequence Synthetic polynucleotide
caaccaattgatgatactgaatctcaaactacttctgttaatttgatggctgatgatactgaatctgcttttgctactcaaactaattctggtggtttggatgttgttggtttgatttctatggctaaaagagaagaaggtgaaccaaaa

SEQ ID NO: 53 - DNA, 87 bp, Artificial Sequence
  Description of Artificial Sequence Synthetic oligonucleotide
gctccaattccattggttgctaatgtttcttttaattctgataatggttctcaatggttgtataaaagagaagaaggtgaaccaaaa

SEQ ID NO: 54 - DNA, 87 bp, Artificial Sequence
  Description of Artificial Sequence Synthetic oligonucleotide
gctccaattccattggttgctaatgtttcttttaattctgataatggttctcaatggttgtataaaagagatgttgtttatccaaaa

SEQ ID NO: 55 - protein, 18 aa, Artificial Sequence
  Description of Artificial Sequence Synthetic peptide
MRSLLILVLCFLPLAALG

SEQ ID NO: 56 - protein, 66 aa, Artificial Sequence
  Description of Artificial Sequence Synthetic polypeptide
APVSTETDIDDLPISVPEEALIGFIDLTGDEVSLLPVNNGTHTGILFLNTTIAEAAFADKDDLEKR

SEQ ID NO: 57 - protein, 29 aa, Artificial Sequence
  Description of Artificial Sequence Synthetic peptide
APIPLVANVSFNSDNGSQWLYKRDVVYPK

SEQ ID NO: 58 - protein, 29 aa, Artificial Sequence
  Description of Artificial Sequence Synthetic peptide
APIPLVANVSFNSDNGSQWLYKREEGEPK

SEQ ID NO: 59 - protein, 585 aa, Artificial Sequence
  Description of Artificial Sequence Synthetic polypeptide
APVNTTTEDETAQIPAEAVIGYSDLEGDFDVAVLPFSNSTNNGLLFINTTIASIAAKEEGVSLDKREEGEPKSMTNETSDRPLVHFTPNKGWMNDPNGLWYDEKDAKWHLYFQYNPNDTVWGTPLFWGHATSDDLTNWEDQPIAIAPKRNDSGAFSGSMVVDYNNTSGFFNDTIDPRQRCVAIWTYNTPESEEQYISYSLDGGYTFTEYQKNPVLAANSTQFRDPKVFWYEPSQKWIMTAAKSQDYKIEIYSSDDLKSWKLESAFANEGFLGYQYECPGLIEVPTEQDPSKSYWVMFISINPGAPAGGSFNQYFVGSFNGTHFEAFDNQSRVVDFGKDYYALQTFFNTDPTYGSALGIAWASNWEYSAFVPTNPWRSSMSLVRKFSLNTEYQANPETELINLKAEPILNISNAGPWSRFATNTTLTKANSYNVDLSNSTGTLEFELVYAVNTTQTISKSVFADLSLWFKGLEDPEEYLRMGFEVSASSFFLDRGNSKVKFVKENPYFTNRMSVNNQPFKSENDLSYYKVYGLLDQNILELYFNDGDVVSTNTYFMTTGNALGSVNMTTGVDNLFYIDKFQVREVK

SEQ ID NO: 60 - protein, 513 aa, Artificial Sequence
  Description of Artificial Sequence Synthetic polypeptide
SMTNETSDRPLVHFTPNKGWMNDPNGLWYDEKDAKWHLYFQYNPNDTVWGTPLFWGHATSDDLTNWEDQPIAIAPKRNDSGAFSGSMVVDYNNTSGFFNDTIDPRQRCVAIWTYNTPESEEQYISYSLDGGYTFTEYQKNPVLAANSTQFRDPKVFWYEPSQKWIMTAAKSQDYKIEIYSSDDLKSWKLESAFANEGFLGYQYECPGLIEVPTEQDPSKSYWVMFISINPGAPAGGSFNQYFVGSFNGTHFEAFDNQSRVVDFGKDYYALQTFFNTDPTYGSALGIAWASNWEYSAFVPTNPWRSSMSLVRKFSLNTEYQANPETELINLKAEPILNISNAGPWSRFATNTTLTKANSYNVDLSNSTGTLEFELVYAVNTTQTISKSVFADLSLWFKGLEDPEEYLRMGFEVSASSFFLDRGNSKVKFVKENPYFTNRMSVNNQPFKSENDLSYYKVYGLLDQNILELYFNDGDVVSTNTYFMTTGNALGSVNMTTGVDNLFYIDKFQVREVK

SEQ ID NO: 61 - protein, 130 aa, Artificial Sequence
  Description of Artificial Sequence Synthetic polypeptide
KVFERCELARTLKRLGMDGYRGISLANWMCLAKWESGYNTRATNYNAGDRSTDYGIFQINSRYWCNDGKTPGAVNACQLSCSALLQDNIADAVACAKRVVRDPQGIRAWVAWRNRCQNRDVRQYVQGCGV

SEQ ID NO: 62 - protein, 985 aa, Artificial Sequence
  Description of Artificial Sequence Synthetic polypeptide
IKHRLNGFTILEHPDPAKRDLLQDIVTWDDKSLFINGERIMLFSGEVHPFRLPVPSLWLDIFHKIRALGFNCVSFYIDWALLEGKPGDYRAEGIFALEPFFDAAKEAGIYLIARPGSYINAEVSGGGFPGWLQRVNGTLRSSDEPFLKATDNYIANAAAAVAKAQITNGGPVILYQPENEYSGGCCGVKYPDADYMQYVMDQARKADIVVPFISNDASPSGHNAPGSGTSAVDIYGHDSYPLGFDCANPSVWPEGKLPDNFRTLHLEQSPSTPYSLLEFQAGAFDPWGGPGFEKCYALVNHEFSRVFYRNDLSFGVSTFNLYMTFGGTNWGNLGHPGGYTSYDYGSPITETRNVTREKYSDIKLLANFVKASPSYLTATPRNLTTGVYTDTSDLAVTPLIGDSPGSFFVVRHTDYSSQESTSYKLKLPTSAGNLTIPQLEGTLSLNGRDSKIHVVDYNVSGTNIIYSTAEVFTWKKFDGNKVLVLYGGPKEHHELAIASKSNVTIIEGSDSGIVSTRKGSSVIIGWDVSSTRRIVQVGDLRVFLLDRNSAYNYWVPELPTEGTSPGFSTSKTTASSIIVKAGYLLRGAHLDGADLHLTADFNATTPIEVIGAPTGAKNLFVNGEKASHTVDKNGIWSSEVKYAAPEIKLPGLKDLDWKYLDTLPEIKSSYDDSAWVSADLPKTKNTHRPLDTPTSLYSSDYGFHTGYLIYRGHFVANGKESEFFIRTQGGSAFGSSVWLNETYLGSWTGADYAMDGNSTYKLSQLESGKNYVITVVIDNLGLDENWTVGEETMKNPRGILSYKLSGQDASAITWKLTGNLGGEDYQDKVRGPLNEGGLYAERQGFHQPQPPSESWESGSPLEGLSKPGIGFYTAQFDLDLPKGWDVPLYFNFGNNTQAARAQLYVNGYQYGKFTGNVGPQTSFPVPEGILNYRGTNYVALSLWALESDGAKLGSFELSYTTPVLTGYGNVESPEQPKYEQRKGAY

SEQ ID NO: 63 - protein, 114 aa, Artificial Sequence
  Description of Artificial Sequence Synthetic polypeptide
EVQLVESGGGLVQPGGSLRLSCAASGFTFSDYWMYWVRQAPGKGLEWVSEINTNGLITKYPDSVGRFTISRDNAKNTLYLQMNSLRPEDTAVYYCARSPSGFNRGQGTLVTVSS

SEQ ID NO: 64 - protein, 369 aa, Artificial Sequence
  Description of Artificial Sequence Synthetic polypeptide
IEEGKLVIWINGDKGYNGLAEVGKKFEKDTGIKVTVEHPDKLEEKFPQVAATGDGPDIIFWAHDRFGGYAQSGLLAEITPDKAFQDKLYPFTWDAVRYNGKLIAYPIAVEALSLIYNKDLLPNPPKTWEEIPALDKELKAKGKSALMFNLQEPYFTWPLIAADGGYAFKYENGKYDIKDVGVDNAGAKAGLTFLVDLIKNKHMNADTDYSIAEAAFNKGETAMTINGPWAWSNIDTSKVNYGVTVLPTFKGQPSKPFVGVLSAGINAASPNKELAKEFLENYLLTDEGLEAVNKDKPLGAVALKSYEEELAKDPRIAATMENAQKGEIMPNIPQMSAFWYAVRTAVINAASGRQTVDEALKDAQTRITK

SEQ ID NO: 65 - protein, 411 aa, Artificial Sequence
  Description of Artificial Sequence Synthetic polypeptide
AQSEPELKLESVVIVSRHGVRAPTKATQLMQDVTPDAWPTWPVKLGELTPRGGELLAYLGHYWRQRLVADGLLPKCGCPQSGQVAILADVDERTRKTGEAFAAGLAPDCAITVHTQADTSSPDPLFNPLKTGVCQLDNANVTDAILERAGGSLADFTGHYQTAFRELERVLNFPQSNLCLKREKQDESCSLTQALPSELKVSADCVSLTGAVSLASMLTEIFLLQQAQGMPEPGWGRITDSHQWNTLLSLHNAQFDLLQRTPEVARSRATPLLDLIKTALTPHPPQKQAYGVTLPTSVLFLAGHDTNLANLGGALELNWTLPGQPDNTPPGGELVFERWRRLSDNSQWIQVSLVFQTLQQMRDKTPLSLNTPPGEVKLTLAGCEERNAQGMCSLAGFTQIVNEARIPACSL

SEQ ID NO: 66 - protein, 53 aa, Artificial Sequence
  Description of Artificial Sequence Synthetic polypeptide
FVNQHLCGSHLVEALYLVCGERGFFYTPKEWKGIVEQCCTSICSLYQLENYCN

SEQ ID NO: 67 - protein, 105 aa, Artificial Sequence
  Description of Artificial Sequence Synthetic polypeptide
GPETLCGAELVDALQFVCGPRGFYFNKPTGYGSSIRRAPQTGIVDECCFRSCDLRRLEMYCAPLKPTKAARSIRAQRHTDMPKTQKEVHLKNTSRGSAGNKTYRM

SEQ ID NO: 68 - protein, 6 aa, Artificial Sequence
  Description of Artificial Sequence Synthetic peptide
NVISKR

SEQ ID NO: 69 - protein, 6 aa, Artificial Sequence
  Description of Artificial Sequence Synthetic peptide
SDVTKR

SEQ ID NO: 70 - protein, 23 aa, Artificial Sequence
  Description of Artificial Sequence Synthetic peptide
MRSLSLALLLLLALLASLALAAP

SEQ ID NO: 71 - protein, 23 aa, Artificial Sequence
  Description of Artificial Sequence Synthetic peptide
MRLSLSLLLLLLALLASLALAAP

SEQ ID NO: 72 - protein, 23 aa, Artificial Sequence
  Description of Artificial Sequence Synthetic peptide
MRLSSLLLGLLLALAASLALAAP

SEQ ID NO: 73 - protein, 23 aa, Artificial Sequence
  Description of Artificial Sequence Synthetic peptide
MRLSLLLALLALLALASLALAAP

SEQ ID NO: 74 - protein, 67 aa, Artificial Sequence
  Description of Artificial Sequence Synthetic polypeptide
ASSGRSPTITGQVSTLSSTDGTLPTSFTSGSAAGTISSTLPSNVTSTLGTIDLSPNGSADSSSKRST

SEQ ID NO: 75 - protein, 73 aa, Artificial Sequence
  Description of Artificial Sequence Synthetic polypeptide
SPTTSPSTTASLVSTSVTSSVTLTSTDVTTSEDTTGFVLPDSGTSSGTADALEAYSIGITSSSAVVDSKKRDA

SEQ ID NO: 76 - protein, 18 aa, Artificial Sequence
  Description of Artificial Sequence Synthetic peptide
MSFRSLLALSGLVCTGLA

SEQ ID NO: 77 - protein, 640 aa, Artificial Sequence
  Description of Artificial Sequence Synthetic polypeptide
MSFRSLLALSGLVCTGLANVISKRATLDSWLSNEATVARTAILNNIGADGAWVSGADSGIVVASPSTDNPDYFYTWTRDSGLVLKTLVDLFRNGDTSLLSTIENYISAQAIVQGISNPSGDLSSGAGLGEPKFNVDETAYTGSWGRPQRDGPALRATAMIGFGQWLLDNGYTSTATDIVWPLVRNDLSYVAQYWNQTGYDLWEEVNGSSFFTIAVQHRALVEGSAFATAVGSSCSWCDSQAPEILCYLQSFWTGSFILANFDSSRSGKDANTLLGSIHTFDPEAACDDSTFQPCSPRALANHKEVVDSFRSIYTLNDGLSDSEAVAVGRYPEDTYYNGNPWFLCTLAAAEQLYDALYQWDKQGSLEVTDVSLDFFKALYSDAATGTYSSSSSTYSSIVDAVKTFADGFVSIVETHAASNGSMSEQYDKSDGEQLSARDLTWSYAALLTANNRRNSVVPASWGETSASSVPGTCAATSAIGTYSSVTVTSWPSIVATGGTTTTATPTGSGSVTSTSKTTATASKTSTSTSSTSCTTPTAVAVTFDLTATTTYGENIYLVGSISQLGDWETSDGIALSADKYTSSDPLWYVTVTLPAGESFEYKFIRIESDDSVEWESDPNREYTVPQACGTSTATVTDTWR

SEQ ID NO: 78 - protein, 6 aa, Artificial Sequence
  Description of Artificial Sequence Synthetic peptide
EEGEPK

SEQ ID NO: 79 - protein, 6 aa, Artificial Sequence
  Description of Artificial Sequence Synthetic peptide
DVVYPK

SEQ ID NO: 80 - protein, 8 aa, Artificial Sequence
  Description of Artificial Sequence Synthetic peptide
DKREEGPK

SEQ ID NO: 81 - protein, 7 aa, Artificial Sequence
  Description of Artificial Sequence Synthetic peptide
KREEGPK

SEQ ID NO: 82 - protein, 7 aa, Artificial Sequence
  Description of Artificial Sequence Synthetic peptide
DKREKRE

SEQ ID NO: 83 - protein, 3 aa, Artificial Sequence
  Description of Artificial Sequence Synthetic peptide
DKR

SEQ ID NO: 84 - protein, 25 aa, Artificial Sequence
  Description of Artificial Sequence Synthetic peptide
  MOD_RES (6)..(6): Any amino acid except Cys
  MOD_RES (12)..(12): Any amino acid
  MOD_RES (18)..(18): Any amino acid except Cys
  See specification as filed for detailed description of substitutions and preferred embodiments
KKAAAXAAAAAXAAWAAXAAAKKKK

SEQ ID NO: 85 - protein, 130 aa, Artificial Sequence; identical to SEQ ID NO: 61
  Description of Artificial Sequence Synthetic polypeptide

SEQ ID NO: 86 - protein, 25 aa, Artificial Sequence
  Description of Artificial Sequence Synthetic peptide
IPLVANVSFNSDNGSQWLYKRDVVY

SEQ ID NO: 87 - protein, 11 aa, Artificial Sequence
  Description of Artificial Sequence Synthetic peptide
TLLLTLLLLLL

SEQ ID NO: 88 - protein, 11 aa, Artificial Sequence
  Description of Artificial Sequence Synthetic peptide
LLLILLLLLLV

SEQ ID NO: 89 - protein, 8 aa, Artificial Sequence
  Description of Artificial Sequence Synthetic peptide
LLALLLAL

SEQ ID NO: 90 - protein, 12 aa, Artificial Sequence
  Description of Artificial Sequence Synthetic peptide
LLALVAALALAL

SEQ ID NO: 91 - protein, 10 aa, Artificial Sequence
  Description of Artificial Sequence Synthetic peptide
LAALLALAAL

SEQ ID NO: 92 - protein, 8 aa, Artificial Sequence
  Description of Artificial Sequence Synthetic peptide
LLLALLAL

SEQ ID NO: 93 - protein, 6 aa, Artificial Sequence
  Description of Artificial Sequence Synthetic peptide
LLLASL

SEQ ID NO: 94 - protein, 12 aa, Artificial Sequence
  Description of Artificial Sequence Synthetic peptide
LLLLALLALAAL

SEQ ID NO: 95 - protein, 12 aa, Artificial Sequence
  Description of Artificial Sequence Synthetic peptide
SLLLLLLALASL

SEQ ID NO: 96 - protein, 14 aa, Artificial Sequence
  Description of Artificial Sequence Synthetic peptide
SLLLLALLALLAAL

SEQ ID NO: 97 - protein, 12 aa, Artificial Sequence
  Description of Artificial Sequence Synthetic peptide
ALLLLLALLASL

SEQ ID NO: 98 - protein, 12 aa, Artificial Sequence
  Description of Artificial Sequence Synthetic peptide
LLLLLLALLASL

SEQ ID NO: 99 - protein, 12 aa, Artificial Sequence
  Description of Artificial Sequence Synthetic peptide
LLGLLLALAASL

SEQ ID NO: 100 - protein, 14 aa, Artificial Sequence
  Description of Artificial Sequence Synthetic peptide
LLLALLALLALASL

SEQ ID NO: 101 - protein, 4 aa, Artificial Sequence
  Description of Artificial Sequence Synthetic peptide
LALA

SEQ ID NO: 102 - protein, 4 aa, Artificial Sequence
  Description of Artificial Sequence Synthetic peptide
LAVC

SEQ ID NO: 103 - protein, 6 aa, Artificial Sequence
  Description of Artificial Sequence Synthetic peptide
LALALA

SEQ ID NO: 104 - protein, 6 aa, Artificial Sequence
  Description of Artificial Sequence Synthetic peptide
LAVCHP

SEQ ID NO: 105 - protein, 6 aa, Artificial Sequence
  Description of Artificial Sequence Synthetic 6xHis tag
HHHHHH

SEQ ID NO: 106 - protein, 6 aa, Artificial Sequence
  Description of Artificial Sequence Synthetic peptide
KLLSLL